(19) United States
(12) Patent Application Publication
(10) Pub. No.: US 2008/0234262 A1
(43) Pub. Date: Sep. 25, 2008

(54) PYRAZOLOPYRIMIDINE ANALOGS AND THEIR USE AS MTOR KINASE AND PI3 KINASE INHIBITORS

(75) Inventors: Arie Zask, New York, NY (US); Pawel Wojciech Nowak, Montvale, NJ (US); Jeroen Cunera Verheijen, Highland Mills, NY (US); Kevin J. Curran, Congers, NY (US); Joshua Kaplan, Nyack, NY (US); David Malwitz, Hopewell Junction, NY (US); Matthew Gregory Bursavich, Sandy, UT (US); Derek Cecil Cole, New City, NY (US); Semiramis Ayral-Kaloustian, Tarrytown, NY (US); Ker Yu, Pine Brook, NJ (US); David James Richard, Ubrwick, NY (US); Mark Lefever, Tuscon, AZ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/050,445

(22) Filed: Mar. 18, 2008

Related U.S. Application Data
(60) Provisional application No. 60/919,301, filed on Mar. 21, 2007.

(51) Int. Cl. A61K 31/5377; A61K 31/519; A61P 35/00; C07D 413/14; C07D 487/04

(52) U.S. Cl. 514/234.2; 544/118; 544/262; 514/262.1

(57) ABSTRACT

The present invention relates to Pyrazolopyrimidine Analogs, methods of making Pyrazolopyrimidine Analogs, compositions comprising a Pyrazolopyrimidine Analog, and methods for treating mTOR-related disorders comprising administering to a subject in need thereof an effective amount of a Pyrazolopyrimidine Analog. The invention also relates to treating PI3K-related disorders comprising administering to a subject in need thereof an effective amount of a Pyrazolopyrimidine Analog.

### FIELD OF THE INVENTION

The invention relates to Pyrazolopyrimidine Analogs, compositions comprising a Pyrazolopyrimidine Analog, and methods for treating or preventing mTOR-related diseases comprising the administration of an effective amount of a Pyrazolopyrimidine Analog. The invention also relates to methods for treating or preventing PI3K-related diseases comprising the administration of an effective amount of a Pyrazolopyrimidine Analog.

### BACKGROUND OF THE INVENTION

Mammalian Target of Rapamycin, mTOR, is a cell-signaling protein that regulates the response of tumor cells to nutrients and growth factors, as well as controlling tumor blood supply through effects on Vascular Endothelial Growth Factor, VEGF. Inhibitors of mTOR starve cancer cells and shrink tumors by inhibiting the effect of mTOR. All mTOR inhibitors bind to the mTOR kinase. This has at least two important effects. First, mTOR is a downstream mediator of the PI3K/Akt pathway. The PI3K/Akt pathway is thought to be over activated in numerous cancers and may account for the widespread response from various cancers to mTOR inhibitors. The over-activation of the upstream pathway would normally cause mTOR kinase to be over activated as well. However, in the presence of mTOR inhibitors, this process is blocked. The blocking effect prevents mTOR from signaling to downstream pathways that control cell growth. Over-activation of the PI3K/Akt kinase pathway is frequently associated with mutations in the PTEN gene, which is common in many cancers and may help predict what tumors will respond to mTOR inhibitors. The second major effect of mTOR inhibition is anti-angiogenesis, via the lowering of VEGF levels.

In lab tests, certain chemotherapy agents were found to be more effective in the presence of mTOR inhibitors. George, J. N., et al, Cancer Research, 61, 1527-1532, 2001. Additional lab results have shown that some rhabdomyosarcoma cells die in the presence of mTOR inhibitors. The complete functions of the mTOR kinase and the effects of mTOR inhibition are not completely understood.

Phosphatidylinositol (hereinafter abbreviated as “PI”) is one of the phospholipids in cell membranes. In recent years it has become clear that PI plays an important role also in intracellular signal transduction. It is well recognized in the art that especially PI (4,5) bisphosphate (PI(4,5)P2) is degraded into diacylglycerol and inositol (1,4,5) triphosphate by phospholipase C to induce activation of protein kinase C and intracellular calcium mobilization, respectively [M. J. Berridge et al., Nature, 312, 315 (1984); Y. Nishizuka, Science, 225, 1365 (1984)].

In the late 1980s, phosphatidylinositol-3 kinase (“PI3K”) was found to be an enzyme that phosphorylates the 3-position of the inositol ring of phosphatidylinositol [D. Whitman et al., Nature, 332, 664 (1988)].

When PI3K was discovered, it was originally considered to be a single enzyme. Recently however, it was clarified that a plurality of subtypes are present in PI3K. Three major classes of PI3Ks have now been identified on the basis of their in vitro substrate specificity [B. Vanhaesebroeck, Trend in Biol. Sci., 22, 267(1997)].

Substrates for class I PI3Ks are PI, PI(4)P and PI(4,5)P2. In these substrates, PI(4,5)P2 is the most advantageous substrate in cells. Class I PI3Ks are further divided into two groups, class Ia and class Ib, in terms of their activation mechanism. Class Ia PI3Ks, which include PI3K p110α, p110β and p110δ subtypes, are activated in the tyrosine kinase system. Class Ib PI3K is a p110γ subtype activated by a G protein-coupled receptor.

PI and PI(4)P are known as substrates for class II PI3Ks but PI(4,5)P2 is not a substrate for the enzymes of this class. Class II PI3Ks include PI3K C2α, C2β and C2γ subtypes, which are characterized by containing C2 domains at the C terminus, implying that their activity will be regulated by calcium ions.

The substrate for class III PI3Ks is PI only. A mechanism for activation of the class III PI3Ks is not clarified yet. Because each subtype has its own mechanism for the regulating activity, it is considered that the respective subtypes will be activated depending on their respective stimuli specific to each of them.

In the PI3K subtypes, the class Ia subtype has been most extensively investigated to date. The three subtypes of class Ia are hetero dimers of a catalytic 110 kDa subunit and regulatory subunits of 85 kDa and 55 kDa. The regulatory subunits contain SH2 domains and bind to tyrosine residues phosphorylated by growth factor receptors with a tyrosine kinase activity or oncogene products, thereby inducing the PI3K activity of the p110 catalytic subunit. Thus, the class Ia subtypes are considered to be associated with cell proliferation and carcinogenesis. Furthermore, the class Ia PI3K subtypes bind to activated ras oncogene to express their enzyme activity. It has been confirmed that the activated ras oncogene is present in many cancers, suggesting a role of class Ia PI3Ks in carcinogenesis.

As explained above, mTOR inhibitors and PI3K inhibitors are expected to be novel types of medicaments useful against cell proliferation disorders, especially as carcinostatic agents. Thus, it would be advantageous to have new mTOR inhibitors and PI3K inhibitors as potential treatment regimens for mTOR- and PI3K-related diseases. The instant invention is directed to these and other important ends.

### SUMMARY OF THE INVENTION

In one aspect, the invention provides compounds of the Formula (I):

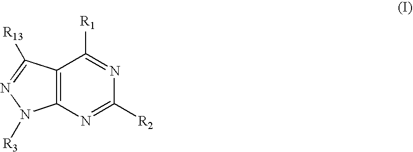

and pharmaceutically acceptable salts, hydrates, and solvates thereof, wherein

R₁ is

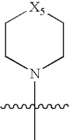

wherein X₅ is —O—, —S(O)ₙ, —CH₂—, —CH(OH)—, —C(O)—, —NH—, —N(optionally substituted alkyl)-, or the moiety

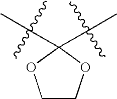

wherein any one or more of the ring hydrogen atoms of

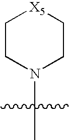

can independently be substituted with C₁-C₃ alkyl, C₁-C₃ alkenyl, C₁-C₃ alkynyl, C₁-C₃ alkoxy, C₁-C₃ acyl, C₁-C₃ alkoxycarbonyl, amino(C₁-C₆alkyl), hydroxyl, ═O, fluorine, or —CN and wherein n is an integer from 0 to 2;

R₂ is optionally substituted C₁-C₁₀alkyl, optionally substituted C₂-C₁₀alkenyl, optionally substituted C₂-C₁₀alkynyl, optionally substituted C₆-C₁₄aryl, optionally substituted C₁-C₉heteroaryl, optionally substituted C₆-C₁₄arylurea, optionally substituted C₆-C₁₄arylcarbamate, optionally substituted C₆-C₁₄arylthiourea, optionally substituted —HC═CH-aryl, or optionally substituted —HC═CH-heteroaryl;

R₃ is hydrogen, optionally substituted C₁-C₁₀alkyl, optionally substituted C₂-C₁₀alkenyl, optionally substituted C₂-C₁₀alkynyl, optionally substituted acyl, optionally substituted C₆-C₁₄aryl, optionally substituted C₁-C₉heteroaryl, heterocyclyl(C₁-C₆alkyl), C₁-C₆hydroxylalkyl, C₁-C₆alkylcarboxy, alkylamino-alkoxy, C₁-C₆perfluoroalkyl, —S(O)_{q}—(C₁-C₆alkyl) wherein the C₁-C₆alkyl of —S(O)_{q}—(C₁-C₆alkyl) can be optionally substituted, —S(O)_{q}-aryl wherein the C₆-C₁₄aryl of —S(O)_{q}-aryl can be optionally substituted, optionally substituted C₃-C₈carbocycle, optionally substituted 6- to 10-membered bicyclic carbocycle, 4- to 7-membered monocyclic C₁-C₆heterocycle, nitrogen containing 4- to 7-membered monocyclic C₁-C₆heterocycle, 6- to 10-membered bicyclic heterocycle, or nitrogen-containing 6- to 10-membered bicyclic heterocycle;

R₁₃ is hydrogen, halogen, optionally substituted C₁-C₆alkyl, C₁-C₆ alkene, C₁-C₆ alkyne, optionally substituted C₆-C₁₄aryl, or optionally substituted C₁-C₉heteroaryl; and

q is 1 or 2.

In another aspect, the invention provides compounds of Formula (II):

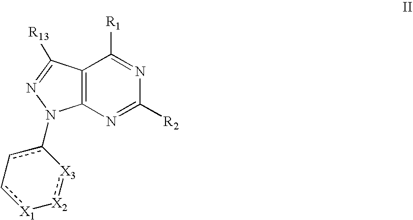

and pharmaceutically acceptable salts, hydrates, and solvates thereof, wherein

R₁ is

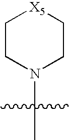

wherein X₅ is —O—, —S(O)ₙ,—, —CH₂—, —CH(OH)—, —C(O)—, —NH—, —N(optionally substituted alkyl)-, or the moiety

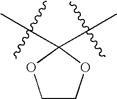

wherein any one or more of the ring hydrogen atoms of

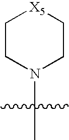

can independently be substituted with C₁-C₃ alkyl, C₁-C₃ alkenyl, C₁-C₃ alkynyl, C₁-C₃ alkoxy, C₁-C₃acyl, C₁-C₃ alkoxycarbonyl, amino(C₁-C₆alkyl), hydroxyl, ═O, or —CN and wherein n is an integer from 0 to 2;

R₂ is optionally substituted C₁-C₆alkyl, optionally substituted C₂-C₁₀ alkenyl, optionally substituted C₂-C₁₀ alkynyl, optionally substituted C₆-C₁₄aryl, optionally substituted C₁-C₉heteroaryl, optionally substituted C₆-C₁₄arylurea, optionally substituted C₆-C₁₄arylcarbamate, optionally substituted —HC═CH-aryl, or optionally substituted —HC═CH-heteroaryl;

X₁ and X₂ are each independently —N(R₄)—, —CH(OH)—, —C(O)—, —O—, —CH—, —CH₂—, —S(O)ₙ, or

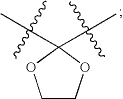

X₃ is —O—, or optionally substituted —CH₂—; with the proviso that X₁, X₂ and X₃ are not all heteroatoms simultaneously;

R₄ is —H, optionally substituted C₁-C₆alkyl, optionally substituted —C(O)alkyl, optionally substituted —C(O)alkoxy, optionally substituted —C(O)NR₅R₆, —SO₂R₁₅, —C(O)OC₂-C₁₀alkyne, —C(═S)—NHalkyl, —C(═O)—S-alkyl, optionally substituted C₆-C₁₄aryl, optionally substituted C₁-C₉heteroaryl, optionally substituted heterocycle, or the structure

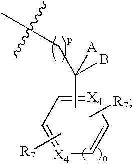

R₅ and R₆ are independently hydrogen, optionally substituted —C₁-C₆alkyl, optionally substituted C₂-C₆ alkenyl, optionally substituted C₂-C₆ alkynyl, optionally substituted C₆-C₁₄aryl, optionally substituted C₁-C₉heteroaryl, —C(O)·NRₐR_{b}, —C(O)—R₁₅, —SO₂—R₁₅, C₁-C₆perfluoroalkyl, or R₅ and R₆ can be taken together with the nitrogen to which they are attached to form a nitrogen containing 3 to 7 membered monocyclic C₁-C₆heterocycle, optionally having one or two of the methylene units of the ring substituted with —N—R₈, O, or S(O)ₙ, wherein n is 0, 1, or 2; provided that when X₄ is not —CH—, the ring is not connected to the structure of Formula II through X₄;

each R₇ is independently —H, —OH, halogen, optionally substituted alkyl, optionally substituted alkoxy, optionally substituted acyl, optionally substituted amine, optionally substituted amide, or —CN;

R₈ is optionally substituted C₁-C₆alkyl, optionally substituted —C(O)—C₁-C₆alkyl, —C(O)NR₅R₆, or —C(O)OC₁-C₆alkyl;

R₁₃ is hydrogen, halogen, optionally substituted C₁-C₆alkyl, C₁-C₆ alkene, C₁-C₆ alkyne, optionally substituted C₆-C₁₄aryl, or optionally substituted C₁-C₉heteroaryl;

R₁₅ is hydrogen, optionally substituted C₁-C₆alkyl, optionally substituted C₃-C₈carbocycle, optionally substituted C₆-C₁₄aryl, optionally substituted C₁-C₉heteroaryl, optionally substituted (C₁-C₆alkyl)amino, or optionally substituted (C₆-C₁₄aryl)amino, with the proviso that when R₁₅ is in —SO₂—R₁₅, R₁₅ is not —H;

Rₐ and R_{b} are independently hydrogen, optionally substituted —C₁-C₆alkyl, optionally substituted C₂-C₆ alkenyl, optionally substituted C₂-C₆ alkynyl, —C(O)—R₁₅, —SO₂—R₁₅, or Rₐ and R_{b} can be taken together with the nitrogen to which they are attached to form a nitrogen containing 3- to 7-membered monocyclic C₁-C₆heterocycle, optionally having one or two of the methylene units of the ring substituted with —N—R₈, O, or S(O)ₙ, wherein n is 0, 1, or 2;

A and B are each independently hydrogen, halogen, —C₁-C₃ alkyl, or A and B are taken together to form a carbonyl or carbocycle;

each X₄ is independently —CH—, —N—, —O—, —S—, or —N(O⁻)—;

o is 0 or 1;

p is 0 or 1; and

[symbol] represents an optional double bond with the proviso that the ring contains either zero or three double bonds.

In another aspect, the invention provides compounds of Formula III:

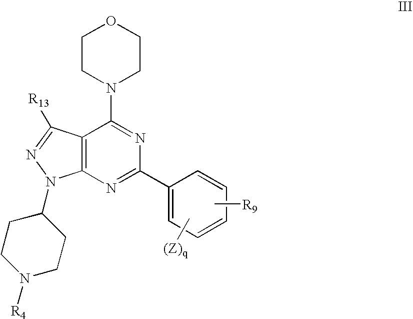

and pharmaceutically acceptable salts, hydrates, and solvates thereof, wherein

R₄ is —H, optionally substituted C₁-C₆alkyl, optionally substituted —C(O)alkyl, optionally substituted —C(O)alkoxy, optionally substituted —C(O)NR₅R₆, —SO₂R₁₅, —C(O)OC₂-C₁₀alkyne, —C(═S)-NHalkyl, —C(═O)—S-alkyl, optionally substituted C₆-C₁₄aryl, optionally substituted C₁-C₉heteroaryl, optionally substituted heterocycle, or the structure

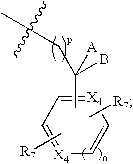

R₅ and R₆ are independently hydrogen, optionally substituted —C₁-C₆alkyl, optionally substituted C₂-C₆ alkenyl, optionally substituted C₂-C₆ alkynyl, optionally substituted C₆-C₁₄aryl, optionally substituted C₁-C₉heteroaryl, —C(O)—NRₐR_{b}, —C(O)—R₁₅, —SO₂—R₁₅, C₁-C₆perfluoroalkyl, or R₅ and R₆ can be taken together with the nitrogen to which they are attached to form a nitrogen containing 3 to 7 membered monocyclic C₁-C₆heterocycle, optionally having one or two of the methylene units of the ring substituted with —N—R₈, O, or S(O), wherein n is 0, 1, or 2; provided that when X₄ is not —CH—, the ring is not connected to the structure of Formula II through X₄;

each R₇ is independently —H, —OH, halogen, optionally substituted alkyl, optionally substituted alkoxy, optionally substituted acyl, optionally substituted amine, optionally substituted amide, or —CN;

R₈ is optionally substituted C₁-C₆alkyl, optionally substituted —C(O)—C₁-C₆alkyl, —C(O)NR₅R₆, or —C(O)OC₁-C₆alkyl;

R₉ is —OH, —NHC(O)NR₁₀R₁₁, —NHC(O)OR₁₂, —NH(SO₂)NHalkyl, —NH(SO₂)NHaryl, —NHC(S)—NHalkyl, —N═C(Salkyl) Halkyl), or —N(H)—C(═N—(CN))—(Oaryl);

R₁₀ and R₁₁ are each independently —H, —OH, optionally substituted C₁-C₆alkoxy, optionally substituted C₆-C₁₄aryl, optionally substituted C₁-C₉heteroaryl, optionally substituted —C₃-C₈carbocycle, or optionally substituted —C₁-C₆alkyl; or R₁₀ and R₁₁ when taken together with the nitrogen to which they are attached—form a 3- to 7- membered nitrogen containing heterocycle wherein up to two of the carbon atoms of the heterocycle may be substituted with —N(R₁₅)—, —O—, or —S(O)ₙ;

R₁₂ is optionally substituted —C₁-C₆alkyl or C₆-C₁₄aryl;

R₁₃ is hydrogen, halogen, optionally substituted C₁-C₆alkyl, C₁-C₆ alkene, C₁-C₆ alkyne, optionally substituted C₆-C₁₄aryl, or optionally substituted C₁-C₉heteroaryl;

R₁₅ is hydrogen, optionally substituted C₁-C₆alkyl, optionally substituted C₃-C₈carbocycle, optionally substituted C₆-C₁₄aryl, optionally substituted C₁-C₉heteroaryl, optionally substituted (C₁-C₆alkyl)amino, or optionally substituted (C₆-C₁₄aryl)amino with the proviso that when R₁₅ is in-—SO₂—R₁₅, R₁₅ is not —H;

Rₐ and R_{b} are independently hydrogen, optionally substituted —C₁-C₆alkyl, optionally substituted C₂-C₆ alkenyl, optionally substituted C₂-C₆ alkynyl, —C(O)—R₁₅, —SO₂—R₁₅, or Rₐ and R_{b} can be taken together with the nitrogen to which they are attached to form a nitrogen containing 3- to 7-membered monocyclic C₁-C₆heterocycle, optionally having one or two of the methylene units of the ring substituted with —N—R₈, O, or S(O) n, wherein n is 0, 1, or 2;

A and B are each independently hydrogen, halogen, —C₁-C₃ alkyl, or A and B are taken together to form a carbonyl or carbocycle;

each X₄ is independently —CH—, —N—, —O—, —S—, or —N⁺(O⁻)—;

o is 0 or 1;

p is 0 or 1; and

Z is halogen, alkyl, or alkoxy.

In another aspect, the invention provides compounds of Formula IIIa:

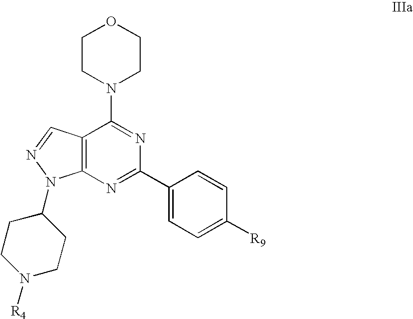

wherein

R₄ is optionally substituted —C(O)alkoxy, optionally substituted —C(O)NR₅R₆, —C(O)OC₂-C₁₀alkyne,

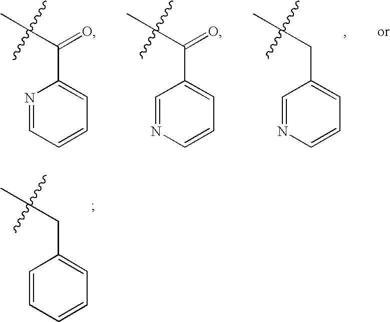

R₅ and R₆ are independently hydrogen, optionally substituted —C₁-C₆alkyl, optionally substituted C₆-C₁₄aryl, optionally substituted C₁-C₉heteroaryl, or R₅ and R₆ can be taken together with the nitrogen to which they are attached to form a nitrogen containing 3 to 7 membered monocyclic C₁-C₆heterocycle, optionally having one or two of the methylene units of the ring substituted with —N—R₈, O, or S(O), wherein n is 0, 1, or 2;

R₈ is optionally substituted C₁-C₆alkyl, or optionally substituted —C(O)—C₁-C₆alkyl;

R₉ is —NHC(O)NR₁₀R₁₁, or —NHC(O)OR₁₂;

R₁₀ and R₁₁ are each independently —H, —OH, optionally substituted C₁-C₆alkoxy, optionally substituted C₆-C₁₄aryl, optionally substituted C₁-C₉heteroaryl, optionally substituted —C₃-C₈carbocycle, or optionally substituted —C₁-C₆alkyl; or R₁₀ and R₁₁ when taken together with the nitrogen to which they are attached—form a 3- to 7- membered nitrogen containing heterocycle wherein up to two of the carbon atoms of the heterocycle may be substituted with —N(R₁₅)—, —O—, or —S(O)ₙ;

R₁₂ is optionally substituted -C₁-C₆alkyl or C₆-C₁₄aryl; and

R₁₅ is hydrogen, optionally substituted C₁-C₆alkyl, optionally substituted C₃-C₈carbocycle, optionally substituted C₆-C₁₄aryl, optionally substituted C₁-C₉heteroaryl, optionally substituted (C₁-C₆alkyl)amino, or optionally substituted (C₆-C₁₄aryl)amino.

In another aspect, the invention provides compounds of Formula Ia:

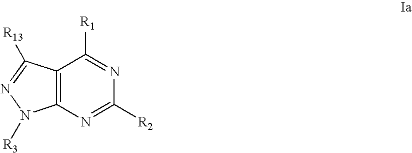

or a pharmaceutically acceptable salt or tautomer thereof,
R₁ is:

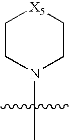

X₅ is —O—, —CH₂—O—, or —S(O)ₙ—, and any one or more of the ring hydrogen atoms of R₁ can independently be replaced with C₁-C₆alkyl, C₂-C₆alkenyl, C₂-C₆alkynyl, C₁-C₃alkoxy, C₁-C₃acyl, C₁-C₃alkoxycarbonyl, amino(C₁-C₆alkyl), hydroxyl, fluorine, or —CN, where any two hydrogen atoms attached to the same carbon atom of R₁ can be replaced by an oxygen atom, the oxygen atom taken together with the carbon to which it is attached, forming a carbonyl (C═O), and wherein n is an integer from 0 to 2;
R₂ is
   (a) C₆-C₁₄aryl optionally substituted with from 1 to 3 substituents independently selected from:
      (i) halogen,
      (ii) C₁-C₆alkyl optionally substituted with from 1 to 3 substituents independently selected from halogen, heterocycle, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl,
      (iii) C₁-C₆alkoxy optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl,
      (iv) C₁-C₆alkoxycarbonyl,
      (v) C₂-C₆alkenyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl,
      (vi) C₂-C₆alkynyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₁₆alkyl), —C(O)(C₁-C₆alkyl), C₆-C₁₄aryl, C₁-C₉heteroaryl, and C₃-C₈cycloalkyl,
      (vii) C₃-C₈cycloalkyl optionally substituted with from 1 to 3 substituents independently selected from C₁-C₆alkyl, halo, halo(C₁-C₆alkyl)-, hydroxyl, —O—C₁-C₆alkyl, —NH₂, amino(C₁-C₆alkyl)-, (C₁-C₆alkyl)amino-, di(C₁-C₆alkyl)amino-, —COOH, —C(O)O—(C₁-C₆alkyl), —OC(O)—(C₁-C₆alkyl), (C₁-C₆alkyl)carboxyamido-, —C(O)NH₂, carboxyamidoalkyl- and —NO₂,
      (viii) C₆-C₁₄aryl optionally substituted with from 1 to 3 substituents independently selected from C₁-C₆alkyl, halo, halo(C₁-C₆alkyl)-, hydroxyl, C₁-C₆hydroxylalkyl, —NH₂, amino(C₁-C₆alkyl)-, (C₁-C₆alkyl)amino-, di(C₁-C₆alkyl)amino-, —COOH, —C(O)O—(C₁-C₆alkyl), —OC(O)—(C₁-C₆alkyl), (C₁-C₆alkyl)carboxyamido-, —C(O)NH₂, (C₁-C₆alkyl)N-alkylamido-, and —NO₂,
      (ix) C₁-C₉heteroaryl optionally substituted with from 1 to 3 substituents independently selected from C₁-C₆alkyl, halo, halo(C₁-C₆alkyl)-, hydroxyl, C₁-C₆hydroxylalkyl, —NH₂, amino(C₁-C₆alkyl)-, (C₁-C₆alkyl)amino-, di(C₁-C₆alkyl)amino-, —COOH, —C(O)O—(C₁-C₆alkyl), —OC(O)—(C₁-C₆alkyl), (C₁-C₆alkyl)carboxyamido-, —C(O)NH₂, (C₁-C₆alkyl)N-alkylamido-, and —NO₂,
      (x) C₁-C₆perfluoroalkyl-,
      (xi) hydroxyl,
      (xii) NR¹⁶R¹⁷,
      (xiii) NO₂,
      (xiv) CN,
      (xv) CO₂H,
      (xvi) CF₃,
      (xvii) CF₃O,
      (xviii) C₁-C₆alkylthio,
      (xix) —SO₂NR¹⁶R¹⁷,
      (xx) —O—C(O)NR¹⁶R¹⁷,
      (xxi) —C(O)NR¹⁶R¹⁷,
      (xxii) NR¹⁷C(O)R¹⁶,
      (xxiii) N(C₁-C₆alkyl)C(O)R¹⁶,
      (xxiv) —NHC(O)NR¹⁶R¹⁷,
      (xxv) —NHC(O)NHNR¹⁶R¹⁷,
      (xxvi) —NHC(O)OR¹⁸,
      (xxvii) —NHC(O)NHOR¹⁶,
      (xxviii) —NH(SO₂)NH—(C₁-C₆alkyl),
      (xxix) —NH(SO₂)—(C₁-C₆alkyl),
      (xxx) —NH(SO₂)NH—C₆-C₁₄aryl,
      (xxxi) —NHC(S)—NH—C₁-C₆alkyl,
      (xxxii) —N═C(S—C₁-C₆alkyl)(NH—C₁-C₆alkyl),
      (xxxiii) —S(O)ₚ—C₆-C₁₄aryl,
      (xxxiv) —S(O)ₚ—C₁-C₉heteroaryl,
      (xxxv) —N(H)—C(═N—(CN))—(NR¹⁶R¹⁷), and
      (xxxvi) —N(H)—C(═N—(CN))—(O—R¹⁶);
   (b) C₁-C₉heteroaryl optionally substituted with from 1 to 3 substituents independently selected from:
      (i) halogen,
      (ii) C₁-C₆alkyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl,
      (iii) C₁-C₆alkoxy optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl,
      (iv) C₁-C₆alkoxycarbonyl,
      (v) C₂-C₆alkenyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl,
      (vi) C₂-C₆alkynyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl,
      (vii) C₃-C₈cycloalkyl optionally substituted with from 1 to 3 substituents independently selected from C₁-C₆alkyl, halo, halo(C₁-C₆alkyl)-, hydroxyl, —O—C₁-C₆alkyl, —NH₂, amino(C₁-C₆alkyl)-, (C₁-C₆alkyl)amino-, di(C₁-C₆alkyl)amino-, —COOH, —C(O)O—(C₁-C₆alkyl), —OC(O)—(C₁-C₆alkyl), (C₁-C₆alkyl)carboxyamido-, —C(O)NH₂, carboxyamidoalkyl- and —NO₂,
      (viii) C₆-C₁₄aryl optionally substituted with from 1 to 3 substituents independently selected from C₁-C₆alkyl, halo, halo(C₁-C₆alkyl)-, hydroxyl, C₁-C₆hydroxylalkyl, —NH₂, amino(C₁-C₆alkyl)-, (C₁-C₆alkyl)amino-, di(C₁-C₆alkyl)amino-, —COOH, —C(O)O—(C₁-C₆alkyl), —OC(O)—(C₁-C₆alkyl), (C₁-C₆alkyl)carboxyamido-, —C(O)NH₂, (C₁-C₆alkyl)N-alkylamido-, and —NO₂,
      (ix) C₁-C₉heteroaryl optionally substituted with from 1 to 3 substituents independently selected from C₁-C₆alkyl, halo, halo(C₁-C₆alkyl)-, hydroxyl, C₁-C₆hydroxylalkyl, —NH₂, amino(C₁-C₆alkyl)-, (C₁-C₆alkyl)amino-, di(C₁-C₆alkyl)amino-, —COOH, —C(O)O—(C₁-C₆alkyl), —OC(O)—(C₁-C₆alkyl), (C₁-C₆alkyl)carboxyamido-, —C(O)NH₂, (C₁-C₆alkyl)N-alkylamido-, and —NO₂,
      (x) C₁-C₆perfluoroalkyl-,
      (xi) hydroxyl,
      (xii) NR¹⁶R¹⁷,
      (xiii) NO₂,
      (xiv) CN,
      (xv) CO₂H,
      (xvi) CF₃,
      (xvii) CF₃O,
      (xviii) C₁-C₆alkylthio,
      (xix) —SO₂NR¹⁶R¹⁷,
      (xx) —C(O)NR¹⁶R¹⁷,
      (xxi) NR¹⁷C(O)R¹⁶,
      (xxii) —NHC(O)NR¹⁶R¹⁷,
      (xxiii) —NHC(O)NHNR¹⁶R¹⁷,
      (xxiv) —NHC(O)OR¹⁸,
      (xxv) —NHC(O)NHOR¹⁶,
      (xxvi) —NH(SO₂)NH—C₁-C₆alkyl,
      (xxvii) —NH(SO₂)NH—C₆-C₁₄aryl,
      (xxviii) —NHC(S)—NH—C₁-C₆alkyl,
      (xxix) —N═C(S—C₁-C₆alkyl)(NH—C₁-C₆alkyl),
      (xxx) —S(O)ₚ—C₆-C₁₄aryl,
      (xxxi) —S(O)ₚ—C₁-C₉heteroaryl,
      (xxxii) —N(H)—C(═N—(CN))—(NR¹⁶R¹⁷),
      (xxxiii) —N(H)—C(═N—(CN))—(O—R¹⁶), and
      (xxxiv) —N(H)—C(═N—(CN))—(O—C₆-C₁₄aryl);
   (c) —HC═CH—C₆-C₁₄aryl;
   (d) heterocycle attached by a ring carbon atom of the heterocycle; or,
   (e) —HC═CH—C₁-C₉heteroaryl;
R¹⁶ and R¹⁷ are each independently selected from
a) H;
b) C₁-C₆alkoxy;
c) C₁-C₆perfluoroalkyl;
d) C₆-C₁₄aryl optionally substituted with from 1 to 3 substituents independently selected from:
   (i) C₁-C₆alkyl wherein the C₁-C₆alkyl is optionally substituted with:
      A) heterocycle optionally substituted with C₁-C₆alkyl,
      B) NH₂—,
      C) (C₁-C₆alkyl)amino-,
      D) di(C₁-C₆alkyl)amino-,
   (ii) C₁-C₆alkoxy,
   (iii) halo,
   (iv) halo(C₁-C₆alkyl)-,
   (v) hydroxyl,
   (vi) C₁-C₆hydroxylalkyl,
   (vii) heterocycle optionally substituted with C₁-C₆alkyl,
   (viii) NH₂—,
   (ix) amino(C₁-C₆alkyl)-,
   (x) (C₁-C₆alkyl)amino-,
   (xi) di(C₁-C₆alkyl)amino-,
   (xii) C₁-C₆alkoxy-C₁-C₆alkylene-NH—C₁-C₆alkylene-,
   (xiii) C₁-C₆hydroxylalkyl-NH—C₁-C₆alkylene-,
   (xiv) amino(C₁-C₆alkyl)-NH—C₁-C₆alkylene-,
   (xv) di(C₁-C₆alkyl)amino-C₁-C₆alkylene-NH—C₁-C₆alkylene-,
   (xvi) C₁-C₆hydroxylalkyl-NH—,
   (xvii) amino(C₁-C₆alkyl)-NH—,
   (xviii) —COOH,
   (xix) —C(O)O—(C₁-C₆alkyl),
   (xx) —OC(O)—(C₁-C₆alkyl),
   (xxi) (C₁-C₆alkyl)carboxyamido-,
   (xxii) —C(O)NH₂,
   (xxiii) (C₁-C₆alkyl)N-alkylamido-,
   (xxiv) C₁-C₆alkoxy,
   (xxv) or —NO₂;
e) C₁-C₉heteroaryl optionally substituted with from 1 to 3 substituents independently selected from:
   (i) C₁-C₆alkyl,
   (ii) C₁-C₆alkoxy,
   (iii) halo,
   (iv) halo(C₁-C₆alkyl)-,
   (v) hydroxyl,
   (vi) C₁-C₆hydroxylalkyl,
   (vii) NH₂—,
   (viii) amino(C₁-C₆alkyl)-,
   (ix) (C₁-C₆alkyl)amino-,
   (x) di(C₁-C₆alkyl)amino-,
   (xi) —COOH,
   (xii) —C(O)O—(C₁-C₆alkyl),
   (xiii) —OC(O)—(C₁-C₆alkyl),
   (xiv) (C₁-C₆alkyl)carboxyamido-,
   (xv) —C(O)NH₂,
   (xvi) (C₁-C₆alkyl)N-alkylamido-, and
   (xvii) —NO₂;
f) C₃-C₈cycloalkyl optionally substituted with from 1 to 3 substituents independently selected from:
   (i) C₁-C₆alkyl,
   (ii) C₁-C₆alkoxy,
   (iii) halo,
   (iv) halo(C₁-C₆alkyl)-,
   (v) hydroxyl,
   (vi) —O—C₁-C₆alkyl,
   (vii) —NH₂,
   (viii) -amino(C₁-C₆alkyl),
   (ix) (C₁-C₆alkyl)amino-,
   (x) di(C₁-C₆alkyl)amino-,
   (xi) —COOH,
   (xii) —C(O)O—(C₁-C₆alkyl),
   (xiii) —OC(O)—(C₁-C₆alkyl),
   (xiv) (C₁-C₆alkyl)carboxyamido-,
   (xv) —C(O)NH₂,
   (xvi) carboxyamidoalkyl-, and
   (xvii) —NO₂;
g) C₁-C₆alkyl optionally substituted with from 1 to 3 substituents independently selected from:
   (i) halogen,
   (ii) —NH₂,
   (iii) —NH(C₁-C₆alkyl),
   (iv) —N(C₁-C₆alkyl)(C₁-C₆alkyl),
   (v) —CO₂H,
   (vi) hydroxyl,
   (vii) heterocycle,
   (viii) C₁-C₆alkoxy,
   (ix) C₆-C₁₄aryl wherein the C₆-C₁₄aryl is optionally substituted with di(C₁-C₆alkyl)amino-,
   (x) C₁-C₉heteroaryl,
   (xi) and C₃-C₈cycloalkyl;
h) C₂-C₆alkenyl optionally substituted with from 1 to 3 substituents independently selected from:
   (i) halogen,
   (ii) —NH₂,
   (iii) —NH(C₁-C₆alkyl),
   (iv) —N(C₁-C₆alkyl)(C₁-C₆alkyl),
   (v) hydroxyl,
   (vi) C₁-C₆alkoxy,
   (vii) C₁-C₆alkyl,
   (viii) C₆-C₁₄aryl,
   (ix) C₁-C₉heteroaryl,
   (x) and C₃-C₈cycloalkyl;
i) C₃-C₆alkynyl optionally substituted with from 1 to 3 substituents independently selected from:
   (i) halogen,
   (ii) —NH₂,
   (iii) —NH(C₁-C₆alkyl),
   (iv) —N(C₁-C₆alkyl)(C₁-C₆alkyl),
   (v) hydroxyl,
   (vi) C₁-C₆alkoxy,
   (vii) C₁-C₆alkyl,
   (viii) C₆-C₁₄aryl,
   (ix) C₁-C₉heteroaryl,
   (x) and C₃-C₈cycloalkyl; and
j) heterocycle optionally substituted with from 1 to 3 substituents independently selected from:
   (i) C₁-C₆alkyl,
   (ii) C₆-C₁₄aryl,
   (iii) and C₁-C₉heteroaryl;

or R¹⁶ and R¹⁷ when taken together with the nitrogen to which they are attached can form a 3- to 7- membered nitrogen containing heterocycle wherein up to two of the carbon atoms of the heterocycle can be replaced with —N(H)—,—N(C₁-C₆alkyl), —O—, or —S(O)ₚ—;

R¹⁸ is
(a) C₁-C₆alkyl optionally substituted with from 1 to 3 substituents independently selected from:
   (i) halogen,
   (ii) —NH₂,
   (iii) —NH(C₁-C₆alkyl),
   (iv) —N(C₁-C₆alkyl)(C₁-C₆alkyl),
   (v) —N(C₁-C₃alkyl)C(O)(C₁-C₆alkyl),
   (vi) —NHC(O)(C₁-C₆alkyl),
   (vii) —NHC(O)H,
   (viii) —C(O)NH₂,
   (ix) —C(O)NH(C₁-C₆alkyl),
   (x) —C(O)N(C₁-C₆alkyl)(C₁-C₆alkyl),
   (xi) —CN,
   (xii) hydroxyl,
   (xiii) C₁-C₆alkoxy,
   (xiv) C₁-C₆alkyl,
   (xv) —C(O)OH,
   (xvi) —C(O)O(C₁-C₆alkyl),
   (xvii) —C(O)(C₁-C₆alkyl),
   (xviii) C₆-C₁₄aryl,
   (xix) heterocycle optionally substituted with C₁-C₆alkyl,
   (xx) C₁-C₉heteroaryl,
   (xxi) and C₃-C₈cycloalkyl;
(b) monocyclic C₁-C₆heterocycle optionally substituted with from 1 to 3 substituents independently selected from:
   (i) C₁-C₈acyl,
   (ii) C₁-C₆alkyl,
   (iii) heteroaryl(C₁-C₆alkyl),
   (iv) heterocyclyl(C₁-C₆alkyl),
   (v) (C₆-C₁₄aryl)alkyl,
   (vi) and (C₁-C₆alkoxy)carbonyl;
(c) or C₆-C₁₄aryl optionally substituted with from 1 to 3 substituents independently selected from:
   (i) C₁-C₆alkyl,
   (ii) halo,
   (iii) halo(C₁-C₆alkyl)-,
   (iv) hydroxyl,
   (v) C₁-C₆hydroxylalkyl,
   (vi) —NH₂,
   (vii) -amino(C₁-C₆alkyl),
   (viii) (C₁-C₆alkyl)amino-,
   (ix) di(C₁-C₆alkyl)amino-,
   (x) —COOH,
   (xi) —C(O)O—(C₁-C₆alkyl),
   (xii) —OC(O)—(C₁-C₆alkyl),
   (xiii) (C₁-C₆alkyl)carboxyamido-,
   (xiv) —C(O)NH₂,
   (xv) (C₁-C₆alkyl)N-alkylamido-, and
   (xvi) —NO₂;
      each p is independently 1 or 2;
R₃ is
   (a) hydrogen;
   (b) C₁-C₁₀alkyl optionally substituted with from 1 to 3 substituents independently selected from:
      (i) C₁-C₆alkoxy,
      (ii) NH₂,
      (iii) (C₁-C₆alkyl)amino,
      (iv) di(C₁-C₆alkyl)amino,
      (v) heterocycle,

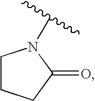

(vi) the group
(vii) C(O)NH₂,
(viii) CO₂H,
(ix) and (C₁-C₆alkoxy)carbonyl;
(c) C₂-C₁₀alkenyl;
(d) C₂-C₁₀alkynyl;
(e) C₁-C₈acyl;
(f) C₆-C₁₄aryl;
(g) C₁-C₉heteroaryl;
(h) C₁-C₆hydroxylalkyl;
(i) C₁-C₆alkylcarboxy;
(j) C₁-C₆perfluoroalkyl;
(k) —S(O)_{q}—(C₁-C₆alkyl);
(l) —S(O)_{q}-aryl;
(m) C₃-C₈carbocycle optionally substituted with from 1 to 3 substituents independently selected from:
   (i) C₁-C₆alkoxy,
   (ii) hydroxyl,
   (iii) heterocycle,
   (iv) (C₁-C₆alkoxy)-(C₆-C₁₄aryl)-NH—,
   (v) NH₂,
   (vi) (C₁-C₆alkyl)amino,
   (vii) di(C₁-C₆alkyl)amino,
   (viii) CO₂H,
   (ix) and (C₁-C₆alkoxy)carbonyl;

or two hydrogen atoms on the same carbon atom of the C₃-C₈carbocycle can be replaced by an oxygen atom, the oxygen atom taken together with the carbon to which it is attached forming a carbonyl (C═O) group, or two hydrogen atoms on the same carbon atom of the C₃-C₈carbocycle can be replaced by an alkylenedioxy group so that the alkylenedioxy group, when taken together with the carbon atom to which it is attached, forms a 5- to 7-membered heterocycle containing two oxygen atoms;
(n) 6- to 10-membered bicyclic carbocycle;
(o) monocyclic C₁-C₆heterocycle optionally substituted with from 1 to 3 substituents independently selected from:
   (i) C₁-C₈acyl, wherein the C₁-C₈acyl is optionally substituted with from 1 to 3 substituents independently selected from:
      A) hydroxyl,
      B) CN,
      C) C₁-C₆alkoxy,
      D) C₁-C₆alkyl,
      E) C₁-C₈acyl,
      F) NH₂,
      G) (C₁-C₆alkyl)amino,
      H) di(C₁-C₆alkyl)amino,
      I) CO₂H,
      J) (C₁-C₆alkoxy)carbonyl,
      K) C₁-C₆perfluoroalkyl,
      L) and halogen,
   (ii) C₁-C₆alkyl optionally substituted with from 1 to 3 substituents independently selected from:
      A) C₃-C₈cycloalkyl,
      B) C₁-C₆alkoxy,
      C) C₁-C₈acyl,
      D) CN,
      E) (C₁-C₆alkoxy)carbonyl,
      F) CO₂H,
      G) hydroxyl,
      H) C₁-C₉heterocycle, and
      I) H₂NC(O)—,
   (iii) C₁-C₆perfluoroalkyl,
   (iv) C₂-C₆alkenyl,
   (v) heteroaryl(C₁-C₆alkyl) wherein the ring portion of the heteroaryl(C₁-C₆alkyl) group is optionally substituted with from 1 to 3 substituents independently selected from:
      A) C₁-C₆alkylC(O)NH—,
      B) C₁-C₆alkoxy,
      C) halogen,
      D) NH₂,
      E) and C₁-C₆alkyl,
   (vi) (C₆-C₁₄aryl)alkyl, wherein the ring portion of the (C₆-C₁₄aryl)alkyl group is optionally substituted with 1 to 3 substituents independently selected from:
      A) halogen,
      B) C₁-C₆alkyl,
      C) NH₂,
      D) (C₁-C₆alkyl)amino,
      E) di(C₁-C₆alkyl)amino,
      F) hydroxyl,
      G) C₁-C₆alkoxy,
      H) C₁-C₈acyl,
      I) and C₁-C₉heteroaryl,
   (vii) HC(O)—,
   (viii) C₁-C₆perfluoroalkyl,
   (ix) —S(O)_{q}—(C₁-C₆alkyl),
   (x) —S(O)_{q}-aryl,
   (xi) R¹⁹R²⁰NC(O),
   (xii) (C₁-C₉heteroaryl)-NH—C(S)—,
   (xiii) (C₁-C₆alkyl)-NH—C(S)—,
   (xiv) (C₁-C₆alkyl)-S—C(O)—,
   (xv) (C₆-C₁₄aryloxy)carbonyl,
   (xvi) (C₂-C₆alkenyloxy)carbonyl,
   (xvii) (C₂-C₆alkynyloxy)carbonyl,
   (xviii) and (C₁-C₆alkoxy)carbonyl optionally substituted with from 1 to 3 substituents independently selected from:
      A) C₁-C₆alkoxy,
      B) halogen,
      C) C₆-C₁₄aryl,
      D) NH₂,
      E) (C₁-C₆alkyl)amino-,
      F) di(C₁-C₆alkyl)amino-,
      G) and C₁-C₆alkyl;
(p) or bicyclic C₁-C₉heterocycle;
R¹⁹ and R²⁰ are each independently:
   (a) H;
   (b) C₁-C₆alkyl optionally substituted with a substituent selected from:
      (i) C₁-C₆alkylC(O)NH—,
      (ii) NH₂,
      (iii) (C₁-C₆alkyl)amino,
      (iv) or di(C₁-C₆alkyl)amino;
   (c) C₃-C₈cycloalkyl;
   (d) C₆-C₁₄aryl optionally substituted with a substituent selected from:
      (i) halogen,
      (ii) and monocyclic C₁-C₆heterocycle wherein the monocyclic C₁-C₆heterocycle is optionally substituted with (C₁-C₆alkoxy)carbonyl;
   (e) C₁-C₉heteroaryl;
   (f) heteroaryl(C₁-C₆alkyl);
   (g) heterocyclyl(C₁-C₆alkyl);
   (h) (C₆-C₁₄aryl)alkyl, wherein the chain portion of the (C₆-C₁₄aryl)alkyl group is optionally substituted with a hydroxyl;
   (i) or monocyclic C₁-C₆heterocycle optionally substituted with a (C₁-C₆alkoxy)carbonyl;
   or R¹⁹ and R²⁰ when taken together with the nitrogen to which they are attached optionally form a 3- to 7- membered nitrogen-containing heterocycle wherein up to two of the carbon atoms of the heterocycle are optionally replaced with —N(H)—, —N(C₁-C₆alkyl)-, —N(C₆-C₁₄aryl)-, or —O—, and wherein the nitrogen-containing heterocycle is optionally substituted with a C₁-C₆alkyl; C₆-C₁₄aryl, (C₁-C₆alkoxy)C(O)NH—, or C₁-C₉heterocycle;
R₁₃ is hydrogen, halogen, C₁-C₆alkyl, C₂-C₆alkenyl, C₂-C₆alkynyl, C₆-C₁₄aryl, or C₁-C₉heteroaryl; and where each C₁-C₆alkyl, C₂-C₆alkenyl, C₂-C₆alkynyl, C₆-C₁₄aryl, or C₁-C₉heteroaryl is optionally substituted with a C₁-C₆hydroxylalkyl, NH₂, (C₁-C₆alkyl)amino, or di(C₁-C₆alkyl)amino;
and, q is 1 or 2;
except that 4-(4-morpholinyl)-1-phenyl-6-[3-(trifluoromethyl)phenyl]-1H-pyrazolo[3,4-d]pyrimidine is excluded.

In another aspect, the invention provides compounds of Formula IIIb:

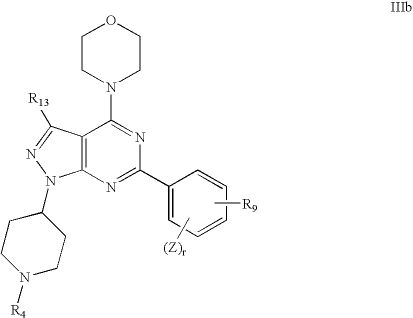

or a pharmaceutically acceptable salt or tautomer thereof, wherein
R₄ is selected from:
a) hydrogen;
b) C₁-C₈acyl, wherein the C₁-C₈acyl is optionally substituted with from 1 to 3 substituents independently selected from:
   (i) hydroxyl,
   (ii) CN,
   (iii) C₁-C₆alkoxy,
   (iv) C₁-C₆alkyl,
   (v) C₁-C₈acyl,
   (vi) NH₂,
   (vii) (C₁-C₆alkyl)amino,
   (viii) di(C₁-C₆alkyl)amino,
   (ix) CO₂H,
   (x) (C₁-C₆alkoxy)carbonyl,
   (xi) C₁-C₆perfluoroalkyl,
   (xii) and halogen;
c) C₁-C₆alkyl optionally substituted with from 1 to 3 substituents independently selected from:
   (i) C₃-C₈cycloalkyl,
   (ii) C₁-C₆alkoxy,
   (iii) C₁-C₈acyl,
   (iv) CN,
   (v) (C₁-C₆alkoxy)carbonyl,
   (vi) CO₂H,
   (vii) hydroxyl,
   (viii) C₁-C₉heterocycle, and
   (ix) H₂NC(O)—;
d) C₁-C₆perfluoroalkyl;
e) C₂-C₆alkenyl;
f) heteroaryl(C₁-C₆alkyl) wherein the ring portion of the heteroaryl(C₁-C₆alkyl) group is optionally substituted with from 1 to 3 substituents independently selected from:
   (i) C₁-C₆alkylC(O)NH—,
   (ii) C₁-C₆alkoxy,
   (iii) halogen,
   (iv) NH₂,
   (v) and C₁-C₆alkyl;
g) (C₆-C₁₄aryl)alkyl, wherein the ring portion of the (C₆-C₁₄aryl)alkyl group is optionally substituted with 1 to 3 substituents independently selected from:
   (i) halogen,
   (ii) C₁-C₆alkyl,
   (iii) NH₂,
   (iv) (C₁-C₆alkyl)amino,
   (v) di(C₁-C₆alkyl)amino,
   (vi) hydroxyl,
   (vii) C₁-C₆alkoxy,
   (viii) C₁-C₈acyl,
   (ix) and C₁-C₉heteroaryl;
h) HC(O)—;
i) C₁-C₆perfluoroalkyl;
j) —S(O)_{q}—(C₁-C₆alkyl);
k) —S(O)_{q}-aryl;
l) R¹⁹R²⁰NC(O);
m) (C₁-C₉heteroaryl)-NH—C(S)—;
n) (C₁-C₆alkyl)-NH—C(S)—;
o) (C₁-C₆alkyl)-S—C(O)—;
p) (C₆-C₁₄aryloxy)carbonyl;
q) (C₂-C₆alkenyloxy)carbonyl;
r) (C₂-C₆alkynyloxy)carbonyl;
s) and (C₁-C₆alkoxy)carbonyl optionally substituted with from 1 to 3 substituents independently selected from:
   (i) C₁-C₆alkoxy,
   (ii) halogen,
   (iii) C₆-C₁₄aryl,
   (iv) NH₂,
   (v) (C₁-C₆alkyl)amino-,
   (vi) di(C₁-C₆alkyl)amino-,
   (vii) and C₁-C₆alkyl;
R₉ is —OH, —NHC(O)NR₁₀R₁₁, —NHC(O)OR₁₂, —NH(SO₂)NHalkyl, —NH(SO₂)NHaryl, —NHC(S)—NHalkyl, —N═C(Salkyl) Halkyl), or —N(H)—C(═N—(CN))—(Oaryl);
R₁₀ and R₁₁ are each independently —H, —OH, C₁-C₆alkoxy, C₆-C₁₄aryl, C₁-C₉heteroaryl, C₃-C₈carbocycle, or C₁-C₆alkyl; or R₁₀ and R₁₁ when taken together with the nitrogen to which they are attached to form a 3- to 7- membered nitrogen containing heterocycle wherein up to two of the carbon atoms of the heterocycle may be substituted with —N(R₁₅)—, —O—, or —S(O)ₙ;
R₁₂ is C₁-C₆alkyl, C₁-C₆hydroxylalkyl, or C₆-C₁₄aryl;

R₁₃ is hydrogen, halogen, C₁-C₆alkyl, C₂-C₆alkenyl, C₂-C₆alkynyl, C₆-C₁₄aryl, or C₁-C₉heteroaryl; and where each C₁-C₆alkyl, C₂-C₆alkenyl, C₂-C₆alkynyl, C₆-C₁₄aryl, or C₁-C₉heteroaryl is optionally substituted with a C₁-C₆hydroxylalkyl, NH₂, (C₁-C₆alkyl)amino, or di(C₁-C₆alkyl)amino;
R₁₅ is hydrogen, C₁-C₆alkyl, C₃-C₈ carbocycle, C₆-C₁₄aryl, C₁-C₉heteroaryl, (C₁-C₆alkyl)amino, or (C₆-C₁₄aryl)amino;
n is 0, 1, or 2;
q is 1 or 2;
R¹⁹ and R²⁰ are each independently:
a) H;
b) C₁-C₆alkyl optionally substituted with a substituent selected from:
   (i) C₁-C₆alkylC(O)NH—,
   (ii) NH₂,
   (iii) (C₁-C₆alkyl)amino,
   (iv) or di(C₁-C₆alkyl)amino;
c) C₃-C₈cycloalkyl;
d) C₆-C₁₄aryl optionally substituted with a substituent selected from:
   (i) halogen,
   (ii) and monocyclic C₁-C₆heterocycle wherein the monocyclic C₁-C₆heterocycle is optionally substituted with (C₁-C₆alkoxy)carbonyl;
e) C₁-C₉heteroaryl;
f) heteroaryl(C₁-C₆alkyl);
g) heterocyclyl(C₁-C₆alkyl);
h) (C₆-C₁₄aryl)alkyl, wherein the chain portion of the (C₆-C₁₄aryl)alkyl group is optionally substituted with a hydroxyl;
i) or monocyclic C₁-C₆heterocycle optionally substituted with a (C₁-C₆alkoxy)carbonyl;
   or R¹⁹ and R²⁰ when taken together with the nitrogen to which they are attached optionally form a 3- to 7- membered nitrogen-containing heterocycle wherein up to two of the carbon atoms of the heterocycle are optionally replaced with —N(H)—, —N(C₁-C₆alkyl)-, —N(C₆-C₁₄aryl)-, or —O—, and wherein the nitrogen-containing heterocycle is optionally substituted with a C₁-C₆alkyl; C₆-C₁₄aryl, (C₁-C₆alkoxy)C(O)NH—, or C₁-C₉heterocycle;
   q is 1 or 2;
   r is 0 or 1; and
   Z is halogen, C₁-C₆alkyl, or C₁-C₆alkoxy.

In another aspect, the invention provides compounds compound of Formula IIIc:

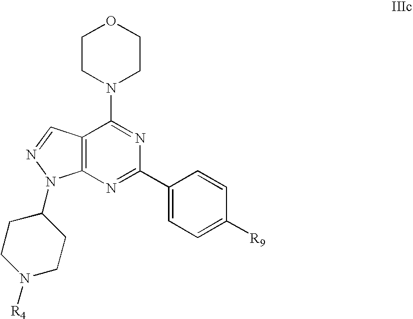

or a pharmaceutically acceptable salt or tautomer thereof, wherein
R₄ is selected from:
   a) hydrogen;
   b) C₁-C₈acyl, wherein the C₁-C₈acyl is optionally substituted with from 1 to 3 substituents independently selected from:
      (i) hydroxyl,
      (ii) CN,
      (iii) C₁-C₆alkoxy,
      (iv) C₁-C₆alkyl,
      (v) C₁-C₈acyl,
      (vi) NH₂,
      (vii) (C₁-C₆alkyl)amino,
      (viii) di(C₁-C₆alkyl)amino,
      (ix) CO₂H,
      (x) (C₁-C₆alkoxy)carbonyl,
      (xi) C₁-C₆perfluoroalkyl,
      (xii) and halogen;
   c) C₁-C₆alkyl optionally substituted with from 1 to 3 substituents independently selected from:
      (i) C₃-C₈cycloalkyl,
      (ii) C₁-C₆alkoxy,
      (iii) C₁-C₈acyl,
      (iv) CN,
      (v) (C₁-C₆alkoxy)carbonyl,
      (vi) CO₂H,
      (vii) hydroxyl,
      (viii) C₁-C₉heterocycle,
      (ix) and H₂NC(O)—;
   d) C₁-C₆perfluoroalkyl;
   e) C₂-C₆alkenyl;
   f) heteroaryl(C₁-C₆alkyl) wherein the ring portion of the heteroaryl(C₁-C₆alkyl) group is optionally substituted with from 1 to 3 substituents independently selected from:
      (i) C₁-C₆alkylC(O)NH—,
      (ii) C₁-C₆alkoxy,
      (iii) halogen,
      (iv) NH₂,
      (v) and C₁-C₆alkyl;
   g) (C₆-C₁₄aryl)alkyl, wherein the ring portion of the (C₆-C₁₄aryl)alkyl group is optionally substituted with 1 to 3 substituents independently selected from:
      (i) halogen,
      (ii) C₁-C₆alkyl,
      (iii) NH₂,
      (iv) (C₁-C₆alkyl)amino,
      (v) di(C₁-C₆alkyl)amino,
      (vi) hydroxyl,
      (vii) C₁-C₆alkoxy,
      (viii) C₁-C₈acyl,
      (ix) and C₁-C₉heteroaryl;
   h) HC(O)—;
   i) C₁-C₆perfluoroalkyl;
   j) —S(O)_{q}—(C₁-C₆alkyl);
   k) —S(O)_{q}-aryl;
   l) R¹⁹R²⁰NC(O);
   m) (C₁-C₉heteroaryl)-NH—C(S)—;
   n) (C₁-C₆alkyl)-NH—C(S)—;
   o) (C₁-C₆alkyl)-S—C(O)—;
   p) (C₆-C₁₄aryloxy)carbonyl;
   q) (C₂-C₆alkenyloxy)carbonyl;
   r) (C₂-C₆alkynyloxy)carbonyl;
   s) (C₁-C₆alkoxy)carbonyl optionally substituted with from 1 to 3 substituents independently selected from:
      (i) C₁-C₆alkoxy,
      (ii) halogen,
      (iii) C₆-C₁₄aryl,
      (iv) NH₂,
      (v) (C₁-C₆alkyl)amino-,
      (vi) di(C₁-C₆alkyl)amino-,
      (vii) and C₁-C₆alkyl;

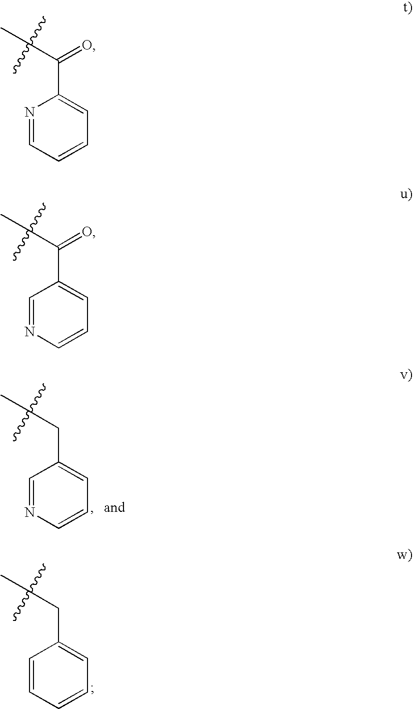

R₉ is —NHC(O)NR₁₀R₁₁, or —NHC(O)OR₁₂;
R₁₀ and R₁₁ are each independently —H, —OH, C₁-C₆alkoxy, C₆-C₁₄aryl, C₁-C₉heteroaryl, —C₃-C₈carbocycle, or —C₁-C₆alkyl; or R₁₀ and R₁₁ when taken together with the nitrogen to which they are attached to form a 3- to 7- membered nitrogen containing heterocycle wherein up to two of the carbon atoms of the heterocycle may be substituted with —N(R₁₅)—, —O—, or —S(O)ₙ;
R₁₂ is C₁-C₆alkyl, C₁-C₆hydroxylalkyl, or C₆-C₁₄aryl; and
R₁₅ is hydrogen, C₁-C₆alkyl, C₃-C₈carbocycle, C₆-C₁₄aryl, C₁-C₉heteroaryl, (C₁-C₆alkyl)amino, or substituted (C₆-C₁₄aryl)amino;
n is 0, 1, or 2;
q is 1 or 2;
R¹⁹ and R²⁰ are each independently:
a) H;
b) C₁-C₆alkyl optionally substituted with a substituent selected from:
   (i) C₁-C₆alkylC(O)NH—,
   (ii) NH₂,
   (iii) (C₁-C₆alkyl)amino, and
   (iv) di(C₁-C₆alkyl)amino;
c) C₃-C₈cycloalkyl;
d) C₆-C₁₄aryl optionally substituted with a substituent selected from:
   (i) halogen,
   (ii) and monocyclic C₁-C₆heterocycle wherein the monocyclic C₁-C₆heterocycle is optionally substituted with (C₁-C₆alkoxy)carbonyl;
e) C₁-C₉heteroaryl;
f) heteroaryl(C₁-C₆alkyl);
g) heterocyclyl(C₁-C₆alkyl);
h) (C₆-C₁₄aryl)alkyl, wherein the chain portion of the (C₆-C₁₄aryl)alkyl group is optionally substituted by a hydroxyl;
i) or monocyclic C₁-C₆heterocycle optionally substituted with a (C₁-C₆alkoxy)carbonyl;
   or R¹⁹ and R²⁰ when taken together with the nitrogen to which they are attached optionally form a 3- to 7- membered nitrogen-containing heterocycle wherein up to two of the carbon atoms of the heterocycle are optionally replaced with —N(H)—, —N(C₁-C₆alkyl)-, —N(C₆-C₁₄aryl)-, or —O—, and wherein the nitrogen-containing heterocycle is optionally substituted by a C₁-C₆alkyl; C₆-C₁₄aryl, (C₁-C₆alkoxy)C(O)NH—, or C₁-C₉heterocycle.

In another aspect, the invention provides methods of synthesizing compounds of the invention comprising:

a) reacting a compound of the formula IV:

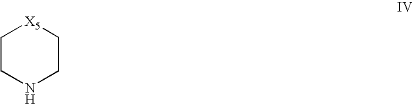

wherein X₅ is —O—, —S(O)ₙ,—, —CH₂—, —CH(OH)—, —C(O)—, —NH—, or the moiety

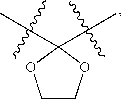

wherein n is 0, 1, or 2;

wherein any one or more of the methylene hydrogen atoms of formula IV can independently be substituted with C₁-C₃ alkyl, C₁-C₃ alkenyl, C₁-C₃ alkynyl, C₁-C₃ alkoxy, C₁-C₃ acyl, C₁-C₃ alkoxycarbonyl, amino(C₁-C₆alkyl), hydroxyl, ═O, fluorine or —CN;

with a compound of the formula V:

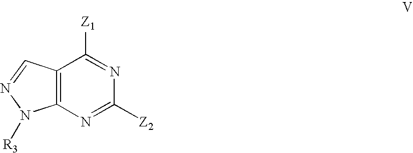

wherein Z₁ and Z₂ are each independently a halogen;

R₃ is hydrogen, optionally substituted C₁-C₆alkyl, optionally substituted C₂-C₁₀ alkenyl, optionally substituted C₂-C₁₀ alkynyl, optionally substituted acyl, optionally substituted C₆-C₁₄aryl, optionally substituted C₁-C₉heteroaryl, heterocyclyl(C₁-C₆alkyl), C₁-C₆hydroxylalkyl, alkylcarboxy, alkylamino-alkoxy, C₁-C₆perfluoroalkyl, —S(O)_{q}—(C₁-C₆alkyl) wherein the C₁-C₆alkyl of —S(O)_{q}—(C₁-C₆alkyl) can be optionally substituted, —S(O)_{q}-aryl wherein the C₆-C₁₄aryl of —S(O)_{q}-aryl can be optionally substituted, optionally substituted C₃-C₈carbocycle, optionally substituted 6- to 10-membered bicyclic carbocycle, 4- to 7-membered monocyclic C₁-C₆heterocycle, nitrogen containing 4- to 7-membered monocyclic C₁-C₆heterocycle, 6- to 10-membered bicyclic heterocycle, or nitrogen-containing 6- to 10-membered bicyclic heterocycle;

under conditions effective to substitute Z₁ with the compound of formula V thereby providing a compound having the formula VI:

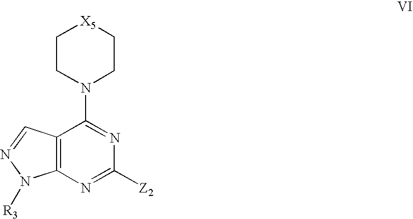

wherein any one or more of the methylene hydrogen atoms of the morpholinyl moiety can independently be substituted with C₁-C₃ alkyl, C₁-C₃ alkenyl, C₁-C₃ alkynyl, C₁-C₃ alkoxy, C₁-C₃ acyl, C₁-C₃ alkoxycarbonyl, amino(C₁-C₆alkyl), hydroxyl, ═O, fluorine, or —CN;

X₅ is —O, —S(O)ₙ,—, —CH₂—, —CH(OH)—, —C(O)—, —NH—, or the moiety

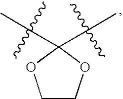

wherein n is 0, 1, or 2; and

Z₂ is a halogen; and

R₃ is hydrogen, optionally substituted C₁-C₆alkyl, optionally substituted C₂-C₁₀ alkenyl, optionally substituted C₂-C₁₀ alkynyl, optionally substituted acyl, optionally substituted C₆-C₁₄aryl, optionally substituted C₁-C₉heteroaryl, heterocyclyl(C₁-C₆alkyl), C₁-C₆hydroxylalkyl, C₁-C₆alkylcarboxy, alkylamino-alkoxy, C₁-C₆perfluoroalkyl, —S(O)_{q}—(C₁-C₆alkyl) wherein the C₁-C₆alkyl of —S(O)_{q}—(C₁-C₆alkyl) can be optionally substituted, —S(O)_{q}-aryl wherein the C₆-C₁₄aryl of —S(O)_{q}-aryl can be optionally substituted, optionally substituted C₃-C₈carbocycle, optionally substituted 6- to 10-membered bicyclic carbocycle, 4- to 7-membered monocyclic C₁-C₆heterocycle, nitrogen containing 4- to 7-membered monocyclic C₁-C₆heterocycle, 6- to 10-membered bicyclic heterocycle, or nitrogen-containing 6- to 10-membered bicyclic heterocycle;

b) reacting the compound of Formula VI with a boronic acid of the structure:

R₂B(OH)₂

wherein R₂ is optionally substituted C₆-C₁₄aryl, optionally substituted C₁-C₉heteroaryl, optionally substituted C₆-C₁₄arylurea, optionally substituted C₆-C₁₄arylcarbamate, optionally substituted —HC═CH-aryl, or optionally substituted —HC═CH-heteroaryl;

under conditions effective to substitute the Z₂ of formula VI with R₂ thereby providing a compound of formula VII:

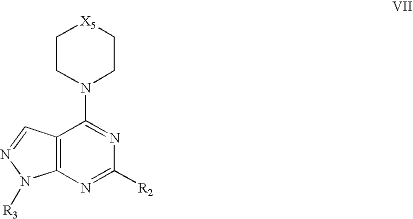

wherein any one or more of the methylene hydrogen atoms of the morpholinyl moiety can independently be substituted with C₁-C₃ alkyl, C₁-C₃ alkenyl, C₁-C₃ alkynyl, C₁-C₃ alkoxy, C₁-C₃ acyl, C₁-C₃ alkoxycarbonyl, amino(C₁-C₆alkyl), hydroxyl, ═O, fluorine, or —CN;

X₅ is —S(O)ₙ,—, —CH₂—, —CH(OH)—, —C(O)—, —NH—, or the moiety

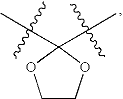

wherein n is 0, 1, or 2;

R₂ is optionally substituted C₆-C₁₄aryl, optionally substituted C₁-C₉heteroaryl, optionally substituted C₆-C₁₄arylurea, optionally substituted C₆-C₁₄arylcarbamate, optionally substituted —HC═CH-aryl, or optionally substituted —HC═CH-heteroaryl;

R₃ is hydrogen, optionally substituted C₁-C₆alkyl, optionally substituted C₂-C₁₀ alkenyl, optionally substituted C₂-C₁₀ alkynyl, optionally substituted acyl, optionally substituted C₆-C₁₄aryl, optionally substituted C₁-C₉heteroaryl, heterocyclyl(C₁-C₆alkyl), C₁-C₆hydroxylalkyl, C₁-C₆alkylcarboxy, alkylamino-alkoxy, C₁-C₆perfluoroalkyl, —S(O)_{q}—(C₁-C₆alkyl) wherein the C₁-C₆alkyl of —S(O)_{q}—(C₁-C₆alkyl) can be optionally substituted, —S(O)_{q}-aryl wherein the C₆-C₁₄aryl of —S(O)_{q}-aryl can be optionally substituted, optionally substituted C₃-C₈carbocycle, optionally substituted 6- to 10-membered bicyclic carbocycle, 4- to 7-membered monocyclic C₁-C₆heterocycle, nitrogen containing 4- to 7-membered monocyclic C₁-C₆heterocycle, 6- to 10-membered bicyclic heterocycle, or nitrogen-containing 6- to 10-membered bicyclic heterocycle; and q is 0, 1 or 2.

In another aspect, the invention provides methods of synthesizing compounds of Formula Ia comprising:
a) reacting a compound of the formula (IV):

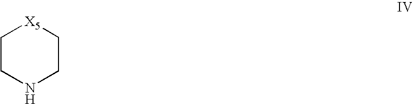

wherein X₅ is —O— or —S(O)ₙ,— wherein any one or more of the ring carbons of the compound of formula (IV) can independently be substituted with C₁-C₃alkyl, C₁-C₃alkenyl, C₁-C₃alkynyl, C₁-C₃alkoxy, C₁-C₃acyl, C₁-C₃alkoxycarbonyl, amino(C₁-C₆alkyl), hydroxyl, fluorine, or —CN, where any two hydrogen atoms attached to the same carbon atom can be, taken together with the carbon to which they are attached, can be replaced by an oxygen atom, the oxygen atom taken together with the carbon to which it is attached, forming a carbonyl (C═O),
and wherein n is an integer from 0 to 2;
with a compound of the formula V:

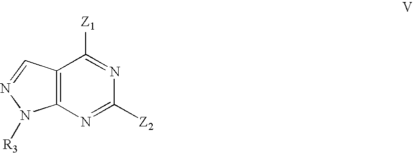

wherein Z₁ and Z₂ are each independently a halogen;
   R₃ is as defined in claim 1;
thereby providing a compound having the formula VI:

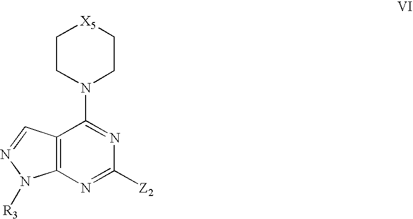

wherein any one or more of the ring hydrogen atoms of the group

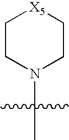

of Formula VI can independently be replaced with C₁-C₃alkyl, C₁-C₃alkenyl, C₁-C₃alkynyl, C₁-C₃alkoxy, C₁-C₃acyl, C₁-C₃alkoxycarbonyl, amino(C₁-C₆alkyl), hydroxyl, fluorine, or —CN, where any two hydrogen atoms attached to the same carbon atom can be, taken together with the carbon to which they are attached, can be replaced by an oxygen atom, the oxygen atom taken together with the carbon to which it is attached, forming a carbonyl (C═O),
b) reacting the compound of formula VI with a boronic acid of the structure:

R₂B(OH)₂
wherein R₂ is as defined in claim 1, thereby providing a compound of the formula VII:

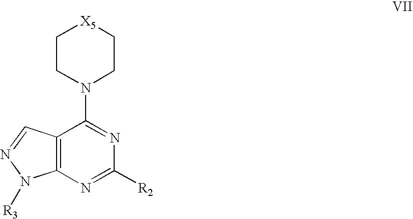

wherein any one or more of the ring hydrogen atoms of the group

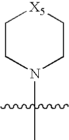

in Formula VII can independently be replaced with C₁-C₃alkyl, C₁-C₃alkenyl, C₁-C₃alkynyl, C₁-C₃alkoxy, C₁-C₃acyl, C₁-C₃alkoxycarbonyl, amino(C₁-C₆alkyl), hydroxyl, fluorine, or —CN, where any two hydrogen atoms attached to the same carbon atom can be, taken together with the carbon to which they are attached, can be replaced by an oxygen atom to form a carbonyl (C═O).

In other aspects, the invention provides pharmaceutical compositions comprising compounds or pharmaceutically acceptable salts of compounds of Formula (I), Formula (Ia), Formula (II), Formula (III), Formula (IIIa), Formula (IIIb), and Formula (IIIc) and a pharmaceutically acceptable carrier.

In one aspect, the compounds or pharmaceutically acceptable salts of the compounds of Formula (I), Formula (Ia), Formula (II), Formula (III), Formula (IIIa), Formula (IIIb), and Formula (IIIc) are useful as mTOR inhibitors.

In one aspect, the compounds or pharmaceutically acceptable salts of the compounds of Formula (I), Formula (Ia), Formula (II), Formula (III), Formula (IIIa), Formula (IIIb), and Formula (IIIc) are useful as PI3K inhibitors.

In one embodiment, the invention provides methods for treating an mTOR-related disorder, comprising administering to a mammal in need thereof, the compounds or pharmaceutically acceptable salts of compounds of Formula (I), Formula (Ia), Formula (II), Formula (III), Formula (IIIa), Formula (IIIb), and Formula (IIIc) in an amount effective to treat a mTOR-related disorder.

In one embodiment, the invention provides methods for treating a PI3K-related disorder, comprising administering to a mammal in need thereof the compounds or pharmaceutically acceptable salts of compounds of Formula (I), Formula (Ia), Formula (II), Formula (III), Formula (IIIa), Formula (IIIb), and Formula (IIIc) in an amount effective to treat a PI3K-related disorder.

In other aspects, the invention provides further methods of synthesizing the compounds or pharmaceutically acceptable salts of compounds of Formula (I), Formula (Ia), Formula (II), Formula (III), Formula (IIIa), Formula (IIIb), and Formula (IIIc).

### DETAILED DESCRIPTION OF THE INVENTION

### The Pyrazolopyrimidine Analogs of Formula (I)

The present invention provides Pyrazolopyrimidine Analogs according to Formula (I), below:

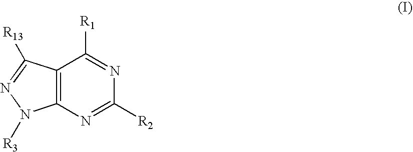

and pharmaceutically acceptable salts, hydrates, and solvates thereof, wherein:

R₁, R₂, R₃, and R₁₃ are as defined above for the compounds of Formula (I).

In one embodiment, X₅ is —O—.

In another embodiment, R₁ is unsubstituted N-morpholinyl.

In one embodiment, R₂ is optionally substituted C₆-C₁₄aryl.

In one embodiment, R₂ is optionally substituted C₁-C₉heteroaryl.

In one embodiment, R₂ is optionally substituted C₁-C₆alkyl.

In one embodiment, R₂ is optionally substituted C₂-C₁₀alkenyl.

In one embodiment, R₂ is optionally substituted C₆-C₁₄arylcarbamate.

In one embodiment, R₂ is optionally substituted C₆-C₁₄arylurea.

In one embodiment, R₂ is optionally substituted —HC═CH-aryl.

In one embodiment, R₂ is optionally substituted —HC═CH-heteroaryl.

In another embodiment, R₃ is hydrogen.

In another embodiment, R₃ is optionally substituted C₁-C₆alkyl.

In another embodiment, R₃ is optionally substituted C₂-C₁₀alkenyl.

In another embodiment, R₃ is optionally substituted C₂-C₁₀alkynyl.

In another embodiment, R₃ is optionally substituted C₆-C₁₄aryl.

In another embodiment, R₃ is optionally substituted C₁-C₉heteroaryl.

In another embodiment, R₃ is heterocyclyl(C₁-C₆alkyl)

In another embodiment, R₃ is C₁-C₆hydroxylalkyl.

In another embodiment, R₃ is alkylcarboxy.

In another embodiment, R₃ is alkylamino-alkoxy.

In another embodiment, R₃ is C₁-C₆perfluoroalkyl.

In another embodiment, R₃ is —S(O)_{q}—(C₁-C₆alkyl) wherein the C₁-C₆alkyl of —S(O)_{q}—(C₁-C₆alkyl) can be optionally substituted.

In another embodiment, R₃ is —S(O)_{q}-aryl wherein the C₆-C₁₄aryl of —S(O)_{q}-aryl can be optionally substituted.

In another embodiment, R₃ is optionally substituted C₃-C₈carbocycle.

In another embodiment, R₃ is a 4- to 7-membered monocyclic C₁-C₆heterocycle.

In another embodiment, R₃ is a nitrogen containing 4- to 7-membered monocyclic C₁-C₆heterocycle.

In another embodiment, R₃ is a 6- to 10-membered bicyclic heterocycle.

In one embodiment, R₁₃ is hydrogen.

In one embodiment, R₁₃ is halogen.

In one embodiment, R₁₃ is optionally substituted C₁-C₆alkyl.

In one embodiment, R₁₃ is optionally substituted C₆-C₁₄aryl.

In one embodiment, R₁₃ is optionally substituted C₁-C₉heteroaryl.

In another embodiment, q is 1 or 2.

In another embodiment, q is 1.

The invention also relates to compounds of Formula II:

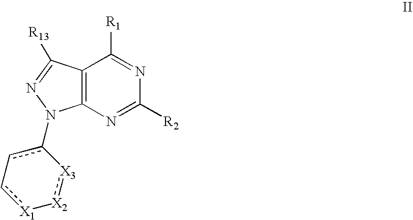

and pharmaceutically acceptable salts, hydrates, and solvates thereof, wherein

R₁, R₂, R₁₃, X₁, X₂, and X₃ are as defined above for the compounds of Formula II.

In one embodiment, X₅, is —O—.

In another embodiment, R₁ is unsubstituted N-morpholinyl.

In one embodiment, R₂ is optionally substituted C₆-C₁₄aryl.

In one embodiment, R₂ is optionally substituted C₁-C₉heteroaryl.

In one embodiment, R₂ is optionally substituted C₁-C₆alkyl.

In one embodiment, R₂ is optionally substituted C₂-C₁₀alkenyl.

In one embodiment, R₂ is optionally substituted C₆-C₁₄arylcarbamate.

In one embodiment, R₂ is optionally substituted C₆-C₁₄arylurea.

In one embodiment, R₂ is optionally substituted —HC═CH-aryl.

In one embodiment, R₂ is optionally substituted —HC═CH-heteroaryl.

In one embodiment, R₁₃ is hydrogen.

In one embodiment, R₁₃ is halogen.

In one embodiment, R₁₃ is optionally substituted C₁-C₆alkyl.

In one embodiment, R₁₃ is optionally substituted C₆-C₁₄aryl.

In one embodiment, R₁₃ is optionally substituted C₁-C₉heteroaryl.

In one embodiment, X₁ is —N(R₄)—.

In one embodiment, X₁ is —CH(OH)—

In one embodiment, X₁ is —C(O)—.

In one embodiment, X₁ is —O—.

In one embodiment, X₁ is —CH—.

In one embodiment, X₁ is —CH₂—.

In one embodiment, X₁ is

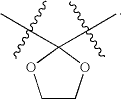

In embodiment, X₂ is —N(H)—.

In embodiment, X₂ is —NBOC—.

In embodiment, X₃ is —O—.

In embodiment, X₃ is optionally substituted —CH₂—.

In one embodiment, X₁ and X₂ are each —CH₂— and X₃ is —O—.

In one embodiment, X₂ is —CH₂—.

In one embodiment, R₄ is —H.

In one embodiment, R₄ is optionally substituted C₁-C₆alkyl.

In one embodiment, R₄ is —C(O)alkyl.

In one embodiment, R₄ is —C(O)alkoxy.

In one embodiment, R₄ is —C(O)NR₅R₆.

In one embodiment, R₄ is

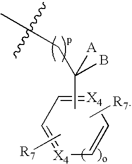

In one embodiment, p is 0.

In one embodiment, p is 1.

In one embodiment, A is hydrogen.

In another embodiment, A and B are both hydrogen.

In another embodiment, A and B together form a carbonyl.

In another embodiment, one X₄ is —CH—.

In another embodiment, one X₄ is —N—.

In another embodiment, one X₄ is —O—.

In another embodiment, one X₄ is —N⁺(O⁻)—.

In another embodiment, o is 1.

In another embodiment, o is 0.

In one embodiment R₅ and R₆ are each independently —H, optionally substituted alkyl, optionally substituted C₆-C₁₄aryl, or optionally substituted C₁-C₉heteroaryl.

In another embodiment R₅ and R₆ are taken together with the nitrogen to which there are attached to form a 5 to 7 membered nitrogen containing heterocycle.

In one embodiment, R₇ is —H.

In one embodiment, R₇ is —OH.

In one embodiment, R₇ is halogen.

In one embodiment, R₇ is optionally substituted alkyl.

In one embodiment, R₇ is optionally substituted alkoxy.

In one embodiment, R₇ is optionally substituted acyl.

In one embodiment, R₇ is optionally substituted amine.

In one embodiment, R₇ is optionally substituted amide.

In one embodiment, R₇ is —CN.

In one embodiment, there is one R₇.

In one embodiment, there are more than one R₇.

In one embodiment, R₈ is optionally substituted C₁-C₆alkyl.

In one embodiment, R₈ is optionally substituted —C(O)—C₁-C₆alkyl.

In one embodiment, R₈ is —C(O)NR₅R₆.

In one embodiment, R₈ is —C(O)OC₁-C₆alkyl.

In one embodiment, the structure

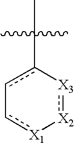

contains no double bond.

In another embodiment, the structure

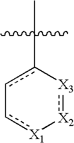

contains three double bonds.

Illustrative compounds of Formula II are exemplified by the following compounds:

| Example | Compound Name |
|---|---|
| 24 | 3-{4-morpholin-4-yl-1-[1-(pyridin-3-ylcarbonyl)piperidin-4-yl]-1H-pyrazolo[3,4- |
| | d]pyrimidin-6-yl}phenol |
| 25 | 3-[1-(1-benzoylpiperidin-4-yl)-4-morpholin-4-yl-1H-pyrazolo[3,4-d]pyrimidin-6- |
| | yl]phenol |
| 28 | 3-{1-[1-(4-methoxybenzoyl)piperidin-4-yl]-4-morpholin-4-yl-1H-pyrazolo[3,4- |
| | d]pyrimidin-6-yl}phenol |
| 37 | 3-[1-(1-benzylpiperidin-4-yl)-4-morpholin-4-yl-1H-pyrazolo[3,4-d]pyrimidin-6- |
| | yl]benzoic acid |
| 38 | 6-(1H-indol-5-yl)-4-morpholin-4-yl-1-[1-(pyridin-3-ylcarbonyl)piperidin-4-yl]-1H- |
| | pyrazolo[3,4-d]pyrimidine |
| 39 | 6-(1H-indol-5-yl)-4-morpholin-4-yl-1-[1-(pyridin-3-ylmethyl)piperidin-4-yl]-1H- |
| | pyrazolo[3,4-d]pyrimidine |
| 47 | 6-(1H-indol-5-yl)-4-morpholin-4-yl-1-[1-(pyridin-3-ylmethyl)piperidin-4-yl]-1H- |
| | pyrazolo[3,4-d]pyrimidine |
| 80 | N-{4-[1-(1-benzylpiperidin-4-yl)-4-morpholin-4-yl-1H-pyrazolo[3,4-d]pyrimidin-6- |
| | yl]phenyl}acetamide |
| 90 | 1-cyclohexyl-6-(1H-indol-5-yl)-4-morpholin-4-yl-1H-pyrazolo[3,4-d]pyrimidine |
| 91 | 3-(1-cyclohexyl-4-morpholin-4-yl-1H-pyrazolo[3,4-d]pyrimidin-6-yl)phenol |
| 101 | methyl [4-(4-morpholin-4-yl-1-phenyl-1H-pyrazolo[3,4-d]pyrimidin-6- |
| | yl)phenyl]carbamate |
| 129 | 5-(4-morpholin-4-yl-1-phenyl-1H-pyrazolo[3,4-d]pyrimidin-6-yl)pyridin-2-amine |
| 154 | methyl {4-[1-(1-benzylpiperidin-4-yl)-4-morpholin-4-yl-1H-pyrazolo[3,4- |
| | d]pyrimidin-6-yl]phenyl}carbamate |
| 158 | N-(4-{4-morpholin-4-yl-1-[1-(pyridin-3-ylmethyl)piperidin-4-yl]-1H-pyrazolo[3,4- |
| | d]pyrimidin-6-yl}phenyl)acetamide |
| 159 | N-(4-{4-morpholin-4-yl-1-[1-(pyridin-3-ylcarbonyl)piperidin-4-yl]-1H- |
| | pyrazolo[3,4-d]pyrimidin-6-yl}phenyl)acetamide |
| 172 | 1-{1-[(6-fluoropyridin-3-yl)methyl]piperidin-4-yl}-6-(1H-indol-5-yl)-4-morpholin- |
| | 4-yl-1H-pyrazolo[3,4-d]pyrimidine |
| 173 | 1-{1-[(6-bromopyridin-3-yl)methyl]piperidin-4-yl}-6-(1H-indol-5-yl)-4-morpholin- |
| | 4-yl-1H-pyrazolo[3,4-d]pyrimidine |
| 196 | 1-{1-[(6-chloropyridin-3-yl)carbonyl]piperidin-4-yl}-6-(1H-indol-5-yl)-4- |
| | morpholin-4-yl-1H-pyrazolo[3,4-d]pyrimidine |
| 198 | 2-({4-[6-(1H-indol-5-yl)-4-morpholin-4-yl-1H-pyrazolo[3,4-d]pyrimidin-1- |
| | yl]piperidin-1-yl}carbonyl)pyridin-3-ol |
| 199 | 6-(1H-indol-5-yl)-1-{1-[(4-methylpyridin-3-yl)carbonyl]piperidin-4-yl}-4- |
| | morpholin-4-yl-1H-pyrazolo[3,4-d]pyrimidine |
| 200 | 6-(1H-indol-5-yl)-1-{1-[(2-methylpyridin-3-yl)carbonyl]piperidin-4-yl}-4- |
| | morpholin-4-yl-1H-pyrazolo[3,4-d]pyrimidine |
| 202 | 6-(1H-indol-5-yl)-1-{1-[(6-methylpyridin-3-yl)carbonyl]piperidin-4-yl}-4- |
| | morpholin-4-yl-1H-pyrazolo[3,4-d]pyrimidine |
| 210 | 5-{4-morpholin-4-yl-1-[1-(pyridin-3-ylmethyl)piperidin-4-yl]-1H-pyrazolo[3,4- |
| | d]pyrimidin-6-yl}pyridin-2-amine |
| 212 | 1-Butyl-3-(4-{4-morpholin-4-yl-1-[1-(pyridin-3-ylmethyl)piperidin-4-yl]-1H- |
| | pyrazolo[3,4-d]pyrimidin-6-yl}phenyl)urea |
| 213 | methyl (4-{4-morpholin-4-yl-1-[1-(pyridin-3-ylcarbonyl)piperidin-4-yl]-1H- |
| | pyrazolo[3,4-d]pyrimidin-6-yl}phenyl)carbamate |
| 214 | 5-{4-morpholin-4-yl-1-[1-(pyridin-3-ylcarbonyl)piperidin-4-yl]-1H-pyrazolo[3,4- |
| | d]pyrimidin-6-yl}pyridin-2-amine |
| 215 | 5-{4-morpholin-4-yl-1-[1-(pyridin-3-ylcarbonyl)piperidin-4-yl]-1H-pyrazolo[3,4- |
| | d]pyrimidin-6-yl}pyrimidin-2-amine |
| 220 | N-(4-{4-morpholin-4-yl-1-[1-(pyridin-3-ylmethyl)piperidin-4-yl]-1H-pyrazolo[3,4- |
| | d]pyrimidin-6-yl}phenyl)acetamide |
| 221 | N-(4-{4-morpholin-4-yl-1-[1-(pyridin-3-ylcarbonyl)piperidin-4-yl]-1H- |
| | pyrazolo[3,4-d]pyrimidin-6-yl}phenyl)acetamide |
| 235 | 1-methyl-3-{4-[4-morpholin-4-yl-1-(tetrahydro-2H-pyran-2-yl)-1H-pyrazolo[3,4- |
| | d]pyrimidin-6-yl]phenyl}urea |
| 236 | 1-methyl-3-[4-(4-morpholin-4-yl-1H-pyrazolo[3,4-d]pyrimidin-6-yl)phenyl]urea |
| 244 | methyl 4-[6-(1H-indol-5-yl)-4-morpholin-4-yl-1H-pyrazolo[3,4-d]pyrimidin-1- |
| | yl]piperidine-1-carboxylate |
| 252 | 1-{4-[1-(1-benzylpiperidin-4-yl)-4-morpholin-4-yl-1H-pyrazolo[3,4-d]pyrimidin-6- |
| | yl]phenyl}urea |
| 254 | 1-{4-[1-(1-benzylpiperidin-4-yl)-4-morpholin-4-yl-1H-pyrazolo[3,4-d]pyrimidin-6- |
| | yl]phenyl}-3-propylurea |
| 256 | 1-{4-[1-(1-benzylpiperidin-4-yl)-4-morpholin-4-yl-1H-pyrazolo[3,4-d]pyrimidin-6- |
| | yl]phenyl}-3-isopropylurea |
| 257 | 1-{4-[1-(1-benzylpiperidin-4-yl)-4-morpholin-4-yl-1H-pyrazolo[3,4-d]pyrimidin-6- |
| | yl]phenyl}-3-phenylurea |
| 263 | 1-allyl-3-{4-[1-(1-benzylpiperidin-4-yl)-4-morpholin-4-yl-1H-pyrazolo[3,4- |
| | d]pyrimidin-6-yl]phenyl}urea |
| 267 | 1-(2-aminoethyl)-3-{4-[1-(1-benzylpiperidin-4-yl)-4-morpholin-4-yl-1H- |
| | pyrazolo[3,4-d]pyrimidin-6-yl]phenyl}urea |
| 269 | 1-{4-[1-(1-benzylpiperidin-4-yl)-4-morpholin-4-yl-1H-pyrazolo[3,4-d]pyrimidin-6- |
| | yl]phenyl}-3-(3-hydroxypropyl)urea |
| 285 | 4-(4-morpholin-4-yl-1H-pyrazolo[3,4-d]pyrimidin-6-yl)aniline |
| 287 | 1-{4-[1-(1-benzylpiperidin-4-yl)-4-morpholin-4-yl-1H-pyrazolo[3,4-d]pyrimidin-6- |
| | yl]phenyl}-3-ethoxyurea |
| 288 | 1-{4-[1-(1-benzylpiperidin-4-yl)-4-morpholin-4-yl-1H-pyrazolo[3,4-d]pyrimidin-6- |
| | yl]phenyl}-3-(2-fluoroethyl)urea |
| 289 | 1-{4-[1-(1-benzylpiperidin-4-yl)-4-morpholin-4-yl-1H-pyrazolo[3,4-d]pyrimidin-6- |
| | yl]phenyl}-3-(2,2,2-trifluoroethyl)urea |
| 292 | N-[4-(4-morpholin-4-yl-1-phenyl-1H-pyrazolo[3,4-d]pyrimidin-6- |
| | yl)phenyl]hydrazinecarboxamide |
| 293 | 1-hydroxy-3-[4-(4-morpholin-4-yl-1-phenyl-1H-pyrazolo[3,4-d]pyrimidin-6- |
| | yl)phenyl]urea |
| 297 | 1-methyl-3-[4-(4-morpholin-4-yl-1-phenyl-1H-pyrazolo[3,4-d]pyrimidin-6- |
| | yl)phenyl]urea |
| 300 | 1-{4-[1-(1-benzylpiperidin-4-yl)-4-morpholin-4-yl-1H-pyrazolo[3,4-d]pyrimidin-6- |
| | yl]phenyl}-3-[2-(methylamino)ethyl]urea |
| 305 | 1-hydroxy-3-(4-{4-morpholin-4-yl-1-[1-(pyridin-3-ylcarbonyl)piperidin-4-yl]-1H- |
| | pyrazolo[3,4-d]pyrimidin-6-yl}phenyl)urea |
| 306 | N-(4-{4-morpholin-4-yl-1-[1-(pyridin-3-ylcarbonyl)piperidin-4-yl]-1H- |
| | pyrazolo[3,4-d]pyrimidin-6-yl}phenyl)hydrazinecarboxamide |
| 308 | 1-methoxy-3-(4-{4-morpholin-4-yl-1-[1-(pyridin-3-ylcarbonyl)piperidin-4-yl]-1H- |
| | pyrazolo[3,4-d]pyrimidin-6-yl}phenyl)urea |
| 309 | N-{4-[1-(1-benzylpiperidin-4-yl)-4-morpholin-4-yl-1H-pyrazolo[3,4-d]pyrimidin-6- |
| | yl]phenyl}hydrazinecarboxamide |
| 311 | 1-{4-[1-(1-benzylpiperidin-4-yl)-4-morpholin-4-yl-1H-pyrazolo[3,4-d]pyrimidin-6- |
| | yl]phenyl}-3-cyclopropylurea |
| 313 | 1-(4-{4-morpholin-4-yl-1-[1-(pyridin-3-ylmethyl)piperidin-4-yl]-1H-pyrazolo[3,4- |
| | d]pyrimidin-6-yl}phenyl)urea |
| 325 | 1-{4-[1-(1-benzylpiperidin-4-yl)-4-morpholin-4-yl-1H-pyrazolo[3,4-d]pyrimidin-6- |
| | yl]phenyl}-3-methylthiourea |
| 334 | 1-methyl-3-[4-(1-{1-[(4-methylpiperazin-1-yl)carbonyl]piperidin-4-yl}-4- |
| | morpholin-4-yl-1H-pyrazolo[3,4-d]pyrimidin-6-yl)phenyl]urea |
| 335 | 4-(6-{4-[(methylcarbamoyl)amino]phenyl}-4-morpholin-4-yl-1H-pyrazolo[3,4- |
| | d]pyrimidin-1-yl)-N-pyridin-3-ylpiperidine-1-carboxamide |
| 342 | 1-[2-(methylamino)ethyl]-3-(4-{4-morpholin-4-yl-1-[1-(pyridin-3- |
| | ylmethyl)piperidin-4-yl]-1H-pyrazolo[3,4-d]pyrimidin-6-yl}phenyl)urea |
| 346 | 1-[2-(methylamino)ethyl]-3-(4-{4-morpholin-4-yl-1-[1-(pyridin-3- |
| | ylcarbonyl)piperidin-4-yl]-1H-pyrazolo[3,4-d]pyrimidin-6-yl}phenyl)urea |
| 348 | 1-{4-[1-(1-benzylpiperidin-4-yl)-4-morpholin-4-yl-1H-pyrazolo[3,4-d]pyrimidin-6- |
| | yl]-2-fluorophenyl}-3-methylurea |
| 349 | 1-{4-[1-(1-benzylpiperidin-4-yl)-4-morpholin-4-yl-1H-pyrazolo[3,4-d]pyrimidin-6- |
| | yl]-2-fluorophenyl}-3-ethylurea |
| 368 | 1-(4-{1-[1-(4-cyanobenzoyl)piperidin-4-yl]-4-morpholin-4-yl-1H-pyrazolo[3,4- |
| | d]pyrimidin-6-yl}phenyl)-3-methylurea |
| 372 | 1-ethyl-3-(2-fluoro-4-{4-morpholin-4-yl-1-[1-(pyridin-3-ylmethyl)piperidin-4-yl]- |
| | 1H-pyrazolo[3,4-d]pyrimidin-6-yl}phenyl)urea |
| 374 | 1-(2-fluoroethyl)-3-(2-fluoro-4-{4-morpholin-4-yl-1-[1-(pyridin-3- |
| | ylmethyl)piperidin-4-yl]-1H-pyrazolo[3,4-d]pyrimidin-6-yl}phenyl)urea |
| 377 | 1-methyl-3-(4-{4-morpholin-4-yl-1-[1-(pyridin-3-ylmethyl)piperidin-4-yl]-1H- |
| | pyrazolo[3,4-d]pyrimidin-6-yl}phenyl)thiourea |
| 383 | Ethyl {4-[1-(1-benzoylpiperidin-4-yl)-4-morpholin-4-yl-1H-pyrazolo[3,4- |
| | d]pyrimidin-6-yl]phenyl}carbamate |
| 397 | 1-(4-{1-[1-(3-hydroxybenzyl)piperidin-4-yl]-4-morpholin-4-yl-1H-pyrazolo[3,4- |
| | d]pyrimidin-6-yl}phenyl)-3-methylurea |
| 398 | 1-(4-{1-[1-(3-hydroxy-4-methoxybenzyl)piperidin-4-yl]-4-morpholin-4-yl-1H- |
| | pyrazolo[3,4-d]pyrimidin-6-yl}phenyl)-3-methylurea |
| 402 | 1-(4-{1-[1-(2-furylmethyl)piperidin-4-yl]-4-morpholin-4-yl-1H-pyrazolo[3,4- |
| | d]pyrimidin-6-yl}phenyl)-3-methylurea |
| 406 | 2-cyano-1-(4-{4-morpholin-4-yl-1-[1-(pyridin-3-ylmethyl)piperidin-4-yl]-1H- |
| | pyrazolo[3,4-d]pyrimidin-6-yl}phenyl)guanidine |
| 407 | 2-cyano-1-methyl-3-(4-{4-morpholin-4-yl-1-[1-(pyridin-3-ylmethyl)piperidin-4-yl]- |
| | 1H-pyrazolo[3,4-d]pyrimidin-6-yl}phenyl)guanidine |
| 408 | 2-cyano-1-ethyl-3-(4-{4-morpholin-4-yl-1-[1-(pyridin-3-ylmethyl)piperidin-4-yl]- |
| | 1H-pyrazolo[3,4-d]pyrimidin-6-yl}phenyl)guanidine |
| 409 | N′-cyano-N-(4-{4-morpholin-4-yl-1-[1-(pyridin-3-ylmethyl)piperidin-4-yl]-1H- |
| | pyrazolo[3,4-d]pyrimidin-6-yl}phenyl)carbamimidic acid |
| 410 | methyl N′-cyano-N-(4-{4-morpholin-4-yl-1-[1-(pyridin-3-ylmethyl)piperidin-4-yl]- |
| | 1H-pyrazolo[3,4-d]pyrimidin-6-yl}phenyl)imidocarbamate |
| 411 | 2-cyano-1-(4-{4-morpholin-4-yl-1-[1-(pyridin-3-ylcarbonyl)piperidin-4-yl]-1H- |
| | pyrazolo[3,4-d]pyrimidin-6-yl}phenyl)guanidine |
| 412 | 2-cyano-1-methyl-3-(4-{4-morpholin-4-yl-1-[1-(pyridin-3-ylcarbonyl)piperidin-4- |
| | yl]-1H-pyrazolo[3,4-d]pyrimidin-6-yl}phenyl)guanidine |
| 429 | 1-methyl-3-(4-{1-[1-(2-methylbenzyl)piperidin-4-yl]-4-morpholin-4-yl-1H- |
| | pyrazolo[3,4-d]pyrimidin-6-yl}phenyl)urea |
| 430 | 1-methyl-3-(4-{1-[1-(3-methylbenzyl)piperidin-4-yl]-4-morpholin-4-yl-1H- |
| | pyrazolo[3,4-d]pyrimidin-6-yl}phenyl)urea |
| 431 | 1-methyl-3-(4-{1-[1-(4-methylbenzyl)piperidin-4-yl]-4-morpholin-4-yl-1H- |
| | pyrazolo[3,4-d]pyrimidin-6-yl}phenyl)urea |
| 432 | 6-(1H-indazol-5-yl)-4-morpholin-4-yl-1-phenyl-1H-pyrazolo[3,4-d]pyrimidine |
| 440 | methyl [4-(1-{1-[(4-methylpyridin-3-yl)carbonyl]piperidin-4-yl}-4-morpholin-4-yl- |
| | 1H-pyrazolo[3,4-d]pyrimidin-6-yl)phenyl]carbamate |
| 441 | methyl (4-{4-morpholin-4-yl-1-[1-(pyridin-2-ylcarbonyl)piperidin-4-yl]-1H- |
| | pyrazolo[3,4-d]pyrimidin-6-yl}phenyl)carbamate |
| 442 | methyl {4-[1-(1-benzoylpiperidin-4-yl)-4-morpholin-4-yl-1H-pyrazolo[3,4- |
| | d]pyrimidin-6-yl]phenyl}carbamate |
| 443 | methyl (4-{1-[1-(2-fluorobenzoyl)piperidin-4-yl]-4-morpholin-4-yl-1H- |
| | pyrazolo[3,4-d]pyrimidin-6-yl}phenyl)carbamate |
| 444 | methyl (4-{1-[1-(2-chlorobenzoyl)piperidin-4-yl]-4-morpholin-4-yl-1H- |
| | pyrazolo[3,4-d]pyrimidin-6-yl}phenyl)carbamate |
| 445 | methyl (4-{1-[1-(4-fluorobenzoyl)piperidin-4-yl]-4-morpholin-4-yl-1H- |
| | pyrazolo[3,4-d]pyrimidin-6-yl}phenyl)carbamate |
| 446 | methyl (4-{1-[1-(2-methoxybenzoyl)piperidin-4-yl]-4-morpholin-4-yl-1H- |
| | pyrazolo[3,4-d]pyrimidin-6-yl}phenyl)carbamate |
| 447 | methyl (4-{1-[1-(4-cyanobenzoyl)piperidin-4-yl]-4-morpholin-4-yl-1H- |
| | pyrazolo[3,4-d]pyrimidin-6-yl}phenyl)carbamate |
| 449 | methyl (4-{1-[1-(2,4-difluorobenzoyl)piperidin-4-yl]-4-morpholin-4-yl-1H- |
| | pyrazolo[3,4-d]pyrimidin-6-yl}phenyl)carbamate |
| 454 | methyl [4-(1-{1-[(6-chloropyridin-3-yl)methyl]piperidin-4-yl}-4-morpholin-4-yl- |
| | 1H-pyrazolo[3,4-d]pyrimidin-6-yl)phenyl]carbamate |
| 461 | 1-(3-fluoro-4-{4-morpholin-4-yl-1-[1-(pyridin-3-ylcarbonyl)piperidin-4-yl]-1H- |
| | pyrazolo[3,4-d]pyrimidin-6-yl}phenyl)-3-methylurea |
| 462 | 1-ethyl-3-(3-fluoro-4-{4-morpholin-4-yl-1-[1-(pyridin-3-ylcarbonyl)piperidin-4-yl]- |
| | 1H-pyrazolo[3,4-d]pyrimidin-6-yl}phenyl)urea |
| 463 | 1-(2-fluoroethyl)-3-(3-fluoro-4-{4-morpholin-4-yl-1-[1-(pyridin-3- |
| | ylcarbonyl)piperidin-4-yl]-1H-pyrazolo[3,4-d]pyrimidin-6-yl}phenyl)urea |
| 465 | 1-(2,5-difluoro-4-{4-morpholin-4-yl-1-[1-(pyridin-3-ylcarbonyl)piperidin-4-yl]-1H- |
| | pyrazolo[3,4-d]pyrimidin-6-yl}phenyl)-3-methylurea |
| 466 | 1-(2,5-difluoro-4-{4-morpholin-4-yl-1-[1-(pyridin-3-ylcarbonyl)piperidin-4-yl]-1H- |
| | pyrazolo[3,4-d]pyrimidin-6-yl}phenyl)-3-ethylurea |
| 467 | 1-(2,5-difluoro-4-{4-morpholin-4-yl-1-[1-(pyridin-3-ylcarbonyl)piperidin-4-yl]-1H- |
| | pyrazolo[3,4-d]pyrimidin-6-yl}phenyl)-3-(2-fluoroethyl)urea |
| 469 | 1-(2,5-difluoro-4-{4-morpholin-4-yl-1-[1-(pyridin-3-ylcarbonyl)piperidin-4-yl]-1H- |
| | pyrazolo[3,4-d]pyrimidin-6-yl}phenyl)-3-phenylurea |
| 470 | 1-methyl-3-(4-{4-morpholin-4-yl-1-[1-(2,2,2-trifluoroethyl)piperidin-4-yl]-1H- |
| | pyrazolo[3,4-d]pyrimidin-6-yl}phenyl)urea |
| 472 | 1-{4-[1-(1-acetylpiperidin-4-yl)-4-morpholin-4-yl-1H-pyrazolo[3,4-d]pyrimidin-6- |
| | yl]phenyl}-3-methylurea |
| 474 | 1-(4-{1-[1-(methoxyacetyl)piperidin-4-yl]-4-morpholin-4-yl-1H-pyrazolo[3,4- |
| | d]pyrimidin-6-yl}phenyl)-3-methylurea |
| 475 | 1-{4-[1-(1-isobutyrylpiperidin-4-yl)-4-morpholin-4-yl-1H-pyrazolo[3,4- |
| | d]pyrimidin-6-yl]phenyl}-3-methylurea |
| 477 | N-methyl-4-(6-{4-[(methylcarbamoyl)amino]phenyl}-4-morpholin-4-yl-1H- |
| | pyrazolo[3,4-d]pyrimidin-1-yl)piperidine-1-carboxamide |

In another aspect, the invention provides compounds of the Formula III:

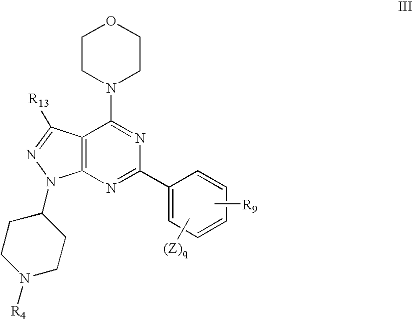

and pharmaceutically acceptable salts, hydrates, and solvates thereof,
wherein:

R₄, R₉, R₁₀, R₁₁, R₁₂, R₁₃, Z and q are as defined above for the compounds of Formula III;

In one embodiment, R₄ is —H.

In one embodiment, R₄ is optionally substituted C₁-C₆alkyl.

In one embodiment, R₄ is —C(O)alkyl.

In one embodiment, R₄ is —C(O)alkoxy.

In one embodiment, R₄ is —C(O)NR₅R₆.

In one embodiment, R₄ is

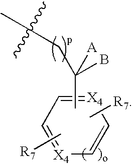

In one embodiment, p is 0.

In one embodiment, p is 1.

In one embodiment, one of A and B is hydrogen.

In another embodiment, A and B are both hydrogen.

In another embodiment, A and B together form a carbonyl.

In another embodiment, one X₄ is —CH—.

In another embodiment, one X₄ is —N—.

In another embodiment, one X₄ is —O—.

In another embodiment, one X₄ is —N⁺(O⁻)—.

In another embodiment, o is 1.

In another embodiment, o is 0.

In one embodiment, R₁₃ is hydrogen.

In one embodiment, R₁₃ is halogen.

In one embodiment, R₁₃ is optionally substituted C₁-C₆alkyl.

In one embodiment, R₁₃ is optionally substituted C₆-C₁₄aryl.

In one embodiment, R₁₃ is optionally substituted C₁-C₉heteroaryl.

In one embodiment, R₅ and R₆ are each independently —H, optionally substituted alkyl, optionally substituted C₆-C₁₄aryl, or optionally substituted C₁-C₉heteroaryl.

In another embodiment, R₅ and R₆ are taken together with the —N— form a nitrogen containing 3 to 7 membered heterocycle wherein up to two of the carbon atoms of the heterocycle may be substituted with —N(R₈)—, —O—, or —S(O)ₙ,

In one embodiment, R₇ is —H.

In one embodiment, R₇ is —OH.

In one embodiment, R₇ is halogen.

In one embodiment, R₇ is optionally substituted alkyl.

In one embodiment, R₇ is optionally substituted alkoxy.

In one embodiment, R₇ is optionally substituted acyl.

In one embodiment, R₇ is optionally substituted amine.

In one embodiment, R₇ is optionally substituted amide.

In one embodiment, R₇ is —CN.

In one embodiment, there is one R₇.

In one embodiment, there are more than one R₇.

In one embodiment, R₈ is optionally substituted C₁-C₆alkyl.

In one embodiment, R₈ is optionally substituted —C(O)—C₁-C₆alkyl.

In one embodiment, R₈ is —C(O)NR₅R₆.

In one embodiment, R₈ is —C(O)OC₁-C₆alkyl.

In one embodiment, R₉ is —OH

In one embodiment, R₉ is —NHC(O)NR₁₀R₁₁.

In one embodiment, R₉ is —NHC(O))R₁₂.

In one embodiment, R₁₀ and R₁₁ are each independently —H, —OH, optionally substituted C₁-C₆alkoxy, optionally substituted C₆-C₁₄aryl, optionally substituted C₁-C₉heteroaryl, optionally substituted —C₃-C₈carbocycle, or optionally substituted —C₁-C₆alkyl.

In one embodiment, R₁₀ and R₁₁ are taken together with the nitrogen to which they are attached to form a nitrogen containing 3- to 7- membered monocyclic C₁-C₆heterocycle.

In another embodiment, the nitrogen containing 3- to 7- membered monocyclic C₁-C₆heterocycle has up to two of the carbon atoms of the heterocycle substituted with —N(R₈)—, —O—, or —S(O)ₙ.

In another embodiment, R₁₂ is optionally substituted —C₁-C₆alkyl.

In another embodiment, R₁₂ is optionally substituted —C₁-C₆ alkoxy.

In another embodiment, Z is chlorine.

In another embodiment, Z is fluorine.

In a further embodiment, A and B together form a carbonyl, R₇ is hydrogen, and R₉ is —NHC(O)NR₁₀R₁₁.

Illustrative compounds of Formula III are exemplified by the following compounds:

| Example | Name |
|---|---|
| 195 | 1-{1-[(2-chloropyridin-3-yl)carbonyl]piperidin-4-yl}-6-(1H-indol-5-yl)-4-morpholin-4-yl- |
| | 1H-pyrazolo[3,4-d]pyrimidine |
| 204 | 3-(4-morpholin-4-yl-1H-pyrazolo[3,4-d]pyrimidin-6-yl)phenol |
| 206 | N-{4-[1-(1-benzylpiperidin-4-yl)-4-morpholin-4-yl-1H-pyrazolo[3,4-d]pyrimidin-6- |
| | yl]phenyl}-N′-methylurea |
| 209 | methyl (4-{4-morpholin-4-yl-1-[1-(pyridin-3-ylmethyl)piperidin-4-yl]-1H-pyrazolo[3,4- |
| | d]pyrimidin-6-yl}phenyl)carbamate |
| 216 | 1-butyl-3-(4-{4-morpholin-4-yl-1-[1-(pyridin-3-ylcarbonyl)piperidin-4-yl]-1H- |
| | pyrazolo[3,4-d]pyrimidin-6-yl}phenyl)urea |
| 222 | methyl (4-{4-morpholin-4-yl-1-[1-(pyridin-3-ylmethyl)piperidin-4-yl]-1H-pyrazolo[3,4- |
| | d]pyrimidin-6-yl}phenyl)carbamate |
| 223 | methyl (4-{4-morpholin-4-yl-1-[1-(pyridin-3-ylcarbonyl)piperidin-4-yl]-1H-pyrazolo[3,4- |
| | d]pyrimidin-6-yl}phenyl)carbamate |
| 225 | 1-methyl-3-(4-{4-morpholin-4-yl-1-[1-(pyridin-3-ylmethyl)piperidin-4-yl]-1H- |
| | pyrazolo[3,4-d]pyrimidin-6-yl}phenyl)urea |
| 226 | 1-methyl-3-(4-{4-morpholin-4-yl-1-[1-(pyridin-3-ylcarbonyl)piperidin-4-yl]-1H- |
| | pyrazolo[3,4-d]pyrimidin-6-yl}phenyl)urea |
| 253 | 1-{4-[1-(1-benzylpiperidin-4-yl)-4-morpholin-4-yl-1H-pyrazolo[3,4-d]pyrimidin-6- |
| | yl]phenyl}-3-ethylurea |
| 261 | 1-{4-[1-(1-benzylpiperidin-4-yl)-4-morpholin-4-yl-1H-pyrazolo[3,4-d]pyrimidin-6- |
| | yl]phenyl}-3-pyridin-3-ylurea |
| 262 | 1-{4-[1-(1-benzylpiperidin-4-yl)-4-morpholin-4-yl-1H-pyrazolo[3,4-d]pyrimidin-6- |
| | yl]phenyl}-3-(cyclopropylmethyl)urea |
| 265 | 1-{4-[1-(1-benzylpiperidin-4-yl)-4-morpholin-4-yl-1H-pyrazolo[3,4-d]pyrimidin-6- |
| | yl]phenyl}-3-(2-hydroxyethyl)urea |
| 294 | 1-(2-fluoroethyl)-3-[4-(4-morpholin-4-yl-1-phenyl-1H-pyrazolo[3,4-d]pyrimidin-6- |
| | yl)phenyl]urea |
| 301 | 1-(4-{4-morpholin-4-yl-1-[1-(pyridin-3-ylcarbonyl)piperidin-4-yl]-1H-pyrazolo[3,4- |
| | d]pyrimidin-6-yl}phenyl)urea |
| 302 | 1-(4-{4-morpholin-4-yl-1-[1-(pyridin-3-ylcarbonyl)piperidin-4-yl]-1H-pyrazolo[3,4- |
| | d]pyrimidin-6-yl}phenyl)-3-pyridin-3-ylurea |
| 303 | 1-(2-fluoroethyl)-3-(4-{4-morpholin-4-yl-1-[1-(pyridin-3-ylcarbonyl)piperidin-4-yl]-1H- |
| | pyrazolo[3,4-d]pyrimidin-6-yl}phenyl)urea |
| 304 | 1-(2-hydroxyethyl)-3-(4-{4-morpholin-4-yl-1-[1-(pyridin-3-ylcarbonyl)piperidin-4-yl]-1H- |
| | pyrazolo[3,4-d]pyrimidin-6-yl}phenyl)urea |
| 307 | 1-ethyl-3-(4-{4-morpholin-4-yl-1-[1-(pyridin-3-ylcarbonyl)piperidin-4-yl]-1H-pyrazolo[3,4- |
| | d]pyrimidin-6-yl}phenyl)urea |
| 314 | 1-(4-{4-morpholin-4-yl-1-[1-(pyridin-3-ylmethyl)piperidin-4-yl]-1H-pyrazolo[3,4- |
| | d]pyrimidin-6-yl}phenyl)-3-pyridin-3-ylurea |
| 315 | 1-(2-fluoroethyl)-3-(4-{4-morpholin-4-yl-1-[1-(pyridin-3-ylmethyl)piperidin-4-yl]-1H- |
| | pyrazolo[3,4-d]pyrimidin-6-yl}phenyl)urea |
| 316 | 1-(2-hydroxyethyl)-3-(4-{4-morpholin-4-yl-1-[1-(pyridin-3-ylmethyl)piperidin-4-yl]-1H- |
| | pyrazolo[3,4-d]pyrimidin-6-yl}phenyl)urea |
| 318 | 1-ethyl-3-(4-{4-morpholin-4-yl-1-[1-(pyridin-3-ylmethyl)piperidin-4-yl]-1H-pyrazolo[3,4- |
| | d]pyrimidin-6-yl}phenyl)urea |
| 321 | 1-{4-[1-(1,4-dioxaspiro[4.5]dec-8-yl)-4-morpholin-4-yl-1H-pyrazolo[3,4-d]pyrimidin-6- |
| | yl]phenyl}-3-methylurea |
| 322 | 1-methyl-3-{4-[4-morpholin-4-yl-1-(4-oxocyclohexyl)-1H-pyrazolo[3,4-d]pyrimidin-6- |
| | yl]phenyl}urea |
| 323 | 1-{4-[1-(4-hydroxycyclohexyl)-4-morpholin-4-yl-1H-pyrazolo[3,4-d]pyrimidin-6- |
| | yl]phenyl}-3-methylurea |
| 326 | 1-{4-[1-(1-benzylpiperidin-4-yl)-4-morpholin-4-yl-1H-pyrazolo[3,4-d]pyrimidin-6- |
| | yl]phenyl}-3-(1H-imidazol-2-yl)urea |
| 328 | 1-methyl-3-[4-(4-morpholin-4-yl-1-{1-[(1-oxidopyridin-3-yl)carbonyl]piperidin-4-yl}-1H- |
| | pyrazolo[3,4-d]pyrimidin-6-yl)phenyl]urea |
| 329 | 1-methyl-3-[4-(1-{1-[(2-methylpyridin-3-yl)carbonyl]piperidin-4-yl}-4-morpholin-4-yl-1H- |
| | pyrazolo[3,4-d]pyrimidin-6-yl)phenyl]urea |
| 330 | 1-[4-(1-{1-[(6-methoxypyridin-3-yl)methyl]piperidin-4-yl}-4-morpholin-4-yl-1H- |
| | pyrazolo[3,4-d]pyrimidin-6-yl)phenyl]-3-methylurea |
| 331 | 1-methyl-3-(4-{4-morpholin-4-yl-1-[1-(pyridin-2-ylmethyl)piperidin-4-yl]-1H- |
| | pyrazolo[3,4-d]pyrimidin-6-yl}phenyl)urea |
| 332 | 2-[4-(6-{4-[(methylcarbamoyl)amino]phenyl}-4-morpholin-4-yl-1H-pyrazolo[3,4- |
| | d]pyrimidin-1-yl)piperidin-1-yl]acetamide |
| 333 | 1-methyl-3-(4-{4-morpholin-4-yl-1-[1-(2-oxo-2-phenylethyl)piperidin-4-yl]-1H- |
| | pyrazolo[3,4-d]pyrimidin-6-yl}phenyl)urea |
| 336 | 1-{4-[1-(1-benzoylpiperidin-4-yl)-4-morpholin-4-yl-1H-pyrazolo[3,4-d]pyrimidin-6- |
| | yl]phenyl}-3-methylurea |
| 337 | tert-butyl 4-(6-{4-[(methylcarbamoyl)amino]phenyl}-4-morpholin-4-yl-1H-pyrazolo[3,4- |
| | d]pyrimidin-1-yl)piperidine-1-carboxylate |
| 338 | 1-methyl-3-[4-(4-morpholin-4-yl-1-piperidin-4-yl-1H-pyrazolo[3,4-d]pyrimidin-6- |
| | yl)phenyl]urea |
| 339 | 1-(4-{4-morpholin-4-yl-1-[1-(pyridin-3-ylmethyl)piperidin-4-yl]-1H-pyrazolo[3,4- |
| | d]pyrimidin-6-yl}phenyl)-3-phenylurea |
| 340 | 1-(4-{4-morpholin-4-yl-1-[1-(pyridin-3-ylmethyl)piperidin-4-yl]-1H-pyrazolo[3,4- |
| | d]pyrimidin-6-yl}phenyl)-3-pyridin-4-ylurea |
| 341 | 1-(4-{4-morpholin-4-yl-1-[1-(pyridin-3-ylmethyl)piperidin-4-yl]-1H-pyrazolo[3,4- |
| | d]pyrimidin-6-yl}phenyl)-3-pyridin-2-ylurea |
| 343 | 1-(4-{4-morpholin-4-yl-1-[1-(pyridin-3-ylcarbonyl)piperidin-4-yl]-1H-pyrazolo[3,4- |
| | d]pyrimidin-6-yl}phenyl)-3-phenylurea |
| 344 | 1-(4-{4-morpholin-4-yl-1-[1-(pyridin-3-ylcarbonyl)piperidin-4-yl]-1H-pyrazolo[3,4- |
| | d]pyrimidin-6-yl}phenyl)-3-pyridin-4-ylurea |
| 345 | 1-(4-{4-morpholin-4-yl-1-[1-(pyridin-3-ylcarbonyl)piperidin-4-yl]-1H-pyrazolo[3,4- |
| | d]pyrimidin-6-yl}phenyl)-3-pyridin-2-ylurea |
| 350 | 1-(2-fluoro-4-{4-morpholin-4-yl-1-[1-(pyridin-3-ylmethyl)piperidin-4-yl]-1H-pyrazolo[3,4- |
| | d]pyrimidin-6-yl}phenyl)-3-methylurea |
| 351 | 1-(2-fluoro-4-{4-morpholin-4-yl-1-[1-(pyridin-3-ylmethyl)piperidin-4-yl]-1H-pyrazolo[3,4- |
| | d]pyrimidin-6-yl}phenyl)-3-phenylurea |
| 354 | 1-(4-{1-[1-(2-fluorobenzoyl)piperidin-4-yl]-4-morpholin-4-yl-1H-pyrazolo[3,4- |
| | d]pyrimidin-6-yl}phenyl)-3-methylurea |
| 355 | 1-(4-{1-[1-(2-chlorobenzoyl)piperidin-4-yl]-4-morpholin-4-yl-1H-pyrazolo[3,4- |
| | d]pyrimidin-6-yl}phenyl)-3-methylurea |
| 356 | 1-methyl-3-(4-{1-[1-(2-methylbenzoyl)piperidin-4-yl]-4-morpholin-4-yl-1H-pyrazolo[3,4- |
| | d]pyrimidin-6-yl}phenyl)urea |
| 357 | 1-(4-{1-[1-(3-fluorobenzoyl)piperidin-4-yl]-4-morpholin-4-yl-1H-pyrazolo[3,4- |
| | d]pyrimidin-6-yl}phenyl)-3-methylurea |
| 358 | 1-(4-{1-[1-(3-chlorobenzoyl)piperidin-4-yl]-4-morpholin-4-yl-1H-pyrazolo[3,4- |
| | d]pyrimidin-6-yl}phenyl)-3-methylurea |
| 359 | 1-methyl-3-[4-(4-morpholin-4-yl-1-{1-[3-(trifluoromethyl)benzoyl]piperidin-4-yl}-1H- |
| | pyrazolo[3,4-d]pyrimidin-6-yl)phenyl]urea |
| 360 | 1-(4-{1-[1-(4-bromobenzoyl)piperidin-4-yl]-4-morpholin-4-yl-1H-pyrazolo[3,4- |
| | d]pyrimidin-6-yl}phenyl)-3-methylurea |
| 361 | 1-(4-{1-[1-(4-fluorobenzoyl)piperidin-4-yl]-4-morpholin-4-yl-1H-pyrazolo[3,4- |
| | d]pyrimidin-6-yl}phenyl)-3-methylurea |
| 362 | 1-(4-{1-[1-(4-chlorobenzoyl)piperidin-4-yl]-4-morpholin-4-yl-1H-pyrazolo[3,4- |
| | d]pyrimidin-6-yl}phenyl)-3-methylurea |
| 363 | 1-(4-{1-[1-(4-methoxybenzoyl)piperidin-4-yl]-4-morpholin-4-yl-1H-pyrazolo[3,4- |
| | d]pyrimidin-6-yl}phenyl)-3-methylurea |
| 364 | 1-(4-{1-[1-(3-methoxybenzoyl)piperidin-4-yl]-4-morpholin-4-yl-1H-pyrazolo[3,4- |
| | d]pyrimidin-6-yl}phenyl)-3-methylurea |
| 365 | 1-(4-{1-[1-(2-methoxybenzoyl)piperidin-4-yl]-4-morpholin-4-yl-1H-pyrazolo[3,4- |
| | d]pyrimidin-6-yl}phenyl)-3-methylurea |
| 366 | 1-methyl-3-(4-{1-[1-(3-methylbenzoyl)piperidin-4-yl]-4-morpholin-4-yl-1H-pyrazolo[3,4- |
| | d]pyrimidin-6-yl}phenyl)urea |
| 367 | 1-methyl-3-(4-{1-[1-(4-methylbenzoyl)piperidin-4-yl]-4-morpholin-4-yl-1H-pyrazolo[3,4- |
| | d]pyrimidin-6-yl}phenyl)urea |
| 373 | 1-(2-fluoro-4-{4-morpholin-4-yl-1-[1-(pyridin-3-ylmethyl)piperidin-4-yl]-1H-pyrazolo[3,4- |
| | d]pyrimidin-6-yl}phenyl)-3-pyridin-3-ylurea |
| 378 | 1-{4-[1-(1-benzoylpiperidin-4-yl)-4-morpholin-4-yl-1H-pyrazolo[3,4-d]pyrimidin-6- |
| | yl]phenyl}-3-phenylurea |
| 379 | 1-{4-[1-(1-benzoylpiperidin-4-yl)-4-morpholin-4-yl-1H-pyrazolo[3,4-d]pyrimidin-6- |
| | yl]phenyl}-3-pyridin-3-ylurea |
| 380 | 1-{4-[1-(1-benzoylpiperidin-4-yl)-4-morpholin-4-yl-1H-pyrazolo[3,4-d]pyrimidin-6- |
| | yl]phenyl}-3-(2-fluoroethyl)urea |
| 381 | 1-{4-[1-(1-benzoylpiperidin-4-yl)-4-morpholin-4-yl-1H-pyrazolo[3,4-d]pyrimidin-6- |
| | yl]phenyl}-3-ethylurea |
| 382 | 1-{4-[1-(1-benzoylpiperidin-4-yl)-4-morpholin-4-yl-1H-pyrazolo[3,4-d]pyrimidin-6- |
| | yl]phenyl}urea |
| 384 | 1-{4-[1-(1-benzoylpiperidin-4-yl)-4-morpholin-4-yl-1H-pyrazolo[3,4-d]pyrimidin-6- |
| | yl]phenyl}-3-pyridin-4-ylurea |
| 385 | 1-{4-[1-(1-benzoylpiperidin-4-yl)-4-morpholin-4-yl-1H-pyrazolo[3,4-d]pyrimidin-6- |
| | yl]phenyl}-3-(2-hydroxyethyl)urea |
| 386 | 1-{4-[1-(1-benzoylpiperidin-4-yl)-4-morpholin-4-yl-1H-pyrazolo[3,4-d]pyrimidin-6- |
| | yl]phenyl}-3-[2-(methylamino)ethyl]urea |
| 387 | 1-[4-(1-{1-[(6-fluoropyridin-3-yl)methyl]piperidin-4-yl}-4-morpholin-4-yl-1H- |
| | pyrazolo[3,4-d]pyrimidin-6-yl)phenyl]-3-methylurea |
| 388 | 1-[4-(1-{1-[(6-chloropyridin-3-yl)methyl]piperidin-4-yl}-4-morpholin-4-yl-1H- |
| | pyrazolo[3,4-d]pyrimidin-6-yl)phenyl]-3-methylurea |
| 389 | 1-[4-(1-{1-[(6-bromopyridin-3-yl)methyl]piperidin-4-yl}-4-morpholin-4-yl-1H- |
| | pyrazolo[3,4-d]pyrimidin-6-yl)phenyl]-3-methylurea |
| 390 | 1-[4-(1-{1-[(2-chloropyridin-3-yl)methyl]piperidin-4-yl}-4-morpholin-4-yl-1H- |
| | pyrazolo[3,4-d]pyrimidin-6-yl)phenyl]-3-methylurea |
| 391 | 1-[4-(1-{1-[(4-methoxypyridin-3-yl)methyl]piperidin-4-yl}-4-morpholin-4-yl-1H- |
| | pyrazolo[3,4-d]pyrimidin-6-yl)phenyl]-3-methylurea |
| 392 | 1-[4-(1-{1-[(5-fluoropyridin-3-yl)methyl]piperidin-4-yl}-4-morpholin-4-yl-1H- |
| | pyrazolo[3,4-d]pyrimidin-6-yl)phenyl]-3-methylurea |
| 393 | 1-[4-(1-{1-[(5-bromopyridin-3-yl)methyl]piperidin-4-yl}-4-morpholin-4-yl-1H- |
| | pyrazolo[3,4-d]pyrimidin-6-yl)phenyl]-3-methylurea |
| 394 | 1-(4-{1-[1-(4-chlorobenzyl)piperidin-4-yl]-4-morpholin-4-yl-1H-pyrazolo[3,4-d]pyrimidin- |
| | 6-yl}phenyl)-3-methylurea |
| 395 | 1-(4-{1-[1-(3-chlorobenzyl)piperidin-4-yl]-4-morpholin-4-yl-1H-pyrazolo[3,4-d]pyrimidin- |
| | 6-yl}phenyl)-3-methylurea |
| 396 | 1-(4-{1-[1-(2-chlorobenzyl)piperidin-4-yl]-4-morpholin-4-yl-1H-pyrazolo[3,4-d]pyrimidin- |
| | 6-yl}phenyl)-3-methylurea |
| 399 | 1-(4-{1-[1-(2-hydroxybenzyl)piperidin-4-yl]-4-morpholin-4-yl-1H-pyrazolo[3,4- |
| | d]pyrimidin-6-yl}phenyl)-3-methylurea |
| 400 | 1-(4-{1-[1-(3-methoxybenzyl)piperidin-4-yl]-4-morpholin-4-yl-1H-pyrazolo[3,4- |
| | d]pyrimidin-6-yl}phenyl)-3-methylurea |
| 403 | 1-methyl-3-{4-[4-morpholin-4-yl-1-(tetrahydro-2H-pyran-4-yl)-1H-pyrazolo[3,4- |
| | d]pyrimidin-6-yl]phenyl}urea |
| 404 | 1-(4-{4-morpholin-4-yl-1-[1-(pyridin-3-ylmethyl)piperidin-4-yl]-1H-pyrazolo[3,4- |
| | d]pyrimidin-6-yl}phenyl)-3-(2-thienyl)urea |
| 405 | 1-cyclopropyl-3-(4-{4-morpholin-4-yl-1-[1-(pyridin-3-ylmethyl)piperidin-4-yl]-1H- |
| | pyrazolo[3,4-d]pyrimidin-6-yl}phenyl)urea |
| 413 | 1-(4-{4-morpholin-4-yl-1-[1-(pyridin-3-ylcarbonyl)piperidin-4-yl]-1H-pyrazolo[3,4- |
| | d]pyrimidin-6-yl}phenyl)-3-(2-thienyl)urea |
| 414 | 1-(4-{4-morpholin-4-yl-1-[1-(pyridin-3-ylcarbonyl)piperidin-4-yl]-1H-pyrazolo[3,4- |
| | d]pyrimidin-6-yl}phenyl)-3-(3-thienyl)urea |
| 415 | 1-cyclopropyl-3-(4-{4-morpholin-4-yl-1-[1-(pyridin-3-ylcarbonyl)piperidin-4-yl]-1H- |
| | pyrazolo[3,4-d]pyrimidin-6-yl}phenyl)urea |
| 417 | 1-{4-[1-(1-isonicotinoylpiperidin-4-yl)-4-morpholin-4-yl-1H-pyrazolo[3,4-d]pyrimidin-6- |
| | yl]phenyl}-3-methylurea |
| 418 | 1-methyl-3-(4-{4-morpholin-4-yl-1-[1-(pyridin-2-ylcarbonyl)piperidin-4-yl]-1H- |
| | pyrazolo[3,4-d]pyrimidin-6-yl}phenyl)urea |
| 419 | 1-methyl-3-[4-(1-{1-[(4-methylpyridin-3-yl)carbonyl]piperidin-4-yl}-4-morpholin-4-yl-1H- |
| | pyrazolo[3,4-d]pyrimidin-6-yl)phenyl]urea |
| 420 | 1-methyl-3-[4-(1-{1-[(6-methylpyridin-3-yl)carbonyl]piperidin-4-yl}-4-morpholin-4-yl-1H- |
| | pyrazolo[3,4-d]pyrimidin-6-yl)phenyl]urea |
| 421 | 1-[4-(1-{1-[(6-fluoropyridin-3-yl)carbonyl]piperidin-4-yl}-4-morpholin-4-yl-1H- |
| | pyrazolo[3,4-d]pyrimidin-6-yl)phenyl]-3-methylurea |
| 422 | 1-methyl-3-(4-{4-morpholin-4-yl-1-[1-(pyrazin-2-ylcarbonyl)piperidin-4-yl]-1H- |
| | pyrazolo[3,4-d]pyrimidin-6-yl}phenyl)urea |
| 423 | 1-(4-{1-[1-(3-acetylbenzoyl)piperidin-4-yl]-4-morpholin-4-yl-1H-pyrazolo[3,4-d]pyrimidin- |
| | 6-yl}phenyl)-3-methylurea |
| 424 | 1-[4-(1-{1-[(6-chloropyridin-3-yl)carbonyl]piperidin-4-yl}-4-morpholin-4-yl-1H- |
| | pyrazolo[3,4-d]pyrimidin-6-yl)phenyl]-3-methylurea |
| 473 | N,N-dimethyl-4-(6-{4-[(methylcarbamoyl)amino]phenyl}-4-morpholin-4-yl-1H- |
| | pyrazolo[3,4-d]pyrimidin-1-yl)piperidine-1-carboxamide |
| 476 | methyl 4-(6-{4-[(methylcarbamoyl)amino]phenyl}-4-morpholin-4-yl-1H-pyrazolo[3,4- |
| | d]pyrimidin-1-yl)piperidine-1-carboxylate |

In another aspect, the invention provides compounds of Formula IIIa:

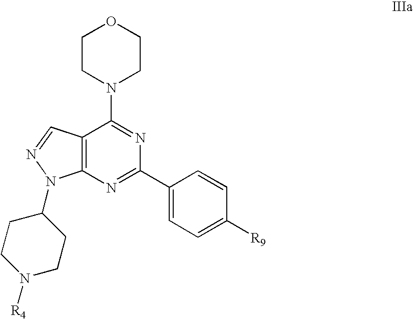

and pharmaceutically acceptable salts, hydrates, and solvates thereof,

wherein R₄, R₅, R₆, R₈, R₉, R₁₀, R₁₁, R₁₂, R₁₅, and n are as defined above for compounds of Formula IIIa.

In one embodiment, R₄ is optionally substituted —C(O)alkoxy.

In one embodiment, R₄ is optionally substituted —C(O)NR₅R₆.

In one embodiment, R₄ is —C(O)OC₂-C₁₀alkyne.

In one embodiment, R₄ is

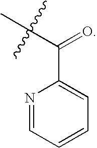

In one embodiment, R₄ is

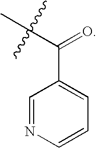

In one embodiment, R₄ is

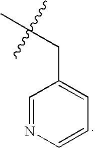

In one embodiment, R₄ is

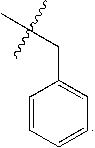

In one embodiment, R₅ is hydrogen.

In one embodiment, R₆ is —C₁-C₆alkyl.

In one embodiment, R₆ is optionally substituted C₆-C₁₄aryl.

In one embodiment, R₆ is —C(O)—R₁₅.

In one embodiment, R₅ and R₆ are taken together with the nitrogen to which they are attached to form a nitrogen containing 3 to 7 membered monocyclic C₁-C₆heterocycle.

In one embodiment, R₉ is —NHC(O)NR₁₀R₁₁.

In one embodiment, R₉ is —NHC(O)OR₁₂;

In one embodiment, R₁₀ is hydrogen.

In one embodiment, R₁₁ is —OH.

In one embodiment, R₁₁ is optionally substituted C₁-C₆alkoxy.

In one embodiment, R₁₁ is optionally substituted C₆-C₁₄aryl.

In one embodiment, R₁₁ is optionally substituted C₁-C₉heteroaryl.

In one embodiment, R₁₁ is optionally substituted —C₃-C₈carbocycle.

In one embodiment, R₁₁ is cyclopropyl.

In one embodiment, R₁₁ is optionally substituted —C₁-C₆alkyl.

In one embodiment, R₁₂ is methyl.

In one embodiment, R₁₂ is ethyl.

In one embodiment, R₁₂ is propyl.

In one embodiment R₁₅ is optionally substituted C₁-C₆alkyl, optionally substituted C₆-C₁₄aryl, optionally substituted C₁-C₉heteroaryl, optionally substituted (C₁-C₆alkyl)amino, or optionally substituted (C₆-C₁₄aryl)amino.

In another aspect, the invention provides compounds of Formula Ia:

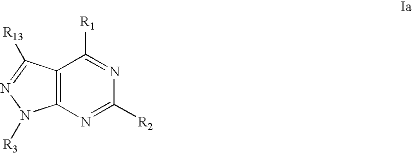

or a pharmaceutically acceptable salt or tautomer thereof, wherein R₁, R₂, R₃, and R₁₃ are as defined above for the compounds of Formula Ia.
except that 4-(4-morpholinyl)-1-phenyl-6-[3-(trifluoromethyl)phenyl]-1H-pyrazolo[3,4-d]pyrimidine is excluded.

In one embodiment, X₅ is —O—.

In one embodiment, R₂ is C₆-C₁₄aryl optionally independently substituted with from 1 to 3 substituents as specified in Formula (Ia).

In one embodiment, R₂ is C₆-C₁₄aryl substituted by —NHC(O)NHNR¹⁶R¹⁷.

In one embodiment, R₂ is C₆-C₁₄aryl substituted by —NHC(O)OR¹⁸.

In one embodiment, R₃ is hydrogen.

In one embodiment, R₃ is C₆-C₁₄aryl.

In one embodiment, R₃ is monocyclic C₁-C₆heterocycle optionally independently substituted with from 1 to 3 substituents as specified in Formula (Ia).

In one embodiment, the monocyclic C₁-C₆heterocycle is a piperidine.

In one embodiment, the C4 of the piperidine ring is directly bonded to N-1 of the 1H-pyrazolo[3,4-d]pyrimidine ring of Formula (Ib).

In one embodiment, the piperidine nitrogen is further substituted with a substituent selected from:
a) C₁-C₈acyl, wherein the C₁-C₈acyl is optionally substituted with from 1 to 3 substituents independently selected from:
   (i) hydroxyl,
   (ii) CN,
   (iii) C₁-C₆alkoxy,
   (iv) C₁-C₆alkyl,
   (v) C₁-C₈acyl,
   (vi) NH₂,
   (vii) (C₁-C₆alkyl)amino,
   (viii) di(C₁-C₆alkyl)amino,
   (ix) CO₂H,
   (x) (C₁-C₆alkoxy)carbonyl,
   (xi) C₁-C₆perfluoroalkyl,
   (xii) and halogen;
b) C₁-C₆alkyl optionally substituted with from 1 to 3 substituents independently selected from:
   (i) C₃-C₈cycloalkyl,
   (ii) C₁-C₆alkoxy,
   (iii) C₁-C₈acyl,
   (iv) CN,
   (v) (C₁-C₆alkoxy)carbonyl,
   (vi) CO₂H,
   (vii) hydroxyl,
   (viii) C₁-C₉heterocycle,
   (ix) or H₂NC(O)—;
c) C₁-C₆perfluoroalkyl;
d) C₂-C₆alkenyl;
e) heteroaryl(C₁-C₆alkyl) wherein the ring portion of the heteroaryl(C₁-C₆alkyl) group is optionally substituted with from 1 to 3 substituents independently selected from:
   (i) C₁-C₆alkylC(O)NH—,
   (ii) C₁-C₆alkoxy,
   (iii) halogen,
   (iv) NH₂,
   (v) and C₁-C₆alkyl;
f) (C₆-C₁₄aryl)alkyl, wherein the ring portion of the (C₆-C₁₄aryl)alkyl group is optionally substituted by 1 to 3 substituents independently selected from:
   (i) halogen,
   (ii) C₁-C₆alkyl,
   (iii) NH₂,
   (iv) (C₁-C₆alkyl)amino,
   (v) di(C₁-C₆alkyl)amino,
   (vi) hydroxyl,
   (vii) C₁-C₆alkoxy,
   (viii) C₁-C₈acyl,
   (ix) and C₁-C₉heteroaryl;
g) HC(O)—;
h) C₁-C₆perfluoroalkyl;
i) —S(O)_{q}—(C₁-C₆alkyl);
j) —S(O)_{q}-aryl;
k) R¹⁹R²⁰NC(O);
l) (C₁-C₉heteroaryl)-NH—C(S)—;
m) (C₁-C₆alkyl)-NH—C(S)—;
n) (C₁-C₆alkyl)-S—C(O)—;
o) (C₆-C₁₄aryloxy)carbonyl;
p) (C₂-C₆alkenyloxy)carbonyl;
q) (C₂-C₆alkynyloxy)carbonyl;
r) and (C₁-C₆alkoxy)carbonyl optionally substituted with from 1 to 3 substituents independently selected from:
   (i) C₁-C₆alkoxy,
   (ii) halogen,
   (iii) C₆-C₁₄aryl,
   (iv) NH₂,
   (v) (C₁-C₆alkyl)amino-,
   (vi) di(C₁-C₆alkyl)amino-;
   (vii) and C₁-C₆alkyl.

In one embodiment, R₃ is monocyclic C₁-C₆heterocycle optionally substituted with from 1 to 3 substituents as specified in Formula Ia and X₅ is —O—.

In one embodiment, R₂ is C₆-C₁₄aryl optionally independently substituted with from 1 to 3 substituents as specified in Formula Ia and R₃ is monocyclic C₁-C₆heterocycle optionally independently substituted with from 1 to 3 substituents as specified in Formula (Ia).

In one embodiment, R₂ is C₆-C₁₄aryl substituted by —NHC(O)NHNR₁₆R₁₇ and R₃ is monocyclic C₁-C₆heterocycle optionally substituted with from 1 to 3 substituents as specified in Formula Ia.

In one embodiment, R₂ is C₆-C₁₄aryl substituted by —NHC(O)OR¹⁸ and R₃ is monocyclic C₁-C₆heterocycle optionally substituted with from 1 to 3 substituents as specified in Formula Ia.

In one embodiment, R₂ is C₁-C₉heteroaryl optionally independently substituted with from 1 to 3 substituents as specified in Formula Ia and R₃ is monocyclic C₁-C₆heterocycle optionally substituted with from 1 to 3 substituents as specified in Formula Ia.

Illustrative compounds of Formula Ia are exemplified by the following compounds:

| Example | Name |
|---|---|
| 481 | methyl {3-[1-(1-benzylpiperidin-4-yl)-4-morpholin-4-yl-1H- |
| | pyrazolo[3,4-d]pyrimidin-6-yl]phenyl}carbamate |
| 482 | N-{3-[1-(1-benzylpiperidin-4-yl)-4-morpholin-4-yl-1H-pyrazolo[3,4- |
| | d]pyrimidin-6-yl]phenyl}-N′-methylurea |
| 483 | N-{3-[1-(1-benzylpiperidin-4-yl)-4-morpholin-4-yl-1H-pyrazolo[3,4- |
| | d]pyrimidin-6-yl]phenyl}urea |
| 484 | 3-[1-(1-benzylpiperidin-4-yl)-4-morpholin-4-yl-1H-pyrazolo[3,4-d]pyrimidin- |
| | 6-yl]quinoline |
| 485 | N-{4-[1-(1-benzylpiperidin-4-yl)-4-morpholin-4-yl-1H-pyrazolo[3,4- |
| | d]pyrimidin-6-yl]phenyl}formamide |
| 486 | 4-[1-(1-benzylpiperidin-4-yl)-4-morpholin-4-yl-1H-pyrazolo[3,4- |
| | d]pyrimidin-6-yl]phenol |
| 487 | 4-{4-[6-(1H-indol-5-yl)-4-morpholin-4-yl-1H-pyrazolo[3,4- |
| | d]pyrimidin-1-yl]piperidin-1-yl}-N,N-dimethyl-4-oxobut-2-en-1-amine |
| 488 | 6-(1H-indol-5-yl)-1-(1-isopropylpiperidin-4-yl)-4-morpholin-4-yl-1H- |
| | pyrazolo[3,4-d]pyrimidine |
| 489 | 6-(1H-indol-5-yl)-4-morpholin-4-yl-1-[1-(2-phenylethyl)piperidin-4- |
| | yl]-1H-pyrazolo[3,4-d]pyrimidine |
| 490 | 6-(1H-indol-5-yl)-4-morpholin-4-yl-1-[1-(1-phenylethyl)piperidin-4- |
| | yl]-1H-pyrazolo[3,4-d]pyrimidine |
| 491 | 6-(1H-indol-5-yl)-1-[1-(2-methoxyethyl)piperidin-4-yl]-4-morpholin- |
| | 4-yl-1H-pyrazolo[3,4-d]pyrimidine |
| 492 | 6-(1H-indol-5-yl)-4-morpholin-4-yl-1-[1-(phenylacetyl)piperidin-4- |
| | yl]-1H-pyrazolo[3,4-d]pyrimidine |
| 493 | phenyl 4-[6-(1H-indol-5-yl)-4-morpholin-4-yl-1H-pyrazolo[3,4-d]pyrimidin- |
| | 1-yl]piperidine-1-carboxylate |
| 494 | methyl 4-[6-(1H-indol-5-yl)-4-morpholin-4-yl-1H-pyrazolo[3,4- |
| | d]pyrimidin-1-yl]piperidine-1-carboxylate |
| 495 | 2-{4-[6-(1H-indol-5-yl)-4-morpholin-4-yl-1H-pyrazolo[3,4- |
| | d]pyrimidin-1-yl]piperidin-1-yl}acetamide |
| 496 | 2-{4-[6-(1H-indol-5-yl)-4-morpholin-4-yl-1H-pyrazolo[3,4- |
| | d]pyrimidin-1-yl]piperidin-1-yl}ethanol |
| 497 | 3-{4-[6-(1H-indol-5-yl)-4-morpholin-4-yl-1H-pyrazolo[3,4- |
| | d]pyrimidin-1-yl]piperidin-1-yl}propan-1-ol |
| 498 | 1-{4-[1-(1-benzylpiperidin-4-yl)-4-morpholin-4-yl-1H-pyrazolo[3,4- |
| | d]pyrimidin-6-yl]phenyl}urea |
| 499 | 1-{4-[1-(1-benzylpiperidin-4-yl)-4-morpholin-4-yl-1H-pyrazolo[3,4- |
| | d]pyrimidin-6-yl]phenyl}-3-ethylurea |
| 500 | 1-{4-[1-(1-benzylpiperidin-4-yl)-4-morpholin-4-yl-1H-pyrazolo[3,4- |
| | d]pyrimidin-6-yl]phenyl}-3-propylurea |
| 501 | propyl {4-[1-(1-benzylpiperidin-4-yl)-4-morpholin-4-yl-1H- |
| | pyrazolo[3,4-d]pyrimidin-6-yl]phenyl}carbamate |
| 502 | 1-{4-[1-(1-benzylpiperidin-4-yl)-4-morpholin-4-yl-1H-pyrazolo[3,4- |
| | d]pyrimidin-6-yl]phenyl}-3-isopropylurea |
| 503 | 1-{4-[1-(1-benzylpiperidin-4-yl)-4-morpholin-4-yl-1H-pyrazolo[3,4- |
| | d]pyrimidin-6-yl]phenyl}-3-phenylurea |
| 504 | 1-benzyl-3-{4-[1-(1-benzylpiperidin-4-yl)-4-morpholin-4-yl-1H- |
| | pyrazolo[3,4-d]pyrimidin-6-yl]phenyl}urea |
| 505 | 1-{4-[1-(1-benzylpiperidin-4-yl)-4-morpholin-4-yl-1H-pyrazolo[3,4- |
| | d]pyrimidin-6-yl]phenyl}-3-(2-phenylethyl)urea |
| 506 | 1-{4-[1-(1-benzylpiperidin-4-yl)-4-morpholin-4-yl-1H-pyrazolo[3,4- |
| | d]pyrimidin-6-yl]phenyl}-3-(3-phenylpropyl)urea |
| 507 | 1-{4-[1-(1-benzylpiperidin-4-yl)-4-morpholin-4-yl-1H-pyrazolo[3,4- |
| | d]pyrimidin-6-yl]phenyl}-3-pyridin-3-ylurea |
| 508 | 1-{4-[1-(1-benzylpiperidin-4-yl)-4-morpholin-4-yl-1H-pyrazolo[3,4- |
| | d]pyrimidin-6-yl]phenyl}-3-(cyclopropylmethyl)urea |
| 509 | 1-allyl-3-{4-[1-(1-benzylpiperidin-4-yl)-4-morpholin-4-yl-1H- |
| | pyrazolo[3,4-d]pyrimidin-6-yl]phenyl}urea |
| 510 | 1-{4-[1-(1-benzylpiperidin-4-yl)-4-morpholin-4-yl-1H-pyrazolo[3,4- |
| | d]pyrimidin-6-yl]phenyl}-3-(2-hydroxyethyl)urea |
| 511 | 1-{4-[1-(1-benzylpiperidin-4-yl)-4-morpholin-4-yl-1H-pyrazolo[3,4- |
| | d]pyrimidin-6-yl]phenyl}-3-(2-methoxyethyl)urea |
| 512 | 1-(2-aminoethyl)-3-{4-[1-(1-benzylpiperidin-4-yl)-4-morpholin-4-yl- |
| | 1H-pyrazolo[3,4-d]pyrimidin-6-yl]phenyl}urea |
| 513 | 1-{4-[1-(1-benzylpiperidin-4-yl)-4-morpholin-4-yl-1H-pyrazolo[3,4- |
| | d]pyrimidin-6-yl]phenyl}-3-[2-(dimethylamino)ethyl]urea |
| 514 | 1-{4-[1-(1-benzylpiperidin-4-yl)-4-morpholin-4-yl-1H-pyrazolo[3,4- |
| | d]pyrimidin-6-yl]phenyl}-3-(3-hydroxypropyl)urea |
| 515 | 1-{4-[1-(1-benzylpiperidin-4-yl)-4-morpholin-4-yl-1H-pyrazolo[3,4-d]pyrimidin- |
| | 6-yl]phenyl}-3-(3-methoxypropyl)urea |
| 516 | 1-{4-[1-(1-benzylpiperidin-4-yl)-4-morpholin-4-yl-1H-pyrazolo[3,4- |
| | d]pyrimidin-6-yl]phenyl}-3-[3-(dimethylamino)propyl]urea |
| 517 | 1-{4-[1-(1-benzylpiperidin-4-yl)-4-morpholin-4-yl-1H-pyrazolo[3,4- |
| | d]pyrimidin-6-yl]phenyl}-3-(1-methylpiperidin-4-yl)urea |
| 518 | 4-[6-(1H-indol-5-yl)-4-morpholin-4-yl-1H-pyrazolo[3,4-d]pyrimidin- |
| | 1-yl]piperidine-1-carbaldehyde |
| 519 | 3-methoxy-N-{4-[4-morpholin-4-yl-1-(tetrahydro-2H-pyran-2-yl)-1H- |
| | pyrazolo[3,4-d]pyrimidin-6-yl]phenyl}benzamide |
| 520 | methyl {4-[4-morpholin-4-yl-1-(tetrahydro-2H-pyran-2-yl)-1H- |
| | pyrazolo[3,4-d]pyrimidin-6-yl]phenyl}carbamate |
| 521 | N²,N²-dimethyl-N-{4-[4-morpholin-4-yl-1-(tetrahydro-2H-pyran-2-yl)- |
| | 1H-pyrazolo[3,4-d]pyrimidin-6-yl]phenyl}glycinamide |
| 522 | 2-[4-(dimethylamino)phenyl]-N-{4-[4-morpholin-4-yl-1-(tetrahydro- |
| | 2H-pyran-2-yl)-1H-pyrazolo[3,4-d]pyrimidin-6-yl]phenyl}acetamide |
| 523 | 3-methoxy-N-[4-(4-morpholin-4-yl-1H-pyrazolo[3,4-d]pyrimidin-6- |
| | yl)phenyl]benzamide |
| 524 | N-[4-(4-morpholin-4-yl-1H-pyrazolo[3,4-d]pyrimidin-6- |
| | yl)phenyl]nicotinamide |
| 525 | methyl [4-(4-morpholin-4-yl-1H-pyrazolo[3,4-d]pyrimidin-6- |
| | yl)phenyl]carbamate |
| 526 | N-[4-(4-morpholin-4-yl-1H-pyrazolo[3,4-d]pyrimidin-6- |
| | yl)phenyl]acrylamide |
| 527 | N²,N²-dimethyl-N-[4-(4-morpholin-4-yl-1H-pyrazolo[3,4-d]pyrimidin- |
| | 6-yl)phenyl]glycinamide |
| 528 | N-[4-(4-morpholin-4-yl-1H-pyrazolo[3,4-d]pyrimidin-6- |
| | yl)phenyl]glycinamide |
| 529 | N-[4-(4-morpholin-4-yl-1H-pyrazolo[3,4-d]pyrimidin-6-yl)phenyl]-b- |
| | alaninamide |
| 530 | 1-methyl-N-[4-(4-morpholin-4-yl-1H-pyrazolo[3,4-d]pyrimidin-6- |
| | yl)phenyl]piperidine-4-carboxamide |
| 531 | 4-(4-morpholin-4-yl-1H-pyrazolo[3,4-d]pyrimidin-6-yl)aniline |
| 532 | 1-{4-[1-(1-benzylpiperidin-4-yl)-4-morpholin-4-yl-1H-pyrazolo[3,4- |
| | d]pyrimidin-6-yl]phenyl}-3-methoxyurea |
| 533 | 1-{4-[1-(1-benzylpiperidin-4-yl)-4-morpholin-4-yl-1H-pyrazolo[3,4-d]pyrimidin- |
| | 6-yl]phenyl}-3-ethoxyurea |
| 534 | 1-{4-[1-(1-benzylpiperidin-4-yl)-4-morpholin-4-yl-1H-pyrazolo[3,4- |
| | d]pyrimidin-6-yl]phenyl}-3-(2-fluoroethyl)urea |
| 535 | 1-{4-[1-(1-benzylpiperidin-4-yl)-4-morpholin-4-yl-1H-pyrazolo[3,4- |
| | d]pyrimidin-6-yl]phenyl}-3-(2,2,2-trifluoroethyl)urea |
| 536 | N-{4-[1-(1-benzylpiperidin-4-yl)-4-morpholin-4-yl-1H-pyrazolo[3,4- |
| | d]pyrimidin-6-yl]phenyl}-2,2-dimethylhydrazinecarboxamide |
| 537 | 1-{4-[1-(1-benzylpiperidin-4-yl)-4-morpholin-4-yl-1H-pyrazolo[3,4- |
| | d]pyrimidin-6-yl]phenyl}-3-pyrrolidin-1-ylurea |
| 538 | N-[4-(4-morpholin-4-yl-1-phenyl-1H-pyrazolo[3,4-d]pyrimidin-6- |
| | yl)phenyl]hydrazinecarboxamide |
| 539 | 1-hydroxy-3-[4-(4-morpholin-4-yl-1-phenyl-1H-pyrazolo[3,4- |
| | d]pyrimidin-6-yl)phenyl]urea |
| 540 | 1-(2-fluoroethyl)-3-[4-(4-morpholin-4-yl-1-phenyl-1H-pyrazolo[3,4- |
| | d]pyrimidin-6-yl)phenyl]urea |
| 541 | 1-methoxy-3-[4-(4-morpholin-4-yl-1-phenyl-1H-pyrazolo[3,4- |
| | d]pyrimidin-6-yl)phenyl]urea |
| 542 | 1-(allyloxy)-3-[4-(4-morpholin-4-yl-1-phenyl-1H-pyrazolo[3,4- |
| | d]pyrimidin-6-yl)phenyl]urea |
| 543 | 1-methyl-3-[4-(4-morpholin-4-yl-1-phenyl-1H-pyrazolo[3,4-d]pyrimidin- |
| | 6-yl)phenyl]urea |
| 544 | N-{4-[1-(1-benzylpiperidin-4-yl)-4-morpholin-4-yl-1H-pyrazolo[3,4- |
| | d]pyrimidin-6-yl]phenyl}-2,2,2-trifluoroacetamide |
| 545 | 1-{4-[1-(1-benzylpiperidin-4-yl)-4-morpholin-4-yl-1H-pyrazolo[3,4- |
| | d]pyrimidin-6-yl]phenyl}-3-[2-(methylamino)ethyl]urea |
| 546 | 1-(4-{4-morpholin-4-yl-1-[1-(pyridin-3-ylcarbonyl)piperidin-4-yl]- |
| | 1H-pyrazolo[3,4-d]pyrimidin-6-yl}phenyl)urea |
| 547 | 1-(4-{4-morpholin-4-yl-1-[1-(pyridin-3-ylcarbonyl)piperidin-4-yl]- |
| | 1H-pyrazolo[3,4-d]pyrimidin-6-yl}phenyl)-3-pyridin-3-ylurea |
| 548 | 1-(2-fluoroethyl)-3-(4-{4-morpholin-4-yl-1-[1-(pyridin-3- |
| | ylcarbonyl)piperidin-4-yl]-1H-pyrazolo[3,4-d]pyrimidin-6- |
| | yl}phenyl)urea |
| 549 | 1-(2-hydroxyethyl)-3-(4-{4-morpholin-4-yl-1-[1-(pyridin-3- |
| | ylcarbonyl)piperidin-4-yl]-1H-pyrazolo[3,4-d]pyrimidin-6- |
| | yl}phenyl)urea |
| 550 | 1-hydroxy-3-(4-{4-morpholin-4-yl-1-[1-(pyridin-3- |
| | ylcarbonyl)piperidin-4-yl]-1H-pyrazolo[3,4-d]pyrimidin-6- |
| | yl}phenyl)urea |
| 551 | N-(4-{4-morpholin-4-yl-1-[1-(pyridin-3-ylcarbonyl)piperidin-4-yl]- |
| | 1H-pyrazolo[3,4-d]pyrimidin-6-yl}phenyl)hydrazinecarboxamide |
| 552 | 1-ethyl-3-(4-{4-morpholin-4-yl-1-[1-(pyridin-3-ylcarbonyl)piperidin- |
| | 4-yl]-1H-pyrazolo[3,4-d]pyrimidin-6-yl}phenyl)urea |
| 553 | 1-methoxy-3-(4-{4-morpholin-4-yl-1-[1-(pyridin-3- |
| | ylcarbonyl)piperidin-4-yl]-1H-pyrazolo[3,4-d]pyrimidin-6- |
| | yl}phenyl)urea |
| 554 | N-{4-[1-(1-benzylpiperidin-4-yl)-4-morpholin-4-yl-1H-pyrazolo[3,4- |
| | d]pyrimidin-6-yl]phenyl}hydrazinecarboxamide |
| 555 | 1-{4-[1-(1-benzylpiperidin-4-yl)-4-morpholin-4-yl-1H-pyrazolo[3,4- |
| | d]pyrimidin-6-yl]phenyl}-3-hydroxyurea |
| 556 | 1-{4-[1-(1-benzylpiperidin-4-yl)-4-morpholin-4-yl-1H-pyrazolo[3,4- |
| | d]pyrimidin-6-yl]phenyl}-3-cyclopropylurea |
| 557 | 1-{4-[1-(1-benzylpiperidin-4-yl)-4-morpholin-4-yl-1H-pyrazolo[3,4- |
| | d]pyrimidin-6-yl]phenyl}-3-prop-2-yn-1-ylurea |
| 558 | 1-(4-{4-morpholin-4-yl-1-[1-(pyridin-3-ylmethyl)piperidin-4-yl]-1H- |
| | pyrazolo[3,4-d]pyrimidin-6-yl}phenyl)urea |
| 559 | 1-(4-{4-morpholin-4-yl-1-[1-(pyridin-3-ylmethyl)piperidin-4-yl]-1H- |
| | pyrazolo[3,4-d]pyrimidin-6-yl}phenyl)-3-pyridin-3-ylurea |
| 560 | 1-(2-fluoroethyl)-3-(4-{4-morpholin-4-yl-1-[1-(pyridin-3- |
| | ylmethyl)piperidin-4-yl]-1H-pyrazolo[3,4-d]pyrimidin-6- |
| | yl}phenyl)urea |
| 561 | 1-(2-hydroxyethyl)-3-(4-{4-morpholin-4-yl-1-[1-(pyridin-3- |
| | ylmethyl)piperidin-4-yl]-1H-pyrazolo[3,4-d]pyrimidin-6- |
| | yl}phenyl)urea |
| 562 | 1-hydroxy-3-(4-{4-morpholin-4-yl-1-[1-(pyridin-3-ylmethyl)piperidin- |
| | 4-yl]-1H-pyrazolo[3,4-d]pyrimidin-6-yl}phenyl)urea |
| 563 | 1-ethyl-3-(4-{4-morpholin-4-yl-1-[1-(pyridin-3-ylmethyl)piperidin-4- |
| | yl]-1H-pyrazolo[3,4-d]pyrimidin-6-yl}phenyl)urea |
| 564 | 1-methoxy-3-(4-{4-morpholin-4-yl-1-[1-(pyridin-3- |
| | ylmethyl)piperidin-4-yl]-1H-pyrazolo[3,4-d]pyrimidin-6- |
| | yl}phenyl)urea |
| 565 | 4-[1-(1,4-dioxaspiro[4.5]dec-8-yl)-4-morpholin-4-yl-1H-pyrazolo[3,4-d]pyrimidin- |
| | 6-yl]aniline |
| 566 | 1-{4-[1-(1,4-dioxaspiro[4.5]dec-8-yl)-4-morpholin-4-yl-1H- |
| | pyrazolo[3,4-d]pyrimidin-6-yl]phenyl}-3-methylurea |
| 567 | 1-{4-[1-(1,4-dioxaspiro[4.5]dec-8-yl)-4-morpholin-4-yl-1H- |
| | pyrazolo[3,4-d]pyrimidin-6-yl]phenyl}-3-methylurea |
| 568 | 1-methyl-3-{4-[4-morpholin-4-yl-1-(4-oxocyclohexyl)-1H- |
| | pyrazolo[3,4-d]pyrimidin-6-yl]phenyl}urea |
| 569 | 1-{4-[1-(4-hydroxycyclohexyl)-4-morpholin-4-yl-1H-pyrazolo[3,4- |
| | d]pyrimidin-6-yl]phenyl}-3-methylurea |
| 570 | 1-{4-[1-(4-hydroxycyclohexyl)-4-morpholin-4-yl-1H-pyrazolo[3,4- |
| | d]pyrimidin-6-yl]phenyl}-3-methylurea |
| 571 | 1-{4-[1-(1-benzylpiperidin-4-yl)-4-morpholin-4-yl-1H-pyrazolo[3,4- |
| | d]pyrimidin-6-yl]phenyl}-3-methylthiourea |
| 572 | 1-{4-[1-(1-benzylpiperidin-4-yl)-4-morpholin-4-yl-1H-pyrazolo[3,4- |
| | d]pyrimidin-6-yl]phenyl}-3-(1H-imidazol-2-yl)urea |
| 573 | 5-[1-(1-benzylpiperidin-4-yl)-4-morpholin-4-yl-1H-pyrazolo[3,4- |
| | d]pyrimidin-6-yl]-1,3-dihydro-2H-benzimidazol-2-one |
| 574 | 1-methyl-3-[4-(4-morpholin-4-yl-1-{1-[(1-oxidopyridin-3- |
| | yl)carbonyl]piperidin-4-yl}-1H-pyrazolo[3,4-d]pyrimidin-6- |
| | yl)phenyl]urea |
| 575 | 1-methyl-3-[4-(1-{1-[(2-methylpyridin-3-yl)carbonyl]piperidin-4-yl}- |
| | 4-morpholin-4-yl-1H-pyrazolo[3,4-d]pyrimidin-6-yl)phenyl]urea |
| 576 | 1-[4-(1-{1-[(6-methoxypyridin-3-yl)methyl]piperidin-4-yl}-4- |
| | morpholin-4-yl-1H-pyrazolo[3,4-d]pyrimidin-6-yl)phenyl]-3- |
| | methylurea |
| 577 | 1-methyl-3-(4-{4-morpholin-4-yl-1-[1-(pyridin-2-ylmethyl)piperidin- |
| | 4-yl]-1H-pyrazolo[3,4-d]pyrimidin-6-yl}phenyl)urea |
| 578 | 2-[4-(6-{4-[(methylcarbamoyl)amino]phenyl}-4-morpholin-4-yl-1H- |
| | pyrazolo[3,4-d]pyrimidin-1-yl)piperidin-1-yl]acetamide |
| 579 | 1-methyl-3-(4-{4-morpholin-4-yl-1-[1-(2-oxo-2- |
| | phenylethyl)piperidin-4-yl]-1H-pyrazolo[3,4-d]pyrimidin-6- |
| | yl}phenyl)urea |
| 580 | 1-methyl-3-[4-(1-{1-[(4-methylpiperazin-1-yl)carbonyl]piperidin-4- |
| | yl}-4-morpholin-4-yl-1H-pyrazolo[3,4-d]pyrimidin-6-yl)phenyl]urea |
| 581 | 4-(6-{4-[(methylcarbamoyl)amino]phenyl}-4-morpholin-4-yl-1H- |
| | pyrazolo[3,4-d]pyrimidin-1-yl)-N-pyridin-3-ylpiperidine-1- |
| | carboxamide |
| 582 | 1-{4-[1-(1-benzoylpiperidin-4-yl)-4-morpholin-4-yl-1H-pyrazolo[3,4- |
| | d]pyrimidin-6-yl]phenyl}-3-methylurea |
| 583 | 1-{4-[1-(1-benzoylpiperidin-4-yl)-4-morpholin-4-yl-1H-pyrazolo[3,4- |
| | d]pyrimidin-6-yl]phenyl}-3-methylurea |
| 584 | tert-butyl 4-(6-{4-[(methylcarbamoyl)amino]phenyl}-4-morpholin-4- |
| | yl-1H-pyrazolo[3,4-d]pyrimidin-1-yl)piperidine-1-carboxylate |
| 585 | tert-butyl 4-(6-{4-[(methylcarbamoyl)amino]phenyl}-4-morpholin-4- |
| | yl-1H-pyrazolo[3,4-d]pyrimidin-1-yl)piperidine-1-carboxylate |
| 586 | 1-methyl-3-[4-(4-morpholin-4-yl-1-piperidin-4-yl-1H-pyrazolo[3,4-d]pyrimidin- |
| | 6-yl)phenyl]urea |
| 587 | 1-methyl-3-[4-(4-morpholin-4-yl-1-piperidin-4-yl-1H-pyrazolo[3,4- |
| | d]pyrimidin-6-yl)phenyl]urea |
| 588 | 1-(4-{4-morpholin-4-yl-1-[1-(pyridin-3-ylmethyl)piperidin-4-yl]-1H- |
| | pyrazolo[3,4-d]pyrimidin-6-yl}phenyl)-3-phenylurea |
| 589 | 1-(4-{4-morpholin-4-yl-1-[1-(pyridin-3-ylmethyl)piperidin-4-yl]-1H- |
| | pyrazolo[3,4-d]pyrimidin-6-yl}phenyl)-3-pyridin-4-ylurea |
| 590 | 1-(4-{4-morpholin-4-yl-1-[1-(pyridin-3-ylmethyl)piperidin-4-yl]-1H- |
| | pyrazolo[3,4-d]pyrimidin-6-yl}phenyl)-3-pyridin-2-ylurea |
| 591 | 1-[2-(methylamino)ethyl]-3-(4-{4-morpholin-4-yl-1-[1-(pyridin-3- |
| | ylmethyl)piperidin-4-yl]-1H-pyrazolo[3,4-d]pyrimidin-6- |
| | yl}phenyl)urea |
| 592 | 1-(4-{4-morpholin-4-yl-1-[1-(pyridin-3-ylcarbonyl)piperidin-4-yl]- |
| | 1H-pyrazolo[3,4-d]pyrimidin-6-yl}phenyl)-3-phenylurea |
| 593 | 1-(4-{4-morpholin-4-yl-1-[1-(pyridin-3-ylcarbonyl)piperidin-4-yl]- |
| | 1H-pyrazolo[3,4-d]pyrimidin-6-yl}phenyl)-3-pyridin-4-ylurea |
| 594 | 1-(4-{4-morpholin-4-yl-1-[1-(pyridin-3-ylcarbonyl)piperidin-4-yl]- |
| | 1H-pyrazolo[3,4-d]pyrimidin-6-yl}phenyl)-3-pyridin-2-ylurea |
| 595 | 1-(4-{4-morpholin-4-yl-1-[1-(pyridin-3-ylcarbonyl)piperidin-4-yl]- |
| | 1H-pyrazolo[3,4-d]pyrimidin-6-yl}phenyl)-3-pyridin-2-ylurea |
| 596 | 1-[2-(methylamino)ethyl]-3-(4-{4-morpholin-4-yl-1-[1-(pyridin-3- |
| | ylcarbonyl)piperidin-4-yl]-1H-pyrazolo[3,4-d]pyrimidin-6- |
| | yl}phenyl)urea |
| 597 | 4-[1-(1-benzylpiperidin-4-yl)-4-morpholin-4-yl-1H-pyrazolo[3,4- |
| | d]pyrimidin-6-yl]-2-fluoroaniline |
| 598 | 1-{4-[1-(1-benzylpiperidin-4-yl)-4-morpholin-4-yl-1H-pyrazolo[3,4- |
| | d]pyrimidin-6-yl]-2-fluorophenyl}-3-methylurea |
| 599 | 1-{4-[1-(1-benzylpiperidin-4-yl)-4-morpholin-4-yl-1H-pyrazolo[3,4- |
| | d]pyrimidin-6-yl]-2-fluorophenyl}-3-ethylurea |
| 600 | 1-(2-fluoro-4-{4-morpholin-4-yl-1-[1-(pyridin-3-ylmethyl)piperidin-4- |
| | yl]-1H-pyrazolo[3,4-d]pyrimidin-6-yl}phenyl)-3-methylurea |
| 601 | 1-(2-fluoro-4-{4-morpholin-4-yl-1-[1-(pyridin-3-ylmethyl)piperidin-4- |
| | yl]-1H-pyrazolo[3,4-d]pyrimidin-6-yl}phenyl)-3-phenylurea |
| 602 | 1-(2-fluoro-4-{4-morpholin-4-yl-1-[1-(pyridin-3-ylmethyl)piperidin-4- |
| | yl]-1H-pyrazolo[3,4-d]pyrimidin-6-yl}phenyl)-3-pyridin-4-ylurea |
| 603 | 1-(2-fluoro-4-{4-morpholin-4-yl-1-[1-(pyridin-3-ylmethyl)piperidin-4- |
| | yl]-1H-pyrazolo[3,4-d]pyrimidin-6-yl}phenyl)-3-pyridin-4-ylurea |
| 604 | 1-(4-{1-[1-(2-fluorobenzoyl)piperidin-4-yl]-4-morpholin-4-yl-1H- |
| | pyrazolo[3,4-d]pyrimidin-6-yl}phenyl)-3-methylurea |
| 605 | 1-(4-{1-[1-(2-chlorobenzoyl)piperidin-4-yl]-4-morpholin-4-yl-1H- |
| | pyrazolo[3,4-d]pyrimidin-6-yl}phenyl)-3-methylurea |
| 606 | 1-methyl-3-(4-{1-[1-(2-methylbenzoyl)piperidin-4-yl]-4-morpholin-4- |
| | yl-1H-pyrazolo[3,4-d]pyrimidin-6-yl}phenyl)urea |
| 607 | 1-(4-{1-[1-(3-fluorobenzoyl)piperidin-4-yl]-4-morpholin-4-yl-1H- |
| | pyrazolo[3,4-d]pyrimidin-6-yl}phenyl)-3-methylurea |
| 608 | 1-(4-{1-[1-(3-chlorobenzoyl)piperidin-4-yl]-4-morpholin-4-yl-1H- |
| | pyrazolo[3,4-d]pyrimidin-6-yl}phenyl)-3-methylurea |
| 609 | 1-methyl-3-[4-(4-morpholin-4-yl-1-{1-[3- |
| | (trifluoromethyl)benzoyl]piperidin-4-yl}-1H-pyrazolo[3,4- |
| | d]pyrimidin-6-yl)phenyl]urea |
| 610 | 1-(4-{1-[1-(4-bromobenzoyl)piperidin-4-yl]-4-morpholin-4-yl-1H- |
| | pyrazolo[3,4-d]pyrimidin-6-yl}phenyl)-3-methylurea |
| 611 | 1-(4-{1-[1-(4-fluorobenzoyl)piperidin-4-yl]-4-morpholin-4-yl-1H- |
| | pyrazolo[3,4-d]pyrimidin-6-yl}phenyl)-3-methylurea |
| 612 | 1-(4-{1-[1-(4-chlorobenzoyl)piperidin-4-yl]-4-morpholin-4-yl-1H- |
| | pyrazolo[3,4-d]pyrimidin-6-yl}phenyl)-3-methylurea |
| 613 | 1-(4-{1-[1-(4-methoxybenzoyl)piperidin-4-yl]-4-morpholin-4-yl-1H- |
| | pyrazolo[3,4-d]pyrimidin-6-yl}phenyl)-3-methylurea |
| 614 | 1-(4-{1-[1-(3-methoxybenzoyl)piperidin-4-yl]-4-morpholin-4-yl-1H- |
| | pyrazolo[3,4-d]pyrimidin-6-yl}phenyl)-3-methylurea |
| 615 | 1-(4-{1-[1-(2-methoxybenzoyl)piperidin-4-yl]-4-morpholin-4-yl-1H- |
| | pyrazolo[3,4-d]pyrimidin-6-yl}phenyl)-3-methylurea |
| 616 | 1-methyl-3-(4-{1-[1-(3-methylbenzoyl)piperidin-4-yl]-4-morpholin-4- |
| | yl-1H-pyrazolo[3,4-d]pyrimidin-6-yl}phenyl)urea |
| 617 | 1-methyl-3-(4-{1-[1-(4-methylbenzoyl)piperidin-4-yl]-4-morpholin-4- |
| | yl-1H-pyrazolo[3,4-d]pyrimidin-6-yl}phenyl)urea |
| 618 | 1-(4-{1-[1-(4-cyanobenzoyl)piperidin-4-yl]-4-morpholin-4-yl-1H- |
| | pyrazolo[3,4-d]pyrimidin-6-yl}phenyl)-3-methylurea |
| 619 | 2-{4-[1-(1-benzylpiperidin-4-yl)-4-morpholin-4-yl-1H-pyrazolo[3,4- |
| | d]pyrimidin-6-yl]phenyl}acetamide |
| 620 | 2-{4-[1-(1-benzylpiperidin-4-yl)-4-morpholin-4-yl-1H-pyrazolo[3,4- |
| | d]pyrimidin-6-yl]phenyl}-N-methylacetamide |
| 621 | 1-ethyl-3-(2-fluoro-4-{4-morpholin-4-yl-1-[1-(pyridin-3- |
| | ylmethyl)piperidin-4-yl]-1H-pyrazolo[3,4-d]pyrimidin-6- |
| | yl}phenyl)urea |
| 622 | 1-(2-fluoro-4-{4-morpholin-4-yl-1-[1-(pyridin-3-ylmethyl)piperidin-4- |
| | yl]-1H-pyrazolo[3,4-d]pyrimidin-6-yl}phenyl)-3-pyridin-3-ylurea |
| 623 | 1-(2-fluoroethyl)-3-(2-fluoro-4-{4-morpholin-4-yl-1-[1-(pyridin-3- |
| | ylmethyl)piperidin-4-yl]-1H-pyrazolo[3,4-d]pyrimidin-6- |
| | yl}phenyl)urea |
| 624 | 1-(4-{4-morpholin-4-yl-1-[1-(pyridin-3-ylmethyl)piperidin-4-yl]-1H- |
| | pyrazolo[3,4-d]pyrimidin-6-yl}phenyl)-3-(3-thienyl)urea |
| 625 | 1-(2-furylmethyl)-3-(4-{4-morpholin-4-yl-1-[1-(pyridin-3- |
| | ylmethyl)piperidin-4-yl]-1H-pyrazolo[3,4-d]pyrimidin-6- |
| | yl}phenyl)urea |
| 626 | 1-methyl-3-(4-{4-morpholin-4-yl-1-[1-(pyridin-3-ylmethyl)piperidin- |
| | 4-yl]-1H-pyrazolo[3,4-d]pyrimidin-6-yl}phenyl)thiourea |
| 627 | 1-{4-[1-(1-benzoylpiperidin-4-yl)-4-morpholin-4-yl-1H-pyrazolo[3,4- |
| | d]pyrimidin-6-yl]phenyl}-3-phenylurea |
| 628 | 1-{4-[1-(1-benzoylpiperidin-4-yl)-4-morpholin-4-yl-1H-pyrazolo[3,4- |
| | d]pyrimidin-6-yl]phenyl}-3-pyridin-3-ylurea |
| 629 | 1-{4-[1-(1-benzoylpiperidin-4-yl)-4-morpholin-4-yl-1H-pyrazolo[3,4- |
| | d]pyrimidin-6-yl]phenyl}-3-(2-fluoroethyl)urea |
| 630 | 1-{4-[1-(1-benzoylpiperidin-4-yl)-4-morpholin-4-yl-1H-pyrazolo[3,4- |
| | d]pyrimidin-6-yl]phenyl}-3-ethylurea |
| 631 | 1-{4-[1-(1-benzoylpiperidin-4-yl)-4-morpholin-4-yl-1H-pyrazolo[3,4- |
| | d]pyrimidin-6-yl]phenyl}urea |
| 632 | ethyl {4-[1-(1-benzoylpiperidin-4-yl)-4-morpholin-4-yl-1H- |
| | pyrazolo[3,4-d]pyrimidin-6-yl]phenyl}carbamate |
| 633 | 1-{4-[1-(1-benzoylpiperidin-4-yl)-4-morpholin-4-yl-1H-pyrazolo[3,4- |
| | d]pyrimidin-6-yl]phenyl}-3-pyridin-4-ylurea |
| 634 | 1-{4-[1-(1-benzoylpiperidin-4-yl)-4-morpholin-4-yl-1H-pyrazolo[3,4- |
| | d]pyrimidin-6-yl]phenyl}-3-(2-hydroxyethyl)urea |
| 635 | 1-{4-[1-(1-benzoylpiperidin-4-yl)-4-morpholin-4-yl-1H-pyrazolo[3,4- |
| | d]pyrimidin-6-yl]phenyl}-3-[2-(methylamino)ethyl]urea |
| 636 | 1-[4-(1-{1-[(6-fluoropyridin-3-yl)methyl]piperidin-4-yl}-4-morpholin- |
| | 4-yl-1H-pyrazolo[3,4-d]pyrimidin-6-yl)phenyl]-3-methylurea |
| 637 | 1-[4-(1-{1-[(6-chloropyridin-3-yl)methyl]piperidin-4-yl}-4- |
| | morpholin-4-yl-1H-pyrazolo[3,4-d]pyrimidin-6-yl)phenyl]-3-methylurea |
| 638 | 1-[4-(1-{1-[(6-bromopyridin-3-yl)methyl]piperidin-4-yl}-4- |
| | morpholin-4-yl-1H-pyrazolo[3,4-d]pyrimidin-6-yl)phenyl]-3-methylurea |
| 639 | 1-[4-(1-{1-[(2-chloropyridin-3-yl)methyl]piperidin-4-yl}-4- |
| | morpholin-4-yl-1H-pyrazolo[3,4-d]pyrimidin-6-yl)phenyl]-3- |
| | methylurea |
| 640 | 1-[4-(1-{1-[(4-methoxypyridin-3-yl)methyl]piperidin-4-yl}-4- |
| | morpholin-4-yl-1H-pyrazolo[3,4-d]pyrimidin-6-yl)phenyl]-3-methylurea |
| 641 | 1-[4-(1-{1-[(5-fluoropyridin-3-yl)methyl]piperidin-4-yl}-4-morpholin- |
| | 4-yl-1H-pyrazolo[3,4-d]pyrimidin-6-yl)phenyl]-3-methylurea |
| 642 | 1-[4-(1-{1-[(5-bromopyridin-3-yl)methyl]piperidin-4-yl}-4- |
| | morpholin-4-yl-1H-pyrazolo[3,4-d]pyrimidin-6-yl)phenyl]-3- |
| | methylurea |
| 643 | 1-(4-{1-[1-(4-chlorobenzyl)piperidin-4-yl]-4-morpholin-4-yl-1H- |
| | pyrazolo[3,4-d]pyrimidin-6-yl}phenyl)-3-methylurea |
| 644 | 1-(4-{1-[1-(4-chlorobenzyl)piperidin-4-yl]-4-morpholin-4-yl-1H- |
| | pyrazolo[3,4-d]pyrimidin-6-yl}phenyl)-3-methylurea |
| 645 | 1-(4-{1-[1-(3-chlorobenzyl)piperidin-4-yl]-4-morpholin-4-yl-1H- |
| | pyrazolo[3,4-d]pyrimidin-6-yl}phenyl)-3-methylurea |
| 646 | 1-(4-{1-[1-(2-chlorobenzyl)piperidin-4-yl]-4-morpholin-4-yl-1H- |
| | pyrazolo[3,4-d]pyrimidin-6-yl}phenyl)-3-methylurea |
| 647 | 1-(4-{1-[1-(3-hydroxybenzyl)piperidin-4-yl]-4-morpholin-4-yl-1H- |
| | pyrazolo[3,4-d]pyrimidin-6-yl}phenyl)-3-methylurea |
| 648 | 1-(4-{1-[1-(3-hydroxy-4-methoxybenzyl)piperidin-4-yl]-4-morpholin- |
| | 4-yl-1H-pyrazolo[3,4-d]pyrimidin-6-yl}phenyl)-3-methylurea |
| 649 | 1-(4-{1-[1-(2-hydroxybenzyl)piperidin-4-yl]-4-morpholin-4-yl-1H- |
| | pyrazolo[3,4-d]pyrimidin-6-yl}phenyl)-3-methylurea |
| 650 | 1-(4-{1-[1-(3-methoxybenzyl)piperidin-4-yl]-4-morpholin-4-yl-1H- |
| | pyrazolo[3,4-d]pyrimidin-6-yl}phenyl)-3-methylurea |
| 651 | 1-methyl-3-{4-[1-(1-methylpiperidin-4-yl)-4-morpholin-4-yl-1H- |
| | pyrazolo[3,4-d]pyrimidin-6-yl]phenyl}urea |
| 652 | 1-(4-{1-[1-(2-furylmethyl)piperidin-4-yl]-4-morpholin-4-yl-1H- |
| | pyrazolo[3,4-d]pyrimidin-6-yl}phenyl)-3-methylurea |
| 653 | 1-(4-{4-morpholin-4-yl-1-[1-(pyridin-3-ylmethyl)piperidin-4-yl]-1H- |
| | pyrazolo[3,4-d]pyrimidin-6-yl}phenyl)-3-(2-thienyl)urea |
| 654 | 1-cyclopropyl-3-(4-{4-morpholin-4-yl-1-[1-(pyridin-3- |
| | ylmethyl)piperidin-4-yl]-1H-pyrazolo[3,4-d]pyrimidin-6-yl}phenyl)urea |
| 655 | 2-cyano-1-(4-{4-morpholin-4-yl-1-[1-(pyridin-3-ylmethyl)piperidin-4- |
| | yl]-1H-pyrazolo[3,4-d]pyrimidin-6-yl}phenyl)guanidine |
| 656 | 2-cyano-1-methyl-3-(4-{4-morpholin-4-yl-1-[1-(pyridin-3- |
| | ylmethyl)piperidin-4-yl]-1H-pyrazolo[3,4-d]pyrimidin-6- |
| | yl}phenyl)guanidine |
| 657 | 2-cyano-1-ethyl-3-(4-{4-morpholin-4-yl-1-[1-(pyridin-3- |
| | ylmethyl)piperidin-4-yl]-1H-pyrazolo[3,4-d]pyrimidin-6- |
| | yl}phenyl)guanidine |
| 658 | N′-cyano-N-(4-{4-morpholin-4-yl-1-[1-(pyridin-3-ylmethyl)piperidin- |
| | 4-yl]-1H-pyrazolo[3,4-d]pyrimidin-6-yl}phenyl)carbamimidic acid |
| 659 | methyl N′-cyano-N-(4-{4-morpholin-4-yl-1-[1-(pyridin-3- |
| | ylmethyl)piperidin-4-yl]-1H-pyrazolo[3,4-d]pyrimidin-6- |
| | yl}phenyl)imidocarbamate |
| 660 | 2-cyano-1-(4-{4-morpholin-4-yl-1-[1-(pyridin-3-ylcarbonyl)piperidin- |
| | 4-yl]-1H-pyrazolo[3,4-d]pyrimidin-6-yl}phenyl)guanidine |
| 661 | 2-cyano-1-methyl-3-(4-{4-morpholin-4-yl-1-[1-(pyridin-3- |
| | ylcarbonyl)piperidin-4-yl]-1H-pyrazolo[3,4-d]pyrimidin-6- |
| | yl}phenyl)guanidine |
| 662 | 1-(4-{4-morpholin-4-yl-1-[1-(pyridin-3-ylcarbonyl)piperidin-4-yl]- |
| | 1H-pyrazolo[3,4-d]pyrimidin-6-yl}phenyl)-3-(2-thienyl)urea |
| 663 | 1-(4-{4-morpholin-4-yl-1-[1-(pyridin-3-ylcarbonyl)piperidin-4-yl]- |
| | 1H-pyrazolo[3,4-d]pyrimidin-6-yl}phenyl)-3-(3-thienyl)urea |
| 664 | 1-cyclopropyl-3-(4-{4-morpholin-4-yl-1-[1-(pyridin-3- |
| | ylcarbonyl)piperidin-4-yl]-1H-pyrazolo[3,4-d]pyrimidin-6- |
| | yl}phenyl)urea |
| 665 | 6-(2,3-dihydro-1H-indol-5-yl)-4-morpholin-4-yl-1-[1-(pyridin-3- |
| | ylmethyl)piperidin-4-yl]-1H-pyrazolo[3,4-d]pyrimidine |
| 666 | 1-{4-[1-(1-isonicotinoylpiperidin-4-yl)-4-morpholin-4-yl-1H- |
| | pyrazolo[3,4-d]pyrimidin-6-yl]phenyl}-3-methylurea |
| 667 | 1-methyl-3-(4-{4-morpholin-4-yl-1-[1-(pyridin-2- |
| | ylcarbonyl)piperidin-4-yl]-1H-pyrazolo[3,4-d]pyrimidin-6- |
| | yl}phenyl)urea |
| 668 | 1-methyl-3-[4-(1-{1-[(4-methylpyridin-3-yl)carbonyl]piperidin-4-yl}- |
| | 4-morpholin-4-yl-1H-pyrazolo[3,4-d]pyrimidin-6-yl)phenyl]urea |
| 669 | 1-methyl-3-[4-(1-{1-[(6-methylpyridin-3-yl)carbonyl]piperidin-4-yl}- |
| | 4-morpholin-4-yl-1H-pyrazolo[3,4-d]pyrimidin-6-yl)phenyl]urea |
| 670 | 1-[4-(1-{1-[(6-fluoropyridin-3-yl)carbonyl]piperidin-4-yl}-4- |
| | morpholin-4-yl-1H-pyrazolo[3,4-d]pyrimidin-6-yl)phenyl]-3- |
| | methylurea |
| 671 | 1-methyl-3-(4-{4-morpholin-4-yl-1-[1-(pyrazin-2- |
| | ylcarbonyl)piperidin-4-yl]-1H-pyrazolo[3,4-d]pyrimidin-6- |
| | yl}phenyl)urea |
| 672 | 1-(4-{1-[1-(3-acetylbenzoyl)piperidin-4-yl]-4-morpholin-4-yl-1H- |
| | pyrazolo[3,4-d]pyrimidin-6-yl}phenyl)-3-methylurea |
| 673 | 1-[4-(1-{1-[(6-chloropyridin-3-yl)carbonyl]piperidin-4-yl}-4- |
| | morpholin-4-yl-1H-pyrazolo[3,4-d]pyrimidin-6-yl)phenyl]-3- |
| | methylurea |
| 674 | 1-[4-(1-{1-[(2-chloropyridin-3-yl)carbonyl]piperidin-4-yl}-4- |
| | morpholin-4-yl-1H-pyrazolo[3,4-d]pyrimidin-6-yl)phenyl]-3- |
| | methylurea |
| 675 | 1-methyl-3-(4-{4-morpholin-4-yl-1-[1-(pyridin-4-ylmethyl)piperidin- |
| | 4-yl]-1H-pyrazolo[3,4-d]pyrimidin-6-yl}phenyl)urea |
| 676 | 1-(4-{1-[1-(4-fluorobenzyl)piperidin-4-yl]-4-morpholin-4-yl-1H- |
| | pyrazolo[3,4-d]pyrimidin-6-yl}phenyl)-3-methylurea |
| 677 | 1-(4-{1-[1-(3-fluorobenzyl)piperidin-4-yl]-4-morpholin-4-yl-1H- |
| | pyrazolo[3,4-d]pyrimidin-6-yl}phenyl)-3-methylurea |
| 678 | 1-methyl-3-(4-{1-[1-(2-methylbenzyl)piperidin-4-yl]-4-morpholin-4- |
| | yl-1H-pyrazolo[3,4-d]pyrimidin-6-yl}phenyl)urea |
| 679 | 1-methyl-3-(4-{1-[1-(3-methylbenzyl)piperidin-4-yl]-4-morpholin-4- |
| | yl-1H-pyrazolo[3,4-d]pyrimidin-6-yl}phenyl)urea |
| 680 | 1-methyl-3-(4-{1-[1-(4-methylbenzyl)piperidin-4-yl]-4-morpholin-4- |
| | yl-1H-pyrazolo[3,4-d]pyrimidin-6-yl}phenyl)urea |
| 681 | 6-(1H-indazol-5-yl)-4-morpholin-4-yl-1-phenyl-1H-pyrazolo[3,4- |
| | d]pyrimidine |
| 682 | 5-(4-morpholin-4-yl-1-phenyl-1H-pyrazolo[3,4-d]pyrimidin-6-yl)-1,3- |
| | dihydro-2H-indol-2-one |
| 683 | 2-{4-morpholin-4-yl-1-[1-(pyridin-3-ylcarbonyl)piperidin-4-yl]-1H- |
| | pyrazolo[3,4-d]pyrimidin-6-yl}pyridin-4-amine |
| 684 | 6-{4-morpholin-4-yl-1-[1-(pyridin-3-ylcarbonyl)piperidin-4-yl]-1H- |
| | pyrazolo[3,4-d]pyrimidin-6-yl}pyridin-3-amine |
| 685 | 6-{4-morpholin-4-yl-1-[1-(pyridin-3-ylcarbonyl)piperidin-4-yl]-1H- |
| | pyrazolo[3,4-d]pyrimidin-6-yl}pyridin-2-amine |
| 686 | 2-(4-morpholin-4-yl-1-phenyl-1H-pyrazolo[3,4-d]pyrimidin-6- |
| | yl)pyridin-4-amine |
| 687 | 6-(4-morpholin-4-yl-1-phenyl-1H-pyrazolo[3,4-d]pyrimidin-6- |
| | yl)pyridin-3-amine |
| 688 | 6-(4-morpholin-4-yl-1-phenyl-1H-pyrazolo[3,4-d]pyrimidin-6- |
| | yl)pyridin-2-amine |
| 689 | methyl [4-(1-{1-[(4-methylpyridin-3-yl)carbonyl]piperidin-4-yl}-4- |
| | morpholin-4-yl-1H-pyrazolo[3,4-d]pyrimidin-6-yl)phenyl]carbamate |
| 690 | methyl (4-{4-morpholin-4-yl-1-[1-(pyridin-2-ylcarbonyl)piperidin-4- |
| | yl]-1H-pyrazolo[3,4-d]pyrimidin-6-yl}phenyl)carbamate |
| 691 | methyl {4-[1-(1-benzoylpiperidin-4-yl)-4-morpholin-4-yl-1H- |
| | pyrazolo[3,4-d]pyrimidin-6-yl]phenyl}carbamate |
| 692 | methyl (4-{1-[1-(2-fluorobenzoyl)piperidin-4-yl]-4-morpholin-4-yl- |
| | 1H-pyrazolo[3,4-d]pyrimidin-6-yl}phenyl)carbamate |
| 693 | methyl (4-{1-[1-(2-chlorobenzoyl)piperidin-4-yl]-4-morpholin-4-yl- |
| | 1H-pyrazolo[3,4-d]pyrimidin-6-yl}phenyl)carbamate |
| 694 | methyl (4-{1-[1-(4-fluorobenzoyl)piperidin-4-yl]-4-morpholin-4-yl- |
| | 1H-pyrazolo[3,4-d]pyrimidin-6-yl}phenyl)carbamate |
| 695 | methyl (4-{1-[1-(2-methoxybenzoyl)piperidin-4-yl]-4-morpholin-4-yl- |
| | 1H-pyrazolo[3,4-d]pyrimidin-6-yl}phenyl)carbamate |
| 696 | methyl (4-{1-[1-(4-cyanobenzoyl)piperidin-4-yl]-4-morpholin-4-yl- |
| | 1H-pyrazolo[3,4-d]pyrimidin-6-yl}phenyl)carbamate |
| 697 | methyl [4-(1-{1-[(4-methylpiperazin-1-yl)carbonyl]piperidin-4-yl}-4- |
| | morpholin-4-yl-1H-pyrazolo[3,4-d]pyrimidin-6-yl)phenyl]carbamate |
| 698 | methyl (4-{1-[1-(2,4-difluorobenzoyl)piperidin-4-yl]-4-morpholin-4- |
| | yl-1H-pyrazolo[3,4-d]pyrimidin-6-yl}phenyl)carbamate |
| 699 | methyl (4-{1-[1-(4-fluorobenzyl)piperidin-4-yl]-4-morpholin-4-yl-1H- |
| | pyrazolo[3,4-d]pyrimidin-6-yl}phenyl)carbamate |
| 700 | methyl (4-{1-[1-(4-chlorobenzyl)piperidin-4-yl]-4-morpholin-4-yl-1H- |
| | pyrazolo[3,4-d]pyrimidin-6-yl}phenyl)carbamate |
| 701 | methyl [4-(1-{1-[(2-methoxypyridin-3-yl)methyl]piperidin-4-yl}-4- |
| | morpholin-4-yl-1H-pyrazolo[3,4-d]pyrimidin-6-yl)phenyl]carbamate |
| 702 | methyl [4-(1-{1-[(6-fluoropyridin-3-yl)methyl]piperidin-4-yl}-4- |
| | morpholin-4-yl-1H-pyrazolo[3,4-d]pyrimidin-6-yl)phenyl]carbamate |
| 703 | methyl [4-(1-{1-[(6-chloropyridin-3-yl)methyl]piperidin-4-yl}-4- |
| | morpholin-4-yl-1H-pyrazolo[3,4-d]pyrimidin-6-yl)phenyl]carbamate |
| 704 | methyl (4-{1-[1-(2-chlorobenzyl)piperidin-4-yl]-4-morpholin-4-yl-1H- |
| | pyrazolo[3,4-d]pyrimidin-6-yl}phenyl)carbamate |
| 705 | methyl (4-{1-[1-(2-amino-2-oxoethyl)piperidin-4-yl]-4-morpholin-4- |
| | yl-1H-pyrazolo[3,4-d]pyrimidin-6-yl}phenyl)carbamate |
| 706 | methyl (4-{4-morpholin-4-yl-1-[1-(2-oxo-2-phenylethyl)piperidin-4- |
| | yl]-1H-pyrazolo[3,4-d]pyrimidin-6-yl}phenyl)carbamate |
| 707 | methyl {4-[1-(1-acryloylpiperidin-4-yl)-4-morpholin-4-yl-1H- |
| | pyrazolo[3,4-d]pyrimidin-6-yl]phenyl}carbamate |
| 708 | methyl [4-(4-morpholin-4-yl-1-piperidin-4-yl-1H-pyrazolo[3,4- |
| | d]pyrimidin-6-yl)phenyl]carbamate |
| 709 | 3-fluoro-4-{4-morpholin-4-yl-1-[1-(pyridin-3-ylcarbonyl)piperidin-4- |
| | yl]-1H-pyrazolo[3,4-d]pyrimidin-6-yl}aniline |
| 710 | 1-(3-fluoro-4-{4-morpholin-4-yl-1-[1-(pyridin-3-ylcarbonyl)piperidin- |
| | 4-yl]-1H-pyrazolo[3,4-d]pyrimidin-6-yl}phenyl)-3-methylurea |
| 711 | 1-ethyl-3-(3-fluoro-4-{4-morpholin-4-yl-1-[1-(pyridin-3- |
| | ylcarbonyl)piperidin-4-yl]-1H-pyrazolo[3,4-d]pyrimidin-6- |
| | yl}phenyl)urea |
| 712 | 1-(2-fluoroethyl)-3-(3-fluoro-4-{4-morpholin-4-yl-1-[1-(pyridin-3- |
| | ylcarbonyl)piperidin-4-yl]-1H-pyrazolo[3,4-d]pyrimidin-6- |
| | yl}phenyl)urea |
| 713 | 2,5-difluoro-4-{4-morpholin-4-yl-1-[1-(pyridin-3- |
| | ylcarbonyl)piperidin-4-yl]-1H-pyrazolo[3,4-d]pyrimidin-6-yl}aniline |
| 714 | 1-(2,5-difluoro-4-{4-morpholin-4-yl-1-[1-(pyridin-3- |
| | ylcarbonyl)piperidin-4-yl]-1H-pyrazolo[3,4-d]pyrimidin-6-yl}phenyl)- |
| | 3-methylurea |
| 715 | 1-(2,5-difluoro-4-{4-morpholin-4-yl-1-[1-(pyridin-3- |
| | ylcarbonyl)piperidin-4-yl]-1H-pyrazolo[3,4-d]pyrimidin-6-yl}phenyl)- |
| | 3-ethylurea |
| 716 | 1-(2,5-difluoro-4-{4-morpholin-4-yl-1-[1-(pyridin-3- |
| | ylcarbonyl)piperidin-4-yl]-1H-pyrazolo[3,4-d]pyrimidin-6-yl}phenyl)- |
| | 3-(2-fluoroethyl)urea |
| 717 | 1-cyclopropyl-3-(2,5-difluoro-4-{4-morpholin-4-yl-1-[1-(pyridin-3- |
| | ylcarbonyl)piperidin-4-yl]-1H-pyrazolo[3,4-d]pyrimidin-6- |
| | yl}phenyl)urea |
| 718 | 1-(2,5-difluoro-4-{4-morpholin-4-yl-1-[1-(pyridin-3- |
| | ylcarbonyl)piperidin-4-yl]-1H-pyrazolo[3,4-d]pyrimidin-6-yl}phenyl)- |
| | 3-phenylurea |
| 719 | 1-methyl-3-(4-{4-morpholin-4-yl-1-[1-(2,2,2-trifluoroethyl)piperidin- |
| | 4-yl]-1H-pyrazolo[3,4-d]pyrimidin-6-yl}phenyl)urea |
| 720 | 6-(1H-indol-5-yl)-4-morpholin-4-yl-1-[1-(2,2,2- |
| | trifluoroethyl)piperidin-4-yl]-1H-pyrazolo[3,4-d]pyrimidine |
| 721 | 1-{4-[1-(1-acetylpiperidin-4-yl)-4-morpholin-4-yl-1H-pyrazolo[3,4- |
| | d]pyrimidin-6-yl]phenyl}-3-methylurea |
| 722 | N,N-dimethyl-4-(6-{4-[(methylcarbamoyl)amino]phenyl}-4- |
| | morpholin-4-yl-1H-pyrazolo[3,4-d]pyrimidin-1-yl)piperidine-1-carboxamide |
| 723 | 1-(4-{1-[1-(methoxyacetyl)piperidin-4-yl]-4-morpholin-4-yl-1H- |
| | pyrazolo[3,4-d]pyrimidin-6-yl}phenyl)-3-methylurea |
| 724 | 1-{4-[1-(1-isobutyrylpiperidin-4-yl)-4-morpholin-4-yl-1H- |
| | pyrazolo[3,4-d]pyrimidin-6-yl]phenyl}-3-methylurea |
| 725 | methyl 4-(6-{4-[(methylcarbamoyl)amino]phenyl}-4-morpholin-4-yl- |
| | 1H-pyrazolo[3,4-d]pyrimidin-1-yl)piperidine-1-carboxylate |
| 726 | methyl 4-(6-{4-[(methylcarbamoyl)amino]phenyl}-4-morpholin-4-yl- |
| | 1H-pyrazolo[3,4-d]pyrimidin-1-yl)piperidine-1-carboxylate |
| 727 | N-methyl-4-(6-{4-[(methylcarbamoyl)amino]phenyl}-4-morpholin-4- |
| | yl-1H-pyrazolo[3,4-d]pyrimidin-1-yl)piperidine-1-carboxamide |
| 728 | 3-{4-[(2R,6S)-2,6-dimethylmorpholin-4-yl]-1-phenyl-1H- |
| | pyrazolo[3,4-d]pyrimidin-6-yl}phenol |
| 729 | 1-ethyl-3-(5-{4-morpholin-4-yl-1-[1-(pyridin-3-ylmethyl)piperidin-4- |
| | yl]-1H-pyrazolo[3,4-d]pyrimidin-6-yl}pyridin-2-yl)urea |
| 730 | 1-methyl-3-(5-{4-morpholin-4-yl-1-[1-(pyridin-3- |
| | ylcarbonyl)piperidin-4-yl]-1H-pyrazolo[3,4-d]pyrimidin-6-yl}pyridin- |
| | 2-yl)urea |
| 731 | 3-{4-[(3S)-3-methylmorpholin-4-yl]-1-phenyl-1H-pyrazolo[3,4- |
| | d]pyrimidin-6-yl}phenol |
| 732 | 4-[6-(3-hydroxyphenyl)-1-phenyl-1H-pyrazolo[3,4-d]pyrimidin-4- |
| | yl]morpholin-3-one |
| 733 | 3-[4-morpholin-4-yl-1-(2,2,2-trifluoroethyl)-1H-pyrazolo[3,4- |
| | d]pyrimidin-6-yl]phenol |
| 734 | 1-methyl-3-{4-[4-morpholin-4-yl-1-(2,2,2-trifluoroethyl)-1H- |
| | pyrazolo[3,4-d]pyrimidin-6-yl]phenyl}urea |
| 735 | 1-(2-chloro-4-{4-morpholin-4-yl-1-[1-(pyridin-3-ylmethyl)piperidin- |
| | 4-yl]-1H-pyrazolo[3,4-d]pyrimidin-6-yl}phenyl)-3-methylurea |
| 736 | methyl 4-(6-{4-[(ethylcarbamoyl)amino]phenyl}-4-morpholin-4-yl- |
| | 1H-pyrazolo[3,4-d]pyrimidin-1-yl)piperidine-1-carboxylate |
| 737 | methyl 4-[6-(4-{[(2-fluoroethyl)carbamoyl]amino}phenyl)-4- |
| | morpholin-4-yl-1H-pyrazolo[3,4-d]pyrimidin-1-yl]piperidine-1- |
| | carboxylate |
| 738 | methyl 4-[6-(4-{[(2-hydroxyethyl)carbamoyl]amino}phenyl)-4- |
| | morpholin-4-yl-1H-pyrazolo[3,4-d]pyrimidin-1-yl]piperidine-1- |
| | carboxylate |
| 739 | methyl 4-(6-{4-[(cyclopropylcarbamoyl)amino]phenyl}-4-morpholin- |
| | 4-yl-1H-pyrazolo[3,4-d]pyrimidin-1-yl)piperidine-1-carboxylate |
| 740 | methyl 4-(6-{4-[(anilinocarbonyl)amino]phenyl}-4-morpholin-4-yl- |
| | 1H-pyrazolo[3,4-d]pyrimidin-1-yl)piperidine-1-carboxylate |
| 741 | methyl 4-(4-morpholin-4-yl-6-{4-[(pyridin-2- |
| | ylcarbamoyl)amino]phenyl}-1H-pyrazolo[3,4-d]pyrimidin-1- |
| | yl)piperidine-1-carboxylate |
| 742 | methyl 4-(4-morpholin-4-yl-6-{4-[(pyridin-3- |
| | ylcarbamoyl)amino]phenyl}-1H-pyrazolo[3,4-d]pyrimidin-1- |
| | yl)piperidine-1-carboxylate |
| 743 | methyl 4-(4-morpholin-4-yl-6-{4-[(pyridin-4- |
| | ylcarbamoyl)amino]phenyl}-1H-pyrazolo[3,4-d]pyrimidin-1- |
| | yl)piperidine-1-carboxylate |
| 744 | methyl 4-(6-{4-[(methoxycarbonyl)amino]phenyl}-4-morpholin-4-yl- |
| | 1H-pyrazolo[3,4-d]pyrimidin-1-yl)piperidine-1-carboxylate |
| 745 | methyl 4-(6-{4-[(methoxycarbonyl)amino]phenyl}-4-morpholin-4-yl- |
| | 1H-pyrazolo[3,4-d]pyrimidin-1-yl)piperidine-1-carboxylate |
| 746 | methyl 4-[6-(4-aminophenyl)-4-morpholin-4-yl-1H-pyrazolo[3,4- |
| | d]pyrimidin-1-yl]piperidine-1-carboxylate |
| 747 | methyl 4-[6-(4-aminophenyl)-4-morpholin-4-yl-1H-pyrazolo[3,4- |
| | d]pyrimidin-1-yl]piperidine-1-carboxylate |
| 748 | methyl 4-[6-(4-{[(methylamino)carbonothioyl]amino}phenyl)-4- |
| | morpholin-4-yl-1H-pyrazolo[3,4-d]pyrimidin-1-yl]piperidine-1- |
| | carboxylate |
| 749 | 1-(4-{1-[1-(4-hydroxybutyl)piperidin-4-yl]-4-morpholin-4-yl-1H- |
| | pyrazolo[3,4-d]pyrimidin-6-yl}phenyl)-3-methylurea |
| 750 | methyl (4-{1-[1-(4-hydroxybutyl)piperidin-4-yl]-4-morpholin-4-yl- |
| | 1H-pyrazolo[3,4-d]pyrimidin-6-yl}phenyl)carbamate |
| 751 | 1-ethyl-3-(4-{1-[1-(4-hydroxybutyl)piperidin-4-yl]-4-morpholin-4-yl- |
| | 1H-pyrazolo[3,4-d]pyrimidin-6-yl}phenyl)urea |
| 752 | 1-(4-{1-[1-(4-hydroxybutyl)piperidin-4-yl]-4-morpholin-4-yl-1H- |
| | pyrazolo[3,4-d]pyrimidin-6-yl}phenyl)-3-(2-hydroxyethyl)urea |
| 753 | 1-(2-fluoroethyl)-3-(4-{1-[1-(4-hydroxybutyl)piperidin-4-yl]-4- |
| | morpholin-4-yl-1H-pyrazolo[3,4-d]pyrimidin-6-yl}phenyl)urea |
| 754 | 1-(4-{1-[1-(4-hydroxybutyl)piperidin-4-yl]-4-morpholin-4-yl-1H- |
| | pyrazolo[3,4-d]pyrimidin-6-yl}phenyl)-3-phenylurea |
| 755 | 4-{4-[6-(4-aminophenyl)-4-morpholin-4-yl-1H-pyrazolo[3,4- |
| | d]pyrimidin-1-yl]piperidin-1-yl}butan-1-ol |
| 756 | ethyl 4-(6-{4-[(methylcarbamoyl)amino]phenyl}-4-morpholin-4-yl- |
| | 1H-pyrazolo[3,4-d]pyrimidin-1-yl)piperidine-1-carboxylate |
| 757 | propyl 4-(6-{4-[(methylcarbamoyl)amino]phenyl}-4-morpholin-4-yl- |
| | 1H-pyrazolo[3,4-d]pyrimidin-1-yl)piperidine-1-carboxylate |
| 758 | isopropyl 4-(6-{4-[(methylcarbamoyl)amino]phenyl}-4-morpholin-4- |
| | yl-1H-pyrazolo[3,4-d]pyrimidin-1-yl)piperidine-1-carboxylate |
| 759 | vinyl 4-(6-{4-[(methylcarbamoyl)amino]phenyl}-4-morpholin-4-yl- |
| | 1H-pyrazolo[3,4-d]pyrimidin-1-yl)piperidine-1-carboxylate |
| 760 | isobutyl 4-(6-{4-[(methylcarbamoyl)amino]phenyl}-4-morpholin-4-yl- |
| | 1H-pyrazolo[3,4-d]pyrimidin-1-yl)piperidine-1-carboxylate |
| 761 | phenyl 4-(6-{4-[(methylcarbamoyl)amino]phenyl}-4-morpholin-4-yl- |
| | 1H-pyrazolo[3,4-d]pyrimidin-1-yl)piperidine-1-carboxylate |
| 762 | 1-[4-(1-{1-[(2E)-but-2-enoyl]piperidin-4-yl}-4-morpholin-4-yl-1H- |
| | pyrazolo[3,4-d]pyrimidin-6-yl)phenyl]-3-methylurea |
| 763 | methyl 3-[4-(6-{4-[(methylcarbamoyl)amino]phenyl}-4-morpholin-4- |
| | yl-1H-pyrazolo[3,4-d]pyrimidin-1-yl)piperidin-1-yl]-3-oxopropanoate |
| 764 | 1-{4-[1-(1-acryloylpiperidin-4-yl)-4-morpholin-4-yl-1H-pyrazolo[3,4- |
| | d]pyrimidin-6-yl]phenyl}-3-methylurea |
| 765 | 1-methyl-3-(4-{1-[1-(methylsulfonyl)piperidin-4-yl]-4-morpholin-4- |
| | yl-1H-pyrazolo[3,4-d]pyrimidin-6-yl}phenyl)urea |
| 766 | N-methyl-4-(6-{4-[(methylcarbamoyl)amino]phenyl}-4-morpholin-4- |
| | yl-1H-pyrazolo[3,4-d]pyrimidin-1-yl)piperidine-1-carbothioamide |
| 767 | S-methyl 4-(6-{4-[(methylcarbamoyl)amino]phenyl}-4-morpholin-4- |
| | yl-1H-pyrazolo[3,4-d]pyrimidin-1-yl)piperidine-1-carbothioate |
| 768 | S-ethyl 4-(6-{4-[(methylcarbamoyl)amino]phenyl}-4-morpholin-4-yl- |
| | 1H-pyrazolo[3,4-d]pyrimidin-1-yl)piperidine-1-carbothioate |
| 769 | 1-methyl-3-(4-{4-morpholin-4-yl-1-[1-(trifluoroacetyl)piperidin-4-yl]- |
| | 1H-pyrazolo[3,4-d]pyrimidin-6-yl}phenyl)urea |
| 770 | tert-butyl 4-(6-{4-[(ethylcarbamoyl)amino]phenyl}-4-morpholin-4-yl- |
| | 1H-pyrazolo[3,4-d]pyrimidin-1-yl)piperidine-1-carboxylate |
| 771 | tert-butyl 4-[6-(4-{[(2-fluoroethyl)carbamoyl]amino}phenyl)-4- |
| | morpholin-4-yl-1H-pyrazolo[3,4-d]pyrimidin-1-yl]piperidine-1- |
| | carboxylate |
| 772 | tert-butyl 4-[6-(4-{[(2-hydroxyethyl)carbamoyl]amino}phenyl)-4- |
| | morpholin-4-yl-1H-pyrazolo[3,4-d]pyrimidin-1-yl]piperidine-1- |
| | carboxylate |
| 773 | tert-butyl 4-(6-{4-[(cyclopropylcarbamoyl)amino]phenyl}-4- |
| | morpholin-4-yl-1H-pyrazolo[3,4-d]pyrimidin-1-yl)piperidine-1- |
| | carboxylate |
| 774 | tert-butyl 4-(6-{4-[(anilinocarbonyl)amino]phenyl}-4-morpholin-4-yl- |
| | 1H-pyrazolo[3,4-d]pyrimidin-1-yl)piperidine-1-carboxylate |
| 775 | tert-butyl 4-(4-morpholin-4-yl-6-{4-[(pyridin-2- |
| | ylcarbamoyl)amino]phenyl}-1H-pyrazolo[3,4-d]pyrimidin-1- |
| | yl)piperidine-1-carboxylate |
| 776 | tert-butyl 4-(4-morpholin-4-yl-6-{4-[(pyridin-3- |
| | ylcarbamoyl)amino]phenyl}-1H-pyrazolo[3,4-d]pyrimidin-1- |
| | yl)piperidine-1-carboxylate |
| 777 | tert-butyl 4-(4-morpholin-4-yl-6-{4-[(pyridin-4- |
| | ylcarbamoyl)amino]phenyl}-1H-pyrazolo[3,4-d]pyrimidin-1- |
| | yl)piperidine-1-carboxylate |
| 778 | tert-butyl 4-(6-{4-[(methoxycarbonyl)amino]phenyl}-4-morpholin-4- |
| | yl-1H-pyrazolo[3,4-d]pyrimidin-1-yl)piperidine-1-carboxylate |
| 779 | 1-ethyl-3-[4-(4-morpholin-4-yl-1-piperidin-4-yl-1H-pyrazolo[3,4- |
| | d]pyrimidin-6-yl)phenyl]urea |
| 780 | 1-(2-fluoroethyl)-3-[4-(4-morpholin-4-yl-1-piperidin-4-yl-1H- |
| | pyrazolo[3,4-d]pyrimidin-6-yl)phenyl]urea |
| 781 | 1-(2-hydroxyethyl)-3-[4-(4-morpholin-4-yl-1-piperidin-4-yl-1H- |
| | pyrazolo[3,4-d]pyrimidin-6-yl)phenyl]urea |
| 782 | 1-cyclopropyl-3-[4-(4-morpholin-4-yl-1-piperidin-4-yl-1H- |
| | pyrazolo[3,4-d]pyrimidin-6-yl)phenyl]urea |
| 783 | 1-[4-(4-morpholin-4-yl-1-piperidin-4-yl-1H-pyrazolo[3,4-d]pyrimidin- |
| | 6-yl)phenyl]-3-phenylurea |
| 784 | 1-[4-(4-morpholin-4-yl-1-piperidin-4-yl-1H-pyrazolo[3,4-d]pyrimidin- |
| | 6-yl)phenyl]-3-pyridin-2-ylurea |
| 785 | 1-[4-(4-morpholin-4-yl-1-piperidin-4-yl-1H-pyrazolo[3,4-d]pyrimidin- |
| | 6-yl)phenyl]-3-pyridin-3-ylurea |
| 786 | 1-[4-(4-morpholin-4-yl-1-piperidin-4-yl-1H-pyrazolo[3,4-d]pyrimidin- |
| | 6-yl)phenyl]-3-pyridin-4-ylurea |
| 787 | methyl (4-{4-morpholin-4-yl-1-[1-(2,2,2-trifluoroethyl)piperidin-4- |
| | yl]-1H-pyrazolo[3,4-d]pyrimidin-6-yl}phenyl)carbamate |
| 788 | 1-ethyl-3-(4-{4-morpholin-4-yl-1-[1-(2,2,2-trifluoroethyl)piperidin-4- |
| | yl]-1H-pyrazolo[3,4-d]pyrimidin-6-yl}phenyl)urea |
| 789 | 1-(2-fluoroethyl)-3-(4-{4-morpholin-4-yl-1-[1-(2,2,2- |
| | trifluoroethyl)piperidin-4-yl]-1H-pyrazolo[3,4-d]pyrimidin-6- |
| | yl}phenyl)urea |
| 790 | 1-(2-hydroxyethyl)-3-(4-{4-morpholin-4-yl-1-[1-(2,2,2- |
| | trifluoroethyl)piperidin-4-yl]-1H-pyrazolo[3,4-d]pyrimidin-6- |
| | yl}phenyl)urea |
| 791 | 1-(4-{4-morpholin-4-yl-1-[1-(2,2,2-trifluoroethyl)piperidin-4-yl]-1H- |
| | pyrazolo[3,4-d]pyrimidin-6-yl}phenyl)-3-pyridin-2-ylurea |
| 792 | 1-(4-{4-morpholin-4-yl-1-[1-(2,2,2-trifluoroethyl)piperidin-4-yl]-1H- |
| | pyrazolo[3,4-d]pyrimidin-6-yl}phenyl)-3-pyridin-4-ylurea |
| 793 | 1-(4-{4-morpholin-4-yl-1-[1-(2,2,2-trifluoroethyl)piperidin-4-yl]-1H- |
| | pyrazolo[3,4-d]pyrimidin-6-yl}phenyl)-3-pyridin-3-ylurea |
| 794 | 1-cyclopropyl-3-(4-{4-morpholin-4-yl-1-[1-(2,2,2- |
| | trifluoroethyl)piperidin-4-yl]-1H-pyrazolo[3,4-d]pyrimidin-6- |
| | yl}phenyl)urea |
| 795 | 1-(4-{4-morpholin-4-yl-1-[1-(2,2,2-trifluoroethyl)piperidin-4-yl]-1H- |
| | pyrazolo[3,4-d]pyrimidin-6-yl}phenyl)-3-phenylurea |
| 796 | 1-(2-fluoroethyl)-3-{4-[4-morpholin-4-yl-1-(2,2,2-trifluoroethyl)-1H- |
| | pyrazolo[3,4-d]pyrimidin-6-yl]phenyl}urea |
| 797 | 1-(2-hydroxyethyl)-3-{4-[4-morpholin-4-yl-1-(2,2,2-trifluoroethyl)- |
| | 1H-pyrazolo[3,4-d]pyrimidin-6-yl]phenyl}urea |
| 798 | 1-{4-[4-morpholin-4-yl-1-(2,2,2-trifluoroethyl)-1H-pyrazolo[3,4- |
| | d]pyrimidin-6-yl]phenyl}-3-pyridin-3-ylurea |
| 799 | 1-(4-{1-[1-(cyanomethyl)piperidin-4-yl]-4-morpholin-4-yl-1H- |
| | pyrazolo[3,4-d]pyrimidin-6-yl}phenyl)-3-methylurea |
| 800 | methyl [4-(6-{4-[(methylcarbamoyl)amino]phenyl}-4-morpholin-4-yl- |
| | 1H-pyrazolo[3,4-d]pyrimidin-1-yl)piperidin-1-yl]acetate |
| 801 | ethyl [4-(6-{4-[(methylcarbamoyl)amino]phenyl}-4-morpholin-4-yl- |
| | 1H-pyrazolo[3,4-d]pyrimidin-1-yl)piperidin-1-yl]acetate |
| 802 | 1-(4-{1-[1-(methoxymethyl)piperidin-4-yl]-4-morpholin-4-yl-1H- |
| | pyrazolo[3,4-d]pyrimidin-6-yl}phenyl)-3-methylurea |
| 803 | 1-(4-{1-[1-(1,3-dioxolan-2-ylmethyl)piperidin-4-yl]-4-morpholin-4-yl- |
| | 1H-pyrazolo[3,4-d]pyrimidin-6-yl}phenyl)-3-methylurea |
| 804 | [4-(6-{4-[(methylcarbamoyl)amino]phenyl}-4-morpholin-4-yl-1H- |
| | pyrazolo[3,4-d]pyrimidin-1-yl)piperidin-1-yl]acetic acid |
| 805 | 1-{4-[1-(1-allylpiperidin-4-yl)-4-morpholin-4-yl-1H-pyrazolo[3,4- |
| | d]pyrimidin-6-yl]phenyl}-3-methylurea |
| 806 | 2-methoxyethyl 4-(6-{4-[(methylcarbamoyl)amino]phenyl}-4- |
| | morpholin-4-yl-1H-pyrazolo[3,4-d]pyrimidin-1-yl)piperidine-1- |
| | carboxylate |
| 807 | but-2-yn-1-yl 4-(6-{4-[(methylcarbamoyl)amino]phenyl}-4- |
| | morpholin-4-yl-1H-pyrazolo[3,4-d]pyrimidin-1-yl)piperidine-1- |
| | carboxylate |
| 808 | 2-(methylamino)ethyl 4-(6-{4-[(methylcarbamoyl)amino]phenyl}-4- |
| | morpholin-4-yl-1H-pyrazolo[3,4-d]pyrimidin-1-yl)piperidine-1- |
| | carboxylate |
| 809 | 2-(dimethylamino)ethyl 4-(6-{4-[(methylcarbamoyl)amino]phenyl}-4- |
| | morpholin-4-yl-1H-pyrazolo[3,4-d]pyrimidin-1-yl)piperidine-1- |
| | carboxylate |
| 810 | 2-bromoethyl 4-(6-{4-[(methylcarbamoyl)amino]phenyl}-4- |
| | morpholin-4-yl-1H-pyrazolo[3,4-d]pyrimidin-1-yl)piperidine-1- |
| | carboxylate |
| 811 | ethyl 4-(6-{4-[(methoxycarbonyl)amino]phenyl}-4-morpholin-4-yl- |
| | 1H-pyrazolo[3,4-d]pyrimidin-1-yl)piperidine-1-carboxylate |
| 812 | isopropyl 4-(6-{4-[(methoxycarbonyl)amino]phenyl}-4-morpholin-4- |
| | yl-1H-pyrazolo[3,4-d]pyrimidin-1-yl)piperidine-1-carboxylate |
| 813 | S-ethyl 4-(6-{4-[(methoxycarbonyl)amino]phenyl}-4-morpholin-4-yl- |
| | 1H-pyrazolo[3,4-d]pyrimidin-1-yl)piperidine-1-carbothioate |
| 814 | methyl (4-{1-[1-(dimethylcarbamoyl)piperidin-4-yl]-4-morpholin-4- |
| | yl-1H-pyrazolo[3,4-d]pyrimidin-6-yl}phenyl)carbamate |
| 815 | methyl {4-[1-(1-acetylpiperidin-4-yl)-4-morpholin-4-yl-1H- |
| | pyrazolo[3,4-d]pyrimidin-6-yl]phenyl}carbamate |
| 816 | methyl {4-[1-(1-isobutyrylpiperidin-4-yl)-4-morpholin-4-yl-1H- |
| | pyrazolo[3,4-d]pyrimidin-6-yl]phenyl}carbamate |
| 817 | methyl (4-{4-morpholin-4-yl-1-[1-(trifluoroacetyl)piperidin-4-yl]-1H- |
| | pyrazolo[3,4-d]pyrimidin-6-yl}phenyl)carbamate |
| 818 | methyl [4-(1-{1-[(ethylamino)carbonothioyl]piperidin-4-yl}-4- |
| | morpholin-4-yl-1H-pyrazolo[3,4-d]pyrimidin-6-yl)phenyl]carbamate |
| 819 | methyl (4-{1-[1-(methylcarbamoyl)piperidin-4-yl]-4-morpholin-4-yl- |
| | 1H-pyrazolo[3,4-d]pyrimidin-6-yl}phenyl)carbamate |
| 820 | ethyl 4-(6-{4-[(anilinocarbonyl)amino]phenyl}-4-morpholin-4-yl-1H- |
| | pyrazolo[3,4-d]pyrimidin-1-yl)piperidine-1-carboxylate |
| 821 | ethyl 4-(4-morpholin-4-yl-6-{4-[(pyridin-2- |
| | ylcarbamoyl)amino]phenyl}-1H-pyrazolo[3,4-d]pyrimidin-1- |
| | yl)piperidine-1-carboxylate |
| 822 | ethyl 4-(4-morpholin-4-yl-6-{4-[(pyridin-3- |
| | ylcarbamoyl)amino]phenyl}-1H-pyrazolo[3,4-d]pyrimidin-1- |
| | yl)piperidine-1-carboxylate |
| 823 | ethyl 4-(4-morpholin-4-yl-6-{4-[(pyridin-4- |
| | ylcarbamoyl)amino]phenyl}-1H-pyrazolo[3,4-d]pyrimidin-1- |
| | yl)piperidine-1-carboxylate |
| 824 | ethyl 4-[6-(4-{[(2-hydroxyethyl)carbamoyl]amino}phenyl)-4- |
| | morpholin-4-yl-1H-pyrazolo[3,4-d]pyrimidin-1-yl]piperidine-1- |
| | carboxylate |
| 825 | ethyl 4-[6-(4-{[(2-fluoroethyl)carbamoyl]amino}phenyl)-4-morpholin- |
| | 4-yl-1H-pyrazolo[3,4-d]pyrimidin-1-yl]piperidine-1-carboxylate |
| 826 | ethyl 4-(6-{4-[(ethylcarbamoyl)amino]phenyl}-4-morpholin-4-yl-1H- |
| | pyrazolo[3,4-d]pyrimidin-1-yl)piperidine-1-carboxylate |
| 827 | ethyl 4-(6-{4-[(cyclopropylcarbamoyl)amino]phenyl}-4-morpholin-4- |
| | yl-1H-pyrazolo[3,4-d]pyrimidin-1-yl)piperidine-1-carboxylate |
| 828 | 1-ethyl-3-{4-[1-(1-isobutyrylpiperidin-4-yl)-4-morpholin-4-yl-1H- |
| | pyrazolo[3,4-d]pyrimidin-6-yl]phenyl}urea |
| 829 | 1-(2-hydroxyethyl)-3-{4-[1-(1-isobutyrylpiperidin-4-yl)-4-morpholin- |
| | 4-yl-1H-pyrazolo[3,4-d]pyrimidin-6-yl]phenyl}urea |
| 830 | 1-cyclopropyl-3-{4-[1-(1-isobutyrylpiperidin-4-yl)-4-morpholin-4-yl- |
| | 1H-pyrazolo[3,4-d]pyrimidin-6-yl]phenyl}urea |
| 831 | 1-(2-fluoroethyl)-3-{4-[1-(1-isobutyrylpiperidin-4-yl)-4-morpholin-4- |
| | yl-1H-pyrazolo[3,4-d]pyrimidin-6-yl]phenyl}urea |
| 832 | 1-{4-[1-(1-isobutyrylpiperidin-4-yl)-4-morpholin-4-yl-1H- |
| | pyrazolo[3,4-d]pyrimidin-6-yl]phenyl}-3-phenylurea |
| 833 | 1-{4-[1-(1-isobutyrylpiperidin-4-yl)-4-morpholin-4-yl-1H- |
| | pyrazolo[3,4-d]pyrimidin-6-yl]phenyl}-3-pyridin-2-ylurea |
| 834 | 1-{4-[1-(1-isobutyrylpiperidin-4-yl)-4-morpholin-4-yl-1H- |
| | pyrazolo[3,4-d]pyrimidin-6-yl]phenyl}-3-pyridin-3-ylurea |
| 835 | 1-{4-[1-(1-isobutyrylpiperidin-4-yl)-4-morpholin-4-yl-1H- |
| | pyrazolo[3,4-d]pyrimidin-6-yl]phenyl}-3-pyridin-4-ylurea |
| 836 | isopropyl 4-[6-(4-aminophenyl)-4-morpholin-4-yl-1H-pyrazolo[3,4- |
| | d]pyrimidin-1-yl]piperidine-1-carboxylate |
| 837 | isopropyl 4-[6-(4-{[(methylamino)carbonothioyl]amino}phenyl)-4- |
| | morpholin-4-yl-1H-pyrazolo[3,4-d]pyrimidin-1-yl]piperidine-1- |
| | carboxylate |
| 838 | isopropyl 4-[6-(4-{[(1E)- |
| | (methylamino)(methylthio)methylene]amino}phenyl)-4-morpholin-4- |
| | yl-1H-pyrazolo[3,4-d]pyrimidin-1-yl]piperidine-1-carboxylate |
| 839 | isopropyl 4-[6-(4-{[(2-fluoroethyl)carbamoyl]amino}phenyl)-4- |
| | morpholin-4-yl-1H-pyrazolo[3,4-d]pyrimidin-1-yl]piperidine-1- |
| | carboxylate |
| 840 | isopropyl 4-[6-(4-{[(2-hydroxyethyl)carbamoyl]amino}phenyl)-4- |
| | morpholin-4-yl-1H-pyrazolo[3,4-d]pyrimidin-1-yl]piperidine-1- |
| | carboxylate |
| 841 | isopropyl 4-(6-{4-[(cyclopropylcarbamoyl)amino]phenyl}-4- |
| | morpholin-4-yl-1H-pyrazolo[3,4-d]pyrimidin-1-yl)piperidine-1- |
| | carboxylate |
| 842 | isopropyl 4-(6-{4-[(anilinocarbonyl)amino]phenyl}-4-morpholin-4-yl- |
| | 1H-pyrazolo[3,4-d]pyrimidin-1-yl)piperidine-1-carboxylate |
| 843 | isopropyl 4-(4-morpholin-4-yl-6-{4-[(pyridin-2- |
| | ylcarbamoyl)amino]phenyl}-1H-pyrazolo[3,4-d]pyrimidin-1- |
| | yl)piperidine-1-carboxylate |
| 844 | isopropyl 4-(4-morpholin-4-yl-6-{4-[(pyridin-3- |
| | ylcarbamoyl)amino]phenyl}-1H-pyrazolo[3,4-d]pyrimidin-1- |
| | yl)piperidine-1-carboxylate |
| 845 | isopropyl 4-(4-morpholin-4-yl-6-{4-[(pyridin-4- |
| | ylcarbamoyl)amino]phenyl}-1H-pyrazolo[3,4-d]pyrimidin-1- |
| | yl)piperidine-1-carboxylate |
| 846 | methyl 4-[6-{4-[(methoxycarbonyl)amino]phenyl}-4-(2- |
| | methylmorpholin-4-yl)-1H-pyrazolo[3,4-d]pyrimidin-1-yl]piperidine- |
| | 1-carboxylate |
| 847 | methyl 4-[6-{4-[(methylcarbamoyl)amino]phenyl}-4-(2- |
| | methylmorpholin-4-yl)-1H-pyrazolo[3,4-d]pyrimidin-1-yl]piperidine- |
| | 1-carboxylate |
| 848 | methyl 4-[6-{4-[(ethylcarbamoyl)amino]phenyl}-4-(2- |
| | methylmorpholin-4-yl)-1H-pyrazolo[3,4-d]pyrimidin-1-yl]piperidine- |
| | 1-carboxylate |
| 849 | methyl 4-[6-{4-[(cyclopropylcarbamoyl)amino]phenyl}-4-(2- |
| | methylmorpholin-4-yl)-1H-pyrazolo[3,4-d]pyrimidin-1-yl]piperidine- |
| | 1-carboxylate |
| 850 | methyl 4-[6-(4-{[(2-fluoroethyl)carbamoyl]amino}phenyl)-4-(2- |
| | methylmorpholin-4-yl)-1H-pyrazolo[3,4-d]pyrimidin-1-yl]piperidine- |
| | 1-carboxylate |
| 851 | methyl 4-[6-(4-{[(2-hydroxyethyl)carbamoyl]amino}phenyl)-4-(2- |
| | methylmorpholin-4-yl)-1H-pyrazolo[3,4-d]pyrimidin-1-yl]piperidine- |
| | 1-carboxylate |
| 852 | methyl 4-[4-(2-methylmorpholin-4-yl)-6-{4-[(pyridin-3- |
| | ylcarbamoyl)amino]phenyl}-1H-pyrazolo[3,4-d]pyrimidin-1- |
| | yl]piperidine-1-carboxylate |
| 853 | methyl 4-[4-(2-methylmorpholin-4-yl)-6-{4-[(pyridin-2- |
| | ylcarbamoyl)amino]phenyl}-1H-pyrazolo[3,4-d]pyrimidin-1- |
| | yl]piperidine-1-carboxylate |
| 854 | methyl 4-[6-{4-[(anilinocarbonyl)amino]phenyl}-4-(2- |
| | methylmorpholin-4-yl)-1H-pyrazolo[3,4-d]pyrimidin-1-yl]piperidine- |
| | 1-carboxylate |
| 855 | propyl 4-(4-morpholin-4-yl-6-{4-[(phenylcarbamoyl)amino]phenyl}- |
| | 1H-pyrazolo[3,4-d]pyrimidin-1-yl)piperidine-1-carboxylate |
| 856 | propyl 4-(4-morpholin-4-yl-6-{4-[(pyridin-3- |
| | ylcarbamoyl)amino]phenyl}-1H-pyrazolo[3,4-d]pyrimidin-1- |
| | yl)piperidine-1-carboxylate |
| 857 | propyl 4-(4-morpholin-4-yl-6-{4-[(pyridin-4- |
| | ylcarbamoyl)amino]phenyl}-1H-pyrazolo[3,4-d]pyrimidin-1- |
| | yl)piperidine-1-carboxylate |
| 858 | propyl 4-(6-{4-[(ethylcarbamoyl)amino]phenyl}-4-morpholin-4-yl- |
| | 1H-pyrazolo[3,4-d]pyrimidin-1-yl)piperidine-1-carboxylate |
| 859 | propyl 4-(4-morpholin-4-yl-6-{4-[(propylcarbamoyl)amino]phenyl}- |
| | 1H-pyrazolo[3,4-d]pyrimidin-1-yl)piperidine-1-carboxylate |
| 860 | propyl 4-[6-(4-{[(2-fluoroethyl)carbamoyl]amino}phenyl)-4- |
| | morpholin-4-yl-1H-pyrazolo[3,4-d]pyrimidin-1-yl]piperidine-1- |
| | carboxylate |
| 861 | propyl 4-[6-(4-{[(2-hydroxyethyl)carbamoyl]amino}phenyl)-4- |
| | morpholin-4-yl-1H-pyrazolo[3,4-d]pyrimidin-1-yl]piperidine-1- |
| | carboxylate |
| 862 | propyl 4-(6-{4-[(cyclopropylcarbamoyl)amino]phenyl}-4-morpholin- |
| | 4-yl-1H-pyrazolo[3,4-d]pyrimidin-1-yl)piperidine-1-carboxylate |
| 863 | propyl 4-(6-{4-[(methoxycarbonyl)amino]phenyl}-4-morpholin-4-yl- |
| | 1H-pyrazolo[3,4-d]pyrimidin-1-yl)piperidine-1-carboxylate |
| 864 | propyl 4-[6-(4-{[(cyclopropylmethyl)carbamoyl]amino}phenyl)-4- |
| | morpholin-4-yl-1H-pyrazolo[3,4-d]pyrimidin-1-yl]piperidine-1- |
| | carboxylate |
| 865 | methyl 4-[6-(4-{[(4-fluorophenyl)carbamoyl]amino}phenyl)-4- |
| | morpholin-4-yl-1H-pyrazolo[3,4-d]pyrimidin-1-yl]piperidine-1- |
| | carboxylate |
| 866 | methyl 4-[6-(4-{[(2-fluorophenyl)carbamoyl]amino}phenyl)-4- |
| | morpholin-4-yl-1H-pyrazolo[3,4-d]pyrimidin-1-yl]piperidine-1- |
| | carboxylate |
| 867 | methyl 4-[6-(4-{[(2,4-difluorophenyl)carbamoyl]amino}phenyl)-4- |
| | morpholin-4-yl-1H-pyrazolo[3,4-d]pyrimidin-1-yl]piperidine-1- |
| | carboxylate |
| 868 | methyl 4-[6-(4-{[(6-fluoropyridin-3-yl)carbamoyl]amino}phenyl)-4- |
| | morpholin-4-yl-1H-pyrazolo[3,4-d]pyrimidin-1-yl]piperidine-1- |
| | carboxylate |
| 869 | methyl 4-[6-(4-{[(2-fluoropyridin-3-yl)carbamoyl]amino}phenyl)-4- |
| | morpholin-4-yl-1H-pyrazolo[3,4-d]pyrimidin-1-yl]piperidine-1- |
| | carboxylate |
| 870 | methyl 4-[6-(4-{[(3-fluoropyridin-4-yl)carbamoyl]amino}phenyl)-4- |
| | morpholin-4-yl-1H-pyrazolo[3,4-d]pyrimidin-1-yl]piperidine-1- |
| | carboxylate |
| 871 | methyl 4-[6-(4-{[(2-fluoroethoxy)carbonyl]amino}phenyl)-4- |
| | morpholin-4-yl-1H-pyrazolo[3,4-d]pyrimidin-1-yl]piperidine-1- |
| | carboxylate |
| 872 | methyl 4-[6-(4-{[(2-fluorophenoxy)carbonyl]amino}phenyl)-4- |
| | morpholin-4-yl-1H-pyrazolo[3,4-d]pyrimidin-1-yl]piperidine-1- |
| | carboxylate |
| 873 | 1-methyl-3-{4-[4-morpholin-4-yl-1-(tetrahydro-2H-thiopyran-4-yl)- |
| | 1H-pyrazolo[3,4-d]pyrimidin-6-yl]phenyl}urea |
| 874 | 1-methyl-3-{4-[4-morpholin-4-yl-1-(1-oxidotetrahydro-2H-thiopyran- |
| | 4-yl)-1H-pyrazolo[3,4-d]pyrimidin-6-yl]phenyl}urea |
| 875 | 1-{4-[1-(1,1-dioxidotetrahydro-2H-thiopyran-4-yl)-4-morpholin-4-yl- |
| | 1H-pyrazolo[3,4-d]pyrimidin-6-yl]phenyl}-3-methylurea |
| 876 | tert-butyl (3S)-3-[6-(4-aminophenyl)-4-morpholin-4-yl-1H- |
| | pyrazolo[3,4-d]pyrimidin-1-yl]piperidine-1-carboxylate |
| 877 | tert-butyl (3R)-3-[6-(4-aminophenyl)-4-morpholin-4-yl-1H- |
| | pyrazolo[3,4-d]pyrimidin-1-yl]piperidine-1-carboxylate |
| 878 | tert-butyl (3S)-3-(6-{4-[(methylcarbamoyl)amino]phenyl}-4- |
| | morpholin-4-yl-1H-pyrazolo[3,4-d]pyrimidin-1-yl)piperidine-1- |
| | carboxylate |
| 879 | 1-methyl-3-(4-{4-morpholin-4-yl-1-[(3S)-piperidin-3-yl]-1H- |
| | pyrazolo[3,4-d]pyrimidin-6-yl}phenyl)urea |
| 880 | tert-butyl (3R)-3-(6-{4-[(methylcarbamoyl)amino]phenyl}-4- |
| | morpholin-4-yl-1H-pyrazolo[3,4-d]pyrimidin-1-yl)piperidine-1- |
| | carboxylate |
| 881 | 1-methyl-3-(4-{4-morpholin-4-yl-1-[(3R)-piperidin-3-yl]-1H- |
| | pyrazolo[3,4-d]pyrimidin-6-yl}phenyl)urea |
| 882 | 2,2-dimethylpropyl 4-(6-{4-[(methylcarbamoyl)amino]phenyl}-4- |
| | morpholin-4-yl-1H-pyrazolo[3,4-d]pyrimidin-1-yl)piperidine-1- |
| | carboxylate |
| 883 | 2,2-dimethylpropyl 4-(4-morpholin-4-yl-6-{4-[(pyridin-3- |
| | ylcarbamoyl)amino]phenyl}-1H-pyrazolo[3,4-d]pyrimidin-1- |
| | yl)piperidine-1-carboxylate |
| 884 | 2-fluoroethyl 4-(6-{4-[(methylcarbamoyl)amino]phenyl}-4- |
| | morpholin-4-yl-1H-pyrazolo[3,4-d]pyrimidin-1-yl)piperidine-1- |
| | carboxylate |
| 885 | 2-fluoroethyl 4-(4-morpholin-4-yl-6-{4-[(pyridin-3- |
| | ylcarbamoyl)amino]phenyl}-1H-pyrazolo[3,4-d]pyrimidin-1- |
| | yl)piperidine-1-carboxylate |
| 886 | benzyl 4-(6-{4-[(methylcarbamoyl)amino]phenyl}-4-morpholin-4-yl- |
| | 1H-pyrazolo[3,4-d]pyrimidin-1-yl)piperidine-1-carboxylate |
| 887 | benzyl 4-(4-morpholin-4-yl-6-{4-[(pyridin-3- |
| | ylcarbamoyl)amino]phenyl}-1H-pyrazolo[3,4-d]pyrimidin-1- |
| | yl)piperidine-1-carboxylate |
| 888 | tert-butyl 4-(6-{4-[(isoxazol-3-ylcarbamoyl)amino]phenyl}-4- |
| | morpholin-4-yl-1H-pyrazolo[3,4-d]pyrimidin-1-yl)piperidine-1- |
| | carboxylate |
| 889 | tert-butyl 4-[6-(4-{[(3-methylisoxazol-5-yl)carbamoyl]amino}phenyl)- |
| | 4-morpholin-4-yl-1H-pyrazolo[3,4-d]pyrimidin-1-yl]piperidine-1- |
| | carboxylate |
| 890 | tert-butyl 4-(4-morpholin-4-yl-6-{4-[(1,3-thiazol-2- |
| | ylcarbamoyl)amino]phenyl}-1H-pyrazolo[3,4-d]pyrimidin-1- |
| | yl)piperidine-1-carboxylate |
| 891 | tert-butyl 4-(4-morpholin-4-yl-6-{4-[(pyrazin-2- |
| | ylcarbamoyl)amino]phenyl}-1H-pyrazolo[3,4-d]pyrimidin-1- |
| | yl)piperidine-1-carboxylate |
| 892 | tert-butyl 4-(4-morpholin-4-yl-6-{4-[(pyrimidin-2- |
| | ylcarbamoyl)amino]phenyl}-1H-pyrazolo[3,4-d]pyrimidin-1- |
| | yl)piperidine-1-carboxylate |
| 893 | ethyl 4-{6-[4-(1H-imidazol-2-ylamino)phenyl]-4-morpholin-4-yl-1H- |
| | pyrazolo[3,4-d]pyrimidin-1-yl}piperidine-1-carboxylate |
| 894 | ethyl 4-(6-{4-[(methylcarbamoyl)amino]phenyl}-4-[(3R)-3- |
| | methylmorpholin-4-yl]-1H-pyrazolo[3,4-d]pyrimidin-1-yl)piperidine- |
| | 1-carboxylate |
| 895 | ethyl 4-(6-{4-[(ethylcarbamoyl)amino]phenyl}-4-[(3R)-3- |
| | methylmorpholin-4-yl]-1H-pyrazolo[3,4-d]pyrimidin-1-yl)piperidine- |
| | 1-carboxylate |
| 896 | ethyl 4-{6-(4-{[(2-fluoroethyl)carbamoyl]amino}phenyl)-4-[(3R)-3- |
| | methylmorpholin-4-yl]-1H-pyrazolo[3,4-d]pyrimidin-1-yl}piperidine- |
| | 1-carboxylate |
| 897 | ethyl 4-(4-[(3R)-3-methylmorpholin-4-yl]-6-{4- |
| | [(phenylcarbamoyl)amino]phenyl}-1H-pyrazolo[3,4-d]pyrimidin-1- |
| | yl)piperidine-1-carboxylate |
| 898 | ethyl 4-(4-[(3R)-3-methylmorpholin-4-yl]-6-{4-[(pyridin-3- |
| | ylcarbamoyl)amino]phenyl}-1H-pyrazolo[3,4-d]pyrimidin-1- |
| | yl)piperidine-1-carboxylate |
| 899 | ethyl 4-(4-[(3R)-3-methylmorpholin-4-yl]-6-{4-[(pyridin-4- |
| | ylcarbamoyl)amino]phenyl}-1H-pyrazolo[3,4-d]pyrimidin-1- |
| | yl)piperidine-1-carboxylate |
| 900 | ethyl 4-(6-{4-[(ethylcarbamoyl)amino]phenyl}-4-[(3S)-3- |
| | methylmorpholin-4-yl]-1H-pyrazolo[3,4-d]pyrimidin-1-yl)piperidine- |
| | 1-carboxylate |
| 901 | ethyl 4-{6-(4-{[(2-fluoroethyl)carbamoyl]amino}phenyl)-4-[(3S)-3- |
| | methylmorpholin-4-yl]-1H-pyrazolo[3,4-d]pyrimidin-1-yl}piperidine- |
| | 1-carboxylate |
| 902 | ethyl 4-(4-[(3S)-3-methylmorpholin-4-yl]-6-{4- |
| | [(phenylcarbamoyl)amino]phenyl}-1H-pyrazolo[3,4-d]pyrimidin-1- |
| | yl)piperidine-1-carboxylate |
| 903 | ethyl 4-(4-[(3S)-3-methylmorpholin-4-yl]-6-{4-[(pyridin-3- |
| | ylcarbamoyl)amino]phenyl}-1H-pyrazolo[3,4-d]pyrimidin-1- |
| | yl)piperidine-1-carboxylate |
| 904 | ethyl 4-(4-[(3S)-3-methylmorpholin-4-yl]-6-{4-[(pyridin-4- |
| | ylcarbamoyl)amino]phenyl}-1H-pyrazolo[3,4-d]pyrimidin-1- |
| | yl)piperidine-1-carboxylate |
| 905 | ethyl 4-{4-[(3S)-3-methylmorpholin-4-yl]-6-(4-{[(4-morpholin-4- |
| | ylphenyl)carbamoyl]amino}phenyl)-1H-pyrazolo[3,4-d]pyrimidin-1- |
| | yl}piperidine-1-carboxylate |
| 906 | methyl 4-(6-{4-[(ethoxycarbonyl)amino]phenyl}-4-morpholin-4-yl- |
| | 1H-pyrazolo[3,4-d]pyrimidin-1-yl)piperidine-1-carboxylate |
| 907 | methyl 4-[6-(4-{[(2-hydroxyethoxy)carbonyl]amino}phenyl)-4- |
| | morpholin-4-yl-1H-pyrazolo[3,4-d]pyrimidin-1-yl]piperidine-1- |
| | carboxylate |
| 908 | methyl 4-[6-(4-{[(2-methoxyethoxy)carbonyl]amino}phenyl)-4- |
| | morpholin-4-yl-1H-pyrazolo[3,4-d]pyrimidin-1-yl]piperidine-1- |
| | carboxylate |
| 909 | methyl 4-[6-(4-{[(2-aminoethoxy)carbonyl]amino}phenyl)-4- |
| | morpholin-4-yl-1H-pyrazolo[3,4-d]pyrimidin-1-yl]piperidine-1- |
| | carboxylate |
| 910 | methyl 4-{6-[4-({[2-(dimethylamino)ethoxy]carbonyl}amino)phenyl]- |
| | 4-morpholin-4-yl-1H-pyrazolo[3,4-d]pyrimidin-1-yl}piperidine-1- |
| | carboxylate |
| 911 | methyl 4-[4-morpholin-4-yl-6-(4-{[(2-pyrrolidin-1- |
| | ylethoxy)carbonyl]amino}phenyl)-1H-pyrazolo[3,4-d]pyrimidin-1- |
| | yl]piperidine-1-carboxylate |
| 912 | methyl 4-[4-morpholin-4-yl-6-(4-{[(2-morpholin-4- |
| | ylethoxy)carbonyl]amino}phenyl)-1H-pyrazolo[3,4-d]pyrimidin-1- |
| | yl]piperidine-1-carboxylate |
| 913 | methyl 4-{6-[4-({[2-(4-methylpiperazin-1- |
| | yl)ethoxy]carbonyl}amino)phenyl]-4-morpholin-4-yl-1H- |
| | pyrazolo[3,4-d]pyrimidin-1-yl}piperidine-1-carboxylate |
| 914 | methyl 4-[4-morpholin-4-yl-6-(4-{[(2,2,2- |
| | trifluoroethoxy)carbonyl]amino}phenyl)-1H-pyrazolo[3,4- |
| | d]pyrimidin-1-yl]piperidine-1-carboxylate |
| 915 | methyl 4-[6-(4-{[(3-hydroxypropoxy)carbonyl]amino}phenyl)-4- |
| | morpholin-4-yl-1H-pyrazolo[3,4-d]pyrimidin-1-yl]piperidine-1- |
| | carboxylate |
| 916 | methyl 4-{6-[4-({[4-(4-methylpiperazin-1- |
| | yl)phenyl]carbamoyl}amino)phenyl]-4-morpholin-4-yl-1H- |
| | pyrazolo[3,4-d]pyrimidin-1-yl}piperidine-1-carboxylate |
| 917 | methyl 4-[4-morpholin-4-yl-6-(4-{[(6-morpholin-4-ylpyridin-3- |
| | yl)carbamoyl]amino}phenyl)-1H-pyrazolo[3,4-d]pyrimidin-1- |
| | yl]piperidine-1-carboxylate |
| 918 | methyl 4-{6-[4-({[4- |
| | (hydroxymethyl)phenyl]carbamoyl}amino)phenyl]-4-morpholin-4-yl- |
| | 1H-pyrazolo[3,4-d]pyrimidin-1-yl}piperidine-1-carboxylate |
| 919 | methyl 4-{6-[4-({[4-(2- |
| | hydroxyethyl)phenyl]carbamoyl}amino)phenyl]-4-morpholin-4-yl-1H- |
| | pyrazolo[3,4-d]pyrimidin-1-yl}piperidine-1-carboxylate |
| 920 | methyl 4-{4-morpholin-4-yl-6-[4-({[4-(2-pyrrolidin-1- |
| | ylethyl)phenyl]carbamoyl}amino)phenyl]-1H-pyrazolo[3,4- |
| | d]pyrimidin-1-yl}piperidine-1-carboxylate |
| 921 | methyl 4-{4-morpholin-4-yl-6-[4-({[4-(2-piperidin-1- |
| | ylethyl)phenyl]carbamoyl}amino)phenyl]-1H-pyrazolo[3,4- |
| | d]pyrimidin-1-yl}piperidine-1-carboxylate |
| 922 | methyl 4-{4-morpholin-4-yl-6-[4-({[4-(2-piperazin-1- |
| | ylethyl)phenyl]carbamoyl}amino)phenyl]-1H-pyrazolo[3,4- |
| | d]pyrimidin-1-yl}piperidine-1-carboxylate |
| 923 | methyl 4-(6-{4-[({4-[2-(4-methylpiperazin-1- |
| | yl)ethyl]phenyl}carbamoyl)amino]phenyl}-4-morpholin-4-yl-1H- |
| | pyrazolo[3,4-d]pyrimidin-1-yl)piperidine-1-carboxylate |
| 924 | methyl 4-{4-morpholin-4-yl-6-[4-({[4-(2-morpholin-4- |
| | ylethyl)phenyl]carbamoyl}amino)phenyl]-1H-pyrazolo[3,4- |
| | d]pyrimidin-1-yl}piperidine-1-carboxylate |
| 925 | methyl 4-{6-[4-({[4-(2-{[2- |
| | (dimethylamino)ethyl]amino}ethyl)phenyl]carbamoyl}amino)phenyl]- |
| | 4-morpholin-4-yl-1H-pyrazolo[3,4-d]pyrimidin-1-yl}piperidine-1- |
| | carboxylate |
| 926 | methyl 4-[6-(4-{[(4-{2-[(2- |
| | aminoethyl)amino]ethyl}phenyl)carbamoyl]amino}phenyl)-4- |
| | morpholin-4-yl-1H-pyrazolo[3,4-d]pyrimidin-1-yl]piperidine-1- |
| | carboxylate |
| 927 | methyl 4-[6-(4-{[(4-{2-[(2- |
| | hydroxyethyl)amino]ethyl}phenyl)carbamoyl]amino}phenyl)-4- |
| | morpholin-4-yl-1H-pyrazolo[3,4-d]pyrimidin-1-yl]piperidine-1- |
| | carboxylate |
| 928 | methyl 4-[6-(4-{[(4-{2-[(2- |
| | methoxyethyl)amino]ethyl}phenyl)carbamoyl]amino}phenyl)-4- |
| | morpholin-4-yl-1H-pyrazolo[3,4-d]pyrimidin-1-yl]piperidine-1- |
| | carboxylate |
| 929 | 2-hydroxyethyl (4-{4-morpholin-4-yl-1-[1-(2,2,2- |
| | trifluoroethyl)piperidin-4-yl]-1H-pyrazolo[3,4-d]pyrimidin-6- |
| | yl}phenyl)carbamate |
| 930 | 2-hydroxyethyl (4-{4-morpholin-4-yl-1-[1-(pyridin-3- |
| | ylmethyl)piperidin-4-yl]-1H-pyrazolo[3,4-d]pyrimidin-6- |
| | yl}phenyl)carbamate |
| 931 | N-{4-[4-morpholin-4-yl-1-(tetrahydro-2H-pyran-2-yl)-1H- |
| | pyrazolo[3,4-d]pyrimidin-6-yl]phenyl}acetamide |
| 932 | N-[4-(4-morpholin-4-yl-1H-pyrazolo[3,4-d]pyrimidin-6- |
| | yl)phenyl]acetamide |
| 933 | 1-methyl-3-[4-(4-morpholin-4-yl-1H-pyrazolo[3,4-d]pyrimidin-6- |
| | yl)phenyl]urea |
| 934 | 6-(1H-indol-5-yl)-4-morpholin-4-yl-1-(tetrahydro-2H-pyran-2-yl)-1H- |
| | pyrazolo[3,4-d]pyrimidine |
| 935 | 6-(1H-indol-5-yl)-4-morpholin-4-yl-1H-pyrazolo[3,4-d]pyrimidine |
| 936 | 5-[1-(1-benzylpiperidin-4-yl)-4-morpholin-4-yl-1H-pyrazolo[3,4- |
| | d]pyrimidin-6-yl]pyridin-3-ol |
| 937 | 1-(1-benzylpiperidin-4-yl)-6-[5-(methoxymethoxy)pyridin-3-yl]-4- |
| | morpholin-4-yl-1H-pyrazolo[3,4-d]pyrimidine |
| 938 | N-(4-{4-(2-hydroxymorpholin-4-yl)-1-[1-(pyridin-3- |
| | ylmethyl)piperidin-4-yl]-1H-pyrazolo[3,4-d]pyrimidin-6- |
| | yl}phenyl)acetamide |
| 939 | 1-(4-{4-(2-hydroxymorpholin-4-yl)-1-[1-(pyridin-3- |
| | ylmethyl)piperidin-4-yl]-1H-pyrazolo[3,4-d]pyrimidin-6-yl}phenyl)- |
| | 3-methylurea |
| 940 | 4-[4-morpholin-4-yl-1-(tetrahydro-2H-pyran-2-yl)-1H-pyrazolo[3,4- |
| | d]pyrimidin-6-yl]aniline |
| 941 | 1-methyl-3-{4-[4-morpholin-4-yl-1-(tetrahydro-2H-pyran-2-yl)-1H- |
| | pyrazolo[3,4-d]pyrimidin-6-yl]phenyl}urea |
| 942 | {4-[1-(1-benzylpiperidin-4-yl)-4-morpholin-4-yl-1H-pyrazolo[3,4- |
| | d]pyrimidin-6-yl]phenyl}acetic acid |
| 943 | 6-[1-(1-benzylpiperidin-4-yl)-4-morpholin-4-yl-1H-pyrazolo[3,4- |
| | d]pyrimidin-6-yl]quinoline |
| 944 | tert-butyl 4-[6-(4-aminophenyl)-4-morpholin-4-yl-1H-pyrazolo[3,4- |
| | d]pyrimidin-1-yl]piperidine-1-carboxylate |
| 945 | 1-methyl-3-{4-[4-morpholin-4-yl-1-(tetrahydro-2H-pyran-4-yl)-1H- |
| | pyrazolo[3,4-d]pyrimidin-6-yl]phenyl}urea |
| 946 | 1-[2-chloro-4-(4-morpholin-4-yl-1H-pyrazolo[3,4-d]pyrimidin-6- |
| | yl)phenyl]-3-methylurea |
| 947 | 1-(4-{4-[(2R,6S)-2,6-dimethylmorpholin-4-yl]-1-[1-(pyridin-3- |
| | ylmethyl)piperidin-4-yl]-1H-pyrazolo[3,4-d]pyrimidin-6-yl}phenyl)- |
| | 3-methylurea |
| 948 | 3-{4-[(3R)-3-methylmorpholin-4-yl]-1-phenyl-1H-pyrazolo[3,4- |
| | d]pyrimidin-6-yl}phenol |
| 949 | 3-[4-(2-methylmorpholin-4-yl)-1-phenyl-1H-pyrazolo[3,4- |
| | d]pyrimidin-6-yl]phenol |
| 950 | methyl 4-[6-(4-hydroxyphenyl)-4-morpholin-4-yl-1H-pyrazolo[3,4- |
| | d]pyrimidin-1-yl]piperidine-1-carboxylate |
| 951 | methyl 4-(4-morpholin-4-yl-6-{4-[(phenoxycarbonyl)amino]phenyl}- |
| | 1H-pyrazolo[3,4-d]pyrimidin-1-yl)piperidine-1-carboxylate |
| 952 | methyl 4-(6-{4-[(methylcarbamoyl)oxy]phenyl}-4-morpholin-4-yl- |
| | 1H-pyrazolo[3,4-d]pyrimidin-1-yl)piperidine-1-carboxylate |
| 953 | N-{4-[1-(1-isobutyrylpiperidin-4-yl)-4-morpholin-4-yl-1H- |
| | pyrazolo[3,4-d]pyrimidin-6-yl]phenyl}acrylamide |
| 954 | methyl 4-[6-(4-{[(4-fluorophenoxy)carbonyl]amino}phenyl)-4- |
| | morpholin-4-yl-1H-pyrazolo[3,4-d]pyrimidin-1-yl]piperidine-1- |
| | carboxylate |
| 955 | methyl 4-[6-(4-{[(Z)-(cyanoimino)(phenoxy)methyl]amino}phenyl)-4- |
| | morpholin-4-yl-1H-pyrazolo[3,4-d]pyrimidin-1-yl]piperidine-1- |
| | carboxylate |
| 956 | methyl 4-[6-(4-{[(4-chlorophenoxy)carbonyl]amino}phenyl)-4- |
| | morpholin-4-yl-1H-pyrazolo[3,4-d]pyrimidin-1-yl]piperidine-1- |
| | carboxylate |
| 957 | tert-butyl 4-(6-{4-[(methylsulfamoyl)amino]phenyl}-4-morpholin-4- |
| | yl-1H-pyrazolo[3,4-d]pyrimidin-1-yl)piperidine-1-carboxylate |
| 958 | tert-butyl 4-[6-(4-{[(6-fluoropyridin-3-yl)carbamoyl]amino}phenyl)- |
| | 4-morpholin-4-yl-1H-pyrazolo[3,4-d]pyrimidin-1-yl]piperidine-1- |
| | carboxylate |
| 959 | tert-butyl 4-{6-[4-({[4- |
| | (hydroxymethyl)phenyl]carbamoyl}amino)phenyl]-4-morpholin-4-yl- |
| | 1H-pyrazolo[3,4-d]pyrimidin-1-yl}piperidine-1-carboxylate |
| 960 | tert-butyl 4-{6-[4-({[4-(2- |
| | hydroxyethyl)phenyl]carbamoyl}amino)phenyl]-4-morpholin-4-yl-1H- |
| | pyrazolo[3,4-d]pyrimidin-1-yl}piperidine-1-carboxylate |
| 961 | 1-(4-{3-[3-(dimethylamino)prop-1-yn-1-yl]-1-ethyl-4-morpholin-4-yl- |
| | 1Hpyrazolo[3,4-d]pyrimidin-6-yl}phenyl)-3-methylurea |
| 962 | 1-{4-[1-ethyl-3-(3-hydroxyprop-1-yn-1-yl)-4-morpholin-4-yl-1H- |
| | pyrazolo[3,4-d]pyrimidin-6-yl]phenyl}-3-pyridin-3-ylurea |
| 963 | 4-{4-[6-(1H-indol-5-yl)-4-morpholin-4-yl-1H-pyrazolo[3,4- |
| | d]pyrimidin-1-yl]piperidin-1-yl}-N,N-dimethyl-4-oxobut-2-en-1- |
| | amine |
| 964 | N2,N2-dimethyl-N-[4-(4-morpholin-4-yl-1H-pyrazolo[3,4- |
| | d]pyrimidin-6-yl)phenyl]glycinamide |
| 965 | methyl (4-{1-[1-(4-fluorobenzyl)piperidin-4-yl]-4-morpholin-4-yl- |
| | 1Hpyrazolo[3,4-d]pyrimidin-6-yl}phenyl)carbamate |
| 966 | N-(4-{4-(2-hydroxymorpholin-4-yl)-1-[1-(pyridin-3- |
| | ylmethyl)piperidin-4-yl]-1Hpyrazolo[3,4-d]pyrimidin-6- |
| | yl}phenyl)acetamide |
| 967 | 1-(4-{4-(2-hydroxymorpholin-4-yl)-1-[1-(pyridin-3- |
| | ylmethyl)piperidin-4-yl]-1Hpyrazolo[3,4-d]pyrimidin-6-yl}phenyl)-3- |
| | methylurea |
| 968 | 3-[4-(1,4-oxazepan-4-yl)-1-phenyl-1H-pyrazolo[3,4-d]pyrimidin-6- |
| | yl]phenol |
| 969 | 3-(1-phenyl-4-thiomorpholin-4-yl-1H-pyrazolo[3,4-d]pyrimidin-6- |
| | yl)phenol |
| 970 | 3-(3-fluoro-4-morpholin-4-yl-1-phenyl-1H-pyrazolo[3,4-d]pyrimidin- |
| | 6-yl)phenol |

In another aspect, the invention provides compounds of Formula IIIb:

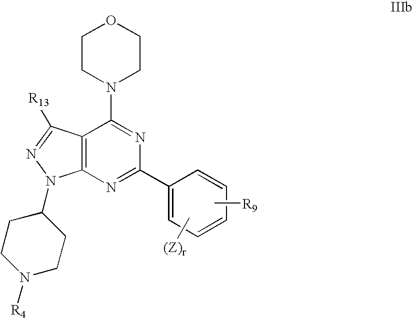

or a pharmaceutically acceptable salt or tautomer thereof, wherein R₄, R₉, Z, r, and R₁₃ are as defined above for the compounds of Formula IIIb.

In one embodiment, R₄ is C₁-C₈acyl, wherein the C₁-C₈acyl is optionally independently substituted with from 1 to 3 substituents as specified in Formula IIIb, heteroaryl(C₁-C₆alkyl) wherein the ring portion of the heteroaryl(C₁-C₆alkyl) group optionally independently substituted with from 1 to 3 substituents as specified in Formula IIIb, or (C₆-C₁₄aryl)alkyl, wherein the ring portion of the (C₆-C₁₄aryl)alkyl group is optionally independently substituted with from 1 to 3 substituents as specified in Formula IIIb.

In one embodiment, R₄ is C₁-C₈acyl, wherein the C₁-C₈acyl is optionally independently substituted with from 1 to 3 substituents as specified in Formula IIIb.

In one embodiment, R₄ is heteroaryl(C₁-C₆alkyl) wherein the ring portion of the heteroaryl(C₁-C₆alkyl) group is optionally independently substituted with from 1 to 3 substituents as specified in Formula IIIb or (C₆-C₁₄aryl)alkyl, wherein the ring portion of the (C₆-C₁₄aryl)alkyl group is optionally independently substituted with from 1 to 3 substituents as specified in Formula IIIb.

In one embodiment, R₉ is —NHC(O)NR₁₀R₁₁ or —NHC(O))R₁₂.

In one embodiment, R₁₀ is hydrogen and R₁₁ is selected from the group consisting of C₆-C₁₄aryl, C₁-C₉heteroaryl, C₃-C₈carbocycle, and C₁-C₆alkyl.

In one embodiment, R₁₁ is ethyl or 4-pyridyl.

In one embodiment, R₁₂ is C₁-C₆hydroxylalkyl.

In another aspect, the invention provides compounds of Formula IIIc:

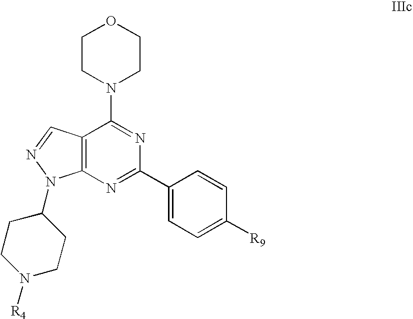

or a pharmaceutically acceptable salt or tautomer thereof, wherein R₄ and R₉ are as defined above for the compounds of Formula IIIc.

In one embodiment, R₄ is (C₁-C₆alkoxy)carbonyl optionally independently substituted with from 1 to 3 substituents as specified in Formula IIIc.

In one embodiment, R₄ is (C₁-C₆alkoxy)carbonyl.

In one embodiment, R₄ is R₄ is ethoxycarbonyl.

In one embodiment, R₄ is

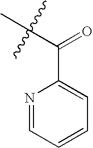

or a pharmaceutically acceptable salt thereof.

In one embodiment, R₄ is

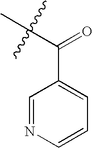

or a pharmaceutically acceptable salt thereof.

In one embodiment, R₄ is

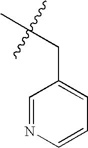

or a pharmaceutically acceptable salt thereof.

In one embodiment, R₄ is

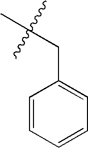

or a pharmaceutically acceptable salt thereof.

In one embodiment, R₁₉ is hydrogen.

In one embodiment, R₂₀ is C₁-C₆alkyl.

In one embodiment, R₂₀ is C₆-C₁₄aryl.

In one embodiment, R¹⁹ and R²⁰ when taken together with the nitrogen to which they are attached optionally form a 3- to 7- membered nitrogen-containing heterocycle wherein up to two of the carbon atoms of the heterocycle are optionally replaced with —N(H)—, —N(C₁-C₆alkyl)-, —N(C₆-C₁₄aryl)-, or —O—, and wherein the nitrogen-containing heterocycle is optionally substituted by a C₁-C₆alkyl; C₆-C₁₄aryl, (C₁-C₆alkoxy)C(O)NH—, or C₁-C₉heterocycle.

In one embodiment, R₉ is —NHC(O)NR₁₀R₁₁.

In one embodiment, R₉ is —NHC(O)OR₁₂.

In one embodiment, R₁₀ is hydrogen.

In one embodiment, R₁₁ is C₁-C₉heteroaryl or C₁-C₆alkyl.

In one embodiment, R₁₁ is C₁-C₆alkyl.

In one embodiment, R₁₁ is ethyl.

In one embodiment, R₁₁ is C₁-C₉heteroaryl.

In one embodiment, R₁₁ is pyridyl.

In one embodiment, R₁₁ is 4-pyridyl.

In one embodiment, R₉ is —NHC(O)OR₁₂.

In one embodiment, R₁₂ is C₁-C₆alkyl or C₁-C₆hydroxylalkyl.

In one embodiment, R₁₂ is C₁-C₆hydroxylalkyl.

In one embodiment, R₁₂ is hydroxylethyl.

In one embodiment, R₁₂ is propyl.

### Definitions

The following definitions are used in connection with the pyrazolopyrimidine analogs unless the context indicates otherwise. In general, the number of carbon atoms present in a given group is designated “Cₓ-C_{y}” n where x and y are the lower and upper limits, respectively. For example, a group designated as “C₁-C₆” contains from 1 to 6 carbon atoms. The carbon number as used in the definitions herein refers to carbon backbone and carbon branching, but does not include carbon atoms of the substituents, such as alkoxy substitutions and the like.

“Acyl” refers to a carbonyl group bonded to a moiety comprising a hydrogen atom or from 1 to 8 carbon atoms in a straight, branched, or cyclic configuration or a combination thereof, attached to the parent structure through the carbonyl functionality. The moiety may be saturated or unsaturated, aliphatic or aromatic, and carbocyclic or heterocyclic. Examples of C₁-C₈acyl include acetyl-, acryl-, benzoyl-, nicotinoyl, isonicotinyl N-oxide, propionyl-, isobutyryl-, oxalyl-, and the like. An acyl group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl.

“Alkoxy” refers to the group R—O— where R is an alkyl group, as defined below. Exemplary C₁-C₆alkoxy groups include but are not limited to methoxy, ethoxy, n-propoxy, 1-propoxy, n-butoxy and t-butoxy. An alkoxy group can be unsubstituted or substituted with one or more of the following groups: halogen, hydroxyl, C₁-C₆alkoxy, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, C₁-C₆alkoxy, —C(O)OH, —C(O)O(C1₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), C₁-C₆carboxyamidoalkyl-, or —NO₂.

“(Alkoxy)carbonyl” refers to the group alkyl-O—C(O)—. An (alkoxy)carbonyl group can be unsubstituted or substituted with one or more of the following groups: halogen, hydroxyl, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, C₁-C₆alkoxy, —C(O)OH, —C(O)O(C₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), C₁-C₆carboxyamidoalkyl-, or —NO₂. Exemplary (C₁-C₆alkoxy)carbonyl groups include but are not limited to CH₃—O—C(O)—, CH₃CH₂—O—C(O)—, CH₃CH₂CH₂—O—C(O)—, (CH₃)₂CH—O—C(O)—, and CH₃CH₂CH₂CH₂—O—C(O)—.

“Alkyl” refers to a hydrocarbon chain that may be a straight chain or branched chain, containing the indicated number of carbon atoms. For example, C₁-C₁₀ indicates that the group may have from 1 to 10 (inclusive) carbon atoms in it. In the absence of any numerical designation, “alkyl” is a chain (straight or branched) having 1 to 6 (inclusive) carbon atoms in it.

“C₁-C₃ alkyl” refers to a straight or branched chain saturated hydrocarbon containing 1-3 carbon atoms. Examples of a C₁-C₃ alkyl group include, but are not limited to, methyl, ethyl, propyl and isopropyl.

“C₁-C₆alkyl” refers to a straight or branched chain saturated hydrocarbon containing 1-5 carbon atoms. Examples of a C₁-C₆alkyl group include, but are not limited to, methyl, ethyl, propyl, isopropyl, n-pentyl, isopentyl, and neopentyl.

“C₁-C₆alkyl” refers to a straight or branched chain saturated hydrocarbon containing 1-6 carbon atoms. Examples of a C₁-C₆alkyl group include, but are not limited to, methyl, ethyl, propyl, butyl, pentyl, hexyl, isopropyl, isobutyl, sec-butyl, tert-butyl, isopentyl, neopentyl, and isohexyl.

“C₂-C₆ alkenyl” refers to a straight or branched chain unsaturated hydrocarbon containing 2-6 carbon atoms and at least one double bond. Examples of a C₂-C₆ alkenyl group include, but are not limited to, ethylene, propylene, 1-butylene, 2-butylene, isobutylene, sec-butylene, 1-pentene, 2-pentene, isopentene, 1-hexene, 2-hexene, 3-hexene, and isohexene.

“C₂-C₁₀alkenyl” refers to a straight or branched chain unsaturated hydrocarbon containing 2-10 carbon atoms and at least one double bond. Examples of a C₂-C₁₀ alkenyl group include, but are not limited to, ethylene, propylene, 1-butylene, 2-butylene, isobutylene, sec-butylene, 1-pentene, 2-pentene, isopentene, 1-hexene, 2-hexene, 3-hexene, isohexene, 1-heptene, 2-heptene, 3-heptene, 1-octene, 2-octene, 3-octene, 4-octene, 1-nonene, 2-nonene, 3-nonene, 4-nonene, 1-decene, 2-decene, 3-decene, 4-decene and 5-decene.

“Alkylene”, “alkenylene”, and “alkynylene” refers to the subsets of alkyl, alkenyl and alkynyl groups, as defined herein, including the same residues as alkyl, alkenyl, and alkynyl, but having two points of attachment within a chemical structure. Examples of C₁-C₆alkylene include methylene (—CH₂—), ethylene (—CH₂CH₂—), propylene (—CH₂CH₂CH₂—), and dimethylpropylene (—CH₂C(CH₃)₂CH₂—). Likewise, examples of C₂-C₆alkenylene include ethenylene (—CH═CH— and propenylene (—CH═CH—CH₂—). Examples of C₂-C₆alkynylene include ethynylene (—C≡C—) and propynylene (—C≡C—CH₂—).

“C_{2-C}₁₀alkynyl” refers to a straight or branched chain unsaturated hydrocarbon containing 2-10 carbon atoms and at least one triple bond. Examples of a C₂-C₁₀ alkynyl group include, but are not limited to, acetylene, propyne, 1-butyne, 2-butyne, isobutyne, sec-butyne, 1-pentyne, 2-pentyne, isopentyne, 1-hexyne, 2-hexyne, 3-hexyne, isohexyne, 1-heptyne, 2-heptyne, 3-heptyne, 1-octyne, 2-octyne, 3-octyne, 4-octyne, 1-nonyne, 2-nonyne, 3-nonyne, 4-nonyne, 1-decyne, 2-decyne, 3-decyne, 4-decyne and 5-decyne.

C_{3-C}₆ alkynyl” refers to a straight or branched chain unsaturated hydrocarbon containing 3-6 carbon atoms and at least one triple bond. Examples of a C₃-C₆ alkynyl group include, but are not limited to propyne, 1-butyne, 2-butyne, isobutyne, sec-butyne, 1-pentyne, 2-pentyne, isopentyne, 1-hexyne, 2-hexyne, 3-hexyne, and isohexyne.

“Alkylhalo” refers to a C₁-C₆alkyl group, as defined above, wherein one or more of the C₁-C₆alkyl group's hydrogen atoms has been replaced with —F, —Cl, —Br or —I. Each substitution can be independently selected from —F, —Cl, —Br, or —I. Representative examples of an C₁-C₆alkylhalo group include, but are not limited to —CH₂F, —CC₁₃, —CF₃, —CH₂Cl, —CH₂CH₂Br, —CH₂CH₂I, —CH₂CH₂CH₂F, —CH₂CH₂CH₂Cl, —CH₂CH₂CH₂CH₂Br, —CH₂CH₂CH₂CH₂ I, —CH₂CH₂CH₂CH₂CH₂Br, —CH₂CH₂CH₂CH₂CH₂I, —CH₂CH(Br)CH₃, —CH₂CH(Cl)CH₂CH₃, —CH(F)CH₂CH₃ and —C(CH₃)₂(CH₂Cl).

“Amino(alkyl)-” refers to an alkyl group, as defined above, wherein one or more of the alkyl group's hydrogen atoms has been replaced with —NH₂. Representative examples of an amino(C₁-C₆alkyl) group include, but are not limited to —CH₂NH₂, —CH₂CH₂NH₂, —CH₂CH₂CH₂ NH₂, —CH₂CH₂CH₂CH₂NH₂, —CH₂CH(NH₂)CH₃, —CH₂CH(NH₂)CH₂CH₃, —CH(NH₂)CH₂CH₃ and —C(CH₃)₂(CH₂NH₂), —CH₂CH₂CH₂CH₂CH₂NH₂, and —CH₂CH₂CH(NH₂)CH₂CH₃. An amino(alkyl) group can be unsubstituted or substituted with one or two of the following groups C₁-C₆alkoxy, C₆-C₁₄aryl, C₁-C₉heteroaryl, C₃-C₈cycloalkyl, and C₁-C₆alkyl.

“(Alkyl)amino-” refers to an —NH-alkyl group, where alkyl is as defined above. Representative examples of an (C₁-C₆alkyl)amino group include, but are not limited to —NHCH₃, —NHCH₂CH₃, —NHCH₂CH₂CH₃, —NHCH₂CH₂CH₂CH₃, —NHCH(CH₃)₂, —NHCH₂CH(CH₃)₂, —NHCH(CH₃)CH₂CH₃ and —NH—C(CH₃)₃. An (alkyl)amino group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C1₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), C₁-C₆carboxyamidoalkyl-, or —NO₂.

“Di(alkyl)amino-” refers to a nitrogen atom which has attached to it two alkyl groups, as defined above. Each alkyl group can be independently selected from the alkyl groups. Representative examples of an di(C₁-C₆alkyl)amino-group include, but are not limited to, —N(CH₃)₂, —N(CH₂CH₃)(CH₃), —N(CH₂CH₃)₂, —N(CH₂CH₂CH₃)₂, —N(CH₂CH₂CH₂CH₃)₂, —N(CH(CH₃)₂)₂, —N(CH(CH₃)₂)(CH₃), —N(CH₂CH(CH₃)₂)₂, —NH(CH(CH₃)CH₂CH₃)₂, —N(C(CH₃)₃)₂, —N(C(CH₃)₃)(CH₃), and —N(CH₃)(CH₂CH₃). The two alkyl groups on the nitrogen atom, when taken together with the nitrogen to which they are attached, can form a 3- to 7-membered nitrogen containing heterocycle wherein up to two of the carbon atoms of the heterocycle can be replaced with —N(R)—, —O—, or —S(O)ᵣ, —. R is hydrogen, C₁-C₆alkyl, C₃-C₈cycloalkyl, C₆-C₁₄aryl, C₁-C₉heteroaryl, amino(C₁-C₆alkyl), or arylamino. Variable r is 0, 1, or 2.

“Alkylcarboxy” refers to an alkyl group as defined above, attached to the parent structure through the oxygen atom of a carboxyl (C(O)—O—) functionality. Examples of C₁-C₆alkylcarboxy include acetoxy, ethylcarboxy, propylcarboxy, and isopentylcarboxy.

“(Alkyl)carboxyamido-” refers to a —NHC(O)— group in which the carbonyl carbon atom of said group is attached to an alkyl group, as defined above. Representative examples of a (C₁-C₆alkyl)carboxyamido group include, but are not limited to, —NHC(O)CH₃, —NHC(O)CH₂CH₃, —NHC(O)CH₂CH₂CH₃, —NHC(O)CH₂CH₂CH₂CH₃, —NHC(O)CH₂CH₂CH₂CH₂CH₃, —NHC(O)CH(CH₃)₂, —NHC(O)CH₂CH(CH₃)₂, —NHC(O)CH(CH₃)CH₂CH₃, —NHC(O)—C(CH₃)₃ and —NHC(O)CH₂C(CH₃)₃.

“(Aryl)amino” refers to a radical of formula aryl-NH—, wherein “aryl” is as defined below. Examples of (C₆-C₁₄aryl)amino radicals include, but are not limited to, phenylamino (anilido), 1-naphthlamino, 2-naphthlamino and the like. An (aryl)amino group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C1₁-C₆alkyl), —C(O)(C₁-C₆alkyl), C₆-C₁₄aryl, C₁-C₉heteroaryl, or C₃-C₈cycloalkyl.

“Aryl” refers to an aromatic hydrocarbon group. If not otherwise specified, in this specification the term aryl refers to a C₆-C₁₄aryl group. Examples of an C₆-C₁₄aryl group include, but are not limited to, phenyl, 1-naphthyl, 2-naphthyl, 3-biphen-1-yl, anthryl, tetrahydronaphthyl, fluorenyl, indanyl, biphenylenyl, and acenaphthenyl, groups. An aryl group can be unsubstituted or substituted with one or more of the following groups: C₁-C₆alkyl, C₃-C₈cycloalkyl, C₁-C₆perfluoroalkyl-, halo, haloalkyl-, hydroxyl, C₁-C₆hydroxylalkyl-, —NH₂, aminoalkyl-, dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

“(Aryl)alkyl” refers to an alkyl group, as defined above, wherein one or more of the alkyl group's hydrogen atoms has been replaced with a C₆-C₁₄aryl group as defined above. (C₆-C₁₄Aryl)alkyl moieties include benzyl, 1-phenylethyl, 2-phenylethyl, 3-phenylpropyl, 2-phenylpropyl, 1-naphthylmethyl, 2-naphthylmethyl and the like. An (aryl)alkyl group can be unsubstituted or substituted with one or more of the following groups: halogen, —NH₂, hydroxyl, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), C₁-C₆carboxyamidoalkyl-, or —NO₂.

“Heteroaryl” refers to mono, bicyclic, and tricyclic aromatic groups of 4 to 10 atoms containing at least one heteroatom and at least one aromatic ring. Heteroatom as used in the term heteroaryl refers to oxygen, sulfur and nitrogen. Examples of monocyclic C₁-C₉heteroaryls include, but are not limited to, pyrrolyl, oxazinyl, thiazinyl, pyridinyl, diazinyl, triazinyl, tetrazinyl, imidazolyl, tetrazolyl, isoxazolyl, furanyl, furazanyl, oxazolyl, thiazolyl, thiophenyl, pyrazolyl, triazolyl, and pyrimidinyl. Examples of bicyclic C₁-C₉heteroaryls include but are not limited to, benzimidazolyl, indolyl, indolinyl, isoquinolinyl, quinolinyl, quinazolinyl, benzothiophenyl, benzodioxolyl, benzo[1,2,5]oxadiazolyl, purinyl, benzisoxazolyl, benzoxazolyl, benzthiazolyl, benzodiazolyl, benzotriazolyl, isoindolyl and indazolyl. Examples of tricyclic C₁-C₁₃heteroaryls include but are not limited to, dibenzofuran, dibenzothiophenyl, phenanthridinyl, and benzoquinolinyl. Attachment of a heteroaryl substituent can occur via a carbon atom or via a nitrogen atom. Nitrogen-containing heteroaryl radicals also include the N-oxides thereof.

“Heteroaryl(alkyl)” refers to an alkyl group, as defined above, wherein one or more of the alkyl group's hydrogen atoms has been replaced with a heteroaryl group as defined above. Heteroaryl(C₁-C₆alkyl) moieties include 2-pyridylmethyl, 2-thiophenylethyl, 3-pyridylpropyl, 2-quinolinylmethyl, 2-indolylmethyl, and the like. A heteroaryl(alkyl) group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), monocyclic C₁-C₆heterocycle, C₆-C₁₄aryl, C₁-C₉heteroaryl, or C₃-C₈cycloalkyl.

“Arylamido” refers to an C₆-C₁₄aryl group, as defined above, wherein one of the C₆-C₁₄aryl group's hydrogen atoms has been replaced with one or more —C(O)NH₂ groups. Representative examples of a C₆-C₁₄arylamido group include 2-C(O)NH₂-phenyl, 3-C(O)NH₂-phenyl, 4-C(O)NH₂-phenyl, 2-C(O)NH₂-pyridyl, 3-C(O)NH₂-pyridyl and 4-C(O)NH₂-pyridyl.

“N-amidoalkyl” refers to a —NHC(O)— group in which the carbonyl carbon atom of said group is attached to a C₁-C₆alkyl group, as defined above. Representative examples of a N-amidoalkyl group include, but are not limited to, —NHC(O)CH₃, —NHC(O)CH₂CH₃, —NHC(O)CH₂CH₂CH₃, —NHC(O)CH₂CH₂CH₂CH₃, —NHC(O)CH₂CH₂CH₂CH₂CH₃, —NHC(O)CH(CH₃)₂, —NHC(O)CH₂CH(CH₃)₂, —NHC(O)CH(CH₃)CH₂CH₃, —NHC(O)—C(CH₃)₃ and —NHC(O)CH₂C(CH₃)₃.

“Carboxyamidoalkyl-” refers to a primary carboxyamide (—CONH₂), a secondary carboxyamide (CONHR′) or a tertiary carboxyamide (CONR′R″), where R′ and R″ are the same or different substituent groups selected from C₁-C₆alkyl, C₂-C₆alkenyl, C₂-C₆alkynyl, C₆-C₁₄aryl, C₁-C₉heteroaryl, or C₃-C₈cycloalkyl, attached to the parent compound by an alkyl group as defined above. Exemplary C₁-C₆carboxyamidoalkyl- groups include but are not limited to NH₂C(O)—CH₂—, CH₃NHC(O)—CH₂CH₂—, (CH₃)₂NC(O)—CH₂CH₂CH₂—, CH₂═CHCH₂NHC(O)—CH₂CH₂CH₂CH₂—, HCCCH₂NHC(O)—CH₂CH₂CH₂CH₂CH₂—, C₆H₅NHC(O)—CH₂CH₂CH₂CH₂CH₂CH₂—, 3-pyridylNHC(O)—CH₂CH(CH₃)CH₂CH₂—, and cyclopropyl-CH₂NHC(O)—CH₂CH₂C(CH₃)₂CH₂—.

A “C₃-C₈Carbocycle” is a non-aromatic, saturated hydrocarbon ring containing 3-8 carbon atoms. Representative examples of a C₃-C₈carbocycle include, but are not limited to, cyclopropyl, cyclobutyl, cyclopentyl, cyclohexyl, cycloheptyl and cyclooctyl. A C₃-C₈carbocycle can be unsubstituted or independently substituted with one or more of the following groups: —C₁-C₆alkyl, halo, -alkylhalo, hydroxyl, —O—C₁-C₆alkyl, —NH₂, -aminoalkyl, -aminodialkyl, —COOH, —C(O)O—(C₁-C₆alkyl), —OC(O)—(C₁-C₆alkyl), —N-amidoalkyl, —C(O)NH₂, -carboxyamidoalkyl or —NO₂.

“Halo” or halogen is —F, —Cl, —Br or —I.

The term “heteroatom” as used herein designates a sulfur, nitrogen, or oxygen atom.

“Heterocycle” or “heterocyclyl” refers to 3-10-membered mono and bicyclic groups containing at least one heteroatom selected from oxygen, sulfur and nitrogen. A heterocycle may be saturated or partially saturated. The sulfur atom may be in the (II) oxidation state, the sulfoxide oxidation state, or the sulfone oxidation state. The heterocyclic ring can be attached to the parent structure via a ring nitrogen or a ring carbon atom. Exemplary C₁-C₉heterocycle groups include but are not limited to aziridine, oxirane, thiirane, pyrroline, pyrrolidine, dihydrofuran, tetrahydrofuran, dihydrothiophene, tetrahydrothiophene, dithiolane, piperidine, tetrahydropyran, pyran, thiane, thiine, piperazine, morpholine, oxazine, thiazine, dithiane, dioxane, tetrahydroquinoline, and tetrahydroisoquinoline. Nitrogen-containing heterocycles also include the N-oxides thereof.

“Monocyclic heterocycle” refers to a monocyclic cycloalkyl, or cycloalkenyl in which 1-4 of the ring carbon atoms have been independently replaced with an N, O or S atom. The monocyclic heterocyclic ring can be attached to the parent structure via a ring nitrogen or a ring carbon atom. Representative examples of a monocyclic C₁-C₆heterocycle group include, but are not limited to, piperidinyl, 1,2,5,6-tetrahydropyridinyl, tetrahydrothiopyranyl, tetrahydrothiopyran-1-oxide, tetrahydrothiopyran-1,1-dioxide, piperazinyl, morpholinyl, oxazinyl, thiazinyl, pyrrolinyl, thinpyrrolidinyl, and homopiperidinyl. A monocyclic heterocycle group can be unsubstituted or substituted with one or more of the following groups: C₁-C₈acyl, C₁-C₆alkyl, heterocyclyl(C₁-C₆alkyl), (C₆-C₁₄aryl)alkyl, halo, C₁-C₆haloalkyl-, hydroxyl, C₁-C₆hydroxylalkyl-, —NH₂, aminoalkyl-, -dialkylamino-, —COOH, —C(O)O—(C₁-C₆alkyl), —OC(O)(C₁-C₆alkyl), (C₆-C₁₄aryl)alkyl-O—C(O)—, N-alkylamido-, —C(O)NH₂, (C₁-C₆alkyl)amido-, or —NO₂.

“Bicyclic heterocycle” refers to a bicyclic cycloalkyl or bicyclic cycloalkenyl in which 1-4 of the ring carbon atoms have been independently replaced with an N, O or S atom. The bicyclic heterocyclic ring can be attached via a nitrogen, sulfur, or carbon atom. Representative examples of a bicyclic C₁-C₉heterocycle group include, but are not limited to, indolinyl, tetrahydroisoquinolinyl, tetrahydroquinolinyl, and chromanyl. A bicyclic heterocycle group can be unsubstituted or substituted with one or more of the following groups: C₁-C₈acyl, C₁-C₆alkyl, heterocyclyl(C₁-C₆alkyl), (C₆-C₁₄aryl)alkyl, halo, C₁-C₆haloalkyl-, hydroxyl, C₁-C₆hydroxylalkyl-, —NH₂, aminoalkyl-, -dialkylamino-, —COOH, —C(O)O—(C₁-C₆alkyl), —OC(O)(C₁-C₆alkyl), (C₆-C₁₄aryl)alkyl-O—C(O)—, N-alkylamido-, —C(O)NH₂, (C₁-C₆alkyl)amido-, or —NO₂.

A “3- to 7-membered monocyclic heterocycle” refers to a monocyclic 3- to 7-membered aromatic or non-aromatic monocyclic cycloalkyl in which 1-4 of the ring carbon atoms have been independently replaced with an N, O or S atom. The 3- to 7-membered monocyclic heterocycles can be attached via a nitrogen, sulfur, or carbon atom. Representative examples of a 3- to 7-membered monocyclic C₁-C₆heterocyclee group include, but are not limited to, piperidinyl, piperazinyl, morpholinyl, pyrrolyl, oxazinyl, thiazinyl, diazinyl, triazinyl, tetrazinyl, imidazolyl, tetrazolyl, pyrrolidinyl, isoxazolyl, furanyl, furazanyl, pyridinyl, oxazolyl, thiazolyl, thiophenyl, pyrazolyl, triazolyl, and pyrimidinyl.

A “4- to 7-membered monocyclic heterocycle” refers to a monocyclic 4- to 7-membered aromatic or non-aromatic monocyclic cycloalkyl in which 1-4 of the ring carbon atoms have been independently replaced with an N, O or S atom. The 4- to 7-membered monocyclic heterocycles can be attached via a nitrogen, sulfur, or carbon atom. Representative examples of a 4- to 7-membered monocyclic C₁-C₆heterocycle group include, but are not limited to, piperidinyl, piperazinyl, morpholinyl, pyrrolyl, oxazinyl, thiazinyl, triazinyl, tetrazinyl, imidazolyl, tetrazolyl, pyrrolidinyl, isoxazolyl, furanyl, furazanyl, pyridinyl, oxazolyl, thiazolyl, thiophenyl, pyrazolyl, triazolyl, and pyrimidinyl.

A “nitrogen containing 3- to 7-membered monocyclic heterocycle” refers to a monocyclic 3- to 7-membered aromatic or non-aromatic monocyclic cycloalkyl group in which one of the cycloalkyl group's ring carbon atoms has been replaced with a nitrogen atom and 0-4 of the cycloalkyl group's remaining ring carbon atoms may be independently replaced with a N, O or S atom. Representative examples of nitrogen-containing-3- to 7-membered monocyclic C₁-C₆heterocycle include, but are not limited to, piperidinyl, piperazinyl, pyrrolyl, oxazinyl, thiazinyl, diazinyl, triazinyl, tetrazinyl, imidazolyl, tetrazolyl, pyrrolidinyl, isoxazolyl, pyridinyl, oxazolyl, thiazolyl, pyrazolyl, triazolyl, pyrimidinyl, and morpholinyl.

A “6- to 10-membered bicyclic heterocycle” refers to a bicyclic 6- to 10-membered aromatic or non-aromatic bicyclic cycloalkyl in which 1-4 of the ring carbon atoms have been independently replaced with an N, O or S atom. Representative examples of a 6- to 10-membered bicyclic heterocycle group include, but are not limited to, benzimidazolyl, indolyl, isoquinolinyl, indazolyl, quinolinyl, quinazolinyl, purinyl, benzisoxazolyl, benzoxazolyl, benzthiazolyl, benzodiazolyl, benzotriazolyl, isoindolyl and indazolyl.

A “7- to 10-membered bicyclic heterocycle” refers to a bicyclic 7- to 10-membered aromatic or non-aromatic bicyclic cycloalkyl in which 1-4 of the ring carbon atoms have been independently replaced with an N, O or S atom. Representative examples of a 7- to 10-membered bicyclic heterocycle group include, but are not limited to, benzimidazolyl, indolyl, isoquinolinyl, indazolyl, quinolinyl, quinazolinyl, purinyl, benzisoxazolyl, benzoxazolyl, benzthiazolyl, benzodiazolyl, benzotriazolyl, isoindolyl and indazolyl.

A “nitrogen-containing 7- to 10-membered bicyclic heterocycle” refers to a 7- to 10-membered bicyclic heterocycle, defined above, which contains at least one ring nitrogen atom. Representative nitrogen-containing 7- to 10-membered bicyclic heterocycles include -quinolinyl, -isoquinolinyl, -chromonyl, -indolyl, -isoindolyl, -indolizinyl, -indazolyl, -purinyl, -4H-quinolizinyl, -isoquinolyl, -quinolyl, -phthalazinyl, -naphthyridinyl -carbazolyl, -β-carbolinyl and the like.

“Heterocyclyl(alkyl)” refers to an alkyl group, as defined above, wherein one or more of the alkyl group's hydrogen atoms has been replaced with a heterocycle group as defined above. Heterocyclyl(C₁-C₆alkyl) moieties include 1-piperazinylethyl, 4-morpholinylpropyl, 6-piperazinylhexyl, and the like. A heterocyclyl(alkyl) group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), monocyclic C₁-C₆heterocycle, C₆-C₁₄aryl, C₁-C₉heteroaryl, or C₃-C₈cycloalkyl.

“Hydroxylalkyl-” refers to an alkyl group, as defined above, wherein one or more of the alkyl group's hydrogen atoms has been replaced with hydroxyl groups. Examples of C₁-C₆hydroxylalkyl- moieties include, for example, —CH₂OH, —CH₂CH₂OH, —CH₂CH₂CH₂OH, —CH₂CH(OH)CH₂O, —CH₂CH(OH)CH₃, —CH(CH₃)CH₂O and higher homologs.

“Perfluoroalkyl-” refers to a straight or branched chain hydrocarbon having two or more fluorine atoms. Examples of a C₁-C₆perfluoroalkyl-group include CF₃, CH₂CF₃, CF₂CF₃ and CH(CF₃)₂.

The term “optionally substituted” as used herein means that at least one hydrogen atom of the optionally substituted group has been substituted with halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, ——OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, or C₃-C₈carbocycle.

A “subject” is a mammal, e.g., a human, mouse, rat, guinea pig, dog, cat, horse, cow, pig, or non-human primate, such as a monkey, chimpanzee, baboon or rhesus.

As some of the compounds of the present invention possess an asymmetric carbon atom in the R₁ ring or the R₃ substituent, the present invention includes the racemate as well as the individual enantiomeric forms of the compounds of Formula I as described herein and in the claims. Mixtures of isomers of the compounds of the examples or chiral precursors thereof can be separated into individual isomers according to methods, which are known per se, e.g. fractional crystallization, adsorption chromatography or other suitable separation processes. Resulting racemates can be separated into antipodes in the usual manner after introduction of suitable salt-forming groupings, e.g. by forming a mixture of diastereosiomeric salts with optically active salt-forming agents, separating the mixture into diastereomeric salts and converting the separated salts into the free compounds. The enantiomeric forms may also be separated by fractionation through chiral high-pressure liquid chromatography columns.

The invention also includes pharmaceutical compositions comprising an effective amount of a Pyrazolopyrimidine Analog and a pharmaceutically acceptable carrier. The invention includes a Pyrazolopyrimidine Analog when provided as a pharmaceutically acceptable prodrug, hydrated salt, such as a pharmaceutically acceptable salt, or mixtures thereof.

Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts.

An “effective amount” when used in connection a Pyrazolopyrimidine Analog is an amount effective for treating or preventing a disease associated with mTOR.

The following abbreviations are used herein and have the indicated definitions: ACN is acetonitrile, AcOH is acetic acid, ATP is adenosine triphosphate, CHAPS is 3[(3-Cholamidopropyl)dimethylammonio]-propanesulfonic acid, DEAD is diethyl azodicarboxylate, DIAD is diisopropylazodicarboxylate, DMAP is dimethyl aminopyridine, DMF is N,N-dimethylformamide, DMSO is dimethylsulfoxide, DPBS is Dulbecco's Phosphate Buffered Saline Formulation, EDTA is ethylenediaminetetraacetic acid, ESI stands for Electrospray Ionization, EtOAc is ethyl acetate, EtOH is ethanol, HEPES is 4-(2-hydroxyethyl)-1-piperazineethanesulfonic acid, GMF is Glass, Hunig's Base is diisopropylethylamine, HPLC is high pressure liquid chromatography, LPS is lipopolysaccharide, MeCN is acetonitrile, MeOH is methanol, MS is mass spectrometry, NEt₃ is triethylamine, NMR is nuclear magnetic resonance, PBS is phosphate-buffered saline (pH 7.4), RPMI 1640 is a buffer (Sigma-Aldrich Corp., St. Louis, Mo., USA), SDS is dodecyl sulfate (sodium salt), SRB is Sulforhodamine B, TCA is tricholoroacetic acid, TFA is trifluoroacetic acid, THF is tetrahydrofuran, THP is the tetrahydro-2H-pyran-2-yl group, TLC is thin-layer chromatography, and TRIS is Tris(hydroxymethyl)aminomethane.

### Methods for Using the Pyrazolopyrimidine Analogs

The Pyrazolopyrimidine Analogs of the present invention exhibit an mTOR inhibitory activity and therefore, can be utilized in order to inhibit abnormal cell growth in which mTOR plays a role. Thus, the Pyrazolopyrimidine Analogs are effective in the treatment of disorders with which abnormal cell growth actions of mTOR are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the Pyrazolopyrimidine Analogs of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovary cancer, prostate cancer, lung cancer, colon cancer, pancreas cancer, renal cancer, gastric cancer, brain tumor, etc.

### Therapeutic Administration

When administered to an animal, the Pyrazolopyrimidine Analogs or pharmaceutically acceptable salts of the Pyrazolopyrimidine Analogs can be administered neat or as a component of a composition that comprises a physiologically acceptable carrier or vehicle. A composition of the invention can be prepared using a method comprising admixing the Pyrazolopyrimidine Analog or a pharmaceutically acceptable salt of the Pyrazolopyrimidine Analog and a physiologically acceptable carrier, excipient, or diluent. Admixing can be accomplished using methods well known for admixing a Pyrazolopyrimidine Analog or a pharmaceutically acceptable salt of the Pyrazolopyrimidine Analog and a physiologically acceptable carrier, excipient, or diluent.

The present compositions, comprising Pyrazolopyrimidine Analogs or pharmaceutically acceptable salts of the Pyrazolopyrimidine Analogs of the invention can be administered orally. The Pyrazolopyrimidine Analogs or pharmaceutically acceptable salts of Pyrazolopyrimidine Analogs of the invention can also be administered by any other convenient route, for example, by infusion or bolus injection, by absorption through epithelial or mucocutaneous linings (e.g., oral, rectal, vaginal, and intestinal mucosa, etc.) and can be administered together with another therapeutic agent. Administration can be systemic or local. Various known delivery systems, including encapsulation in liposomes, microparticles, microcapsules, and capsules, can be used.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result of release of the Pyrazolopyrimidine Analog or a pharmaceutically acceptable salt of the Pyrazolopyrimidine Analog into the bloodstream. The mode of administration is left to the discretion of the practitioner.

In one embodiment, the Pyrazolopyrimidine Analog or a pharmaceutically acceptable salt of the Pyrazolopyrimidine Analog is administered orally.

In another embodiment, the Pyrazolopyrimidine Analog or a pharmaceutically acceptable salt of the Pyrazolopyrimidine Analog is administered intravenously.

In another embodiment, it may be desirable to administer the Pyrazolopyrimidine Analog or a pharmaceutically acceptable salt of the Pyrazolopyrimidine Analog locally. This can be achieved, for example, by local infusion during surgery, topical application, e.g., in conjunction with a wound dressing after surgery, by injection, by means of a catheter, by means of a suppository or edema, or by means of an implant, said implant being of a porous, non-porous, or gelatinous material, including membranes, such as sialastic membranes, or fibers.

In certain embodiments, it can be desirable to introduce the Pyrazolopyrimidine Analog or a pharmaceutically acceptable salt of the Pyrazolopyrimidine Analog into the central nervous system, circulatory system or gastrointestinal tract by any suitable route, including intraventricular, intrathecal injection, paraspinal injection, epidural injection, enema, and by injection adjacent to the peripheral nerve. An intraventricular catheter, for example, can facilitate intraventricular injection attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the Pyrazolopyrimidine Analog or a pharmaceutically acceptable salt of the Pyrazolopyrimidine Analog can be formulated as a suppository, with traditional binders and excipients such as triglycerides.

In another embodiment, the Pyrazolopyrimidine Analog or a pharmaceutically acceptable salt of the Pyrazolopyrimidine Analog can be delivered in a vesicle, in particular a liposome (see Langer, Science 249:1527-1533 (1990) and Treat et al., Liposomes in the Therapy of Infectious Disease and Cancer pp. 317-327 and pp. 353-365 (1989)).

In yet another embodiment, the Pyrazolopyrimidine Analog or a pharmaceutically acceptable salt of the Pyrazolopyrimidine Analog can be delivered in a controlled-release system or sustained-release system (see, e.g., Goodson, in Medical Applications of Controlled Release, vol. 2, pp. 115-138 (1984)). Other controlled or sustained-release systems discussed in the review by Langer, Science 249:1527-1533 (1990) can be used. In one embodiment, a pump can be used (Langer, Science 249:1527-1533 (1990); Sefton, CRC Crit. Ref. Biomed. Eng. 14:201 (1987); Buchwald et al., Surgery 88:507 (1980); and Saudek et a t., N. Engl. J. Med. 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

In yet another embodiment, a controlled- or sustained-release system can be placed in proximity of a target of the Pyrazolopyrimidine Analog or a pharmaceutically acceptable salt of the Pyrazolopyrimidine Analog, e.g., the reproductive organs, thus requiring only a fraction of the systemic dose.

The present compositions can optionally comprise a suitable amount of a physiologically acceptable excipient.

Such physiologically acceptable excipients can be liquids, such as water and oils, including those of petroleum, animal, vegetable, or synthetic origin, such as peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one embodiment, the physiologically acceptable excipients are sterile when administered to an animal. The physiologically acceptable excipient should be stable under the conditions of manufacture and storage and should be preserved against the contaminating action of microorganisms. Water is a particularly useful excipient when the Pyrazolopyrimidine Analog or a pharmaceutically acceptable salt of the Pyrazolopyrimidine Analog is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

Liquid carriers may be used in preparing solutions, suspensions, emulsions, syrups, and elixirs. The Pyrazolopyrimidine Analog or pharmaceutically acceptable salt of the Pyrazolopyrimidine Analog of this invention can be dissolved or suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particular containing additives as above, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the composition is in the form of a capsule. Other examples of suitable physiologically acceptable excipients are described in Remington's Pharmaceutical Sciences pp. 1447-1676 (Alfonso R. Gennaro, ed., 19th ed. 1995).

In one embodiment, the Pyrazolopyrimidine Analog or a pharmaceutically acceptable salt of the Pyrazolopyrimidine Analog is formulated in accordance with routine procedures as a composition adapted for oral administration to humans. Compositions for oral delivery can be in the form of tablets, lozenges, buccal forms, troches, aqueous or oily suspensions or solutions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. In powders, the carrier can be a finely divided solid, which is an admixture with the finely divided Pyrazolopyrimidine Analog or pharmaceutically acceptable salt of the Pyrazolopyrimidine Analog. In tablets, the Pyrazolopyrimidine Analog or pharmaceutically acceptable salt of the Pyrazolopyrimidine Analog is mixed with a carrier having the necessary compression properties in suitable proportions and compacted in the shape and size desired. The powders and tablets can contain up to about 99% of the Pyrazolopyrimidine Analog or pharmaceutically acceptable salt of the Pyrazolopyrimidine Analog.

Capsules may contain mixtures of the Pyrazolopyrimidine Analogs or pharmaceutically acceptable salts of the Pyrazolopyrimidine Analogs with inert fillers and/or diluents such as pharmaceutically acceptable starches (e.g., corn, potato, or tapioca starch), sugars, artificial sweetening agents, powdered celluloses (such as crystalline and microcrystalline celluloses), flours, gelatins, gums, etc.

Tablet formulations can be made by conventional compression, wet granulation, or dry granulation methods and utilize pharmaceutically acceptable diluents, binding agents, lubricants, disintegrants, surface modifying agents (including surfactants), suspending or stabilizing agents (including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrroldine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine.

Moreover, when in a tablet or pill form, the compositions can be coated to delay disintegration and absorption in the gastrointestinal tract, thereby providing a sustained action over an extended period of time. Selectively permeable membranes surrounding an osmotically active driving compound or a pharmaceutically acceptable salt of the compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

In another embodiment, the Pyrazolopyrimidine Analog or a pharmaceutically acceptable salt of the Pyrazolopyrimidine Analog can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where the Pyrazolopyrimidine Analog or a pharmaceutically acceptable salt of the Pyrazolopyrimidine Analog is to be administered by infusion, it can be dispensed, for example, with an infusion bottle containing sterile pharmaceutical grade water or saline. Where the Pyrazolopyrimidine Analog or a pharmaceutically acceptable salt of the Pyrazolopyrimidine Analog is administered by injection, an ampoule of sterile water for injection or saline can be provided so that the ingredients can be mixed prior to administration.

In another embodiment, the Pyrazolopyrimidine Analog or pharmaceutically acceptable salt of the Pyrazolopyrimidine Analog can be administered transdermally through the use of a transdermal patch. Transdermal administrations include administrations across the surface of the body and the inner linings of the bodily passages including epithelial and mucosal tissues. Such administrations can be carried out using the present Pyrazolopyrimidine Analogs or pharmaceutically acceptable salts of the Pyrazolopyrimidine Analogs, in lotions, creams, foams, patches, suspensions, solutions, and suppositories (e.g., rectal or vaginal).

Transdermal administration can be accomplished through the use of a transdermal patch containing the Pyrazolopyrimidine Analog or pharmaceutically acceptable salt of the Pyrazolopyrimidine Analog and a carrier that is inert to the Pyrazolopyrimidine Analog or pharmaceutically acceptable salt of the Pyrazolopyrimidine Analog, is non-toxic to the skin, and allows delivery of the agent for systemic absorption into the blood stream via the skin. The carrier may take any number of forms such as creams or ointments, pastes, gels, or occlusive devices. The creams or ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the Pyrazolopyrimidine Analog or pharmaceutically acceptable salt of the Pyrazolopyrimidine Analog into the blood stream, such as a semi-permeable membrane covering a reservoir containing the Pyrazolopyrimidine Analog or pharmaceutically acceptable salt of the Pyrazolopyrimidine Analog with or without a carrier, or a matrix containing the active ingredient.

The Pyrazolopyrimidine Analogs or pharmaceutically acceptable salts of the Pyrazolopyrimidine Analogs of the invention may be administered rectally or vaginally in the form of a conventional suppository. Suppository formulations may be made from traditional materials, including cocoa butter, with or without the addition of waxes to alter the suppository's melting point, and glycerin. Water-soluble suppository bases, such as polyethylene glycols of various molecular weights, may also be used.

The Pyrazolopyrimidine Analog or a pharmaceutically acceptable salt of the Pyrazolopyrimidine Analog can be administered by controlled-release or sustained-release means or by delivery devices that are known to those of ordinary skill in the art. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release. Advantages of controlled- or sustained-release compositions include extended activity of the drug, reduced dosage frequency, and increased compliance by the animal being treated. In addition, controlled- or sustained-release compositions can favorably affect the time of onset of action or other characteristics, such as blood levels of the Pyrazolopyrimidine Analog or a pharmaceutically acceptable salt of the Pyrazolopyrimidine Analog, and can thus reduce the occurrence of adverse side effects.

Controlled- or sustained-release compositions can initially release an amount of the Pyrazolopyrimidine Analog or a pharmaceutically acceptable salt of the Pyrazolopyrimidine Analog that promptly produces the desired therapeutic or prophylactic effect, and gradually and continually release other amounts of the Pyrazolopyrimidine Analog or a pharmaceutically acceptable salt of the Pyrazolopyrimidine Analog to maintain this level of therapeutic or prophylactic effect over an extended period of time. To maintain a constant level of the Pyrazolopyrimidine Analog or a pharmaceutically acceptable salt of the Pyrazolopyrimidine Analog in the body, the Pyrazolopyrimidine Analog or a pharmaceutically acceptable salt of the Pyrazolopyrimidine Analog can be released from the dosage form at a rate that will replace the amount of the Pyrazolopyrimidine Analog or a pharmaceutically acceptable salt of the Pyrazolopyrimidine Analog being metabolized and excreted from the body. Various conditions, including but not limited to, changes in pH, changes in temperature, concentration or availability of enzymes, concentration or availability of water, or other physiological conditions or Pyrazolopyrimidine Analogs can stimulate controlled- or sustained-release of an active ingredient.

In certain embodiments, the present invention is directed to prodrugs of the Pyrazolopyrimidine Analogs or pharmaceutically acceptable salts of Pyrazolopyrimidine Analogs of the present invention. Various forms of prodrugs are known in the art, for example as discussed in Bundgaard (ed.), Design of Prodrugs, Elsevier (1985); Widder et al. (ed.), Methods in Enzymology, vol. 4, Academic Press (1985); Kgrogsgaard-Larsen et al. (ed.); “Design and Application of Prodrugs”, Textbook of Drug Design and Development, Chapter 5, 113-191(1991); Bundgaard et al., Journal of Drug Delivery Reviews, 8:1-38 (1992); Bundgaard et al., J. Pharmaceutical Sciences, 77:285 et seq. (1988); and Higuchi and Stella (eds.), Prodrugs as Novel Drug Delivery Systems, American Chemical Society (1975).

The amount of the Pyrazolopyrimidine Analog or a pharmaceutically acceptable salt of the Pyrazolopyrimidine Analog that is effective for treating or preventing an mTOR-related disorder. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The effective dosage amounts described herein refer to total amounts administered; that is, if more than one Pyrazolopyrimidine Analog or a pharmaceutically acceptable salt of the Pyrazolopyrimidine Analog is administered, the effective dosage amounts correspond to the total amount administered.

The amount of the Pyrazolopyrimidine Analog or a pharmaceutically acceptable salt of the Pyrazolopyrimidine Analog that is effective for treating or preventing an mTOR-related disorder will typically range from about 0.001 mg/kg to about 250 mg/kg of body weight per day, in one embodiment, from about 1 mg/kg to about 250 mg/kg body weight per day, in another embodiment, from about 1 mg/kg to about 50 mg/kg body weight per day, and in another embodiment, from about 1 mg/kg to about 20 mg/kg of body weight per day.

In one embodiment, the pharmaceutical composition is in unit dosage form, e.g., as a tablet, capsule, powder, solution, suspension, emulsion, granule, or suppository. In such form, the composition is sub-divided in unit dose containing appropriate quantities of the active ingredient; the unit dosage form can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain from about 1 mg/kg to about 250 mg/kg, and may be given in a single dose or in two or more divided doses.

The Pyrazolopyrimidine Analog or a pharmaceutically acceptable salt of the Pyrazolopyrimidine Analog can be assayed in vitro or in vivo for the desired therapeutic or prophylactic activity prior to use in humans. Animal model systems can be used to demonstrate safety and efficacy.

The present methods for treating or preventing an mTOR-related disorder, can further comprise administering another therapeutic agent to the animal being administered the Pyrazolopyrimidine Analog or a pharmaceutically acceptable salt of the Pyrazolopyrimidine Analog. In one embodiment, the other therapeutic agent is administered in an effective amount.

Effective amounts of the other therapeutic agents are well known to those skilled in the art. However, it is well within the skilled artisan's purview to determine the other therapeutic agent's optimal effective amount range. The Pyrazolopyrimidine Analog or a pharmaceutically acceptable salt of the Pyrazolopyrimidine Analog and the other therapeutic agent can act additively or, in one embodiment, synergistically. In one embodiment, of the invention, where another therapeutic agent is administered to an animal, the effective amount of the Pyrazolopyrimidine Analog or a pharmaceutically acceptable salt of the Pyrazolopyrimidine Analog is less than its effective amount would be where the other therapeutic agent is not administered. In this case, without being bound by theory, it is believed that the Pyrazolopyrimidine Analog or a pharmaceutically acceptable salt of the Pyrazolopyrimidine Analog and the other therapeutic agent act synergistically.

Suitable other therapeutic agents useful in the methods and compositions of the present invention include, but are not limited to temozolomide, a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, taxanes such as docetaxel and paclitaxel, leucovorin, levamisole, irinotecan, estramustine, etoposide, nitrogen mustards, BCNU, nitrosoureas such as carmustine and lomustine, vinca alkaloids such as vinblastine, vincristine and vinorelbine, platinum complexes such as cisplatin, carboplatin and oxaliplatin, imatinib mesylate, Avastin (Bevacizumab), hexamethylmelamine, topotecan, tyrosine kinase inhibitors, tyiphostins, herbimycin A, genistein, erbstatin, and lavendustin A.

Other therapeutic agents useful in the methods and compositions of the present invention include, but are not limited to hydroxyzine, glatiramer acetate, interferon beta-1a, interferon beta-1b, mitoxantrone, and natalizumab.

In one embodiment, the Pyrazolopyrimidine Analog or a pharmaceutically acceptable salt of the Pyrazolopyrimidine Analog is administered concurrently with another therapeutic agent.

In one embodiment, a composition comprising an effective amount of the Pyrazolopyrimidine Analog or a pharmaceutically acceptable salt of the Pyrazolopyrimidine Analog and an effective amount of another therapeutic agent within the same composition can be administered.

In another embodiment, a composition comprising an effective amount of the Pyrazolopyrimidine Analog or a pharmaceutically acceptable salt of the Pyrazolopyrimidine Analog and a separate composition comprising an effective amount of another therapeutic agent can be concurrently administered. In another embodiment, an effective amount of the Pyrazolopyrimidine Analog or a pharmaceutically acceptable salt of the Pyrazolopyrimidine Analog is administered prior to or subsequent to administration of an effective amount of another therapeutic agent. In this embodiment, the Pyrazolopyrimidine Analog or a pharmaceutically acceptable salt of the Pyrazolopyrimidine Analog is administered while the other therapeutic agent exerts its therapeutic effect, or the other therapeutic agent is administered while the Pyrazolopyrimidine Analog or a pharmaceutically acceptable salt of the Pyrazolopyrimidine Analog exerts its preventative or therapeutic effect for treating or preventing an mTOR-related disorder.

In another embodiment, the pharmaceutically acceptable carrier is suitable for oral administration and the composition comprises an oral dosage form.

In one embodiment, a method of inhibiting mTOR in a subject, comprising administering to a subject in need thereof a compound of Formula (I), Formula (Ia), Formula (II), Formula (III), Formula (IIIa), Formula (IIIb), and Formula (IIIc) in an amount effective to inhibit mTOR.

In one embodiment, a method of inhibiting PI3K in a subject, comprising administering to a subject in need thereof a compound of Formula (I), Formula (Ia), Formula (II), Formula (III), Formula (IIIa), Formula (IIIb), and Formula (IIIc) in an amount effective to inhibit PI3K.

The Pyrazolopyrimidine Analogs and pharmaceutically acceptable salts of Pyrazolopyrimidine Analogs can be prepared using a variety of methods starting from commercially available compounds, known compounds, or compounds prepared by known methods. General synthetic routes to many of the compounds of the invention are included in the following schemes. It is understood by those skilled in the art that protection and deprotection steps not shown in the Schemes may be required for these syntheses, and that the order of steps may be changed to accommodate functionality in the target molecule.

Methods useful for making the Pyrazolopyrimidine Analogs are set forth in the Examples below and generalized in Schemes 1-62. Reasonable variations of the described procedures, which would be evident to one skilled in the art, are intended to be within the scope of the present invention:

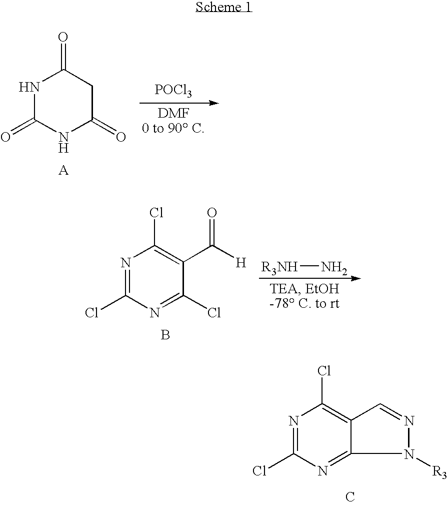

wherein R₃ is as defined above for the Pyrazolopyrimidine Analogs of Formula (I), Formula (Ia), Formula (II), Formula (III), Formula (IIIa), Formula (IIIb), and Formula (IIIc).

As shown in Scheme 1, a compound of formula C may be prepared by reacting a compound of formula A with a chlorinating agent, for example, POCl₃, in a polar aprotic solvent, such as DMF, and then subjecting the chloroaldehyde B to a hydrazine derivative. The hydrazine derivative may be purchased or prepared via standard organic chemistry protocols.

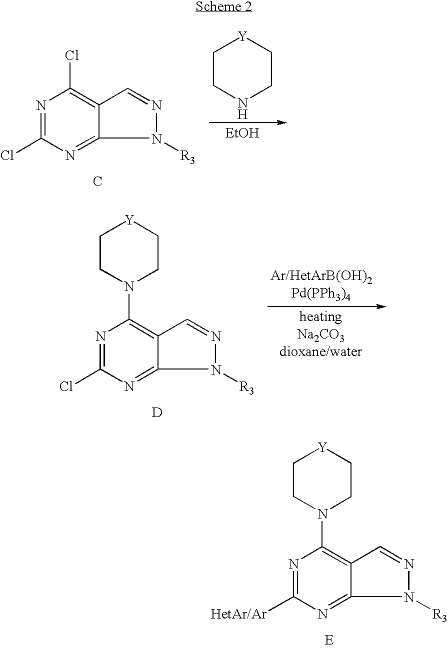

wherein Y is selected from the group consisting of —CH—, —S—, —O—, or —N—P, wherein P is a suitable protecting group.

As set forth in Scheme 2, a compound of formula E, for example, a 4-amino-6-aryl/heteroaryl pyrazolopyrimidine, may be prepared by reacting a compound of formula D with an amine in a protic solvent, for example, ethanol. A compound of formula D can then be reacted with a boronic acid such as an aryl or heteroaryl boronic acid under Suzuki reaction conditions (Miyaura, N and Suzuki, A., Chem. Rev., 95, 2457 (1995)) to give a compound of formula E.

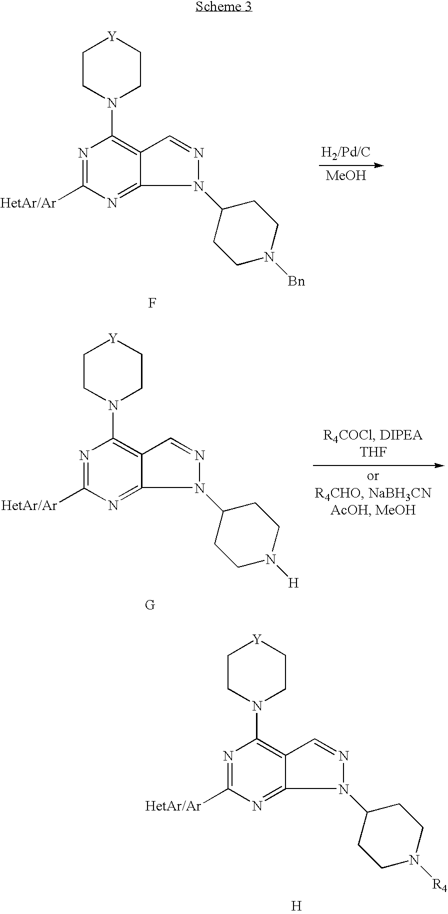

wherein A, B, and R₄ are as defined above and Y is as defined in Scheme 2.

As set forth in Scheme 3, a compound of formula F can be hydrogenated under catalytic conditions, for example, H₂/Pd/C in methanol, to remove the benzyl group from the piperidine to give a compound of formula G wherein R₄ is H. The free nitrogen of the piperidine of a compound of formula G can be converted into an amide through reaction with an acyl chloride and DIPEA in a polar aprotic solvent, for example, THF, or be converted into an alkyl amine through reaction with an aldehyde followed by reduction, with for example, sodium cyanoborohydride, to give a compound of the formula H.

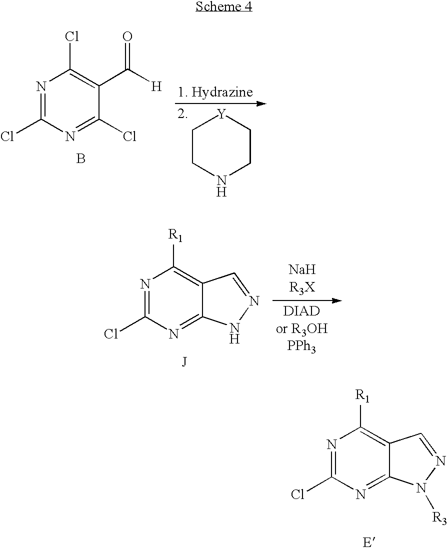

wherein Y is selected from the group consisting of —CH—, —S—, —O—, or —N—P, wherein P is a suitable protecting group, and R₁ and R₃ are as defined above.

As set forth in Scheme 4, reaction of the trichloroaldehyde B in a polar solvent, for example, ethanol, with hydrazine followed by the addition of morpholine will give a compound of the formula J. The compound of formula J can be N-alkylated under Mitsunobu reaction conditions with an alcohol or by treatment with sodium hydride and an alkyl halide under microwave irradiation at 175° C. for 10 minutes followed by purification by, for example, reverse phase HPLC to give a compound of formula E′. Alternatively, reacting a compound of formula J under Mitsunobu conditions can alkylate the pyrazole ring nitrogen atom.

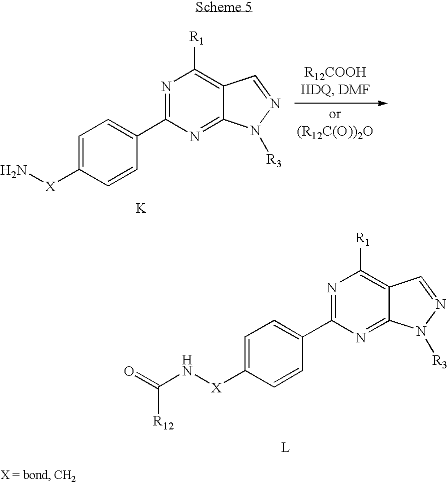

wherein R₁, R₃, and R₁₂ are as defined above.

As set forth in Scheme 5, treatment of the NH₂ group with a carboxylic acid and IIDQ in DMF at room temperature or with an anhydride (with pyridine and a catalytic amount of DMAP) at 50° C. will give a compound of formula L.

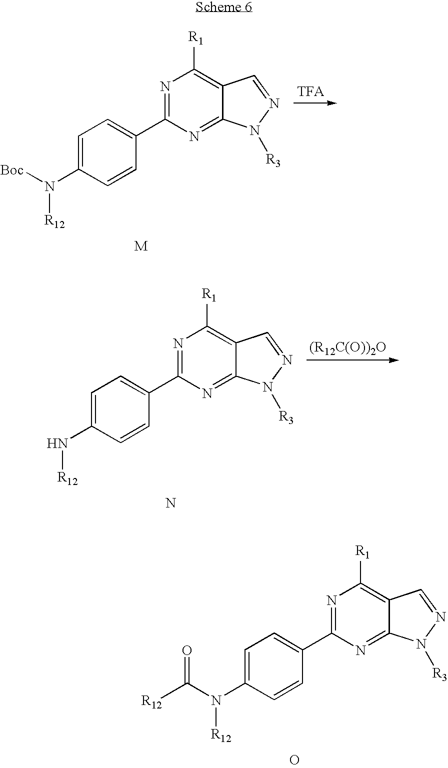

wherein R₁, R₃, and R₁₂ are as defined above.

As set forth in Scheme 6, the BOC protecting group of a compound of formula M can be removed by treatment with TFA to give a compound of formula N. Treatment with an anhydride under the appropriate conditions (for example pyridine and DMAP) will give a compound of formula O.

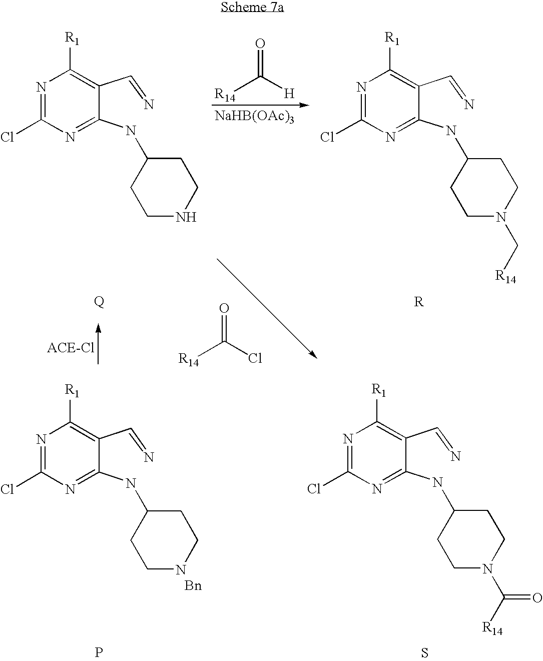

wherein R , is as described above, and R₁₄ is hydrogen, optionally substituted C₁-C₆alkyl, optionally substituted —C(O)alkyl, optionally substituted —C(O)alkoxy, optionally substituted —C(O)NR₅R₆, optionally substituted C₆-C₁₄aryl, or optionally substituted heterocycle.

As set forth in Scheme 7a, treatment of a compound of formula P with alpha-chloroethyl chloroformate, ACE Cl, will give a compound of formula Q which can be reacted with a acylchloride to give a compound of formula R. Alternatively, a compound of formula Q can be reacted with a carbaldehyde and NaHB(OAc)₃ to give a compound of the formula S.

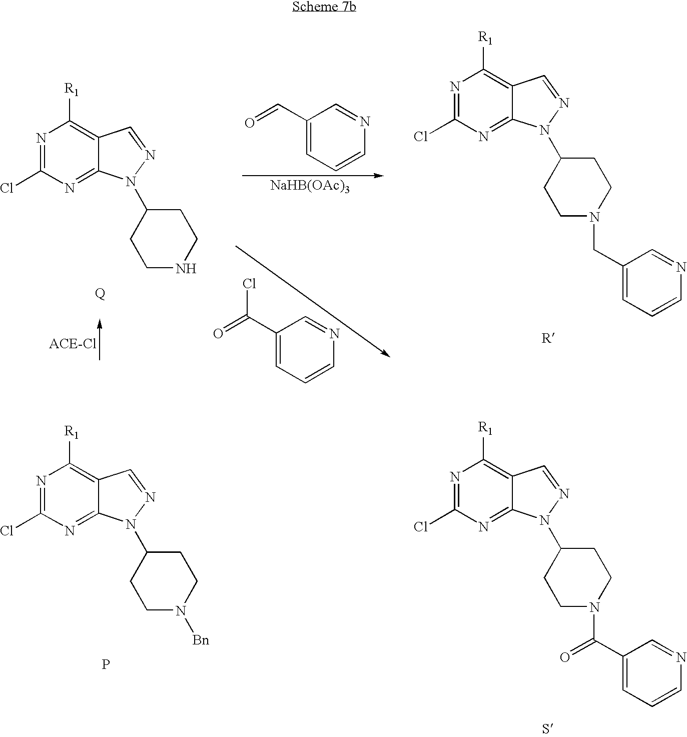

wherein R₁ is defined as above.

As set forth in Scheme 7b, treatment of a compound of formula P with alpha-chloroethyl chloroformate, ACE Cl, will give a compound of formula Q which can be reacted with a nicotinoyl chloride to give a compound of formula R′Alternatively, a compound of formula Q can be reacted with a pyridine-3-carbaldehyde and NaHB(OAc)₃ to give a compound of the formula S′.

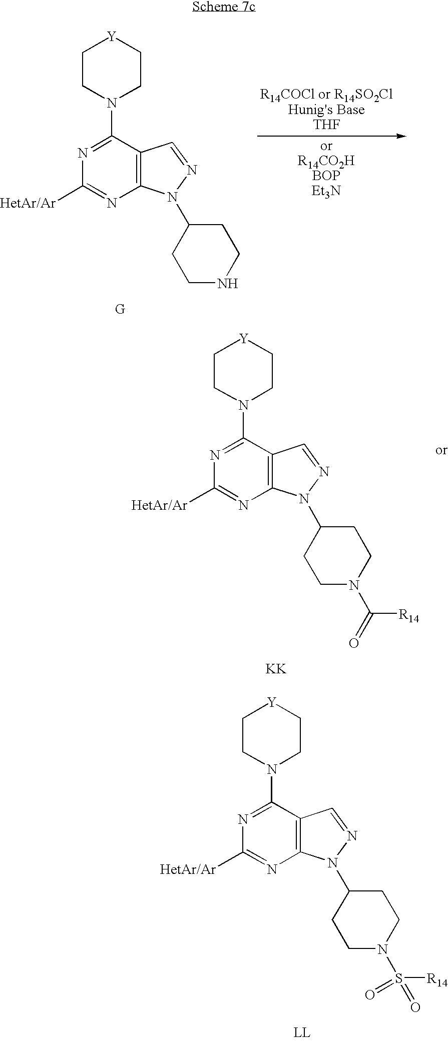

wherein R₁₄ is as defined in Scheme 7a and Y is as defined in Scheme 2.

As set forth in Scheme 7c, a compound of formula KK can be prepared by reacting a compound of formula G with an acylchloride in the presence of Hunig's base (diisopropylethylamine) in THF or with a carboxylic acid and BOP in the presence of triethylamine. Alternatively, a compound of formula LL can be formed by reacting a compound of formula G with a sulfonylchloride in the presence of Hunig's base in THF.

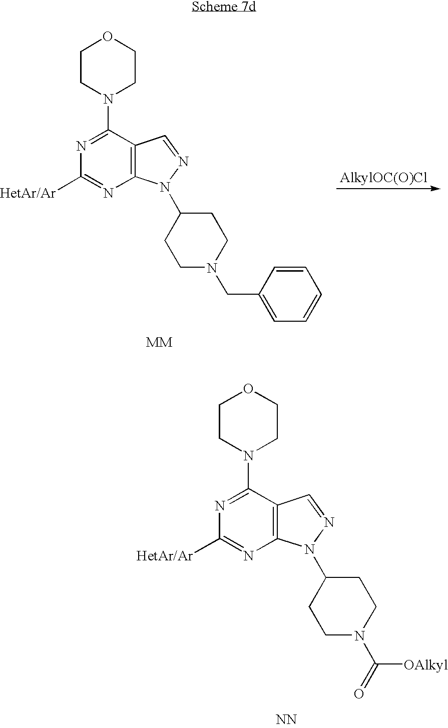

As set forth in Scheme 7d, a compound of formula NN can be formed by reacting a compound of formula MM with an alkyl chloroformate.

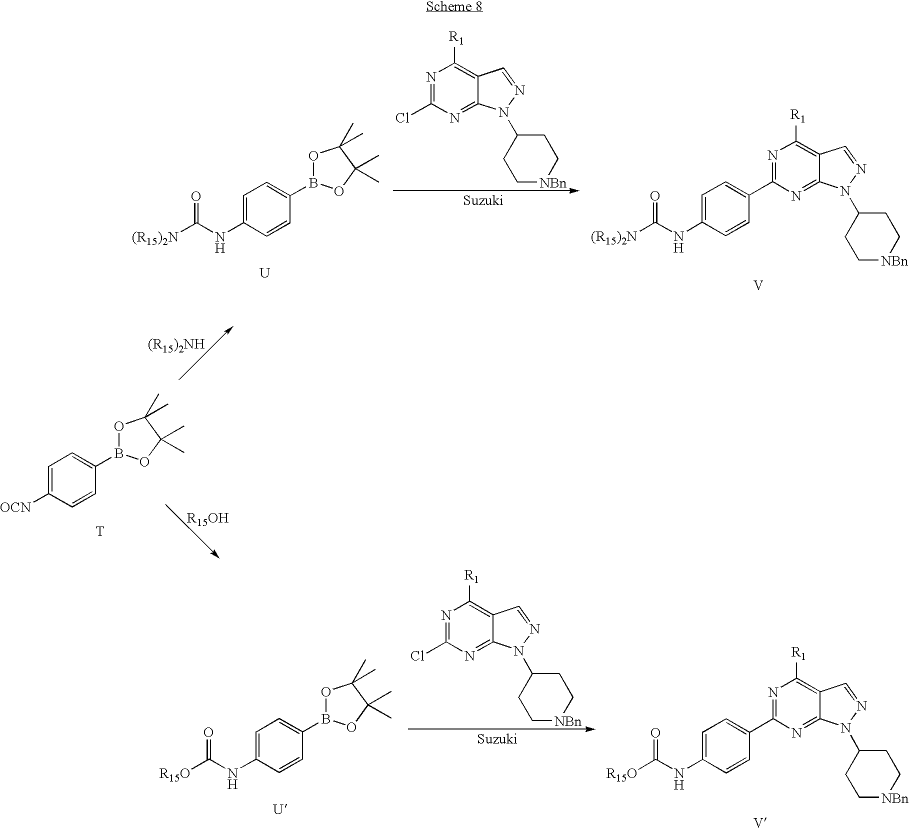

wherein R₁ is as defined above and each R₁₅ is independently hydrogen, optionally substituted C₁-C₆alkyl, optionally substituted alkenyl, optionally substituted alkynyl, optionally substituted C₃-C₈carbocycle, optionally substituted C₆-C₁₄aryl, or optionally substituted heteroaryl.

As set forth in Scheme 8, a compound of formula T can be treated with an alcohol to give a compound of the formula U′. Alternatively, a compound of formula T can be treated with an amine to give a urea of the formula U. A compound of the formula U can be reacted under Suzuki conditions with a chloropyrazoloyrimidine to give a compound of formula V. Alternatively, a compound of formula U′ can be reacted under Suzuki conditions with a chloropyrazoloyrimidine to give a compound of formula V′.

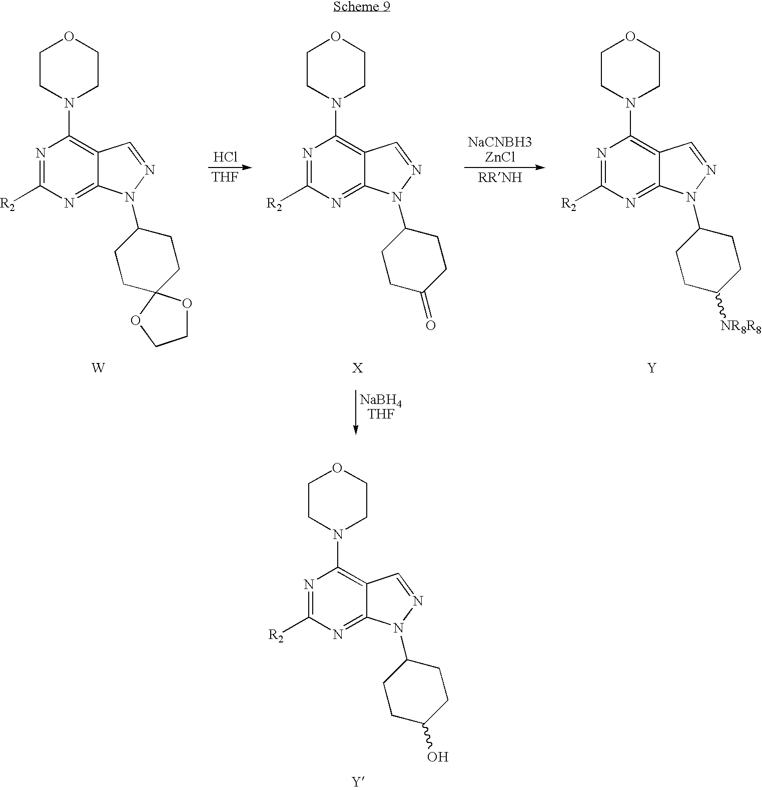

wherein R₂ is as defined above, and R₁₅ is as defined in Scheme 8.

As set forth in Scheme 9, a compound of formula W can be treated with an acid, for example, HCl, in THF to give a ketone compound of the formula X. The ketone compound of formula X can be treated with an amine and NaCNBH₃ and ZnCl₂ to give an amine compound of the formula Y. Alternatively, a ketone compound of formula X can be treated with a reducing agent, for example NaBH₄, providing the alcohol compound of formula Y′.

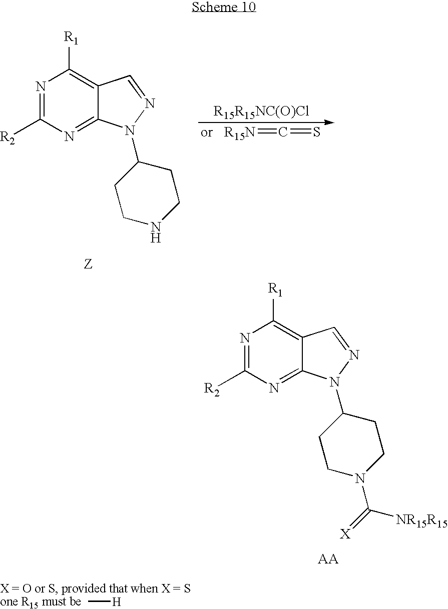

wherein R₁ and R₂ are as defined above, and R₁₅ is as defined in Scheme 8.

As set forth in Scheme 10, a compound of formula Z can be treated with a carbamyl chloride in dichloromethane and excess triethylamine at room temperature for 10 min. to give a urea compound of formula AA where X=0. Alternatively, a compound of formula Z can be treated with an isothiocyanate in dichloromethane and excess triethylamine at room temperature for 10 min. to give a thiourea compound of formula AA where X═S.

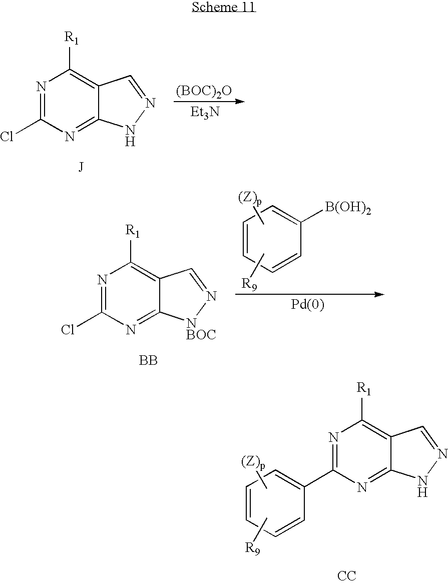

wherein R₁, R₉, Z and q are as defined above.

As set forth in Scheme 11, the pyrazole nitrogen of a compound of formula J can be BOC protected by treatment with (BOC)₂O and excess Et₃N to give a compound of formula BB. The resulting compound of formula BB can be coupled with an aryl boronic acid to give a compound of formula CC with concomitant loss of the BOC group.

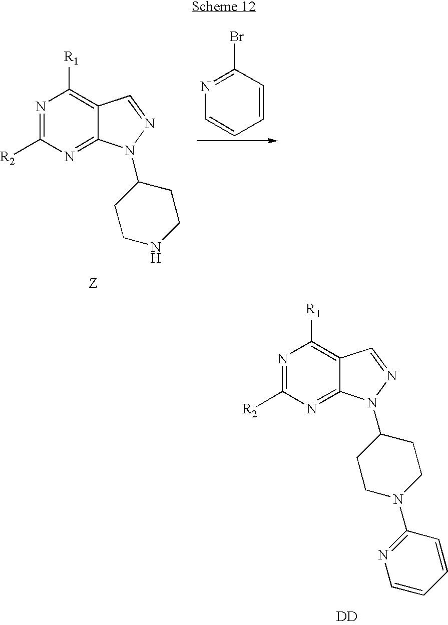

wherein R₁ and R₂ are as defined above.

As set forth in Scheme 12, a compound of formula Z can be heated with either 2- or 4-bromopyridine at 100° C. for three days to give a 2-piperidinyl pyridine compound of formula DD.

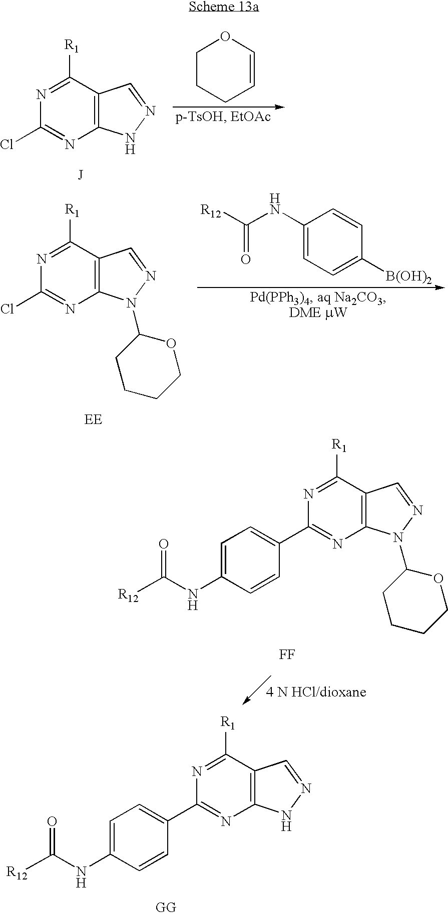

wherein R₁ and R₁₂ are as defined above.

As set forth in Scheme 13a, the pyrazole nitrogen of a compound of formula J can be protected with a tetrahydropyran ring by treatment with dihydopyran in the presence of para-toluenesulfonic acid at 60° C. for 18 hours to give a compound of formula EE. Suzuki coupling of a compound of formula EE with an arylboronic acid (as described in Scheme 2) will give a compound of formula FF. The pyrazole nitrogen of a compound of formula FF can be deprotected by treatment with HCl in dioxane to give a compound of formula GG. Although the structure shown is a phenyl ring, the aryl moiety of the arylboronic acid can also be a pyridyl moiety, a pyrimidine moiety, or a pyrazine, moiety. Furthermore, it is contemplated that heteroaryl moieties may also be substituted for the phenyl ring in the arylboronic acids of scheme 13a.

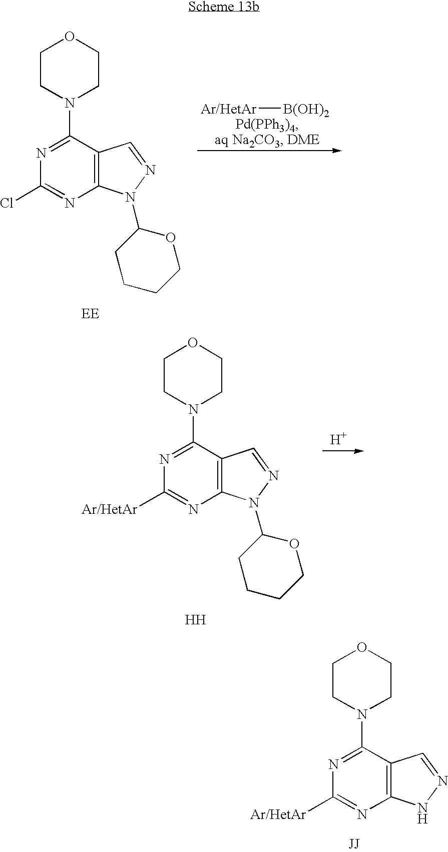

As set forth in Scheme 13b, a compound of formula EE can be reacted with a boronic acid such as an aryl or heteroaryl boronic acid under Suzuki reaction conditions (Miyaura, N and Suzuki, A., Chem. Rev., 95, 2457 (1995)) to give a compound of formula HH. The pyrazole nitrogen of a compound of formula HH can be deprotected by treatment with HCl in dioxane to give a compound of formula JJ.

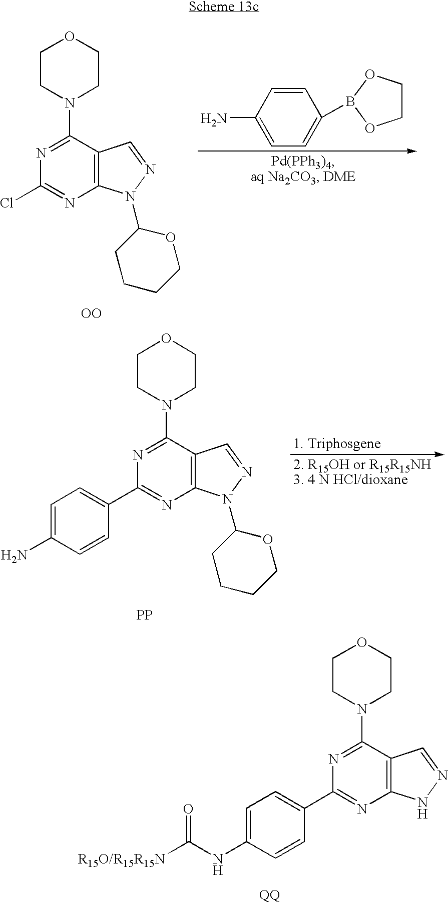

wherein R₁₅ is as defined in Scheme 8.

As set forth in Scheme 13c, a compound of formula OO can be reacted with a boronic acid such as an aryl or heteroaryl boronic acid under Suzuki reaction conditions (Miyaura, N and Suzuki, A., Chem. Rev., 95, 2457 (1995)) to give a compound of formula PP. The pyrazole nitrogen of a compound of formula PP can be deprotected by treatment with HCl in dioxane to give a compound of formula OO

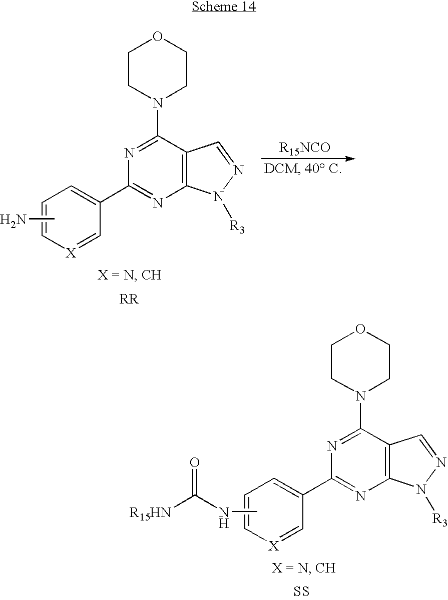

wherein R₃ is as defined above and R₁₅ is as defined in Scheme 8.

As set forth in Scheme 14, treatment of an amine compound of formula RR with an isocyanate in DCM at 40° C. will give a urea compound of the formula SS.

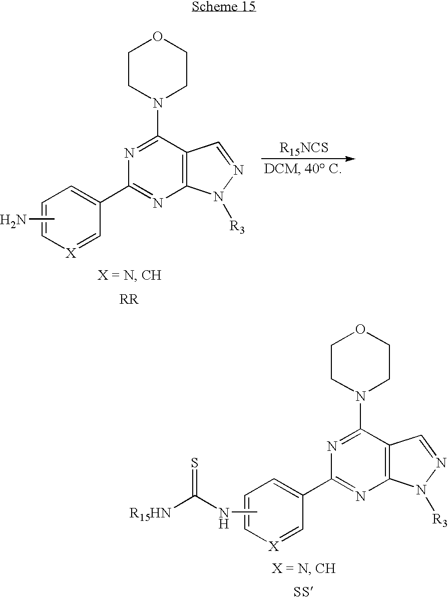

wherein R₃ is as defined above and R₁₅ is as defined in Scheme 8.

As set forth in Scheme 15, a compound of formula RR can be treated with an isothiocyanate in dichloromethane and excess triethylamine at 40° C. for 10 min. to give a thiourea compound of formula SS′.

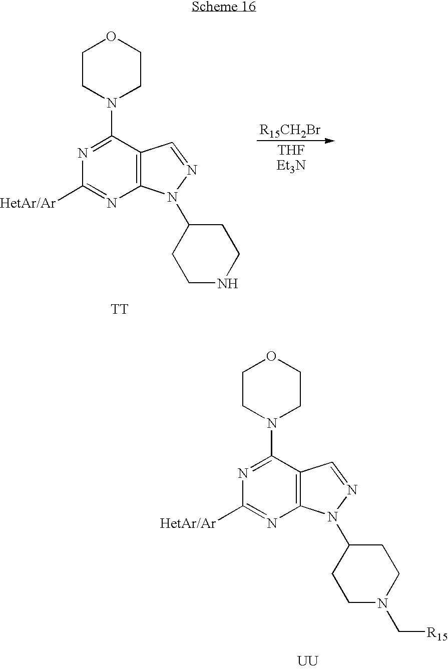

wherein R₁₅ is as defined in Scheme 8.

As set forth in Scheme 16, the piperidinyl nitrogen of a compound of formula TT can be alkylated with an alkyl bromide in THF with an excess of triethylamine to give a compound of formula UU.

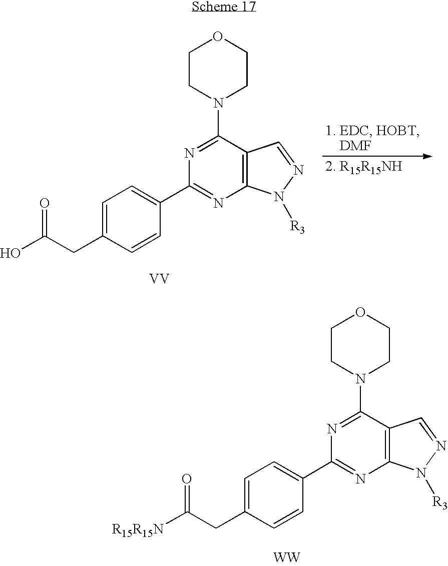

wherein R₃ is as defined above and R₁₅ is as defined in Scheme 8.

As set forth in Scheme 17, the carboxylic acid of a compound of formula VV can be amidated by reacting the acid with EDC in the presence of HOBT in DMF and then adding an amine by drops to the solution to give a compound of formula WW.

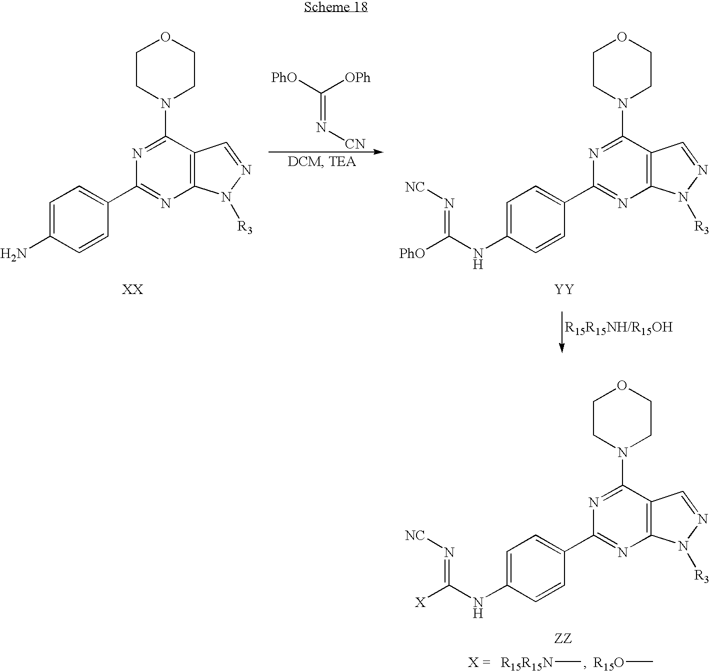

wherein R₃ is as defined above, and R₁₅ is as defined in Scheme 8.

As set forth in Scheme 18, an aniline compound of formula XX can be converted to a compound of the formula YY by treatment with diphenylcyanocarbonimidate in dichloromethane in the presence of triethylamine. Treatment of a compound of formula YY with an amine or an alcohol gave a compound of formula ZZ.

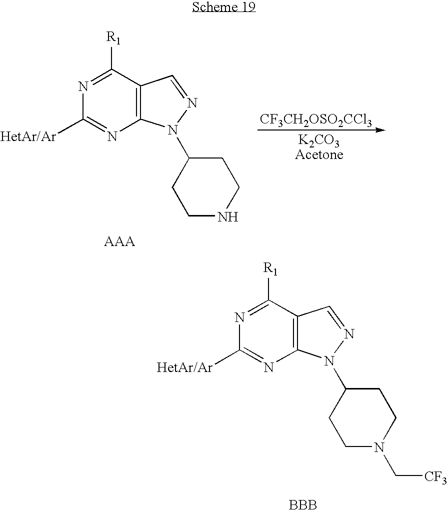

wherein R₁ is as defined above.

As set forth in Scheme 19 the amine of the compound of formula AAA was treated with 2,2,2-trifluoroethyl trichloromethanesulfonate in acetone in the presence of potassium carbonate to give a compound of the formula BBB.

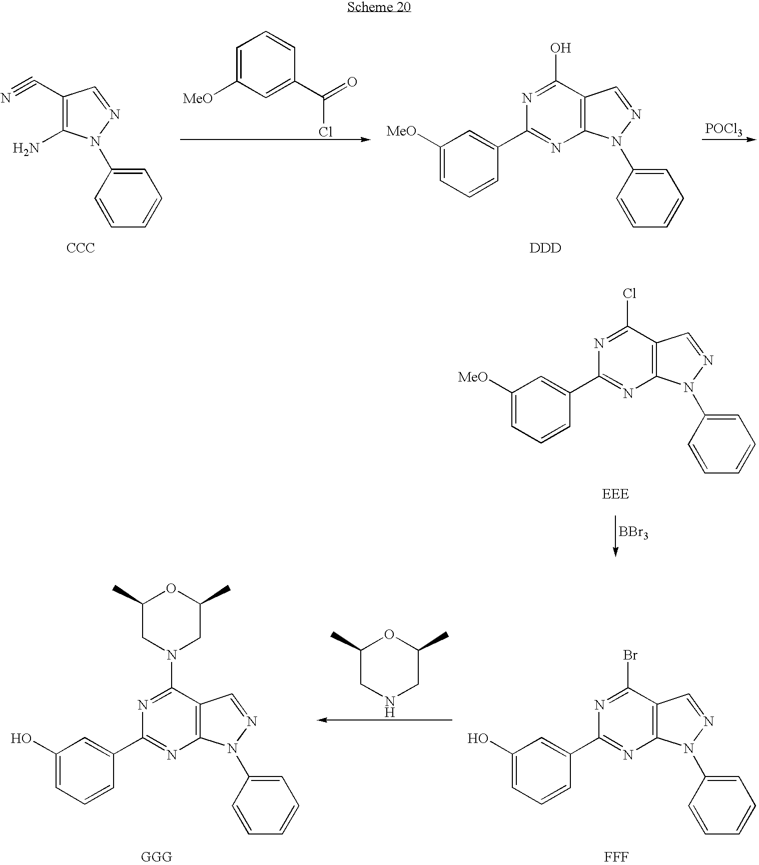

As set forth in Scheme 20, a compound of formula CCC can be treated with 3-methoxybenzoyl chloride to give a compound of the formula DDD. Treatment of a compound of formula DDD with phosphorus oxychloride will give a chloride compound of the formula EEE. Treatment of the compound of formula EEE with boron tribromide will give the bromide compound of formula FFF. Displacement of the bromine of a compound of formula FFF with cis-2, 6-dimethylmorpholine generates a compound of the formula GGG.

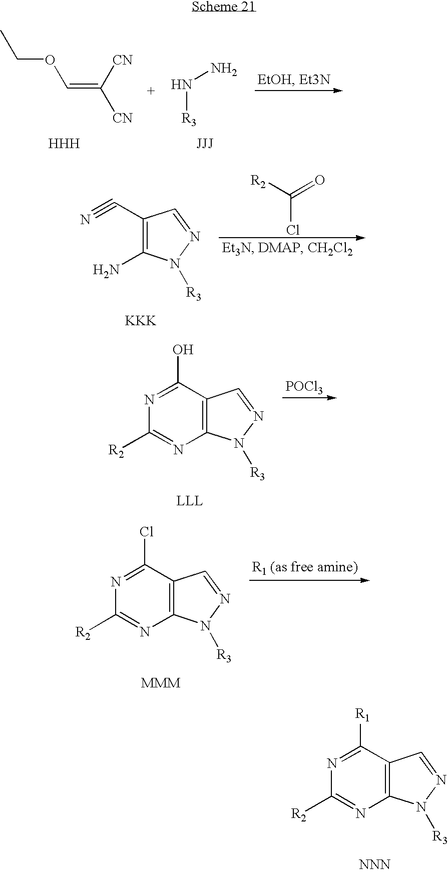

wherein R₁, R₂, and R₃ are as described above.

As set forth in Scheme 21, a compound of the formula HHH (ethoxymethylenemalononitrile) can be treated with hydrazine JJJ in ethanol in the presence of triethylamine to give compound KKK. Treatment of a compound of the formula KKK with an acid chloride in dichloromethane in the presence of DMAP and triethylamine will produce a compound of formula LLL. Treatment of the compound of formula LLL with phosphorous oxychloride will give the chloride compound of formula MMM. Subsequent treatment of a compound of the formula MMM with an amine gives a compound of the formula NNN.

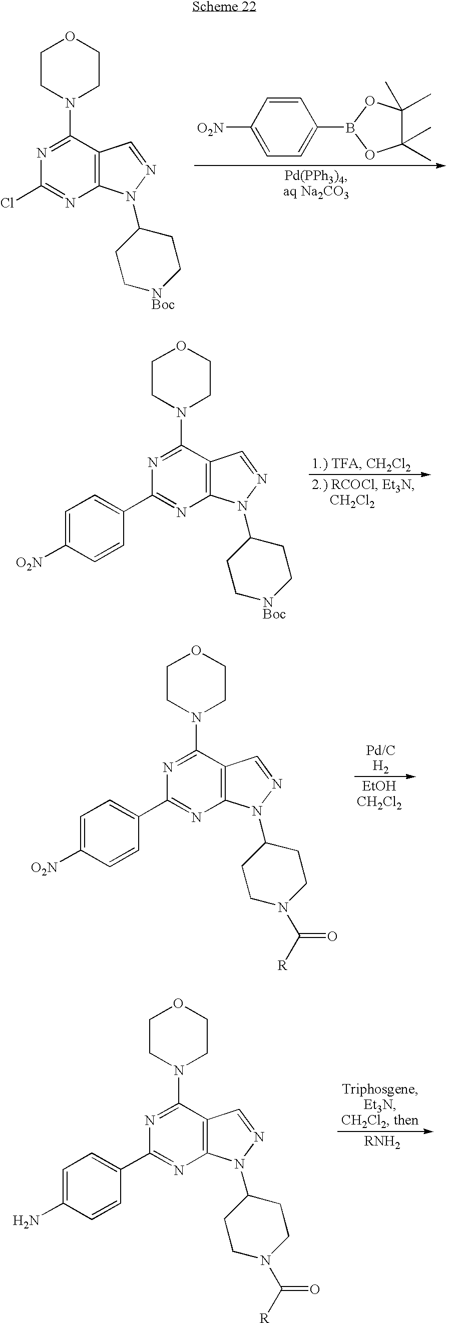

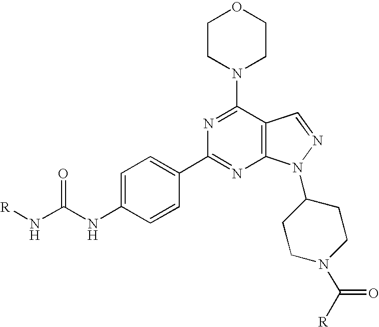

As set forth in Scheme 22, compounds with R₂ substituted by —NHC(O)NR¹⁶R¹⁷, the urea functionality can be assembled as shown.

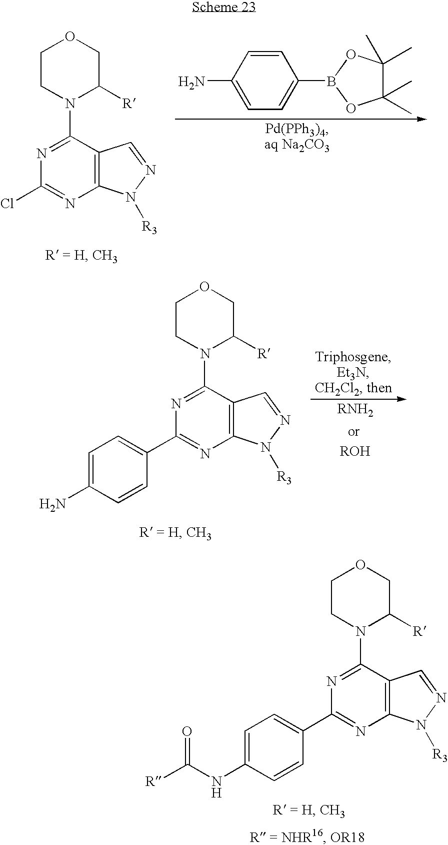

As set forth in Scheme 23, when R₂ is substituted by —NHC(O)NR¹⁶R¹⁷ or —NHC(O)OR¹⁸, then chemistry similar to that used in Scheme 22 can be used.

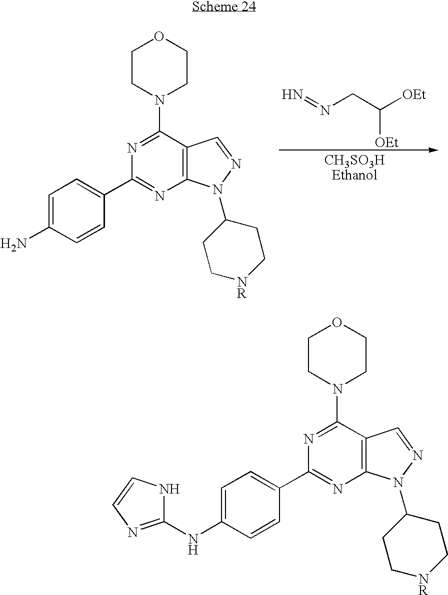

As set forth in Scheme 24, compounds with R₂ substituted by NR¹⁶R¹⁷ and R¹⁶ is 2-imidazoyl, then the imidazole ring can be assembled in place.

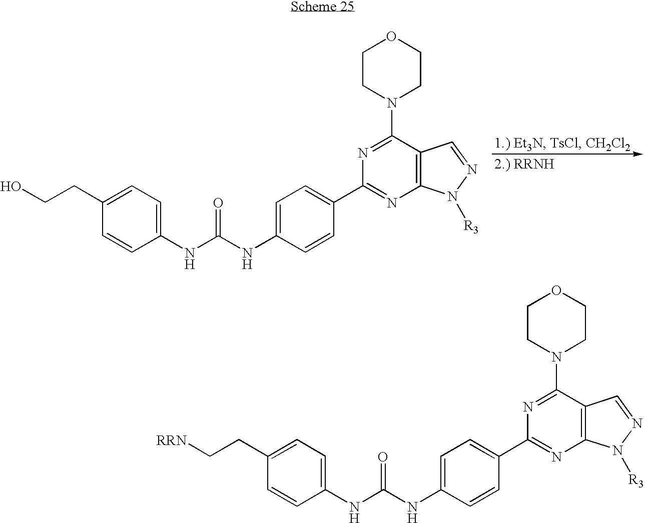

As set forth in Scheme 25, compounds with R₂ substituted by —NHC(O)NR¹⁶R¹⁷ and R₁₆ is C₁-C₆alkyl, substituted by amino can be made by tosylate displacement.

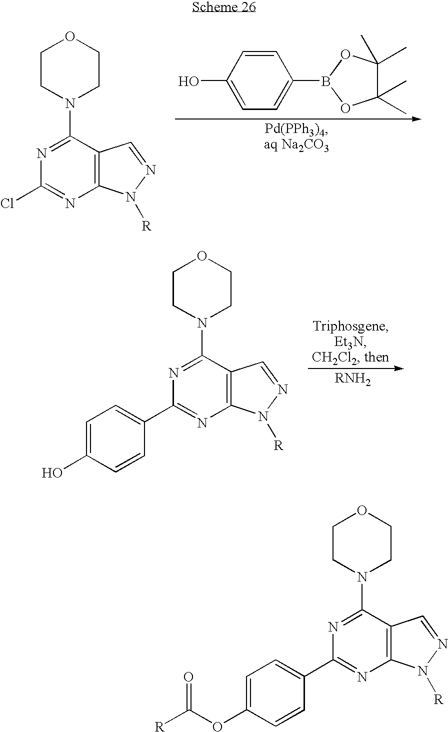

As set forth in Scheme 26, when R₂ is substituted by —O—C(O)NR¹⁶R¹⁷, then the carbamate can be prepared from a phenol.

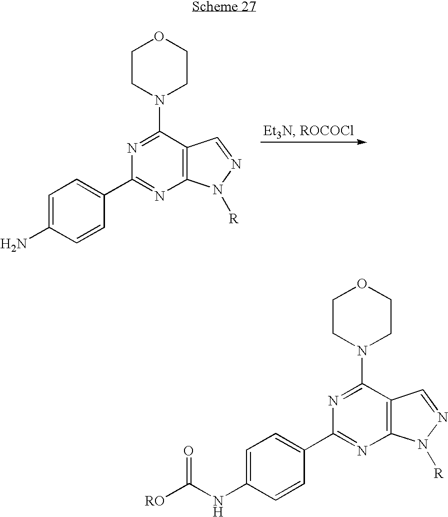

As set forth in Scheme 27, when R₂ is substituted by —NHC(O)OR¹⁸, then chemistry similar to that used in Scheme 23 can be used to make the carbamate from the aniline.

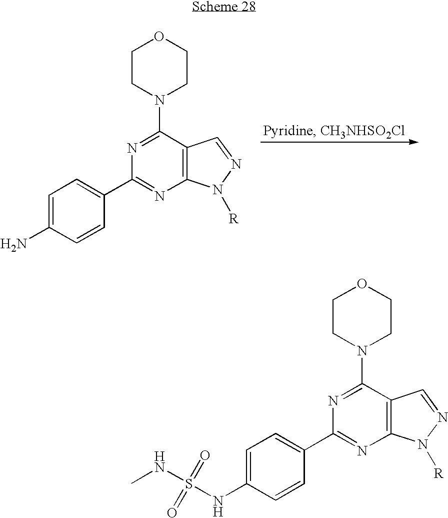

As set forth in Scheme 28, the aniline can also be used to make the —NH(SO₂)NH—(C₁-C₆alkyl) functionality.

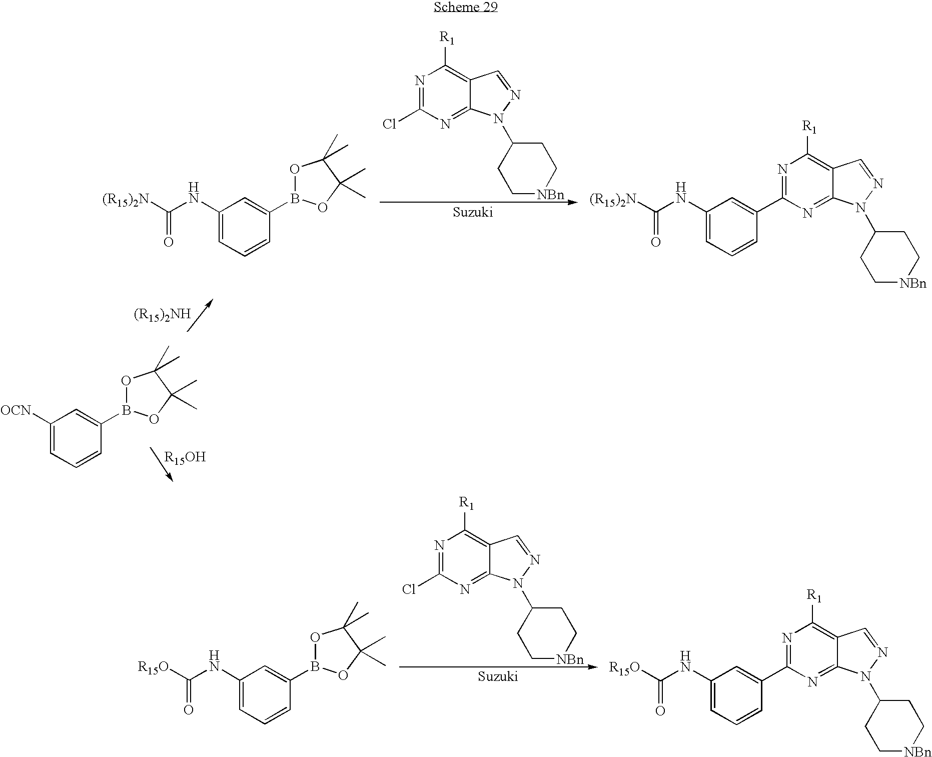

As set forth in Scheme 29, the Suzuki coupling can also be preformed on R₂═C₆-C₁₄aryl groups with —NHC(O)NR₁₆R¹⁷ or —NHC(O)OR¹⁸ substituents in place.

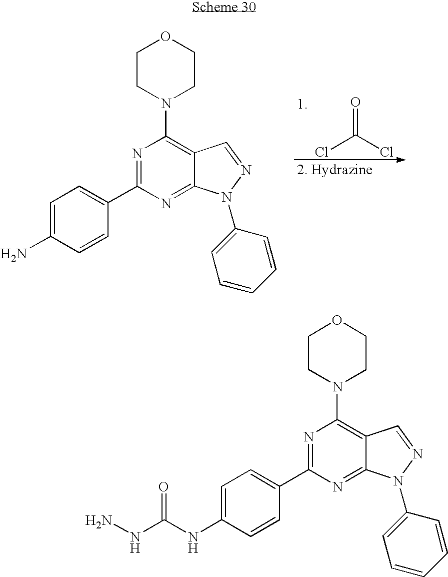

As set forth in Scheme 30, with the R₂ group substituted by —NHC(O)NHNR¹⁶R¹⁷ and R¹⁶═R¹⁷=hydrogen, the NHNR¹⁶R¹⁷ group is made from hydrazine.

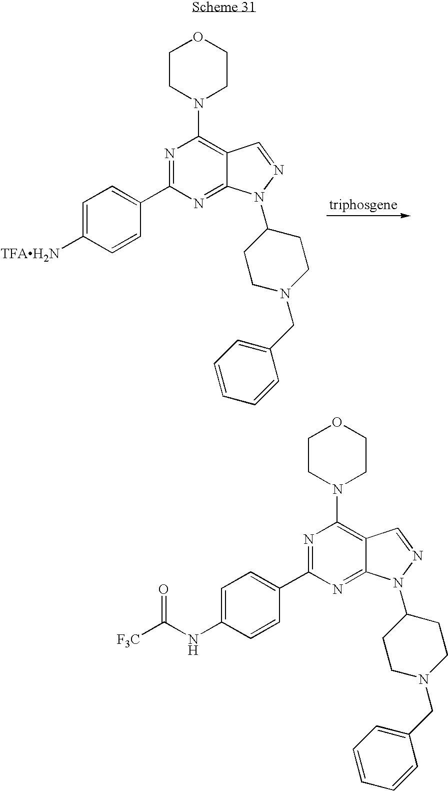

As set forth in Scheme 31, compounds with R¹⁸═C₁-C₆perfluoroalkyl ═CF₃ can be made from the trifluoroacetate salt as the source of the C₁-C₆perfluoroalkyl radical.

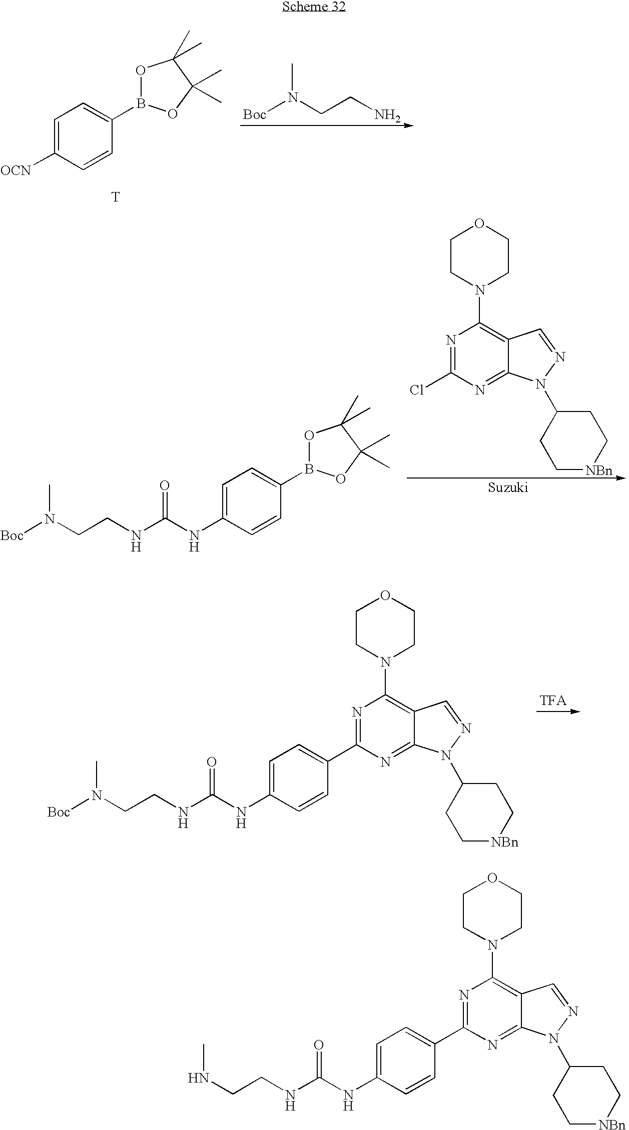

As set forth in Scheme 32, the compounds with the R₂ group substituted by —NHC(O)NR¹⁶R¹⁷ and R¹⁶═C₁-C₆alkyl substituted by amino can be made with Suzuki coupling on a protected amine-containing boronic acid.

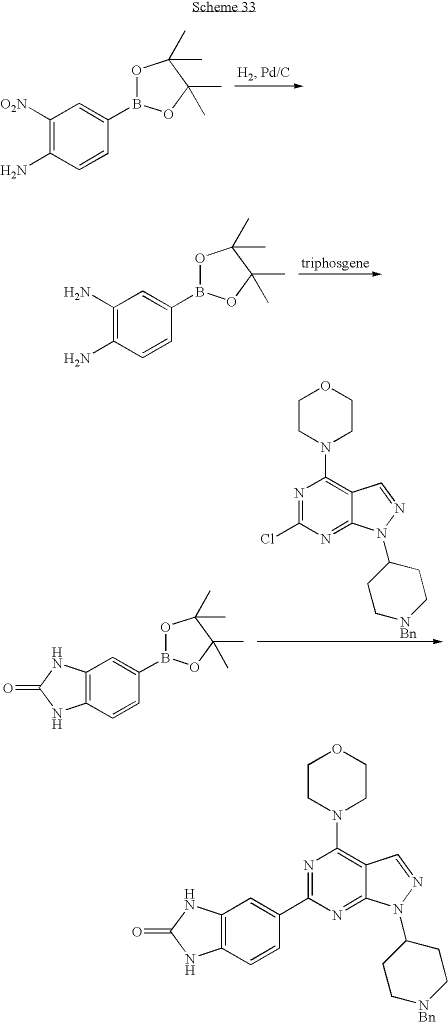

As set forth in Scheme 33, a compound with R₂=1H-benzo[d]imidazol-6-yl-2-ol is readily made with the appropriate boronic acid. The compound is shown above in its tautomeric keto form.

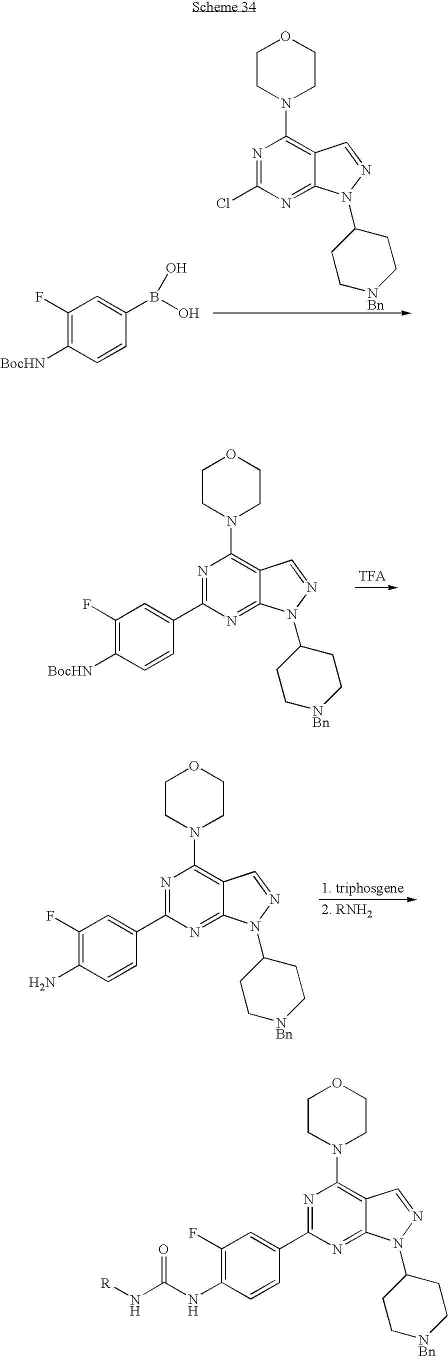

As set forth in Scheme 34, a compound with R═R¹⁶ and a fluorine atom on the R₂=aryl ring can be made with a protected boronic acid.

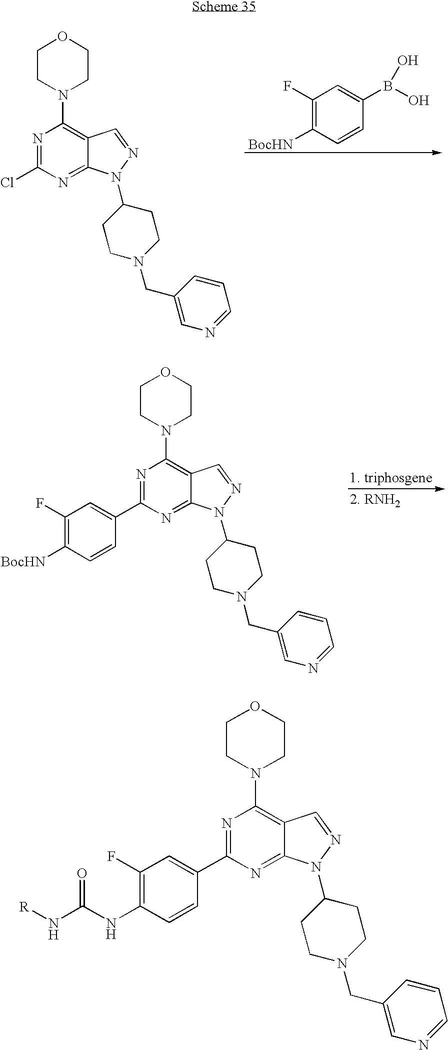

As set forth in Scheme 35, compounds with R₃=monocyclic C₁-C₆heterocycle substituted by heteroaryl(C₁-C₆alkyl) can be made by a process similar to that shown in Scheme 34.

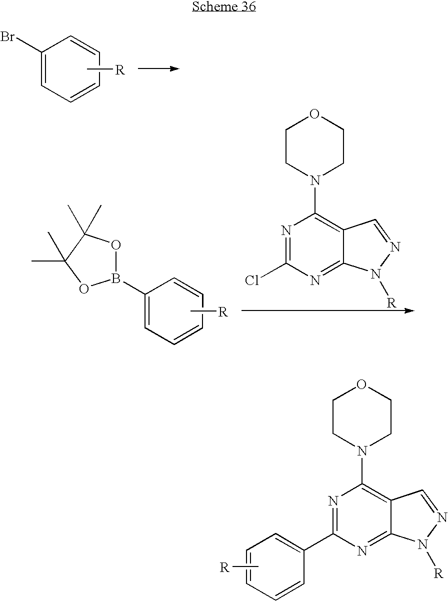

As set forth in Scheme 36, the boronic esters needed for Suzuki coupling are easily obtained from the C₁-C₁₄aryl bromide precursors.

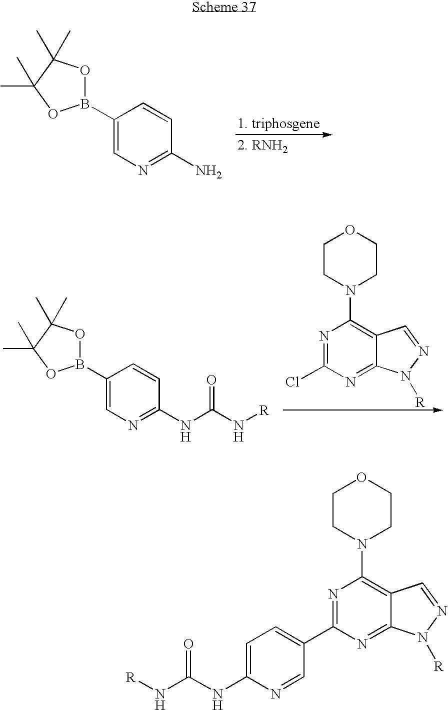

As set forth in Scheme 37, a compound with R₂═C₁-C₉heteroaryl groups and a —NHC(O)NR¹⁶R¹⁷ substituent in place can be made by Suzuki coupling with the appropriate 4-(6-chloro-1H-pyrazolo[3,4-d]pyrimidin-4-yl)morpholine compound.

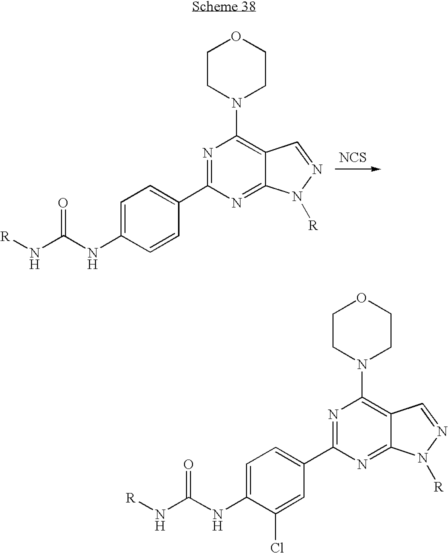

As set forth in Scheme 38, compounds with R₂═C₆-C₁₄aryl groups and a —NHC(O)NR¹⁶R¹⁷ substituent in place can be further substituted on the C₆-C₁₄aryl ring by free-radical chlorination.

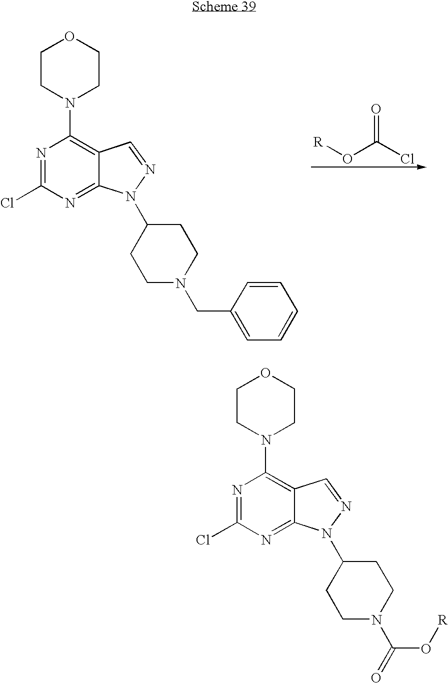

As set forth in Scheme 39, intermediates useful for making compounds with R₃=monocyclic C₁-C₆heterocycle substituted with (C₁-C₆alkoxy)carbonyl can be made by removal of a benzyl protecting group.

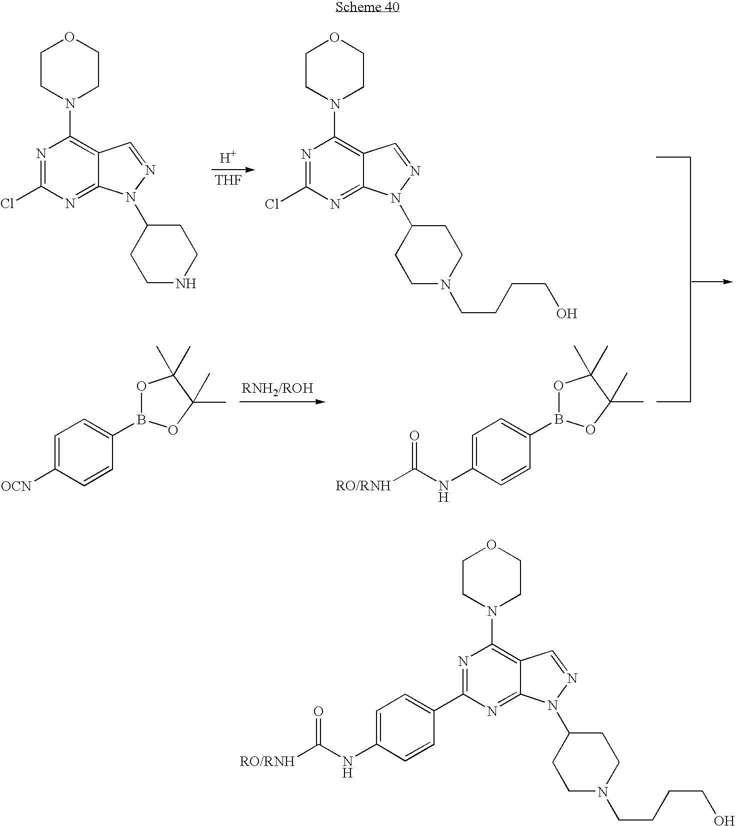

As set forth in Scheme 40, Suzuki coupling can be performed on a 4-(6-chloro-1H-pyrazolo[3,4-d]pyrimidin-4-yl)morpholine compound bearing a C₁-C₆hydroxylalkyl group on the R₃ radical without protection of the hydroxyl functionality.

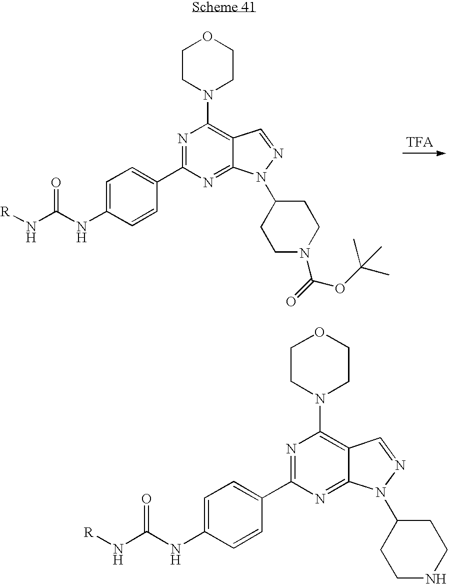

As set forth in Scheme 41, cleavage of t-BOC groups was done by conventional means.

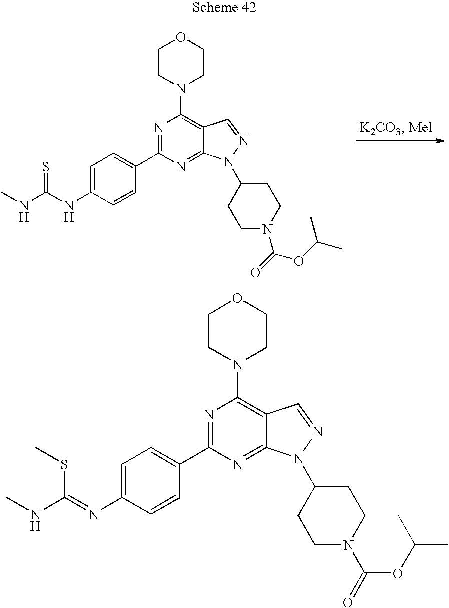

As set forth in Scheme 42, thioamides were smoothly converted into —N═C(S—C₁-C₆alkyl)(NH—C₁-C₆alkyl) groups.

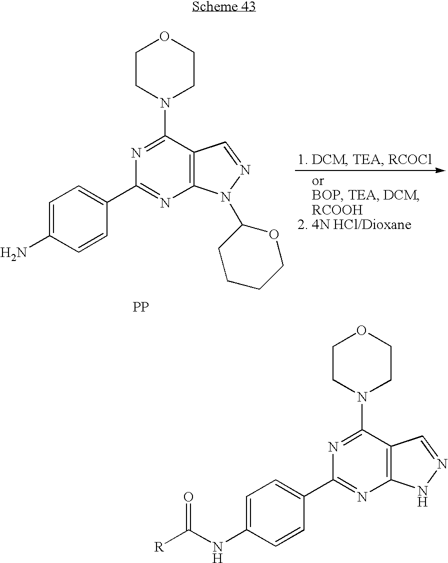

As set forth in Scheme 43, acylation of the aniline PP preceeded removal of the tetrahydro-2H-pyran-2-yl group from position 1 of the 1H-pyrazolo[3,4-d]pyrimidine ring.

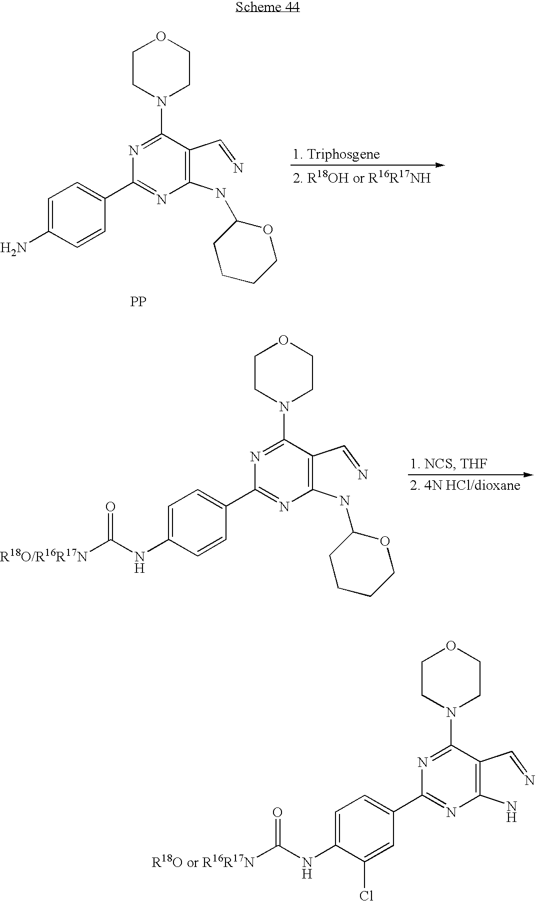

As set forth in Scheme 44, free radical chlorination, like that shown in Scheme 38, preceeded removal of the THP protecting group.

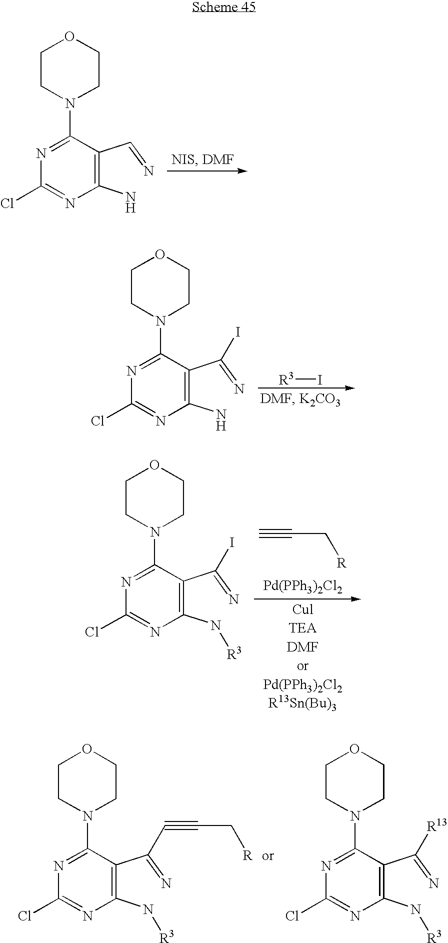

As set forth in Scheme 45, a compound with R₁₃=substituted C₂-C₆alkynyl were made from the 3-iodo-1H-pyrazolo[3,4-d]pyrimidine compounds.

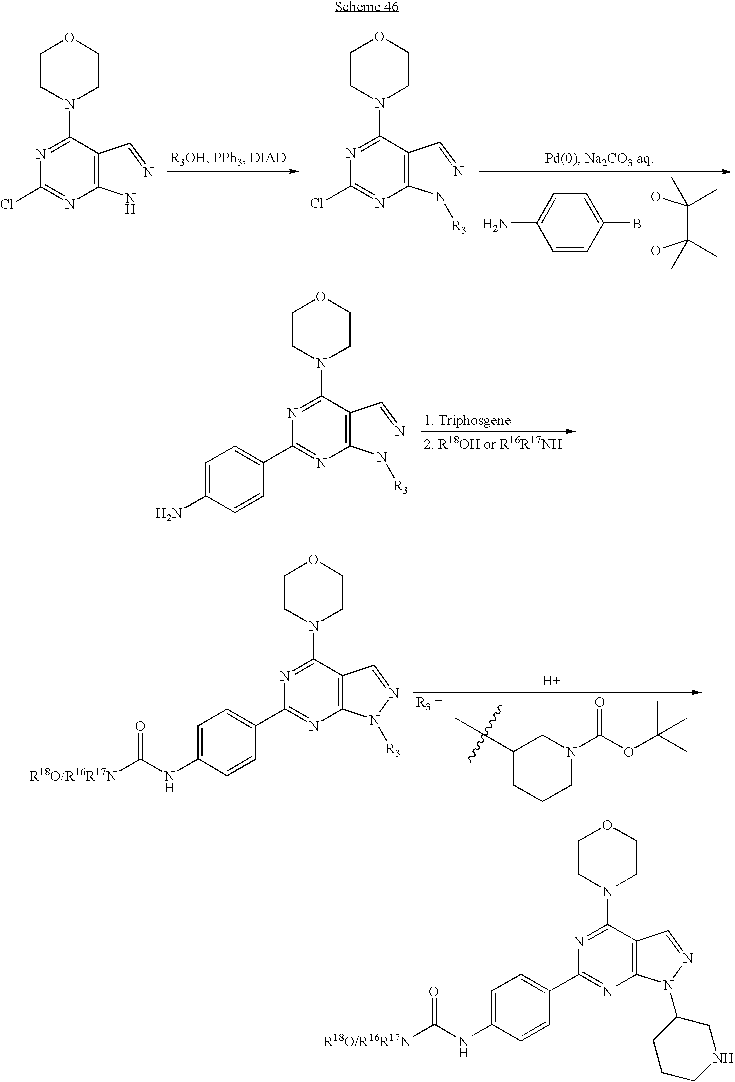

As set forth in Scheme 46, a compound with R₃=piperidin-3-yl was made from a BOC-protected piperidin-3-ol starting material.

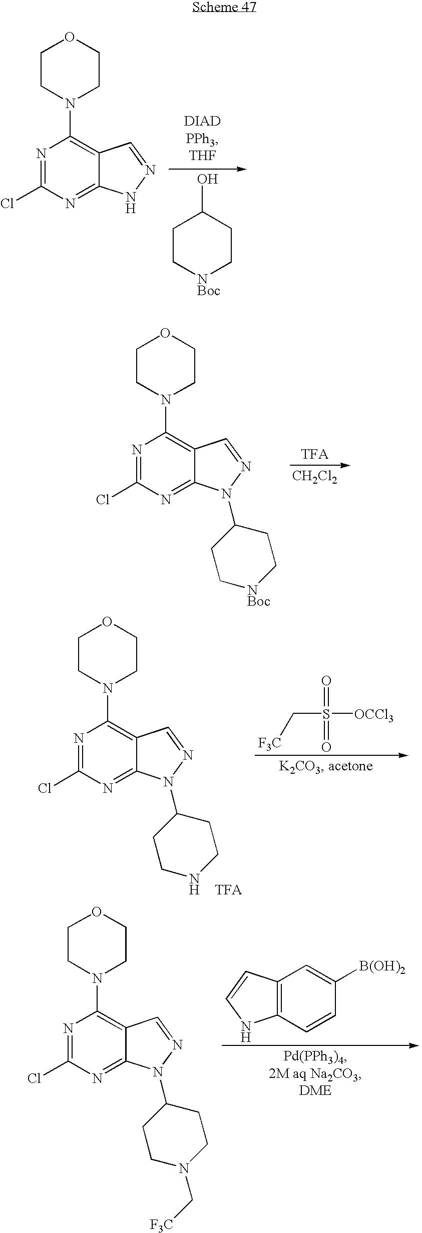

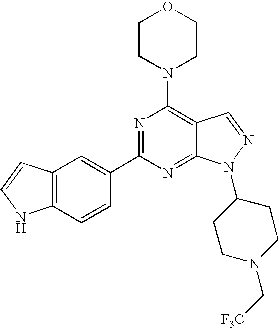

As set forth in Scheme 47, a compound with R₂=5-indolyl was made with the appropriate boronic acid.

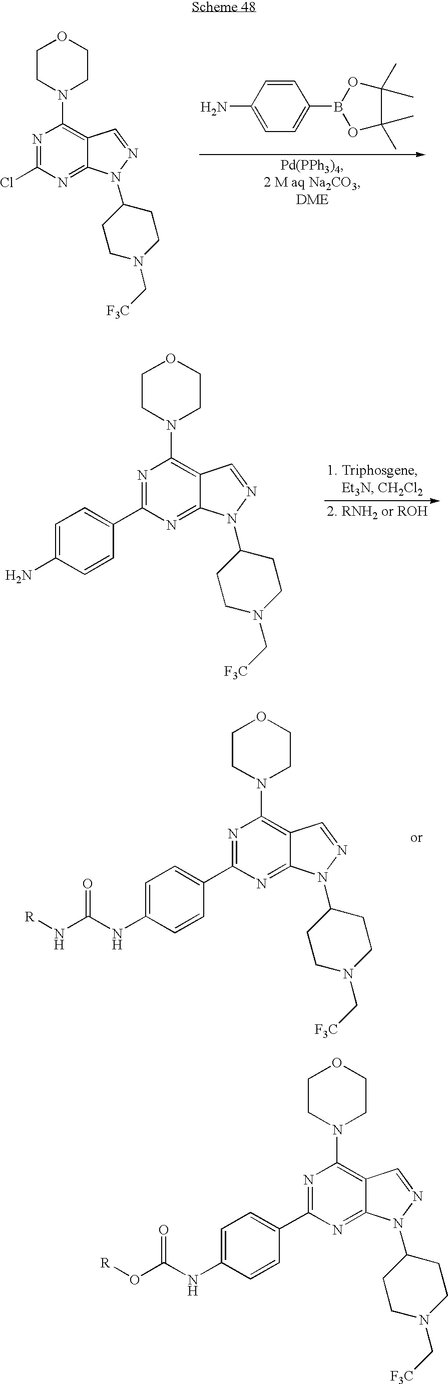

As set forth in Scheme 48, compounds with R₃=monocyclic C₁-C₆heterocycle optionally substituted with C₁-C₆perfluoroalkyl were made as illustrated.

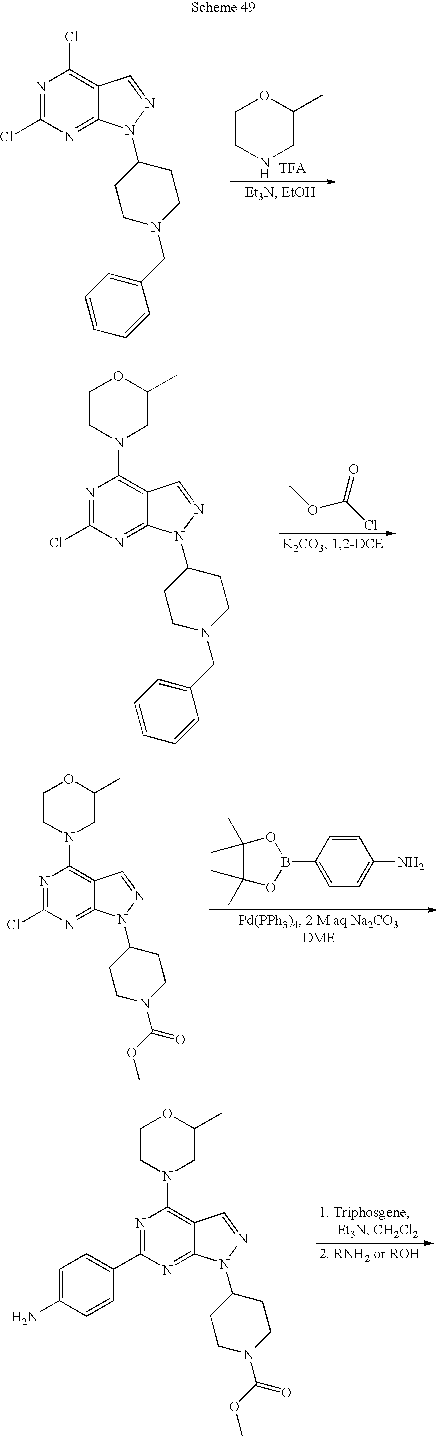

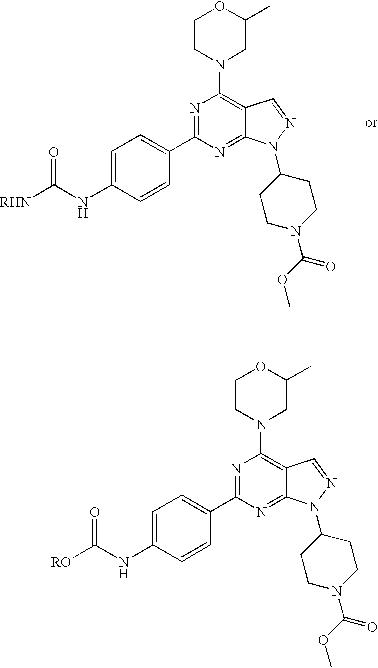

As set forth in Scheme 49, compounds with R₁ substituted by a methyl group on position 2 of the morpholine ring were made by displacement of the chlorine atom at position 4 of 1-(1-benzylpiperidin-4-yl)-4,6-dichloro-1H-pyrazolo[3,4-d]pyrimidine.

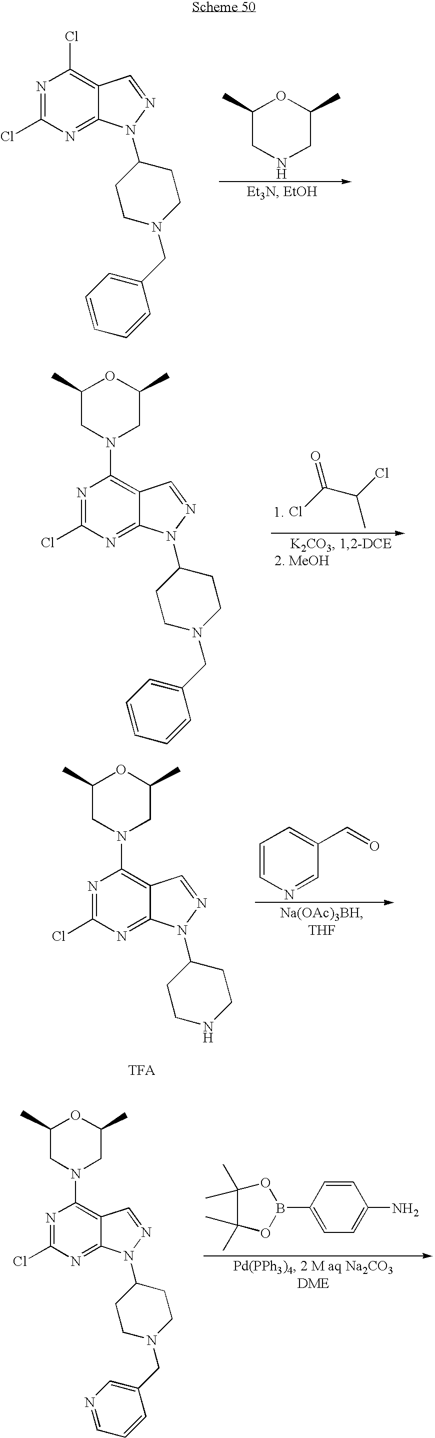

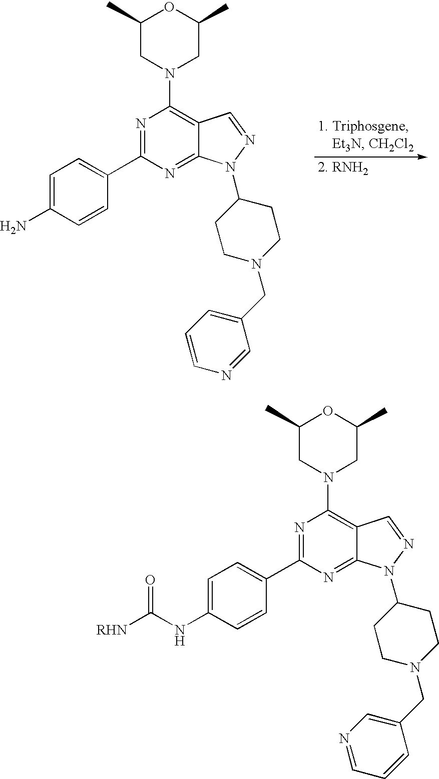

As set forth in Scheme 50, removal of the benzyl protecting group followed by reductive amination gave a 6-chloro-1H-pyrazolo[3,4-d]pyrimidine, which was subjected to Suzuki coupling.

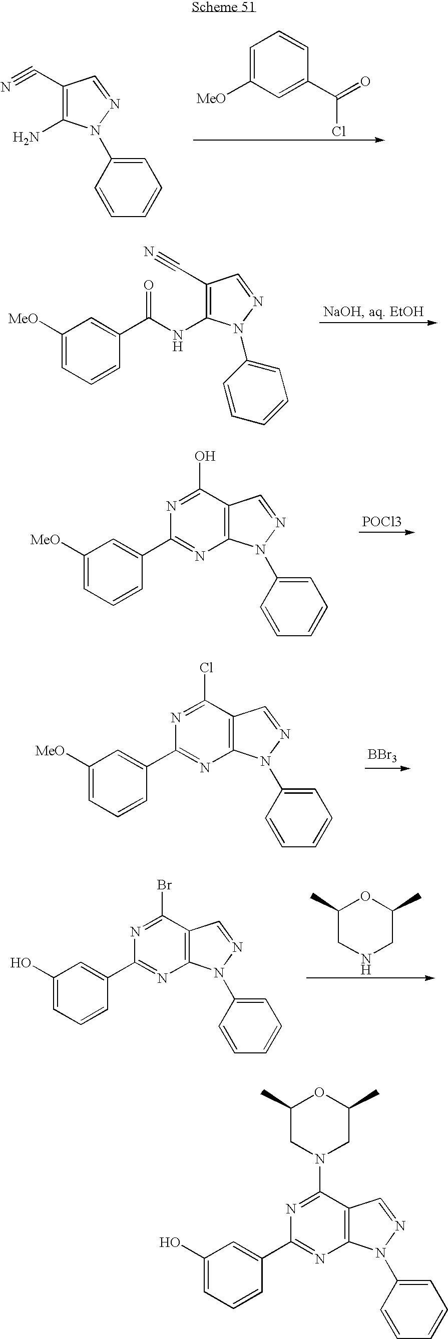

As set forth in Scheme 51, the pyrimidine ring of the 1H-pyrazolo[3,4-d]pyrimidine compounds could be anulated to the pyrazole precursor.

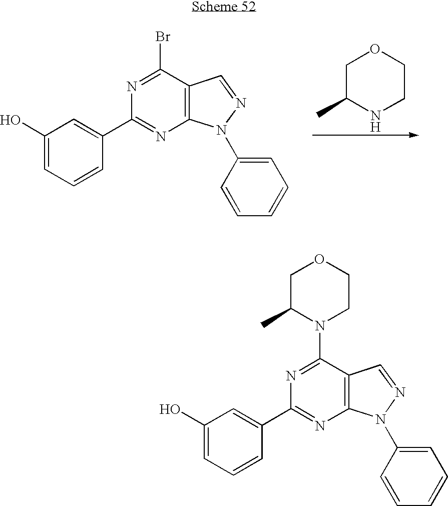

As set forth in Scheme 52, a compound, in resolved form and bearing a methyl group on position 3 of the R₁=morpholine ring was made in one step from the appropriate precursor.

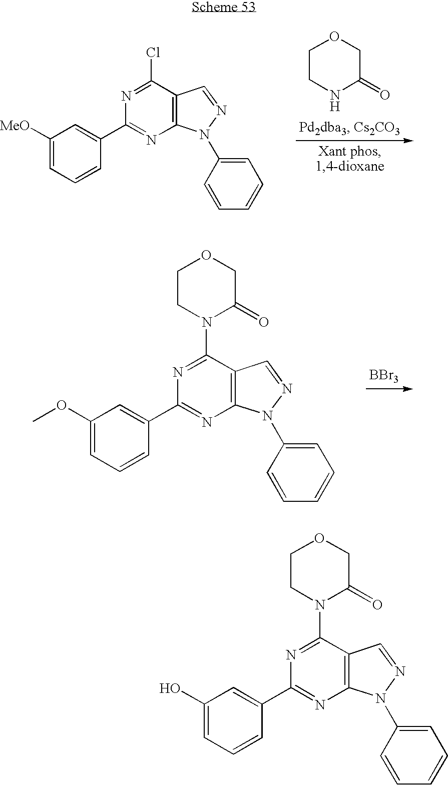

As set forth in Scheme 53, palladium catalyzed coupling of morpholin-3-one with 4-chloro-6-(3-methoxyphenyl)-1-phenyl-1H-pyrazolo[3,4-d]pyrimidine gave a compound with R₁ containing a carbonyl (C═O) group.

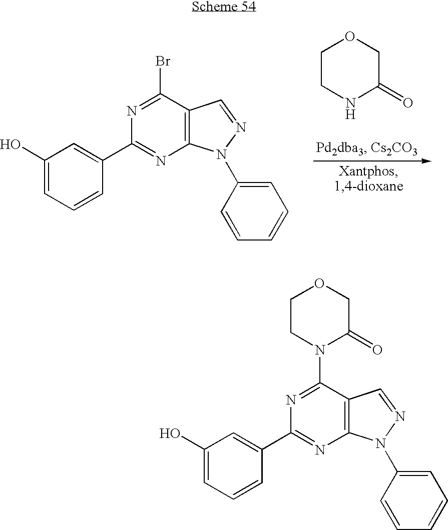

As set forth in Scheme 54, the palladium catalyzed coupling of morpholin-3-one could be done without phenolic protection.

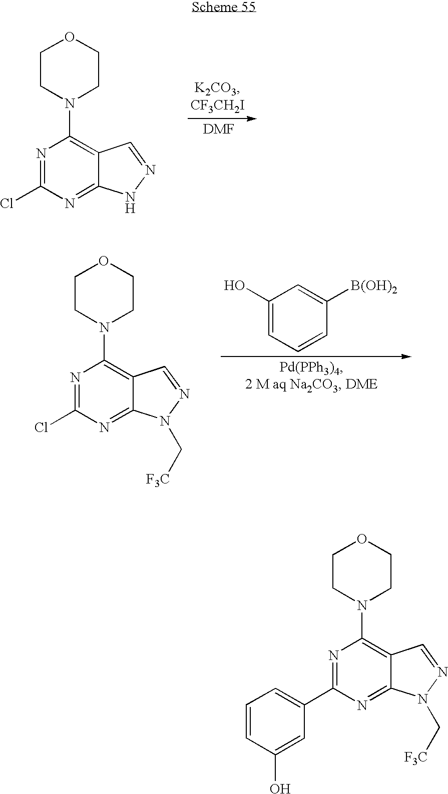

As set forth in Scheme 55, a boronic acid bearing an unprotected phenolic group underwent smooth Suzuki coupling.

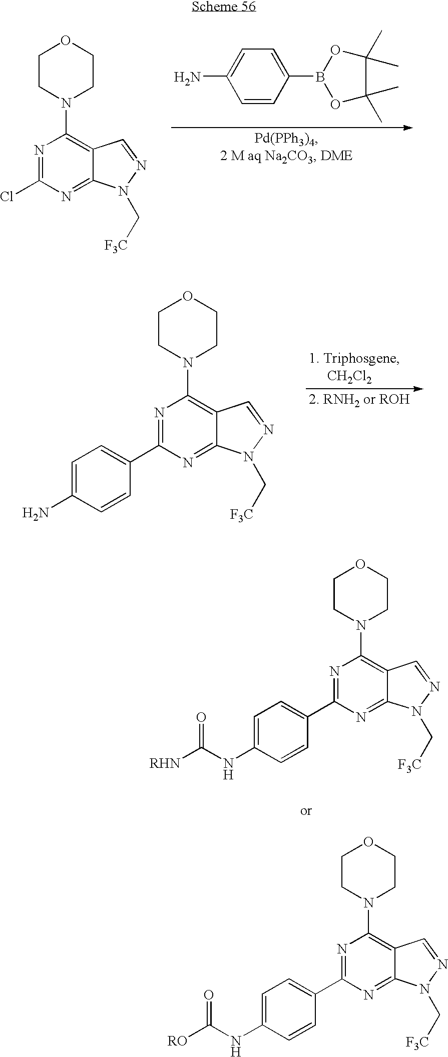

As set forth in Scheme 56, compounds with R₃═C₁-C₆perfluoroalkyl are readily made.

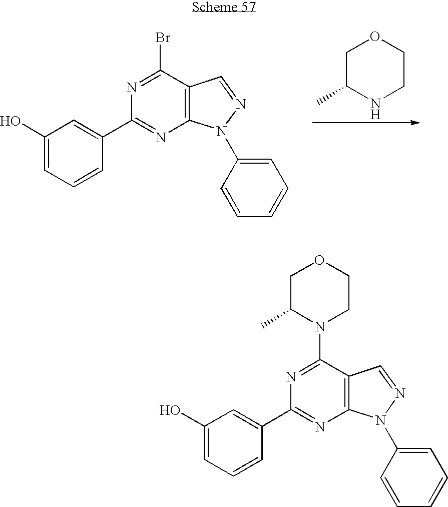

As set forth in Scheme 57, a compound with opposite absolute stereochemistry was made with a procedure outlined in Scheme 52.

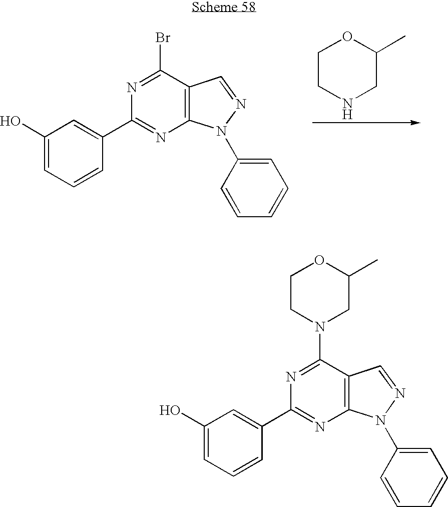

As set forth in Scheme 58, a compound with R₁ substituted by a methyl group on position 2 of the morpholine ring were made by displacement of the bromine atom at position 4 of the 1H-pyrazolo[3,4-d]pyrimidine bearing a free phenolic substituent.

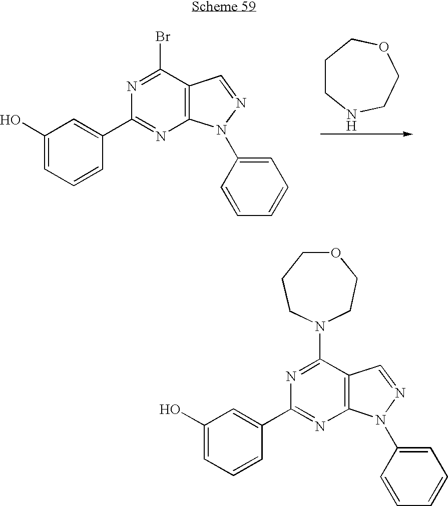

As set forth in Scheme 59, a compound with R₁=homomorpholine was prepared.

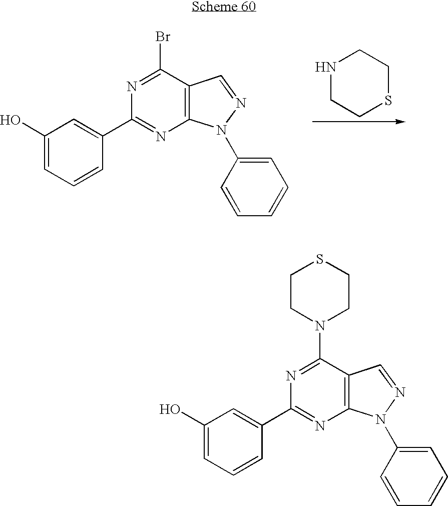

As set forth in Scheme 60, a compound with R₁=thiomorpholine was also prepared.

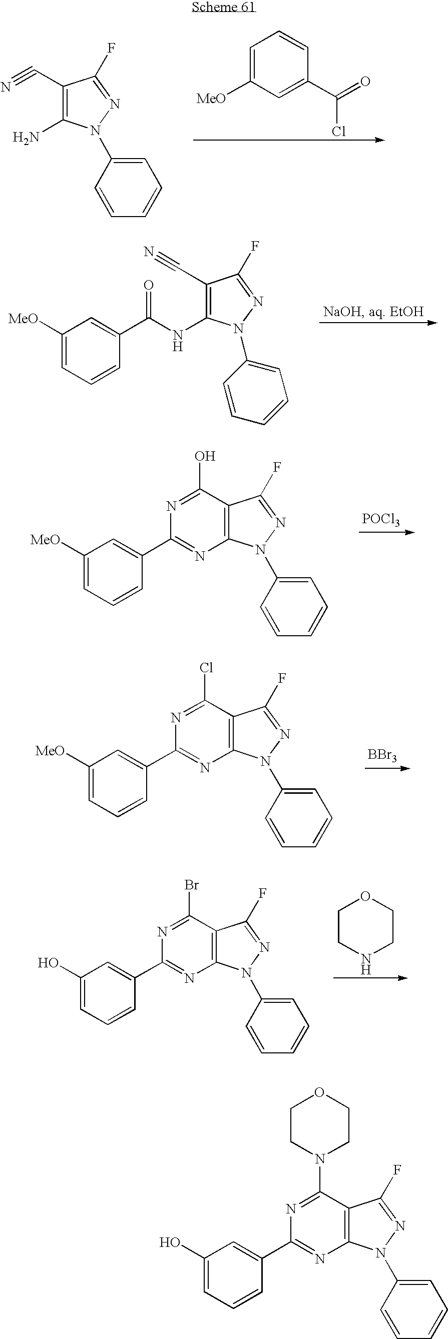

As set forth in Scheme 61, a compound with R₁₃=the halogen fluorine was made by anulating the pyrimidine ring of the 1H-pyrazolo[3,4-d]pyrimidine to the pyrazole precursor by a process similar to that shown in Scheme 51.

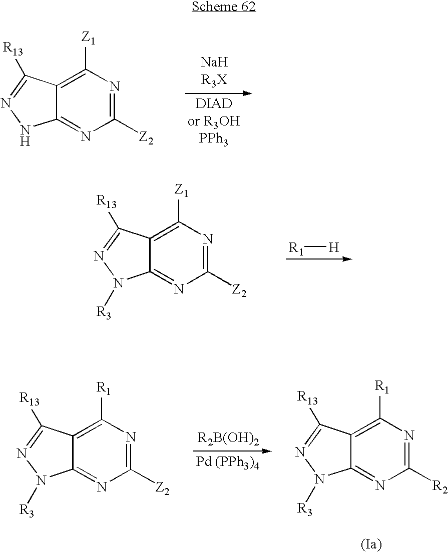

wherein Z₁, and Z₂ are each independently halogen and R₁-R₃ and R₁₃ are as defined above in Formula Ia.

The synthesis of the desired 1H-pyrazolo[3,4-d]pyrimidine analogs of Formula (Ia) may be prepared according to Scheme 62 by first reacting the available 4,6-dihalo-3-substituted-1H-pyrazolo[3,4-d]pyrimidine shown above with alcohols R₃OH under standard Mitsunobu reaction conditions or by standard alkylation with R₃—X where X is a leaving group. Reaction with amine R₁—H followed by Suzuki reaction with boronic acids R₂B(OH)₂ under either microwave or thermal conditions gives product Ia. The boronic acids are commercially available or can be prepared synthetically via standard organic chemistry protocols. The starting material 4,6-dihalo-3-substituted-1H-pyrazolo[3,4-d]pyrimidine in Scheme 62 was obtained from either commercial sources or prepared by well-known literature procedures

The general procedures used to synthesize the compounds of Formula 1a are described in Reaction Schemes 1-62 and are illustrated in the examples. Reasonable variations of the described procedures, which would be evident to one skilled in the art, are intended to be within the scope of the present invention.

The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials.

### EXAMPLES

### General Motors

The following general methods outline the synthesis the Pyrazolopyrimidine Analogs of the Examples.

General: Preparatory HPLC using a Gilson Instrument

Crude material is dissolved in 1.5 ml DMSO and 0.5 ml MeOH, filtered through a 0.45 μm GMF, and purified on a Gilson HPLC, using a Phenomenex LUNA C₁₈ column: 60 mm×21.20 mm I.D., 5 μm particle size, with ACN/water (containing 0.2% TFA or ammonium hydroxide) gradient elution. The appropriate fractions are then analyzed by LC/MS.

Analytical HPLC Conditions: Instrument—Agilent 1100; Column: Thermo Aquasil C18, 50×2.1 mm, 5 μm; Mobile Phase: A: 0.1% Formic Acid in water; B: 0.1% Formic Acid in ACN; Flow Rate: 0.800 mL/min.; Column Temperature: 40° C.; Injection Volume: 5 mL; UV: monitor 215, 230, 254, 280, and 300 nm; Purity is reported at 254 nm unless otherwise noted.

Gradient Table:

| Time (min) | % B |
|---|---|
| 0 | 5 |
| 2.5 | 95 |
| 4.0 | 95 |
| 4.1 | 5 |
| 5.5 | 5 |

MS Conditions: Instrument: Agilent MSD; Ionization Mode: API-ES; Gas Temperature: 350° C.; Drying Gas: 11.0 L/min.; Nebulizer Pressure: 55psig; Polarity: 50% positive, 50% negative; VCap: 3000V (positive), 2500V (negative); Fragmentor: 80 (positive), 120 (negative); Mass Range: 100-1000 m/z; Threshold: 150; Step size: 0.15; Gain: 1; Peak width: 0.15 min.

### Example 1

### 2,4,6-Trichloro-pyrimidine-5-carbaldehyde (Scheme 1)

To a solution of POCl₃ (200 mL) in DMF (42 mL) cooled to 0° C. is slowly added barbituric acid (30 g) over 1.5 hrs. The mixture is then heated to reflux for 16 hrs and then evaporated (the distillate is carefully decomposed by slowly pouring into stirred ice methanol slush). The remainder is cooled to 0° C. and added very slowly to a solution of ice water upon which a beige solid forms. The solid is filtered, dissolved in DCM, washed with water, washed with a sat. NaHCO₃ solution, dried (MgSO₄), and concentrated in vacuo to give white crystals (24 g).

### Example 2

### 4,6-Dichloro-1-methyl-1H-pyrazolo[3,4-d]pyrimidine (Scheme 1)

To a solution of the chloroaldehyde (3.7 g, 17.5 mmol) dissolved in EtOH (50 mL) and cooled to 78° C. is added methyl hydrazine (0.93 mL, 17.5 mmol) and TEA (8 mL). The mixture is stirred for 30 min. at −78° C. then 2 hr at 0° C. The solution is then concentrated in vacuo without heating. To the reduced volume solution is added EtOAc and the solution washed with a sat. NaHCO₃ solution and concentrated in vacuo without heating. Filtration over a small silica gel plug (2:1 EtOAc:Hex) and concentration affords the desired product as a yellow solid.

### Example 3

### 4,6-Dichloro-1-(1-ethyl-piperidin-4-yl)-1H-pyrazolo[3,4-d]pyrimidine (Scheme 1)

To a solution of the chloroaldehyde (2.5 g, 11.6 mmol) dissolved in EtOH (40 mL) and cooled to 78° C. is added N-benzyl-4-piperidinzyl-hydrazine dihydrochloride (3.3 g, 11.6 mmol) and TEA (5 mL). The mixture is stirred for 30 min. at −78° C. then 2 hr at 0° C. The solution is then concentrated in vacuo without heating. To the reduced volume solution, EtOAc and a sat. NaHCO₃ solution is added and the solution filtered over diatomaceous earth and separated. The organic layer is dried (MgSO₄) and concentrated in vacuo without heating. Filtration over a small silica gel plug (EtOAc) and concentration affords the desired product as a yellow solid (3 g).

### Example 4

### 3-[1-(1-Benzyl-piperidin-4-yl)-4-morpholin-4-yl-1H-pyrazolo[3,4-d]pyrimidin-6-yl]-phenol (Scheme 2)

To a solution of the dichloride (6.20 g, 17.12 mmol) dissolved in EtOH (100 mL) is added morpholine (1.5 mL, 17.12 mmol) and the reaction stirred overnight. The solvent is then evaporated and the remainder triturated with diethyl ether/hexane. The yellow solid is filtered off and washed with hexane. Drying on the fritted funnel provides yellow amorphous solid (5.25 g).

The above prepared monochloride (2.13 g) and m-hydroxybenzylboronic acid (1.0 g) are dissolved in dioxane (50 mL). Sodium carbonate (2.0 M solution in water) 10 mL is added followed by tetrakistriphenylphosphine palladium (0) (50 mg). The solution is deoxygenated (3 cycles of vacuum/nitrogen) and heated to 100° C. overnight. The solution is then evaporated and the residue treated with water (50 mL). The pH is adjusted to 7 and the solution extracted with ethyl acetate. After application onto a pad of silica, the product is eluted with 20% methanol in ethyl acetate giving a yellow solid (3 g). Small-scale reactions (50 mg scale) of this type are run in microwave (160° C., 5 min). The crude reaction is concentrated and purified via preparatory HPLC using a Gilson instrument.

### Example 5

### 3-(4-Morpholin-4-yl-1-piperidin-4-yl-1H-pyrazolo[3,4-d]pyrimidin-6-yl)-phenol (Scheme 3)

3-[1-(1-Benzyl-piperidin-4-yl)-4-morpholin-4-yl-1H-pyrazolo[3,4-d]pyrimidin-6-yl]-phenol (0.25 g) is dissolved in methanol (20 mL). 10% palladium hydroxide on carbon (25 mg) is added and the solution hydrogenated at atmospheric pressure. The end point is determined by LCMS (ca.3 h). The catalyst is then filtered away through a diatomaceous earth pad and the solvent evaporated leaving a white solid (148 mg).

### Example 6

### N-Substituted-3-(4-morpholin-4-yl-1-piperidin-4-yl-1H-pyrazolo[3,4-d]pyrimidin-6-yl)-phenol (Scheme 3)

### Example 6A

### Amides

The piperidine compound (70 mg) is dissolved in tetrahydrofuran (5 mL), afterwards triethyl amine (0.1 mL) is added followed by acid chloride (1.0 eq). The solution is then stirred for 1 hour and evaporated. The residue is purified via preparatory HPLC using a Gilson instrument.

### Example 6B

### Amines

The piperidine compound (50 mg) is dissolved in methanol (5 mL). An aldehyde (3 eq) is added followed by sodium cyanoborohydride (20 mg) and acetic acid (25 μL), and the solution stirred overnight. The solution is then evaporated, neutralized with 1.0 M HCl and partitioned between ethyl acetate and sodium bicarbonate. The organic phase is then separated, evaporated and the residue purified via preparatory HPLC using a Gilson instrument.

### Example 7

### (1-Benzyl-piperidin-4-yl)-hydrazine dihydrochloride

Benzoic hydrazide (27 g) is dissolved in methanol (150 mL). 1-Benzyl-piperidin-4-one (37.8 g) is added and the solution heated at 30° C. for 1 h and 60° C. for a further 2 h. The solution is then cooled to 0° C. and sodium borohydride (6.8 g) added in portions. After 2 h, the solution is evaporated and the residue was partitioned between dichloromethane and water. The organic phase is then dried with anhydrous magnesium sulfate and evaporated leaving an oil (102 g).

The oil is dissolved in water (80 mL) containing concentrated hydrochloric acid (140 mL) (any extra solvent released at this stage is separated). The aqueous solution is then refluxed overnight. After cooling to 0° C. the precipitate of benzoic acid is filtered off. (32.4 g).

### Example 8

### 6-Chloro-1-ethyl-4-morpholin-4-yl-1H-pyrazolo[3,4-d]pyrimidine (Scheme 4)

To 6-chloro-4-morpholin-4-yl-1H-pyrazolo[3,4-d]pyrimidine (100 mg, 0.418 mmol), NaH (60% in oil, 50 mg, 2.1 mmol) and ethyl iodide (168 μL, 2.1 mmol) is added N-methylpyrrolidinone (1 mL). After 5 min the reaction mixture is heated in the microwave at 175° C. for 10 min. Reverse phase HPLC gives the product as a tan powder (80 mg).

### Example 9

### 6-Chloro-4-morpholin-4-yl-1-(2-piperidin-1-yl-ethyl)-1H-pyrazolo[3,4-d]pyrimidine (Scheme 4)

To 6-chloro-4-morpholin-4-yl-1H-pyrazolo[3,4-d]pyrimidine (250 mg, 1.04 mmol), 2-piperidin-1-yl-ethanol (0.208 mL, 1.56 mmol), triphenylphosphine (409 mg, 1.56 mmol) in THF (10 mL) at 0° C. is added DIAD (0.302 mL, 1.56 mmol) by drops over 5 min. After 20 min. the reaction mixture is allowed to warm to 25° C. After 2h the mixture is concentrated in vacuo, dissolved in DMSO and purified by reverse phase HPLC giving the title compound as a TFA salt (228 mg).

### Example 10

### 4-(4-morpholin-4-yl-1-phenyl-1H-pyrazolo[3,4-d]pyrimidin-6-yl)aniline (Scheme 2)

Both 6-chloro-4-morpholin-4-yl-1-phenyl-1H-pyrazolo[3,4-d]pyrimidine (2.5 mmol, 790 mg) and 4-(4,4,5,5-tetramethyl-1,3,2-dioxaborolan-2-yl)aniline (3.0 mmol, 0.65 mg) are dissolved in a microwave vial in DME (18 mL). Na₂CO₃ (2.5 mL, 2M in water) is added along with a catalytic amount of tetrakis triphenylphosphine palladium. The mixture is heated in a sealed tube under microwave irradiation at 185° C. for 40 min. The mixture is diluted with 50 mL EtOAc and washed with a saturated solution of NaHCO₃ (2×50mL). The aqueous layers are extracted with EtOAc (50 mL) and the combined organic layers dried over MgSO₄, filtered, and concentrated in vacuo. Triturating with DCM yields 420 mg beige powder. An additional 143 mg can be obtained by triturating with EtOAc. Total yield: 563 mg (1.5 mmol).

### Example 11

### General Procedure for the Acylation of 4-(4-morpholin-4-yl-1-phenyl-1H-pyrazolo[3,4-d]pyrimidin-6-yl)aniline (Scheme 5)

To a solution of 4-(4-morpholin-4-yl-1-phenyl-1H-pyrazolo[3,4-d]pyrimidin-6-yl)aniline (40 mg, 0.1 mmol) in DMF (600 μL) is added 0.2-1 mmol of carboxylic acid and 0.2-1 mmol of IIDQ (60-300 μL). The reaction mixture is stirred for 72 hours at RT or 50° C. Several targets are bis acylated whereupon the crude acylation mixture is treated with 600 μL TFA and the mixture stirred at RT or 50° C. overnight. The crude reaction mixture contains polyesters, which are cleaved by addition of 600 μL NaOH solution (1.0 N in H₂O) followed by stirring at RT overnight. The mixture is neutralized by addition of AcOH prior to work-up. The reactions are worked up by removal of solvents under a stream of nitrogen followed by HPLC purification (Gilson, TFA or NH₄OH buffers, see table for specific conditions).

### Example 12

### N-methyl-N-[4-(4-morpholin-4-yl-1-phenyl-1H-pyrazolo[3,4-d]pyrimidin-6-yl)phenyl]acetamide (Scheme 6).

Crude methyl-[4-(4-morpholin-4-yl-1-phenyl-1H-pyrazolo[3,4-d]pyrimidin-6-yl)-phenyl]-carbamic acid tert-butyl ester (0.12 mmol assuming 100% yield), prepared according to Scheme 2, is heated, filtered and concentrated to dryness. TFA 600 μL is added and the mixture heated at 50° C. for 1.5 h. TFA is removed under a stream of nitrogen and the mixture purified by HPLC (Gilson, TFA buffers) to give N-methyl-N-[4-(4-morpholin-4-yl-1-phenyl-1H-pyrazolo[3,4-d]pyrimidin-6- yl)phenyl]acetamide.

### Example 13

### N-methyl-N-[4-(4-morpholin-4-yl-1-phenyl-1H-pyrazolo[3,4-d]pyrimidin-6-yl)phenyl]acetamide (Scheme 6)

Crude N-methyl-N-[4-(4-morpholin-4-yl-1-phenyl-1H-pyrazolo[3,4-d]pyrimidin-6-yl)phenyl]acetamide (0.12 mmol) is treated with 500 μL acetic anhydride. Upon completion of the reaction (2 h), the mixture is blown dry under a stream of nitrogen and purified by HPLC (Gilson, TFA buffers) giving the title compound.

### Example 14

### N-{4-[1-(1-benzylpiperidin-4-yl)-4-morpholin-4-yl-1H-pyrazolo[3,4-d]pyrimidin-6- yl]-2-nitrophenyl}acetamide (Scheme 5)

Crude 4-[1-(1-benzylpiperidin-4-yl)-4-morpholin-4-yl-1H-pyrazolo[3,4-d]pyrimidin-6-yl]-2-nitroaniline (0.12 mmol assuming 100% yield) is heated, filtered and rinsed with hot DME (0.5 mL). After the solvent is removed under a stream of nitrogen, 1 mL acetic anhydride, 500 μL pyridine and a catalytic amount of DMAP are added. The mixture is heated at 50° C. overnight resulting in complete di-acetylation. The solvents are removed under a stream of nitrogen. 1 mL MeOH and 1 mL of a NaOH solution (1 N in H₂O) are added. The mixture is stirred for 1 h at RT, and neutralized by addition of 1 mL AcOH. The solvents are removed under a stream of nitrogen. Purification by HPLC (Gilson, TFA buffers) gives the mono-acetylated product.

### Example 15

### 6-Chloro-4-morpholin-4-yl-1-piperidin-4-yl-1H-pyrazolo[3,4-d]pyrimidine (Scheme 7)

The HCl salt of 1-(1-benzylpiperidin-4-yl)-6-chloro-4-morpholin-4-yl-1H-pyrazolo[3,4- d]pyrimidine (100 mg, 0.24 mmol) is converted into the free base form by extraction with an aq. 1 N NaOH solution. Traces of moisture are removed by co-evaporation with 1,2-dichloroethane (DCE). The residue is dissolved in DCE (2 mL) and 1.9 mmol (0.2 mL) of alpha-chloroethyl chloroformate (ACE-Cl) is added along with a small amount of K₂CO₃ and stirred for 5.5 h at RT. The reaction is quenched by addition of MeOH and the mixture filtered and concentrated to dryness. The mixture is dissolved in MeOH and briefly heated to reflux. The title compound is obtained in quantitative yield by evaporation of the MeOH. This material can be used without further purification in the next step (reductive amination or acylation following the procedures disclosed previously, Scheme 3).

### General Procedure for Urea and Carbamate Analogs (Scheme 8)

To commercially available 3- or 4-isocyanatophenyl boronic acid, pinacol ester (49 mg, 0.2 mmol) in a microwave vial is added 1-5 mL of alcohol (enough to dissolve all isocyanatophenyl boronic acid, pinacol ester) or 1 mL of a 2 M solution of the amine in THF or 2 mL of a 0.5 M solution of the amine in dioxane. Formation of the urea or carbamate is followed by LC-MS. Upon completion of the reaction the solvents and excess amine are removed under a stream of nitrogen. The resulting urea or carbamate boronic acid pinacol ester is reacted in a Suzuki coupling without further purification.

### Example 16

### 4-[6-(1H-indol-5-yl)-4-morpholin-4-yl-1H-pyrazolo[3,4-d]pyrimidin-1-yl]cyclohexanone (Scheme 9)

1-(1,4-Dioxaspiro[4.5]dec-8-yl)-6-(1H-indol-5-yl)-4-morpholin-4-yl-1H-pyraz 3,4-d]pyrimidine (200 mg, 0.43 mmol) is treated with conc. hydrochloric acid (10 mL) and THF (20 mL) and heated at 50° C. overnight. The mixture is cooled and the precipitate collected, washed with THF giving 161 mg of the title compound (83%).

### Example 17

### General Procedure for Reductive Amination of 4-[6-(1H-indol-5-yl)-4-morpholin-4-yl-1H-pyrazolo[3,4-d]pyrimidin-1-yl]cyclohexanone (Scheme 9)

To a solution of 4-[6-(1H-indol-5-yl)-4-morpholin-4-yl-1H-pyrazolo[3,4-d]pyrimidin-1- yl]cyclohexanone (36 mg, 0.08 mmol) in THF (1.0 mL) and triethylamine (14 μL) is added the appropriate amine/aniline (0.12 mmol), zinc chloride (excess), and sodium cyanoborohydride (excess). The reaction mixture is stirred at room temperature overnight then extracted with saturated sodium bicarbonate solution and ethyl acetate. The organic layers are combined and concentrated under a stream of nitrogen to yield a crude oil. Purification by silica gel flash chromatography gives the final product.

### Example 18

### 4-[6-(1H-indol-5-yl)-4-morpholin-4-yl-1H-pyrazolo[3,4-d]pyrimidin-1-yl]cyclohexanol (Scheme 9)

To a solution of 4-[6-(1H-indol-5-yl)-4-morpholin-4-yl-1H-pyrazolo[3,4-d]pyrimidin-1-yl]cyclohexanone (36 mg, 0.08 mmol) in THF (1.0 mL) and triethylamine (14μL) is added 3-aminopyridine (11 mg, 0.12 mmol), zinc chloride (excess), and sodium cyanoborohydride (excess). The reaction mixture is stirred at room temperature overnight then extracted with saturated sodium bicarbonate solution and ethyl acetate. The organic layers are combined and concentrated under a stream of nitrogen to yield a crude oil. Purification by silica gel flash chromatography gives the final product (11 mg, 33% yield).

### Example 19

### General Method for Preparing Urea and Thiourea Compounds (Scheme 10)

To a solution of 4-[6-(1H-indol-5-yl)-4-morpholin-4-yl-1H-pyrazolo[3,4-d]pyrimidin-1- yl]cyclohexanone (50 mg, 0.12 mmol) in dichloromethane (2.0 mL) and triethylamine (excess) is added the appropriate carbamoyl chloride or isothiocyanate (0.13 mmol). The reaction mixture is stirred at room temperature for 10 min. The reaction is then extracted with water and the organics separated, dried with sodium sulfate, filtered, and concentrated in vacuo. The resulting oil is purified by silica gel chromatography to afford the urea/thiourea (8%-95%).

### Example 20

### 3-(4-morpholin-4-yl-1H-pyrazolo[3,4-d]pyrimidin-6-yl)phenol (Scheme 11)

To 6-chloro-4-morpholin-4-yl-1H-pyrazolo[3,4-d]pyrimidine (1.5 g, 6.2 mmol) in THF (25 mL) and triethylamine (excess) is added excess di-tert-butyldicarbonate (ca. 8.0 g) and refluxed overnight. The insolubles are filtered off and the filtrate extracted with water and ethyl acetate. The organic layers are combined and dried over magnesium sulfate and then filtered through a pad of hydrous magnesium silicate and concentrated in vacuo. The crude solid is purified by silica gel chromatography affording 0.6 g of tert-butyl 6-chloro-4-morpholin-4-yl-1H-pyrazolo[3,4-d]pyrimidine-1-carboxylate (28%). This compound (50 mg, 0.15 mmol) is treated under the Suzuki conditions (Scheme 2) to give the title compound (4 mg, 9% yield).

### Example 21

### Preparation of 6-(1H-indol-5-yl)-4-morpholin-4-yl-1-(1-pyridin-2-ylpiperidin-4-yl)-1H-pyrazolo[3,4-d]pyrimidine (Scheme 12)

6-(1H-indol-5-yl)-4-morpholin-4-yl-1-piperidin-4-yl-1H-pyrazolo[3,4-d]pyrimidine (60 mg, 0.15 mmol) is dissolved in excess 2-bromopyridine (ca. 0.5 mL) and heated to 100° C. for 3 days. The mixture is cooled and partitioned between water and ethyl acetate. The organic layer is dried over magnesium sulfate, filtered and concentrated in vacuo to give the title compound as a solid (30 mg, 42%).

### Example 22

### Preparation of N-(4-(4-morpholino-1H-pyrazolo[3,4-d]pyrimidin-6-yl)phenyl)acetamide (Scheme 13)

6-Chloro-4-morpholin-4-yl-1H-pyrazolo[3,4-d]pyrimidine (5.0 g, 21 mmol) is taken up as a suspension in dry ethyl acetate (50 mL). Following the addition of 4-toluenesulfonic acid monohydrate (25 mg), the mixture is heated to 60° C. and 3,4-dihydro-2H-pyran (2.5 mL) is added by drops. The reaction mixture is maintained at 60° C. for 18 hours and then concentrated under reduced pressure. The residue is purified by flash silica gel chromatography. Following concentration of fractions, 4-(6-chloro-1-(tetrahydro-2H-pyran-2-yl)-1H-pyrazolo[3,4-d]pyrimidin-4-yl)morpholine is obtained as a granular yellow solid (3.2 g, 47%). 4-(6-chloro-1-(tetrahydro-2H-pyran-2-yl)-1H-pyrazolo[3,4-d]pyrimidin-4-yl)morpholine (1.0 g, 3.1 mmol) is coupled to 4-acetamidophenylboronic acid (0.83 g, 4.6 mmol) with tetrakis(triphenylphosphine) palladium (0) (2.5 mol %), 2 M aqueous sodium carbonate solution, and ethylene glycol dimethyl ether (DME), under microwave irradiation (175° C., 10 min). Following an aqueous workup and flash chromatography, N-(4-(4-morpholino-1-(tetrahydro-2H-pyran-2-yl)-1H-pyrazolo[3,4-d]pyrimidin-6-yl)phenyl)acetamide is obtained as a light tan foam, which could be crushed to a powder (0.93 g, 72%). N-(4-(4-morpholino-1-(tetrahydro-2H-pyran-2-yl)-1H-pyrazolo[3,4-d]pyrimidin-6-yl)phenyl)acetamide (0.50 g, 1.2 mmol) is taken up in dioxane (5 mL), treated with 4 M hydrogen chloride in dioxane (5 mL), and allowed to stir at room temperature for 3 days. The slurry is then concentrated and purified by reverse phase preparative high performance liquid chromatography, employing a Phenomenex Prodigy 250 mm×21.2 mm 5 μm column and a 5% acetonitrile/95% water/0.1% trifluoroacetic acid to 100% acetonitrile gradient over 40 minutes. After concentration, N-(4-(4-morpholino-1H-pyrazolo[3,4-d]pyrimidin-6-yl)phenyl)acetamide (4) is provided as a solid.

### Example 23

### Preparation of ethyl 4-(4-morpholino-6-(4-(3-phenylureido)phenyl)-1H-pyrazolo[3,4-d]pyrimidin-1-yl)piperidine-1-carboxylate (Scheme 22)

tert-Butyl 4-(6-chloro-4-morpholino-1H-pyrazolo[3,4-d]pyrimidin-1-yl)piperidine-1-carboxylate is coupled to 4-nitrophenylboronic acid pinacol ester (1.5 eq) with tetrakis(triphenylphosphine) palladium (0) (10 mol %), 2 M aqueous sodium carbonate solution (4 eq), and ethylene glycol dimethyl ether (DME), under microwave irradiation (150° C., 10 min). Following filtration over celite and subjection of the filtrate to aqueous workup, flash chromatography provides tert-butyl 4-(4-morpholino-6-(4-nitrophenyl)-1H-pyrazolo[3,4-d]pyrimidin-1-yl)piperidine-1-carboxylate.

tert-Butyl 4-(4-morpholino-6-(4-nitrophenyl)-1H-pyrazolo[3,4-d]pyrimidin-1-yl)piperidine-1-carboxylate is treated with a mixture of TFA in CH₂Cl₂ (25% TFA in CH₂Cl₂, v/v) and the solution is stirred at room temperature for 3 hours. The solution was concentrated and treated with CH₂Cl₂ and 0.2N NaOH. The organic phase is dried and concentrated, then treated with Et₃N (2.5 eq) and RCOCl (1.2 eq) in CH₂Cl₂ and stirred at room temperature for 15 hours. Aqueous workup and concentration provides ethyl 4-(4-morpholino-6-(4-nitrophenyl)-1H-pyrazolo[3,4-d]pyrimidin-1-yl)piperidine-1-carboxylate.

Ethyl 4-(4-morpholino-6-(4-nitrophenyl)-1H-pyrazolo[3,4-d]pyrimidin-1-yl)piperidine-1-carboxylate is dissolved in EtOH and CH₂Cl₂ (1:1, v/v) and palladium on carbon (approx. 1 g for 10 mmol of substrate) is added. The reaction vessel is purged with H₂ gas and stirred under H₂ atmosphere for 22 hours. The suspension is filtered over celite and the filtrate is concentrated to provide ethyl 4-(6-(4-aminophenyl)-4-morpholino-1H-pyrazolo[3,4-d]pyrimidin-1-yl)piperidine-1-carboxylate.

A solution of triphosgene (0.5 eq) in CH₂Cl₂ is prepared. To this is added a solution of ethyl 4-(6-(4-aminophenyl)-4-morpholino-1H-pyrazolo[3,4-d]pyrimidin-1-yl)piperidine-1-carboxylate in CH₂Cl₂ and Et₃N (3 eq) and the reaction is stirred at room temperature for 15 minutes. The resulting solution is then transferred to a vessel containing aniline (5 eq) in CH₂Cl₂ and the mixtures are stirred at room temperature for 16 hours. The solutions are concentrated and purified by reverse phase preparative high performance liquid chromatography to provide ethyl 4-(4-morpholino-6-(4-(3-phenylureido)phenyl)-1H-pyrazolo[3,4-d]pyrimidin-1-yl)piperidine-1-carboxylate.

### Example 24

### Preparation of methyl 4-(6-(4-(3-(4-fluorophenyl)ureido)phenyl)-4-morpholino-1H-pyrazolo[3,4-d]pyrimidin-1-yl)piperidine-1-carboxylate (Scheme 23)

Methyl 4-(6-chloro-4-morpholino-1H-pyrazolo[3,4-d]pyrimidin-1-yl)piperidine-1-carboxylate is coupled to 4-aminophenylboronic acid pinacol ester (1.3 eq) with tetrakis(triphenylphosphine) palladium (0) (10 mol %), 2 M aqueous sodium carbonate solution (4 eq), and toluene/ethanol (1:1, v/v) by heating to 85° C. via oil bath for 16 hours or to 120° C. by microwave irradiation for 30 minutes. Following filtration over celite and subjection of the filtrate to aqueous workup, flash chromatography provides methyl 4-(6-(4-aminophenyl)-4-morpholino-1H-pyrazolo[3,4-d]pyrimidin-1-yl)piperidine-1-carboxylate.

A solution of triphosgene (0.5 eq) in CH₂Cl₂ is prepared. To this is added a solution of methyl 4-(6-(4-aminophenyl)-4-morpholino-1H-pyrazolo[3,4-d]pyrimidin-1-yl)piperidine-1-carboxylate in CH₂Cl₂ and Et₃N (3 eq) and the reaction is stirred at room temperature for 15 minutes. The resulting solution is then transferred to a vessel containing 4-fluoroaniline (3 eq) in CH₂Cl₂ and the mixtures are stirred at room temperature for 16 hours. The solutions are concentrated and purified by reverse phase preparative high performance liquid chromatography to provide methyl 4-(6-(4-(3-(4-fluorophenyl)ureido)phenyl)-4-morpholino-1H-pyrazolo[3,4-d]pyrimidin-1-yl)piperidine-1-carboxylate.

### Example 25

### Preparation of ethyl 4-(6-(4-(1H-imidazol-2-ylamino)phenyl)-4-morpholino-1H-pyrazolo[3,4-d]pyrimidin-1-yl)piperidine-1-carboxylate (Scheme 24)

Ethyl 4-(6-(4-aminophenyl)-4-morpholino-1H-pyrazolo[3,4-d]pyrimidin-1-yl)piperidine-1-carboxylate is dissolved in ethanol and 2,2-diethoxy-N-(iminomethylene)ethanamine (1.1 eq, prepared via the method described in J. Med. Chem., 1997, 40, 18-23) and methanesulfonic acid (1 eq) are added. The solution is heated to reflux for 15 hours. Additional 2,2-diethoxy-N-(iminomethylene)ethanamine (13 eq) and methanesulfonic acid (13 eq) were added and the reaction is heated to reflux for an additional 32 hours. The solution is poured into 1M NaOH, extracted with CH₂Cl₂, dried, and concentrated. Purification by reverse phase preparative high performance liquid chromatography provides ethyl 4-(6-(4-(1H-imidazol-2-ylamino)phenyl)-4-morpholino-1H-pyrazolo[3,4-d]pyrimidin-1-yl)piperidine-1-carboxylate.

### Example 26

### Preparation of methyl 4-(4-morpholino-6-(4-(3-(4-(2-(pyrrolidin-1-yl)ethyl)phenyl)ureido)phenyl)-1H-pyrazolo[3,4-d]pyrimidin-1-yl)piperidine-1-carboxylate (Scheme 25)

Methyl 4-(6-(4-(3-(4-(2-hydroxyethyl)phenyl)ureido)phenyl)-4-morpholino-1H-pyrazolo[3,4-d]pyrimidin-1-yl)piperidine-1-carboxylate is dissolved in CH₂Cl₂ and Et₃N (5 eq) and TsCl (1.5 eq) are added. The solution is washed with sat. NaHCO₃, brine, dried, and concentrated. The crude product is dissolved in CH₂Cl₂ and pyrrolidine (10 eq) is added. The solution is stirred at RT for 4 hours, concentrated, and purified by reverse phase preparative high performance liquid chromatography to provide methyl 4-(4-morpholino-6-(4-(3-(4-(2-(pyrrolidin-1-yl)ethyl)phenyl)ureido)phenyl)-1H-pyrazolo[3,4-d]pyrimidin-1-yl)piperidine-1-carboxylate.

### Example 27

### Preparation of methyl 4-(6-(4-hydroxyphenyl)-4-morpholino-1H-pyrazolo[3,4-d]pyrimidin-1-yl)piperidine-1-carboxylate and methyl 4-(6-(4-(methylcarbamoyloxy)phenyl)-4-morpholino-1H-pyrazolo[3,4-d]pyrimidin-1-yl)piperidine-1-carboxylate (Scheme 26)

Methyl 4-(6-chloro-4-morpholino-1H-pyrazolo[3,4-d]pyrimidin-1-yl)piperidine-1-carboxylate is coupled to 4-hydroxyphenylboronic acid pinacol ester (1.3 eq) with tetrakis(triphenylphosphine) palladium (0) (10 mol %), 2 M aqueous sodium carbonate solution (4 eq), and DME by heating to 150° C. by microwave irradiation for 30 minutes. Following filtration over celite and subjection of the filtrate to aqueous workup, flash chromatography provides methyl 4-(6-(4-hydroxyphenyl)-4-morpholino-1H-pyrazolo[3,4-d]pyrimidin-1-yl)piperidine-1-carboxylate.

A solution of triphosgene (0.5 eq) in CH₂Cl₂ is prepared. To this is added a solution of methyl 4-(6-(4-hydroxyphenyl)-4-morpholino-1H-pyrazolo[3,4-d]pyrimidin-1-yl)piperidine-1-carboxylate in CH₂Cl₂ and Et₃N (3 eq) and the reaction is stirred at room temperature for 15 minutes. The resulting solution is then transferred to a vessel containing methylamine in 2.0 M THF (5 eq) in CH₂Cl₂ and the mixtures are stirred at room temperature for 16 hours. The solutions are concentrated and purified by reverse phase preparative high performance liquid chromatography to provide methyl 4-(6-(4-(methylcarbamoyloxy)phenyl)-4-morpholino-1H-pyrazolo[3,4-d]pyrimidin-1-yl)piperidine-1-carboxylate.

### Example 28

### Preparation of methyl 4-(4-morpholino-6-(4-(phenoxycarbonylamino)phenyl)-1H-pyrazolo[3,4-d]pyrimidin-1-yl)piperidine-1-carboxylate (Scheme 27)

Methyl 4-(6-(4-aminophenyl)-4-morpholino-1H-pyrazolo[3,4-d]pyrimidin-1-yl)piperidine-1-carboxylate is treated with Et₃N (1.1 eq) and phenyl chloroformate (1.2 eq) and stirred at RT for 4 hours. Aqueous workup and purification by flash chromatography provides methyl 4-(4-morpholino-6-(4-(phenoxycarbonylamino)phenyl)-1H-pyrazolo[3,4-d]pyrimidin-1-yl)piperidine-1-carboxylate.

### Example 29

### Preparation of tert-butyl 4-(6-(4-(N-methylsulfamoylamino)phenyl)-4-morpholino-1H-pyrazolo[3,4-d]pyrimidin-1-yl)piperidine-1-carboxylate (Scheme 28)

tert-Butyl 4-(6-(4-aminophenyl)-4-morpholino-1H-pyrazolo[3,4-d]pyrimidin-1-yl)piperidine-1-carboxylate is treated with methyl sulfamoyl chloride ((prepared using the procedure described in: J. Med. Chem., 1983, 26, 1077-1079), 2 eq) and pyridine (4 eq) in DMF and allowed to stir at RT for 3.5 hours. Concentration and purification by reverse phase preparative high performance liquid chromatography provide tert-butyl 4-(6-(4-(N-methylsulfamoylamino)phenyl)-4-morpholino-1H-pyrazolo[3,4-d]pyrimidin-1-yl)piperidine-1-carboxylate.

### Scheme 29: General Procedure

49 mg (0.2 mmol) 3-isocyanatophenylboronic acid, pinacol ester is dissolved in a solution of the appropriate nucleophile (5 mL MeOH; 1 mL of a 2N solution of MeNH2 in THF; 2 mL of a 0.5 N solution of NH3 in dioxane) and stirred for 30 min at room temperature. The solvents are evaporated and 50 mg (0.12 mmol) 1-(1-Benzyl-piperidin-4-yl)-6-chloro-4-morpholin-4-yl-1H-pyrazolo[3,4-d]pyrimidine is added. The mixture is dissolved in 2 mL DME and 250 uL of a 2M Na2CO3 solution is added, followed by addition of Pd(PP3)4 (10 mol %). The mixture is heated under microwave irradiation for 6 min at 185 C. The solvents are evaporated and the mixture is purified by HPLC (TFA buffers).

### Example 30

### 1-{3-[1-(1-Benzyl-piperidin-4-yl)-4-morpholin-4-yl-1H-pyrazolo[3,4-d]pyrimidin-6-yl]-phenyl}-3-methyl-urea (Scheme 29)

49 mg (0.2 mmol) 3-isocyanatophenylboronic acid, pinacol ester is dissolved in 1 mL of a 2N solution of MeNH2 in THF and stirred for 30 min at room temperature. The solvents are evaporated and 50 mg (0.12 mmol) 1-(1-Benzyl-piperidin-4-yl)-6-chloro-4-morpholin-4-yl-1H-pyrazolo[3,4-d]pyrimidine is added. The mixture is dissolved in 2 mL DME and 250 uL of a 2M Na2CO3 solution is added, followed by addition of Pd(PP3)4 (10 mol %). The mixture is heated under microwave irradiation for 6 min at 185 C. The solvents are evaporated and the mixture is purified by HPLC (TFA buffers) to give 1-{3-[1-(1-Benzyl-piperidin-4-yl)-4-morpholin-4-yl-1H-pyrazolo[3,4-d]pyrimidin-6-yl]-phenyl}-3-methyl-urea.

### Example 31

### N-[4-(4-morpholin-4-yl-1-phenyl-1H-pyrazolo[3,4-d]pyrimidin-6-yl)phenyl]hydrazinecarboxamide (Scheme 30)

37 mg (0.1 mmol) 4-(4-Morpholin-4-yl-1-phenyl-1H-pyrazolo[3,4-d]pyrimidin-6-yl)-phenylamine is suspended in 2 mL DCM containing 65 uL Net3. 250 uL 20% phosgene in toluene is added to give a clear solution. After 30 min at RT, 2 mmol (63 uL) hydrazine is added. The reaction is allowed to proceed overnight. The solvents are evaporated and the mixture is purified by HPLC (TFA buffers) to give the title compound.

### Example 32

### N-{4-[1-(1-Benzyl-piperidin-4-yl)-4-morpholin-4-yl-1H-pyrazolo[3,4-d]pyrimidin-6-yl]-phenyl}-2,2,2-trifluoro-acetamide (Scheme 31)

118 mg of the TFA salt of 4-[1-(1-Benzyl-piperidin-4-yl)-4-morpholin-4-yl-1H-pyrazolo[3,4-d]pyrimidin-6-yl]-phenylamine was dissolved in DCM (5 mL). 165 uL Net3 was added, followed by addition of 50 mg triphosgene. After 30 min stirring at room temperature, the solvents were evaporated and the mixture was purified by HPLC (TFA buffers) to give 53 mg of the title compound.

### Example 33

### 1-{4-[1-(1-Benzyl-piperidin-4-yl)-4-morpholin-4-yl-1H-pyrazolo[3,4-d]pyrimidin-6-yl]-phenyl}-3-(2-methylamino-ethyl)-urea (Scheme 32)

49 mg (0.2 mmol) 4-isocyanatophenylboronic acid, pinacol ester was dissolved in 2 mL DME. 0.2 mmol (36 uL) N-Me-N-Boc ethylene diamine was added. The mixture was stirred for 30 min at room temperature. 50 mg 1-(1-Benzyl-piperidin-4-yl)-6-chloro-4-morpholin-4-yl-1H-pyrazolo[3,4-d]pyrimidine was added, followed by 250 uL of a 2M solution of Na2CO3 and 10 mol % Pd(PPh3)4. The mixture was heated under microwave irradiation at 185 C for 6 min. The mixture was cooled to room temperature and 2 mL TFA was added. After stirring at room temperature for 3h the solvents were removed and the mixture was purified by HPLC (TFA buffers) to give 34 mg of the title compound.

### Example 34

### 1-{4-[1-(1-Benzyl-piperidin-4-yl)-4-morpholin-4-yl-1H-pyrazolo[3,4-d]pyrimidin-6-yl]-phenyl}-3-methyl-thiourea (Scheme 15)

0.11 mmol of 4-[1-(1-Benzyl-piperidin-4-yl)-4-morpholin-4-yl-1H-pyrazolo[3,4-d]pyrimidin-6-yl]-phenylamine was dissolved in DCM (1 mL). 65 uL NEt3 was added, followed by addition of 75 uL methylisothiocyanate. The mixture was stirred at 50 C overnight, the solvents were evaporated and the mixture was purified by HPLC (TFA buffers).

### Example 35

### 5-[1-(1-Benzyl-piperidin-4-yl)-4-morpholin-4-yl-1H-pyrazolo[3,4-d]pyrimidin-6-yl]-1,3-dihydro-benzoimidazol-2-one (Scheme 33)

53 mg (0.2 mmol) 4-amino-3-nitrophenylboronic acid, pinacol ester was suspended in 2 mL DME. A catalytic amount of Pd/C was added and the nitro group was reduced under an atmosphere of hydrogen over 4 days. 130 uL Net3 was added followed by 30 mg triphosgene. The mixture was stirred for 15 min and 50 mg 1-(1-Benzyl-piperidin-4-yl)-6-chloro-4-morpholin-4-yl-1H-pyrazolo[3,4-d]pyrimidine was added, followed by 250 uL of a 2M solution of Na2CO3 and 10 mol % Pd(PPh3)4. The mixture was heated under microwave irradiation at 185 C for 6 min. The solvents were evaporated and the mixture was purified by HPLC (TFA buffers).

### Examples 36, 37, 38

### 4-[1-(1-Benzyl-piperidin-4-yl)-4-morpholin-4-yl-1H-pyrazolo[3,4-d]pyrimidin-6-yl]phenylamine

### {4-[1-(1-Benzyl-piperidin-4-yl)-4-morpholin-4-yl-1H-pyrazolo[3,4-d]pyrimidin-6-yl]-2-fluoro-phenyl}-3-methyl-urea

### 1-{4-[1-(1-Benzyl-piperidin-4-yl)-4-morpholin-4-yl-1H-pyrazolo[3,4-d]pyrimidin-6-yl]-2-fluoro-phenyl}-3-ethyl-urea (Scheme 34)

150 mg (0.33 mmol) 1-(1-Benzyl-piperidin-4-yl)-6-chloro-4-morpholin-4-yl-1H-pyrazolo[3,4-d]pyrimidine was dissolved in 6 mL DME. 750 uL of a 2M solution of Na2CO3 was added followed by the addition of 117 mg (0.46 mmol) 4-N-Boc-amino-3-fluoro-phenylboronic acid and 30 mg Pd(PPh3)4. The mixture was heated under microwave irradiation at 185 C for 30 min. The mixture was cooled to room temperature, diluted with ethyl acetate and filtered over Celite. The organic phase was washed with a sat. solution of NaHCO3, dried over MgSO4 and concentrated. The crude Suzuki product was dissolved in 2 mL DCM and 2 mL TFA was added. After 30 min at room temperature, the solvents were removed under reduced pressure. The deprotected aniline was dissolved in DCM and washed with a sat. solution of NaHCO3. The organic phase was dried over MgSO4, concentrated and dissolved in DCM and split over 3 vials. 1 vial was purified by HPLC (TFA buffers) to give the free aniline 4-[1-(1-Benzyl-piperidin-4-yl)-4-morpholin-4-yl-1H-pyrazolo[3,4-d]pyrimidin-6-yl]-2-fluoro-phenylamine. To each of the remaining two vials was added 15 mg triphosgene and 65 uL Net3. After 5 min at room temperature, 1 mL of a 2N solution of MeNH2 or EtNH2 in THF was added. After 30 min, the solvents were evaporated and the mixtures were purified by HPLC (TFA buffers) to give the urea products {4-[1-(1-Benzyl-piperidin-4-yl)-4-morpholin-4-yl-1H-pyrazolo[3,4-d]pyrimidin-6-yl]-2-fluoro-phenyl}-3-methyl-urea and 1-{4-[1-(1-Benzyl-piperidin-4-yl)-4-morpholin-4-yl-1H-pyrazolo[3,4-d]pyrimidin-6-yl]-2-fluoro-phenyl}-3-ethyl-urea.

### Examples 39, 40, 41, 42, 43, 44

### 1-2-Fluoro-4-[4-morpholin-4-yl-1-(1-pyridin-3-ylmethyl-piperidin-4-yl)-1H-pyrazolo[3,4-d]pyrimidin-6-yl]-phenyl}-3-methyl-urea

### 1-2-Fluoro-4-[4-morpholin-4-yl-1-(1-pyridin-3-ylmethyl-piperidin-4-yl)-1H-pyrazolo[3,4-d]pyrimidin-6-yl]-phenyl}-3-ethyl-urea

### 1-2-Fluoro-4-[4-morpholin-4-yl-1-(1-pyridin-3-ylmethyl-piperidin-4-yl)-1H-pyrazolo[3,4-d]pyrimidin-6-yl]-phenyl}-3-phenyl-urea

### 1-2-Fluoro-4-[4-morpholin-4-yl-1-(1-pyridin-3-ylmethyl-piperidin-4-yl)-1H-pyrazolo[3,4-d]pyrimidin-6-yl]-phenyl}-3-pyridin-3-yl-urea

### 1-2-Fluoro-4-[4-morpholin-4-yl-1-(1-pyridin-3-ylmethyl-piperidin-4-yl)-1H-pyrazolo[3,4-d]pyrimidin-6-yl]-phenyl}-3-pyridin-4-yl-urea

### 1-(2-Fluoro-ethyl)-3-{2-fluoro-4-[4-morpholin-4-yl-1-(1-pyridin-3-ylmethyl-piperidin-4-yl)-1H-pyrazolo[3,4-d]pyrimidin-6-yl]-phenyl}-urea (Scheme 35)

1.18 mmol 6-Chloro-4-morpholin-4-yl-1-(1-pyridin-3-ylmethyl-piperidin-4-yl)-1H-pyrazolo[3,4-d]pyrimidine was dissolved in 17.5 mL DME. 2.5 mL of a 2M solution of Na2CO3 was added followed by the addition of 392 mg (1.5 mmol) 4-N-Boc-amino-3-fluoro-phenylboronic acid and 100 mg Pd(PPh3)4. The mixture was heated under microwave irradiation at 185 C for 30 min. An additional 100 mg Pd(PPh3)4 was added and the mixture was heated again under microwave irradiation at 185 C for 30 min. LCMS revealed that the reaction was complete and that the Boc group had been removed. The mixture was cooled to room temperature, diluted with ethyl acetate and filtered over Celite. The organic phase was washed with a sat. solution of NaHCO3, dried over MgSO4 and concentrated.

The crude aniline was dissolved in 12 mL DCM and 650 uL Net3 was added. 150 mg triphosgene was added and the mixture was stirred at room temperature for 10 min. 2 mL of this solution is added to each of 6 vials containing 1-2 mmol of amine as follows:
1. 1 mL of a 2N solution of MeNH2 in THF
2. 1 mL of a 2N solution of EtNH2 in THF
3. 1 mmol (93 uL) aniline in 1 mL DCM
4. 1 mmol (94 mg) 3-aminopyridine in 1 mL DCM
5. 1 mmol (94 mg) 4-aminopyridine in 1 mL DCM
6. 1 mmol (99 mg) 2-fluoroethylamine.HCl in 1 mL IN NaOH.

After 30 min, the solvents were evaporated and the mixtures were purified by HPLC (TFA buffers) to give the urea products.

### Example 45

### 1-{4-[4-Morpholin-4-yl-1-(1-pyridin-3-ylmethyl-piperidin-4-yl)-1H-pyrazolo[3,4-d]pyrimidin-6-yl]-phenyl}-3-thiophen-3-yl-urea (Scheme 14)

50 mg 4-[4-Morpholin-4-yl-1-(1-pyridin-3-ylmethyl-piperidin-4-yl)-1H-pyrazolo[3,4-d]pyrimidin-6-yl]-phenylamine was dissolved in 1 mL DCM. 65 uL Net3 was added followed by addition of 0.2 mmol 3-thienylisocyanate. The mixture was stirred at room temperature overnight. The solvents were evaporated and the product was purified by HPLC (TFA buffers).

### Example 46

### 6-(2,3-Dihydro-1H-indol-5-yl)-4-morpholin-4-yl-1-(1-pyridin-3-ylmethyl-piperidin-4-yl)-1H-pyrazolo[3,4-d]pyrimidine (Scheme 36)

1 mmol (198 mg) 5-bromoindoline, 1.5 mmol (381 mg) bispinacolatodiboron, 0.1 mmol (73 mg) Pd(dppf)Cl2.CH2Cl2 and 3 mmol (296 mg) KOAc are suspended in DMSO and heated at 80 C overnight. The mixture is diluted with EtOAc, filtered over Celite and concentrated. Flash chromatography (5-20% EtOAc in hexanes) gave 69 mg (28%) of the boronate. This was added to a solution of 50 mg 6-Chloro-4-morpholin-4-yl-1-(1-pyridin-3-ylmethyl-piperidin-4-yl)-1H-pyrazolo[3,4-d]pyrimidine in 2 mL DME and 250 uL of a 2M Na2CO3 solution. 10 mol % Pd(PP3)4 was added and the mixture was stirred at 95C overnight. The solvents were removed and the product was purified by HPLC (TFA buffers).

### Example 47

### 1-Methyl-3-(5-{4-morpholin-4-yl-1-[1-(pyridine-3-carbonyl)-piperidin-4-yl]-1H-pyrazolo[3,4-d]pyrimidin-6-yl}-pyridin-2-yl)-urea (Scheme 37)

44 mg 2-aminopyridine-5-boronic acid, pinacol ester is dissolved in 2 mL DCM. 130 uL NET3 is added followed by 30 mg triphosgene. After 10 min, 1 mL of a 2N solution of MeNH2 in THF is added. After 30 min, the solvents are evaporated. 50 mg of [4-(6-chloro-4-morpholin-4-yl-pyrazolo[3,4-d]pyrimidin-1-yl)-piperidin-1-yl]-pyridin-3-yl-methanone is added and the mixture is dissolved in 2 mL DME. 250 uL of a 2M solution of Na2CO3 is added followed by the addition of 10 mol % Pd(PPh3)4. The mixture is heated under microwave irradiation at 185 C for 10 min. The solvents are evaporated and the product is purified by HPLC (TFA buffers).

### Example 48

### 1-{2-Chloro-4-[4-morpholin-4-yl-1-(1-pyridin-3-ylmethyl-piperidin-4-yl)-1H-pyrazolo[3,4-d]pyrimidin-6-yl]-phenyl}-3-methyl-urea (Scheme 38)

12 mg (0.02 mmol) 1-Methyl-3-{4-[4-morpholin-4-yl-1-(1-pyridin-3-ylmethyl-piperidin-4-yl)-1H-pyrazolo[3,4-d]pyrimidin-6-yl]-phenyl}-urea and 4 mg (0.03 mmol) NCS are dissolved in DCM and heated under reflux over the weekend. HPLC purification (TFA buffers) gave 4.6 mg (0.007 mmol, 32%) of the product.

### Example 49

### 4-(6-Chloro-4-morpholin-4-yl-pyrazolo[3,4-d]pyrimidin-1-yl)-piperidine-1-carboxylic acid methyl ester (Scheme 39)

499 mg (1.2 mmol) 1-(1-Benzyl-piperidin-4-yl)-6-chloro-4-morpholin-4-yl-1H-pyrazolo[3,4-d]pyrimidine is dissolved in 0.4 mL (5.2 mmol) methylchloroformate and 1 mL DCM. After 3 h at RT the solvents are removed under reduced pressure.

### Examples 50-55

### 1-(4-{1-[1-(4-Hydroxy-butyl)-piperidin-4-yl]-4-morpholin-4-yl-1H-pyrazolo[3,4-d]pyrimidin-6-yl}-phenyl)-3-methyl-urea

### (4-{1-[1-(4-Hydroxy-butyl)-piperidin-4-yl]-4-morpholin-4-yl-1H-pyrazolo[3,4-d]pyrimidin-6-yl}-phenyl)-carbamic acid methyl ester

### 1-Ethyl-3-(4-{1-[1-(4-hydroxy-butyl)-piperidin-4-yl]-4-morpholin-4-yl-1H-pyrazolo[3,4-d]pyrimidin-6-yl}-phenyl)-urea

### 1-(4-{1-[1-(4-Hydroxy-butyl)-piperidin-4-yl]-4-morpholin-4-yl-1H-pyrazolo[3,4-d]pyrimidin-6-yl}-phenyl)-3-(2-hydroxy-ethyl)-urea

### 1-(2-Fluoro-ethyl)-3-(4-{1-[1-(4-hydroxy-butyl)-piperidin-4-yl]-4-morpholin-4-yl-1H-pyrazolo[3,4-d]pyrimidin-6-yl}-phenyl)-urea

### 1-(4-{1-[1-(4-Hydroxy-butyl)-piperidin-4-yl]-4-morpholin-4-yl-1H-pyrazolo[3,4-d]pyrimidin-6-yl}-phenyl)-3-phenyl-urea (Scheme 40)

8.3g (20.9 mmol) 6-Chloro-4-morpholin-4-yl-1-piperidin-4-yl-1H-pyrazolo[3,4-d]pyrimidine.2HCl was suspended in 170 mL THF. 29 mmol (2.8 mL) 3-pyridinecarboaxaldehyde, 1.25 mL acetic acid and 29 mmol (6.9 g) NaHB(OAc)3 were added and the mixture was stirred for 48 h. The mixture was diluted with ethyl acetate and washed with a sat. solution of NaHCO3. The organic phase was dried over MgSO4 and concentrated and purified by silica gel chromatography (0-20% MeOH in EtOAc containing 1% Net3) to give the hydroxybutylated product as a white solid (1.33g, 3.4 mmol, 27%).

49 mg (0.2 mmol) 4-isocyanatophenylboronic acid, pinacol ester was dissolved in 1 mL DME. To this solution was added amine or alcohol as follows:
1) 1 mL of a 2N solution of MeNH2 in THF
2) 2 mL MeOH
3) 1 mL of a 2N solution of EtNH2 in THF
4) 0.2 mmol ethanolamine in 200 uL DME
5) 0.2 mmol 2-fluoroethylamine.HCl in 200 uL IN NaOH
6) 0.2 mmol aniline in 200 uL DME

In case of 1), 2) and 3), the solvents were evaporated after 2.5 h and the sample was redissolved in 1 mL DME

In case of 5), the sample was diluted with EtOAc after 2.5 h and washed with water. The solvents were evaporated and the sample was redissolved in 1 mL DME.

In case of 4) and 6), no work-up was done.

To the thus obtained DME solutions of ureidophenylboronate and carbamoylphenylboronate was added 50 mg of 4-[4-(6-Chloro-4-morpholin-4-yl-pyrazolo[3,4-d]pyrimidin-1-yl)-piperidin-1-yl]-butan-1-ol, 250 uL of a 2M solution of Na2CO3 and 10 mol % Pd(PPh3)4. The sample was heated under microwave irradiation for 6 min at 185C and the product was purified by HPLC (TFA buffers).

### Example 56

### 1-Methyl-3-[4-(4-morpholin-4-yl-1-piperidin-4-yl-1H-pyrazolo[3,4-d]pyrimidin-6-yl)-phenyl]-urea (Scheme 41)

To a solution of 0.1 mmol 4-{6-[4-(3-Methyl-ureido)-phenyl]-4-morpholin-4-yl-pyrazolo[3,4-d]pyrimidin-1-yl}-piperidine-1-carboxylic acid tert-butyl ester in 2.5 mL DCM was added 2.5 mL TFA. After 2 h the solvents were removed and the product was purified by HPLC (TFA buffers).

### Example 57

### 4-{6-[4-(2,3-Dimethyl-isothioureido)-phenyl]-4-morpholin-4-yl-pyrazolo[3,4-d]pyrimidin-1-yl}-piperidine-1-carboxylic acid isopropyl ester (Scheme 42)

23.5 mg (0.04 mmol) 4-{6-[4-(3-Methyl-thioureido)-phenyl]-4-morpholin-4-yl-pyrazolo[3,4-d]pyrimidin-1-yl}-piperidine-1-carboxylic acid isopropyl ester is dissolved in acetone. Excess K2CO3 is added, followed by addition of 1 mmol (62 uL) MeI. The reaction is allowed to proceed for 5 h. The mixture is filtered, concentrated, dissolved in DCM and washed with water. The organic phase is dried over MgSO4 and concentrated.

### Example 58

### 3-methoxy-N-[4-(4-morpholin-4-yl-1H-pyrazolo[3,4-d]pyrimidin-6-yl)phenyl]benzamide (Scheme 43)

4-[4-Morpholin-4-yl-1-(tetrahydro-2H-pyran-2-yl)-1H-pyrazolo[3,4-d]pyrimidin-6-yl]aniline (100 mg) and excess triethylamine in tetrahydrofuran (2.0 mL) were treated with the acid chloride (excess). After 18 h the volatiles were removed in vacuo and the residue partitioned between water and dichloromethane. The organic phase was dried with magnesium sulfate and concentrated to an oil that was purified by silica gel chromatography to give the title compound (43 mg).

### Example 59

### 1-[2-chloro-4-(4-morpholin-4-yl-1H-pyrazolo[3,4-d]pyrimidin-6-yl)phenyl]-3-methylurea (Scheme 44)

1-Methyl-3-{4-[4-morpholin-4-yl-1-(tetrahydro-2H-pyran-2-yl)-1H-pyrazolo[3,4-d]pyrimidin-6-yl]phenyl} (500 mg) and N-chlorosuccinimide (excess) in tetrahydrofuran (10.0 mL) was heated at 40 C for 1 h. The reaction was partitioned between water and ethyl acetate. The organic phase was dried with magnesium sulfate and evaporated to give the title compound as a solid (200 mg).

### Example 60

### 3-(6-Chloro-1-ethyl-4-morpholin-4-yl-1H-pyrazolo[3,4-d]pyrimidin-3-yl)prop-2-yn-1-ol (Scheme 45)

6-Chloro-4-morpholin-4-yl-1H-pyrazolo[3,4-d]pyrimidine (2.0 g) and N-Todosuccinimide (excess) in dimethylformamide (10.0 mL) were heated at 80 C for 48 h. The reaction was partitioned between water and ethyl acetate. The organic phase was separated and dried with magnesium sulfate and evaporated leaving a crude solid that was purified by silica gel column to give 6-chloro-3-iodo-4-morpholin-4-yl-1H-pyrazolo[3,4-d]pyrimidine (680 mg).

6-Chloro-3-iodo-4-morpholin-4-yl-1H-pyrazolo[3,4-d]pyrimidine (1.9 g) was dissolved in dimethylformamide (10 mL) and potassium carbonate (1.0 g) and ethyl iodide (1.5 eq.) were added. After 18 h the reaction mixture was poured into water (500 mL). The resulting precipitate was filtered and washed with water to give 6-chloro-1-ethyl-3-iodo-4-morpholin-4-yl-1H-pyrazolo[3,4-d]pyrimidine as a white solid (1.9 g).

6-Chloro-1-ethyl-3-iodo-4-morpholin-4-yl-1H-pyrazolo[3,4-d]pyrimidine (200 mg) was dissolved in dimethylformamide (3.0 mL). Copper iodide (19 mg), dichlorobis (triphenylphosphine) palladium, triethylamine (0.465 mL) and propargyl chloride (5.0 eq.) were added. After 3 h the reaction was partitioned between water and ethyl acetate and the insolubles were filtered off. The organic phase was separated and dried with magnesium sulfate then evaporated leaving a crude solid that was purified by silica gel column to give 3-(6-chloro-1-ethyl-4-morpholin-4-yl-1H-pyrazolo[3,4-d]pyrimidin-3-yl)prop-2-yn-1-ol (95 mg).

### Example 60 and 61

### N-(4-{4-(2-hydroxymorpholin-4-yl)-1-[1-(pyridin-3-ylmethyl)piperidin-4-yl]-1H-pyrazolo[3,4-d]pyrimidin-6-yl}phenyl)acetamide and 1-(4-{4-(2-hydroxymorpholin-4-yl)-1-[1-(pyridin-3-ylmethyl)piperidin-4-yl]-1H-pyrazolo[3,4-d]pyrimidin-6-yl}phenyl)-3-methylurea

### Materials:

Pooled nude mouse liver microsomes were purchased from XenoTech (Lenexa, Kans.). NADPH was purchased from BD Gentest (Franklin Lakes, N.J.). HPLC grade water, acetonitrile, ethyl acetate, sodium phosphate dibasic, potassium phosphate monobasic were obtained from EM Science (Gibbstown, N.J.). Ammonium acetate was purchased from Sigma (St. Louis, Mo., USA).

### Microsomal Incubation:

N-(4-{4-morpholin-4-yl-1-[1-(pyridin-3-ylmethyl)piperidin-4-yl]-1H-pyrazolo[3,4-d]pyrimidin-6-yl}phenyl)acetamide and N-methyl-N′-(4-{4-morpholin-4-yl-1-[1-(pyridin-3-ylmethyl)piperidin-4-yl]-1H-pyrazolo[3,4-d]pyrimidin-6-yl}phenyl)urea (50 μM each) were incubated separately with nude mouse liver microsomes (1 mg micromal protein/mL of incubation) and NADPH (1 mM) in potassium phosphate buffer (100 mM, pH 7.4) at 37° C. for 90 min. The total incubation volume for each compound was 50 mL. The incubation reaction was initiated by addition of NADPH after 5 min pre-incubation and was stopped by liquid-liquid extraction using ethyl acetate (incubation solution:ethyl acetate=1:4, v/v). The ethyl acetate layer was separated and evaporated to dryness using a rotary evaporator (Buchi, Postfach, Switzerland). The residue was reconstituted with 1 ml of a water-acetonitrile mixture (10:90, v/v) for HPLC isolation.

### Metabolite Isolation:

A Waters 2790HPLC system (Waters, Beverly, Mass., USA) was used for the isolation of these metabolites. The system consisted of two quaternary pumps, a vacuum degasser, a temperature controlled autosampler, a thermostated column compartment, a fraction collector and a PDA detector. The chromatographic separation was carried out using a Luna C18 column (150×4.6 mm i.d., 5 μM particle size) (Phenomenex, Torrance, Calif.) at an oven temperature of 40° C. The mobile phase consisted of solvent A: 10 mM ammonium acetate in water-acetonitrile (H₂O:ACN=95:5, v/v) and B: 10 mM ammonium acetate in acetonitrile-water (ACN:H₂O=95:5, v/v). The mobile phase gradient started with 20% B, and then increased linearly from 20% to 80% B in 20 min. The flow rate was 1 ml min. The HPLC elution fractions were collected using a Waters fraction collector. The injection volume was 50 μL and 20 injections were performed in total. The fractions of the isolated metabolite from 20 injections were combined, and were dried down using a Savant (Thermo Quest, Holbrook, N.Y.). The purity and identity of metabolites were checked using LC/UV/MS and LC/MS/MS. The structures of these metabolites were determined using NMR.

### Example 62

### 6-(1H-indol-5-yl)-4-morpholin-4-yl-1-[1-(2,2,2-trifluoroethyl)piperidin-4-yl]-1H-pyrazolo[3,4-d]pyrimidine (Scheme 47)

To 6-chloro-4-morpholin-4-yl-1H-pyrazolo[3,4-d]pyrimidine (J, prepared according to scheme 4, 0.72 g, 3.0 mmol), tert-butyl 4-hydroxypiperidine-1-carboxylate (0.63 g, 3.2 mmol), triphenylphosphine (0.87 g, 3.3 mmol) in THF (20 mL) is added DIAD (0.77 mL, 3.9 mmol) dropwise over 5 min. After stirring overnight, the mixture is concentrated in vacuo, dissolved in DMSO and purified by reverse phase HPLC. Alternatively, the crude product is purified by flash chromatography (hexanes/ethyl acetate) to provide tert-butyl 4-(6-chloro-4-morpholino-1H-pyrazolo[3,4-d]pyrimidin-1-yl)piperidine-1-carboxylate as a pale yellow foam.

MS (ES⁺): 423.3 (M+H)⁺

Tert-butyl 4-(6-chloro-4-morpholino-1H-pyrazolo[3,4-d]pyrimidin-1-yl)piperidine-1-carboxylate (0.36 g, 0.85 mmol) is dissolved in dichloromethane (10 mL) and then treated with trifluoroacetic acid (2 mL). The reaction mixture is concentrated to dryness under reduced pressure and then diethyl ether is evaporated from the residue three times to give 4-(6-chloro-1-(piperidin-4-yl)-1H-pyrazolo[3,4-d]pyrimidin-4-yl)morpholine, trifluoroacetic acid salt as a golden solid.

4-(6-Chloro-1-(piperidin-4-yl)-1H-pyrazolo[3,4-d]pyrimidin-4-yl)morpholine, trifluoroacetatic acid salt (0.85 mmol) is taken up in acetone (3 mL) and then treated successively with potassium carbonate (0.47 g, 3.4 mmol) and 2,2,2-trifluoroethanesulfonic acid trichloromethyl ester (0.31 g). The mixture is heated at reflux for 17 hours before being concentrated to dryness to under reduced pressure. The residue is partitioned between ethyl acetate and water. The aqueous phase is extracted with ethyl acetate three times. The combined extracts are washed with a saturated aqueous solution of sodium chloride, dried over anhydrous magnesium sulfate, filtered, and concentrated to dryness under reduced pressure to afford 4-(6-chloro-1-(1-(2,2,2-trifluoroethyl)piperidin-4-yl)-1H-pyrazolo[3,4-d]pyrimidin-4-yl)morpholine.

Crude 4-(6-chloro-1-(1-(2,2,2-trifluoroethyl)piperidin-4-yl)-1H-pyrazolo[3,4-d]pyrimidin-4-yl)morpholine (0.43 mmol max.) is coupled to indole 5-boronic acid (0.082 g, 0.51 mmol) with tetrakis(triphenylphosphine) palladium (0) (5 mol %), 2 M aqueous sodium carbonate solution, and ethylene glycol dimethyl ether (DME), under microwave irradiation (175° C., 10 min). Following an aqueous workup, reverse phase high performance liquid chromatography, employing a Phenomenex Prodigy 250 mm×21.2 mm 5 μm column and a 15% acetonitrile/85% water/0.1% trifluoroacetic acid to 100% acetonitrile gradient over 35 minutes, and concentration, 6-(1H-indol-5-yl)-4-morpholin-4-yl-1-[1-(2,2,2-trifluoroethyl)piperidin-4-yl]-1H-pyrazolo[3,4-d]pyrimidine is obtained as a solid.

MS (ES⁺): 486.2 (M+H)⁺

### Example 63

### 1-methyl-3-(4-{4-morpholin-4-yl-1-[1-(2,2,2-trifluoroethyl)piperidin-4-yl]-1H-pyrazolo[3,4-d]pyrimidin-6-yl}phenyl)urea (Scheme 48)

Crude 4-(6-chloro-1-(1-(2,2,2-trifluoroethyl)piperidin-4-yl)-1H-pyrazolo[3,4-d]pyrimidin-4-yl)morpholine (from example 62, 0.43 mmol max.) is coupled to 4-aminophenylboronic acid pinacol ester (0.11 g, 0.51 mmol) with tetrakis(triphenylphosphine) palladium (0) (5 mol %), 2 M aqueous sodium carbonate solution, and ethylene glycol dimethyl ether (DME), under microwave irradiation (175° C., 10 min). Following an aqueous workup, reverse phase high performance liquid chromatography, employing a Phenomenex Prodigy 250 mm×21.2 mm 5 μm column and a 15% acetonitrile/85% water/0.1% trifluoroacetic acid to 100% acetonitrile gradient over 35 minutes, and concentration, 4-(4-morpholino-1-(1-(2,2,2-trifluoroethyl)piperidin-4-yl)-1H-pyrazolo[3,4-d]pyrimidin-6-yl)aniline is obtained as a solid.

4-(4-morpholino-1-(1-(2,2,2-trifluoroethyl)piperidin-4-yl)-1H-pyrazolo[3,4-d]pyrimidin-6-yl)aniline (90 mg, 0.20 mmol) is taken up in dichloromethane (5 mL) and triethylamine (3 drops). Triphosgene (29 mg, 0.10 mmol) is added and then after 10 minutes, methylamine solution (2.0 M in tetrahydrofuran, 3 mL) is added. The mixture is concentrated to dryness under reduced pressure and the residue is then purified by reverse phase high performance liquid chromatography, employing a Phenomenex Prodigy 250 mm×21.2 mm 5 μm column and a 15% acetonitrile/85% water/0.1% trifluoroacetic acid to 100% acetonitrile gradient over 35 minutes, to obtain 1-methyl-3-(4-{4-morpholin-4-yl-1-[1-(2,2,2-trifluoroethyl)piperidin-4-yl]-1H-pyrazolo[3,4-d]pyrimidin-6-yl}phenyl)urea as a solid (48 mg).

MS (ES⁺): 519.2 (M+H)⁺

### Example 64

### 2-hydroxyethyl (4-{4-morpholin-4-yl-1-[1-(2,2,2-trifluoroethyl)piperidin-4-yl]-1H-pyrazolo[3,4-d]pyrimidin-6-yl}phenyl)carbamate (Scheme 48)

4-(4-morpholino-1-(1-(2,2,2-trifluoroethyl)piperidin-4-yl)-1H-pyrazolo[3,4-d]pyrimidin-6-yl)aniline (0.26 g, 0.56 mmol) is taken up in dichloromethane (5 mL) and is then treated with triethylamine (0.40 mL) and then triphosgene (0.17 g). After 5 minutes, ethylene glycol (0.62 mL) is added. The mixture is concentrated under reduced pressure and the residues purified by reverse phase high performance liquid chromatography, employing a Phenomenex Prodigy 250 mm×21.2 mm 5 μm column and a 5% acetonitrile/85% water/0.1% trifluoroacetic acid to 100% acetonitrile gradient over 40 minutes and/or a Phenomenex Gemini 5 μm C18 AXIA 100 mm×21.2 mm column and a 5% acetonitrile/95% water/0.07% ammonium hydroxide to 65% acetonitrile/35% water/0.07% ammonium hydroxide gradient over 20 minutes, to obtain 2-hydroxyethyl (4-{4-morpholin-4-yl-1-[1-(2,2,2-trifluoroethyl)piperidin-4-yl]-1H-pyrazolo[3,4-d]pyrimidin-6-yl}phenyl)carbamate as a solid.

### Example 65

### methyl 4-[6-{4-[(methoxycarbonyl)amino]phenyl}-4-(2-methylmorpholin-4-yl)-1H-pyrazolo[3,4-d]pyrimidin-1-yl]piperidine-1-carboxylate (Scheme 49)

Crude 2-methylmorpholine (approximately 100 mmol, prepared according to J. Org. Chem., 1946, 11, 286-291) is dissolved in aqueous 1,4-dioxane (1:1, 250 mL) and then treated successively with sodium hydroxide (6 g, 150 mmol) and di-tert-butyl dicarbonate (22 g, 100 mmol). After one hour of stirring at room temperature, the mixture is extracted three times with dichloromethane. The combined extracts are washed with water, dried over anhydrous magnesium sulfate, filtered, and concentrated under reduced pressure to give tert-butyl 2-methylmorpholine-4-carboxylate as a clear, pale straw colored liquid.

Tert-butyl 2-methylmorpholine-4-carboxylate (approximately 3 g) is then dissolved in dichloromethane and treated with trifluoroacetic acid. After 10 minutes, the mixture is concentrated to dryness under reduced pressure. Dichloromethane is added to the residue and evaporated a total of three times. Diethyl ether is then added to the residue to provide the trifluoroacetic acid salt of 2-methylmorpholine as a white solid, which is collected by Büchner filtration and dried under house vacuum

To a solution of the dichloride (10 mmol), dissolved in EtOH (30 mL) and triethylamine (5 mL), is added 2-methylmorpholine trifluoroacetate (3.3 g, 15 mmol), and the reaction is stirred overnight. The mixture is concentrated to dryness under reduced pressure, then purified by flash chromatography (chloroform/methanol) to provide 4-(1-(1-benzylpiperidin-4-yl)-6-chloro-1H-pyrazolo[3,4-d]pyrimidin-4-yl)-2-methylmorpholine.

A solution of 4-(1-(1-benzylpiperidin-4-yl)-6-chloro-1H-pyrazolo[3,4-d]pyrimidin-4-yl)-2-methylmorpholine (1.0 g, 2.3 mmol) in 1,2-dichloroethane (15 mL) is treated successively with potassium carbonate (0.97 g, 7.0 mmol) and methyl chloroformate (0.54 mL, 7.0 mmol). The mixture is stirred at 50° C. for two hours and then filtered to remove the solids. After concentration of the volatiles under reduced pressure, methyl 4-(6-chloro-4-(2-methylmorpholino)-1H-pyrazolo[3,4-d]pyrimidin-1-yl)piperidine-1-carboxylate is provided as a pale yellow foam.

Methyl 4-(6-chloro-4-(2-methylmorpholino)-1H-pyrazolo[3,4-d]pyrimidin-1-yl)piperidine-1-carboxylate (2.3 mmol) is coupled to 4-aminophenylboronic acid pinacol ester (0.76 g, 3.5 mmol) with tetrakis(triphenylphosphine) palladium (0) (5 mol %), 2 M aqueous sodium carbonate solution, and ethylene glycol dimethyl ether (DME), under microwave irradiation (175° C., 12 min). Following an aqueous workup, flash chromatography (chloroform./methanol) provided impure methyl 4-(6-(4-aminophenyl)-4-(2-methylmorpholino)-1H-pyrazolo[3,4-d]pyrimidin-1-yl)piperidine-1-carboxylate as a dark brown foam.

Impure methyl 4-(6-(4-aminophenyl)-4-(2-methylmorpholino)-1H-pyrazolo[3,4-d]pyrimidin-1-yl)piperidine-1-carboxylate (approximately 0.12 mmol) is taken up in dichloromethane (4 mL). Triphosgene (18 mg) is added as a solution in dichloromethane and then after 10 minutes, methylamine solution (2.0 M in tetrahydrofuran, excess) is added. The mixture is concentrated to dryness under reduced pressure and the residue is then purified by reverse phase high performance liquid chromatography, employing a Phenomenex Prodigy 250 mm×21.2 mm 5 μm column and a 5% acetonitrile/95% water/0.1% trifluoroacetic acid to 100% acetonitrile gradient over 30 minutes, to obtain methyl 4-[6-{4-[(methylcarbamoyl)amino]phenyl}-4-(2-methylmorpholin-4-yl)-1H-pyrazolo[3,4-d]pyrimidin-1-yl]piperidine-1-carboxylate as a solid (27 mg).

### Example 66

### 1-(4-{4-[(2R,6S)-2,6-dimethylmorpholin-4-yl]-1-[1-(pyridin-3-ylmethyl)piperidin-4-yl]-1H-pyrazolo[3,4-d]pyrimidin-6-yl}phenyl)-3-methylurea (Scheme 50)

To a solution of the dichloride (4.7 mmol) dissolved in EtOH (30 mL) is added 2,6-cis-dimethylmorpholine (5.4 g, 47 mmol) and the reaction stirred overnight. The mixture is concentrated to dryness under reduced pressure, then purified by flash chromatography (chloroform/methanol) to provide (2S,6R)-4-(1-(1-benzylpiperidin-4-yl)-6-chloro-1H-pyrazolo[3,4-d]pyrimidin-4-yl)-2,6-dimethylmorpholine as a pale yellow foam.

A solution of (2S,6R)-4-(1-(1-benzylpiperidin-4-yl)-6-chloro-1H-pyrazolo[3,4-d]pyrimidin-4-yl)-2,6-dimethylmorpholine (0.64 g, 1.5 mmol) in 1,2-dichloroethane (15 mL) is treated with ACE-Cl (0.73 mL, 6.7 mmol). After five hours, potassium carbonate (600 mg) is added, and on the following day, additional ACE-Cl (0.80 mL) and potassium carbonate (500 mg) are added. The mixture is filtered through a sintered glass funnel, and after concentration, the filtrate is heated to reflux in methanol. After concentration, the mixture is purified by reverse phase high performance liquid chromatography (TFA buffer) to provide (2S,6R)-4-(6-chloro-1-(piperidin-4-yl)-1H-pyrazolo[3,4-d]pyrimidin-4-yl)-2,6-dimethylmorpholine (0.49 g) as its TFA salt. A suspension of 3-pyridine carboxaldehyde (0.17 g, 1.6 mmol) and (2S,6R)-4-(6-chloro-1-(piperidin-4-yl)-1H-pyrazolo[3,4-d]pyrimidin-4-yl)-2,6-dimethylmorpholine (0.48 g, 1.0 mmol) in THF (20 mL) is stirred until the mixture is homogeneous. Sodium (triacetoxy)borohydride (0.33 g, 1.6 mmol) is added and reaction is left to stir overnight. Upon completion of the reaction, the mixture is subjected to an aqueous work-up. The organics are dried over anhydrous magnesium sulfate and concentrated to give (2S,6R)-4-(6-chloro-1-(1-(pyridin-3-ylmethyl)piperidin-4-yl)-1H-pyrazolo[3,4-d]pyrimidin-4-yl)-2,6-dimethylmorpholine (0.41 g).

(2S,6R)-4-(6-chloro-1-(1-(pyridin-3-ylmethyl)piperidin-4-yl)-1H-pyrazolo[3,4-d]pyrimidin-4-yl)-2,6-dimethylmorpholine (0.93 mmol) is coupled to 4-aminophenylboronic acid pinacol ester (0.30 g, 1.4 mmol) with tetrakis(triphenylphosphine) palladium (0) (10 mol %), 2 M aqueous sodium carbonate solution, and ethylene glycol dimethyl ether (DME), under microwave irradiation (180° C., 15 min). Following an aqueous workup, reverse phase high performance liquid chromatography, employing a Phenomenex Prodigy 250 mm×21.2 mm 5 μm column and a 5% acetonitrile/95% water/0.1% trifluoroacetic acid to 100% acetonitrile gradient over 40 minutes, and concentration, 4-(4-((2S,6R)-2,6-dimethylmorpholino)-1-(1-(pyridin-3-ylmethyl)piperidin-4-yl)-1H-pyrazolo[3,4-d]pyrimidin-6-yl)aniline is obtained as is TFA salt. The TFA salt is then partitioned between ethyl acetate and saturated aqueous sodium hydrogen carbonate solution. The organic phase is washed twice with saturated aqueous sodium hydrogen carbonate solution and once with 0.5 M aqueous sodium hydroxide solution. After drying the organics over anhydrous magnesium sulfate and concentration, the desired compound is obtained as a free base.

### Example 67

### 3-(4-morpholino-1-(2,2,2-trifluoroethyl)-1H-pyrazolo[3,4-d]pyrimidin-6-yl)phenol (Scheme 55)

To 6-chloro-4-morpholin-4-yl-1H-pyrazolo[3,4-d]pyrimidine (1.2 g, 5.0 mmol), potassium carbonate (2.1 g, 15 mmol) and 2,2,2-trifluoroiodoethane (2.0 mL, 20 mmol) is added N,N-dimethylformamide (50 mL). The reaction mixture is heated in a sealed tube at 80° C. for 18 hours. After an aqueous work-up, reverse phase HPLC gives 4-(6-chloro-1-(2,2,2-trifluoroethyl)-1H-pyrazolo[3,4-d]pyrimidin-4-yl)morpholine as a solid (450 mg).

4-(6-chloro-1-(2,2,2-trifluoroethyl)-1H-pyrazolo[3,4-d]pyrimidin-4-yl)morpholine (0.19 g, 0.59 mmol) is coupled to 3-hydroxyphenylboronic acid (0.89 mmol) with tetrakis(triphenylphosphine) palladium (0) (10 mol %), 2 M aqueous sodium carbonate solution, and ethylene glycol dimethyl ether (DME), under microwave irradiation (175° C., 10 min). Following an aqueous workup, reverse phase high performance liquid chromatography, employing a Phenomenex Prodigy 250 mm×21.2 mm 5 μm column and a 15% acetonitrile/85% water/0.1% trifluoroacetic acid to 100% acetonitrile gradient over 40 minutes, and concentration, 3-(4-morpholino-1-(2,2,2-trifluoroethyl)-1H-pyrazolo[3,4-d]pyrimidin-6-yl)phenol is obtained as a solid.

### Example 68

### 1-methyl-3-(4-(4-morpholino-1-(2,2,2-trifluoroethyl)-1H-pyrazolo[3,4-d]pyrimidin-6-yl)phenyl)urea (Scheme 56)

4-(6-chloro-1-(2,2,2-trifluoroethyl)-1H-pyrazolo[3,4-d]pyrimidin-4-yl)morpholine (0.26 g, 0.81 mmol) is coupled to 4-aminophenylboronic acid, pinacol ester (0.27 g, 1.2 mmol) with tetrakis(triphenylphosphine) palladium (0) (10 mol %), 2 M aqueous sodium carbonate solution, and ethylene glycol dimethyl ether (DME), under microwave irradiation (180° C., 20 min). Following an aqueous workup, reverse phase high performance liquid chromatography, employing a Phenomenex Prodigy 250 mm×21.2 mm 5 μm column and a 15% acetonitrile/85% water/0.1% trifluoroacetic acid to 100% acetonitrile gradient over 40 minutes, and concentration, 4-(4-morpholino-1-(2,2,2-trifluoroethyl)-1H-pyrazolo[3,4-d]pyrimidin-6-yl)aniline is obtained as a solid (142 mg).

4-(4-morpholino-1-(2,2,2-trifluoroethyl)-1H-pyrazolo[3,4-d]pyrimidin-6-yl)aniline (32 mg, 0.08 mmol) is taken up in dichloromethane (5 mL). Triphosgene (13 mg, 0.04 mmol) is added and then after 10 minutes, methylamine solution (2.0 M in tetrahydrofuran, 0.43 mL) is added. The mixture is concentrated to dryness under reduced pressure and the residue is then purified by reverse phase high performance liquid chromatography, employing a Phenomenex Gemini 100 mm×21.2 mm 5 μm column and a 5% acetonitrile/95% water/0.1% trifluoroacetic acid to 100% acetonitrile gradient over 25 minutes, to obtain 1-methyl-3-(4-(4-morpholino-1-(2,2,2-trifluoroethyl)-1H-pyrazolo[3,4-d]pyrimidin-6-yl)phenyl)urea as a solid (11 mg).

### Example 69

3-(4-((2S,6R)-2,6-dimethylmorpholino)-1-phenyl-1H-pyrazolo[3,4-d]pyrimidin-6-yl)phenol (R₁₅ is hydrogen, optionally substituted C₁-C₆alkyl, optionally substituted C₃-C₈carbocycle, optionally substituted C₆-C₁₄aryl, optionally substituted C₁-C₉heteroaryl, optionally substituted (C₁-C₆alkyl)amino, or optionally substituted (C₆-C₁₄aryl)amino, with the proviso that when R₁₅ is in —SO₂—R₁₅, R₁₅ is not —H; )

5-amino-1-phenyl-1H-pyrazole-4-carbonitrile (1.7 g, 9.2 mmol, prepared according to J. Med. Chem., 1991, 34, 2892-98) is taken up with triethylamine (4.7 mL, 36 mmol) in dichloromethane (50 mL) and cooled in an ice-water bath. 3-Anisoyl chloride (2.0 g, 12 mmol) is added and the cooling bath is removed. 4-Dimethylaminopyridine (4-DMAP, 100 mg) is added and the vessel is heated to reflux for 20 minutes. Following removal of the volatiles under reduced pressure, the residue is partitioned between ethyl acetate and water. The aqueous phase is extracted with ethyl acetate. The combined extracts are washed with saturated aqueous sodium hydrogen carbonate solution, water, 5% aqueous potassium hydrogen sulfate, and saturated aqueous sodium chloride solution. Following drying over anhydrous magnesium sulfate and concentrated under reduced pressure, N-(4-cyano-1-phenyl-1H-pyrazol-5-yl)-3-methoxybenzamide is obtained as a white foam.

N-(4-cyano-1-phenyl-1H-pyrazol-5-yl)-3-methoxybenzamide (approx 10 g, 31 mmol) is suspended in aqueous ethanol (1:1, 200 mL). Hydrogen peroxide solution (30% in water, 10 mL) and sodium hydroxide (2 g) are added. The mixture is heated for 15 minutes at 50° C. and then at reflux overnight. Additional hydrogen peroxide solution (20 mL) and sodium hydroxide (3 g) are added. The mixture is again heated for 15 minutes at 50° C. and then at reflux overnight. The mixture is allowed to cool to room temperature. Glacial acetic acid or concentrated hydrochloric acid is added to precipitate 6-(3-methoxyphenyl)-1-phenyl-1H-pyrazolo[3,4-d]pyrimidin-4-ol, which is washed with water and dried under house vacuum and then over phosphorus pentoxide (P₂O₅) in a vacuum oven.

6-(3-methoxyphenyl)-1-phenyl-1H-pyrazolo[3,4-d]pyrimidin-4-ol (2.0 g, 6.3 mmol) is suspended in phosphorous oxychloride (POCl₃, 20 mL) in a sealed tube. The mixture is heated until the solid until the solid is completely dissolved, then the mixture is allowed to cool to room temperature. After concentration of the mixture to dryness under reduced pressure, the crude 4-chloro-6-(3-methoxyphenyl)-1-phenyl-1H-pyrazolo[3,4-d]pyrimidine is taken up in dichloromethane (100 mL) and cooled in an ice-water bath. Boron tribromide solution (1.0 M in dichloromethane) is added to the mixture, which is then allowed to regain room temperature. The mixture is concentrated under reduced pressure and quenched by the addition of saturated aqueous sodium hydrogen carbonate solution. The mother liquor is extracted with ethyl acetate. The combined extracts are washed with saturated aqueous sodium chloride solution, dried over anhydrous magnesium sulfate, and concentrated under reduced pressure to provide 3-(4-bromo-1-phenyl-1H-pyrazolo[3,4-d]pyrimidin-6-yl)phenol.

A suspension of 3-(4-bromo-1-phenyl-1H-pyrazolo[3,4-d]pyrimidin-6-yl)phenol (50 mg) in ethanol (10 mL) is treated with 10 equivalents of cis 2,6-dimethylmorpholine. The mixture is heated for 10 minutes in a 60° C. oil bath. After concentrated of the mixture under reduced pressure, the residue is dissolved in dimethylsulfoxide (DMSO)/methanol and purified by reverse phase high performance liquid chromatography to provide 3-(4-((2S,6R)-2,6-dimethylmorpholino)-1-phenyl-1H-pyrazolo[3,4-d]pyrimidin-6-yl)phenol.

### Example 70

### (S)-3-(4-(3-methylmorpholino)-1-phenyl-1H-pyrazolo[3,4-d]pyrimidin-6-yl)phenol (Scheme 52)

A suspension of 3-(4-bromo-1-phenyl-1H-pyrazolo[3,4-d]pyrimidin-6-yl)phenol (50 mg) and 3-(S)-methylmorpholine (50 mg) in ethanol (1.5 mL) is heated for 5 minutes at 185° C. in the microwave reactor. After concentrated of the mixture under reduced pressure, the residue is dissolved in dimethylsulfoxide (DMSO)/methanol and purified by reverse phase high performance liquid chromatography to provide (S)-3-(4-(3-methylmorpholino)-1-phenyl-1H-pyrazolo[3,4-d]pyrimidin-6-yl)phenol.

### Example 71

### 3-{4-[(3R)-3-methylmorpholin-4-yl]-1-phenyl-1H-pyrazolo[3,4-d]pyrimidin-6-yl}phenol (Scheme 57)

A suspension of 3-(4-bromo-1-phenyl-1H-pyrazolo[3,4-d]pyrimidin-6-yl)phenol (180 mg) and 3-(R)-methylmorpholine (61 mg) in ethanol (3 mL) is heated for 12 minutes at 180° C. in the microwave reactor. After concentrated of the mixture under reduced pressure, the residue is dissolved in dimethylsulfoxide (DMSO)/methanol and purified by reverse phase high performance liquid chromatography to provide 3-{4-[(3R)-3-methylmorpholin-4-yl]-1-phenyl-1H-pyrazolo[3,4-d]pyrimidin-6- yl}phenol.

### Example 72

### 3-[4-(2-methylmorpholin-4-yl)-1-phenyl-1H-pyrazolo[3,4-d]pyrimidin-6-yl]phenol (Scheme 58)

A suspension of 3-(4-bromo-1-phenyl-1H-pyrazolo[3,4-d]pyrimidin-6-yl)phenol (150 mg, 1.0 mmol) and 2-methylmorpholine trifluoroacetate (1.2 mmol) in ethanol (5 mL) and triethylamine (1 mL) is heated for 10 minutes at 180° C. in the microwave reactor. After concentrated of the mixture under reduced pressure, the residue is dissolved in dimethylsulfoxide (DMSO)/methanol and purified by reverse phase high performance liquid chromatography to provide 3-[4-(2-methylmorpholin-4-yl)-1-phenyl-1H-pyrazolo[3,4-d]pyrimidin-6-yl]phenol.

### Example 73

### 4-(6-(3-hydroxyphenyl)-1-phenyl-1H-pyrazolo[3,4-d]pyrimidin-4-yl)morpholin-3-one (Scheme 53)

A mixture of 4-chloro-6-(3-methoxyphenyl)-1-phenyl-1H-pyrazolo[3,4-d]pyrimidine (0.21 g, 0.61 mmol), morpholin-3-one (75 mg, 0.74 mmol), cesium carbonate (0.28 g, 0.86 mmol), tris(dibenzylideneacetone)dipalladium(0) (Pd₂dba₃, 2.8 mg, 0.5 mol %), and 4,5-bis(diphenylphosphino)-9,9-dimethylxanthene (Xant phos, 5.3 mg, 1.5 mol %) in 1,4-dioxane (1.5 mL) is heated in a microwave reactor for 20 minutes at 120° C. Water is added to the mixture; the solids are collected by filtration and dried under house vacuum to give 4-(6-(3-methoxyphenyl)-1-phenyl-1H-pyrazolo[3,4-d]pyrimidin-4-yl)morpholin-3-one as a light gray solid (208 mg).

4-(6-(3-methoxyphenyl)-1-phenyl-1H-pyrazolo[3,4-d]pyrimidin-4-yl)morpholin-3-one (0.15 g, 0.37 mmol) is taken up in dichloromethane and the mixture is cooled in an ice-water bath. Boron tribromide solution (1.0 M in dichloromethane, 2 mL) is added to the mixture, which is then allowed to regain room temperature. The mixture is concentrated under reduced pressure. The solid residue is taken up in water and dichloromethane and collected by filtration. The solid are dissolved in 10% methanol/dichloromethane. The solution of solid is combined with the water and dichloromethane filtrates. The aqueous phase is extracted with dichloromethane. The combined organics are dried over anhydrous magnesium sulfate, filtered, and concentrated to a solid under reduced pressure. The crude solid is dissolved in DMSO/methanol and purified by reverse phase high performance liquid chromatography to provide 4-(6-(3-hydroxyphenyl)-1-phenyl-1H-pyrazolo[3,4-d]pyrimidin-4-yl)morpholin-3-one.

### Example 74

### 4-(6-(3-hydroxyphenyl)-1-phenyl-1H-pyrazolo[3,4-d]pyrimidin-4-yl)morpholin-3-one (Scheme 54)

A mixture of 3-(4-bromo-1-phenyl-1H-pyrazolo[3,4-d]pyrimidin-6-yl)phenol

(70 mg, 0.19 mmol), morpholin-3-one (23 mg, 0.23 mmol), cesium carbonate (87 mg, 0.27 mmol), tris(dibenzylideneacetone)dipalladium(0) (Pd₂dba₃, 0.87 mg, 0.5 mol %), and 4,5-bis(diphenylphosphino)-9,9-dimethylxanthene (Xant phos, 1.6 mg, 1.5 mol %) in 1,4-dioxane (0.5 mL) is heated in a microwave reactor for 20 minutes at 120° C. Water is added to the mixture and is then extracted with dichloromethane. The extracts are dried over anhydrous magnesium sulfate, filtered, and concentrated under reduced pressure to provide 4-(6-(3-hydroxyphenyl)-1-phenyl-1H-pyrazolo[3,4-d]pyrimidin-4-yl)morpholin-3-one.

### Example 75

### 3-[4-(1,4-oxazepan-4-yl)-1-phenyl-1H-pyrazolo[3,4-d ]pyrimidin-6-yl]phenol (Scheme 59)

A suspension of 3-(4-bromo-1-phenyl-1H-pyrazolo[3,4-d]pyrimidin-6-yl)phenol (180 mg, 0.50 mmol) and homomorpholine hydrochloride (83 mg, 0.60 mmol) in ethanol (4 mL) and triethylamine (0.16 mL) is heated for 12 minutes at 180° C. in the microwave reactor. After concentrated of the mixture under reduced pressure, the residue is dissolved in dimethylsulfoxide (DMSO)/methanol and purified by reverse phase high performance liquid chromatography to provide 3-[4-(1,4-oxazepan-4-yl)-1-phenyl-1H-pyrazolo[3,4-d]pyrimidin-6-yl]phenol (100 mg) as a solid.

### Example 76

### 3-(1-phenyl-4-thiomorpholin-4-yl-1H-pyrazolo[3,4-d]pyrimidin-6-yl)phenol (Scheme 60)

A suspension of 3-(4-bromo-1-phenyl-1H-pyrazolo[3,4-d]pyrimidin-6-yl)phenol (65 mg, 0.18 mmol) and thiomorpholine hydrochloride (100 mg) in ethanol (1.5 mL) is heated for 5 minutes at 180° C. in the microwave reactor. After concentrated of the mixture under reduced pressure, the residue is dissolved in dimethylsulfoxide (DMSO)/methanol and purified by reverse phase high performance liquid chromatography to provide 3-(1-phenyl-4-thiomorpholin-4-yl-1H-pyrazolo[3,4-d]pyrimidin-6-yl)phenol (65 mg) as a solid.

### Example 77

### 3-(3-fluoro-4-morpholin-4-yl-1-phenyl-1H-pyrazolo[3,4-d]pyrimidin-6-yl)phenol (Scheme 61)

5-amino-3-fluoro-1-phenyl-1H-pyrazole-4-carbonitrile (prepared via a procedure based on WO 87/03781, 0.38 g, 1.9 mmol) is suspended in dichloromethane (10 mL) and treated with 3-anisoyl chloride (0.42 g, 2.4 mmol), followed by triethylamine (0.75 mL) and 4-DMAP (10 mg). After 30 minutes, additional quantities of 3-anisoyl chloride (200 mg) and triethylamine (0.75 mL) are added. The mixture is heated at reflux for 30 minutes and then is concentrated to dryness under reduced pressure. The residue is treated with pyridine (8 mL), water (1 mL), and 37% ammonium hydroxide solution (6 mL). Following concentration under reduced pressure, the residue is purified by reverse phase high performance liquid chromatography to provide, after concentration, N-(4-cyano-3-fluoro-1-phenyl-1H-pyrazol-5-yl)-3-methoxybenzamide as a light tan foam (230 mg).

A solution of N-(4-cyano-3-fluoro-1-phenyl-1H-pyrazol-5-yl)-3-methoxybenzamide (0.23 g, 0.68 mmol) in aqueous ethanol (1:1, 100 mL) is treated successively with sodium hydroxide (160 mg) and hydrogen peroxide aqueous solution (30%, 0.56 mL). The mixture is heated at 45° C. until the evolution of gases subsides and then at reflux for 30 minutes. The mixture is cooled to room temperature and treated with glacial acetic acid. The precipitate is collected by filtration, washed with water, and dried under house vacuum to provide 3-fluoro-6-(3-methoxyphenyl)-1-phenyl-1H-pyrazolo[3,4-d]pyrimidin-4-ol (200 mg) as a pale, peach colored solid.

3-fluoro-6-(3-methoxyphenyl)-1-phenyl-1H-pyrazolo[3,4-d]pyrimidin-4-ol (0.20 g, 0.59 mmol) is suspended in phosphorous oxychloride (POCl₃, 2 mL) in a Smith process vial. The mixture is heated in a microwave reactor for 10 minutes at 150° C. After concentration of the mixture to dryness under reduced pressure, the crude 4-chloro-3-fluoro-6-(3-methoxyphenyl)-1-phenyl-1H-pyrazolo[3,4-d]pyrimidine is taken up in dichloromethane (5 mL) and cooled in an ice-water bath. Boron tribromide solution (1.0 M in dichloromethane, 5 mL) is added to the mixture, which is then allowed to regain room temperature. The mixture is concentrated under reduced pressure and quenched by the addition of saturated aqueous sodium hydrogen carbonate solution. The mother liquor is extracted with ethyl acetate. The combined extracts are washed with saturated aqueous sodium chloride solution, dried over anhydrous magnesium sulfate, and concentrated under reduced pressure to provide 3-(4-bromo-3-fluoro-1-phenyl-1H-pyrazolo[3,4-d]pyrimidin-6-yl)phenol (230 mg) as a brown solid.

A suspension of 3-(4-bromo-3-fluoro-1-phenyl-1H-pyrazolo[3,4-d]pyrimidin-6-yl)phenol (0.59 mmol) in ethanol is treated with morpholine (0.20 mL). The mixture is heated with a heat gun until it is clear and homogeneous. After concentration of the mixture under reduced pressure, the residue is dissolved in dimethylsulfoxide (DMSO)/methanol and purified by reverse phase high performance liquid chromatography to provide 3-(3-fluoro-4-morpholino-1-phenyl-1H-pyrazolo[3,4-d]pyrimidin-6-yl)phenol.

The following compounds were prepared according to the above procedures:

| | | | | Synthetic |
|---|---|---|---|---|
| | | MS | Retention | Method |
| Compound | Compound Name | (M + H) | Time | (Scheme) |
| 1 | 1-methyl-4-morpholin-4-yl-6-pyridin-3-yl-1H- | 297.3 | 1.72 | 2 |
| | pyrazolo[3,4-d]pyrimidine | | | |
| 2 | 6-(1-benzothien-2-yl)-1-methyl-4-morpholin-4-yl-1H- | 352.4 | 2.71 | 2 |
| | pyrazolo[3,4-d]pyrimidine | | | |
| 3 | 6-(3-furyl)-1-methyl-4-morpholin-4-yl-1H-pyrazolo[3,4- | 286.3 | 2.13 | 2 |
| | d]pyrimidine | | | |
| 4 | 6-(6-methoxypyridin-3-yl)-4-morpholin-4-yl-1-phenyl- | 389.2 | 2.72 | 2 |
| | 1H-pyrazolo[3,4-d]pyrimidine | | | |
| 5 | 4-morpholin-4-yl-1-phenyl-6-pyrimidin-5-yl-1H- | 360.1 | 2.44 | 2 |
| | pyrazolo[3,4-d]pyrimidine | | | |
| 6 | 4-morpholin-4-yl-1-phenyl-6-(1H-pyrrol-2-yl)-1H- | 347.2 | 2.42 | 2 |
| | pyrazolo[3,4-d]pyrimidine | | | |
| 7 | 6-(1-benzothien-3-yl)-4-morpholin-4-yl-1-phenyl-1H- | 414.1 | 2.94 | 2 |
| | pyrazolo[3,4-d]pyrimidine | | | |
| 8 | 3-[1-(1-benzylpiperidin-4-yl)-4-morpholin-4-yl-1H- | 471.2 | 1.97 | 3 |
| | pyrazolo[3,4-d]pyrimidin-6-yl]phenol | | | |
| 9 | 3-(4-morpholin-4-yl-1-piperidin-4-yl-1H-pyrazolo[3,4- | 381.2 | 1.74 | 3 |
| | d]pyrimidin-6-yl)phenol | | | |
| 10 | 3-[1-(1-acetylpiperidin-4-yl)-4-morpholin-4-yl-1H- | 423.2 | 1.98 | 3 |
| | pyrazolo[3,4-d]pyrimidin-6-yl]phenol | | | |
| 11 | 6-(5-methoxypyridin-3-yl)-1-methyl-4-morpholin-4-yl- | 327.1 | 2.10 | 2 |
| | 1H-pyrazolo[3,4-d]pyrimidine | | | |
| 12 | 1-[1-(1-methyl-6-phenyl-1H-pyrazolo[3,4-d]pyrimidin-4- | 426.2 | 2.42 | 2 |
| | yl)piperidin-4-yl]-1,3-dihydro-2H-benzimidazol-2-one | | | |
| 13 | 3-{1-[1-(cyclopropylmethyl)piperidin-4-yl]-4-morpholin- | 435.2 | 1.86 | 3 |
| | 4-yl-1H-pyrazolo[3,4-d]pyrimidin-6-yl}phenol | | | |
| 14 | 3-{4-morpholin-4-yl-1-[1-(1H-pyrrol-2- | 460.2 | 1.88 | 3 |
| | ylmethyl)piperidin-4-yl]-1H-pyrazolo[3,4-d]pyrimidin-6- | | | |
| | yl}phenol | | | |
| 15 | 3-{1-[1-(2-furylmethyl)piperidin-4-yl]-4-morpholin-4-yl- | 461.2 | 1.90 | 3 |
| | 1H-pyrazolo[3,4-d]pyrimidin-6-yl}phenol | | | |
| 16 | 3-{4-morpholin-4-yl-1-[1-(pyridin-3-ylmethyl)piperidin- | 472.2 | 1.76 | 3 |
| | 4-yl]-1H-pyrazolo[3,4-d]pyrimidin-6-yl}phenol | | | |
| 17 | 3-{1-[1-(cyclohexylmethyl)piperidin-4-yl]-4-morpholin- | 477.3 | 2.03 | 3 |
| | 4-yl-1H-pyrazolo[3,4-d]pyrimidin-6-yl}phenol | | | |
| 18 | 3-(1-{1-[(5-methyl-2-thienyl)methyl]piperidin-4-yl}-4- | 491.2 | 2.02 | 3 |
| | morpholin-4-yl-1H-pyrazolo[3,4-d]pyrimidin-6-yl)phenol | | | |
| 19 | 3-{1-[1-(4-chlorobenzyl)piperidin-4-yl]-4-morpholin-4- | 505.2 | 2.06 | 3 |
| | yl-1H-pyrazolo[3,4-d]pyrimidin-6-yl}phenol | | | |
| 20 | 5-({4-[6-(3-hydroxyphenyl)-4-morpholin-4-yl-1H- | 517.2 | 1.88 | 3 |
| | pyrazolo[3,4-d]pyrimidin-1-yl]piperidin-1-yl}methyl)-2- | | | |
| | methoxyphenol | | | |
| 21 | 3-{1-[1-(cyclopropylcarbonyl)piperidin-4-yl]-4- | 449.2 | 2.07 | 3 |
| | morpholin-4-yl-1H-pyrazolo[3,4-d]pyrimidin-6- | | | |
| | yl}phenol | | | |
| 22 | 3-{1-[1-(2-furoyl)piperidin-4-yl]-4-morpholin-4-yl-1H- | 475.6 | 2.01 | 3 |
| | pyrazolo[3,4-d]pyrimidin-6-yl}phenol | | | |
| 23 | 3-(1-{1-[(6-chloropyridin-3-yl)carbonyl]piperidin-4-yl}- | 520.2 | 2.13 | 3 |
| | 4-morpholin-4-yl-1H-pyrazolo[3,4-d]pyrimidin-6- | | | |
| | yl)phenol | | | |
| 24 | 3-{4-morpholin-4-yl-1-[1-(pyridin-3- | 486.8 | 2.20 | 3 |
| | ylcarbonyl)piperidin-4-yl]-1H-pyrazolo[3,4-d]pyrimidin- | | | |
| | 6-yl}phenol | | | |
| 25 | 3-[1-(1-benzoylpiperidin-4-yl)-4-morpholin-4-yl-1H- | 485.6 | 2.32 | 3 |
| | pyrazolo[3,4-d]pyrimidin-6-yl]phenol | | | |
| 26 | 3-{1-[1-(cyclohexylcarbonyl)piperidin-4-yl]-4-morpholin- | 491.6 | 2.32 | 3 |
| | 4-yl-1H-pyrazolo[3,4-d]pyrimidin-6-yl}phenol | | | |
| 27 | 3-{1-[1-(4-chlorobenzoyl)piperidin-4-yl]-4-morpholin-4- | 519.2 | 2.32 | 3 |
| | yl-1H-pyrazolo[3,4-d]pyrimidin-6-yl}phenol | | | |
| 28 | 3-{1-[1-(4-methoxybenzoyl)piperidin-4-yl]-4-morpholin- | 515.2 | 2.22 | 3 |
| | 4-yl-1H-pyrazolo[3,4-d]pyrimidin-6-yl}phenol | | | |
| 29 | 3-[1-(1-isobutyrylpiperidin-4-yl)-4-morpholin-4-yl-1H- | 451.6 | 2.20 | 3 |
| | pyrazolo[3,4-d]pyrimidin-6-yl]phenol | | | |
| 30 | 3-{1-[1-(methoxyacetyl)piperidin-4-yl]-4-morpholin-4-yl- | 453.2 | 2.16 | 3 |
| | 1H-pyrazolo[3,4-d]pyrimidin-6-yl}phenol | | | |
| 31 | 2-[1-(1-benzylpiperidin-4-yl)-4-morpholin-4-yl-1H- | 471.2 | 2.12 | 3 |
| | pyrazolo[3,4-d]pyrimidin-6-yl]phenol | | | |
| 32 | 1-(1-benzylpiperidin-4-yl)-6-(3-methoxyphenyl)-4- | 485.3 | 3.01 | 3 |
| | morpholin-4-yl-1H-pyrazolo[3,4-d]pyrimidine | | | |
| 33 | 6-(1,3-benzodioxol-5-yl)-1-(1-benzylpiperidin-4-yl)-4- | 499.6 | 2.80 | 3 |
| | morpholin-4-yl-1H-pyrazolo[3,4-d]pyrimidine | | | |
| 34 | 1-(1-benzylpiperidin-4-yl)-6-(1H-indol-2-yl)-4- | 495.3 | 2.14 | 3 |
| | morpholin-4-yl-1H-pyrazolo[3,4-d]pyrimidine | | | |
| 35 | 1-(1-benzylpiperidin-4-yl)-4-morpholin-4-yl-6-(2- | 505.3 | 3.33 | 3 |
| | naphthyl)-1H-pyrazolo[3,4-d]pyrimidine | | | |
| 36 | 1-(1-benzylpiperidin-4-yl)-4-morpholin-4-yl-6-(1- | 505.6 | 2.25 | 3 |
| | naphthyl)-1H-pyrazolo[3,4-d]pyrimidine | | | |
| 37 | 3-[1-(1-benzylpiperidin-4-yl)-4-morpholin-4-yl-1H- | 499.2 | 1.90 | 3 |
| | pyrazolo[3,4-d]pyrimidin-6-yl]benzoic acid | | | |
| 38 | 6-(1H-indol-5-yl)-4-morpholin-4-yl-1-[1-(pyridin-3- | 509.3 | 2.25 | 3 |
| | ylcarbonyl)piperidin-4-yl]-1H-pyrazolo[3,4-d]pyrimidine | | | |
| 39 | 6-(1H-indol-5-yl)-4-morpholin-4-yl-1-[1-(pyridin-3- | 495.6 | 2.30 | 3 |
| | ylmethyl)piperidin-4-yl]-1H-pyrazolo[3,4-d]pyrimidine | | | |
| 40 | 6-(1H-indol-5-yl)-1-[1-(methoxyacetyl)piperidin-4-yl]-4- | 476.2 | 2.17 | 3 |
| | morpholin-4-yl-1H-pyrazolo[3,4-d]pyrimidine | | | |
| 41 | 2-{4-morpholin-4-yl-1-[1-(pyridin-3- | 486.2 | 2.39 | 3 |
| | ylcarbonyl)piperidin-4-yl]-1H-pyrazolo[3,4-d]pyrimidin- | | | |
| | 6-yl}phenol | | | |
| 42 | 4-morpholin-4-yl-6-(2-naphthyl)-1-[1-(pyridin-3- | 520.6 | 2.30 | 3 |
| | ylcarbonyl)piperidin-4-yl]-1H-pyrazolo[3,4-d]pyrimidine | | | |
| 43 | 6-(3-methoxyphenyl)-4-morpholin-4-yl-1-[1-(pyridin-3- | 500.6 | 2.33 | 3 |
| | ylcarbonyl)piperidin-4-yl]-1H-pyrazolo[3,4-d]pyrimidine | | | |
| 44 | 4-morpholin-4-yl-6-(2-naphthyl)-1-piperidin-4-yl-1H- | 415.2 | 2.08 | 3 |
| | pyrazolo[3,4-d]pyrimidine | | | |
| 45 | 1-(1-benzylpiperidin-4-yl)-6-(1H-indol-5-yl)-4- | 494.3 | 2.05 | 2 |
| | morpholin-4-yl-1H-pyrazolo[3,4-d]pyrimidine | | | |
| 46 | 6-(1H-indol-5-yl)-4-morpholin-4-yl-1-piperidin-4-yl-1H- | 404.2 | 2.38 | 3 |
| | pyrazolo[3,4-d]pyrimidine | | | |
| 47 | 6-(1H-indol-5-yl)-4-morpholin-4-yl-1-[1-(pyridin-3- | 495.2 | | 3 |
| | ylmethyl)piperidin-4-yl]-1H-pyrazolo[3,4-d]pyrimidine | | | |
| 48 | 6-(1H-indol-5-yl)-1-[1-(3-methoxybenzoyl)piperidin-4- | 538.2 | | 3 |
| | yl]-4-morpholin-4-yl-1H-pyrazolo[3,4-d]pyrimidine | | | |
| 49 | 1-(1-benzylpiperidin-4-yl)-6-(1H-indol-6-yl)-4- | 494.3 | | 2 |
| | morpholin-4-yl-1H-pyrazolo[3,4-d]pyrimidine | | | |
| 50 | 6-(1H-indol-5-yl)-1-(1-isonicotinoylpiperidin-4-yl)-4- | 509.2 | | 3 |
| | morpholin-4-yl-1H-pyrazolo[3,4-d]pyrimidine | | | |
| 51 | 6-(1H-indol-5-yl)-4-morpholin-4-yl-1-[1-(pyridin-2- | 509.2 | 2.4 | 3 |
| | ylcarbonyl)piperidin-4-yl]- | | | |
| | 1H-pyrazolo[3,4-d]pyrimidine | | | |
| 52 | 6-(1H-indol-5-yl)-4-morpholin-4-yl-1-{1-[3- | 576.3 | | 3 |
| | (trifluoromethyl)benzoyl]piperidin-4- | | | |
| | yl}-1H-pyrazolo[3,4-d]pyrimidine | | | |
| 53 | 6-(1H-indol-5-yl)-1-[1-(4-methylbenzoyl)piperidin-4-yl]- | 522.4 | | 3 |
| | 4-morpholin-4-yl-1H-pyrazolo[3,4-d]pyrimidine | | | |
| 54 | 1-[1-(4-fluorobenzoyl)piperidin-4-yl]-6-(1H-indol-5-yl)- | 526.4 | | 3 |
| | 4-morpholin-4-yl-1H-pyrazolo[3,4-d]pyrimidine | | | |
| 55 | 1-[1-(4-fluorobenzyl)piperidin-4-yl]-6-(1H-indol-5-yl)-4- | 512.2 | | |
| | morpholin-4-yl-1H-pyrazolo[3,4-d]pyrimidine | | | |
| 56 | 6-(1H-indol-5-yl)-1-[1-(4-methylbenzyl)piperidin-4-yl]-4- | 508.2 | | 3 |
| | morpholin-4-yl-1H-pyrazolo[3,4-d]pyrimidine | | | |
| 57 | 1-[1-(2-chlorobenzyl)piperidin-4-yl]-6-(1H-indol-5-yl)-4- | 528.2 | | 3 |
| | morpholin-4-yl-1H-pyrazolo[3,4-d]pyrimidine | | | |
| 58 | 4-({4-[6-(1H-indol-5-yl)-4-morpholin-4-yl-1H- | 537.3 | | 3 |
| | pyrazolo[3,4-d]pyrimidin-1-yl]piperidin-1-yl}methyl)- | | | |
| | N,N-dimethylaniline | | | |
| 59 | 1-(1-benzylpiperidin-4-yl)-6-(1H-indol-4-yl)-4- | 494.3 | | 2 |
| | morpholin-4-yl-1H-pyrazolo[3,4-d]pyrimidine | | | |
| 60 | 3-[1-(1-benzylpiperidin-4-yl)-4-morpholin-4-yl-1H- | 470.3 | 1.8 | 2 |
| | pyrazolo[3,4-d]pyrimidin-6-yl]aniline | | | |
| 61 | 3-[1-(1-benzylpiperidin-4-yl)-4-morpholin-4-yl-1H- | 498.3 | 1.97 | 2 |
| | pyrazolo[3,4-d]pyrimidin-6-yl]-N,N-dimethylaniline | | | |
| 62 | 3-[1-(1-benzylpiperidin-4-yl)-4-morpholin-4-yl-1H- | 542.3 | 2.04 | 2 |
| | pyrazolo[3,4-d]pyrimidin-6-yl]-N-methoxy-N- | | | |
| | methylbenzamide | | | |
| 63 | 3-[1-(1-benzylpiperidin-4-yl)-4-morpholin-4-yl-1H- | 528.3 | 1.94 | |
| | pyrazolo[3,4-d]pyrimidin-6-yl]-N-methoxybenzamide | | | |
| 64 | 1-(1-benzylpiperidin-4-yl)-4-morpholin-4-yl-6-pyridin-3- | 456.2 | 1.78 | 2 |
| | yl-1H-pyrazolo[3,4-d]pyrimidine | | | |
| 65 | 1-(1-benzylpiperidin-4-yl)-4-morpholin-4-yl-6-pyridin-4- | 456.2 | 1.71 | 2 |
| | yl-1H-pyrazolo[3,4-d]pyrimidine | | | |
| 66 | 1-(1-benzylpiperidin-4-yl)-6-dibenzo[b,d]furan-4-yl-4- | 545.3 | 2.4 | 2 |
| | morpholin-4-yl-1H-pyrazolo[3,4-d]pyrimidine | | | |
| 67 | 6-(1-benzothien-2-yl)-1-(1-benzylpiperidin-4-yl)-4- | 511.2 | 2.34 | 2 |
| | morpholin-4-yl-1H-pyrazolo[3,4-d]pyrimidine | | | |
| 68 | 6-(1-benzofuran-2-yl)-1-(1-benzylpiperidin-4-yl)-4- | 495.2 | 2.2 | 2 |
| | morpholin-4-yl-1H-pyrazolo[3,4-d]pyrimidine | | | |
| 69 | 3-[1-(1-benzylpiperidin-4-yl)-4-morpholin-4-yl-1H- | 562.3 | 2.13 | 2 |
| | pyrazolo[3,4-d]pyrimidin-6-yl]-N,N- | | | |
| | dimethylbenzenesulfonamide | | | |
| 70 | 4-[1-(1-benzylpiperidin-4-yl)-4-morpholin-4-yl-1H- | 566.3 | 2.16 | 2 |
| | pyrazolo[3,4-d]pyrimidin-6-yl]-N-cyclopentylbenzamide | | | |
| 71 | 4-[1-(1-benzylpiperidin-4-yl)-4-morpholin-4-yl-1H- | 526.3 | 1.99 | 2 |
| | pyrazolo[3,4-d]pyrimidin-6-yl]-N,N-dimethylbenzamide | | | |
| 72 | 1-(1-benzylpiperidin-4-yl)-6-(1H-indol-7-yl)-4- | 494.26 | | 2 |
| | morpholin-4-yl-1H-pyrazolo[3,4-d]pyrimidine | | | |
| 73 | 1-(1-benzylpiperidin-4-yl)-6-dibenzo[b,d]thien-4-yl-4- | Not | | 2 |
| | morpholin-4-yl-1H-pyrazolo[3,4-d]pyrimidine | Determined | | |
| 74 | 1-(1-benzylpiperidin-4-yl)-6-biphenyl-4-yl-4-morpholin- | Not | | 2 |
| | 4-yl-1H-pyrazolo[3,4-d]pyrimidine | Determined | | |
| 75 | 1-(1-benzylpiperidin-4-yl)-6-biphenyl-3-yl-4-morpholin- | Not | | 2 |
| | 4-yl-1H-pyrazolo[3,4-d]pyrimidine | Determined | | |
| 76 | 3-({4-[6-(1H-indol-5-yl)-4-morpholin-4-yl-1H- | 510.3 | | 3 |
| | pyrazolo[3,4-d]pyrimidin-1-yl]piperidin-1- | | | |
| | yl}methyl)phenol | | | |
| 77 | N-{3-[1-(1-benzylpiperidin-4-yl)-4-morpholin-4-yl-1H- | 498.3 | 1.98 | |
| | pyrazolo[3,4-d]pyrimidin-6-yl]phenyl}formamide | | | |
| 78 | N-{3-[1-(1-benzylpiperidin-4-yl)-4-morpholin-4-yl-1H- | 512.3 | 2 | |
| | pyrazolo[3,4-d]pyrimidin-6-yl]phenyl}acetamide | | | |
| 79 | 4-[1-(1-benzylpiperidin-4-yl)-4-morpholin-4-yl-1H- | 470.3 | 1.9 | 2 |
| | pyrazolo[3,4-d]pyrimidin-6-yl]aniline | | | |
| 80 | N-{4-[1-(1-benzylpiperidin-4-yl)-4-morpholin-4-yl-1H- | 512.3 | 1.98 | 2 |
| | pyrazolo[3,4-d]pyrimidin-6-yl]phenyl}acetamide | | | |
| 81 | 4-({4-[1-(1-benzylpiperidin-4-yl)-4-morpholin-4-yl-1H- | 570.3 | 1.94 | 2 |
| | pyrazolo[3,4-d]pyrimidin-6-yl]phenyl}amino)-4- | | | |
| | oxobutanoic acid | | | |
| 82 | 3-[1-(1-benzylpiperidin-4-yl)-4-morpholin-4-yl-1H- | 498.3 | 1.93 | 2 |
| | pyrazolo[3,4-d]pyrimidin-6-yl]benzamide | | | |
| 83 | 3-[1-(1-benzylpiperidin-4-yl)-4-morpholin-4-yl-1H- | 534.2 | 1.93 | 2 |
| | pyrazolo[3,4-d]pyrimidin-6-yl]benzenesulfonamide | | | |
| 84 | N-{3-[1-(1-benzylpiperidin-4-yl)-4-morpholin-4-yl-1H- | 548.2 | 2 | 2 |
| | pyrazolo[3,4-d]pyrimidin-6- | | | |
| | yl]phenyl}methanesulfonamide | | | |
| 85 | 4-[1-(1-benzylpiperidin-4-yl)-4-morpholin-4-yl-1H- | 534.2 | 1.92 | 2 |
| | pyrazolo[3,4-d]pyrimidin-6-yl]benzenesulfonamide | | | |
| 86 | 4-[1-(1-benzylpiperidin-4-yl)-4-morpholin-4-yl-1H- | 548.2 | 2.01 | 2 |
| | pyrazolo[3,4-d]pyrimidin-6-yl]-N- | | | |
| | methylbenzenesulfonamide | | | |
| 87 | 4-[1-(1-benzylpiperidin-4-yl)-4-morpholin-4-yl-1H- | 562.3 | 2.16 | 2 |
| | pyrazolo[3,4-d]pyrimidin-6-yl]-N,N- | | | |
| | dimethylbenzenesulfonamide | | | |
| 88 | 1-(1-benzylpiperidin-4-yl)-6-(3,5-difluorophenyl)-4- | 491.2 | 2.32 | 3 |
| | morpholin-4-yl-1H-pyrazolo[3,4-d]pyrimidine | | | |
| 89 | 1-(1-benzylpiperidin-4-yl)-6-(1-methyl-1H-indol-5-yl)-4- | 508.3 | 2.25 | 3 |
| | morpholin-4-yl-1H-pyrazolo[3,4-d]pyrimidine | | | |
| 90 | 1-cyclohexyl-6-(1H-indol-5-yl)-4-morpholin-4-yl-1H- | 403.2 | | 2 |
| | pyrazolo[3,4-d]pyrimidine | | | |
| 91 | 3-(1-cyclohexyl-4-morpholin-4-yl-1H-pyrazolo[3,4- | 380.2 | | 2 |
| | d]pyrimidin-6-yl)phenol | | | |
| 92 | 6-(1H-indol-5-yl)-4-morpholin-4-yl-1-[1- | 544.2 | | 3 |
| | (phenylsulfonyl)piperidin-4-yl]-1H-pyrazolo[3,4- | | | |
| | d]pyrimidine | | | |
| 93 | 1-[1-(chloroacetyl)piperidin-4-yl]-6-(1H-indol-5-yl)-4- | 480.2 | | 3 |
| | morpholin-4-yl-1H-pyrazolo[3,4-d]pyrimidine | | | |
| 94 | 2-{4-[6-(1H-indol-5-yl)-4-morpholin-4-yl-1H- | 489.3 | | 7 |
| | pyrazolo[3,4-d]pyrimidin-1-yl]piperidin-1-yl}-N,N- | | | |
| | dimethyl-2-oxoethanamine | | | |
| 95 | 4-[6-(1H-indol-5-yl)-4-morpholin-4-yl-1H-pyrazolo[3,4- | 475.3 | | 7 |
| | d]pyrimidin-1-yl]-N,N-dimethylpiperidine-1- | | | |
| | carboxamide | | | |
| 96 | 2-{4-[6-(1H-indol-5-yl)-4-morpholin-4-yl-1H- | 475.3 | | 3 |
| | pyrazolo[3,4-d]pyrimidin-1-yl]piperidin-1-yl}-N-methyl- | | | |
| | 2-oxoethanamine | | | |
| 97 | 3-(1-ethyl-4-morpholin-4-yl-1H-pyrazolo[3,4- | 326.2 | | 4 |
| | d]pyrimidin-6-yl)phenol | | | |
| 98 | 6-(3,6-dihydro-2H-pyran-4-yl)-4-morpholin-4-yl-1- | 364.3 | | 2 |
| | phenyl-1H-pyrazolo[3,4-d]pyrimidine | | | |
| 99 | N-[4-(4-morpholin-4-yl-1-phenyl-1H-pyrazolo[3,4- | 451.1 | 2.38 | 2 |
| | d]pyrimidin-6-yl)phenyl]methanesulfonamide | | | |
| 100 | 4-(4-morpholin-4-yl-1-phenyl-1H-pyrazolo[3,4- | 401.2 | 2.29 | 2 |
| | d]pyrimidin-6-yl)benzamide | | | |
| 101 | methyl [4-(4-morpholin-4-yl-1-phenyl-1H-pyrazolo[3,4- | 431.2 | 2.51 | 8 |
| | d]pyrimidin-6-yl)phenyl]carbamate | | | |
| 102 | N-[3-(dimethylamino)propyl]-4-(4-morpholin-4-yl-1- | 486.3 | 2.07 | 2 |
| | phenyl-1H-pyrazolo[3,4-d]pyrimidin-6-yl)benzamide | | | |
| 103 | N-[2-(dimethylamino)ethyl]-4-(4-morpholin-4-yl-1- | 472.2 | 2.05 | 2 |
| | phenyl-1H-pyrazolo[3,4-d]pyrimidin-6-yl)benzamide | | | |
| 104 | N-[2-(dimethylamino)ethyl]-3-(4-morpholin-4-yl-1- | 472.2 | 2.05 | 2 |
| | phenyl-1H-pyrazolo[3,4-d]pyrimidin-6-yl)benzamide | | | |
| 105 | 6-(1-benzothien-5-yl)-4-morpholin-4-yl-1-phenyl-1H- | 414.1 | 2.88 | 2 |
| | pyrazolo[3,4-d]pyrimidine | | | |
| 106 | 4-(4-morpholin-4-yl-1-phenyl-1H-pyrazolo[3,4- | 373.2 | 2.33 | 2 |
| | d]pyrimidin-6-yl)aniline4-(4-morpholin-4-yl-1-phenyl- | | | |
| | 1H-pyrazolo[3,4-d]pyrimidin-6-yl)aniline | | | |
| 107 | 3-(4-morpholin-4-yl-1-phenyl-1H-pyrazolo[3,4- | 373.2 | 2.19 | 2 |
| | d]pyrimidin-6-yl)aniline | | | |
| 108 | N-[4-(4-morpholin-4-yl-1-phenyl-1H-pyrazolo[3,4- | 415.2 | 2.41 | 2 |
| | d]pyrimidin-6-yl)phenyl]acetamide | | | |
| 109 | 4-{[4-(4-morpholin-4-yl-1-phenyl-1H-pyrazolo[3,4- | 473.2 | 2.29 | 2 |
| | d]pyrimidin-6-yl)phenyl]amino}-4-oxobutanoic acid | | | |
| 110 | 6-(1H-indol-5-yl)-4-morpholin-4-yl-1-phenyl-1H- | 397.2 | 2.52 | 2 |
| | pyrazolo[3,4-d]pyrimidine | | | |
| 111 | 6-(1H-indol-2-yl)-4-morpholin-4-yl-1-phenyl-1H- | 0 | 0 | 2 |
| | pyrazolo[3,4-d]pyrimidine | | | |
| 112 | 6-(1-benzofuran-2-yl)-4-morpholin-4-yl-1-phenyl-1H- | 398.2 | 2.72 | 2 |
| | pyrazolo[3,4-d]pyrimidine | | | |
| 113 | 6-(1-benzothien-2-yl)-4-morpholin-4-yl-1-phenyl-1H- | 414.1 | 2.94 | 2 |
| | pyrazolo[3,4-d]pyrimidine | | | |
| 114 | N,N-dimethyl-4-(4-morpholin-4-yl-1-phenyl-1H- | 401.2 | 2.72 | 2 |
| | pyrazolo[3,4-d]pyrimidin-6-yl)aniline | | | |
| 115 | N-[3-(4-morpholin-4-yl-1-phenyl-1H-pyrazolo[3,4- | 415.2 | 2.43 | 2 |
| | d]pyrimidin-6-yl)phenyl]acetamide | | | |
| 116 | N-[3-(4-morpholin-4-yl-1-phenyl-1H-pyrazolo[3,4- | 451.1 | 2.38 | 2 |
| | d]pyrimidin-6-yl)phenyl]methanesulfonamide | | | |
| 117 | 6-(1-benzofuran-5-yl)-4-morpholin-4-yl-1-phenyl-1H- | 398.2 | 2.77 | 2 |
| | pyrazolo[3,4-d]pyrimidine | | | |
| 118 | N-[4-(4-morpholin-4-yl-1-phenyl-1H-pyrazolo[3,4- | 401.2 | 2.38 | 2 |
| | d]pyrimidin-6-yl)phenyl]formamide | | | |
| 119 | 4-morpholin-4-yl-6-(4-nitrophenyl)-1-phenyl-1H- | 403.2 | | 2 |
| | pyrazolo[3,4-d]pyrimidine | | | |
| 120 | 6-(1H-indol-5-yl)-1-{1-[(4-methylpiperazin-1- | 530.3 | | 10 |
| | yl)carbonyl]piperidin-4-yl}-4-morpholin-4-yl-1H- | | | |
| | pyrazolo[3,4-d]pyrimidine | | | |
| 121 | 6-(1H-indol-5-yl)-4-morpholin-4-yl-1-[1-(morpholin-4- | 517.3 | | 10 |
| | ylcarbonyl)piperidin-4-yl]-1H-pyrazolo[3,4-d]pyrimidine | | | |
| 122 | 4-[6-(1H-indol-5-yl)-4-morpholin-4-yl-1H-pyrazolo[3,4- | 538.2 | 2.25 | 10 |
| | d]pyrimidin-1-yl]-N-pyridin-3-ylpiperidine-1- | | | |
| | carbothioamide | | | |
| 123 | 6-(1H-indol-5-yl)-4-morpholin-4-yl-1-[1-(pyrrolidin-1- | 501.3 | | 10 |
| | ylcarbonyl)piperidin-4-yl]-1H-pyrazolo[3,4-d]pyrimidine | | | |
| 124 | 6-(1H-indol-5-yl)-1-(1-methylpiperidin-4-yl)-4- | 418.2 | 1.94 | 3 |
| | morpholin-4-yl-1H-pyrazolo[3,4-d]pyrimidine | | | |
| 125 | 6-(1H-indol-5-yl)-4-morpholin-4-yl-1-(2-piperidin-1- | 432.2 | 1.89 | 4, 2 |
| | ylethyl)-1H-pyrazolo[3,4-d]pyrimidine | | | |
| 126 | 3-[4-morpholin-4-yl-1-(2-piperidin-1-ylethyl)-1H- | 409.2 | 1.75 | 4, 2 |
| | pyrazolo[3,4-d]pyrimidin-6-yl]phenol | | | |
| 127 | N-{4-[4-morpholin-4-yl-1-(2-piperidin-1-ylethyl)-1H- | 450.3 | 1.77 | 4, 2 |
| | pyrazolo[3,4-d]pyrimidin-6-yl]phenyl}acetamide | | | |
| 128 | N-methyl-4-(4-morpholin-4-yl-1-phenyl-1H-pyrazolo[3,4- | 387.2 | 2.5 | 6 |
| | d]pyrimidin-6-yl)aniline | | | |
| 129 | 5-(4-morpholin-4-yl-1-phenyl-1H-pyrazolo[3,4- | 374.2 | 1.94 | 2 |
| | d]pyrimidin-6-yl)pyridin-2-amine | | | |
| 130 | 4-morpholin-4-yl-6-[4-(morpholin-4-ylmethyl)phenyl]-1- | 457.2 | 2.04 | 2 |
| | phenyl-1H-pyrazolo[3,4-d]pyrimidine | | | |
| 131 | 4-morpholin-4-yl-6-(6-morpholin-4-ylpyridin-3-yl)-1- | 444.2 | 2.48 | 2 |
| | phenyl-1H-pyrazolo[3,4-d]pyrimidine | | | |
| 132 | 4-[6-(1H-indol-5-yl)-4-morpholin-4-yl-1H-pyrazolo[3,4- | 524.2 | 1.98 | 10 |
| | d]pyrimidin-1-yl]-N-pyridin-3-ylpiperidine-1- | | | |
| | carboxamide | | | |
| 133 | N~3~,N~3~-dimethyl-N-[4-(4-morpholin-4-yl-1-phenyl- | 472.2 | 2.07 | 2 |
| | 1H-pyrazolo[3,4-d]pyrimidin-6-yl)phenyl]-beta- | | | |
| | alaninamide | | | |
| 134 | N-[4-(4-morpholin-4-yl-1-phenyl-1H-pyrazolo[3,4- | 444.2 | 2.05 | 2 |
| | d]pyrimidin-6-yl)phenyl]-beta-alaninamide | | | |
| 135 | N~2~,N~2~-dimethyl-N-[4-(4-morpholin-4-yl-1-phenyl- | 458.2 | 2.04 | 2 |
| | 1H-pyrazolo[3,4-d]pyrimidin-6-yl)phenyl]glycinamide | | | |
| 136 | N-[4-(4-morpholin-4-yl-1-phenyl-1H-pyrazolo[3,4- | 430.2 | 2.07 | 2 |
| | d]pyrimidin-6-yl)phenyl]glycinamide | | | |
| 137 | N-[4-(4-morpholin-4-yl-1-phenyl-1H-pyrazolo[3,4- | 512.3 | 2.16 | 2 |
| | d]pyrimidin-6-yl)phenyl]-3-piperidin-1-ylpropanamide | | | |
| 138 | 3-methoxy-N-[4-(4-morpholin-4-yl-1-phenyl-1H- | 459.2 | 2.42 | 2 |
| | pyrazolo[3,4-d]pyrimidin-6-yl)phenyl]propanamide | | | |
| 139 | 3-hydroxy-N-[4-(4-morpholin-4-yl-1-phenyl-1H- | 445.2 | 2.35 | 2 |
| | pyrazolo[3,4-d]pyrimidin-6-yl)phenyl]propanamide | | | |
| 140 | N-[4-(4-morpholin-4-yl-1-phenyl-1H-pyrazolo[3,4- | 484.2 | 2.08 | 5 |
| | d]pyrimidin-6-yl)phenyl]piperidine-4-carboxamide | | | |
| 141 | 1-methyl-N-[4-(4-morpholin-4-yl-1-phenyl-1H- | 498.3 | 2.2 | 5 |
| | pyrazolo[3,4-d]pyrimidin-6-yl)phenyl]piperidine-4- | | | |
| | carboxamide | | | |
| 142 | 1-(1,4-dioxaspiro[4.5]dec-8-yl)-6-(1H-indol-5-yl)-4- | 461.2 | | 9 |
| | morpholin-4-yl-1H-pyrazolo[3,4-d]pyrimidine | | | |
| 143 | 4-[6-(1H-indol-5-yl)-4-morpholin-4-yl-1H-pyrazolo[3,4- | 417.2 | | 9 |
| | d]pyrimidin-1-yl]cyclohexanone | | | |
| 144 | 1-[3-(4-morpholin-4-yl-1-phenyl-1H-pyrazolo[3,4- | 387.2 | 1.98 | 2 |
| | d]pyrimidin-6-yl)phenyl]methanamine | | | |
| 145 | N-[3-(4-morpholin-4-yl-1-phenyl-1H-pyrazolo[3,4- | 429.2 | 2.42 | 5 |
| | d]pyrimidin-6-yl)benzyl]acetamide | | | |
| 146 | 1-[4-(4-morpholin-4-yl-1-phenyl-1H-pyrazolo[3,4- | 387.2 | 1.98 | 2 |
| | d]pyrimidin-6-yl)phenyl]methanamine | | | |
| 147 | N-[4-(4-morpholin-4-yl-1-phenyl-1H-pyrazolo[3,4- | 429.2 | 2.41 | 5 |
| | d]pyrimidin-6-yl)benzyl]acetamide | | | |
| 148 | N-[5-(4-morpholin-4-yl-1-phenyl-1H-pyrazolo[3,4- | 416.2 | 2.47 | 2 |
| | d]pyrimidin-6-yl)pyridin-2-yl]acetamide | | | |
| 149 | N-methyl-N-[4-(4-morpholin-4-yl-1-phenyl-1H- | 429.2 | 2.61 | 6 |
| | pyrazolo[3,4-d]pyrimidin-6-yl)phenyl]acetamide | | | |
| 150 | 4-[6-(1H-indol-5-yl)-4-morpholin-4-yl-1H-pyrazolo[3,4- | 446.3 | 1.99 | 9 |
| | d]pyrimidin-1-yl]-N,N-dimethylcyclohexanamine | | | |
| 151 | 6-(1H-indol-5-yl)-4-morpholin-4-yl-1-(4-pyrrolidin-1- | 472.3 | 2.05 | 9 |
| | ylcyclohexyl)-1H-pyrazolo[3,4-d]pyrimidine | | | |
| 152 | 4-[6-(1H-indol-5-yl)-4-morpholin-4-yl-1H-pyrazolo[3,4- | 419.2 | 2.21 | 9 |
| | d]pyrimidin-1-yl]cyclohexanol | | | |
| 153 | N-{4-[6-(1H-indol-5-yl)-4-morpholin-4-yl-1H- | 524.3 | 2.26 | 9 |
| | pyrazolo[3,4-d]pyrimidin-1-yl]cyclohexyl}-4- | | | |
| | methoxyaniline | | | |
| 154 | methyl {4-[1-(1-benzylpiperidin-4-yl)-4-morpholin-4-yl- | 528.3 | 2.05 | 8 |
| | 1H-pyrazolo[3,4-d]pyrimidin-6-yl]phenyl}carbamate | | | |
| 155 | 5-[1-(1-benzylpiperidin-4-yl)-4-morpholin-4-yl-1H- | 471.3 | 1.62 | 2 |
| | pyrazolo[3,4-d]pyrimidin-6-yl]pyridin-2-amine | | | |
| 156 | 1-(1-benzylpiperidin-4-yl)-6-(6-chloropyridin-3-yl)-4- | 490.2 | 2.12 | 2 |
| | morpholin-4-yl-1H-pyrazolo[3,4-d]pyrimidine | | | |
| 157 | 1-(1-benzylpiperidin-4-yl)-6-(2-chloropyridin-4-yl)-4- | 490.2 | 2.1 | 2 |
| | morpholin-4-yl-1H-pyrazolo[3,4-d]pyrimidine | | | |
| 158 | N-(4-{4-morpholin-4-yl-1-[1-(pyridin-3- | 513.3 | 1.84 | 2 |
| | ylmethyl)piperidin-4-yl]-1H-pyrazolo[3,4-d]pyrimidin-6- | | | |
| | yl}phenyl)acetamide | | | |
| 159 | N-(4-{4-morpholin-4-yl-1-[1-(pyridin-3- | 527.2 | 2.12 | 2 |
| | ylcarbonyl)piperidin-4-yl]-1H-pyrazolo[3,4-d]pyrimidin- | | | |
| | 6-yl}phenyl)acetamide | | | |
| 160 | 3-[6-(1H-indol-5-yl)-4-morpholin-4-yl-1H-pyrazolo[3,4- | 406.2 | 1.87 | 4, 2 |
| | d]pyrimidin-1-yl]-N,N-dimethylpropan-1-amine | | | |
| 161 | 1-{2-[6-(1H-indol-5-yl)-4-morpholin-4-yl-1H- | 432.2 | 2.14 | 4, 2 |
| | pyrazolo[3,4-d]pyrimidin-1-yl]ethyl}pyrrolidin-2-one | | | |
| 162 | 6-(1H-indol-5-yl)-4-morpholin-4-yl-1-(2-piperazin-1- | Not | | 4, 2 |
| | ylethyl)-1H-pyrazolo[3,4-d]pyrimidine | Determined | | |
| 163 | 3-{1-[3-(dimethylamino)propyl]-4-morpholin-4-yl-1H- | 383.2 | 1.73 | 4, 2 |
| | pyrazolo[3,4-d]pyrimidin-6-yl}phenol | | | |
| 164 | 1-{2-[6-(3-hydroxyphenyl)-4-morpholin-4-yl-1H- | 409.2 | 2 | 4, 2 |
| | pyrazolo[3,4-d]pyrimidin-1-yl]ethyl}pyrrolidin-2-one | | | |
| 165 | 3-[4-morpholin-4-yl-1-(2-morpholin-4-ylethyl)-1H- | 411.2 | 1.71 | 4, 2 |
| | pyrazolo[3,4-d]pyrimidin-6-yl]phenol | | | |
| 166 | 3-[4-morpholin-4-yl-1-(2-piperazin-1-ylethyl)-1H- | 410.2 | 1.75 | 4, 2 |
| | pyrazolo[3,4-d]pyrimidin-6-yl]phenol | | | |
| 167 | tert-butyl (3R)-3-[6-(1H-indol-5-yl)-4-morpholin-4-yl- | 504.3 | 2.55 | 4, 2 |
| | 1H-pyrazolo[3,4-d]pyrimidin-1-yl]piperidine-1- | | | |
| | carboxylate | | | |
| 168 | 6-(1H-indol-5-yl)-4-morpholin-4-yl-1-[(3R)-piperidin-3- | 404.2 | 1.91 | 4, 2 |
| | yl]-1H-pyrazolo[3,4-d]pyrimidine | | | |
| 169 | tert-butyl (3S)-3-[6-(1H-indol-5-yl)-4-morpholin-4-yl-1H- | 504.3 | 2.55 | 4, 2 |
| | pyrazolo[3,4-d]pyrimidin-1-yl]piperidine-1-carboxylate | | | |
| 170 | 6-(1H-indol-5-yl)-4-morpholin-4-yl-1-[(3S)-piperidin-3- | ND | ND | 4, 2 |
| | yl]-1H-pyrazolo[3,4-d]pyrimidine | | | |
| 171 | 6-(1H-indol-5-yl)-4-morpholin-4-yl-1-(tetrahydro-2H- | 421.2 | 2.41 | 4, 2 |
| | thiopyran-4-yl)-1H-pyrazolo[3,4-d]pyrimidine | | | |
| 172 | 1-{1-[(6-fluoropyridin-3-yl)methyl]piperidin-4-yl}-6-(1H- | 513.2 | 1.97 | 3 |
| | indol-5-yl)-4-morpholin-4-yl-1H-pyrazolo[3,4- | | | |
| | d]pyrimidine | | | |
| 173 | 1-{1-[(6-bromopyridin-3-yl)methyl]piperidin-4-yl}-6- | 573.2 | 2.07 | 3 |
| | (1H-indol-5-yl)-4-morpholin-4-yl-1H-pyrazolo[3,4- | | | |
| | d]pyrimidine | | | |
| 174 | 1-{1-[(6-chloropyridin-3-yl)methyl]piperidin-4-yl}-6- | 529.2 | 2 | 3 |
| | (1H-indol-5-yl)-4-orpholin-4-yl-1H-pyrazolo[3,4- | | | |
| | d]pyrimidine | | | |
| 175 | 1-{1-[(2-chloropyridin-3-yl)methyl]piperidin-4-yl}-6- | 529.2 | 1.99 | 3 |
| | (1H-indol-5-yl)-4-morpholin-4-yl-1H-pyrazolo[3,4- | | | |
| | d]pyrimidine | | | |
| 176 | 6-(1H-indol-5-yl)-1-{1-[(6-methoxypyridin-3- | 525.3 | 2.01 | 3 |
| | yl)methyl]piperidin-4-yl}-4-morpholin-4-yl-1H- | | | |
| | pyrazolo[3,4-d]pyrimidine | | | |
| 177 | 6-(1H-indol-5-yl)-1-{1-[(2-methoxypyridin-3- | 525.3 | 2.06 | 3 |
| | yl)methyl]piperidin-4-yl}-4-morpholin-4-yl-1H- | | | |
| | pyrazolo[3,4-d]pyrimidine | | | |
| 178 | 6-(1H-indol-5-yl)-4-morpholin-4-yl-1-[1-(pyridin-2- | 495.3 | 1.98 | 3 |
| | ylmethyl)piperidin-4-yl]-1H-pyrazolo[3,4-d]pyrimidine | | | |
| 179 | 6-(1H-indol-5-yl)-4-morpholin-4-yl-1-[1-(pyridin-4- | 495.3 | 1.93 | 3 |
| | ylmethyl)piperidin-4-yl]-1H-pyrazolo[3,4-d]pyrimidine | | | |
| 180 | N-[4-(1-benzyl-4-morpholin-4-yl-1H-pyrazolo[3,4- | 429.3 | | 2 |
| | d]pyrimidin-6-yl)phenyl]acetamide | | | |
| 181 | 4-[1-(1-benzylpiperidin-4-yl)-4-morpholin-4-yl-1H- | 500.3 | 1.93 | 2 |
| | pyrazolo[3,4-d]pyrimidin-6-yl]-2-methoxyaniline | | | |
| 182 | N-{4-[1-(1-benzylpiperidin-4-yl)-4-morpholin-4-yl-1H- | 542.3 | 2.00 | 2 |
| | pyrazolo[3,4-d]pyrimidin-6-yl]-2- | | | |
| | methoxyphenyl}acetamide | | | |
| 183 | 4-[1-(1-benzylpiperidin-4-yl)-4-morpholin-4-yl-1H- | 515.2 | 2.08 | 2 |
| | pyrazolo[3,4-d]pyrimidin-6-yl]-2-nitroaniline | | | |
| 184 | N-{4-[1-(1-benzylpiperidin-4-yl)-4-morpholin-4-yl-1H- | 557.3 | 2.12 | 5 |
| | pyrazolo[3,4-d]pyrimidin-6-yl]-2-nitrophenyl}acetamide | | | |
| 185 | N-{4-[1-(1-benzylpiperidin-4-yl)-4-morpholin-4-yl-1H- | 569.3 | 2.17 | 8 |
| | pyrazolo[3,4-d]pyrimidin-6-yl]phenyl}-N′-butylurea | | | |
| 186 | N-{4-[1-(1-benzylpiperidin-4-yl)-4-morpholin-4-yl-1H- | 540.3 | 2.08 | 5 |
| | pyrazolo[3,4-d]pyrimidin-6-yl]phenyl}-2- | | | |
| | methylpropanamide | | | |
| 187 | N-{4-[1-(1-benzylpiperidin-4-yl)-4-morpholin-4-yl-1H- | 554.3 | 2.15 | 5 |
| | pyrazolo[3,4-d]pyrimidin-6-yl]phenyl}-2,2- | | | |
| | dimethylpropanamide | | | |
| 188 | 1-(1-benzylpiperidin-4-yl)-4-morpholin-4-yl-6-(1H- | 445.2 | 1.80 | 2 |
| | pyrazol-5-yl)-1H-pyrazolo[3,4-d]pyrimidine | | | |
| 189 | 1-(1-benzylpiperidin-4-yl)-4-morpholin-4-yl-6-(1H- | 445.2 | 1.76 | 2 |
| | pyrazol-4-yl)-1H-pyrazolo[3,4-d]pyrimidine | | | |
| 190 | 6-(2,1,3 -benzoxadiazol-5-yl)-1-(1-benzylpiperidin-4-yl)- | 497.2 | 2.19 | 2 |
| | 4-morpholin-4-yl-1H-pyrazolo[3,4-d]pyrimidine | | | |
| 191 | 5-[1-(1-benzylpiperidin-4-yl)-4-morpholin-4-yl-1H- | 472.2 | 1.82 | 2 |
| | pyrazolo[3,4-d]pyrimidin-6-yl]pyrimidin-2-amine | | | |
| 192 | 5-[1-(1-benzylpiperidin-4-yl)-4-morpholin-4-yl-1H- | 472.2 | 1.79 | 2 |
| | pyrazolo[3,4-d]pyrimidin-6-yl]pyridin-2-ol | | | |
| 193 | 5-[1-(1-benzylpiperidin-4-yl)-4-morpholin-4-yl-1H- | 584.3 | 1.66 | 2 |
| | pyrazolo[3,4-d]pyrimidin-6-yl]-N-(2-morpholin-4- | | | |
| | ylethyl)pyridin-2-amine | | | |
| 194 | 1-(1-acryloylpiperidin-4-yl)-6-(1H-indol-5-yl)-4- | 458.5 | 2.21 | 3 |
| | morpholin-4-yl-1H-pyrazolo[3,4-d]pyrimidine | | | |
| 195 | 1-{1-[(2-chloropyridin-3-yl)carbonyl]piperidin-4-yl}-6- | 543.3 | 2.28 | 3 |
| | (1H-indol-5-yl)-4-morpholin-4-yl-1H-pyrazolo[3,4- | | | |
| | d]pyrimidine | | | |
| 196 | 1-{1-[(6-chloropyridin-3-yl)carbonyl]piperidin-4-yl}-6- | 544.0 | 2.31 | 3 |
| | (1H-indol-5-yl)-4-morpholin-4-yl-1H-pyrazolo[3,4- | | | |
| | d]pyrimidine | | | |
| 197 | 6-(1H-indol-5-yl)-4-morpholin-4-yl-1-{1-[(1- | 525.3 | 2.13 | 3 |
| | oxidopyridin-3-yl)carbonyl]piperidin-4-yl}-1H- | | | |
| | pyrazolo[3,4-d]pyrimidine | | | |
| 198 | 2-({4-[6-(1H-indol-5-yl)-4-morpholin-4-yl-1H- | 523.6 | 2.14 | 3 |
| | pyrazolo[3,4-d]pyrimidin-1-yl]piperidin-1- | | | |
| | yl}carbonyl)pyridin-3-ol | | | |
| 199 | 6-(1H-indol-5-yl)-1-{1-[(4-methylpyridin-3- | 523.2 | 2.18 | 3 |
| | yl)carbonyl]piperidin-4-yl}-4-morpholin-4-yl-1H- | | | |
| | pyrazolo[3,4-d]pyrimidine | | | |
| 200 | 6-(1H-indol-5-yl)-1-{1-[(2-methylpyridin-3- | 523.2 | 2.17 | 3 |
| | yl)carbonyl]piperidin-4-yl}-4-morpholin-4-yl-1H- | | | |
| | pyrazolo[3,4-d]pyrimidine | | | |
| 201 | 1-{1-[(6-fluoropyridin-3-yl)carbonyl]piperidin-4-yl}-6- | 527.6 | 2.25 | 3 |
| | (1H-indol-5-yl)-4-morpholin-4-yl-1H-pyrazolo[3,4- | | | |
| | d]pyrimidine | | | |
| 202 | 6-(1H-indol-5-yl)-1-{1-[(6-methylpyridin-3- | 523.6 | 2.17 | 3 |
| | yl)carbonyl]piperidin-4-yl}-4-morpholin-4-yl-1H- | | | |
| | pyrazolo[3,4-d]pyrimidine | | | |
| 203 | 1-{1-[(6-bromopyridin-3-yl)carbonyl]piperidin-4-yl}-6- | 587.1 | 2.35 | 3 |
| | (1H-indol-5-yl)-4-morpholin-4-yl-1H-pyrazolo[3,4- | | | |
| | d]pyrimidine | | | |
| 204 | 3-(4-morpholin-4-yl-1H-pyrazolo[3,4-d]pyrimidin-6- | 298.1 | 1.71 | 11 |
| | yl)phenol | | | |
| 205 | N′-{4-[1-(1-benzylpiperidin-4-yl)-4-morpholin-4-yl-1H- | 541.3 | 1.98 | 8 |
| | pyrazolo[3,4-d]pyrimidin-6-yl]phenyl}-N,N-dimethylurea | | | |
| 206 | N-{4-[1-(1-benzylpiperidin-4-yl)-4-morpholin-4-yl-1H- | 527.6 | 1.90 | 8 |
| | pyrazolo[3,4-d]pyrimidin-6-yl]phenyl}-N′-methylurea | | | |
| 207 | ethyl {4-[1-(1-benzylpiperidin-4-yl)-4-morpholin-4-yl- | 542.3 | 2.12 | 8 |
| | 1H-pyrazolo[3,4-d]pyrimidin-6-yl]phenyl}carbamate | | | |
| 208 | isopropyl{4-[1-(1-benzylpiperidin-4-yl)-4-morpholin-4- | 556.3 | 2.19 | 8 |
| | yl-1H-pyrazolo[3,4-d]pyrimidin-6-yl]phenyl}carbamate | | | |
| 209 | methyl (4-{4-morpholin-4-yl-1-[1-(pyridin-3- | 529.3 | 1.91 | 8 |
| | ylmethyl)piperidin-4-yl]-1H-pyrazolo[3,4-d]pyrimidin-6- | | | |
| | yl}phenyl)carbamate | | | |
| 210 | 5-{4-morpholin-4-yl-1-[1-(pyridin-3-ylmethyl)piperidin- | 472.3 | 1.52 | 2 |
| | 4-yl]-1H-pyrazolo[3,4-d]pyrimidin-6-yl}pyridin-2-amine | | | |
| 211 | 5-{4-morpholin-4-yl-1-[1-(pyridin-3-ylmethyl)piperidin- | 473.4 | 1.68 | 2 |
| | 4-yl]-1H-pyrazolo[3,4-d]pyrimidin-6-yl}pyrimidin-2- | | | |
| | amine | | | |
| 212 | 1-butyl-3-(4-{4-morpholin-4-yl-1-[1-(pyridin-3- | 682.7 | 2.01 | 8 |
| | ylmethyl)piperidin-4-yl]-1H-pyrazolo[3,4-d]pyrimidin-6- | | | |
| | yl}phenyl)urea | | | |
| 213 | methyl (4-{4-morpholin-4-yl-1-[1-(pyridin-3- | 541.6 | 2.21 | 8 |
| | ylcarbonyl)piperidin-4-yl]-1H-pyrazolo[3,4-d]pyrimidin- | | | |
| | 6-yl}phenyl)carbamate | | | |
| 214 | 5-{4-morpholin-4-yl-1-[1-(pyridin-3- | 486.6 | 1.74 | 2 |
| | ylcarbonyl)piperidin-4-yl]-1H-pyrazolo[3,4-d]pyrimidin- | | | |
| | 6-yl}pyridin-2-amine | | | |
| 215 | 5-{4-morpholin-4-yl-1-[1-(pyridin-3- | 487.5 | 1.92 | 2 |
| | ylcarbonyl)piperidin-4-yl]-1H-pyrazolo[3,4-d]pyrimidin- | | | |
| | 6-yl}pyrimidin-2-amine | | | |
| 216 | 1-butyl-3-(4-{4-morpholin-4-yl-1-[1-(pyridin-3- | 582.7 | 2.26 | 8 |
| | ylcarbonyl)piperidin-4-yl]-1H-pyrazolo[3,4-d]pyrimidin- | | | |
| | 6-yl}phenyl)urea | | | |
| 217 | 6-(1H-indol-5-yl)-4-morpholin-4-yl-1-(1-pyridin-2- | 481.25 | | 12 |
| | ylpiperidin-4-yl)-1H-pyrazolo[3,4-d]pyrimidine | | | |
| 218 | N-methyl-N′-(4-{4-morpholin-4-yl-1-[1-(pyridin-3- | 528.3 | 1.77 | 13, 3 |
| | ylmethyl)piperidin-4-yl]-1H-pyrazolo[3,4-d]pyrimidin-6- | | | |
| | yl}phenyl)urea | | | |
| 219 | N-methyl-N′-(4-{4-morpholin-4-yl-1-[1-(pyridin-3- | 542.3 | 2.01 | 13, 3 |
| | ylcarbonyl)piperidin-4-yl]-1H-pyrazolo[3,4-d]pyrimidin- | | | |
| | 6-yl}phenyl)urea | | | |
| 220 | N-(4-{4-morpholin-4-yl-1-[1-(pyridin-3- | 513.3 | 1.84 | 3 |
| | ylmethyl)piperidin-4-yl]-1H-pyrazolo[3,4-d]pyrimidin-6- | | | |
| | yl}phenyl)acetamide | | | |
| 221 | N-(4-{4-morpholin-4-yl-1-[1-(pyridin-3- | 527.2 | 2.12 | 3 |
| | ylcarbonyl)piperidin-4-yl]-1H-pyrazolo[3,4-d]pyrimidin- | | | |
| | 6-yl}phenyl)acetamide | | | |
| 222 | methyl (4-{4-morpholin-4-yl-1-[1-(pyridin-3- | 529.3 | 1.91 | 3 |
| | ylmethyl)piperidin-4-yl]-1H-pyrazolo[3,4-d]pyrimidin-6- | | | |
| | yl}phenyl)carbamate | | | |
| 223 | methyl (4-{4-morpholin-4-yl-1-[1-(pyridin-3- | 541.6 | 2.2 | 3 |
| | ylcarbonyl)piperidin-4-yl]-1H-pyrazolo[3,4-d]pyrimidin- | | | |
| | 6-yl}phenyl)carbamate | | | |
| 224 | N-{4-[4-morpholin-4-yl-1-(tetrahydro-2H-pyran-2-yl)- | 423.21398¹ | | 21 |
| | 1H-pyrazolo[3,4-d]pyrimidin-6-yl]phenyl}acetamide | | | |
| 225 | 1-methyl-3-(4-{4-morpholin-4-yl-1-[1-(pyridin-3- | 528.3 | 1.77 | 13, then |
| | ylmethyl)piperidin-4-yl]-1H-pyrazolo[3,4-d]pyrimidin-6- | | | 3, 14 or |
| | yl}phenyl)urea | | | 15 |
| 226 | 1-methyl-3-(4-{4-morpholin-4-yl-1-[1-(pyridin-3- | 542.3 | 2.01 | 13, then |
| | ylcarbonyl)piperidin-4-yl]-1H-pyrazolo[3,4-d]pyrimidin- | | | 3 or 14 |
| | 6-yl}phenyl)urea | | | |
| 227 | methyl {3-[1-(1-benzylpiperidin-4-yl)-4-morpholin-4-yl- | 528.3 | 2.03 | 3 |
| | 1H-pyrazolo[3,4-d]pyrimidin-6-yl]phenyl}carbamate | | | |
| 228 | N-{3-[1-(1-benzylpiperidin-4-yl)-4-morpholin-4-yl-1H- | 527.3 | 1.91 | 13, then 3 |
| | pyrazolo[3,4-d]pyrimidin-6-yl]phenyl}-N′-methylurea | | | |
| 229 | N-{3-[1-(1-benzylpiperidin-4-yl)-4-morpholin-4-yl-1H- | 513.3 | 1.91 | 13, then 3 |
| | pyrazolo[3,4-d]pyrimidin-6-yl]phenyl}urea | | | |
| 230 | 3-[1-(1-benzylpiperidin-4-yl)-4-morpholin-4-yl-1H- | 506.3 | 2.04 | 3 |
| | pyrazolo[3,4-d]pyrimidin-6-yl]quinoline | | | |
| 231 | N-{4-[1-(1-benzylpiperidin-4-yl)-4-morpholin-4-yl-1H- | 498.3 | 1.89 | 3 |
| | pyrazolo[3,4-d]pyrimidin-6-yl]phenyl}formamide | | | |
| 232 | 4-[1-(1-benzylpiperidin-4-yl)-4-morpholin-4-yl-1H- | 471.2 | 1.9 | 3 |
| | pyrazolo[3,4-d]pyrimidin-6-yl]phenol | | | |
| 233 | N-[4-(4-morpholin-4-yl-1H-pyrazolo[3,4-d]pyrimidin-6- | 339.15708¹ | | 21 |
| | yl)phenyl]acetamide | | | |
| 234 | 4-{4-[6-(1H-indol-5-yl)-4-morpholin-4-yl-1H- | Not | | 7 |
| | pyrazolo[3,4-d]pyrimidin-1-yl]piperidin-1-yl}-N,N- | Determined | | |
| | dimethyl-4-oxobut-2-en-1-amine | | | |
| 235 | 1-methyl-3-{4-[4-morpholin-4-yl-1-(tetrahydro-2H-pyran- | 438.22554¹ | | 21 |
| | 2-yl)-1H-pyrazolo[3,4-d]pyrimidin-6-yl]phenyl}urea | | | |
| 236 | 1-methyl-3-[4-(4-morpholin-4-yl-1H-pyrazolo[3,4- | 354.168¹ | | 14, 21 |
| | d]pyrimidin-6-yl)phenyl]urea | | | |

¹High Resolution Mass Spectral Analysis

| | | | | |
|---|---|---|---|---|
| 237 | 6-(1H-indol-5-yl)-4-morpholin-4-yl-1-(tetrahydro-2H- | 405.20491¹ | | 21 |
| | pyran-2-yl)-1H-pyrazolo[3,4-d]pyrimidine | | | |
| 238 | 6-(1H-indol-5-yl)-1-(1-isopropylpiperidin-4-yl)-4- | 446.3 | 1.89 | 8 |
| | morpholin-4-yl-1H-pyrazolo[3,4-d]pyrimidine | | | |
| 239 | 6-(1H-indol-5-yl)-4-morpholin-4-yl-1-[1-(2- | 508.3 | 2.01 | 23 |
| | phenylethyl)piperidin-4-yl]-1H-pyrazolo[3,4-d]pyrimidine | | | |
| 240 | 6-(1H-indol-5-yl)-4-morpholin-4-yl-1-[1-(1- | 508.3 | 2.03 | 23 |
| | phenylethyl)piperidin-4-yl]-1H-pyrazolo[3,4-d]pyrimidine | | | |
| 241 | 6-(1H-indol-5-yl)-1-[1-(2-methoxyethyl)piperidin-4-yl]-4- | 462.3 | 1.87 | 23 |
| | morpholin-4-yl-1H-pyrazolo[3,4-d]pyrimidine | | | |
| 242 | 6-(1H-indol-5-yl)-4-morpholin-4-yl-1-[1- | 522.3 | 2.31 | 7 |
| | (phenylacetyl)piperidin-4-yl]-1H-pyrazolo[3,4- | | | |
| | d]pyrimidine | | | |
| 243 | phenyl 4-[6-(1H-indol-5-yl)-4-morpholin-4-yl-1H- | 524.2 | 2.42 | 7 |
| | pyrazolo[3,4-d]pyrimidin-1-yl]piperidine-1-carboxylate | | | |
| 244 | methyl 4-[6-(1H-indol-5-yl)-4-morpholin-4-yl-1H- | 462.2 | 2.23 | 7 |
| | pyrazolo[3,4-d]pyrimidin-1-yl]piperidine-1-carboxylate | | | |
| 245 | 2-{4-[6-(1H-indol-5-yl)-4-morpholin-4-yl-1H- | 461.2 | 1.78 | 23 |
| | pyrazolo[3,4-d]pyrimidin-1-yl]piperidin-1-yl}acetamide | | | |
| 246 | 2-{4-[6-(1H-indol-5-yl)-4-morpholin-4-yl-1H- | 448.2 | 1.8 | 23 |
| | pyrazolo[3,4-d]pyrimidin-1-yl]piperidin-1-yl}ethanol | | | |
| 247 | 3-{4-[6-(1H-indol-5-yl)-4-morpholin-4-yl-1H- | 462.3 | 1.81 | 23 |
| | pyrazolo[3,4-d]pyrimidin-1-yl]piperidin-1-yl}propan-1-ol | | | |
| 248 | 6-(1H-indol-5-yl)-4-morpholin-4-yl-1H-pyrazolo[3,4- | 321.3 | | 21 |
| | d]pyrimidine | | | |
| 249 | 1-(1-benzylpiperidin-4-yl)-6-[5- | 516.5 | | 3 |
| | (methoxymethoxy)pyridin-3-yl]-4-morpholin-4-yl-1H- | | | |
| | pyrazolo[3,4-d]pyrimidine | | | |
| 250 | 5-[1-(1-benzylpiperidin-4-yl)-4-morpholin-4-yl-1H- | 472.4 | | 3, then |
| | pyrazolo[3,4-d]pyrimidin-6-yl]pyridin-3-ol | | | HCl/Me |
| | | | | OH |
| 251 | 4-[6-(1H-indol-5-yl)-4-morpholin-4-yl-1H-pyrazolo[3,4- | 432.2 | 2.07 | 8 |
| | d]pyrimidin-1-yl]piperidine-1-carbaldehyde | | | |
| 252 | 1-{4-[1-(1-benzylpiperidin-4-yl)-4-morpholin-4-yl-1H- | 513.3 | 1.81 | 13, then 3 |
| | pyrazolo[3,4-d]pyrimidin-6-yl]phenyl}urea | | | |
| 253 | 1-{4-[1-(1-benzylpiperidin-4-yl)-4-morpholin-4-yl-1H- | 541.3 | 1.91 | 13, then 3 |
| | pyrazolo[3,4-d]pyrimidin-6-yl]phenyl}-3-ethylurea | | | |
| 254 | 1-{4-[1-(1-benzylpiperidin-4-yl)-4-morpholin-4-yl-1H- | 555.3 | 1.97 | 13, then 3 |
| | pyrazolo[3,4-d]pyrimidin-6-yl]phenyl}-3-propylurea | | | |
| 255 | propyl {4-[1-(1-benzylpiperidin-4-yl)-4-morpholin-4-yl- | 556.3 | 2.13 | 13, then 3 |
| | 1H-pyrazolo[3,4-d]pyrimidin-6-yl]phenyl}carbamate | | | |
| 256 | 1-{4-[1-(1-benzylpiperidin-4-yl)-4-morpholin-4-yl-1H- | 555.3 | 1.97 | 13, then 3 |
| | pyrazolo[3,4-d]pyrimidin-6-yl]phenyl}-3-isopropylurea | | | |
| 257 | 1-{4-[1-(1-benzylpiperidin-4-yl)-4-morpholin-4-yl-1H- | 589.3 | 2.1 | 13, then 3 |
| | pyrazolo[3,4-d]pyrimidin-6-yl]phenyl}-3-phenylurea | | | |
| 258 | 1-benzyl-3-{4-[1-(1-benzylpiperidin-4-yl)-4-morpholin-4- | 603.3 | 2.06 | 13, then 3 |
| | yl-1H-pyrazolo[3,4-d]pyrimidin-6-yl]phenyl}urea | | | |
| 259 | 1-{4-[1-(1-benzylpiperidin-4-yl)-4-morpholin-4-yl-1H- | 617.3 | 2.11 | 13, then 3 |
| | pyrazolo[3,4-d]pyrimidin-6-yl]phenyl}-3-(2- | | | |
| | phenylethyl)urea | | | |
| 260 | 1-{4-[1-(1-benzylpiperidin-4-yl)-4-morpholin-4-yl-1H- | 631.3 | 2.16 | 13, then 3 |
| | pyrazolo[3,4-d]pyrimidin-6-yl]phenyl}-3-(3- | | | |
| | phenylpropyl)urea | | | |
| 261 | 1-{4-[1-(1-benzylpiperidin-4-yl)-4-morpholin-4-yl-1H- | 590.3 | 1.81 | 13, then 3 |
| | pyrazolo[3,4-d]pyrimidin-6-yl]phenyl}-3-pyridin-3- | | | |
| | ylurea | | | |
| 262 | 1-{4-[1-(1-benzylpiperidin-4-yl)-4-morpholin-4-yl-1H- | 567.3 | 1.99 | 13, then 3 |
| | pyrazolo[3,4-d]pyrimidin-6-yl]phenyl}-3- | | | |
| | (cyclopropylmethyl)urea | | | |
| 263 | 1-allyl-3-{4-[1-(1-benzylpiperidin-4-yl)-4-morpholin-4- | 553.3 | 1.95 | 13, then 3 |
| | yl-1H-pyrazolo[3,4-d]pyrimidin-6-yl]phenyl}urea | | | |
| 264 | 4-[4-morpholin-4-yl-1-(tetrahydro-2H-pyran-2-yl)-1H- | 381.20514¹ | | 21 |
| | pyrazolo[3,4-d]pyrimidin-6-yl]aniline | | | |
| 265 | 1-{4-[1-(1-benzylpiperidin-4-yl)-4-morpholin-4-yl-1H- | 557.3 | 1.79 | 13, then 3 |
| | pyrazolo[3,4-d]pyrimidin-6-yl]phenyl}-3-(2- | | | |
| | hydroxyethyl)urea | | | |
| 266 | 1-{4-[1-(1-benzylpiperidin-4-yl)-4-morpholin-4-yl-1H- | 571.3 | 1.88 | 13, then 3 |
| | pyrazolo[3,4-d]pyrimidin-6-yl]phenyl}-3-(2- | | | |
| | methoxyethyl)urea | | | |
| 267 | 1-(2-aminoethyl)-3-{4-[1-(1-benzylpiperidin-4-yl)-4- | 556.3 | 1.62 | 13, then 3 |
| | morpholin-4-yl-1H-pyrazolo[3,4-d]pyrimidin-6- | | | |
| | yl]phenyl}urea | | | |
| 268 | 1-{4-[1-(1-benzylpiperidin-4-yl)-4-morpholin-4-yl-1H- | 584.3 | 1.65 | 13, then 3 |
| | pyrazolo[3,4-d]pyrimidin-6-yl]phenyl}-3-[2- | | | |
| | (dimethylamino)ethyl]urea | | | |
| 269 | 1-{4-[1-(1-benzylpiperidin-4-yl)-4-morpholin-4-yl-1H- | 571.3 | 1.81 | 13, then 3 |
| | pyrazolo[3,4-d]pyrimidin-6-yl]phenyl}-3-(3- | | | |
| | hydroxypropyl)urea | | | |
| 270 | 1-{4-[1-(1-benzylpiperidin-4-yl)-4-morpholin-4-yl-1H- | 585.3 | 1.9 | 13, then 3 |
| | pyrazolo[3,4-d]pyrimidin-6-yl]phenyl}-3-(3- | | | |
| | methoxypropyl)urea | | | |
| 271 | 1-{4-[1-(1-benzylpiperidin-4-yl)-4-morpholin-4-yl-1H- | 598.4 | 1.67 | 13, then 3 |
| | pyrazolo[3,4-d]pyrimidin-6-yl]phenyl}-3-[3- | | | |
| | (dimethylamino)propyl]urea | | | |
| 272 | 1-{4-[1-(1-benzylpiperidin-4-yl)-4-morpholin-4-yl-1H | 610.4 | 1.68 | 13, then 3 |
| | pyrazolo[3,4-d]pyrimidin-6-yl]phenyl}-3-(1- | | | |
| | methylpiperidin-4-yl)urea | | | |
| 273 | 3-methoxy-N-{4-[4-morpholin-4-yl-1-(tetrahydro-2H- | 515.2 | 2.39 | 14 |
| | pyran-2-yl)-1H-pyrazolo[3,4-d]pyrimidin-6- | | | |
| | yl]phenyl}benzamide | | | |
| 274 | methyl {4-[4-morpholin-4-yl-1-(tetrahydro-2H-pyran-2- | 439.2 | 2.2 | 14 |
| | yl)-1H-pyrazolo[3,4-d]pyrimidin-6-yl]phenyl}carbamate | | | |
| 275 | N~2~,N~2~-dimethyl-N-{4-[4-morpholin-4-yl-1- | 466.2 | 1.8 | 14 |
| | (tetrahydro-2H-pyran-2-yl)-1H-pyrazolo[3,4- | | | |
| | d]pyrimidin-6-yl]phenyl}glycinamide | | | |
| 276 | 2-[4-(dimethylamino)phenyl]-N-{4-[4-morpholin-4-yl-1- | 542.3 | 2.02 | 14 |
| | (tetrahydro-2H-pyran-2-yl)-1H-pyrazolo[3,4- | | | |
| | d]pyrimidin-6-yl]phenyl}acetamide | | | |
| 277 | 3-methoxy-N-[4-(4-morpholin-4-yl-1H-pyrazolo[3,4- | 431.2 | 2.05 | 14 |
| | d]pyrimidin-6-yl)phenyl]benzamide | | | |
| 278 | N-[4-(4-morpholin-4-yl-1H-pyrazolo[3,4-d]pyrimidin-6- | 402.2 | 1.82 | 14 |
| | yl)phenyl]nicotinamide | | | |
| 279 | methyl [4-(4-morpholin-4-yl-1H-pyrazolo[3,4- | 355.1 | 1.78 | 14 |
| | d]pyrimidin-6-yl)phenyl]carbamate | | | |
| 280 | N-[4-(4-morpholin-4-yl-1H-pyrazolo[3,4-d]pyrimidin-6- | 351.1 | 1.78 | 14 |
| | yl)phenyl]acrylamide | | | |
| 281 | N~2~,N~2~-dimethyl-N-[4-(4-morpholin-4-yl-1H- | Not | | 14 |
| | pyrazolo[3,4-d]pyrimidin-6-yl)phenyl]glycinamide | Determined | | |
| 282 | N-[4-(4-morpholin-4-yl-1H-pyrazolo[3,4-d]pyrimidin-6- | 354.2 | 1.46 | 14 |
| | yl)phenyl]glycinamide | | | |
| 283 | N-[4-(4-morpholin-4-yl-1H-pyrazolo[3,4-d]pyrimidin-6- | 368.2 | 1.49 | 21 |
| | yl)phenyl]-beta-alaninamide | | | |
| 284 | 1-methyl-N-[4-(4-morpholin-4-yl-1H-pyrazolo[3,4- | 422.2 | 1.59 | 21 |
| | d]pyrimidin-6-yl)phenyl]piperidine-4-carboxamide | | | |
| 285 | 4-(4-morpholin-4-yl-1H-pyrazolo[3,4-d]pyrimidin-6- | 297.1 | 1.56 | 21 |
| | yl)aniline | | | |
| 286 | 1-{4-[1-(1-benzylpiperidin-4-yl)-4-morpholin-4-yl-1H- | 543.3 | 1.94 | 13, then 3 |
| | pyrazolo[3,4-d]pyrimidin-6-yl]phenyl}-3-methoxyurea | | | |
| 287 | 1-{4-[1-(1-benzylpiperidin-4-yl)-4-morpholin-4-yl-1H- | 557.3 | 2 | 13, then 3 |
| | pyrazolo[3,4-d]pyrimidin-6-yl]phenyl}-3-ethoxyurea | | | |
| 288 | 1-{4-[1-(1-benzylpiperidin-4-yl)-4-morpholin-4-yl-1H- | 559.3 | 1.94 | 13, then 3 |
| | pyrazolo[3,4-d]pyrimidin-6-yl]phenyl}-3-(2- | | | |
| | fluoroethyl)urea | | | |
| 289 | 1-{4-[1-(1-benzylpiperidin-4-yl)-4-morpholin-4-yl-1H- | 595.3 | 2.02 | 13, then 3 |
| | pyrazolo[3,4-d]pyrimidin-6-yl]phenyl}-3-(2,2,2- | | | |
| | trifluoroethyl)urea | | | |
| 290 | N-{4-[1-(1-benzylpiperidin-4-yl)-4-morpholin-4-yl-1H- | 556.3 | 1.99 | 13, then 3 |
| | pyrazolo[3,4-d]pyrimidin-6-yl]phenyl}-2,2- | | | |
| | dimethylhydrazinecarboxamide | | | |
| 291 | 1-{4-[1-(1-benzylpiperidin-4-yl)-4-morpholin-4-yl-1H- | 582.3 | 2.04 | 13, then 3 |
| | pyrazolo[3,4-d]pyrimidin-6-yl]phenyl}-3-pyrrolidin-1- | | | |
| | ylurea | | | |
| 292 | N-[4-(4-morpholin-4-yl-1-phenyl-1H-pyrazolo[3,4- | 431.2 | 2.18 | 14 |
| | d]pyrimidin-6-yl)phenyl]hydrazinecarboxamide | | | |
| 293 | 1-hydroxy-3-[4-(4-morpholin-4-yl-1-phenyl-1H- | 432.2 | 2.19 | 14 |
| | pyrazolo[3,4-d]pyrimidin-6-yl)phenyl]urea | | | |
| 294 | 1-(2-fluoroethyl)-3-[4-(4-morpholin-4-yl-1-phenyl-1H- | 462.2 | 2.31 | 14 |
| | pyrazolo[3,4-d]pyrimidin-6-yl)phenyl]urea | | | |
| 295 | 1-methoxy-3-[4-(4-morpholin-4-yl-1-phenyl-1H- | 446.2 | 2.31 | 14 |
| | pyrazolo[3,4-d]pyrimidin-6-yl)phenyl]urea | | | |
| 296 | 1-(allyloxy)-3-[4-(4-morpholin-4-yl-1-phenyl-1H- | 472.2 | 2.39 | 14 |
| | pyrazolo[3,4-d]pyrimidin-6-yl)phenyl]urea | | | |
| 297 | 1-methyl-3-[4-(4-morpholin-4-yl-1-phenyl-1H- | 430.2 | 2.29 | 14 |
| | pyrazolo[3,4-d]pyrimidin-6-yl)phenyl]urea | | | |
| 298 | 4-[1-(1-benzoylpiperidin-4-yl)-4-morpholin-4-yl-1H- | 484.24628¹ | | 3 |
| | pyrazolo[3,4-d]pyrimidin-6-yl]aniline | | | |
| 299 | N-{4-[1-(1-benzylpiperidin-4-yl)-4-morpholin-4-yl-1H- | 566.2 | 2.17 | 22 |
| | pyrazolo[3,4-d]pyrimidin-6-yl]phenyl}-2,2,2- | | | |
| | trifluoroacetamide | | | |
| 300 | 1-{4-[1-(1-benzylpiperidin-4-yl)-4-morpholin-4-yl-1H- | 570.3 | 1.66 | 14, then |
| | pyrazolo[3,4-d]pyrimidin-6-yl]phenyl}-3-[2- | | | 12 |
| | (methylamino)ethyl]urea | | | |
| 301 | 1-(4-{4-morpholin-4-yl-1-[1-(pyridin-3- | 528.2 | 2 | 14 |
| | ylcarbonyl)piperidin-4-yl]-1H-pyrazolo[3,4-d]pyrimidin- | | | |
| | 6-yl}phenyl)urea | | | |
| 302 | 1-(4-{4-morpholin-4-yl-1-[1-(pyridin-3- | 605.3 | 1.98 | 14 |
| | ylcarbonyl)piperidin-4-yl]-1H-pyrazolo[3,4-d]pyrimidin- | | | |
| | 6-yl}phenyl)-3-pyridin-3-ylurea | | | |
| 303 | 1-(2-fluoroethyl)-3-(4-{4-morpholin-4-yl-1-[1-(pyridin-3- | 574.3 | 2.14 | 14 |
| | ylcarbonyl)piperidin-4-yl]-1H-pyrazolo[3,4-d]pyrimidin- | | | |
| | 6-yl}phenyl)urea | | | |
| 304 | 1-(2-hydroxyethyl)-3-(4-{4-morpholin-4-yl-1-[1-(pyridin- | 572.3 | 1.98 | 14 |
| | 3-ylcarbonyl)piperidin-4-yl]-1H-pyrazolo[3,4- | | | |
| | d]pyrimidin-6-yl}phenyl)urea | | | |
| 305 | 1-hydroxy-3-(4-{4-morpholin-4-yl-1-[1-(pyridin-3- | 544.2 | 1.96 | 14 |
| | ylcarbonyl)piperidin-4-yl]-1H-pyrazolo[3,4-d]pyrimidin- | | | |
| | 6-yl}phenyl)urea | | | |
| 306 | N-(4-{4-morpholin-4-yl-1-[1-(pyridin-3- | 543.3 | 1.95 | 14 |
| | ylcarbonyl)piperidin-4-yl]-1H-pyrazolo[3,4-d]pyrimidin- | | | |
| | 6-yl}phenyl)hydrazinecarboxamide | | | |
| 307 | 1-ethyl-3-(4-{4-morpholin-4-yl-1-[1-(pyridin-3- | 556.3 | 2.16 | 14 |
| | ylcarbonyl)piperidin-4-yl]-1H-pyrazolo[3,4-d]pyrimidin- | | | |
| | 6-yl}phenyl)urea | | | |
| 308 | 1-methoxy-3-(4-{4-morpholin-4-yl-1-[1-(pyridin-3- | 558.3 | 2.13 | 14 |
| | ylcarbonyl)piperidin-4-yl]-1H-pyrazolo[3,4-d]pyrimidin- | | | |
| | 6-yl}phenyl)urea | | | |
| 309 | N-{4-[1-(1-benzylpiperidin-4-yl)-4-morpholin-4-yl-1H- | 528.3 | 1.83 | 14 |
| | pyrazolo[3,4-d]pyrimidin-6- | | | |
| | yl]phenyl}hydrazinecarboxamide | | | |
| 310 | 1-{4-[1-(1-benzylpiperidin-4-yl)-4-morpholin-4-yl-1H- | 529.3 | 1.84 | 14 |
| | pyrazolo[3,4-d]pyrimidin-6-yl]phenyl}-3-hydroxyurea | | | |
| 311 | 1-{4-[1-(1-benzylpiperidin-4-yl)-4-morpholin-4-yl-1H- | 553.3 | 2 | 14 |
| | pyrazolo[3,4-d]pyrimidin-6-yl]phenyl}-3- | | | |
| | cyclopropylurea | | | |
| 312 | 1-{4-[1-(1-benzylpiperidin-4-yl)-4-morpholin-4-yl-1H- | 551.3 | 1.98 | 14 |
| | pyrazolo[3,4-d]pyrimidin-6-yl]phenyl}-3-prop-2-yn-1- | | | |
| | ylurea | | | |
| 313 | 1-(4-{4-morpholin-4-yl-1-[1-(pyridin-3- | 514.3 | 1.74 | 14 |
| | ylmethyl)piperidin-4-yl]-1H-pyrazolo[3,4-d]pyrimidin-6- | | | |
| | yl}phenyl)urea | | | |
| 314 | 1-(4-{4-morpholin-4-yl-1-[1-(pyridin-3- | 591.3 | 1.71 | 14 |
| | ylmethyl)piperidin-4-yl]-1H-pyrazolo[3,4-d]pyrimidin-6- | | | |
| | yl}phenyl)-3-pyridin-3-ylurea | | | |
| 315 | 1-(2-fluoroethyl)-3-(4-{4-morpholin-4-yl-1-[1-(pyridin-3- | 560.3 | 1.83 | 14 |
| | ylmethyl)piperidin-4-yl]-1H-pyrazolo[3,4-d]pyrimidin-6- | | | |
| | yl}phenyl)urea | | | |
| 316 | 1-(2-hydroxyethyl)-3-(4-{4-morpholin-4-yl-1-[1-(pyridin- | 558.3 | 1.72 | 14 |
| | 3-ylmethyl)piperidin-4-yl]-1H-pyrazolo[3,4-d]pyrimidin- | | | |
| | 6-yl}phenyl)urea | | | |
| 317 | 1-hydroxy-3-(4-{4-morpholin-4-yl-1-[1-(pyridin-3- | 530.3 | 1.7 | 14 |
| | ylmethyl)piperidin-4-yl]-1H-pyrazolo[3,4-d]pyrimidin-6- | | | |
| | yl}phenyl)urea | | | |
| 318 | 1-ethyl-3-(4-{4-morpholin-4-yl-1-[1-(pyridin-3- | 542.3 | 1.85 | 14 |
| | ylmethyl)piperidin-4-yl]-1H-pyrazolo[3,4-d]pyrimidin-6- | | | |
| | yl}phenyl)urea | | | |
| 319 | 1-methoxy-3-(4-{4-morpholin-4-yl-1-[1-(pyridin-3- | 544.3 | 1.81 | 14 |
| | ylmethyl)piperidin-4-yl]-1H-pyrazolo[3,4-d]pyrimidin-6- | | | |
| | yl}phenyl)urea | | | |
| 320 | 4-[1-(1,4-dioxaspiro[4.5]dec-8-yl)-4-morpholin-4-yl-1H- | 437.2 | 2.14 | 3 |
| | pyrazolo[3,4-d]pyrimidin-6-yl]aniline | | | |
| 321 | 1-{4-[1-(1,4-dioxaspiro[4.5]dec-8-yl)-4-morpholin-4-yl- | 494.2 | 2.19 | 14 |
| | 1H-pyrazolo[3,4-d]pyrimidin-6-yl]phenyl}-3-methylurea | | | |
| 322 | 1-methyl-3-{4-[4-morpholin-4-yl-1-(4-oxocyclohexyl)- | 450.2 | 2.06 | 17 |
| | 1H-pyrazolo[3,4-d]pyrimidin-6-yl]phenyl}urea | | | |
| 323 | 1-{4-[1-(4-hydroxycyclohexyl)-4-morpholin-4-yl-1H- | 452.2 | 2 | 17 |
| | pyrazolo[3,4-d]pyrimidin-6-yl]phenyl}-3-methylurea | | | |
| 324 | tert-butyl 4-[6-(4-aminophenyl)-4-morpholin-4-yl-1H- | 480.27325¹ | | 3 |
| | pyrazolo[3,4-d]pyrimidin-1-yl]piperidine-1-carboxylate | | | |
| 325 | 1-{4-[1-(1-benzylpiperidin-4-yl)-4-morpholin-4-yl-1H- | 543.3 | 1.95 | 16 |
| | pyrazolo[3,4-d]pyrimidin-6-yl]phenyl}-3-methylthiourea | | | |
| 326 | 1-{4-[1-(1-benzylpiperidin-4-yl)-4-morpholin-4-yl-1H- | 597.3 | 1.77 | 14 |
| | pyrazolo[3,4-d]pyrimidin-6-yl]phenyl}-3-(1H-imidazol- | | | |
| | 2-yl)urea | | | |
| 327 | 5-[1-(1-benzylpiperidin-4-yl)-4-morpholin-4-yl-1H- | 511.2 | 1.84 | 3 |
| | pyrazolo[3,4-d]pyrimidin-6-yl]-1,3-dihydro-2H- | | | |
| | benzimidazol-2-one | | | |
| 328 | 1-methyl-3-[4-(4-morpholin-4-yl-1-{1-[(1-oxidopyridin- | 558.3 | 1.98 | 7 |
| | 3-yl)carbonyl]piperidin-4-yl}-1H-pyrazolo[3,4- | | | |
| | d]pyrimidin-6-yl)phenyl]urea | | | |
| 329 | 1-methyl-3-[4-(1-{1-[(2-methylpyridin-3- | 556.3 | 1.97 | 7 |
| | yl)carbonyl]piperidin-4-yl}-4-morpholin-4-yl-1H- | | | |
| | pyrazolo[3,4-d]pyrimidin-6-yl)phenyl]urea | | | |
| 330 | 1-[4-(1-{1-[(6-methoxypyridin-3-yl)methyl]piperidin-4- | 558.3 | 1.86 | 8 |
| | yl}-4-morpholin-4-yl-1H-pyrazolo[3,4-d]pyrimidin-6- | | | |
| | yl)phenyl]-3-methylurea | | | |
| 331 | 1-methyl-3-(4-{4-morpholin-4-yl-1-[1-(pyridin-2- | 528.3 | 1.84 | 8 |
| | ylmethyl)piperidin-4-yl]-1H-pyrazolo[3,4-d]pyrimidin-6- | | | |
| | yl}phenyl)urea | | | |
| 332 | 2-[4-(6-{4-[(methylcarbamoyl)amino]phenyl}-4- | 494.3 | 1.69 | 23 |
| | morpholin-4-yl-1H-pyrazolo[3,4-d]pyrimidin-1- | | | |
| | yl)piperidin-1-yl]acetamide | | | |
| 333 | 1-methyl-3-(4-{4-morpholin-4-yl-1-[1-(2-oxo-2- | 555.3 | 1.91 | 23 |
| | phenylethyl)piperidin-4-yl]-1H-pyrazolo[3,4- | | | |
| | d]pyrimidin-6-yl}phenyl)urea | | | |
| 334 | 1-methyl-3-[4-(1-{1-[(4-methylpiperazin-1- | 563.3 | 1.8 | 7 |
| | yl)carbonyl]piperidin-4-yl}-4-morpholin-4-yl-1H- | | | |
| | pyrazolo[3,4-d]pyrimidin-6-yl)phenyl]urea | | | |
| 335 | 4-(6-{4-[(methylcarbamoyl)amino]phenyl}-4-morpholin- | 557.3 | 1.85 | 7 |
| | 4-yl-1H-pyrazolo[3,4-d]pyrimidin-1-yl)-N-pyridin-3- | | | |
| | ylpiperidine-1-carboxamide | | | |
| 336 | 1-{4-[1-(1-benzoylpiperidin-4-yl)-4-morpholin-4-yl-1H- | 541.3 | 2.23 | 7 |
| | pyrazolo[3,4-d]pyrimidin-6-yl]phenyl}-3-methylurea | | | |
| 337 | tert-butyl 4-(6-{4-[(methylcarbamoyl)amino]phenyl}-4- | 537.3 | 2.37 | 14 |
| | morpholin-4-yl-1H-pyrazolo[3,4-d]pyrimidin-1- | | | |
| | yl)piperidine-1-carboxylate | | | |
| 338 | 1-methyl-3-[4-(4-morpholin-4-yl-1-piperidin-4-yl-1H- | 437.2 | 1.69 | 6 |
| | pyrazolo[3,4-d]pyrimidin-6-yl)phenyl]urea | | | |
| 339 | 1-(4-{4-morpholin-4-yl-1-[1-(pyridin-3- | 590.3 | 2.07 | 14 |
| | ylmethyl)piperidin-4-yl]-1H-pyrazolo[3,4-d]pyrimidin-6- | | | |
| | yl}phenyl)-3-phenylurea | | | |
| 340 | 1-(4-{4-morpholin-4-yl-1-[1-(pyridin-3- | 591.3 | 1.7 | 14 |
| | ylmethyl)piperidin-4-yl]-1H-pyrazolo[3,4-d]pyrimidin-6- | | | |
| | yl}phenyl)-3-pyridin-4-ylurea | | | |
| 341 | 1-(4-{4-morpholin-4-yl-1-[1-(pyridin-3- | 591.3 | 2.1 | 14 |
| | ylmethyl)piperidin-4-yl]-1H-pyrazolo[3,4-d]pyrimidin-6- | | | |
| | yl}phenyl)-3-pyridin-2-ylurea | | | |
| 342 | 1-[2-(methylamino)ethyl]-3-(4-{4-morpholin-4-yl-1-[1- | 571.3 | 1.59 | 14, then |
| | (pyridin-3-ylmethyl)piperidin-4-yl]-1H-pyrazolo[3,4- | | | 12 |
| | d]pyrimidin-6-yl}phenyl)urea | | | |
| 343 | 1-(4-{4-morpholin-4-yl-1-[1-(pyridin-3- | 604.3 | 2.37 | 14 |
| | ylcarbonyl)piperidin-4-yl]-1H-pyrazolo[3,4-d]pyrimidin- | | | |
| | 6-yl}phenyl)-3-phenylurea | | | |
| 344 | 1-(4-{4-morpholin-4-yl-1-[1-(pyridin-3- | 605.3 | 1.99 | 14 |
| | ylcarbonyl)piperidin-4-yl]-1H-pyrazolo[3,4-d]pyrimidin- | | | |
| | 6-yl}phenyl)-3-pyridin-4-ylurea | | | |
| 345 | 1-(4-{4-morpholin-4-yl-1-[1-(pyridin-3- | 605.3 | 2.44 | 14 |
| | ylcarbonyl)piperidin-4-yl]-1H-pyrazolo[3,4-d]pyrimidin- | | | |
| | 6-yl}phenyl)-3-pyridin-2-ylurea | | | |
| 346 | 1-[2-(methylamino)ethyl]-3-(4-{4-morpholin-4-yl-1-[1- | 585.3 | 1.81 | 14, then |
| | (pyridin-3-ylcarbonyl)piperidin-4-yl]-1H-pyrazolo[3,4- | | | 12 |
| | d]pyrimidin-6-yl}phenyl)urea | | | |
| 347 | 4-[1-(1-benzylpiperidin-4-yl)-4-morpholin-4-yl-1H- | 488.2 | 1.98 | 3 |
| | pyrazolo[3,4-d]pyrimidin-6-yl]-2-fluoroaniline | | | |
| 348 | 1-{4-[1-(1-benzylpiperidin-4-yl)-4-morpholin-4-yl-1H- | 545.3 | 1.93 | 14 |
| | pyrazolo[3,4-d]pyrimidin-6-yl]-2-fluorophenyl}-3- | | | |
| | methylurea | | | |
| 349 | 1-{4-[1-(1-benzylpiperidin-4-yl)-4-morpholin-4-yl-1H- | 559.3 | 2.03 | 14 |
| | pyrazolo[3,4-d]pyrimidin-6-yl]-2-fluorophenyl}-3- | | | |
| | ethylurea | | | |
| 350 | 1-(2-fluoro-4-{4-morpholin-4-yl-1-[1-(pyridin-3- | 546.3 | 1.84 | 14 |
| | ylmethyl)piperidin-4-yl]-1H-pyrazolo[3,4-d]pyrimidin-6- | | | |
| | yl}phenyl)-3-methylurea | | | |
| 351 | 1-(2-fluoro-4-{4-morpholin-4-yl-1-[1-(pyridin-3- | 608.3 | 2.14 | 14 |
| | ylmethyl)piperidin-4-yl]-1H-pyrazolo[3,4-d]pyrimidin-6- | | | |
| | yl}phenyl)-3-phenylurea | | | |
| 352 | 1-(2-fluoro-4-{4-morpholin-4-yl-1-[1-(pyridin-3- | 609.3 | 1.74 | 14 |
| | ylmethyl)piperidin-4-yl]-1H-pyrazolo[3,4-d]pyrimidin-6- | | | |
| | yl}phenyl)-3-pyridin-4-ylurea | | | |
| 353 | {4-[1-(1-benzylpiperidin-4-yl)-4-morpholin-4-yl-1H- | 513.25891¹ | | 3 |
| | pyrazolo[3,4-d]pyrimidin-6-yl]phenyl}acetic acid | | | |
| 354 | 1-(4-{1-[1-(2-fluorobenzoyl)piperidin-4-yl]-4-morpholin- | 559.3 | 2.19 | 7 |
| | 4-yl-1H-pyrazolo[3,4-d]pyrimidin-6-yl}phenyl)-3- | | | |
| | methylurea | | | |
| 355 | 1-(4-{1-[1-(2-chlorobenzoyl)piperidin-4-yl]-4-morpholin- | 575.2 | 2.24 | 7 |
| | 4-yl-1H-pyrazolo[3,4-d]pyrimidin-6-yl}phenyl)-3- | | | |
| | methylurea | | | |
| 356 | 1-methyl-3-(4-{1-[1-(2-methylbenzoyl)piperidin-4-yl]-4- | 555.3 | 2.24 | 7 |
| | morpholin-4-yl-1H-pyrazolo[3,4-d]pyrimidin-6- | | | |
| | yl}phenyl)urea | | | |
| 357 | 1-(4-{1-[1-(3-fluorobenzoyl)piperidin-4-yl]-4-morpholin- | 559.3 | 2.2 | 7 |
| | 4-yl-1H-pyrazolo[3,4-d]pyrimidin-6-yl}phenyl)-3- | | | |
| | methylurea | | | |
| 358 | 1-(4-{1-[1-(3-chlorobenzoyl)piperidin-4-yl]-4-morpholin- | 575.2 | 2.28 | 7 |
| | 4-yl-1H-pyrazolo[3,4-d]pyrimidin-6-yl}phenyl)-3- | | | |
| | methylurea | | | |
| 359 | 1-methyl-3-[4-(4-morpholin-4-yl-1-{1-[3- | 609.2 | 2.3 | 7 |
| | (trifluoromethyl)benzoyl]piperidin-4-yl}-1H- | | | |
| | pyrazolo[3,4-d]pyrimidin-6-yl)phenyl]urea | | | |
| 360 | 1-(4-{1-[1-(4-bromobenzoyl)piperidin-4-yl]-4-morpholin- | 619.2 | 2.31 | 7 |
| | 4-yl-1H-pyrazolo[3,4-d]pyrimidin-6-yl}phenyl)-3- | | | |
| | methylurea | | | |
| 361 | 1-(4-{1-[1-(4-fluorobenzoyl)piperidin-4-yl]-4-morpholin- | 559.3 | 2.2 | 7 |
| | 4-yl-1H-pyrazolo[3,4-d]pyrimidin-6-yl}phenyl)-3- | | | |
| | methylurea | | | |
| 362 | 1-(4-{1-[1-(4-chlorobenzoyl)piperidin-4-yl]-4-morpholin- | 575.2 | 2.28 | 7 |
| | 4-yl-1H-pyrazolo[3,4-d]pyrimidin-6-yl}phenyl)-3- | | | |
| | methylurea | | | |
| 363 | 1-(4-{1-[1-(4-methoxybenzoyl)piperidin-4-yl]-4- | 571.3 | 2.2 | 7 |
| | morpholin-4-yl-1H-pyrazolo[3,4-d]pyrimidin-6- | | | |
| | yl}phenyl)-3-methylurea | | | |
| 364 | 1-(4-{1-[1-(3-methoxybenzoyl)piperidin-4-yl]-4- | 571.3 | 2.2 | 7 |
| | morpholin-4-yl-1H-pyrazolo[3,4-d]pyrimidin-6- | | | |
| | yl}phenyl)-3-methylurea | | | |
| 365 | 1-(4-{1-[1-(2-methoxybenzoyl)piperidin-4-yl]-4- | 571.3 | 2.19 | 24 |
| | morpholin-4-yl-1H-pyrazolo[3,4-d]pyrimidin-6- | | | |
| | yl}phenyl)-3-methylurea | | | |
| 366 | 1-methyl-3-(4-{1-[1-(3-methylbenzoyl)piperidin-4-yl]-4- | 555.3 | 2.27 | 14 |
| | morpholin-4-yl-1H-pyrazolo[3,4-d]pyrimidin-6- | | | |
| | yl}phenyl)urea | | | |
| 367 | 1-methyl-3-(4-{1-[1-(4-methylbenzoyl)piperidin-4-yl]-4- | 555.3 | 2.26 | 14 |
| | morpholin-4-yl-1H-pyrazolo[3,4-d]pyrimidin-6- | | | |
| | yl}phenyl)urea | | | |
| 368 | 1-(4-{1-[1-(4-cyanobenzoyl)piperidin-4-yl]-4-morpholin- | Not | | 14 |
| | 4-yl-1H-pyrazolo[3,4-d]pyrimidin-6-yl}phenyl)-3- | Determined | | |
| | methylurea | | | |
| 369 | 6-[1-(1-benzylpiperidin-4-yl)-4-morpholin-4-yl-1H- | 506.3 | | 15 |
| | pyrazolo[3,4-d]pyrimidin-6-yl]quinoline | | | |
| 370 | 2-{4-[1-(1-benzylpiperidin-4-yl)-4-morpholin-4-yl-1H- | 512.3 | 1.83 | 15 |
| | pyrazolo[3,4-d]pyrimidin-6-yl]phenyl}acetamide | | | |
| 371 | 2-{4-[1-(1-benzylpiperidin-4-yl)-4-morpholin-4-yl-1H- | 526.3 | 1.86 | 16 |
| | pyrazolo[3,4-d]pyrimidin-6-yl]phenyl}-N- | | | |
| | methylacetamide | | | |
| 372 | 1-ethyl-3-(2-fluoro-4-{4-morpholin-4-yl-1-[1-(pyridin-3- | 560.3 | 1.9 | 14 |
| | ylmethyl)piperidin-4-yl]-1H-pyrazolo[3,4-d]pyrimidin-6- | | | |
| | yl}phenyl)urea | | | |
| 373 | 1-(2-fluoro-4-{4-morpholin-4-yl-1-[1-(pyridin-3- | 609.3 | 1.78 | 14 |
| | ylmethyl)piperidin-4-yl]-1H-pyrazolo[3,4-d]pyrimidin-6- | | | |
| | yl}phenyl)-3-pyridin-3-ylurea | | | |
| 374 | 1-(2-fluoroethyl)-3-(2-fluoro-4-{4-morpholin-4-yl-1-[1- | ND | | 14 |
| | (pyridin-3-ylmethyl)piperidin-4-yl]-1H-pyrazolo[3,4- | | | |
| | d]pyrimidin-6-yl}phenyl)urea | | | |
| 375 | 1-(4-{4-morpholin-4-yl-1-[1-(pyridin-3- | 596.2 | 1.99 | 14 |
| | ylmethyl)piperidin-4-yl]-1H-pyrazolo[3,4-d]pyrimidin-6- | | | |
| | yl}phenyl)-3-(3-thienyl)urea | | | |
| 376 | 1-(2-furylmethyl)-3-(4-{4-morpholin-4-yl-1-[1-(pyridin- | 594.3 | 1.91 | 14 |
| | 3-ylmethyl)piperidin-4-yl]-1H-pyrazolo[3,4-d]pyrimidin- | | | |
| | 6-yl}phenyl)urea | | | |
| 377 | 1-methyl-3-(4-{4-morpholin-4-yl-1-[1-(pyridin-3- | 544.3 | 1.78 | 14 |
| | ylmethyl)piperidin-4-yl]-1H-pyrazolo[3,4-d]pyrimidin-6- | | | |
| | yl}phenyl)thiourea | | | |
| 378 | 1-{4-[1-(1-benzoylpiperidin-4-yl)-4-morpholin-4-yl-1H- | 603.3 | 2.43 | 14 |
| | pyrazolo[3,4-d]pyrimidin-6-yl]phenyl}-3-phenylurea | | | |
| 379 | 1-{4-[1-(1-benzoylpiperidin-4-yl)-4-morpholin-4-yl-1H- | 604.3 | 2.09 | 14 |
| | pyrazolo[3,4-d]pyrimidin-6-yl]phenyl}-3-pyridin-3-ylurea | | | |
| 380 | 1-{4-[1-(1-benzoylpiperidin-4-yl)-4-morpholin-4-yl-1H- | 573.5 | 2.19 | 14, then |
| | pyrazolo[3,4-d]pyrimidin-6-yl]phenyl}-3-(2- | | | 12 |
| | fluoroethyl)urea | | | |
| 381 | 1-{4-[1-(1-benzoylpiperidin-4-yl)-4-morpholin-4-yl-1H- | 555.3 | 2.24 | 8 |
| | pyrazolo[3,4-d]pyrimidin-6-yl]phenyl}-3-ethylurea | | | |
| 382 | 1-{4-[1-(1-benzoylpiperidin-4-yl)-4-morpholin-4-yl-1H- | 527.2 | 2.12 | 8 |
| | pyrazolo[3,4-d]pyrimidin-6-yl]phenyl}urea | | | |
| 383 | ethyl {4-[1-(1-benzoylpiperidin-4-yl)-4-morpholin-4-yl- | 556.3 | 2.42 | 8 |
| | 1H-pyrazolo[3,4-d]pyrimidin-6-yl]phenyl}carbamate | | | |
| 384 | 1-{4-[1-(1-benzoylpiperidin-4-yl)-4-morpholin-4-yl-1H- | 604.3 | 2.06 | 8 |
| | pyrazolo[3,4-d]pyrimidin-6-yl]phenyl}-3-pyridin-4-ylurea | | | |
| 385 | 1-{4-[1-(1-benzoylpiperidin-4-yl)-4-morpholin-4-yl-1H- | 571.3 | 2.1 | 8 |
| | pyrazolo[3,4-d]pyrimidin-6-yl]phenyl}-3-(2- | | | |
| | hydroxyethyl)urea | | | |
| 386 | 1-{4-[1-(1-benzoylpiperidin-4-yl)-4-morpholin-4-yl-1H- | 584.3 | 1.93 | 8 |
| | pyrazolo[3,4-d]pyrimidin-6-yl]phenyl}-3-[2- | | | |
| | (methylamino)ethyl]urea | | | |
| 387 | 1-[4-(1-{1-[(6-fluoropyridin-3-yl)methyl]piperidin-4-yl}- | 546.3 | 1.8 | 8 |
| | 4-morpholin-4-yl-1H-pyrazolo[3,4-d]pyrimidin-6- | | | |
| | yl)phenyl]-3-methylurea | | | |
| 388 | 1-[4-(1-{1-[(6-chloropyridin-3-yl)methyl]piperidin-4-yl}- | 562.2 | 1.84 | 8 |
| | 4-morpholin-4-yl-1H-pyrazolo[3,4-d]pyrimidin-6- | | | |
| | yl)phenyl]-3-methylurea | | | |
| 389 | 1-[4-(1-{1-[(6-bromopyridin-3-yl)methyl]piperidin-4-yl}- | 606.2 | 1.85 | 8 |
| | 4-morpholin-4-yl-1H-pyrazolo[3,4-d]pyrimidin-6- | | | |
| | yl)phenyl]-3-methylurea | | | |
| 390 | 1-[4-(1-{1-[(2-chloropyridin-3-yl)methyl]piperidin-4-yl}- | 562.2 | 1.83 | 8 |
| | 4-morpholin-4-yl-1H-pyrazolo[3,4-d]pyrimidin-6- | | | |
| | yl)phenyl]-3-methylurea | | | |
| 391 | 1-[4-(1-{1-[(4-methoxypyridin-3-yl)methyl]piperidin-4- | 558.3 | 1.84 | 8 |
| | yl}-4-morpholin-4-yl-1H-pyrazolo[3,4-d]pyrimidin-6- | | | |
| | yl)phenyl]-3-methylurea | | | |
| 392 | 1-[4-(1-{1-[(5-fluoropyridin-3-yl)methyl]piperidin-4-yl}- | 546.3 | 1.81 | 8 |
| | 4-morpholin-4-yl-1H-pyrazolo[3,4-d]pyrimidin-6- | | | |
| | yl)phenyl]-3-methylurea | | | |
| 393 | 1-[4-(1-{1-[(5-bromopyridin-3-yl)methyl]piperidin-4-yl}- | 606.2 | 1.86 | 8 |
| | 4-morpholin-4-yl-1H-pyrazolo[3,4-d]pyrimidin-6- | | | |
| | yl)phenyl]-3-methylurea | | | |
| 394 | 1-(4-{1-[1-(4-chlorobenzyl)piperidin-4-yl]-4-morpholin- | 561.2 | 1.98 | 8 |
| | 4-yl-1H-pyrazolo[3,4-d]pyrimidin-6-yl}phenyl)-3- | | | |
| | methylurea | | | |
| 395 | 1-(4-{1-[1-(3-chlorobenzyl)piperidin-4-yl]-4-morpholin- | 561.2 | 1.95 | 8 |
| | 4-yl-1H-pyrazolo[3,4-d]pyrimidin-6-yl}phenyl)-3- | | | |
| | methylurea | | | |
| 396 | 1-(4-{1-[1-(2-chlorobenzyl)piperidin-4-yl]-4-morpholin- | 561.2 | 1.96 | 8 |
| | 4-yl-1H-pyrazolo[3,4-d]pyrimidin-6-yl}phenyl)-3- | | | |
| | methylurea | | | |
| 397 | 1-(4-{1-[1-(3-hydroxybenzyl)piperidin-4-yl]-4- | 543.3 | 1.81 | 14 |
| | morpholin-4-yl-1H-pyrazolo[3,4-d]pyrimidin-6- | | | |
| | yl}phenyl)-3-methylurea | | | |
| 398 | 1-(4-{1-[1-(3-hydroxy-4-methoxybenzyl)piperidin-4-yl]- | 573.3 | 1.83 | 15 |
| | 4-morpholin-4-yl-1H-pyrazolo[3,4-d]pyrimidin-6- | | | |
| | yl}phenyl)-3-methylurea | | | |
| 399 | 1-(4-{1-[1-(2-hydroxybenzyl)piperidin-4-yl]-4- | 543.3 | 1.85 | 14 |
| | morpholin-4-yl-1H-pyrazolo[3,4-d]pyrimidin-6- | | | |
| | yl}phenyl)-3-methylurea | | | |
| 400 | 1-(4-{1-[1-(3-methoxybenzyl)piperidin-4-yl]-4- | 557.3 | 1.9 | 25 |
| | morpholin-4-yl-1H-pyrazolo[3,4-d]pyrimidin-6- | | | |
| | yl}phenyl)-3-methylurea | | | |
| 401 | 1-methyl-3-{4-[1-(1-methylpiperidin-4-yl)-4-morpholin- | 451.2 | 1.71 | 25 |
| | 4-yl-1H-pyrazolo[3,4-d]pyrimidin-6-yl]phenyl}urea | | | |
| 402 | 1-(4-{1-[1-(2-furylmethyl)piperidin-4-yl]-4-morpholin-4- | 517.3 | 1.81 | 25 |
| | yl-1H-pyrazolo[3,4-d]pyrimidin-6-yl}phenyl)-3- | | | |
| | methylurea | | | |
| 403 | 1-methyl-3-{4-[4-morpholin-4-yl-1-(tetrahydro-2H-pyran- | 438.22597¹ | | 25 |
| | 4-yl)-1H-pyrazolo[3,4-d]pyrimidin-6-yl]phenyl}urea | | | |
| 404 | 1-(4-{4-morpholin-4-yl-1-[1-(pyridin-3- | 596.2 | 1.97 | 25 |
| | ylmethyl)piperidin-4-yl]-1H-pyrazolo[3,4-d]pyrimidin-6- | | | |
| | yl}phenyl)-3-(2-thienyl)urea | | | |
| 405 | 1-cyclopropyl-3-(4-{4-morpholin-4-yl-1-[1-(pyridin-3- | 554.3 | 1.83 | 25 |
| | ylmethyl)piperidin-4-yl]-1H-pyrazolo[3,4-d]pyrimidin-6- | | | |
| | yl}phenyl)urea | | | |
| 406 | 2-cyano-1-(4-{4-morpholin-4-yl-1-[1-(pyridin-3- | 538.3 | 1.74 | 25 |
| | ylmethyl)piperidin-4-yl]-1H-pyrazolo[3,4-d]pyrimidin-6- | | | |
| | yl}phenyl)guanidine | | | |
| 407 | 2-cyano-1-methyl-3-(4-{4-morpholin-4-yl-1-[1-(pyridin- | 552.3 | 1.78 | 15 |
| | 3-ylmethyl)piperidin-4-yl]-1H-pyrazolo[3,4-d]pyrimidin- | | | |
| | 6-yl}phenyl)guanidine | | | |
| 408 | 2-cyano-1-ethyl-3-(4-{4-morpholin-4-yl-1-[1-(pyridin-3- | 566.3 | 1.81 | 15 |
| | ylmethyl)piperidin-4-yl]-1H-pyrazolo[3,4-d]pyrimidin-6- | | | |
| | yl}phenyl)guanidine | | | |
| 409 | N′-cyano-N-(4-{4-morpholin-4-yl-1-[1-(pyridin-3- | 539.3 | 1.77 | 3, then |
| | ylmethyl)piperidin-4-yl]-1H-pyrazolo[3,4-d]pyrimidin-6- | | | 14 |
| | yl}phenyl)carbamimidic acid | | | |
| 410 | methyl N′-cyano-N-(4-{4-morpholin-4-yl-1-[1-(pyridin-3- | 553.3 | 1.86 | 3 |
| | ylmethyl)piperidin-4-yl]-1H-pyrazolo[3,4-d]pyrimidin-6- | | | |
| | yl}phenyl)imidocarbamate | | | |
| 411 | 2-cyano-1-(4-{4-morpholin-4-yl-1-[1-(pyridin-3- | 552.3 | 1.97 | 7 |
| | ylcarbonyl)piperidin-4-yl]-1H-pyrazolo[3,4-d]pyrimidin- | | | |
| | 6-yl}phenyl)guanidine | | | |
| 412 | 2-cyano-1-methyl-3-(4-{4-morpholin-4-yl-1-[1-(pyridin- | 566.3 | 1.98 | 7 |
| | 3-ylcarbonyl)piperidin-4-yl]-1H-pyrazolo[3,4- | | | |
| | d]pyrimidin-6-yl}phenyl)guanidine | | | |
| 413 | 1-(4-{4-morpholin-4-yl-1-[1-(pyridin-3- | 610.2 | 2.23 | 7 |
| | ylcarbonyl)piperidin-4-yl]-1H-pyrazolo[3,4-d]pyrimidin- | | | |
| | 6-yl}phenyl)-3-(2-thienyl)urea | | | |
| 414 | 1-(4-{4-morpholin-4-yl-1-[1-(pyridin-3- | 610.2 | 2.25 | 7 |
| | ylcarbonyl)piperidin-4-yl]-1H-pyrazolo[3,4-d]pyrimidin- | | | |
| | 6-yl}phenyl)-3-(3-thienyl)urea | | | |
| 415 | 1-cyclopropyl-3-(4-{4-morpholin-4-yl-1-[1-(pyridin-3- | 568.3 | 2.07 | 7 |
| | ylcarbonyl)piperidin-4-yl]-1H-pyrazolo[3,4-d]pyrimidin- | | | |
| | 6-yl}phenyl)urea | | | |
| 416 | 6-(2,3-dihydro-1H-indol-5-yl)-4-morpholin-4-yl-1-[1- | 497.3 | 1.64 | 7 |
| | (pyridin-3-ylmethyl)piperidin-4-yl]-1H-pyrazolo[3,4- | | | |
| | d]pyrimidine | | | |
| 417 | 1-{4-[1-(1-isonicotinoylpiperidin-4-yl)-4-morpholin-4-yl- | 542.3 | 2.08 | 7 |
| | 1H-pyrazolo[3,4-d]pyrimidin-6-yl]phenyl}-3-methylurea | | | |
| 418 | 1-methyl-3-(4-{4-morpholin-4-yl-1-[1-(pyridin-2- | 542.3 | 2.18 | 7 |
| | ylcarbonyl)piperidin-4-yl]-1H-pyrazolo[3,4-d]pyrimidin- | | | |
| | 6-yl}phenyl)urea | | | |
| 419 | 1-methyl-3-[4-(1-{1-[(4-methylpyridin-3- | 556.3 | 2.04 | 7 |
| | yl)carbonyl]piperidin-4-yl}-4-morpholin-4-yl-1H- | | | |
| | pyrazolo[3,4-d]pyrimidin-6-yl)phenyl]urea | | | |
| 420 | 1-methyl-3-[4-(1-{1-[(6-methylpyridin-3- | 556.3 | 2.05 | 8 |
| | yl)carbonyl]piperidin-4-yl}-4-morpholin-4-yl-1H- | | | |
| | pyrazolo[3,4-d]pyrimidin-6-yl)phenyl]urea | | | |
| 421 | 1-[4-(1-{1-[(6-fluoropyridin-3-yl)carbonyl]piperidin-4- | 560.2 | 2.2 | 8 |
| | yl}-4-morpholin-4-yl-1H-pyrazolo[3,4-d]pyrimidin-6-yl) | | | |
| | phenyl]-3-methylurea | | | |
| 422 | 1-methyl-3-(4-{4-morpholin-4-yl-1-[1-(pyrazin-2- | 543.3 | 2.11 | 8 |
| | ylcarbonyl)piperidin-4-yl]-1H-pyrazolo[3,4-d]pyrimidin- | | | |
| | 6-yl}phenyl)urea | | | |
| 423 | 1-(4-{1-[1-(3-acetylbenzoyl)piperidin-4-yl]-4-morpholin- | 583.3 | 2.27 | 8 |
| | 4-yl-1H-pyrazolo[3,4-d]pyrimidin-6-yl}phenyl)-3- | | | |
| | methylurea | | | |
| 424 | 1-[4-(1-{1-[(6-chloropyridin-3-yl)carbonyl]piperidin-4- | 576.2 | 2.28 | 8 |
| | yl}-4-morpholin-4-yl-1H-pyrazolo[3,4-d]pyrimidin-6-yl) | | | |
| | phenyl]-3-methylurea | | | |
| 425 | 1-[4-(1-{1-[(2-chloropyridin-3-yl)carbonyl]piperidin-4- | 576.2 | 2.23 | 8 |
| | yl}-4-morpholin-4-yl-1H-pyrazolo[3,4-d]pyrimidin-6-yl) | | | |
| | phenyl]-3-methylurea | | | |
| 426 | 1-methyl-3-(4-{4-morpholin-4-yl-1-[1-(pyridin-4- | 528.3 | 1.72 | 3 |
| | ylmethyl)piperidin-4-yl]-1H-pyrazolo[3,4-d]pyrimidin-6- | | | |
| | yl}phenyl)urea | | | |
| 427 | 1-(4-{1-[1-(4-fluorobenzyl)piperidin-4-yl]-4-morpholin-4- | 545.3 | 1.89 | 3 |
| | yl-1H-pyrazolo[3,4-d] pyrimidin-6-yl}phenyl)-3- | | | |
| | methylurea | | | |
| 428 | 1-(4-{1-[1-(3-fluorobenzyl)piperidin-4-yl]-4-morpholin-4- | 545.3 | 1.89 | 3 |
| | yl-1H-pyrazolo[3,4-d] pyrimidin-6-yl}phenyl)-3- | | | |
| | methylurea | | | |
| 429 | 1-methyl-3-(4-{1-[1-(2-methylbenzyl)piperidin-4-yl]-4- | 541.3 | 1.92 | 3 |
| | morpholin-4-yl-1H-pyrazolo[3,4-d]pyrimidin-6-yl} | | | |
| | phenyl)urea | | | |
| 430 | 1-methyl-3-(4-{1-[1-(3-methylbenzyl)piperidin-4-yl]-4- | 541.3 | 1.94 | 3 |
| | morpholin-4-yl-1H-pyrazolo[3,4-d]pyrimidin-6-yl} | | | |
| | phenyl)urea | | | |
| 431 | 1-methyl-3-(4-{1-[1-(4-methylbenzyl)piperidin-4-yl]-4- | 541.3 | 1.94 | 3 |
| | morpholin-4-yl-1H-pyrazolo[3,4-d]pyrimidin-6-yl} | | | |
| | phenyl)urea | | | |
| 432 | 6-(1H-indazol-5-yl)-4-morpholin-4-yl-1-phenyl-1H- | 398.2 | 2.66 | 3 |
| | pyrazolo[3,4-d]pyrimidine | | | |
| 433 | 5-(4-morpholin-4-yl-1-phenyl-1H-pyrazolo[3,4- | 413.2 | 2.53 | 3 |
| | d]pyrimidin-6-yl)-1,3-dihydro-2H-indol-2-one | | | |
| 434 | 2-{4-morpholin-4-yl-1-[1-(pyridin-3- | 486.2 | 1.69 | 7 |
| | ylcarbonyl)piperidin-4-yl]-1H-pyrazolo[3,4-d]pyrimidin- | | | |
| | 6-yl}pyridin-4-amine | | | |
| 435 | 6-{4-morpholin-4-yl-1-[1-(pyridin-3- | 486.2 | 1.71 | 7 |
| | ylcarbonyl)piperidin-4-yl]-1H-pyrazolo[3,4-d]pyrimidin- | | | |
| | 6-yl}pyridin-3-amine | | | |
| 436 | 6-{4-morpholin-4-yl-1-[1-(pyridin-3- | 486.2 | 1.73 | 7 |
| | ylcarbonyl)piperidin-4-yl]-1H-pyrazolo[3,4-d]pyrimidin- | | | |
| | 6-yl}pyridin-2-amine | | | |
| 437 | 2-(4-morpholin-4-yl-1-phenyl-1H-pyrazolo[3,4- | 374.2 | 1.87 | 7 |
| | d]pyrimidin-6-yl)pyridin-4-amine | | | |
| 438 | 6-(4-morpholin-4-yl-1-phenyl-1H-pyrazolo[3,4- | 374.2 | 1.93 | 7 |
| | d]pyrimidin-6-yl)pyridin-3-amine | | | |
| 439 | 6-(4-morpholin-4-yl-1-phenyl-1H-pyrazolo[3,4- | 374.2 | 1.9 | 7 |
| | d]pyrimidin-6-yl)pyridin-2-amine | | | |
| 440 | methyl [4-(1-{1-[(4-methylpyridin-3- | 557.3 | 2.19 | 7 |
| | yl)carbonyl]piperidin-4-yl}-4-morpholin-4-yl-1H- | | | |
| | pyrazolo[3,4-d]pyrimidin-6-yl) phenyl]carbamate | | | |
| 441 | methyl (4-{4-morpholin-4-yl-1-[1-(pyridin-2- | 543.2 | 2.32 | 7 |
| | ylcarbonyl)piperidin-4-yl]-1H-pyrazolo[3,4-d]pyrimidin- | | | |
| | 6-yl}phenyl)carbamate | | | |
| 442 | methyl {4-[1-(1-benzoylpiperidin-4-yl)-4-morpholin-4-yl- | 542.2 | 2.44 | 7 |
| | 1H-pyrazolo[3,4-d]pyrimidin-6-yl]phenyl}carbamate | | | |
| 443 | methyl (4-{1-[1-(2-fluorobenzoyl)piperidin-4-yl]-4- | 560.2 | 2.44 | 7 |
| | morpholin-4-yl-1H-pyrazolo[3,4-d]pyrimidin-6-yl} | | | |
| | phenyl)carbamate | | | |
| 444 | methyl (4-{1-[1-(2-chlorobenzoyl)piperidin-4-yl]-4- | 576.2 | 2.49 | 8 |
| | morpholin-4-yl-1H-pyrazolo[3,4-d]pyrimidin-6-yl} | | | |
| | phenyl)carbamate | | | |
| 445 | methyl (4-{1-[1-(4-fluorobenzoyl)piperidin-4-yl]-4- | 560.2 | 2.44 | 8 |
| | morpholin-4-yl-1H-pyrazolo[3,4-d]pyrimidin-6-yl} | | | |
| | phenyl)carbamate | | | |
| 446 | methyl (4-{1-[1-(2-methoxybenzoyl)piperidin-4-yl]-4- | 572.3 | 2.44 | 8 |
| | morpholin-4-yl-1H-pyrazolo[3,4-d]pyrimidin-6-yl} | | | |
| | phenyl)carbamate | | | |
| 447 | methyl (4-{1-[1-(4-cyanobenzoyl)piperidin-4-yl]-4- | 567.3 | 2.34 | 8 |
| | morpholin-4-yl-1H-pyrazolo[3,4-d]pyrimidin-6-yl} | | | |
| | phenyl)carbamate | | | |
| 448 | methyl [4-(1-{1-[(4-methylpiperazin-1- | 564.3 | 1.95 | 8 |
| | yl)carbonyl]piperidin-4-yl}-4-morpholin-4-yl-1H- | | | |
| | pyrazolo[3,4-d]pyrimidin-6-yl)phenyl]carbamate | | | |
| 449 | methyl (4-{1-[1-(2,4-difluorobenzoyl)piperidin-4-yl]-4- | 578.2 | 2.46 | 8 |
| | morpholin-4-yl-1H-pyrazolo[3,4-d]pyrimidin-6-yl} | | | |
| | phenyl)carbamate | | | |
| 450 | methyl (4-{1-[1-(4-fluorobenzyl)piperidin-4-yl]-4- | Not Determined | | 23 |
| | morpholin-4-yl-1H-pyrazolo[3,4-d]pyrimidin-6-yl} | | | |
| | phenyl)carbamate | | | |
| 451 | methyl (4-{1-[1-(4-chlorobenzyl)piperidin-4-yl]-4- | 562.2 | 2.1 | 23 |
| | morpholin-4-yl-1H-pyrazolo[3,4-d]pyrimidin-6-yl} | | | |
| | phenyl)carbamate | | | |
| 452 | methyl [4-(1-{1-[(2-methoxypyridin-3- | 559.3 | 2 | 8 |
| | yl)methyl]piperidin-4-yl}-4-morpholin-4-yl-1H- | | | |
| | pyrazolo[3,4-d]pyrimidin-6-yl) phenyl]carbamate | | | |
| 453 | methyl [4-(1-{1-[(6-fluoropyridin-3-yl)methyl]piperidin- | 543.5 | 2.42 | 4 |
| | 4-yl}-4-morpholin-4-yl-1H-pyrazolo[3,4-d]pyrimidin-6- | | | |
| | yl) phenyl]carbamate | | | |
| 454 | methyl [4-(1-{1-[(6-chloropyridin-3-yl)methyl]piperidin- | 563.2 | 1.97 | 7 |
| | 4-yl}-4-morpholin-4-yl-1H-pyrazolo[3,4-d]pyrimidin-6- | | | |
| | yl) phenyl]carbamate | | | |
| 455 | methyl (4-{1-[1-(2-chlorobenzyl)piperidin-4-yl]-4- | 562.2 | 2.08 | 14 |
| | morpholin-4-yl-1H-pyrazolo[3,4-d]pyrimidin-6- | | | |
| | yl}phenyl)carbamate | | | |
| 456 | methyl (4-{1-[1-(2-amino-2-oxoethyl)piperidin-4-yl]-4- | 495.2 | 1.81 | 14 |
| | morpholin-4-yl-1H-pyrazolo[3,4-d]pyrimidin-6-yl} | | | |
| | phenyl)carbamate | | | |
| 457 | methyl (4-{4-morpholin-4-yl-1-[1-(2-oxo-2- | 556.3 | 2.02 | 14 |
| | phenylethyl)piperidin-4-yl]-1H-pyrazolo[3,4-d]pyrimidin- | | | |
| | 6-yl}phenyl)carbamate | | | |
| 458 | methyl {4-[1-(1-acryloylpiperidin-4-yl)-4-morpholin-4-yl- | 492.2 | 2.28 | 3 |
| | 1H-pyrazolo[3,4-d]pyrimidin-6-yl]phenyl}carbamate | | | |
| 459 | methyl [4-(4-morpholin-4-yl-1-piperidin-4-yl-1H- | 438.2 | 1.82 | 14 |
| | pyrazolo[3,4-d]pyrimidin-6-yl)phenyl]carbamate | | | |
| 460 | 3-fluoro-4-{4-morpholin-4-yl-1-[1-(pyridin-3- | 503.2 | 2.04 | 14 |
| | ylcarbonyl)piperidin-4-yl]-1H-pyrazolo[3,4-d]pyrimidin- | | | |
| | 6-yl}aniline | | | |
| 461 | 1-(3-fluoro-4-{4-morpholin-4-yl-1-[1-(pyridin-3- | 560.2 | 2.04 | 14 |
| | ylcarbonyl)piperidin-4-yl]-1H-pyrazolo[3,4-d]pyrimidin- | | | |
| | 6-yl}phenyl)-3-methylurea | | | |
| 462 | 1-ethyl-3-(3-fluoro-4-{4-morpholin-4-yl-1-[1-(pyridin-3- | 574.3 | 2.11 | 14 |
| | ylcarbonyl)piperidin-4-yl]-1H-pyrazolo[3,4-d]pyrimidin- | | | |
| | 6-yl}phenyl)urea | | | |
| 463 | 1-(2-fluoroethyl)-3-(3-fluoro-4-{4-morpholin-4-yl-1-[1- | 592.3 | 2.09 | 14 |
| | (pyridin-3-ylcarbonyl)piperidin-4-yl]-1H-pyrazolo[3,4- | | | |
| | d]pyrimidin-6-yl}phenyl)urea | | | |
| 464 | 2,5-difluoro-4-{4-morpholin-4-yl-1-[1-(pyridin-3- | 521.2 | 2.18 | 26 |
| | ylcarbonyl)piperidin-4-yl]-1H-pyrazolo[3,4-d]pyrimidin- | | | |
| | 6-yl}aniline | | | |
| 465 | 1-(2,5-difluoro-4-{4-morpholin-4-yl-1-[1-(pyridin-3- | 578.2 | 2.16 | 26 |
| | ylcarbonyl)piperidin-4-yl]-1H-pyrazolo[3,4-d]pyrimidin- | | | |
| | 6-yl}phenyl)-3-methylurea | | | |
| 466 | 1-(2,5-difluoro-4-{4-morpholin-4-yl-1-[1-(pyridin-3- | 592.3 | 2.24 | 7 |
| | ylcarbonyl)piperidin-4-yl]- | | | |
| | 1H-pyrazolo[3,4-d]pyrimidin-6-yl}phenyl)-3-ethylurea | | | |
| 467 | 1-(2,5-difluoro-4-{4-morpholin-4-yl-1-[1-(pyridin-3- | 610.2 | 2.24 | 7 |
| | ylcarbonyl)piperidin-4-yl]-1H-pyrazolo[3,4-d]pyrimidin- | | | |
| | 6-yl}phenyl)-3-(2-fluoroethyl)urea | | | |
| 468 | 1-cyclopropyl-3-(2,5-difluoro-4-{4-morpholin-4-yl-1-[1- | 604.3 | 2.29 | 7 |
| | (pyridin-3-ylcarbonyl)piperidin-4-yl]-1H-pyrazolo[3,4- | | | |
| | d]pyrimidin-6-yl}phenyl)urea | | | |
| 469 | 1-(2,5-difluoro-4-{4-morpholin-4-yl-1-[1-(pyridin-3- | 640.3 | 2.57 | 7 |
| | ylcarbonyl)piperidin-4-yl]-1H-pyrazolo[3,4-d]pyrimidin- | | | |
| | 6-yl}phenyl)-3-phenylurea | | | |
| 470 | 1-methyl-3-(4-{4-morpholin-4-yl-1-[1-(2,2,2- | 519.2 | 2.22 | 7 |
| | trifluoroethyl)piperidin-4-yl]-1H-pyrazolo[3,4- | | | |
| | d]pyrimidin-6-yl}phenyl)urea | | | |
| 471 | 6-(1H-indol-5-yl)-4-morpholin-4-yl-1-[1-(2,2,2- | 486.2 | 2.44 | 7 |
| | trifluoroethyl)piperidin-4-yl]-1H-pyrazolo[3,4- | | | |
| | d]pyrimidine | | | |
| 472 | 1-{4-[1-(1-acetylpiperidin-4-yl)-4-morpholin-4-yl-1H- | 479.2 | 2.07 | 27 |
| | pyrazolo[3,4-d]pyrimidin-6-yl]phenyl}-3-methylurea | | | |
| 473 | N,N-dimethyl-4-(6-{4- | 508.3 | 2.17 | 15 |
| | [(methylcarbamoyl)amino]phenyl}-4-morpholin-4-yl-1H- | | | |
| | pyrazolo[3,4-d]pyrimidin-1-yl)piperidine-1-carboxamide | | | |
| 474 | 1-(4-{1-[1-(methoxyacetyl)piperidin-4-yl]-4-morpholin-4- | 509.3 | 2.05 | 13, then 3 |
| | yl-1H-pyrazolo[3,4-d]pyrimidin-6-yl}phenyl)-3- | | | |
| | methylurea | | | |
| 475 | 1-{4-[1-(1-isobutyrylpiperidin-4-yl)-4-morpholin-4-yl- | 507.3 | 2.19 | 3 |
| | 1H-pyrazolo[3,4-d]pyrimidin-6-yl]phenyl}-3-methylurea | | | |
| 476 | methyl 4-(6-{4-[(methylcarbamoyl)amino]phenyl}-4- | 495.2 | 2.18 | 3 |
| | morpholin-4-yl-1H-pyrazolo[3,4-d]pyrimidin-1-yl) | | | |
| | piperidine-1-carboxylate | | | |
| 477 | N-methyl-4-(6-{4-[(methylcarbamoyl)amino]phenyl}-4- | 494.3 | 2.01 | 3 |
| | morpholin-4-yl-1H-pyrazolo[3,4-d]pyrimidin-1-yl) | | | |
| | piperidine-1-carboxamide | | | |
| 478 | 3-{4-[(2R,6S)-2,6-dimethylmorpholin-4-yl]-1-phenyl-1H- | 402.2 | 2.59 | 3 |
| | pyrazolo[3,4-d]pyrimidin-6-yl}phenol | | | |
| 479 | 1-ethyl-3-(5-{4-morpholin-4-yl-1-[1-(pyridin-3- | 543.3 | 1.87 | 21 |
| | ylmethyl)piperidin-4-yl]-1H-pyrazolo[3,4-d]pyrimidin-6- | | | |
| | yl}pyridin-2-yl)urea | | | |
| 480 | 1-methyl-3-(5-{4-morpholin-4-yl-1-[1-(pyridin-3- | 543.3 | 2.10 | 13 then 3 |
| | ylcarbonyl)piperidin-4-yl]-1H-pyrazolo[3,4-d]pyrimidin- | | | |
| | 6-yl}pyridin-2-yl)urea | | | |
| 481 | methyl {3-[1-(1-benzylpiperidin-4-yl)-4-morpholin-4-yl- | 528.3 | 2.03 | 29 |
| | 1H-pyrazolo[3,4-d]pyrimidin-6-yl]phenyl}carbamate | | | |
| 482 | N-{3-[1-(1-benzylpiperidin-4-yl)-4-morpholin-4-yl-1H- | 527.3 | 1.91 | 29 |
| | pyrazolo[3,4-d]pyrimidin-6-yl]phenyl}-N′-methylurea | | | |
| 483 | N-{3-[1-(1-benzylpiperidin-4-yl)-4-morpholin-4-yl-1H- | 513.3 | 1.91 | 29 |
| | pyrazolo[3,4-d]pyrimidin-6-yl]phenyl}urea | | | |
| 484 | 3-[1-(1-benzylpiperidin-4-yl)-4-morpholin-4-yl-1H- | 506.3 | 2.04 | 2 |
| | pyrazolo[3,4-d]pyrimidin-6-yl]quinoline | | | |
| 485 | N-{4-[1-(1-benzylpiperidin-4-yl)-4-morpholin-4-yl-1H- | 498.3 | 1.89 | 2 |
| | pyrazolo[3,4-d]pyrimidin-6-yl]phenyl}formamide | | | |
| 486 | 4-[1-(1-benzylpiperidin-4-yl)-4-morpholin-4-yl-1H- | 471.2 | 1.9 | 2 |
| | pyrazolo[3,4-d]pyrimidin-6-yl]phenol | | | |
| 487 | 4-{4-[6-(1H-indol-5-yl)-4-morpholin-4-yl-1H- | | | 7a |
| | pyrazolo[3,4-d]pyrimidin-1-yl]piperidin-1-yl}-N,N- | | | |
| | dimethyl-4-oxobut-2-en-1-amine | | | |
| 488 | 6-(1H-indol-5-yl)-1-(1-isopropylpiperidin-4-yl)-4- | 446.3 | 1.89 | 16 |
| | morpholin-4-yl-1H-pyrazolo[3,4-d]pyrimidine | | | |
| 489 | 6-(1H-indol-5-yl)-4-morpholin-4-yl-1-[1-(2- | 508.3 | 2.01 | 16 |
| | phenylethyl)piperidin-4-yl]-1H-pyrazolo[3,4-d]pyrimidine | | | |
| 490 | 6-(1H-indol-5-yl)-4-morpholin-4-yl-1-[1-(1- | 508.3 | 2.03 | 16 |
| | phenylethyl)piperidin-4-yl]-1H-pyrazolo[3,4-d]pyrimidine | | | |
| 491 | 6-(1H-indol-5-yl)-1-[1-(2-methoxyethyl)piperidin-4-yl]-4- | 462.3 | 1.87 | 16 |
| | morpholin-4-yl-1H-pyrazolo[3,4-d]pyrimidine | | | |
| 492 | 6-(1H-indol-5-yl)-4-morpholin-4-yl-1-[1- | 522.3 | 2.31 | 7c |
| | (phenylacetyl)piperidin-4-yl]-1H-pyrazolo[3,4- | | | |
| | d]pyrimidine | | | |
| 493 | phenyl 4-[6-(1H-indol-5-yl)-4-morpholin-4-yl-1H- | 524.2 | 2.42 | 7c |
| | pyrazolo[3,4-d]pyrimidin-1-yl]piperidine-1-carboxylate | | | |
| 494 | methyl 4-[6-(1H-indol-5-yl)-4-morpholin-4-yl-1H- | 462.2 | 2.23 | 7c |
| | pyrazolo[3,4-d]pyrimidin-1-yl]piperidine-1-carboxylate | | | |
| 495 | 2-{4-[6-(1H-indol-5-yl)-4-morpholin-4-yl-1H- | 461.2 | 1.78 | 16 |
| | pyrazolo[3,4-d]pyrimidin-1-yl]piperidin-1-yl}acetamide | | | |
| 496 | 2-{4-[6-(1H-indol-5-yl)-4-morpholin-4-yl-1H- | 448.2 | 1.8 | 16 |
| | pyrazolo[3,4-d]pyrimidin-1-yl]piperidin-1-yl}ethanol | | | |
| 497 | 3-{4-[6-(1H-indol-5-yl)-4-morpholin-4-yl-1H- | 462.3 | 1.81 | 16 |
| | pyrazolo[3,4-d]pyrimidin-1-yl]piperidin-1-yl}propan-1-ol | | | |
| 498 | 1-{4-[1-(1-benzylpiperidin-4-yl)-4-morpholin-4-yl-1H- | 513.3 | 1.81 | 8 |
| | pyrazolo[3,4-d]pyrimidin-6-yl]phenyl}urea | | | |
| 499 | 1-{4-[1-(1-benzylpiperidin-4-yl)-4-morpholin-4-yl-1H- | 541.3 | 1.91 | 8 |
| | pyrazolo[3,4-d]pyrimidin-6-yl]phenyl}-3-ethylurea | | | |
| 500 | 1-{4-[1-(1-benzylpiperidin-4-yl)-4-morpholin-4-yl-1H- | 555.3 | 1.97 | 8 |
| | pyrazolo[3,4-d]pyrimidin-6-yl]phenyl}-3-propylurea | | | |
| 501 | propyl {4-[1-(1-benzylpiperidin-4-yl)-4-morpholin-4-yl- | 556.3 | 2.13 | 8 |
| | 1H-pyrazolo[3,4-d]pyrimidin-6-yl]phenyl}carbamate | | | |
| 502 | 1-{4-[1-(1-benzylpiperidin-4-yl)-4-morpholin-4-yl-1H- | 555.3 | 1.97 | 8 |
| | pyrazolo[3,4-d]pyrimidin-6-yl]phenyl}-3-isopropylurea | | | |
| 503 | 1-{4-[1-(1-benzylpiperidin-4-yl)-4-morpholin-4-yl-1H- | 589.3 | 2.1 | 8 |
| | pyrazolo[3,4-d]pyrimidin-6-yl]phenyl}-3-phenylurea | | | |
| 504 | 1-benzyl-3-{4-[1-(1-benzylpiperidin-4-yl)-4-morpholin-4- | 603.3 | 2.06 | 8 |
| | yl-1H-pyrazolo[3,4-d]pyrimidin-6-yl]phenyl}urea | | | |
| 505 | 1-{4-[1-(1-benzylpiperidin-4-yl)-4-morpholin-4-yl-1H- | 617.3 | 2.11 | 8 |
| | pyrazolo[3,4-d]pyrimidin-6-yl]phenyl}-3-(2- | | | |
| | phenylethyl)urea | | | |
| 506 | 1-{4-[1-(1-benzylpiperidin-4-yl)-4-morpholin-4-yl-1H- | 631.3 | 2.16 | 8 |
| | pyrazolo[3,4-d]pyrimidin-6-yl]phenyl}-3-(3- | | | |
| | phenylpropyl)urea | | | |
| 507 | 1-{4-[1-(1-benzylpiperidin-4-yl)-4-morpholin-4-yl-1H- | 590.3 | 1.81 | 8 |
| | pyrazolo[3,4-d]pyrimidin-6-yl]phenyl}-3-pyridin-3-ylurea | | | |
| 508 | 1-{4-[1-(1-benzylpiperidin-4-yl)-4-morpholin-4-yl-1H- | 567.3 | 1.99 | 8 |
| | pyrazolo[3,4-d]pyrimidin-6-yl]phenyl}-3- | | | |
| | (cyclopropylmethyl)urea | | | |
| 509 | 1-allyl-3-{4-[1-(1-benzylpiperidin-4-yl)-4-morpholin-4- | 553.3 | 1.95 | 8 |
| | yl-1H-pyrazolo[3,4-d]pyrimidin-6-yl]phenyl}urea | | | |
| 510 | 1-{4-[1-(1-benzylpiperidin-4-yl)-4-morpholin-4-yl-1H- | 557.3 | 1.79 | 8 |
| | pyrazolo[3,4-d]pyrimidin-6-yl]phenyl}-3-(2- | | | |
| | hydroxyethyl)urea | | | |
| 511 | 1-{4-[1-(1-benzylpiperidin-4-yl)-4-morpholin-4-yl-1H- | 571.3 | 1.88 | 8 |
| | pyrazolo[3,4-d]pyrimidin-6-yl]phenyl}-3-(2- | | | |
| | methoxyethyl)urea | | | |
| 512 | 1-(2-aminoethyl)-3-{4-[1-(1-benzylpiperidin-4-yl)-4- | 556.3 | 1.62 | 8 |
| | morpholin-4-yl-1H-pyrazolo[3,4-d]pyrimidin-6- | | | |
| | yl]phenyl}urea | | | |
| 513 | 1-{4-[1-(1-benzylpiperidin-4-yl)-4-morpholin-4-yl-1H- | 584.3 | 1.65 | 8 |
| | pyrazolo[3,4-d]pyrimidin-6-yl]phenyl}-3-[2- | | | |
| | (dimethylamino)ethyl]urea | | | |
| 514 | 1-{4-[1-(1-benzylpiperidin-4-yl)-4-morpholin-4-yl-1H- | 571.3 | 1.81 | 8 |
| | pyrazolo[3,4-d]pyrimidin-6-yl]phenyl}-3-(3- | | | |
| | hydroxypropyl)urea | | | |
| 515 | 1-{4-[1-(1-benzylpiperidin-4-yl)-4-morpholin-4-yl-1H- | 585.3 | 1.9 | 8 |
| | pyrazolo[3,4-d]pyrimidin-6-yl]phenyl}-3-(3- | | | |
| | methoxypropyl)urea | | | |
| 516 | 1-{4-[1-(1-benzylpiperidin-4-yl)-4-morpholin-4-yl-1H- | 598.4 | 1.67 | 8 |
| | pyrazolo[3,4-d]pyrimidin-6-yl]phenyl}-3-[3- | | | |
| | (dimethylamino)propyl]urea | | | |
| 517 | 1-{4-[1-(1-benzylpiperidin-4-yl)-4-morpholin-4-yl-1H- | 610.4 | 1.68 | 8 |
| | pyrazolo[3,4-d]pyrimidin-6-yl]phenyl}-3-(1- | | | |
| | methylpiperidin-4-yl)urea | | | |
| 518 | 4-[6-(1H-indol-5-yl)-4-morpholin-4-yl-1H-pyrazolo[3,4- | 432.2 | 2.07 | 3 |
| | d]pyrimidin-1-yl]piperidine-1-carbaldehyde | | | |
| 519 | 3-methoxy-N-{4-[4-morpholin-4-yl-1-(tetrahydro-2H- | 515.2 | 2.39 | 43 |
| | pyran-2-yl)-1H-pyrazolo[3,4-d]pyrimidin-6- | | | |
| | yl]phenyl}benzamide | | | |
| 520 | methyl {4-[4-morpholin-4-yl-1-(tetrahydro-2H-pyran-2- | 439.2 | 2.2 | 43 |
| | yl)-1H-pyrazolo[3,4-d]pyrimidin-6-yl]phenyl}carbamate | | | |
| 521 | N²,N²-dimethyl-N-{4-[4-morpholin-4-yl-1-(tetrahydro- | 466.2 | 1.8 | 43 |
| | 2H-pyran-2-yl)-1H-pyrazolo[3,4-d]pyrimidin-6- | | | |
| | yl]phenyl}glycinamide | | | |
| 522 | 2-[4-(dimethylamino)phenyl]-N-{4-[4-morpholin-4-yl-1- | 542.3 | 2.02 | 43 |
| | (tetrahydro-2H-pyran-2-yl)-1H-pyrazolo[3,4-d]pyrimidin- | | | |
| | 6-yl]phenyl}acetamide | | | |
| 523 | 3-methoxy-N-[4-(4-morpholin-4-yl-1H-pyrazolo[3,4- | 431.2 | 2.05 | 43 |
| | d]pyrimidin-6-yl)phenyl]benzamide | | | |
| 524 | N-[4-(4-morpholin-4-yl-1H-pyrazolo[3,4-d]pyrimidin-6- | 402.2 | 1.82 | 43 |
| | yl)phenyl]nicotinamide | | | |
| 525 | methyl [4-(4-morpholin-4-yl-1H-pyrazolo[3,4- | 355.1 | 1.78 | 43 |
| | d]pyrimidin-6-yl)phenyl]carbamate | | | |
| 526 | N-[4-(4-morpholin-4-yl-1H-pyrazolo[3,4-d]pyrimidin-6- | 351.1 | 1.78 | 43 |
| | yl)phenyl]acrylamide | | | |
| 527 | N²,N²-dimethyl-N-[4-(4-morpholin-4-yl-1H-pyrazolo[3,4- | | | |
| | d]pyrimidin-6-yl)phenyl]glycinamide | | | |
| 528 | N-[4-(4-morpholin-4-yl-1H-pyrazolo[3,4-d]pyrimidin-6- | 354.2 | 1.46 | 43 |
| | yl)phenyl]glycinamide | | | |
| 529 | N-[4-(4-morpholin-4-yl-1H-pyrazolo[3,4-d]pyrimidin-6- | 368.2 | 1.49 | 43 |
| | yl)phenyl]-b-alaninamide | | | |
| 530 | 1-methyl-N-[4-(4-morpholin-4-yl-1H-pyrazolo[3,4- | 422.2 | 1.59 | 43 |
| | d]pyrimidin-6-yl)phenyl]piperidine-4-carboxamide | | | |
| 531 | 4-(4-morpholin-4-yl-1H-pyrazolo[3,4-d]pyrimidin-6- | 297.1 | 1.56 | 43 |
| | yl)aniline | | | |
| 532 | 1-{4-[1-(1-benzylpiperidin-4-yl)-4-morpholin-4-yl-1H- | 543.3 | 1.94 | 8 |
| | pyrazolo[3,4-d]pyrimidin-6-yl]phenyl}-3-methoxyurea | | | |
| 533 | 1-{4-[1-(1-benzylpiperidin-4-yl)-4-morpholin-4-yl-1H- | 557.3 | 2 | 8 |
| | pyrazolo[3,4-d]pyrimidin-6-yl]phenyl}-3-ethoxyurea | | | |
| 534 | 1-{4-[1-(1-benzylpiperidin-4-yl)-4-morpholin-4-yl-1H- | 559.3 | 1.94 | 8 |
| | pyrazolo[3,4-d]pyrimidin-6-yl]phenyl}-3-(2- | | | |
| | fluoroethyl)urea | | | |
| 535 | 1-{4-[1-(1-benzylpiperidin-4-yl)-4-morpholin-4-yl-1H- | 595.3 | 2.02 | 8 |
| | pyrazolo[3,4-d]pyrimidin-6-yl]phenyl}-3-(2,2,2- | | | |
| | trifluoroethyl)urea | | | |
| 536 | N-{4-[1-(1-benzylpiperidin-4-yl)-4-morpholin-4-yl-1H- | 556.3 | 1.99 | 8 |
| | pyrazolo[3,4-d]pyrimidin-6-yl]phenyl}-2,2- | | | |
| | dimethylhydrazinecarboxamide | | | |
| 537 | 1-{4-[1-(1-benzylpiperidin-4-yl)-4-morpholin-4-yl-1H- | 582.3 | 2.04 | 8 |
| | pyrazolo[3,4-d]pyrimidin-6-yl]phenyl}-3-pyrrolidin-1- | | | |
| | ylurea | | | |
| 538 | N-[4-(4-morpholin-4-yl-1-phenyl-1H-pyrazolo[3,4- | 431.2 | 2.18 | 30 |
| | d]pyrimidin-6-yl)phenyl]hydrazinecarboxamide | | | |
| 539 | 1-hydroxy-3-[4-(4-morpholin-4-yl-1-phenyl-1H- | 432.2 | 2.19 | 23 |
| | pyrazolo[3,4-d]pyrimidin-6-yl)phenyl]urea | | | |
| 540 | 1-(2-fluoroethyl)-3-[4-(4-morpholin-4-yl-1-phenyl-1H- | 462.2 | 2.31 | 23 |
| | pyrazolo[3,4-d]pyrimidin-6-yl)phenyl]urea | | | |
| 541 | 1-methoxy-3-[4-(4-morpholin-4-yl-1-phenyl-1H- | 446.2 | 2.31 | 23 |
| | pyrazolo[3,4-d]pyrimidin-6-yl)phenyl]urea | | | |
| 542 | 1-(allyloxy)-3-[4-(4-morpholin-4-yl-1-phenyl-1H- | 472.2 | 2.39 | 23 |
| | pyrazolo[3,4-d]pyrimidin-6-yl)phenyl]urea | | | |
| 543 | 1-methyl-3-[4-(4-morpholin-4-yl-1-phenyl-1H- | 430.2 | 2.29 | 23 |
| | pyrazolo[3,4-d]pyrimidin-6-yl)phenyl]urea | | | |
| 544 | N-{4-[1-(1-benzylpiperidin-4-yl)-4-morpholin-4-yl-1H- | 566.2 | 2.17 | 31 |
| | pyrazolo[3,4-d]pyrimidin-6-yl]phenyl}-2,2,2- | | | |
| | trifluoroacetamide | | | |
| 545 | 1-{4-[1-(1-benzylpiperidin-4-yl)-4-morpholin-4-yl-1H- | 570.3 | 1.66 | 32 |
| | pyrazolo[3,4-d]pyrimidin-6-yl]phenyl}-3-[2- | | | |
| | (methylamino)ethyl]urea | | | |
| 546 | 1-(4-{4-morpholin-4-yl-1-[1-(pyridin-3- | 528.2 | 2 | 23 (step |
| | ylcarbonyl)piperidin-4-yl]-1H-pyrazolo[3,4-d]pyrimidin- | | | 2) |
| | 6-yl}phenyl)urea | | | |
| 547 | 1-(4-{4-morpholin-4-yl-1-[1-(pyridin-3- | 605.3 | 1.98 | 23 (step |
| | ylcarbonyl)piperidin-4-yl]-1H-pyrazolo[3,4-d]pyrimidin- | | | 2) |
| | 6-yl}phenyl)-3-pyridin-3-ylurea | | | |
| 548 | 1-(2-fluoroethyl)-3-(4-{4-morpholin-4-yl-1-[1-(pyridin-3- | 574.3 | 2.14 | 23 (step |
| | ylcarbonyl)piperidin-4-yl]-1H-pyrazolo[3,4-d]pyrimidin- | | | 2) |
| | 6-yl}phenyl)urea | | | |
| 549 | 1-(2-hydroxyethyl)-3-(4-{4-morpholin-4-yl-1-[1-(pyridin- | 572.3 | 1.98 | 23 (step |
| | 3-ylcarbonyl)piperidin-4-yl]-1H-pyrazolo[3,4- | | | 2) |
| | d]pyrimidin-6-yl}phenyl)urea | | | |
| 550 | 1-hydroxy-3-(4-{4-morpholin-4-yl-1-[1-(pyridin-3- | 544.2 | 1.96 | 23 (step |
| | ylcarbonyl)piperidin-4-yl]-1H-pyrazolo[3,4-d]pyrimidin- | | | 2) |
| | 6-yl}phenyl)urea | | | |
| 551 | N-(4-{4-morpholin-4-yl-1-[1-(pyridin-3- | 543.3 | 1.95 | 23 (step |
| | ylcarbonyl)piperidin-4-yl]-1H-pyrazolo[3,4-d]pyrimidin- | | | 2) |
| | 6-yl}phenyl)hydrazinecarboxamide | | | |
| 552 | 1-ethyl-3-(4-{4-morpholin-4-yl-1-[1-(pyridin-3- | 556.3 | 2.16 | 23 (step |
| | ylcarbonyl)piperidin-4-yl]-1H-pyrazolo[3,4-d]pyrimidin- | | | 2) |
| | 6-yl}phenyl)urea | | | |
| 553 | 1-methoxy-3-(4-{4-morpholin-4-yl-1-[1-(pyridin-3- | 558.3 | 2.13 | 23 (step |
| | ylcarbonyl)piperidin-4-yl]-1H-pyrazolo[3,4-d]pyrimidin- | | | 2) |
| | 6-yl}phenyl)urea | | | |
| 554 | N-{4-[1-(1-benzylpiperidin-4-yl)-4-morpholin-4-yl-1H- | 528.3 | 1.83 | 23 |
| | pyrazolo[3,4-d]pyrimidin-6- | | | |
| | yl]phenyl}hydrazinecarboxamide | | | |
| 555 | 1-{4-[1-(1-benzylpiperidin-4-yl)-4-morpholin-4-yl-1H- | 529.3 | 1.84 | 23 |
| | pyrazolo[3,4-d]pyrimidin-6-yl]phenyl}-3-hydroxyurea | | | |
| 556 | 1-{4-[1-(1-benzylpiperidin-4-yl)-4-morpholin-4-yl-1H- | 553.3 | 2 | 23 |
| | pyrazolo[3,4-d]pyrimidin-6-yl]phenyl}-3-cyclopropylurea | | | |
| 557 | 1-{4-[1-(1-benzylpiperidin-4-yl)-4-morpholin-4-yl-1H- | 551.3 | 1.98 | 23 |
| | pyrazolo[3,4-d]pyrimidin-6-yl]phenyl}-3-prop-2-yn-1- | | | |
| | ylurea | | | |
| 558 | 1-(4-{4-morpholin-4-yl-1-[1-(pyridin-3- | 514.3 | 1.74 | 23 (step |
| | ylmethyl)piperidin-4-yl]-1H-pyrazolo[3,4-d]pyrimidin-6- | | | 2) |
| | yl}phenyl)urea | | | |
| 559 | 1-(4-{4-morpholin-4-yl-1-[1-(pyridin-3- | 591.3 | 1.71 | 23 (step |
| | ylmethyl)piperidin-4-yl]-1H-pyrazolo[3,4-d]pyrimidin-6- | | | 2) |
| | yl}phenyl)-3-pyridin-3-ylurea | | | |
| 560 | 1-(2-fluoroethyl)-3-(4-{4-morpholin-4-yl-1-[1-(pyridin-3- | 560.3 | 1.83 | 23 (step |
| | ylmethyl)piperidin-4-yl]-1H-pyrazolo[3,4-d]pyrimidin-6- | | | 2) |
| | yl}phenyl)urea | | | |
| 561 | 1-(2-hydroxyethyl)-3-(4-{4-morpholin-4-yl-1-[1-(pyridin- | 558.3 | 1.72 | 23 (step |
| | 3-ylmethyl)piperidin-4-yl]-1H-pyrazolo[3,4-d]pyrimidin- | | | 2) |
| | 6-yl}phenyl)urea | | | |
| 562 | 1-hydroxy-3-(4-{4-morpholin-4-yl-1-[1-(pyridin-3- | 530.3 | 1.7 | 23 (step |
| | ylmethyl)piperidin-4-yl]-1H-pyrazolo[3,4-d]pyrimidin-6- | | | 2) |
| | yl}phenyl)urea | | | |
| 563 | 1-ethyl-3-(4-{4-morpholin-4-yl-1-[1-(pyridin-3- | 542.3 | 1.85 | 23 (step |
| | ylmethyl)piperidin-4-yl]-1H-pyrazolo[3,4-d]pyrimidin-6- | | | 2) |
| | yl}phenyl)urea | | | |
| 564 | 1-methoxy-3-(4-{4-morpholin-4-yl-1-[1-(pyridin-3- | 544.3 | 1.81 | 23 (step |
| | ylmethyl)piperidin-4-yl]-1H-pyrazolo[3,4-d]pyrimidin-6- | | | 2) |
| | yl}phenyl)urea | | | |
| 565 | 4-[1-(1,4-dioxaspiro[4.5]dec-8-yl)-4-morpholin-4-yl-1H- | 437.2 | 2.14 | 4, 23 |
| | pyrazolo[3,4-d]pyrimidin-6-yl]aniline | | | |
| 566 | 1-{4-[1-(1,4-dioxaspiro[4.5]dec-8-yl)-4-morpholin-4-yl- | 494.2 | 2.19 | 4, 23 |
| | 1H-pyrazolo[3,4-d]pyrimidin-6-yl]phenyl}-3-methylurea | | | |
| 567 | 1-methyl-3-{4-[4-morpholin-4-yl-1-(4-oxocyclohexyl)- | 450.2 | 2.06 | 4, 23, 9 |
| | 1H-pyrazolo[3,4-d]pyrimidin-6-yl]phenyl}urea | | | |
| 568 | 1-{4-[1-(4-hydroxycyclohexyl)-4-morpholin-4-yl-1H- | 452.2 | 2 | 4, 23, 9 |
| | pyrazolo[3,4-d]pyrimidin-6-yl]phenyl}-3-methylurea | | | |
| 569 | 1-{4-[1-(1-benzylpiperidin-4-yl)-4-morpholin-4-yl-1H- | 543.3 | 1.95 | 15 |
| | pyrazolo[3,4-d]pyrimidin-6-yl]phenyl}-3-methylthiourea | | | |
| 570 | 1-{4-[1-(1-benzylpiperidin-4-yl)-4-morpholin-4-yl-1H- | 579.3 | 1.77 | 23 |
| | pyrazolo[3,4-d]pyrimidin-6-yl]phenyl}-3-(1H-imidazol-2- | | | |
| | yl)urea | | | |
| 571 | 5-[1-(1-benzylpiperidin-4-yl)-4-morpholin-4-yl-1H- | 511.2 | 1.84 | 33 |
| | pyrazolo[3,4-d]pyrimidin-6-yl]-1,3-dihydro-2H- | | | |
| | benzimidazol-2-one | | | |
| 572 | 1-methyl-3-[4-(4-morpholin-4-yl-1-{1-[(1-oxidopyridin- | 558.3 | 1.98 | 23, 7c |
| | 3-yl)carbonyl]piperidin-4-yl}-1H-pyrazolo[3,4- | | | |
| | d]pyrimidin-6-yl)phenyl]urea | | | |
| 573 | 1-methyl-3-[4-(1-{1-[(2-methylpyridin-3- | 556.3 | 1.97 | 23, 7c |
| | yl)carbonyl]piperidin-4-yl}-4-morpholin-4-yl-1H- | | | |
| | pyrazolo[3,4-d]pyrimidin-6-yl)phenyl]urea | | | |
| 574 | 1-[4-(1-{1-[(6-methoxypyridin-3-yl)methyl]piperidin-4- | 558.3 | 1.86 | 23, 7a |
| | yl}-4-morpholin-4-yl-1H-pyrazolo[3,4-d]pyrimidin-6- | | | |
| | yl)phenyl]-3-methylurea | | | |
| 575 | 1-methyl-3-(4-{4-morpholin-4-yl-1-[1-(pyridin-2- | 528.3 | 1.84 | 23, 7a |
| | ylmethyl)piperidin-4-yl]-1H-pyrazolo[3,4-d]pyrimidin-6- | | | |
| | yl}phenyl)urea | | | |
| 576 | 2-[4-(6-{4-[(methylcarbamoyl)amino]phenyl}-4- | 494.3 | 1.69 | 23, 16 |
| | morpholin-4-yl-1H-pyrazolo[3,4-d]pyrimidin-1- | | | |
| | yl)piperidin-1-yl]acetamide | | | |
| 577 | 1-methyl-3-(4-{4-morpholin-4-yl-1-[1-(2-oxo-2- | 555.3 | 1.91 | 23, 16 |
| | phenylethyl)piperidin-4-yl]-1H-pyrazolo[3,4-d]pyrimidin- | | | |
| | 6-yl}phenyl)urea | | | |
| 578 | 1-methyl-3-[4-(1-{1-[(4-methylpiperazin-1- | 563.3 | 1.8 | 23, 10 |
| | yl)carbonyl]piperidin-4-yl}-4-morpholin-4-yl-1H- | | | |
| | pyrazolo[3,4-d]pyrimidin-6-yl)phenyl]urea | | | |
| 579 | 4-(6-{4-[(methylcarbamoyl)amino]phenyl}-4-morpholin- | 557.3 | 1.85 | 23, 10 |
| | 4-yl-1H-pyrazolo[3,4-d]pyrimidin-1-yl)-N-pyridin-3- | | | |
| | ylpiperidine-1-carboxamide | | | |
| 580 | 1-{4-[1-(1-benzoylpiperidin-4-yl)-4-morpholin-4-yl-1H- | 541.3 | 2.23 | 23, 7c |
| | pyrazolo[3,4-d]pyrimidin-6-yl]phenyl}-3-methylurea | | | |
| 581 | 1-{4-[1-(1-benzoylpiperidin-4-yl)-4-morpholin-4-yl-1H- | 541.26779 | | 22 |
| | pyrazolo[3,4-d]pyrimidin-6-yl]phenyl}-3-methylurea | | | |
| 582 | tert-butyl 4-(6-{4-[(methylcarbamoyl)amino]phenyl}-4- | 537.3 | 2.37 | 23, 7c |
| | morpholin-4-yl-1H-pyrazolo[3,4-d]pyrimidin-1- | | | |
| | yl)piperidine-1-carboxylate | | | |
| 583 | tert-butyl 4-(6-{4-[(methylcarbamoyl)amino]phenyl}-4- | 537.29392 | | 7c |
| | morpholin-4-yl-1H-pyrazolo[3,4-d]pyrimidin-1- | | | |
| | yl)piperidine-1-carboxylate | | | |
| 584 | 1-methyl-3-[4-(4-morpholin-4-yl-1-piperidin-4-yl-1H- | 437.2 | 1.69 | 23, 7c, |
| | pyrazolo[3,4-d]pyrimidin-6-yl)phenyl]urea | | | 22 |
| 585 | 1-methyl-3-[4-(4-morpholin-4-yl-1-piperidin-4-yl-1H- | 437.24211 | | 41 |
| | pyrazolo[3,4-d]pyrimidin-6-yl)phenyl]urea | | | |
| 586 | 1-(4-{4-morpholin-4-yl-1-[1-(pyridin-3- | 590.3 | 2.07 | 23 (step |
| | ylmethyl)piperidin-4-yl]-1H-pyrazolo[3,4-d]pyrimidin-6- | | | 2) |
| | yl}phenyl)-3-phenylurea | | | |
| 587 | 1-(4-{4-morpholin-4-yl-1-[1-(pyridin-3- | 591.3 | 1.7 | 23 (step |
| | ylmethyl)piperidin-4-yl]-1H-pyrazolo[3,4-d]pyrimidin-6- | | | 2) |
| | yl}phenyl)-3-pyridin-4-ylurea | | | |
| 588 | 1-(4-{4-morpholin-4-yl-1-[1-(pyridin-3- | 591.3 | 2.1 | 23 (step |
| | ylmethyl)piperidin-4-yl]-1H-pyrazolo[3,4-d]pyrimidin-6- | | | 2) |
| | yl}phenyl)-3-pyridin-2-ylurea | | | |
| 589 | 1-[2-(methylamino)ethyl]-3-(4-{4-morpholin-4-yl-1-[1- | 571.3 | 1.59 | 23 (step |
| | (pyridin-3-ylmethyl)piperidin-4-yl]-1H-pyrazolo[3,4- | | | 2) |
| | d]pyrimidin-6-yl}phenyl)urea | | | |
| 590 | 1-(4-{4-morpholin-4-yl-1-[1-(pyridin-3- | 604.3 | 2.37 | 23 (step |
| | ylcarbonyl)piperidin-4-yl]-1H-pyrazolo[3,4-d]pyrimidin- | | | 2) |
| | 6-yl}phenyl)-3-phenylurea | | | |
| 591 | 1-(4-{4-morpholin-4-yl-1-[1-(pyridin-3- | 605.3 | 1.99 | 23 (step |
| | ylcarbonyl)piperidin-4-yl]-1H-pyrazolo[3,4-d]pyrimidin- | | | 2) |
| | 6-yl}phenyl)-3-pyridin-4-ylurea | | | |
| 592 | 1-(4-{4-morpholin-4-yl-1-[1-(pyridin-3- | 605.3 | 2.44 | 23 (step |
| | ylcarbonyl)piperidin-4-yl]-1H-pyrazolo[3,4-d]pyrimidin- | | | 2) |
| | 6-yl}phenyl)-3-pyridin-2-ylurea | | | |
| 593 | 1-[2-(methylamino)ethyl]-3-(4-{4-morpholin-4-yl-1-[1- | 585.3 | 1.81 | 23 (step |
| | (pyridin-3-ylcarbonyl)piperidin-4-yl]-1H-pyrazolo[3,4- | | | 2) |
| | d]pyrimidin-6-yl}phenyl)urea | | | |
| 594 | 4-[1-(1-benzylpiperidin-4-yl)-4-morpholin-4-yl-1H- | 488.2 | 1.98 | 34 |
| | pyrazolo[3,4-d]pyrimidin-6-yl]-2-fluoroaniline | | | |
| 595 | 1-{4-[1-(1-benzylpiperidin-4-yl)-4-morpholin-4-yl-1H- | 545.3 | 1.93 | 34 |
| | pyrazolo[3,4-d]pyrimidin-6-yl]-2-fluorophenyl}-3- | | | |
| | methylurea | | | |
| 596 | 1-{4-[1-(1-benzylpiperidin-4-yl)-4-morpholin-4-yl-1H- | 559.3 | 2.03 | 34 |
| | pyrazolo[3,4-d]pyrimidin-6-yl]-2-fluorophenyl}-3- | | | |
| | ethylurea | | | |
| 597 | 1-(2-fluoro-4-{4-morpholin-4-yl-1-[1-(pyridin-3- | 546.3 | 1.84 | 35 |
| | ylmethyl)piperidin-4-yl]-1H-pyrazolo[3,4-d]pyrimidin-6- | | | |
| | yl}phenyl)-3-methylurea | | | |
| 598 | 1-(2-fluoro-4-{4-morpholin-4-yl-1-[1-(pyridin-3- | 608.3 | 2.14 | 35 |
| | ylmethyl)piperidin-4-yl]-1H-pyrazolo[3,4-d]pyrimidin-6- | | | |
| | yl}phenyl)-3-phenylurea | | | |
| 599 | 1-(2-fluoro-4-{4-morpholin-4-yl-1-[1-(pyridin-3- | 609.3 | 1.74 | 35 |
| | ylmethyl)piperidin-4-yl]-1H-pyrazolo[3,4-d]pyrimidin-6- | | | |
| | yl}phenyl)-3-pyridin-4-ylurea | | | |
| 600 | 1-(4-{1-[1-(2-fluorobenzoyl)piperidin-4-yl]-4-morpholin- | 559.3 | 2.19 | 23, 7c |
| | 4-yl-1H-pyrazolo[3,4-d]pyrimidin-6-yl}phenyl)-3- | | | |
| | methylurea | | | |
| 601 | 1-(4-{1-[1-(2-chlorobenzoyl)piperidin-4-yl]-4-morpholin- | 575.2 | 2.24 | 23, 7c |
| | 4-yl-1H-pyrazolo[3,4-d]pyrimidin-6-yl}phenyl)-3- | | | |
| | methylurea | | | |
| 602 | 1-methyl-3-(4-{1-[1-(2-methylbenzoyl)piperidin-4-yl]-4- | 555.3 | 2.24 | 23, 7c |
| | morpholin-4-yl-1H-pyrazolo[3,4-d]pyrimidin-6- | | | |
| | yl}phenyl)urea | | | |
| 603 | 1-(4-{1-[1-(3-fluorobenzoyl)piperidin-4-yl]-4-morpholin- | 559.3 | 2.2 | 23, 7c |
| | 4-yl-1H-pyrazolo[3,4-d]pyrimidin-6-yl}phenyl)-3- | | | |
| | methylurea | | | |
| 604 | 1-(4-{1-[1-(3-chlorobenzoyl)piperidin-4-yl]-4-morpholin- | 575.2 | 2.28 | 23, 7c |
| | 4-yl-1H-pyrazolo[3,4-d]pyrimidin-6-yl}phenyl)-3- | | | |
| | methylurea | | | |
| 605 | 1-methyl-3-[4-(4-morpholin-4-yl-1-{1-[3- | 609.2 | 2.3 | 23, 7c |
| | (trifluoromethyl)benzoyl]piperidin-4-yl}-1H- | | | |
| | pyrazolo[3,4-d]pyrimidin-6-yl)phenyl]urea | | | |
| 606 | 1-(4-{1-[1-(4-bromobenzoyl)piperidin-4-yl]-4-morpholin- | 619.2 | 2.31 | 23, 7c |
| | 4-yl-1H-pyrazolo[3,4-d]pyrimidin-6-yl}phenyl)-3- | | | |
| | methylurea | | | |
| 607 | 1-(4-{1-[1-(4-fluorobenzoyl)piperidin-4-yl]-4-morpholin- | 559.3 | 2.2 | 23, 7c |
| | 4-yl-1H-pyrazolo[3,4-d]pyrimidin-6-yl}phenyl)-3- | | | |
| | methylurea | | | |
| 608 | 1-(4-{1-[1-(4-chlorobenzoyl)piperidin-4-yl]-4-morpholin- | 575.2 | 2.28 | 23, 7c |
| | 4-yl-1H-pyrazolo[3,4-d]pyrimidin-6-yl}phenyl)-3- | | | |
| | methylurea | | | |
| 609 | 1-(4-{1-[1-(4-methoxybenzoyl)piperidin-4-yl]-4- | 571.3 | 2.2 | 23, 7c |
| | morpholin-4-yl-1H-pyrazolo[3,4-d]pyrimidin-6- | | | |
| | yl}phenyl)-3-methylurea | | | |
| 610 | 1-(4-{1-[1-(3-methoxybenzoyl)piperidin-4-yl]-4- | 571.3 | 2.2 | 23, 7c |
| | morpholin-4-yl-1H-pyrazolo[3,4-d]pyrimidin-6- | | | |
| | yl}phenyl)-3-methylurea | | | |
| 611 | 1-(4-{1-[1-(2-methoxybenzoyl)piperidin-4-yl]-4- | 571.3 | 2.19 | 23, 7c |
| | morpholin-4-yl-1H-pyrazolo[3,4-d]pyrimidin-6- | | | |
| | yl}phenyl)-3-methylurea | | | |
| 612 | 1-methyl-3-(4-{1-[1-(3-methylbenzoyl)piperidin-4-yl]-4- | 555.3 | 2.27 | 23, 7c |
| | morpholin-4-yl-1H-pyrazolo[3,4-d]pyrimidin-6- | | | |
| | yl}phenyl)urea | | | |
| 613 | 1-methyl-3-(4-{1-[1-(4-methylbenzoyl)piperidin-4-yl]-4- | 555.3 | 2.26 | 23, 7c |
| | morpholin-4-yl-1H-pyrazolo[3,4-d]pyrimidin-6- | | | |
| | yl}phenyl)urea | | | |
| 614 | 1-(4-{1-[1-(4-cyanobenzoyl)piperidin-4-yl]-4-morpholin- | | | 23, 7c |
| | 4-yl-1H-pyrazolo[3,4-d]pyrimidin-6-yl}phenyl)-3- | | | |
| | methylurea | | | |
| 615 | 2-{4-[1-(1-benzylpiperidin-4-yl)-4-morpholin-4-yl-1H- | 512.3 | 1.83 | 17 |
| | pyrazolo[3,4-d]pyrimidin-6-yl]phenyl}acetamide | | | |
| 616 | 2-{4-[1-(1-benzylpiperidin-4-yl)-4-morpholin-4-yl-1H- | 526.3 | 1.86 | 17 |
| | pyrazolo[3,4-d]pyrimidin-6-yl]phenyl}-N- | | | |
| | methylacetamide | | | |
| 617 | 1-ethyl-3-(2-fluoro-4-{4-morpholin-4-yl-1-[1-(pyridin-3- | 560.3 | 1.9 | 35 |
| | ylmethyl)piperidin-4-yl]-1H-pyrazolo[3,4-d]pyrimidin-6- | | | |
| | yl}phenyl)urea | | | |
| 618 | 1-(2-fluoro-4-{4-morpholin-4-yl-1-[1-(pyridin-3- | 609.3 | 1.78 | 35 |
| | ylmethyl)piperidin-4-yl]-1H-pyrazolo[3,4-d]pyrimidin-6- | | | |
| | yl}phenyl)-3-pyridin-3-ylurea | | | |
| 619 | 1-(2-fluoroethyl)-3-(2-fluoro-4-{4-morpholin-4-yl-1-[1- | | | 35 |
| | (pyridin-3-ylmethyl)piperidin-4-yl]-1H-pyrazolo[3,4- | | | |
| | d]pyrimidin-6-yl}phenyl)urea | | | |
| 620 | 1-(4-{4-morpholin-4-yl-1-[1-(pyridin-3- | 596.2 | 1.99 | 14 |
| | ylmethyl)piperidin-4-yl]-1H-pyrazolo[3,4-d]pyrimidin-6- | | | |
| | yl}phenyl)-3-(3-thienyl)urea | | | |
| 621 | 1-(2-furylmethyl)-3-(4-{4-morpholin-4-yl-1-[1-(pyridin- | 594.3 | 1.91 | 14 |
| | 3-ylmethyl)piperidin-4-yl]-1H-pyrazolo[3,4-d]pyrimidin- | | | |
| | 6-yl}phenyl)urea | | | |
| 622 | 1-methyl-3-(4-{4-morpholin-4-yl-1-[1-(pyridin-3- | 544.3 | 1.78 | 15 |
| | ylmethyl)piperidin-4-yl]-1H-pyrazolo[3,4-d]pyrimidin-6- | | | |
| | yl}phenyl)thiourea | | | |
| 623 | 1-{4-[1-(1-benzoylpiperidin-4-yl)-4-morpholin-4-yl-1H- | 603.3 | 2.43 | 23 (step |
| | pyrazolo[3,4-d]pyrimidin-6-yl]phenyl}-3-phenylurea | | | 2) |
| 624 | 1-{4-[1-(1-benzoylpiperidin-4-yl)-4-morpholin-4-yl-1H- | 604.3 | 2.09 | 23 (step |
| | pyrazolo[3,4-d]pyrimidin-6-yl]phenyl}-3-pyridin-3-ylurea | | | 2) |
| 625 | 1-{4-[1-(1-benzoylpiperidin-4-yl)-4-morpholin-4-yl-1H- | 573.5 | 2.19 | 23 (step |
| | pyrazolo[3,4-d]pyrimidin-6-yl]phenyl}-3-(2- | | | 2) |
| | fluoroethyl)urea | | | |
| 626 | 1-{4-[1-(1-benzoylpiperidin-4-yl)-4-morpholin-4-yl-1H- | 555.3 | 2.24 | 23 (step |
| | pyrazolo[3,4-d]pyrimidin-6-yl]phenyl}-3-ethylurea | | | 2) |
| 627 | 1-{4-[1-(1-benzoylpiperidin-4-yl)-4-morpholin-4-yl-1H- | 527.2 | 2.12 | 23 (step |
| | pyrazolo[3,4-d]pyrimidin-6-yl]phenyl}urea | | | 2) |
| 628 | ethyl {4-[1-(1-benzoylpiperidin-4-yl)-4-morpholin-4-yl- | 556.3 | 2.42 | 23 (step |
| | 1H-pyrazolo[3,4-d]pyrimidin-6-yl]phenyl}carbamate | | | 2) |
| 629 | 1-{4-[1-(1-benzoylpiperidin-4-yl)-4-morpholin-4-yl-1H- | 604.3 | 2.06 | 23 (step |
| | pyrazolo[3,4-d]pyrimidin-6-yl]phenyl}-3-pyridin-4-ylurea | | | 2) |
| 630 | 1-{4-[1-(1-benzoylpiperidin-4-yl)-4-morpholin-4-yl-1H- | 571.3 | 2.1 | 23 (step |
| | pyrazolo[3,4-d]pyrimidin-6-yl]phenyl}-3-(2- | | | 2) |
| | hydroxyethyl)urea | | | |
| 631 | 1-{4-[1-(1-benzoylpiperidin-4-yl)-4-morpholin-4-yl-1H- | 584.3 | 1.93 | 23 (step |
| | pyrazolo[3,4-d]pyrimidin-6-yl]phenyl}-3-[2- | | | 2) |
| | (methylamino)ethyl]urea | | | |
| 632 | 1-[4-(1-{1-[(6-fluoropyridin-3-yl)methyl]piperidin-4-yl}- | 546.3 | 1.8 | 23, 3 |
| | 4-morpholin-4-yl-1H-pyrazolo[3,4-d]pyrimidin-6- | | | |
| | yl)phenyl]-3-methylurea | | | |
| 633 | 1-[4-(1-{1-[(6-chloropyridin-3-yl)methyl]piperidin-4-yl}- | 562.2 | 1.84 | 23, 3 |
| | 4-morpholin-4-yl-1H-pyrazolo[3,4-d]pyrimidin-6- | | | |
| | yl)phenyl]-3-methylurea | | | |
| 634 | 1-[4-(1-{1-[(6-bromopyridin-3-yl)methyl]piperidin-4-yl}- | 606.2 | 1.85 | 23, 3 |
| | 4-morpholin-4-yl-1H-pyrazolo[3,4-d]pyrimidin-6- | | | |
| | yl)phenyl]-3-methylurea | | | |
| 635 | 1-[4-(1-{1-[(2-chloropyridin-3-yl)methyl]piperidin-4-yl}- | 562.2 | 1.83 | 23, 3 |
| | 4-morpholin-4-yl-1H-pyrazolo[3,4-d]pyrimidin-6- | | | |
| | yl)phenyl]-3-methylurea | | | |
| 636 | 1-[4-(1-{1-[(4-methoxypyridin-3-yl)methyl]piperidin-4- | 558.3 | 1.84 | 23, 3 |
| | yl}-4-morpholin-4-yl-1H-pyrazolo[3,4-d]pyrimidin-6- | | | |
| | yl)phenyl]-3-methylurea | | | |
| 637 | 1-[4-(1-{1-[(5-fluoropyridin-3-yl)methyl]piperidin-4-yl}- | 546.3 | 1.81 | 23, 3 |
| | 4-morpholin-4-yl-1H-pyrazolo[3,4-d]pyrimidin-6- | | | |
| | yl)phenyl]-3-methylurea | | | |
| 638 | 1-[4-(1-{1-[(5-bromopyridin-3-yl)methyl]piperidin-4-yl}- | 606.2 | 1.86 | 23, 3 |
| | 4-morpholin-4-yl-1H-pyrazolo[3,4-d]pyrimidin-6- | | | |
| | yl)phenyl]-3-methylurea | | | |
| 639 | 1-(4-{1-[1-(4-chlorobenzyl)piperidin-4-yl]-4-morpholin- | 561.2 | 1.98 | 23, 3 |
| | 4-yl-1H-pyrazolo[3,4-d]pyrimidin-6-yl}phenyl)-3- | | | |
| | methylurea | | | |
| 640 | 1-(4-{1-[1-(3-chlorobenzyl)piperidin-4-yl]-4-morpholin- | 561.2 | 1.95 | 23, 3 |
| | 4-yl-1H-pyrazolo[3,4-d]pyrimidin-6-yl}phenyl)-3- | | | |
| | methylurea | | | |
| 641 | 1-(4-{1-[1-(2-chlorobenzyl)piperidin-4-yl]-4-morpholin- | 561.2 | 1.96 | 23, 3 |
| | 4-yl-1H-pyrazolo[3,4-d]pyrimidin-6-yl}phenyl)-3- | | | |
| | methylurea | | | |
| 642 | 1-(4-{1-[1-(3-hydroxybenzyl)piperidin-4-yl]-4- | 543.3 | 1.81 | 23, 3 |
| | morpholin-4-yl-1H-pyrazolo[3,4-d]pyrimidin-6- | | | |
| | yl}phenyl)-3-methylurea | | | |
| 643 | 1-(4-{1-[1-(3-hydroxy-4-methoxybenzyl)piperidin-4-yl]- | 573.3 | 1.83 | 23, 3 |
| | 4-morpholin-4-yl-1H-pyrazolo[3,4-d]pyrimidin-6- | | | |
| | yl}phenyl)-3-methylurea | | | |
| 644 | 1-(4-{1-[1-(2-hydroxybenzyl)piperidin-4-yl]-4- | 543.3 | 1.85 | 23, 3 |
| | morpholin-4-yl-1H-pyrazolo[3,4-d]pyrimidin-6- | | | |
| | yl}phenyl)-3-methylurea | | | |
| 645 | 1-(4-{1-[1-(3-methoxybenzyl)piperidin-4-yl]-4- | 557.3 | 1.9 | 23, 3 |
| | morpholin-4-yl-1H-pyrazolo[3,4-d]pyrimidin-6- | | | |
| | yl}phenyl)-3-methylurea | | | |
| 646 | 1-methyl-3-{4-[1-(1-methylpiperidin-4-yl)-4-morpholin- | 451.2 | 1.71 | 23, 3 |
| | 4-yl-1H-pyrazolo[3,4-d]pyrimidin-6-yl]phenyl}urea | | | |
| 647 | 1-(4-{1-[1-(2-furylmethyl)piperidin-4-yl]-4-morpholin-4- | 517.3 | 1.81 | 23, 3 |
| | yl-1H-pyrazolo[3,4-d]pyrimidin-6-yl}phenyl)-3- | | | |
| | methylurea | | | |
| 648 | 1-(4-{4-morpholin-4-yl-1-[1-(pyridin-3- | 596.2 | 1.97 | 14 |
| | ylmethyl)piperidin-4-yl]-1H-pyrazolo[3,4-d]pyrimidin-6- | | | |
| | yl}phenyl)-3-(2-thienyl)urea | | | |
| 649 | 1-cyclopropyl-3-(4-{4-morpholin-4-yl-1-[1-(pyridin-3- | 554.3 | 1.83 | 23 |
| | ylmethyl)piperidin-4-yl]-1H-pyrazolo[3,4-d]pyrimidin-6- | | | |
| | yl}phenyl)urea | | | |
| 650 | 2-cyano-1-(4-{4-morpholin-4-yl-1-[1-(pyridin-3- | 538.3 | 1.74 | 18 |
| | ylmethyl)piperidin-4-yl]-1H-pyrazolo[3,4-d]pyrimidin-6- | | | |
| | yl}phenyl)guanidine | | | |
| 651 | 2-cyano-1-methyl-3-(4-{4-morpholin-4-yl-1-[1-(pyridin- | 552.3 | 1.78 | 18 |
| | 3-ylmethyl)piperidin-4-yl]-1H-pyrazolo[3,4-d]pyrimidin- | | | |
| | 6-yl}phenyl)guanidine | | | |
| 652 | 2-cyano-1-ethyl-3-(4-{4-morpholin-4-yl-1-[1-(pyridin-3- | 566.3 | 1.81 | 18 |
| | ylmethyl)piperidin-4-yl]-1H-pyrazolo[3,4-d]pyrimidin-6- | | | |
| | yl}phenyl)guanidine | | | |
| 653 | N′-cyano-N-(4-{4-morpholin-4-yl-1-[1-(pyridin-3- | 539.3 | 1.77 | 18 |
| | ylmethyl)piperidin-4-yl]-1H-pyrazolo[3,4-d]pyrimidin-6- | | | |
| | yl}phenyl)carbamimidic acid | | | |
| 654 | methyl N′-cyano-N-(4-{4-morpholin-4-yl-1-[1-(pyridin-3- | 553.3 | 1.86 | 18 |
| | ylmethyl)piperidin-4-yl]-1H-pyrazolo[3,4-d]pyrimidin-6- | | | |
| | yl}phenyl)imidocarbamate | | | |
| 655 | 2-cyano-1-(4-{4-morpholin-4-yl-1-[1-(pyridin-3- | 552.3 | 1.97 | 18 |
| | ylcarbonyl)piperidin-4-yl]-1H-pyrazolo[3,4-d]pyrimidin- | | | |
| | 6-yl}phenyl)guanidine | | | |
| 656 | 2-cyano-1-methyl-3-(4-{4-morpholin-4-yl-1-[1-(pyridin- | 566.3 | 1.98 | 18 |
| | 3-ylcarbonyl)piperidin-4-yl]-1H-pyrazolo[3,4- | | | |
| | d]pyrimidin-6-yl}phenyl)guanidine | | | |
| 657 | 1-(4-{4-morpholin-4-yl-1-[1-(pyridin-3- | 610.2 | 2.23 | 14 |
| | ylcarbonyl)piperidin-4-yl]-1H-pyrazolo[3,4-d]pyrimidin- | | | |
| | 6-yl}phenyl)-3-(2-thienyl)urea | | | |
| 658 | 1-(4-{4-morpholin-4-yl-1-[1-(pyridin-3- | 610.2 | 2.25 | 14 |
| | ylcarbonyl)piperidin-4-yl]-1H-pyrazolo[3,4-d]pyrimidin- | | | |
| | 6-yl}phenyl)-3-(3-thienyl)urea | | | |
| 659 | 1-cyclopropyl-3-(4-{4-morpholin-4-yl-1-[1-(pyridin-3- | 568.3 | 2.07 | 23 |
| | ylcarbonyl)piperidin-4-yl]-1H-pyrazolo[3,4-d]pyrimidin- | | | |
| | 6-yl}phenyl)urea | | | |
| 660 | 6-(2,3-dihydro-1H-indol-5-yl)-4-morpholin-4-yl-1-[1- | 497.3 | 1.64 | 36 |
| | (pyridin-3-ylmethyl)piperidin-4-yl]-1H-pyrazolo[3,4- | | | |
| | d]pyrimidine | | | |
| 661 | 1-{4-[1-(1-isonicotinoylpiperidin-4-yl)-4-morpholin-4-yl- | 542.3 | 2.08 | 23, 7c |
| | 1H-pyrazolo[3,4-d]pyrimidin-6-yl]phenyl}-3-methylurea | | | |
| 662 | 1-methyl-3-(4-{4-morpholin-4-yl-1-[1-(pyridin-2- | 542.3 | 2.18 | 23, 7c |
| | ylcarbonyl)piperidin-4-yl]-1H-pyrazolo[3,4-d]pyrimidin- | | | |
| | 6-yl}phenyl)urea | | | |
| 663 | 1-methyl-3-[4-(1-{1-[(4-methylpyridin-3- | 556.3 | 2.04 | 23, 7c |
| | yl)carbonyl]piperidin-4-yl}-4-morpholin-4-yl-1H- | | | |
| | pyrazolo[3,4-d]pyrimidin-6-yl)phenyl]urea | | | |
| 664 | 1-methyl-3-[4-(1-{1-[(6-methylpyridin-3- | 556.3 | 2.05 | 23, 7c |
| | yl)carbonyl]piperidin-4-yl}-4-morpholin-4-yl-1H- | | | |
| | pyrazolo[3,4-d]pyrimidin-6-yl)phenyl]urea | | | |
| 665 | 1-[4-(1-{1-[(6-fluoropyridin-3-yl)carbonyl]piperidin-4- | 560.2 | 2.2 | 23, 7c |
| | yl}-4-morpholin-4-yl-1H-pyrazolo[3,4-d]pyrimidin-6- | | | |
| | yl)phenyl]-3-methylurea | | | |
| 666 | 1-methyl-3-(4-{4-morpholin-4-yl-1-[1-(pyrazin-2- | 543.3 | 2.11 | 23, 7c |
| | ylcarbonyl)piperidin-4-yl]-1H-pyrazolo[3,4-d]pyrimidin- | | | |
| | 6-yl}phenyl)urea | | | |
| 667 | 1-(4-{1-[1-(3-acetylbenzoyl)piperidin-4-yl]-4-morpholin- | 583.3 | 2.27 | 23, 7c |
| | 4-yl-1H-pyrazolo[3,4-d]pyrimidin-6-yl}phenyl)-3- | | | |
| | methylurea | | | |
| 668 | 1-[4-(1-{1-[(6-chloropyridin-3-yl)carbonyl]piperidin-4- | 576.2 | 2.28 | 23, 7c |
| | yl}-4-morpholin-4-yl-1H-pyrazolo[3,4-d]pyrimidin-6- | | | |
| | yl)phenyl]-3-methylurea | | | |
| 669 | 1-[4-(1-{1-[(2-chloropyridin-3-yl)carbonyl]piperidin-4- | 576.2 | 2.23 | 23, 7c |
| | yl}-4-morpholin-4-yl-1H-pyrazolo[3,4-d]pyrimidin-6- | | | |
| | yl)phenyl]-3-methylurea | | | |
| 670 | 1-methyl-3-(4-{4-morpholin-4-yl-1-[1-(pyridin-4- | 528.3 | 1.72 | 23, 3 |
| | ylmethyl)piperidin-4-yl]-1H-pyrazolo[3,4-d]pyrimidin-6- | | | |
| | yl}phenyl)urea | | | |
| 671 | 1-(4-{1-[1-(4-fluorobenzyl)piperidin-4-yl]-4-morpholin-4- | 545.3 | 1.89 | 23, 3 |
| | yl-1H-pyrazolo[3,4-d]pyrimidin-6-yl}phenyl)-3- | | | |
| | methylurea | | | |
| 672 | 1-(4-{1-[1-(3-fluorobenzyl)piperidin-4-yl]-4-morpholin-4- | 545.3 | 1.89 | 23, 3 |
| | yl-1H-pyrazolo[3,4-d]pyrimidin-6-yl}phenyl)-3- | | | |
| | methylurea | | | |
| 673 | 1-methyl-3-(4-{1-[1-(2-methylbenzyl)piperidin-4-yl]-4- | 541.3 | 1.92 | 23, 3 |
| | morpholin-4-yl-1H-pyrazolo[3,4-d]pyrimidin-6- | | | |
| | yl}phenyl)urea | | | |
| 674 | 1-methyl-3-(4-{1-[1-(3-methylbenzyl)piperidin-4-yl]-4- | 541.3 | 1.94 | 23, 3 |
| | morpholin-4-yl-1H-pyrazolo[3,4-d]pyrimidin-6- | | | |
| | yl}phenyl)urea | | | |
| 675 | 1-methyl-3-(4-{1-[1-(4-methylbenzyl)piperidin-4-yl]-4- | 541.3 | 1.94 | 23, 3 |
| | morpholin-4-yl-1H-pyrazolo[3,4-d]pyrimidin-6- | | | |
| | yl}phenyl)urea | | | |
| 676 | 6-(1H-indazol-5-yl)-4-morpholin-4-yl-1-phenyl-1H- | 398.2 | 2.66 | 2 |
| | pyrazolo[3,4-d]pyrimidine | | | |
| 677 | 5-(4-morpholin-4-yl-1-phenyl-1H-pyrazolo[3,4- | 413.2 | 2.53 | 36 |
| | d]pyrimidin-6-yl)-1,3-dihydro-2H-indol-2-one | | | |
| 678 | 2-{4-morpholin-4-yl-1-[1-(pyridin-3- | 486.2 | 1.69 | 2 |
| | ylcarbonyl)piperidin-4-yl]-1H-pyrazolo[3,4-d]pyrimidin- | | | |
| | 6-yl}pyridin-4-amine | | | |
| 679 | 6-{4-morpholin-4-yl-1-[1-(pyridin-3- | 486.2 | 1.71 | 2 |
| | ylcarbonyl)piperidin-4-yl]-1H-pyrazolo[3,4-d]pyrimidin- | | | |
| | 6-yl}pyridin-3-amine | | | |
| 680 | 6-{4-morpholin-4-yl-1-[1-(pyridin-3- | 486.2 | 1.73 | 2 |
| | ylcarbonyl)piperidin-4-yl]-1H-pyrazolo[3,4-d]pyrimidin- | | | |
| | 6-yl}pyridin-2-amine | | | |
| 681 | 2-(4-morpholin-4-yl-1-phenyl-1H-pyrazolo[3,4- | 374.2 | 1.87 | 2 |
| | d]pyrimidin-6-yl)pyridin-4-amine | | | |
| 682 | 6-(4-morpholin-4-yl-1-phenyl-1H-pyrazolo[3,4- | 374.2 | 1.93 | 2 |
| | d]pyrimidin-6-yl)pyridin-3-amine | | | |
| 683 | 6-(4-morpholin-4-yl-1-phenyl-1H-pyrazolo[3,4- | 374.2 | 1.9 | 2 |
| | d]pyrimidin-6-yl)pyridin-2-amine | | | |
| 684 | methyl [4-(1-{1-[(4-methylpyridin-3- | 557.3 | 2.19 | 23, 7c |
| | yl)carbonyl]piperidin-4-yl}-4-morpholin-4-yl-1H- | | | |
| | pyrazolo[3,4-d]pyrimidin-6-yl)phenyl]carbamate | | | |
| 685 | methyl (4-{4-morpholin-4-yl-1-[1-(pyridin-2- | 543.2 | 2.32 | 23, 7c |
| | ylcarbonyl)piperidin-4-yl]-1H-pyrazolo[3,4-d]pyrimidin- | | | |
| | 6-yl}phenyl)carbamate | | | |
| 686 | methyl {4-[1-(1-benzoylpiperidin-4-yl)-4-morpholin-4-yl- | 542.2 | 2.44 | 23, 7c |
| | 1H-pyrazolo[3,4-d]pyrimidin-6-yl]phenyl}carbamate | | | |
| 687 | methyl (4-{1-[1-(2-fluorobenzoyl)piperidin-4-yl]-4- | 560.2 | 2.44 | 23, 7c |
| | morpholin-4-yl-1H-pyrazolo[3,4-d]pyrimidin-6- | | | |
| | yl}phenyl)carbamate | | | |
| 688 | methyl (4-{1-[1-(2-chlorobenzoyl)piperidin-4-yl]-4- | 576.2 | 2.49 | 23, 7c |
| | morpholin-4-yl-1H-pyrazolo[3,4-d]pyrimidin-6- | | | |
| | yl}phenyl)carbamate | | | |
| 689 | methyl (4-{1-[1-(4-fluorobenzoyl)piperidin-4-yl]-4- | 560.2 | 2.44 | 23, 7c |
| | morpholin-4-yl-1H-pyrazolo[3,4-d]pyrimidin-6- | | | |
| | yl}phenyl)carbamate | | | |
| 690 | methyl (4-{1-[1-(2-methoxybenzoyl)piperidin-4-yl]-4- | 572.3 | 2.44 | 23, 7c |
| | morpholin-4-yl-1H-pyrazolo[3,4-d]pyrimidin-6- | | | |
| | yl}phenyl)carbamate | | | |
| 691 | methyl (4-{1-[1-(4-cyanobenzoyl)piperidin-4-yl]-4- | 567.3 | 2.34 | 23, 7c |
| | morpholin-4-yl-1H-pyrazolo[3,4-d]pyrimidin-6- | | | |
| | yl}phenyl)carbamate | | | |
| 692 | methyl [4-(1-{1-[(4-methylpiperazin-1- | 564.3 | 1.95 | 23, 10 |
| | yl)carbonyl]piperidin-4-yl}-4-morpholin-4-yl-1H- | | | |
| | pyrazolo[3,4-d]pyrimidin-6-yl)phenyl]carbamate | | | |
| 693 | methyl (4-{1-[1-(2,4-difluorobenzoyl)piperidin-4-yl]-4- | 578.2 | 2.46 | 23, 7c |
| | morpholin-4-yl-1H-pyrazolo[3,4-d]pyrimidin-6- | | | |
| | yl}phenyl)carbamate | | | |
| 694 | methyl (4-{1-[1-(4-fluorobenzyl)piperidin-4-yl]-4- | | | 23, 3 |
| | morpholin-4-yl-1H-pyrazolo[3,4-d]pyrimidin-6- | | | |
| | yl}phenyl)carbamate | | | |
| 695 | methyl (4-{1-[1-(4-chlorobenzyl)piperidin-4-yl]-4- | 562.2 | 2.1 | 23, 3 |
| | morpholin-4-yl-1H-pyrazolo[3,4-d]pyrimidin-6- | | | |
| | yl}phenyl)carbamate | | | |
| 696 | methyl [4-(1-{1-[(2-methoxypyridin-3- | 559.3 | 2 | 23, 3 |
| | yl)methyl]piperidin-4-yl}-4-morpholin-4-yl-1H- | | | |
| | pyrazolo[3,4-d]pyrimidin-6-yl)phenyl]carbamate | | | |
| 697 | methyl [4-(1-{1-[(6-fluoropyridin-3-yl)methyl]piperidin- | 543.5 | 2.42 | 23, 3 |
| | 4-yl}-4-morpholin-4-yl-1H-pyrazolo[3,4-d]pyrimidin-6- | | | |
| | yl)phenyl]carbamate | | | |
| 698 | methyl [4-(1-{1-[(6-chloropyridin-3-yl)methyl]piperidin- | 563.2 | 1.97 | 23, 3 |
| | 4-yl}-4-morpholin-4-yl-1H-pyrazolo[3,4-d]pyrimidin-6- | | | |
| | yl)phenyl]carbamate | | | |
| 699 | methyl (4-{1-[1-(2-chlorobenzyl)piperidin-4-yl]-4- | 562.2 | 2.08 | 23, 3 |
| | morpholin-4-yl-1H-pyrazolo[3,4-d]pyrimidin-6- | | | |
| | yl}phenyl)carbamate | | | |
| 700 | methyl (4-{1-[1-(2-amino-2-oxoethyl)piperidin-4-yl]-4- | 495.2 | 1.81 | 23, 16 |
| | morpholin-4-yl-1H-pyrazolo[3,4-d]pyrimidin-6- | | | |
| | yl}phenyl)carbamate | | | |
| 701 | methyl (4-{4-morpholin-4-yl-1-[1-(2-oxo-2- | 556.3 | 2.02 | 23, 16 |
| | phenylethyl)piperidin-4-yl]-1H-pyrazolo[3,4-d]pyrimidin- | | | |
| | 6-yl}phenyl)carbamate | | | |
| 702 | methyl {4-[1-(1-acryloylpiperidin-4-yl)-4-morpholin-4-yl- | 492.2 | 2.28 | 23, 7c |
| | 1H-pyrazolo[3,4-d]pyrimidin-6-yl]phenyl}carbamate | | | |
| 703 | methyl [4-(4-morpholin-4-yl-1-piperidin-4-yl-1H- | 438.2 | 1.82 | 23, 3 |
| | pyrazolo[3,4-d]pyrimidin-6-yl)phenyl]carbamate | | | |
| 704 | 3-fluoro-4-{4-morpholin-4-yl-1-[1-(pyridin-3- | 503.2 | 2.04 | 8 |
| | ylcarbonyl)piperidin-4-yl]-1H-pyrazolo[3,4-d]pyrimidin- | | | |
| | 6-yl}aniline | | | |
| 705 | 1-(3-fluoro-4-{4-morpholin-4-yl-1-[1-(pyridin-3- | 560.2 | 2.04 | 8, 23 |
| | ylcarbonyl)piperidin-4-yl]-1H-pyrazolo[3,4-d]pyrimidin- | | | |
| | 6-yl}phenyl)-3-methylurea | | | |
| 706 | 1-ethyl-3-(3-fluoro-4-{4-morpholin-4-yl-1-[1-(pyridin-3- | 574.3 | 2.11 | 8, 23 |
| | ylcarbonyl)piperidin-4-yl]-1H-pyrazolo[3,4-d]pyrimidin- | | | |
| | 6-yl}phenyl)urea | | | |
| 707 | 1-(2-fluoroethyl)-3-(3-fluoro-4-{4-morpholin-4-yl-1-[1- | 592.3 | 2.09 | 8, 23 |
| | (pyridin-3-ylcarbonyl)piperidin-4-yl]-1H-pyrazolo[3,4- | | | |
| | d]pyrimidin-6-yl}phenyl)urea | | | |
| 708 | 2,5-difluoro-4-{4-morpholin-4-yl-1-[1-(pyridin-3- | 521.2 | 2.18 | 8 |
| | ylcarbonyl)piperidin-4-yl]-1H-pyrazolo[3,4-d]pyrimidin- | | | |
| | 6-yl}aniline | | | |
| 709 | 1-(2,5-difluoro-4-{4-morpholin-4-yl-1-[1-(pyridin-3- | 578.2 | 2.16 | 8, 23 |
| | ylcarbonyl)piperidin-4-yl]-1H-pyrazolo[3,4-d]pyrimidin- | | | |
| | 6-yl}phenyl)-3-methylurea | | | |
| 710 | 1-(2,5-difluoro-4-{4-morpholin-4-yl-1-[1-(pyridin-3- | 592.3 | 2.24 | 8, 23 |
| | ylcarbonyl)piperidin-4-yl]-1H-pyrazolo[3,4-d]pyrimidin- | | | |
| | 6-yl}phenyl)-3-ethylurea | | | |
| 711 | 1-(2,5-difluoro-4-{4-morpholin-4-yl-1-[1-(pyridin-3- | 610.2 | 2.24 | 8, 23 |
| | ylcarbonyl)piperidin-4-yl]-1H-pyrazolo[3,4-d]pyrimidin- | | | |
| | 6-yl}phenyl)-3-(2-fluoroethyl)urea | | | |
| 712 | 1-cyclopropyl-3-(2,5-difluoro-4-{4-morpholin-4-yl-1-[1- | 604.3 | 2.29 | 8, 23 |
| | (pyridin-3-ylcarbonyl)piperidin-4-yl]-1H-pyrazolo[3,4- | | | |
| | d]pyrimidin-6-yl}phenyl)urea | | | |
| 713 | 1-(2,5-difluoro-4-{4-morpholin-4-yl-1-[1-(pyridin-3- | 640.3 | 2.57 | 8, 23 |
| | ylcarbonyl)piperidin-4-yl]-1H-pyrazolo[3,4-d]pyrimidin- | | | |
| | 6-yl}phenyl)-3-phenylurea | | | |
| 714 | 1-methyl-3-(4-{4-morpholin-4-yl-1-[1-(2,2,2- | 519.2 | 2.22 | 48 |
| | trifluoroethyl)piperidin-4-yl]-1H-pyrazolo[3,4- | | | |
| | d]pyrimidin-6-yl}phenyl)urea | | | |
| 715 | 6-(1H-indol-5-yl)-4-morpholin-4-yl-1-[1-(2,2,2- | 486.2 | 2.44 | 47 |
| | trifluoroethyl)piperidin-4-yl]-1H-pyrazolo[3,4- | | | |
| | d]pyrimidine | | | |
| 716 | 1-{4-[1-(1-acetylpiperidin-4-yl)-4-morpholin-4-yl-1H- | 479.2 | 2.07 | 23, 7c |
| | pyrazolo[3,4-d]pyrimidin-6-yl]phenyl}-3-methylurea | | | |
| 717 | N,N-dimethyl-4-(6-{4- | 508.3 | 2.17 | 23, 10 |
| | [(methylcarbamoyl)amino]phenyl}-4-morpholin-4-yl-1H- | | | |
| | pyrazolo[3,4-d]pyrimidin-1-yl)piperidine-1-carboxamide | | | |
| 718 | 1-(4-{1-[1-(methoxyacetyl)piperidin-4-yl]-4-morpholin-4- | 509.3 | 2.05 | 23, 7c |
| | yl-1H-pyrazolo[3,4-d]pyrimidin-6-yl}phenyl)-3- | | | |
| | methylurea | | | |
| 719 | 1-{4-[1-(1-isobutyrylpiperidin-4-yl)-4-morpholin-4-yl- | 507.3 | 2.19 | 23, 7c |
| | 1H-pyrazolo[3,4-d]pyrimidin-6-yl]phenyl}-3-methylurea | | | |
| 720 | methyl 4-(6-{4-[(methylcarbamoyl)amino]phenyl}-4- | 495.2 | 2.18 | 23, 3 |
| | morpholin-4-yl-1H-pyrazolo[3,4-d]pyrimidin-1- | | | |
| | yl)piperidine-1-carboxylate | | | |
| 721 | methyl 4-(6-{4-[(methylcarbamoyl)amino]phenyl}-4- | 495.24607 | | |
| | morpholin-4-yl-1H-pyrazolo[3,4-d]pyrimidin-1- | | | |
| | yl)piperidine-1-carboxylate | | | |
| 722 | N-methyl-4-(6-{4-[(methylcarbamoyl)amino]phenyl}-4- | 494.3 | 2.01 | 23, 3 |
| | morpholin-4-yl-1H-pyrazolo[3,4-d]pyrimidin-1- | | | |
| | yl)piperidine-1-carboxamide | | | |
| 723 | 3-{4-[(2R,6S)-2,6-dimethylmorpholin-4-yl]-1-phenyl-1H- | 402.2 | 2.59 | 51 |
| | pyrazolo[3,4-d]pyrimidin-6-yl}phenol | | | |
| 724 | 1-ethyl-3-(5-{4-morpholin-4-yl-1-[1-(pyridin-3- | 543.3 | 1.87 | 14 |
| | ylmethyl)piperidin-4-yl]-1H-pyrazolo[3,4-d]pyrimidin-6- | | | |
| | yl}pyridin-2-yl)urea | | | |
| 725 | 1-methyl-3-(5-{4-morpholin-4-yl-1-[1-(pyridin-3- | 543.3 | 2.1 | 37 |
| | ylcarbonyl)piperidin-4-yl]-1H-pyrazolo[3,4-d]pyrimidin- | | | |
| | 6-yl}pyridin-2-yl)urea | | | |
| 726 | 3-{4-[(3S)-3-methylmorpholin-4-yl]-1-phenyl-1H- | 388.2 | 2.47 | 52 |
| | pyrazolo[3,4-d]pyrimidin-6-yl}phenol | | | |
| 727 | 4-[6-(3-hydroxyphenyl)-1-phenyl-1H-pyrazolo[3,4- | 388.1 | 2.36 | 53, 54 |
| | d]pyrimidin-4-yl]morpholin-3-one | | | |
| 728 | 3-[4-morpholin-4-yl-1-(2,2,2-trifluoroethyl)-1H- | 380.1 | 2.19 | 55 |
| | pyrazolo[3,4-d]pyrimidin-6-yl]phenol | | | |
| 729 | 1-methyl-3-{4-[4-morpholin-4-yl-1-(2,2,2-trifluoroethyl)- | 436.2 | 2.14 | 56 |
| | 1H-pyrazolo[3,4-d]pyrimidin-6-yl]phenyl}urea | | | |
| 730 | 1-(2-chloro-4-{4-morpholin-4-yl-1-[1-(pyridin-3- | 562.2 | 1.92 | 38 |
| | ylmethyl)piperidin-4-yl]-1H-pyrazolo[3,4-d]pyrimidin-6- | | | |
| | yl}phenyl)-3-methylurea | | | |
| 731 | methyl 4-(6-{4-[(ethylcarbamoyl)amino]phenyl}-4- | 509.3 | 2.21 | 39, 23 |
| | morpholin-4-yl-1H-pyrazolo[3,4-d]pyrimidin-1- | | | |
| | yl)piperidine-1-carboxylate | | | |
| 732 | methyl 4-[6-(4-{[(2- | 527.2 | 2.19 | 39, 23 |
| | fluoroethyl)carbamoyl]amino}phenyl)-4-morpholin-4-yl- | | | |
| | 1H-pyrazolo[3,4-d]pyrimidin-1-yl]piperidine-1- | | | |
| | carboxylate | | | |
| 733 | methyl 4-[6-(4-{[(2- | 525.2 | 2.07 | 39, 23 |
| | hydroxyethyl)carbamoyl]amino}phenyl)-4-morpholin-4- | | | |
| | yl-1H-pyrazolo[3,4-d]pyrimidin-1-yl]piperidine-1- | | | |
| | carboxylate | | | |
| 734 | methyl 4-(6-{4-[(cyclopropylcarbamoyl)amino]phenyl}- | 521.3 | 2.23 | 39, 23 |
| | 4-morpholin-4-yl-1H-pyrazolo[3,4-d]pyrimidin-1- | | | |
| | yl)piperidine-1-carboxylate | | | |
| 735 | methyl 4-(6-{4-[(anilinocarbonyl)amino]phenyl}-4- | 557.3 | 2.43 | 39, 23 |
| | morpholin-4-yl-1H-pyrazolo[3,4-d]pyrimidin-1- | | | |
| | yl)piperidine-1-carboxylate | | | |
| 736 | methyl 4-(4-morpholin-4-yl-6-{4-[(pyridin-2- | 558.3 | 2.44 | 39, 23 |
| | ylcarbamoyl)amino]phenyl}-1H-pyrazolo[3,4- | | | |
| | d]pyrimidin-1-yl)piperidine-1-carboxylate | | | |
| 737 | methyl 4-(4-morpholin-4-yl-6-{4-[(pyridin-3- | 558.3 | 2.05 | 39, 23 |
| | ylcarbamoyl)amino]phenyl}-1H-pyrazolo[3,4- | | | |
| | d]pyrimidin-1-yl)piperidine-1-carboxylate | | | |
| 738 | methyl 4-(4-morpholin-4-yl-6-{4-[(pyridin-4- | 558.3 | 2.01 | 39, 23 |
| | ylcarbamoyl)amino]phenyl}-1H-pyrazolo[3,4- | | | |
| | d]pyrimidin-1-yl)piperidine-1-carboxylate | | | |
| 739 | methyl 4-(6-{4-[(methoxycarbonyl)amino]phenyl}-4- | 496.2 | 2.33 | 39, 23 |
| | morpholin-4-yl-1H-pyrazolo[3,4-d]pyrimidin-1- | | | |
| | yl)piperidine-1-carboxylate | | | |
| 740 | methyl 4-(6-{4-[(methoxycarbonyl)amino]phenyl}-4- | 496.23039 | | |
| | morpholin-4-yl-1H-pyrazolo[3,4-d]pyrimidin-1- | | | |
| | yl)piperidine-1-carboxylate | | | |
| 741 | methyl 4-[6-(4-aminophenyl)-4-morpholin-4-yl-1H- | 438.2 | 2.09 | 39, 23 |
| | pyrazolo[3,4-d]pyrimidin-1-yl]piperidine-1-carboxylate | | | |
| 742 | methyl 4-[6-(4- | 511.2 | 2.2 | 15 |
| | {[(methylamino)carbonothioyl]amino}phenyl)-4- | | | |
| | morpholin-4-yl-1H-pyrazolo[3,4-d]pyrimidin-1- | | | |
| | yl]piperidine-1-carboxylate | | | |
| 743 | 1-(4-{1-[1-(4-hydroxybutyl)piperidin-4-yl]-4-morpholin- | 509.3 | 1.82 | 40 |
| | 4-yl-1H-pyrazolo[3,4-d]pyrimidin-6-yl}phenyl)-3- | | | |
| | methylurea | | | |
| 744 | methyl (4-{1-[1-(4-hydroxybutyl)piperidin-4-yl]-4- | 510.3 | 1.92 | 40 |
| | morpholin-4-yl-1H-pyrazolo[3,4-d]pyrimidin-6- | | | |
| | yl}phenyl)carbamate | | | |
| 745 | 1-ethyl-3-(4-{1-[1-(4-hydroxybutyl)piperidin-4-yl]-4- | 523.3 | 1.87 | 40 |
| | morpholin-4-yl-1H-pyrazolo[3,4-d]pyrimidin-6- | | | |
| | yl}phenyl)urea | | | |
| 746 | 1-(4-{1-[1-(4-hydroxybutyl)piperidin-4-yl]-4-morpholin- | 539.3 | 1.79 | 40 |
| | 4-yl-1H-pyrazolo[3,4-d]pyrimidin-6-yl}phenyl)-3-(2- | | | |
| | hydroxyethyl)urea | | | |
| 747 | 1-(2-fluoroethyl)-3-(4-{1-[1-(4-hydroxybutyl)piperidin-4- | 541.3 | 1.86 | 40 |
| | yl]-4-morpholin-4-yl-1H-pyrazolo[3,4-d]pyrimidin-6- | | | |
| | yl}phenyl)urea | | | |
| 748 | 1-(4-{1-[1-(4-hydroxybutyl)piperidin-4-yl]-4-morpholin- | 571.5 | 2.07 | 40 |
| | 4-yl-1H-pyrazolo[3,4-d]pyrimidin-6-yl}phenyl)-3- | | | |
| | phenylurea | | | |
| 749 | 4-{4-[6-(4-aminophenyl)-4-morpholin-4-yl-1H- | 452.3 | 1.74 | 40 |
| | pyrazolo[3,4-d]pyrimidin-1-yl]piperidin-1-yl}butan-1-ol | | | |
| 750 | ethyl 4-(6-{4-[(methylcarbamoyl)amino]phenyl}-4- | 509.3 | 2.27 | 23, 3 |
| | morpholin-4-yl-1H-pyrazolo[3,4-d]pyrimidin-1- | | | |
| | yl)piperidine-1-carboxylate | | | |
| 751 | propyl 4-(6-{4-[(methylcarbamoyl)amino]phenyl}-4- | 523.3 | 2.35 | 23, 3 |
| | morpholin-4-yl-1H-pyrazolo[3,4-d]pyrimidin-1- | | | |
| | yl)piperidine-1-carboxylate | | | |
| 752 | isopropyl 4-(6-{4-[(methylcarbamoyl)amino]phenyl}-4- | 523.3 | 2.34 | 23, 3 |
| | morpholin-4-yl-1H-pyrazolo[3,4-d]pyrimidin-1- | | | |
| | yl)piperidine-1-carboxylate | | | |
| 753 | vinyl 4-(6-{4-[(methylcarbamoyl)amino]phenyl}-4- | 507.2 | 2.28 | 23, 3 |
| | morpholin-4-yl-1H-pyrazolo[3,4-d]pyrimidin-1- | | | |
| | yl)piperidine-1-carboxylate | | | |
| 754 | isobutyl 4-(6-{4-[(methylcarbamoyl)amino]phenyl}-4- | 537.3 | 2.42 | 23, 3 |
| | morpholin-4-yl-1H-pyrazolo[3,4-d]pyrimidin-1- | | | |
| | yl)piperidine-1-carboxylate | | | |
| 755 | phenyl 4-(6-{4-[(methylcarbamoyl)amino]phenyl}-4- | 557.3 | 2.38 | 23, 3 |
| | morpholin-4-yl-1H-pyrazolo[3,4-d]pyrimidin-1- | | | |
| | yl)piperidine-1-carboxylate | | | |
| 756 | 1-[4-(1-{1-[(2E)-but-2-enoyl]piperidin-4-yl}-4- | 505.3 | 2.19 | 23, 7c |
| | morpholin-4-yl-1H-pyrazolo[3,4-d]pyrimidin-6- | | | |
| | yl)phenyl]-3-methylurea | | | |
| 757 | methyl 3-[4-(6-{4-[(methylcarbamoyl)amino]phenyl}-4- | 537.2 | 2.1 | 23, 7c |
| | morpholin-4-yl-1H-pyrazolo[3,4-d]pyrimidin-1- | | | |
| | yl)piperidin-1-yl]-3-oxopropanoate | | | |
| 758 | 1-{4-[1-(1-acryloylpiperidin-4-yl)-4-morpholin-4-yl-1H- | 491.2 | 2.14 | 23, 7c |
| | pyrazolo[3,4-d]pyrimidin-6-yl]phenyl}-3-methylurea | | | |
| 759 | 1-methyl-3-(4-{1-[1-(methylsulfonyl)piperidin-4-yl]-4- | 515.2 | 2.12 | 23, 7c |
| | morpholin-4-yl-1H-pyrazolo[3,4-d]pyrimidin-6- | | | |
| | yl}phenyl)urea | | | |
| 760 | N-methyl-4-(6-{4-[(methylcarbamoyl)amino]phenyl}-4- | 510.2 | 2.12 | 23, 10 |
| | morpholin-4-yl-1H-pyrazolo[3,4-d]pyrimidin-1- | | | |
| | yl)piperidine-1-carbothioamide | | | |
| 761 | S-methyl 4-(6-{4-[(methylcarbamoyl)amino]phenyl}-4- | 511.2 | 2.28 | 23, 10 |
| | morpholin-4-yl-1H-pyrazolo[3,4-d]pyrimidin-1- | | | |
| | yl)piperidine-1-carbothioate | | | |
| 762 | S-ethyl 4-(6-{4-[(methylcarbamoyl)amino]phenyl}-4- | 525.2 | 2.35 | 23, 10 |
| | morpholin-4-yl-1H-pyrazolo[3,4-d]pyrimidin-1- | | | |
| | yl)piperidine-1-carbothioate | | | |
| 763 | 1-methyl-3-(4-{4-morpholin-4-yl-1-[1- | | | 23, 7c |
| | (trifluoroacetyl)piperidin-4-yl]-1H-pyrazolo[3,4- | | | |
| | d]pyrimidin-6-yl}phenyl)urea | | | |
| 764 | tert-butyl 4-(6-{4-[(ethylcarbamoyl)amino]phenyl}-4- | 551.3 | 2.5 | 23 |
| | morpholin-4-yl-1H-pyrazolo[3,4-d]pyrimidin-1- | | | |
| | yl)piperidine-1-carboxylate | | | |
| 765 | tert-butyl 4-[6-(4-{[(2- | 569.3 | 2.46 | 23 |
| | fluoroethyl)carbamoyl]amino}phenyl)-4-morpholin-4-yl- | | | |
| | 1H-pyrazolo[3,4-d]pyrimidin-1-yl]piperidine-1- | | | |
| | carboxylate | | | |
| 766 | tert-butyl 4-[6-(4-{[(2- | 567.3 | 2.34 | 23 |
| | hydroxyethyl)carbamoyl]amino}phenyl)-4-morpholin-4- | | | |
| | yl-1H-pyrazolo[3,4-d]pyrimidin-1-yl]piperidine-1- | | | |
| | carboxylate | | | |
| 767 | tert-butyl 4-(6-{4- | 563.3 | 2.52 | 23 |
| | [(cyclopropylcarbamoyl)amino]phenyl}-4-morpholin-4- | | | |
| | yl-1H-pyrazolo[3,4-d]pyrimidin-1-yl)piperidine-1- | | | |
| | carboxylate | | | |
| 768 | tert-butyl 4-(6-{4-[(anilinocarbonyl)amino]phenyl}-4- | 599.3 | 2.67 | 23 |
| | morpholin-4-yl-1H-pyrazolo[3,4-d]pyrimidin-1- | | | |
| | yl)piperidine-1-carboxylate | | | |
| 769 | tert-butyl 4-(4-morpholin-4-yl-6-{4-[(pyridin-2- | 600.3 | 2.75 | 23 |
| | ylcarbamoyl)amino]phenyl}-1H-pyrazolo[3,4- | | | |
| | d]pyrimidin-1-yl)piperidine-1-carboxylate | | | |
| 770 | tert-butyl 4-(4-morpholin-4-yl-6-{4-[(pyridin-3- | 600.3 | 2.31 | 23 |
| | ylcarbamoyl)amino]phenyl}-1H-pyrazolo[3,4- | | | |
| | d]pyrimidin-1-yl)piperidine-1-carboxylate | | | |
| 771 | tert-butyl 4-(4-morpholin-4-yl-6-{4-[(pyridin-4- | 600.3 | 2.23 | 23 |
| | ylcarbamoyl)amino]phenyl}-1H-pyrazolo[3,4- | | | |
| | d]pyrimidin-1-yl)piperidine-1-carboxylate | | | |
| 772 | tert-butyl 4-(6-{4-[(methoxycarbonyl)amino]phenyl}-4- | 538.3 | 2.62 | 23 |
| | morpholin-4-yl-1H-pyrazolo[3,4-d]pyrimidin-1- | | | |
| | yl)piperidine-1-carboxylate | | | |
| 773 | 1-ethyl-3-[4-(4-morpholin-4-yl-1-piperidin-4-yl-1H | 451.2 | 1.78 | 41 |
| | pyrazolo[3,4-d]pyrimidin-6-yl)phenyl]urea | | | |
| 774 | 1-(2-fluoroethyl)-3-[4-(4-morpholin-4-yl-1-piperidin-4-yl- | 469.2 | 1.77 | 41 |
| | 1H-pyrazolo[3,4-d]pyrimidin-6-yl)phenyl]urea | | | |
| 775 | 1-(2-hydroxyethyl)-3-[4-(4-morpholin-4-yl-1-piperidin-4- | 467.2 | 1.68 | 41 |
| | yl-1H-pyrazolo[3,4-d]pyrimidin-6-yl)phenyl]urea | | | |
| 776 | 1-cyclopropyl-3-[4-(4-morpholin-4-yl-1-piperidin-4-yl- | 463.2 | 1.79 | 41 |
| | 1H-pyrazolo[3,4-d]pyrimidin-6-yl)phenyl]urea | | | |
| 777 | 1-[4-(4-morpholin-4-yl-1-piperidin-4-yl-1H-pyrazolo[3,4- | 499.2 | 1.98 | 41 |
| | d]pyrimidin-6-yl)phenyl]-3-phenylurea | | | |
| 778 | 1-[4-(4-morpholin-4-yl-1-piperidin-4-yl-1H-pyrazolo[3,4- | 500.2 | 1.93 | 41 |
| | d]pyrimidin-6-yl)phenyl]-3-pyridin-2-ylurea | | | |
| 779 | 1-[4-(4-morpholin-4-yl-1-piperidin-4-yl-1H-pyrazolo[3,4- | 500.2 | 1.67 | 41 |
| | d]pyrimidin-6-yl)phenyl]-3-pyridin-3-ylurea | | | |
| 780 | 1-[4-(4-morpholin-4-yl-1-piperidin-4-yl-1H-pyrazolo[3,4- | 500.2 | 1.65 | 41 |
| | d]pyrimidin-6-yl)phenyl]-3-pyridin-4-ylurea | | | |
| 781 | methyl (4-{4-morpholin-4-yl-1-[1-(2,2,2- | 520.2 | 2.54 | 48 |
| | trifluoroethyl)piperidin-4-yl]-1H-pyrazolo[3,4- | | | |
| | d]pyrimidin-6-yl}phenyl)carbamate | | | |
| 782 | 1-ethyl-3-(4-{4-morpholin-4-yl-1-[1-(2,2,2- | 533.3 | 2.41 | 48 |
| | trifluoroethyl)piperidin-4-yl]-1H-pyrazolo[3,4- | | | |
| | d]pyrimidin-6-yl}phenyl)urea | | | |
| 783 | 1-(2-fluoroethyl)-3-(4-{4-morpholin-4-yl-1-[1-(2,2,2- | 551.2 | 2.31 | 48 |
| | trifluoroethyl)piperidin-4-yl]-1H-pyrazolo[3,4- | | | |
| | d]pyrimidin-6-yl}phenyl)urea | | | |
| 784 | 1-(2-hydroxyethyl)-3-(4-{4-morpholin-4-yl-1-[1-(2,2,2- | 549.2 | 2.14 | 48 |
| | trifluoroethyl)piperidin-4-yl]-1H-pyrazolo[3,4- | | | |
| | d]pyrimidin-6-yl}phenyl)urea | | | |
| 785 | 1-(4-{4-morpholin-4-yl-1-[1-(2,2,2- | 582.2 | 2.62 | 48 |
| | trifluoroethyl)piperidin-4-yl]-1H-pyrazolo[3,4- | | | |
| | d]pyrimidin-6-yl}phenyl)-3-pyridin-2-ylurea | | | |
| 786 | 1-(4-{4-morpholin-4-yl-1-[1-(2,2,2- | 582.2 | 2.11 | 48 |
| | trifluoroethyl)piperidin-4-yl]-1H-pyrazolo[3,4- | | | |
| | d]pyrimidin-6-yl}phenyl)-3-pyridin-4-ylurea | | | |
| 787 | 1-(4-{4-morpholin-4-yl-1-[1-(2,2,2- | 582.2 | 2.15 | 48 |
| | trifluoroethyl)piperidin-4-yl]-1H-pyrazolo[3,4- | | | |
| | d]pyrimidin-6-yl}phenyl)-3-pyridin-3-ylurea | | | |
| 788 | 1-cyclopropyl-3-(4-{4-morpholin-4-yl-1-[1-(2,2,2- | 545.3 | 2.36 | 48 |
| | trifluoroethyl)piperidin-4-yl]-1H-pyrazolo[3,4- | | | |
| | d]pyrimidin-6-yl}phenyl)urea | | | |
| 789 | 1-(4-{4-morpholin-4-yl-1-[1-(2,2,2- | 581.3 | 2.57 | 48 |
| | trifluoroethyl)piperidin-4-yl]-1H-pyrazolo[3,4- | | | |
| | d]pyrimidin-6-yl}phenyl)-3-phenylurea | | | |
| 790 | 1-(2-fluoroethyl)-3-{4-[4-morpholin-4-yl-1-(2,2,2- | 468.2 | 2.24 | 56 |
| | trifluoroethyl)-1H-pyrazolo[3,4-d]pyrimidin-6- | | | |
| | yl]phenyl}urea | | | |
| 791 | 1-(2-hydroxyethyl)-3-{4-[4-morpholin-4-yl-1-(2,2,2- | 466.2 | 2.13 | 56 |
| | trifluoroethyl)-1H-pyrazolo[3,4-d]pyrimidin-6- | | | |
| | yl]phenyl}urea | | | |
| 792 | 1-{4-[4-morpholin-4-yl-1-(2,2,2-trifluoroethyl)-1H- | 499.2 | 2.13 | 56 |
| | pyrazolo[3,4-d]pyrimidin-6-yl]phenyl}-3-pyridin-3-ylurea | | | |
| 793 | 1-(4-{1-[1-(cyanomethyl)piperidin-4-yl]-4-morpholin-4- | 476.2 | 2.12 | 23, 16 |
| | yl-1H-pyrazolo[3,4-d]pyrimidin-6-yl}phenyl)-3- | | | |
| | methylurea | | | |
| 794 | methyl [4-(6-{4-[(methylcarbamoyl)amino]phenyl}-4- | 509.3 | 2.17 | 23, 16 |
| | morpholin-4-yl-1H-pyrazolo[3,4-d]pyrimidin-1- | | | |
| | yl)piperidin-1-yl]acetate | | | |
| 795 | ethyl [4-(6-{4-[(methylcarbamoyl)amino]phenyl}-4- | 523.3 | 1.85 | 23, 16 |
| | morpholin-4-yl-1H-pyrazolo[3,4-d]pyrimidin-1- | | | |
| | yl)piperidin-1-yl]acetate | | | |
| 796 | 1-(4-{1-[1-(methoxymethyl)piperidin-4-yl]-4-morpholin- | 479.4 | 1.8 | 23, 16 |
| | 4-yl-1H-pyrazolo[3,4-d]pyrimidin-6-yl}phenyl)-3- | | | |
| | methylurea | | | |
| 797 | 1-(4-{1-[1-(1,3-dioxolan-2-ylmethyl)piperidin-4-yl]-4- | 523.3 | 1.82 | 23, 16 |
| | morpholin-4-yl-1H-pyrazolo[3,4-d]pyrimidin-6- | | | |
| | yl}phenyl)-3-methylurea | | | |
| 798 | [4-(6-{4-[(methylcarbamoyl)amino]phenyl}-4-morpholin- | 495.2 | 1.85 | 23, 16 |
| | 4-yl-1H-pyrazolo[3,4-d]pyrimidin-1-yl)piperidin-1- | | | |
| | yl]acetic acid | | | |
| 799 | 1-{4-[1-(1-allylpiperidin-4-yl)-4-morpholin-4-yl-1H- | 477.3 | 1.81 | 23, 16 |
| | pyrazolo[3,4-d]pyrimidin-6-yl]phenyl}-3-methylurea | | | |
| 800 | 2-methoxyethyl 4-(6-{4- | 539.3 | 2.2 | 23, 7c |
| | [(methylcarbamoyl)amino]phenyl}-4-morpholin-4-yl-1H- | | | |
| | pyrazolo[3,4-d]pyrimidin-1-yl)piperidine-1-carboxylate | | | |
| 801 | but-2-yn-1-yl 4-(6-{4-[(methylcarbamoyl)amino]phenyl}- | 533.3 | 2.32 | 23, 7c |
| | 4-morpholin-4-yl-1H-pyrazolo[3,4-d]pyrimidin-1- | | | |
| | yl)piperidine-1-carboxylate | | | |
| 802 | 2-(methylamino)ethyl 4-(6-{4- | 538.3 | 1.82 | 23, 7c |
| | [(methylcarbamoyl)amino]phenyl}-4-morpholin-4-yl-1H- | | | |
| | pyrazolo[3,4-d]pyrimidin-1-yl)piperidine-1-carboxylate | | | |
| 803 | 2-(dimethylamino)ethyl 4-(6-{4- | 552.3 | 1.85 | 23, 7c |
| | [(methylcarbamoyl)amino]phenyl}-4-morpholin-4-yl-1H- | | | |
| | pyrazolo[3,4-d]pyrimidin-1-yl)piperidine-1-carboxylate | | | |
| 804 | 2-bromoethyl 4-(6-{4-[(methylcarbamoyl)amino]phenyl}- | 587.2 | 2.32 | 23, 7c |
| | 4-morpholin-4-yl-1H-pyrazolo[3,4-d]pyrimidin-1- | | | |
| | yl)piperidine-1-carboxylate | | | |
| 805 | ethyl 4-(6-{4-[(methoxycarbonyl)amino]phenyl}-4- | 510.2 | 2.45 | 23, 3 |
| | morpholin-4-yl-1H-pyrazolo[3,4-d]pyrimidin-1- | | | |
| | yl)piperidine-1-carboxylate | | | |
| 806 | isopropyl 4-(6-{4-[(methoxycarbonyl)amino]phenyl}-4- | 524.3 | 2.52 | 23, 3 |
| | morpholin-4-yl-1H-pyrazolo[3,4-d]pyrimidin-1- | | | |
| | yl)piperidine-1-carboxylate | | | |
| 807 | S-ethyl 4-(6-{4-[(methoxycarbonyl)amino]phenyl}-4- | 526.2 | 2.52 | 23, 3 |
| | morpholin-4-yl-1H-pyrazolo[3,4-d]pyrimidin-1- | | | |
| | yl)piperidine-1-carbothioate | | | |
| 808 | methyl (4-{1-[1-(dimethylcarbamoyl)piperidin-4-yl]-4- | 509.3 | 2.38 | 23, 10 |
| | morpholin-4-yl-1H-pyrazolo[3,4-d]pyrimidin-6- | | | |
| | yl}phenyl)carbamate | | | |
| 809 | methyl {4-[1-(1-acetylpiperidin-4-yl)-4-morpholin-4-yl- | 480.2 | 2.27 | 23, 7c |
| | 1H-pyrazolo[3,4-d]pyrimidin-6-yl]phenyl}carbamate | | | |
| 810 | methyl {4-[1-(1-isobutyrylpiperidin-4-yl)-4-morpholin-4- | 508.3 | 2.38 | 23, 7c |
| | yl-1H-pyrazolo[3,4-d]pyrimidin-6-yl]phenyl}carbamate | | | |
| 811 | methyl (4-{4-morpholin-4-yl-1-[1- | 534.2 | 2.43 | 23, 7c |
| | (trifluoroacetyl)piperidin-4-yl]-1H-pyrazolo[3,4- | | | |
| | d]pyrimidin-6-yl}phenyl)carbamate | | | |
| 812 | methyl [4-(1-{1-[(ethylamino)carbonothioyl]piperidin-4- | 525.2 | 2.31 | 23, 10 |
| | yl}-4-morpholin-4-yl-1H-pyrazolo[3,4-d]pyrimidin-6- | | | |
| | yl)phenyl]carbamate | | | |
| 813 | methyl (4-{1-[1-(methylcarbamoyl)piperidin-4-yl]-4- | 495.2 | 2.21 | 23, 10 |
| | morpholin-4-yl-1H-pyrazolo[3,4-d]pyrimidin-6- | | | |
| | yl}phenyl)carbamate | | | |
| 814 | ethyl 4-(6-{4-[(anilinocarbonyl)amino]phenyl}-4- | 571.3 | 2.54 | 1, 2, 22 |
| | morpholin-4-yl-1H-pyrazolo[3,4-d]pyrimidin-1- | | | |
| | yl)piperidine-1-carboxylate | | | |
| 815 | ethyl 4-(4-morpholin-4-yl-6-{4-[(pyridin-2- | 572.3 | 2.58 | 1, 2, 22 |
| | ylcarbamoyl)amino]phenyl}-1H-pyrazolo[3,4- | | | |
| | d]pyrimidin-1-yl)piperidine-1-carboxylate | | | |
| 816 | ethyl 4-(4-morpholin-4-yl-6-{4-[(pyridin-3- | 572.3 | 2.16 | 1, 2, 22 |
| | ylcarbamoyl)amino]phenyl}-1H-pyrazolo[3,4- | | | |
| | d]pyrimidin-1-yl)piperidine-1-carboxylate | | | |
| 817 | ethyl 4-(4-morpholin-4-yl-6-{4-[(pyridin-4- | 572.3 | 2.1 | 1, 2, 22 |
| | ylcarbamoyl)amino]phenyl}-1H-pyrazolo[3,4- | | | |
| | d]pyrimidin-1-yl)piperidine-1-carboxylate | | | |
| 818 | ethyl 4-[6-(4-{[(2- | 539.3 | 2.18 | 1, 2, 22 |
| | hydroxyethyl)carbamoyl]amino}phenyl)-4-morpholin-4- | | | |
| | yl-1H-pyrazolo[3,4-d]pyrimidin-1-yl]piperidine-1- | | | |
| | carboxylate | | | |
| 819 | ethyl 4-[6-(4-{[(2-fluoroethyl)carbamoyl]amino}phenyl)- | 541.3 | 2.32 | 1, 2, 22 |
| | 4-morpholin-4-yl-1H-pyrazolo[3,4-d]pyrimidin-1- | | | |
| | yl]piperidine-1-carboxylate | | | |
| 820 | ethyl 4-(6-{4-[(ethylcarbamoyl)amino]phenyl}-4- | 523.3 | 2.34 | 1, 2, 22 |
| | morpholin-4-yl-1H-pyrazolo[3,4-d]pyrimidin-1- | | | |
| | yl)piperidine-1-carboxylate | | | |
| 821 | ethyl 4-(6-{4-[(cyclopropylcarbamoyl)amino]phenyl}-4- | 535.3 | 2.36 | 1, 2, 22 |
| | morpholin-4-yl-1H-pyrazolo[3,4-d]pyrimidin-1- | | | |
| | yl)piperidine-1-carboxylate | | | |
| 822 | 1-ethyl-3-{4-[1-(1-isobutyrylpiperidin-4-yl)-4-morpholin- | 521.3 | 2.25 | 7c, 23 |
| | 4-yl-1H-pyrazolo[3,4-d]pyrimidin-6-yl]phenyl}urea | | | |
| 823 | 1-(2-hydroxyethyl)-3-{4-[1-(1-isobutyrylpiperidin-4-yl)- | 537.3 | 2.04 | 7c, 23 |
| | 4-morpholin-4-yl-1H-pyrazolo[3,4-d]pyrimidin-6- | | | |
| | yl]phenyl}urea | | | |
| 824 | 1-cyclopropyl-3-{4-[1-(1-isobutyrylpiperidin-4-yl)-4- | 533.3 | 2.27 | 7c, 23 |
| | morpholin-4-yl-1H-pyrazolo[3,4-d]pyrimidin-6- | | | |
| | yl]phenyl}urea | | | |
| 825 | 1-(2-fluoroethyl)-3-{4-[1-(1-isobutyrylpiperidin-4-yl)-4- | 539.3 | 2.21 | 7c, 23 |
| | morpholin-4-yl-1H-pyrazolo[3,4-d]pyrimidin-6- | | | |
| | yl]phenyl}urea | | | |
| 826 | 1-{4-[1-(1-isobutyrylpiperidin-4-yl)-4-morpholin-4-yl- | 569.3 | 2.49 | 7c, 23 |
| | 1H-pyrazolo[3,4-d]pyrimidin-6-yl]phenyl}-3-phenylurea | | | |
| 827 | 1-{4-[1-(1-isobutyrylpiperidin-4-yl)-4-morpholin-4-yl- | 570.3 | 2.52 | 7c, 23 |
| | 1H-pyrazolo[3,4-d]pyrimidin-6-yl]phenyl}-3-pyridin-2- | | | |
| | ylurea | | | |
| 828 | 1-{4-[1-(1-isobutyrylpiperidin-4-yl)-4-morpholin-4-yl- | 570.3 | 1.99 | 7c, 23 |
| | 1H-pyrazolo[3,4-d]pyrimidin-6-yl]phenyl}-3-pyridin-3- | | | |
| | ylurea | | | |
| 829 | 1-{4-[1-(1-isobutyrylpiperidin-4-yl)-4-morpholin-4-yl- | 570.3 | 1.91 | 7c, 23 |
| | 1H-pyrazolo[3,4-d]pyrimidin-6-yl]phenyl}-3-pyridin-4- | | | |
| | ylurea | | | |
| 830 | isopropyl 4-[6-(4-aminophenyl)-4-morpholin-4-yl-1H- | 466.2 | 2.35 | 23 |
| | pyrazolo[3,4-d]pyrimidin-1-yl]piperidine-1-carboxylate | | | |
| 831 | isopropyl 4-[6-(4- | 539.2 | 2.4 | 15 |
| | {[(methylamino)carbonothioyl]amino}phenyl)-4- | | | |
| | morpholin-4-yl-1H-pyrazolo[3,4-d]pyrimidin-1- | | | |
| | yl]piperidine-1-carboxylate | | | |
| 832 | isopropyl 4-[6-(4-{[(1E)- | 553.3 | 2.02 | 42 |
| | (methylamino)(methylthio)methylene]amino}phenyl)-4- | | | |
| | morpholin-4-yl-1H-pyrazolo[3,4-d]pyrimidin-1- | | | |
| | yl]piperidine-1-carboxylate | | | |
| 833 | isopropyl 4-[6-(4-{[(2- | 555.3 | 2.38 | 23 |
| | fluoroethyl)carbamoyl]amino}phenyl)-4-morpholin-4-yl- | | | |
| | 1H-pyrazolo[3,4-d]pyrimidin-1-yl]piperidine-1- | | | |
| | carboxylate | | | |
| 834 | isopropyl 4-[6-(4-{[(2- | 553.3 | 2.21 | 23 |
| | hydroxyethyl)carbamoyl]amino}phenyl)-4-morpholin-4- | | | |
| | yl-1H-pyrazolo[3,4-d]pyrimidin-1-yl]piperidine-1- | | | |
| | carboxylate | | | |
| 835 | isopropyl 4-(6-{4- | 549.3 | 2.43 | 23 |
| | [(cyclopropylcarbamoyl)amino]phenyl}-4-morpholin-4- | | | |
| | yl-1H-pyrazolo[3,4-d]pyrimidin-1-yl)piperidine-1- | | | |
| | carboxylate | | | |
| 836 | isopropyl 4-(6-{4-[(anilinocarbonyl)amino]phenyl}-4- | 585.3 | 2.62 | 23 |
| | morpholin-4-yl-1H-pyrazolo[3,4-d]pyrimidin-1- | | | |
| | yl)piperidine-1-carboxylate | | | |
| 837 | isopropyl 4-(4-morpholin-4-yl-6-{4-[(pyridin-2- | 586.3 | 2.69 | 23 |
| | ylcarbamoyl)amino]phenyl}-1H-pyrazolo[3,4- | | | |
| | d]pyrimidin-1-yl)piperidine-1-carboxylate | | | |
| 838 | isopropyl 4-(4-morpholin-4-yl-6-{4-[(pyridin-3- | 586.3 | 2.16 | 23 |
| | ylcarbamoyl)amino]phenyl}-1H-pyrazolo[3,4- | | | |
| | d]pyrimidin-1-yl)piperidine-1-carboxylate | | | |
| 839 | isopropyl 4-(4-morpholin-4-yl-6-{4-[(pyridin-4- | 586.3 | 2.06 | 23 |
| | ylcarbamoyl)amino]phenyl}-1H-pyrazolo[3,4- | | | |
| | d]pyrimidin-1-yl)piperidine-1-carboxylate | | | |
| 840 | methyl 4-[6-{4-[(methoxycarbonyl)amino]phenyl}-4-(2- | 510.2 | 2.48 | 49 |
| | methylmorpholin-4-yl)-1H-pyrazolo[3,4-d]pyrimidin-1- | | | |
| | yl]piperidine-1-carboxylate | | | |
| 841 | methyl 4-[6-{4-[(methylcarbamoyl)amino]phenyl}-4-(2- | 509.3 | 2.25 | 49 |
| | methylmorpholin-4-yl)-1H-pyrazolo[3,4-d]pyrimidin-1- | | | |
| | yl]piperidine-1-carboxylate | | | |
| 842 | methyl 4-[6-{4-[(ethylcarbamoyl)amino]phenyl}-4-(2- | 523.3 | 2.33 | 49 |
| | methylmorpholin-4-yl)-1H-pyrazolo[3,4-d]pyrimidin-1- | | | |
| | yl]piperidine-1-carboxylate | | | |
| 843 | methyl 4-[6-{4-[(cyclopropylcarbamoyl)amino]phenyl}- | 535.3 | 2.35 | 49 |
| | 4-(2-methylmorpholin-4-yl)-1H-pyrazolo[3,4- | | | |
| | d]pyrimidin-1-yl]piperidine-1-carboxylate | | | |
| 844 | methyl 4-[6-(4-{[(2- | 541.3 | 2.29 | 49 |
| | fluoroethyl)carbamoyl]amino}phenyl)-4-(2- | | | |
| | methylmorpholin-4-yl)-1H-pyrazolo[3,4-d]pyrimidin-1- | | | |
| | yl]piperidine-1-carboxylate | | | |
| 845 | methyl 4-[6-(4-{[(2- | 539.3 | 2.12 | 49 |
| | hydroxyethyl)carbamoyl]amino}phenyl)-4-(2- | | | |
| | methylmorpholin-4-yl)-1H-pyrazolo[3,4-d]pyrimidin-1- | | | |
| | yl]piperidine-1-carboxylate | | | |
| 846 | methyl 4-[4-(2-methylmorpholin-4-yl)-6-{4-[(pyridin-3- | 572.3 | 2.07 | 49 |
| | ylcarbamoyl)amino]phenyl}-1H-pyrazolo[3,4- | | | |
| | d]pyrimidin-1-yl]piperidine-1-carboxylate | | | |
| 847 | methyl 4-[4-(2-methylmorpholin-4-yl)-6-{4-[(pyridin-2- | 572.3 | 2.61 | 49 |
| | ylcarbamoyl)amino]phenyl}-1H-pyrazolo[3,4- | | | |
| | d]pyrimidin-1-yl]piperidine-1-carboxylate | | | |
| 848 | methyl 4-[6-{4-[(anilinocarbonyl)amino]phenyl}-4-(2- | 571.3 | 2.56 | 49 |
| | methylmorpholin-4-yl)-1H-pyrazolo[3,4-d]pyrimidin-1- | | | |
| | yl]piperidine-1-carboxylate | | | |
| 849 | propyl 4-(4-morpholin-4-yl-6-{4- | 585.3 | 2.63 | 1, 2, 22 |
| | [(phenylcarbamoyl)amino]phenyl}-1H-pyrazolo[3,4- | | | |
| | d]pyrimidin-1-yl)piperidine-1-carboxylate | | | |
| 850 | propyl 4-(4-morpholin-4-yl-6-{4-[(pyridin-3- | 586.3 | 2.17 | 1, 2, 22 |
| | ylcarbamoyl)amino]phenyl}-1H-pyrazolo[3,4- | | | |
| | d]pyrimidin-1-yl)piperidine-1-carboxylate | | | |
| 851 | propyl 4-(4-morpholin-4-yl-6-{4-[(pyridin-4- | 586.3 | 2.05 | 1, 2, 22 |
| | ylcarbamoyl)amino]phenyl}-1H-pyrazolo[3,4- | | | |
| | d]pyrimidin-1-yl)piperidine-1-carboxylate | | | |
| 852 | propyl 4-(6-{4-[(ethylcarbamoyl)amino]phenyl}-4- | 537.3 | 2.42 | 1, 2, 22 |
| | morpholin-4-yl-1H-pyrazolo[3,4-d]pyrimidin-1- | | | |
| | yl)piperidine-1-carboxylate | | | |
| 853 | propyl 4-(4-morpholin-4-yl-6-{4- | 551.3 | 2.5 | 1, 2, 22 |
| | [(propylcarbamoyl)amino]phenyl}-1H-pyrazolo[3,4- | | | |
| | d]pyrimidin-1-yl)piperidine-1-carboxylate | | | |
| 854 | propyl 4-[6-(4-{[(2- | 555.3 | 2.39 | 1, 2, 22 |
| | fluoroethyl)carbamoyl]amino}phenyl)-4-morpholin-4-yl- | | | |
| | 1H-pyrazolo[3,4-d]pyrimidin-1-yl]piperidine-1- | | | |
| | carboxylate | | | |
| 855 | propyl 4-[6-(4-{[(2- | 553.3 | 2.22 | 1, 2, 22 |
| | hydroxyethyl)carbamoyl]amino}phenyl)-4-morpholin-4- | | | |
| | yl-1H-pyrazolo[3,4-d]pyrimidin-1-yl]piperidine-1- | | | |
| | carboxylate | | | |
| 856 | propyl 4-(6-{4-[(cyclopropylcarbamoyl)amino]phenyl}-4- | 549.3 | 2.44 | 1, 2, 22 |
| | morpholin-4-yl-1H-pyrazolo[3,4-d]pyrimidin-1- | | | |
| | yl)piperidine-1-carboxylate | | | |
| 857 | propyl 4-(6-{4-[(methoxycarbonyl)amino]phenyl}-4- | 524.3 | 2.56 | 1, 2, 22 |
| | morpholin-4-yl-1H-pyrazolo[3,4-d]pyrimidin-1- | | | |
| | yl)piperidine-1-carboxylate | | | |
| 858 | propyl 4-[6-(4- | 563.3 | 2.51 | 1, 2, 22 |
| | {[(cyclopropylmethyl)carbamoyl]amino}phenyl)-4- | | | |
| | morpholin-4-yl-1H-pyrazolo[3,4-d]pyrimidin-1- | | | |
| | yl]piperidine-1-carboxylate | | | |
| 859 | methyl 4-[6-(4-{[(4- | 575.2 | 2.42 | 1, 2, 23 |
| | fluorophenyl)carbamoyl]amino}phenyl)-4-morpholin-4- | | | |
| | yl-1H-pyrazolo[3,4-d]pyrimidin-1-yl]piperidine-1- | | | |
| | carboxylate | | | |
| 860 | methyl 4-[6-(4-{[(2- | 575.2 | 2.48 | 1, 2, 23 |
| | fluorophenyl)carbamoyl]amino}phenyl)-4-morpholin-4- | | | |
| | yl-1H-pyrazolo[3,4-d]pyrimidin-1-yl]piperidine-1- | | | |
| | carboxylate | | | |
| 861 | methyl 4-[6-(4-{[(2,4- | 593.2 | 2.49 | 1, 2, 23 |
| | difluorophenyl)carbamoyl]amino}phenyl)-4-morpholin-4- | | | |
| | yl-1H-pyrazolo[3,4-d]pyrimidin-1-yl]piperidine-1- | | | |
| | carboxylate | | | |
| 862 | methyl 4-[6-(4-{[(6-fluoropyridin-3- | 576.2 | 2.31 | 1, 2, 23 |
| | yl)carbamoyl]amino}phenyl)-4-morpholin-4-yl-1H- | | | |
| | pyrazolo[3,4-d]pyrimidin-1-yl]piperidine-1-carboxylate | | | |
| 863 | methyl 4-[6-(4-{[(2-fluoropyridin-3- | 576.2 | 2.4 | 1, 2, 23 |
| | yl)carbamoyl]amino}phenyl)-4-morpholin-4-yl-1H- | | | |
| | pyrazolo[3,4-d]pyrimidin-1-yl]piperidine-1-carboxylate | | | |
| 864 | methyl 4-[6-(4-{[(3-fluoropyridin-4- | 576.2 | 2.14 | 1, 2, 23 |
| | yl)carbamoyl]amino}phenyl)-4-morpholin-4-yl-1H- | | | |
| | pyrazolo[3,4-d]pyrimidin-1-yl]piperidine-1-carboxylate | | | |
| 865 | methyl 4-[6-(4-{[(2- | 528.2 | 2.32 | 1, 2, 23 |
| | fluoroethoxy)carbonyl]amino}phenyl)-4-morpholin-4-yl- | | | |
| | 1H-pyrazolo[3,4-d]pyrimidin-1-yl]piperidine-1- | | | |
| | carboxylate | | | |
| 866 | methyl 4-[6-(4-{[(2- | 576.2 | 2.51 | 1, 2, 23 |
| | fluorophenoxy)carbonyl]amino}phenyl)-4-morpholin-4- | | | |
| | yl-1H-pyrazolo[3,4-d]pyrimidin-1-yl]piperidine-1- | | | |
| | carboxylate | | | |
| 867 | 1-methyl-3-{4-[4-morpholin-4-yl-1-(tetrahydro-2H- | 454.2 | 2.19 | 3, 23 |
| | thiopyran-4-yl)-1H-pyrazolo[3,4-d]pyrimidin-6- | | | |
| | yl]phenyl}urea | | | |
| 868 | 1-methyl-3-{4-[4-morpholin-4-yl-1-(1-oxidotetrahydro- | 470.2 | 1.9 | 3, 23 |
| | 2H-thiopyran-4-yl)-1H-pyrazolo[3,4-d]pyrimidin-6- | | | |
| | yl]phenyl}urea | | | |
| 869 | 1-{4-[1-(1,1-dioxidotetrahydro-2H-thiopyran-4-yl)-4- | 486.2 | 1.91 | 3, 23 |
| | morpholin-4-yl-1H-pyrazolo[3,4-d]pyrimidin-6- | | | |
| | yl]phenyl}-3-methylurea | | | |
| 870 | tert-butyl (3S)-3-[6-(4-aminophenyl)-4-morpholin-4-yl- | 480.3 | 2.32 | 46 |
| | 1H-pyrazolo[3,4-d]pyrimidin-1-yl]piperidine-1- | | | |
| | carboxylate | | | |
| 871 | tert-butyl (3R)-3-[6-(4-aminophenyl)-4-morpholin-4-yl- | 480.3 | 2.29 | 46 |
| | 1H-pyrazolo[3,4-d]pyrimidin-1-yl]piperidine-1- | | | |
| | carboxylate | | | |
| 872 | tert-butyl (3S)-3-(6-{4- | 537.3 | 2.33 | 46 |
| | [(methylcarbamoyl)amino]phenyl}-4-morpholin-4-yl-1H- | | | |
| | pyrazolo[3,4-d]pyrimidin-1-yl)piperidine-1-carboxylate | | | |
| 873 | 1-methyl-3-(4-{4-morpholin-4-yl-1-[(3S)-piperidin-3-yl]- | 437.2 | 1.65 | 46 |
| | 1H-pyrazolo[3,4-d]pyrimidin-6-yl}phenyl)urea | | | |
| 874 | tert-butyl (3R)-3-(6-{4- | 537.3 | 2.32 | 46 |
| | [(methylcarbamoyl)amino]phenyl}-4-morpholin-4-yl-1H- | | | |
| | pyrazolo[3,4-d]pyrimidin-1-yl)piperidine-1-carboxylate | | | |
| 875 | 1-methyl-3-(4-{4-morpholin-4-yl-1-[(3R)-piperidin-3-yl]- | 437.2 | 1.65 | 46 |
| | 1H-pyrazolo[3,4-d]pyrimidin-6-yl}phenyl)urea | | | |
| 876 | 2,2-dimethylpropyl 4-(6-{4- | 551.3 | 2.41 | 7c, 23 |
| | [(methylcarbamoyl)amino]phenyl}-4-morpholin-4-yl-1H- | | | |
| | pyrazolo[3,4-d]pyrimidin-1-yl)piperidine-1-carboxylate | | | |
| 877 | 2,2-dimethylpropyl 4-(4-morpholin-4-yl-6-{4-[(pyridin-3- | 614.3 | 2.27 | 7c, 23 |
| | ylcarbamoyl)amino]phenyl}-1H-pyrazolo[3,4- | | | |
| | d]pyrimidin-1-yl)piperidine-1-carboxylate | | | |
| 878 | 2-fluoroethyl 4-(6-{4-[(methylcarbamoyl)amino]phenyl}- | 527.2 | 2.1 | 7c, 23 |
| | 4-morpholin-4-yl-1H-pyrazolo[3,4-d]pyrimidin-1- | | | |
| | yl)piperidine-1-carboxylate | | | |
| 879 | 2-fluoroethyl 4-(4-morpholin-4-yl-6-{4-[(pyridin-3- | 590.3 | 1.98 | 7c, 23 |
| | ylcarbamoyl)amino]phenyl}-1H-pyrazolo[3,4- | | | |
| | d]pyrimidin-1-yl)piperidine-1-carboxylate | | | |
| 880 | benzyl 4-(6-{4-[(methylcarbamoyl)amino]phenyl}-4- | 571.3 | 2.34 | 7c, 23 |
| | morpholin-4-yl-1H-pyrazolo[3,4-d]pyrimidin-1- | | | |
| | yl)piperidine-1-carboxylate | | | |
| 881 | benzyl 4-(4-morpholin-4-yl-6-{4-[(pyridin-3- | 634.3 | 2.22 | 7c, 23 |
| | ylcarbamoyl)amino]phenyl}-1H-pyrazolo[3,4- | | | |
| | d]pyrimidin-1-yl)piperidine-1-carboxylate | | | |
| 882 | tert-butyl 4-(6-{4-[(isoxazol-3- | 590.3 | 2.45 | 1, 2, 23 |
| | ylcarbamoyl)amino]phenyl}-4-morpholin-4-yl-1H- | | | |
| | pyrazolo[3,4-d]pyrimidin-1-yl)piperidine-1-carboxylate | | | |
| 883 | tert-butyl 4-[6-(4-{[(3-methylisoxazol-5- | 604.3 | 2.47 | 1, 2, 23 |
| | yl)carbamoyl]amino}phenyl)-4-morpholin-4-yl-1H- | | | |
| | pyrazolo[3,4-d]pyrimidin-1-yl]piperidine-1-carboxylate | | | |
| 884 | tert-butyl 4-(4-morpholin-4-yl-6-{4-[(1,3-thiazol-2- | 606.3 | 2.53 | 1, 2, 23 |
| | ylcarbamoyl)amino]phenyl}-1H-pyrazolo[3,4- | | | |
| | d]pyrimidin-1-yl)piperidine-1-carboxylate | | | |
| 885 | tert-butyl 4-(4-morpholin-4-yl-6-{4-[(pyrazin-2- | 601.3 | 2.54 | 1, 2, 23 |
| | ylcarbamoyl)amino]phenyl}-1H-pyrazolo[3,4- | | | |
| | d]pyrimidin-1-yl)piperidine-1-carboxylate | | | |
| 886 | tert-butyl 4-(4-morpholin-4-yl-6-{4-[(pyrimidin-2- | 601.3 | 2.57 | 1, 2, 23 |
| | ylcarbamoyl)amino]phenyl}-1H-pyrazolo[3,4- | | | |
| | d]pyrimidin-1-yl)piperidine-1-carboxylate | | | |
| 887 | ethyl 4-{6-[4-(1H-imidazol-2-ylamino)phenyl]-4- | 518.3 | 1.92 | 1, 2, 24 |
| | morpholin-4-yl-1H-pyrazolo[3,4-d]pyrimidin-1- | | | |
| | yl}piperidine-1-carboxylate | | | |
| 888 | ethyl 4-(6-{4-[(methylcarbamoyl)amino]phenyl}-4-[(3R)- | 523.3 | 2.12 | 1, 2, 23 |
| | 3-methylmorpholin-4-yl]-1H-pyrazolo[3,4-d]pyrimidin-1- | | | |
| | yl)piperidine-1-carboxylate | | | |
| 889 | ethyl 4-(6-{4-[(ethylcarbamoyl)amino]phenyl}-4-[(3R)-3- | 537.3 | 2.18 | 1, 2, 23 |
| | methylmorpholin-4-yl]-1H-pyrazolo[3,4-d]pyrimidin-1- | | | |
| | yl)piperidine-1-carboxylate | | | |
| 890 | ethyl 4-{6-(4-{[(2-fluoroethyl)carbamoyl]amino}phenyl)- | 555.3 | 2.16 | 1, 2, 23 |
| | 4-[(3R)-3-methylmorpholin-4-yl]-1H-pyrazolo[3,4- | | | |
| | d]pyrimidin-1-yl}piperidine-1-carboxylate | | | |
| 891 | ethyl 4-(4-[(3R)-3-methylmorpholin-4-yl]-6-{4- | 585.3 | 2.37 | 1, 2, 23 |
| | [(phenylcarbamoyl)amino]phenyl}-1H-pyrazolo[3,4- | | | |
| | d]pyrimidin-1-yl)piperidine-1-carboxylate | | | |
| 892 | ethyl 4-(4-[(3R)-3-methylmorpholin-4-yl]-6-{4-[(pyridin- | 586.3 | 2.03 | 1, 2, 23 |
| | 3-ylcarbamoyl)amino]phenyl}-1H-pyrazolo[3,4- | | | |
| | d]pyrimidin-1-yl)piperidine-1-carboxylate | | | |
| 893 | ethyl 4-(4-[(3R)-3-methylmorpholin-4-yl]-6-{4-[(pyridin- | 586.3 | 2 | 1, 2, 23 |
| | 4-ylcarbamoyl)amino]phenyl}-1H-pyrazolo[3,4- | | | |
| | d]pyrimidin-1-yl)piperidine-1-carboxylate | | | |
| 894 | ethyl 4-(6-{4-[(ethylcarbamoyl)amino]phenyl}-4-[(3S)-3- | | | 1, 2, 23 |
| | methylmorpholin-4-yl]-1H-pyrazolo[3,4-d]pyrimidin-1- | | | |
| | yl)piperidine-1-carboxylate | | | |
| 895 | ethyl 4-{6-(4-{[(2-fluoroethyl)carbamoyl]amino}phenyl)- | | | 1, 2, 23 |
| | 4-[(3S)-3-methylmorpholin-4-yl]-1H-pyrazolo[3,4- | | | |
| | d]pyrimidin-1-yl}piperidine-1-carboxylate | | | |
| 896 | ethyl 4-(4-[(3S)-3-methylmorpholin-4-yl]-6-{4- | | | 1, 2, 23 |
| | [(phenylcarbamoyl)amino]phenyl}-1H-pyrazolo[3,4- | | | |
| | d]pyrimidin-1-yl)piperidine-1-carboxylate | | | |
| 897 | ethyl 4-(4-[(3S)-3-methylmorpholin-4-yl]-6-{4-[(pyridin- | | | 1, 2, 23 |
| | 3-ylcarbamoyl)amino]phenyl}-1H-pyrazolo[3,4- | | | |
| | d]pyrimidin-1-yl)piperidine-1-carboxylate | | | |
| 898 | ethyl 4-(4-[(3S)-3-methylmorpholin-4-yl]-6-{4-[(pyridin- | | | 1, 2, 23 |
| | 4-ylcarbamoyl)amino]phenyl}-1H-pyrazolo[3,4- | | | |
| | d]pyrimidin-1-yl)piperidine-1-carboxylate | | | |
| 899 | ethyl 4-{4-[(3S)-3-methylmorpholin-4-yl]-6-(4-{[(4- | | | 1, 2, 23 |
| | morpholin-4-ylphenyl)carbamoyl]amino}phenyl)-1H- | | | |
| | pyrazolo[3,4-d]pyrimidin-1-yl}piperidine-1-carboxylate | | | |
| 900 | methyl 4-(6-{4-[(ethoxycarbonyl)amino]phenyl}-4- | 510.2 | 2.44 | 23 |
| | morpholin-4-yl-1H-pyrazolo[3,4-d]pyrimidin-1- | | | |
| | yl)piperidine-1-carboxylate | | | |
| 901 | methyl 4-[6-(4-{[(2- | 526.2 | 2.15 | 23 |
| | hydroxyethoxy)carbonyl]amino}phenyl)-4-morpholin-4- | | | |
| | yl-1H-pyrazolo[3,4-d]pyrimidin-1-yl]piperidine-1- | | | |
| | carboxylate | | | |
| 902 | methyl 4-[6-(4-{[(2- | 540.2 | 2.35 | 23 |
| | methoxyethoxy)carbonyl]amino}phenyl)-4-morpholin-4- | | | |
| | yl-1H-pyrazolo[3,4-d]pyrimidin-1-yl]piperidine-1- | | | |
| | carboxylate | | | |
| 903 | methyl 4-[6-(4-{[(2- | 525.2 | 1.83 | 23 |
| | aminoethoxy)carbonyl]amino}phenyl)-4-morpholin-4-yl- | | | |
| | 1H-pyrazolo[3,4-d]pyrimidin-1-yl]piperidine-1- | | | |
| | carboxylate | | | |
| 904 | methyl 4-{6-[4-({[2- | 553.3 | 1.9 | 23 |
| | (dimethylamino)ethoxy]carbonyl}amino)phenyl]-4- | | | |
| | morpholin-4-yl-1H-pyrazolo[3,4-d]pyrimidin-1- | | | |
| | yl}piperidine-1-carboxylate | | | |
| 905 | methyl 4-[4-morpholin-4-yl-6-(4-{[(2-pyrrolidin-1- | 579.3 | 1.94 | 23 |
| | ylethoxy)carbonyl]amino}phenyl)-1H-pyrazolo[3,4- | | | |
| | d]pyrimidin-1-yl]piperidine-1-carboxylate | | | |
| 906 | methyl 4-[4-morpholin-4-yl-6-(4-{[(2-morpholin-4- | 595.3 | 1.89 | 23 |
| | ylethoxy)carbonyl]amino}phenyl)-1H-pyrazolo[3,4- | | | |
| | d]pyrimidin-1-yl]piperidine-1-carboxylate | | | |
| 907 | methyl 4-{6-[4-({[2-(4-methylpiperazin-1- | 608.3 | 1.91 | 23 |
| | yl)ethoxy]carbonyl}amino)phenyl]-4-morpholin-4-yl-1H- | | | |
| | pyrazolo[3,4-d]pyrimidin-1-yl}piperidine-1-carboxylate | | | |
| 908 | methyl 4-[4-morpholin-4-yl-6-(4-{[(2,2,2- | 564.2 | 2.43 | 1, 2, 23 |
| | trifluoroethoxy)carbonyl]amino}phenyl)-1H- | | | |
| | pyrazolo[3,4-d]pyrimidin-1-yl]piperidine-1-carboxylate | | | |
| 909 | methyl 4-[6-(4-{[(3- | 540.2 | 2.15 | 1, 2, 23 |
| | hydroxypropoxy)carbonyl]amino}phenyl)-4-morpholin-4- | | | |
| | yl-1H-pyrazolo[3,4-d]pyrimidin-1-yl]piperidine-1- | | | |
| | carboxylate | | | |
| 910 | methyl 4-{6-[4-({[4-(4-methylpiperazin-1- | 655.3 | 1.97 | 1, 2, 23 |
| | yl)phenyl]carbamoyl}amino)phenyl]-4-morpholin-4-yl- | | | |
| | 1H-pyrazolo[3,4-d]pyrimidin-1-yl}piperidine-1- | | | |
| | carboxylate | | | |
| 911 | methyl 4-[4-morpholin-4-yl-6-(4-{[(6-morpholin-4- | 643.3 | 2.01 | 1, 2, 23 |
| | ylpyridin-3-yl)carbamoyl]amino}phenyl)-1H- | | | |
| | pyrazolo[3,4-d]pyrimidin-1-yl]piperidine-1-carboxylate | | | |
| 912 | methyl 4-{6-[4-({[4- | 587.3 | 2.18 | 1, 2, 23 |
| | (hydroxymethyl)phenyl]carbamoyl}amino)phenyl]-4- | | | |
| | morpholin-4-yl-1H-pyrazolo[3,4-d]pyrimidin-1- | | | |
| | yl}piperidine-1-carboxylate | | | |
| 913 | methyl 4-{6-[4-({[4-(2- | 601.3 | 2.22 | 1, 2, 23 |
| | hydroxyethyl)phenyl]carbamoyl}amino)phenyl]-4- | | | |
| | morpholin-4-yl-1H-pyrazolo[3,4-d]pyrimidin-1- | | | |
| | yl}piperidine-1-carboxylate | | | |
| 914 | methyl 4-{4-morpholin-4-yl-6-[4-({[4-(2-pyrrolidin-1- | 654.3 | 2.05 | 1, 2, 23, 25 |
| | ylethyl)phenyl]carbamoyl}amino)phenyl]-1H- | | | |
| | pyrazolo[3,4-d]pyrimidin-1-yl}piperidine-1-carboxylate | | | |
| 915 | methyl 4-{4-morpholin-4-yl-6-[4-({[4-(2-piperidin-1- | 668.4 | 2.09 | 1, 2, 23, 25 |
| | ylethyl)phenyl]carbamoyl}amino)phenyl]-1H- | | | |
| | pyrazolo[3,4-d]pyrimidin-1-yl}piperidine-1-carboxylate | | | |
| 916 | methyl 4-{4-morpholin-4-yl-6-[4-({[4-(2-piperazin-1- | 669.4 | 1.85 | 1, 2, 23, 25 |
| | ylethyl)phenyl]carbamoyl}amino)phenyl]-1H- | | | |
| | pyrazolo[3,4-d]pyrimidin-1-yl}piperidine-1-carboxylate | | | |
| 917 | methyl 4-(6-{4-[({4-[2-(4-methylpiperazin-1- | 683.4 | 1.95 | 1, 2, 23, 25 |
| | yl)ethyl]phenyl}carbamoyl)amino]phenyl}-4-morpholin- | | | |
| | 4-yl-1H-pyrazolo[3,4-d]pyrimidin-1-yl)piperidine-1- | | | |
| | carboxylate | | | |
| 918 | methyl 4-{4-morpholin-4-yl-6-[4-({[4-(2-morpholin-4- | 670.3 | 2 | 1, 2, 23, 25 |
| | ylethyl)phenyl]carbamoyl}amino)phenyl]-1H- | | | |
| | pyrazolo[3,4-d]pyrimidin-1-yl}piperidine-1-carboxylate | | | |
| 919 | methyl 4-{6-[4-({[4-(2-{[2- | 671.4 | 1.79 | 1, 2, 23, 25 |
| | (dimethylamino)ethyl]amino}ethyl)phenyl]carbamoyl}amino) | | | |
| | phenyl]-4-morpholin-4-yl-1H-pyrazolo[3,4- | | | |
| | d]pyrimidin-1-yl}piperidine-1-carboxylate | | | |
| 920 | methyl 4-[6-(4-{[(4-{2-[(2- | 643.3 | 1.77 | 1, 2, 23, 25 |
| | aminoethyl)amino]ethyl}phenyl)carbamoyl]amino}phenyl)- | | | |
| | 4-morpholin-4-yl-1H-pyrazolo[3,4-d]pyrimidin-1- | | | |
| | yl]piperidine-1-carboxylate | | | |
| 921 | methyl 4-[6-(4-{[(4-{2-[(2- | | | 1, 2, 23, 25 |
| | hydroxyethyl)amino]ethyl}phenyl)carbamoyl]amino}phenyl)- | | | |
| | 4-morpholin-4-yl-1H-pyrazolo[3,4-d]pyrimidin-1- | | | |
| | yl]piperidine-1-carboxylate | | | |
| 922 | methyl 4-[6-(4-{[(4-{2-[(2- | | | 1, 2, 23, 25 |
| | methoxyethyl)amino]ethyl}phenyl)carbamoyl]amino}phenyl)- | | | |
| | 4-morpholin-4-yl-1H-pyrazolo[3,4-d]pyrimidin-1- | | | |
| | yl]piperidine-1-carboxylate | | | |
| 923 | 2-hydroxyethyl (4-{4-morpholin-4-yl-1-[1-(2,2,2- | 550.2 | 2.12 | 48 |
| | trifluoroethyl)piperidin-4-yl]-1H-pyrazolo[3,4- | | | |
| | d]pyrimidin-6-yl}phenyl)carbamate | | | |
| 924 | 2-hydroxyethyl (4-{4-morpholin-4-yl-1-[1-(pyridin-3- | 559.3 | 1.68 | 7b, 23 |
| | ylmethyl)piperidin-4-yl]-1H-pyrazolo[3,4-d]pyrimidin-6- | | | |
| | yl}phenyl)carbamate | | | |
| 925 | N-{4-[4-morpholin-4-yl-1-(tetrahydro-2H-pyran-2-yl)- | 423.21398 | | 13a |
| | 1H-pyrazolo[3,4-d]pyrimidin-6-yl]phenyl}acetamide | | | |
| 926 | N-[4-(4-morpholin-4-yl-1H-pyrazolo[3,4-d]pyrimidin-6- | 339.15708 | | 13a |
| | yl)phenyl]acetamide | | | |
| 927 | 1-methyl-3-[4-(4-morpholin-4-yl-1H-pyrazolo[3,4- | 354.168 | | 13a |
| | d]pyrimidin-6-yl)phenyl]urea | | | |
| 928 | 6-(1H-indol-5-yl)-4-morpholin-4-yl-1-(tetrahydro-2H- | 405.20491 | | 13b |
| | pyran-2-yl)-1H-pyrazolo[3,4-d]pyrimidine | | | |
| 929 | 6-(1H-indol-5-yl)-4-morpholin-4-yl-1H-pyrazolo[3,4- | 321.14629 | | 13b |
| | d]pyrimidine | | | |
| 930 | 5-[1-(1-benzylpiperidin-4-yl)-4-morpholin-4-yl-1H- | 472.24541 | | 7a |
| | pyrazolo[3,4-d]pyrimidin-6-yl]pyridin-3-ol | | | |
| 931 | 1-(1-benzylpiperidin-4-yl)-6-[5- | | | 7a, H+ |
| | (methoxymethoxy)pyridin-3-yl]-4-morpholin-4-yl-1H- | | | |
| | pyrazolo[3,4-d]pyrimidine | | | |
| 932 | N-(4-{4-(2-hydroxymorpholin-4-yl)-1-[1-(pyridin-3- | | | |
| | ylmethyl)piperidin-4-yl]-1H-pyrazolo[3,4-d]pyrimidin-6- | | | |
| | yl}phenyl)acetamide | | | |
| 933 | 1-(4-{4-(2-hydroxymorpholin-4-yl)-1-[1-(pyridin-3- | | | |
| | ylmethyl)piperidin-4-yl]-1H-pyrazolo[3,4-d]pyrimidin-6- | | | |
| | yl}phenyl)-3-methylurea | | | |
| 934 | 4-[4-morpholin-4-yl-1-(tetrahydro-2H-pyran-2-yl)-1H- | 381.20514 | | 45, 23 |
| | pyrazolo[3,4-d]pyrimidin-6-yl]aniline | | | |
| 935 | 1-methyl-3-{4-[4-morpholin-4-yl-1-(tetrahydro-2H-pyran- | 438.22554 | | 13c |
| | 2-yl)-1H-pyrazolo[3,4-d]pyrimidin-6-yl]phenyl}urea | | | |
| 936 | {4-[1-(1-benzylpiperidin-4-yl)-4-morpholin-4-yl-1H- | 513.25891 | | 2 |
| | pyrazolo[3,4-d]pyrimidin-6-yl]phenyl}acetic acid | | | |
| 937 | 6-[1-(1-benzylpiperidin-4-yl)-4-morpholin-4-yl-1H- | | | 2 |
| | pyrazolo[3,4-d]pyrimidin-6-yl]quinoline | | | |
| 938 | tert-butyl 4-[6-(4-aminophenyl)-4-morpholin-4-yl-1H- | 480.27325 | | 2 |
| | pyrazolo[3,4-d]pyrimidin-1-yl]piperidine-1-carboxylate | | | |
| 939 | 1-methyl-3-{4-[4-morpholin-4-yl-1-(tetrahydro-2H-pyran- | 438.22597 | | 46 |
| | 4-yl)-1H-pyrazolo[3,4-d]pyrimidin-6-yl]phenyl}urea | | | |
| 940 | 1-[2-chloro-4-(4-morpholin-4-yl-1H-pyrazolo[3,4- | 388.12967 | | 23, 7c, |
| | d]pyrimidin-6-yl)phenyl]-3-methylurea | | | 22 |
| 941 | 1-(4-{4-[(2R,6S)-2,6-dimethylmorpholin-4-yl]-1-[1- | 556.31466 | | 50 |
| | (pyridin-3-ylmethyl)piperidin-4-yl]-1H-pyrazolo[3,4- | | | |
| | d]pyrimidin-6-yl}phenyl)-3-methylurea | | | |
| 942 | 3-{4-[(3R)-3-methylmorpholin-4-yl]-1-phenyl-1H- | 388.17682 | | 57 |
| | pyrazolo[3,4-d]pyrimidin-6-yl}phenol | | | |
| 943 | 3-[4-(2-methylmorpholin-4-yl)-1-phenyl-1H- | 388.1767 | | 58 |
| | pyrazolo[3,4-d]pyrimidin-6-yl]phenol | | | |
| 944 | methyl 4-[6-(4-hydroxyphenyl)-4-morpholin-4-yl-1H- | 439.2091 | | 1, 2, 26 |
| | pyrazolo[3,4-d]pyrimidin-1-yl]piperidine-1-carboxylate | | | |
| 945 | methyl 4-(4-morpholin-4-yl-6-{4- | 558.246 | | 1, 2, 27 |
| | [(phenoxycarbonyl)amino]phenyl}-1H-pyrazolo[3,4- | | | |
| | d]pyrimidin-1-yl)piperidine-1-carboxylate | | | |
| 946 | methyl 4-(6-{4-[(methylcarbamoyl)oxy]phenyl}-4- | 496.2304 | | 1, 2, 26 |
| | morpholin-4-yl-1H-pyrazolo[3,4-d]pyrimidin-1- | | | |
| | yl)piperidine-1-carboxylate | | | |
| 947 | N-{4-[1-(1-isobutyrylpiperidin-4-yl)-4-morpholin-4-yl- | 504.2713 | | 4, 23, 9 |
| | 1H-pyrazolo[3,4-d]pyrimidin-6-yl]phenyl}acrylamide | | | |
| 948 | methyl 4-[6-(4-{[(4- | 576.2363 | | 1, 2, 27 |
| | fluorophenoxy)carbonyl]amino}phenyl)-4-morpholin-4- | | | |
| | yl-1H-pyrazolo[3,4-d]pyrimidin-1-yl]piperidine-1- | | | |
| | carboxylate | | | |
| 949 | methyl 4-[6-(4-{[(Z)- | 582.2563 | | 1, 2, 18 |
| | (cyanoimino)(phenoxy)methyl]amino}phenyl)-4- | | | |
| | morpholin-4-yl-1H-pyrazolo[3,4-d]pyrimidin-1- | | | |
| | yl]piperidine-1-carboxylate | | | |
| 950 | methyl 4-[6-(4-{[(4- | | | 1, 2, 27 |
| | chlorophenoxy)carbonyl]amino}phenyl)-4-morpholin-4- | | | |
| | yl-1H-pyrazolo[3,4-d]pyrimidin-1-yl]piperidine-1- | | | |
| | carboxylate | | | |
| 951 | tert-butyl 4-(6-{4-[(methylsulfamoyl)amino]phenyl}-4- | | | 1, 2, 28 |
| | morpholin-4-yl-1H-pyrazolo[3,4-d]pyrimidin-1- | | | |
| | yl)piperidine-1-carboxylate | | | |
| 952 | tert-butyl 4-[6-(4-{[(6-fluoropyridin-3- | 618.2949 | | 1, 2, 23 |
| | yl)carbamoyl]amino}phenyl)-4-morpholin-4-yl-1H- | | | |
| | pyrazolo[3,4-d]pyrimidin-1-yl]piperidine-1-carboxylate | | | |
| 953 | tert-butyl 4-{6-[4-({[4- | | | 1, 2, 23 |
| | (hydroxymethyl)phenyl]carbamoyl}amino)phenyl]-4- | | | |
| | morpholin-4-yl-1H-pyrazolo[3,4-d]pyrimidin-1- | | | |
| | yl}piperidine-1-carboxylate | | | |
| 954 | tert-butyl 4-{6-[4-({[4-(2- | 643.3351 | | 1, 2, 23 |
| | hydroxyethyl)phenyl]carbamoyl}amino)phenyl]-4- | | | |
| | morpholin-4-yl-1H-pyrazolo[3,4-d]pyrimidin-1- | | | |
| | yl}piperidine-1-carboxylate | | | |
| 955 | 1-(4-{3-[3-(dimethylamino)prop-1-yn-1-yl]-1-ethyl-4- | | | 45, 23 |
| | morpholin-4-yl-1Hpyrazolo[3,4-d]pyrimidin-6- | | | |
| | yl}phenyl)-3-methylurea | | | |
| 956 | 1-{4-[1-ethyl-3-(3-hydroxyprop-1-yn-1-yl)-4-morpholin- | | | 45, 23 |
| | 4-yl-1H-pyrazolo[3,4-d]pyrimidin-6-yl]phenyl}-3- | | | |
| | pyridin-3-ylurea | | | |
| 957 | 4-{4-[6-(1H-indol-5-yl)-4-morpholin-4-yl-1H- | | | 7c |
| | pyrazolo[3,4-d]pyrimidin-1-yl]piperidin-1-yl}-N,N- | | | |
| | dimethyl-4-oxobut-2-en-1-amine | | | |
| 958 | N2,N2-dimethyl-N-[4-(4-morpholin-4-yl-1H- | | | 43 |
| | pyrazolo[3,4-d]pyrimidin-6-yl)phenyl]glycinamide | | | |
| 959 | methyl (4-{1-[1-(4-fluorobenzyl)piperidin-4-yl]-4- | | | 23, 3 |
| | morpholin-4-yl-1Hpyrazolo[3,4-d]pyrimidin-6- | | | |
| | yl}phenyl)carbamate | | | |
| 960 | N-(4-{4-(2-hydroxymorpholin-4-yl)-1-[1-(pyridin-3- | 529 | | |
| | ylmethyl)piperidin-4-yl]-1Hpyrazolo[3,4-d]pyrimidin-6- | | | |
| | yl}phenyl)acetamide | | | |
| 961 | 1-(4-{4-(2-hydroxymorpholin-4-yl)-1-[1-(pyridin-3- | 544 | | |
| | ylmethyl)piperidin-4-yl]-1Hpyrazolo[3,4-d]pyrimidin-6- | | | |
| | yl}phenyl)-3-methylurea | | | |
| 962 | 3-[4-(1,4-oxazepan-4-yl)-1-phenyl-1H-pyrazolo[3,4- | 388.17682 | | 59 |
| | d]pyrimidin-6-yl]phenol | | | |
| 963 | 3-(1-phenyl-4-thiomorpholin-4-yl-1H-pyrazolo[3,4- | 390.1 | 2.54 | 60 |
| | d]pyrimidin-6-yl)phenol | | | |
| 964 | 3-(3-fluoro-4-morpholin-4-yl-1-phenyl-1H-pyrazolo[3,4- | 392.2 | | 61 |
| | d]pyrimidin-6-yl)phenol | | | |

¹High resolution mass spectrometry

Biological Evaluation—mTOR Kinase Assay Methods

Human mTOR assays (See Toral-Barza, et al Biochem Biophys. Res. Commun. Jun. 24, 2005;332(1):304-10) with purified enzyme are performed in 96-well plates by DELFIA format as follows. Enzymes are first diluted in kinase assay buffer (10 mM HEPES (pH 7.4), 50 mM NaCl, 50 mM β-glycerophosphate, 10 mM MnCl₂, 0.5 mM DTT, 0.25 mM microcystin LR, and 100 mg/mL BSA). To each well, 12 μL of the diluted enzyme is mixed briefly with 0.5 μL test inhibitor or control vehicle dimethylsulfoxide (DMSO). The kinase reaction is initiated by adding 12.5 μL kinase assay buffer containing ATP and His6-S6K to give a final reaction volume of 25 μL containing 800 ng/mL FLAG-TOR, 100 mM ATP and 1.25 mM His6-S6K. The reaction plate is incubated for 2 hours (linear at 1-6 hours) at room temperature with gentle shaking and then terminated by adding 25 μL Stop buffer (20 mM HEPES (pH 7.4), 20 mM EDTA, 20 mM EGTA). The DELFIA detection of the phosphorylated (Thr-389) His6-S6K is performed at room temperature using a monoclonal anti-P(T389)-p70S6K antibody (1A5, Cell Signaling) labeled with Europium-N1-ITC (Eu) (10.4 Eu per antibody, PerkinElmer). The DELFIA Assay buffer and Enhancement solution can be purchased from PerkinElmer. 45 μL of the terminated kinase reaction mixture is transferred to a MaxiSorp plate (Nunc) containing 55 μL PBS. The His6-S6K is allowed to attach for 2 hours after which the wells are aspirated and washed once with PBS. 100 μL of DELFIA Assay buffer with 40 ng/mL Eu-P(T389)-S6K antibody is added. The antibody binding is continued for 1 hour with gentle agitation. The wells are then aspirated and washed 4 times with PBS containing 0.05% Tween-20 (PBST). 100 μL of DELFIA Enhancement solution is added to each well and the plates are read in a PerkinElmer Victor model plate reader. Data obtained is used to calculate enzymatic activity and enzyme inhibition by potential inhibitors.

### Fluorescence Polarization Assay for PI3K

This assay is used to determine the IC₅₀ of compounds of the present invention as it identifies inhibitors of P13 kinase by measuring inhibition.

### Materials

Reaction Buffer: 20 mM HEPES, pH 7.5, 2 mM MgCl2, 0.05% CHAPS; and 0.01% BME (added fresh) Stop/Detection Buffer: 100 mM HEPES, pH 7.5, 4 mM EDTA, 0.05% CHAPS; ATP 20 mM in water; PIP2 (diC8, cat# P-4508) 1 mM in water (MW=856.5); GST-GRP 1.75 mg/mL or 1.4 mg/mL in 10% glycerol; Red detector (TAMRA) 2.5 μM; Plate: Nunc 384 well black polypropylene fluorescence plate.

### Methods

The assay is run by placing 5 μL of diluted enzyme per well, then 5 μL of diluted compound (or 9.5 μL enzyme then 0.5 μL compound in DMSO) is added and mixed. Then, 10 μL substrate is added to start the reaction. The samples are incubated 30-60 minutes, then the reaction is stopped by adding 20 μL stop/detector mix.

PI3K is diluted with reaction buffer (e.g., 5 μL or 7.5 μL PI3K into 620 μL reaction buffer), and 5 μL of diluted enzyme is used per well. 5 μL reaction buffer or drug diluted in buffer (e.g., 4 μL/100 so final DMSO is 1% in reaction) is added to each. Pipetting up and down mixes the samples. Alternatively, the enzyme can be diluted to 1215 μL. In this case 9.8 μL is added per well and 0.2 μL compound is added in DMSO.

To prepare 1 mL of substrate solution, 955 μL reaction buffer, 40 μL PIP2, and 2.5 μL ATP are mixed. 10 μL of substrate is added to each well to start the reaction. This results in 20 μM PIP2, and 25 μM ATP per reaction.

Stop/detector mix is prepared by mixing 4 μL Red detector and 1.6 μL or 2.0 μL GST-GRP with 1 mL Stop buffer, which results in 10 nM probe and 70 nM GST-GRP). 20 μL of the stop/detector mix is added to each well to stop the reaction. The plates are read after 30-90 minutes keeping the red probe solutions dark.

For the zero time point, stop/detector mix is added to the enzyme just before adding substrate. For an extra control, stop/detector mix is added to buffer (no enzyme) and substrate or to just buffer (no substrate).

Pooled PI3K preparations had a protein concentration of 0.25 mg/mL. The recommended reaction has 0.06 μL per 20 μL (0.015 μg/20 μL) or 0.01 125 μg/15 μL or 0.75 μg/mL.

Plates are read on machines with filters for Tamra. The units are mP with no enzyme controls reading app 190-220 mP units. Fully active enzyme reduces fluorescence polarization down to 70-100 mP after 30 minutes. An active compound raises the mP values halfway to control or to 120-150 mP units.

### In Vitro Cell Culture Growth Assay Methods:

Human tumor cell lines used include prostate lines LNCap and PC3MM2, breast lines MDA468, MCF7, renal line HTB44 (A498), colon line HCT116, and ovarian line OVCAR_{3.} Cells were plated in 96-well culture plates. One day following plating, the inhibitors were added to cells. Three days after drug treatment, viable cell densities were determined by metabolic conversion (by viable cells) of the dye MTS, a well established cell proliferation assay. The assays were performed using an assay kit purchased from Promega Corp. (Madison, Wis.) following the protocol supplied with the kit. The MTS assay results were read in a 96-well plate reader by measuring absorbance at 490 nm. The effect of each treatment was calculated as percent of control growth relative to the vehicle-treated cells grown in the same culture plate. The drug concentration that conferred 50% inhibition of growth was determined as IC₅₀ (μg/ml). Table 2 shows the results of the described biological assays.

TABLE 2

| | mTOR IC₅₀ | PI3Kα | LNCap IC₅₀ | MDA468 IC₅₀ |
|---|---|---|---|---|
| Compound | (nM) | IC₅₀ (nM) | (μM) | (μM) |
| 1 | | 813 | | |
| 2 | 655.0 | | | |
| 3 | 37.0 | 915 | | |
| 4 | 2800.0 | 5500 | 20 | 38.0 |
| 5 | 240.0 | 440 | 2.7 | 20.0 |
| 6 | 190.0 | 1353 | 2.4 | 11.0 |
| 7 | 290.0 | 10000 | 20 | 38.0 |
| 8 | 730.0 | 31 | 0.22 | 1.9 |
| 9 | 78.0 | 36 | 0.37 | 2.2 |
| 10 | 215.0 | 69 | 0.6 | 3.2 |
| 11 | 49.0 | 279 | 4.7 | 5.0 |
| 12 | 645.0 | | 0.75 | 12.0 |
| 13 | 20000.0 | 34 | 0.27 | 1.5 |
| 14 | 48.5 | 20 | 0.11 | 0.6 |
| 15 | 17.5 | 55 | 0.074 | 0.6 |
| 16 | 17.5 | 47 | 0.05 | 0.4 |
| 17 | 9.6 | 107 | 0.31 | 2.0 |
| 18 | 85.5 | 22 | 0.22 | 1.3 |
| 19 | 17.5 | 11 | 0.45 | 1.7 |
| 20 | 38.0 | 21 | 0.13 | 0.7 |
| 21 | 23.0 | 98 | 0.5 | 1.4 |
| 22 | 29.0 | 31 | 0.3 | 0.9 |
| 23 | 19.0 | 63 | 0.15 | 0.8 |
| 24 | 14.0 | 86 | 0.2 | 0.7 |
| 25 | 6.2 | 50 | 0.14 | 0.7 |
| 26 | 4.4 | 68 | 1.3 | 4.0 |
| 27 | 25.0 | 41 | 0.55 | 2.4 |
| 28 | 13.5 | 33 | 0.22 | 1.1 |
| 29 | 6.5 | 59 | 0.4 | 1.2 |
| 30 | 36.5 | 69 | 0.5 | 1.4 |
| 31 | 48.7 | 3566 | 7.2 | 9.0 |
| 32 | 1215.0 | 1436 | 8.8 | 10.0 |
| 33 | 2900.0 | 1163 | | |
| 34 | 1200.0 | 1112 | 2 | 2.2 |
| 35 | 130.0 | 8204 | | |
| 36 | 720.0 | 10000 | | |
| 37 | 20000.0 | 10000 | | |
| 38 | 6300.0 | 5870 | 0.38 | 1.1 |
| 39 | 4.0 | 1962 | 0.5 | 1.4 |
| 40 | 8.8 | 3746 | 0.8 | 0.9 |
| 41 | 47.0 | 2901 | 4 | 9.0 |
| 42 | 155.0 | 9742 | 3 | 6.0 |
| 43 | 44.5 | 4000 | 8 | 19.0 |
| 44 | 545.0 | 11000 | 1.7 | 1.4 |
| 45 | 380.0 | 1008 | 1.4 | 1.9 |
| 46 | 88.3 | 1310 | | |
| 47 | 170.0 | 1962 | 0.49 | 1.4 |
| 48 | 8.8 | 780 | 0.59 | 2.3 |
| 49 | 23.5 | 1272 | 2.1 | 5.8 |
| 50 | 230.0 | 2294 | 0.7 | 2.4 |
| 51 | 40.5 | 1096 | 0.34 | 1.7 |
| 52 | 11.5 | 1432 | 1.5 | 5.8 |
| 53 | 94.0 | 846 | 0.22 | 3.9 |
| 54 | 29.0 | 1746 | 0.53 | 2.6 |
| 55 | 22.5 | 686 | 2.5 | 3.0 |
| 56 | 116.0 | 425 | 2 | 2.7 |
| 57 | 117.5 | 1698 | 3 | 4.0 |
| 58 | 140.0 | 428 | 1.2 | 1.5 |
| 59 | 120.0 | 239 | 0.5 | 2.6 |
| 60 | 145.0 | 989 | 2 | 5.5 |
| 61 | 300.0 | 9132 | 7 | 11.0 |
| 62 | 4600.0 | 9187 | 6.2 | 12.0 |
| 63 | 10500.0 | 2555 | 6.2 | 10.0 |
| 64 | 6900.0 | 244 | 3.2 | 7.0 |
| 65 | 695.0 | 2766 | 9.5 | 17.0 |
| 66 | 7300.0 | 4111 | 4.8 | 4.7 |
| 67 | 4600.0 | 2908 | 10 | 12.0 |
| 68 | 257.5 | 1210 | 4.9 | 8.3 |
| 69 | 99.0 | 10500 | 3.1 | 4.0 |
| 70 | 7600.0 | 3871 | 3 | 15.0 |
| 71 | 4600.0 | 10000 | 10 | 13.0 |
| 72 | 10500.0 | 520 | 9.5 | 6.0 |
| 73 | 380.0 | 8768 | 40 | 45.0 |
| 74 | 18000.0 | 8573 | 15 | 21.0 |
| 75 | 20000.0 | 10000 | 60 | 60.0 |
| 76 | 14750.0 | 522 | 0.9 | 1.0 |
| 77 | 20.5 | 2862 | 5.1 | 9.0 |
| 78 | 1230.0 | 2005 | 8 | 12.0 |
| 79 | 2450.0 | 1368 | 4.5 | 10.0 |
| 80 | 200.0 | 420 | 0.45 | 2.3 |
| 81 | 12.9 | 3676 | 60 | 45.0 |
| 82 | 165.0 | 877 | 13 | 29.0 |
| 83 | 1650.0 | 2415 | 4 | 7.0 |
| 84 | 1750.0 | 4198 | 12 | 12.0 |
| 85 | 2550.0 | 2282 | 8 | 9.0 |
| 86 | 1850.0 | 4662 | 7.2 | 6.5 |
| 87 | 12400.0 | 780 | 23 | 60.0 |
| 88 | 20000.0 | 2187 | 60 | 58.0 |
| 89 | 1425.0 | 5190 | 15 | 20.0 |
| 90 | 575.0 | 918 | 1.2 | 3.2 |
| 91 | 9.1 | 35 | 0.41 | 2.5 |
| 92 | 4.1 | 4645 | 8 | 2.5 |
| 93 | 375.0 | 337 | 1.1 | 1.2 |
| 94 | 50.0 | 1458 | 1.9 | 4.2 |
| 95 | 210.0 | 2455 | 0.7 | 2.1 |
| 96 | 22.0 | 449 | 1.3 | 2.8 |
| 97 | 165.0 | 18 | 0.31 | 1.3 |
| 98 | 14.3 | 754 | 60 | 33.0 |
| 99 | 165.0 | 332 | 3.5 | 5.0 |
| 100 | 122.5 | 359 | 60 | 60.0 |
| 101 | 211.8 | 494 | 10 | 22.0 |
| 102 | 20.3 | 320 | 3.2 | 7.5 |
| 103 | 812.5 | 580 | 3.9 | 5.0 |
| 104 | 762.5 | 244 | 3.7 | 3.3 |
| 105 | 1075.0 | 3084 | 60 | 60.0 |
| 106 | 1315.0 | 290 | 3 | 11.0 |
| 107 | 72.5 | 436 | 3.5 | 10.0 |
| 108 | 111.0 | 137 | 60 | 60.0 |
| 109 | 16.3 | 248 | 45 | 30.0 |
| 110 | 21.3 | 231 | 2 | 5.5 |
| 111 | 26.8 | 1009 | 12 | 23.0 |
| 112 | 135.5 | 1905 | 15 | 60.0 |
| 113 | 52.8 | 2169 | 22 | 23.0 |
| 114 | 1520.0 | 393 | 15 | 27.0 |
| 115 | 1072.5 | 357 | 5.2 | 9.0 |
| 116 | 195.0 | 111 | 20 | 60.0 |
| 117 | 103.8 | 699 | 60 | 60.0 |
| 118 | 352.5 | 122 | 7 | 20.0 |
| 119 | 40.0 | 1162 | 60 | 60.0 |
| 120 | 1625.0 | 737 | 0.7 | 2.0 |
| 121 | 78.0 | 2128 | 0.6 | 2.0 |
| 122 | 44.5 | 132 | 0.7 | 1.3 |
| 123 | 24.5 | 1296 | 0.45 | 1.8 |
| 124 | 25.5 | 843 | 0.83 | 1.7 |
| 125 | 180.0 | 9750 | 4.4 | 7.3 |
| 126 | 2400.0 | 60 | 3.3 | 13.0 |
| 127 | 1550.0 | 6517 | 5 | 18.0 |
| 128 | 1750.0 | 384 | 4.8 | 60.0 |
| 129 | 102.0 | 59 | 0.41 | 1.3 |
| 130 | 9.6 | 822 | 60 | 60.0 |
| 131 | 725.0 | 1099 | 60 | 60.0 |
| 132 | 845.0 | 311 | 0.3 | 1.2 |
| 133 | 34.5 | 223 | | |
| 134 | 117.8 | 64 | 1.2 | 1.3 |
| 135 | 23.5 | 325 | 3.4 | 1.2 |
| 136 | 61.5 | 49 | 1 | 0.4 |
| 137 | 12.0 | 100 | 2.8 | 1.2 |
| 138 | 96.5 | 303 | 4.9 | 6.5 |
| 139 | 34.0 | 134 | 1.5 | 60.0 |
| 140 | 12.5 | 131 | 2.9 | 1.3 |
| 141 | 79.0 | 209 | 3 | 3.8 |
| 142 | 76.5 | 1495 | 0.32 | 2.2 |
| 143 | 15.5 | 703 | 0.18 | 1.7 |
| 144 | 24.0 | 627 | 2.4 | 3.8 |
| 145 | 535.0 | 583 | 5.5 | 21.0 |
| 146 | 195.0 | 457 | 1.7 | 4.0 |
| 147 | 575.0 | 350 | 5.6 | 22.0 |
| 148 | 375.0 | 162 | 60 | 60.0 |
| 149 | 58.5 | 793 | 4.5 | 40.0 |
| 150 | 480.0 | 1953 | 0.3 | 2.1 |
| 151 | 275.0 | 1268 | 0.52 | 2.3 |
| 152 | 305.0 | 1133 | 0.6 | 2.3 |
| 153 | 13.0 | 460 | 2 | 6.5 |
| 154 | 32.5 | 486 | 0.5 | 3.0 |
| 155 | 11.5 | 107 | 0.1 | 1.0 |
| 156 | 17.0 | 450 | 3.3 | 35.0 |
| 157 | 460.0 | 1029 | 33 | 60.0 |
| 158 | 3150.0 | 404 | 0.07 | 1.0 |
| 159 | 7.1 | 878 | 0.63 | 2.5 |
| 160 | 4.5 | 6000 | 2.8 | 15.0 |
| 161 | 1140.0 | 3515 | 14 | 60.0 |
| 162 | 250.0 | 1853 | 33 | 60.0 |
| 163 | 5550.0 | 60 | 1.5 | 6.0 |
| 164 | 285.0 | 230 | 5.7 | 20.0 |
| 165 | 455.0 | 176 | 3 | 12.0 |
| 166 | 245.0 | 45 | 5.8 | 15.0 |
| 167 | 400.0 | 890 | 3 | 3.1 |
| 168 | 540.0 | 10000 | 3.4 | 10.0 |
| 169 | 1350.0 | 341 | 2 | 12.0 |
| 170 | 255.0 | 10000 | 3.3 | 12.0 |
| 171 | 545.0 | 552 | 0.73 | 2.9 |
| 172 | 16.0 | 366 | 1 | 1.7 |
| 173 | 2.5 | 262 | 1.2 | 2.7 |
| 174 | 3.8 | 304 | 1 | 2.8 |
| 175 | 24.5 | 462 | 1.1 | 2.0 |
| 176 | 16.0 | 352 | 1 | 2.6 |
| 177 | 42.5 | 885 | 1.3 | 3.1 |
| 178 | 44.5 | 509 | 0.55 | 2.1 |
| 179 | 17.0 | 649 | 0.6 | 1.8 |
| 180 | 23.0 | 405 | 2 | 5.8 |
| 181 | 110.5 | 2476 | 1.5 | 5.5 |
| 182 | 615.0 | 3929 | 5 | 4.1 |
| 183 | 455.0 | 1169 | 11 | 13.0 |
| 184 | 145.0 | 3078 | 60 | 60.0 |
| 185 | 250.0 | 180 | 1.2 | 3.9 |
| 186 | 16.5 | 3425 | 4 | 3.9 |
| 187 | 295.0 | 7765 | 35 | 40.0 |
| 188 | 4850.0 | 2999 | 7 | 19.0 |
| 189 | 625.0 | 765 | 3.2 | 8.3 |
| 190 | 485.0 | 1319 | 6.2 | 6.0 |
| 191 | 3750.0 | 18 | 0.08 | 0.4 |
| 192 | 20.5 | 7810 | 16 | 40.0 |
| 193 | 8000.0 | 8250 | 10 | 12.0 |
| 194 | 1900.0 | 1063 | 0.95 | 3.0 |
| 195 | 1.4 | 1690 | 0.45 | 1.0 |
| 196 | 0.9 | 1278 | 0.75 | 3.0 |
| 197 | 11.0 | 3057 | 2.5 | 3.8 |
| 198 | 14.0 | 1649 | 0.7 | 4.0 |
| 199 | 7.0 | 2936 | 0.45 | 1.8 |
| 200 | 9.4 | 2393 | 0.75 | 2.0 |
| 201 | 7.3 | 2022 | 0.74 | 3.0 |
| 202 | 16.3 | 2096 | 0.4 | 2.0 |
| 203 | 7.6 | 1155 | 1.05 | 3.0 |
| 204 | 10.3 | 26 | 2.5 | 8.0 |
| 205 | 0.5 | 589 | 2.4 | 8.0 |
| 206 | 75.5 | 14 | 0.001 | 0.1 |
| 207 | 0.5 | 659 | 3 | 5.2 |
| 208 | 46.0 | 3793 | 5.8 | 11.0 |
| 209 | 95.5 | 801 | 0.213 | 1.5 |
| 210 | 4.6 | 108 | 0.2 | 1.1 |
| 211 | 3.4 | 23 | 0.17 | 0.8 |
| 212 | 9.0 | 106 | 0.37 | 1.4 |
| 213 | 2.2 | 514 | 0.355 | 1.2 |
| 214 | 1.6 | 109 | 0.57 | 1.5 |
| 215 | 3.7 | 37 | 0.65 | 1.3 |
| 216 | 4.7 | 446 | 0.4 | 1.5 |
| 217 | 1.0 | 1399 | 1.2 | 1.9 |
| 218 | 60.5 | 41 | 0.007 | 0.0 |
| 219 | 0.4 | 548 | 0.15 | 0.6 |
| 220 | 0.8 | 404 | 0.066 | 1.0 |
| 221 | 7.1 | 878 | 0.625 | 2.5 |
| 222 | 4.5 | 801 | 0.213 | 1.5 |
| 223 | 4.6 | 514 | 0.355 | 1.2 |
| 224 | 1.6 | 1647 | 2.5 | 10.5 |
| 225 | 74.5 | 41 | 0.007 | 0.0 |
| 226 | 0.4 | 548 | 0.15 | 0.6 |
| 227 | 0.8 | 9024 | 6 | 60.0 |
| 228 | 6800.0 | 1916 | 5.8 | 6.8 |
| 229 | 2250.0 | 2171 | 6.8 | 12.0 |
| 230 | 2025.0 | 6000 | 60 | 22.0 |
| 231 | 120.0 | 684 | 1.4 | 4.5 |
| 232 | 105.5 | 720 | 1.4 | 4.0 |
| 233 | 170.0 | 443 | 3.7 | 8.5 |
| 234 | 50.0 | 481 | 1 | 2.0 |
| 235 | 34.5 | 4915 | 11 | 40.0 |
| 236 | 335.0 | 105 | 1.3 | 1.2 |
| 237 | 3.7 | 2420 | 4.2 | 1.0 |
| 238 | 57.5 | 3749 | 2.5 | 2.9 |
| 239 | 260.0 | 3087 | 0.78 | 3.1 |
| 240 | 40.5 | 2669 | 3.8 | 3.5 |
| 241 | 420.0 | 5000 | 2.4 | 4.2 |
| 242 | 205.0 | 692 | 0.7 | 3.0 |
| 243 | 58.0 | 535 | 1.3 | 4.3 |
| 244 | 38.0 | 3302 | 0.75 | 2.1 |
| 245 | 10.4 | 2659 | 0.8 | 2.8 |
| 246 | 24.0 | 3990 | 1.8 | 3.0 |
| 247 | 260.0 | 7293 | 2.5 | 3.0 |
| 248 | 430.0 | 939 | 3.5 | 9.0 |
| 249 | 98.5 | 2003 | | |
| 250 | 2275.0 | 37 | | |
| 251 | 37.5 | 2804 | 1 | 3.0 |
| 252 | 87.5 | 83 | 0.032 | 0.3 |
| 253 | 2.7 | 257 | 0.03 | 0.4 |
| 254 | 0.8 | 341 | 0.22 | 1.5 |
| 255 | 2.8 | 4114 | 5 | 11.0 |
| 256 | 155.0 | 270 | 0.56 | 3.0 |
| 257 | 4.8 | 17 | 0.2 | 1.1 |
| 258 | 2.4 | 533 | 2.3 | 12.0 |
| 259 | 390.0 | 2138 | 1 | 20.0 |
| 260 | 31.5 | 7029 | 4.5 | 60.0 |
| 261 | 95.5 | 55 | 0.18 | 0.6 |
| 262 | 0.8 | 385 | 0.7 | 3.0 |
| 263 | 3.1 | 268 | 0.25 | 1.8 |
| 264 | 5.0 | 119 | 0.28 | 1.0 |
| 265 | 4.5 | 86 | 0.027 | 0.1 |
| 266 | 0.3 | 401 | 0.8 | 3.7 |
| 267 | 23.3 | 14 | 2 | 2.7 |
| 268 | 3.2 | 29 | 0.095 | 0.6 |
| 269 | 65.0 | 364 | 0.08 | 0.7 |
| 270 | 2.8 | 753 | 0.6 | 3.2 |
| 271 | 15.3 | 158 | 0.65 | 2.0 |
| 272 | 152.5 | 708 | 1.1 | 1.2 |
| 273 | 125.0 | 8321 | 1.95 | 60.0 |
| 274 | 2900.0 | 2546 | 2 | 12.0 |
| 275 | 34.0 | 9000 | 12 | 40.0 |
| 276 | 895.0 | 6467 | 11 | 60.0 |
| 277 | 3350.0 | 440 | 60 | 60.0 |
| 278 | 98.0 | 1167 | 7.2 | 60.0 |
| 279 | 130.0 | 612 | 3 | 12.0 |
| 280 | 61.5 | 601 | 3.4 | 7.0 |
| 281 | 86.0 | 2271 | 7 | 18.0 |
| 282 | 1450.0 | 128 | 9 | 11.0 |
| 283 | 140.0 | 203 | 1.5 | 13.0 |
| 284 | 210.0 | 885 | 5.3 | 22.0 |
| 285 | 465.0 | 1613 | 5.3 | 18.0 |
| 286 | 8.6 | 317 | 0.5 | 0.2 |
| 287 | 15.8 | 273 | 0.68 | 0.2 |
| 288 | 13.6 | 234 | 0.027 | 0.0 |
| 289 | 1.4 | 590 | 0.32 | 1.3 |
| 290 | 10.4 | 1935 | 3.5 | 8.0 |
| 291 | 84.0 | 4226 | 3 | 10.0 |
| 292 | 68.5 | 171 | 5.2 | 8.0 |
| 293 | 7.3 | 211 | 0.48 | 1.1 |
| 294 | 9.6 | 251 | 0.19 | 0.9 |
| 295 | 0.8 | 404 | 1.8 | 5.0 |
| 296 | 18.5 | 896 | 3 | 60.0 |
| 297 | 57.0 | 51 | 0.2 | 3.0 |
| 299 | 1.9 | 1903 | 1.1 | 4.0 |
| 300 | 115.0 | 13 | 0.2 | 0.2 |
| 301 | 10.9 | 179 | 3 | 1.8 |
| 302 | 0.6 | 32 | 0.2 | 0.0 |
| 303 | 0.1 | 541 | 0.63 | 0.5 |
| 304 | 0.2 | 220 | 7.5 | 9.0 |
| 305 | 0.3 | 604 | 10.1 | 7.2 |
| 306 | 3.6 | 398 | 10.05 | 14.0 |
| 307 | 1.4 | 434 | 0.06 | 0.2 |
| 308 | 0.3 | 1814 | 0.8 | 3.0 |
| 309 | 6.8 | 155 | 0.9 | 7.5 |
| 310 | 6.5 | 199 | 0.1 | 1.1 |
| 311 | 17.0 | 323 | 0.05 | 1.1 |
| 312 | 2.6 | 173 | 0.4 | 3.9 |
| 313 | 17.0 | 82 | 0.2 | 0.2 |
| 314 | 1.9 | 20 | 0.027 | 0.0 |
| 315 | 0.2 | 338 | 0.027 | 0.0 |
| 316 | 0.8 | 163 | 0.5 | 0.6 |
| 317 | 0.7 | 259 | 1.1 | 1.5 |
| 318 | 10.7 | 187 | 0.027 | 0.0 |
| 319 | 0.5 | 1005 | 0.5 | 3.0 |
| 320 | 23.0 | 2052 | 1.1 | 7.2 |
| 321 | 21.0 | 74 | 0.027 | 0.1 |
| 322 | 0.3 | 56 | 0.027 | 0.2 |
| 323 | 0.5 | 39 | 0.054 | 0.2 |
| 324 | 0.6 | 4207 | 4 | 11.0 |
| 325 | 64.5 | 71 | 0.05 | 0.4 |
| 326 | 2.1 | 30 | 1 | 3.5 |
| 327 | 0.7 | 4394 | 4 | 5.0 |
| 328 | 195.0 | 198 | 5 | 11.5 |
| 329 | 0.8 | 216 | 0.25 | 0.9 |
| 330 | 0.4 | 33 | 0.027 | 0.7 |
| 331 | 0.7 | 65 | 0.027 | 0.5 |
| 332 | 0.4 | 66 | 0.6 | 1.0 |
| 333 | 0.5 | 23 | 0.027 | 0.2 |
| 334 | 0.5 | 71 | 0.4 | 0.7 |
| 335 | 1.2 | 30 | 3 | 2.5 |
| 336 | 1.5 | 70 | 0.027 | 0.0 |
| 337 | 0.1 | 80 | 0.027 | 0.3 |
| 338 | 0.7 | 23 | 0.5 | 1.3 |
| 339 | 1.4 | 11 | 0.031 | 0.2 |
| 340 | 0.5 | 13 | 0.028 | 0.1 |
| 341 | 0.2 | 91 | 0.33 | 1.8 |
| 342 | 0.9 | 29 | 1.2 | 1.1 |
| 343 | 9.2 | 14 | 0.027 | 0.0 |
| 344 | 0.1 | 15 | 0.027 | 0.0 |
| 345 | 0.1 | 261 | 0.095 | 0.7 |
| 346 | 0.4 | 45 | 5 | 9.0 |
| 347 | 6.3 | 1653 | 2 | 4.0 |
| 348 | 115.0 | 199 | 0.08 | 0.8 |
| 349 | 2.4 | 485 | 0.23 | 2.5 |
| 350 | 6.3 | 246 | 0.027 | 0.2 |
| 351 | 1.0 | 41 | 0.07 | 0.4 |
| 352 | 0.6 | 35 | 0.027 | 0.1 |
| 353 | 0.4 | 1264 | 33 | 40.0 |
| 354 | 123.5 | 80 | 0.027 | 0.9 |
| 355 | 0.2 | 79 | 0.027 | 0.0 |
| 356 | 0.2 | 93 | 0.027 | 0.1 |
| 357 | 0.2 | 89 | 0.027 | 0.1 |
| 358 | 0.2 | 53 | 0.027 | 0.1 |
| 359 | 0.4 | 76 | 0.045 | 0.2 |
| 360 | 0.5 | 35 | 0.052 | 0.2 |
| 361 | 1.0 | 108 | 0.027 | 0.1 |
| 362 | 0.8 | 42 | 0.052 | 0.4 |
| 363 | 1.2 | 55 | 0.045 | 0.4 |
| 364 | 0.7 | 75 | 0.035 | 0.3 |
| 365 | 0.5 | 82 | 0.027 | 0.3 |
| 366 | 0.4 | 73 | 0.027 | 0.0 |
| 367 | 0.5 | 43 | 0.027 | 0.1 |
| 368 | 0.7 | 89 | 0.15 | 0.2 |
| 369 | 1.6 | 466 | 60 | 60.0 |
| 370 | 5950.0 | 3134 | 5 | 11.0 |
| 371 | 190.0 | 4058 | 9.3 | 10.0 |
| 372 | 170.0 | 424 | 0.1 | 0.9 |
| 373 | 2.6 | 55 | 0.04 | 0.2 |
| 374 | 0.4 | 619 | 0.14 | 1.3 |
| 375 | 3.5 | 10 | 0.034 | 0.1 |
| 376 | 0.3 | 359 | 1.8 | 6.3 |
| 377 | 40.0 | 124 | 0.07 | 0.5 |
| 378 | 1.9 | 16 | 0.027 | 0.1 |
| 379 | 0.2 | 35 | 0.027 | 0.0 |
| 380 | 0.2 | 454 | 0.027 | 0.0 |
| 381 | 0.1 | 355 | 0.027 | 0.0 |
| 382 | 0.1 | 85 | 0.17 | 0.2 |
| 383 | 0.4 | 1579 | 0.9 | 4.3 |
| 384 | 5.2 | 19 | 0.042 | 0.1 |
| 385 | 0.2 | 229 | 0.8 | 0.7 |
| 386 | 0.2 | 48 | 1.2 | 1.4 |
| 387 | 1.3 | 109 | 0.035 | 0.1 |
| 388 | 0.6 | 47 | 0.027 | 0.1 |
| 389 | 0.5 | 31 | 0.027 | 0.1 |
| 390 | 0.4 | 90 | 0.045 | 0.1 |
| 391 | 0.3 | 82 | 0.07 | 0.2 |
| 392 | 0.7 | 153 | 0.11 | 0.6 |
| 393 | 0.7 | 72 | 0.095 | 0.6 |
| 394 | 0.5 | 18 | 0.06 | 0.3 |
| 395 | 1.0 | 42 | 0.1 | 0.5 |
| 396 | 0.8 | 35 | 0.038 | 0.2 |
| 397 | 0.5 | 28 | 0.14 | 0.4 |
| 398 | 1.2 | 57 | 0.15 | 0.6 |
| 399 | 2.9 | 37 | 0.06 | 0.3 |
| 400 | 0.8 | 43 | 0.07 | 0.5 |
| 401 | 1.0 | 211 | 1 | 3.8 |
| 402 | 21.5 | 63 | 0.027 | 0.5 |
| 403 | 2.4 | 45 | 0.103 | 0.4 |
| 404 | 0.8 | 9 | 0.027 | 0.1 |
| 405 | 0.2 | 438 | 0.027 | 0.1 |
| 406 | 0.8 | 260 | 1.4 | 1.1 |
| 407 | 4.6 | 774 | 1.7 | 3.8 |
| 408 | 6.4 | 916 | 0.89 | 3.2 |
| 409 | 6.3 | 363 | 0.8 | 2.5 |
| 410 | 1.4 | 593 | 2.2 | 1.3 |
| 411 | 4.5 | 344 | 20.05 | 12.0 |
| 412 | 3.6 | 382 | 3 | 6.0 |
| 413 | 2.0 | 15 | 0.035 | 0.0 |
| 414 | 0.1 | 18 | 0.04 | 0.1 |
| 415 | 0.1 | 357 | 0.37 | 0.7 |
| 416 | 0.3 | 1486 | 10 | 13.0 |
| 417 | 1255.0 | 160 | 0.7 | 0.7 |
| 418 | 0.6 | 124 | 0.12 | 0.2 |
| 419 | 0.2 | 231 | 0.32 | 0.4 |
| 420 | 0.4 | 119 | 0.33 | 0.3 |
| 421 | 0.5 | 100 | 0.25 | 0.3 |
| 422 | 0.6 | 198 | 0.32 | 0.4 |
| 423 | 0.7 | 83 | 0.11 | 0.2 |
| 424 | 0.4 | 84 | 0.15 | 0.4 |
| 425 | 0.7 | 100 | 0.08 | 0.5 |
| 426 | 0.3 | 112 | 0.06 | 0.7 |
| 427 | 1.3 | 34 | 0.027 | 0.2 |
| 428 | 1.8 | 30 | 0.027 | 0.2 |
| 429 | 1.3 | 51 | 0.04 | 0.2 |
| 430 | 1.3 | 42 | 0.052 | 0.2 |
| 431 | 1.9 | 25 | 0.027 | 0.1 |
| 432 | 2.5 | 440 | 20 | 60.0 |
| 433 | 58.5 | 1031 | 60 | 60.0 |
| 434 | 160.0 | 6660 | 39 | 60.0 |
| 435 | 800.0 | 4037 | 45 | 60.0 |
| 436 | 800.0 | 4994 | 9.8 | 25.0 |
| 437 | 385.0 | 1455 | 60 | 60.0 |
| 438 | 135.0 | 1050 | 7 | 60.0 |
| 439 | 145.0 | 1000 | 60 | 60.0 |
| 440 | 112.5 | 2502 | | |
| 441 | 3.4 | 1924 | 0.2 | 0.6 |
| 442 | 2.7 | 1013 | 0.15 | 0.5 |
| 443 | 2.2 | 1137 | 0.25 | 0.5 |
| 444 | 1.6 | 778 | 0.25 | 0.8 |
| 445 | 1.4 | 989 | 0.58 | 1.6 |
| 446 | 4.4 | 888 | 0.35 | 0.9 |
| 447 | 1.7 | 999 | 0.6 | 1.2 |
| 448 | 7.0 | 725 | 0.9 | 1.7 |
| 449 | 12.5 | 800 | 0.5 | 1.2 |
| 450 | 3.0 | 750 | 1.2 | 2.6 |
| 451 | 17.5 | 389 | 2 | 9.0 |
| 452 | 36.5 | 1831 | 0.6 | 2.2 |
| 453 | 13.5 | 1198 | 0.9 | 6.0 |
| 454 | 13.5 | 619 | 0.74 | 2.0 |
| 455 | 10.3 | 786 | 2.5 | 5.2 |
| 456 | 19.5 | 1431 | 1.3 | 3.2 |
| 457 | 13.0 | 585 | 0.5 | 1.8 |
| 458 | 13.5 | 1980 | 0.93 | 1.2 |
| 459 | 17.0 | 1068 | 1.7 | 3.0 |
| 460 | 38.5 | 4032 | 4.9 | 14.0 |
| 461 | 155.0 | 887 | 1.9 | 1.5 |
| 462 | 3.9 | 1615 | 0.9 | 0.9 |
| 463 | 2.9 | 1727 | 1.2 | 1.2 |
| 464 | 2.6 | 3623 | 5.2 | 22.0 |
| 465 | 375.0 | 1543 | 1.05 | 2.7 |
| 466 | 5.2 | 3106 | 1.05 | 3.3 |
| 467 | 10.3 | 2985 | 1.2 | 2.2 |
| 468 | 7.0 | 3564 | 1 | 2.2 |
| 469 | 8.5 | 173 | 0.17 | 0.6 |
| 470 | 2.1 | 187 | 0.056 | 0.3 |
| 471 | 9.2 | 2880 | 4 | 7.8 |
| 472 | 54.0 | 225 | 0.8 | 1.2 |
| 473 | 2.1 | 209 | 0.2 | 0.5 |
| 474 | 1.1 | 287 | 1.01 | 1.4 |
| 475 | 2.3 | 148 | 0.15 | 0.3 |
| 476 | 1.4 | 100 | 0.031 | 0.1 |
| 477 | 0.5 | 149 | 2.2 | 1.8 |
| 478 | 2.0 | 9500 | 9 | 22.0 |
| 479 | 650.0 | 138 | 0.13 | 1.0 |
| 480 | 15.0 | 42 | 0.8 | 1.2 |
| 481 | 6.8 | 9024 | 6 | 60 |
| 482 | 2.25 | 1916 | 5.8 | 6.8 |
| 483 | 2.025 | 2171 | 6.8 | 12 |
| 484 | 0.12 | 6000 | >60.00000 | 22 |
| 485 | 0.1055 | 684 | 1.4 | 4.5 |
| 486 | 0.17 | 720 | 1.4 | 4 |
| 487 | 0.0345 | 480 | 1 | 2 |
| 488 | 0.26 | 3749 | 2.5 | 2.9 |
| 489 | 0.0405 | 3087 | 0.78 | 3.1 |
| 490 | 0.42 | 2669 | 3.8 | 3.5 |
| 491 | 0.205 | 5000 | 2.4 | 4.2 |
| 492 | 0.058 | 692 | 0.7 | 3 |
| 493 | 0.038 | 535 | 1.3 | 4.3 |
| 494 | 0.0104 | 3302 | 0.75 | 2.05 |
| 495 | 0.024 | 2659 | 0.8 | 2.8 |
| 496 | 0.26 | 3990 | 1.8 | 3 |
| 497 | 0.43 | 7293 | 2.5 | 3 |
| 498 | 0.002733 | 82 | 0.032 | 0.28 |
| 499 | 0.00079 | 256 | 0.03 | 0.35 |
| 500 | 0.0028 | 341 | 0.22 | 1.5 |
| 501 | 0.155 | 4114 | 5 | 11 |
| 502 | 0.0048 | 270 | 0.56 | 3 |
| 503 | 0.00235 | 16 | 0.2 | 1.05 |
| 504 | 0.39 | 533 | 2.3 | 12 |
| 505 | 0.0315 | 2138 | 1 | 20 |
| 506 | 0.0955 | 7028 | 4.5 | >60.0000 |
| 507 | 0.000825 | 55 | 0.18 | 0.6 |
| 508 | 0.0031 | 384 | 0.7 | 3 |
| 509 | 0.004967 | 268 | 0.25 | 1.8 |
| 510 | 0.000305 | 86 | <0.02700 | 0.05 |
| 511 | 0.02325 | 400 | 0.8 | 3.7 |
| 512 | 0.00315 | 14 | 2 | 2.7 |
| 513 | 0.065 | 29 | 0.095 | 0.6 |
| 514 | 0.002775 | 364 | 0.08 | 0.65 |
| 515 | 0.01525 | 753 | 0.6 | 3.2 |
| 516 | 0.1525 | 158 | 0.65 | 2 |
| 517 | 0.125 | 708 | 1.1 | 1.2 |
| 518 | 0.0875 | 2804 | 1 | 3 |
| 519 | 2.9 | 8321 | 1.95 | >60.0000 |
| 520 | 0.034 | 2546 | 2 | 12 |
| 521 | 0.895 | 9000 | 12 | 40 |
| 522 | 3.35 | 6467 | 11 | >60.0000 |
| 523 | 0.098 | 440 | >60.00000 | >60.0000 |
| 524 | 0.13 | 1166 | 7.2 | 60 |
| 525 | 0.0615 | 612 | 3 | 12 |
| 526 | 0.086 | 601 | 3.4 | 7 |
| 527 | 1.45 | 2270 | 7 | 18 |
| 528 | 0.14 | 128 | 9 | 11 |
| 529 | 0.21 | 202 | 1.5 | 13 |
| 530 | 0.465 | 885 | 5.3 | 22 |
| 531 | 0.0086 | 1612 | 5.3 | 18 |
| 532 | 0.01575 | 317 | 0.5 | 0.16 |
| 533 | 0.0136 | 273 | 0.68 | 0.16 |
| 534 | 0.0014 | 234 | <0.02740 | 0.0274 |
| 535 | 0.01035 | 590 | 0.32 | 1.3 |
| 536 | 0.084 | 1934 | 3.5 | 8 |
| 537 | 0.0685 | 4226 | 3 | 10 |
| 538 | 0.0073 | 170 | 5.2 | 8 |
| 539 | 0.0096 | 211 | 0.48 | 1.1 |
| 540 | 0.000835 | 250 | 0.19 | 0.9 |
| 541 | 0.0185 | 404 | 1.8 | 5 |
| 542 | 0.057 | 896 | 3 | 60 |
| 543 | 0.0019 | 50 | 0.2 | 3 |
| 544 | 0.115 | 1903 | 1.1 | 4 |
| 545 | 0.0109 | 13 | 0.2 | 0.15 |
| 546 | 0.000585 | 178 | 3 | 1.8 |
| 547 | <0.000155 | 32 | 0.2 | 0.027 |
| 548 | 0.00024 | 541 | 0.63 | 0.52 |
| 549 | 0.00032 | 220 | 7.5 | 9 |
| 550 | 0.0036 | 604 | 10.1 | 7.2 |
| 551 | 0.00135 | 398 | 10.05 | 14 |
| 552 | 0.000295 | 434 | 0.06 | 0.18 |
| 553 | 0.0068 | 1814 | 0.8 | 3 |
| 554 | 0.0065 | 155 | 0.9 | 7.5 |
| 555 | 0.017 | 198 | 0.1 | 1.1 |
| 556 | 0.0026 | 323 | 0.05 | 1.05 |
| 557 | 0.017 | 173 | 0.4 | 3.9 |
| 558 | 0.001875 | 82 | 0.2 | 0.2 |
| 559 | 0.000235 | 20 | <0.02700 | <0.0270 |
| 560 | 0.000825 | 338 | <0.02700 | 0.027 |
| 561 | 0.0007 | 162 | 0.5 | 0.6 |
| 562 | 0.01065 | 259 | 1.1 | 1.5 |
| 563 | 0.000545 | 187 | <0.02700 | 0.027 |
| 564 | 0.023 | 1005 | 0.5 | 3 |
| 565 | 0.021 | 2052 | 1.1 | 7.2 |
| 566 | <0.000655 | 74 | <0.02700 | 0.085 |
| 567 | <0.000730 | 56 | 0.027 | 0.18 |
| 568 | <0.000815 | 39 | 0.054 | 0.24 |
| 569 | 0.00205 | 71 | 0.05 | 0.4 |
| 570 | 0.00073 | 30 | 1 | 3.5 |
| 571 | 0.195 | 4394 | 4 | 5 |
| 572 | 0.00075 | 198 | 5 | 11.5 |
| 573 | 0.000385 | 216 | 0.25 | 0.9 |
| 574 | 0.00065 | 33 | <0.02700 | 0.7 |
| 575 | 0.0004 | 64 | <0.02700 | 0.5 |
| 576 | 0.000455 | 66 | 0.6 | 1 |
| 577 | 0.00052 | 22 | <0.02700 | 0.18 |
| 578 | 0.00115 | 71 | 0.4 | 0.7 |
| 579 | 0.00145 | 30 | 3 | 2.5 |
| 580 | 0.000155 | 63 | <0.02700 | 0.027 |
| 581 | | | | |
| 582 | 0.000415 | 80 | 0.027 | 0.21 |
| 583 | 0.00095 | | 0.027 | 0.45 |
| 584 | 0.001365 | 22 | 0.5 | 1.3 |
| 585 | ND | ND | ND | ND |
| 586 | 0.000485 | 11 | 0.031 | 0.22 |
| 587 | 0.00021 | 12 | 0.028 | 0.12 |
| 588 | 0.00092 | 90 | 0.33 | 1.8 |
| 589 | 0.00915 | 29 | 1.2 | 1.1 |
| 590 | 0.000143 | 14 | <0.02700 | <0.0270 |
| 591 | 0.000116 | 15 | 0.027 | <0.0270 |
| 592 | 0.000375 | 260 | 0.095 | 0.7 |
| 593 | 0.00625 | 45 | 5 | 9 |
| 594 | 0.115 | 1653 | 2 | 4 |
| 595 | 0.00235 | 199 | 0.08 | 0.8 |
| 596 | 0.0063 | 485 | 0.23 | 2.5 |
| 597 | 0.001005 | 246 | 0.027 | 0.18 |
| 598 | 0.00064 | 41 | 0.07 | 0.36 |
| 599 | 0.000447 | 34 | <0.02700 | 0.082 |
| 600 | 0.00019 | 80 | <0.02700 | 0.9 |
| 601 | 0.000155 | 79 | <0.02700 | <0.0270 |
| 602 | 0.00015 | 93 | <0.02700 | 0.07 |
| 603 | 0.000215 | 88 | <0.02700 | 0.08 |
| 604 | 0.00037 | 53 | <0.02700 | 0.05 |
| 605 | 0.000475 | 76 | 0.045 | 0.16 |
| 606 | 0.00099 | 34 | 0.052 | 0.18 |
| 607 | 0.000775 | 108 | 0.027 | 0.09 |
| 608 | 0.00117 | 42 | 0.052 | 0.4 |
| 609 | 0.000745 | 54 | 0.045 | 0.35 |
| 610 | 0.00053 | 75 | 0.035 | 0.34 |
| 611 | 0.00041 | 82 | <0.02700 | 0.33 |
| 612 | 0.00047 | 72 | <0.02700 | 0.027 |
| 613 | 0.00067 | 42 | <0.02700 | 0.085 |
| 614 | 0.00155 | 89 | 0.15 | 0.22 |
| 615 | 0.19 | 3134 | 5 | 11 |
| 616 | 0.17 | 4058 | 9.3 | 10 |
| 617 | 0.0026 | 424 | 0.1 | 0.9 |
| 618 | 0.00044 | 54 | 0.04 | 0.15 |
| 619 | 0.00345 | 619 | 0.14 | 1.3 |
| 620 | 0.00025 | 10 | 0.034 | 0.13 |
| 621 | 0.04 | 359 | 1.8 | 6.3 |
| 622 | 0.00185 | 124 | 0.07 | 0.5 |
| 623 | 0.00016 | 16 | <0.02700 | 0.05 |
| 624 | 0.000155 | 34 | <0.02700 | <0.0270 |
| 625 | 0.000145 | 454 | <0.02700 | 0.027 |
| 626 | 0.00013 | 355 | <0.02700 | 0.038 |
| 627 | 0.00035 | 84 | 0.17 | 0.16 |
| 628 | 0.00515 | 1579 | 0.9 | 4.3 |
| 629 | 0.000225 | 19 | 0.042 | 0.07 |
| 630 | 0.000165 | 229 | 0.8 | 0.7 |
| 631 | 0.00128 | 48 | 1.2 | 1.4 |
| 632 | 0.000645 | 108 | 0.035 | 0.12 |
| 633 | 0.00047 | 47 | <0.02700 | 0.048 |
| 634 | 0.00038 | 30 | 0.027 | 0.08 |
| 635 | 0.00031 | 90 | 0.045 | 0.11 |
| 636 | 0.00065 | 82 | 0.07 | 0.22 |
| 637 | 0.000695 | 153 | 0.11 | 0.55 |
| 638 | 0.000545 | 72 | 0.095 | 0.56 |
| 639 | 0.00104 | 18 | 0.06 | 0.3 |
| 640 | 0.000805 | 42 | 0.1 | 0.5 |
| 641 | 0.000465 | 35 | 0.038 | 0.18 |
| 642 | 0.0012 | 28 | 0.14 | 0.42 |
| 643 | 0.00285 | 57 | 0.15 | 0.6 |
| 644 | 0.00084 | 36 | 0.06 | 0.32 |
| 645 | 0.00095 | 43 | 0.07 | 0.5 |
| 646 | 0.0215 | 211 | 1 | 3.8 |
| 647 | 0.00235 | 63 | 0.027 | 0.5 |
| 648 | 0.00024 | 8 | <0.02700 | 0.06 |
| 649 | 0.000805 | 438 | <0.02700 | 0.085 |
| 650 | 0.0046 | 260 | 1.4 | 1.1 |
| 651 | 0.00635 | 774 | 1.7 | 3.8 |
| 652 | 0.00625 | 916 | 0.89 | 3.2 |
| 653 | 0.0014 | 363 | 0.8 | 2.5 |
| 654 | 0.0045 | 592 | 2.2 | 1.3 |
| 655 | 0.00355 | 344 | 20.05 | 12 |
| 656 | 0.002 | 382 | 3 | 6 |
| 657 | 0.00012 | 14 | 0.035 | 0.04 |
| 658 | 0.000114 | 18 | 0.04 | 0.075 |
| 659 | 0.000325 | 357 | 0.37 | 0.7 |
| 660 | 1.255 | 1486 | 10 | 13 |
| 661 | 0.00063 | 160 | 0.7 | 0.72 |
| 662 | 0.00018 | 124 | 0.12 | 0.22 |
| 663 | 0.000385 | 230 | 0.32 | 0.43 |
| 664 | 0.000485 | 118 | 0.33 | 0.27 |
| 665 | 0.000635 | 100 | 0.25 | 0.27 |
| 666 | 0.000705 | 198 | 0.32 | 0.4 |
| 667 | 0.000435 | 83 | 0.11 | 0.15 |
| 668 | 0.00071 | 84 | 0.15 | 0.41 |
| 669 | 0.00032 | 100 | 0.08 | 0.46 |
| 670 | 0.001345 | 112 | 0.06 | 0.7 |
| 671 | 0.00175 | 34 | <0.02700 | 0.21 |
| 672 | 0.00125 | 30 | 0.027 | 0.15 |
| 673 | 0.0013 | 50 | 0.04 | 0.18 |
| 674 | 0.0019 | 42 | 0.052 | 0.19 |
| 675 | 0.0025 | 25 | <0.02700 | 0.11 |
| 676 | 0.0585 | 440 | 20 | >60.0000 |
| 677 | 0.16 | 1030 | >60.00000 | >60.0000 |
| 678 | >0.800000 | 6660 | 39 | >60.0000 |
| 679 | >0.800000 | 4037 | 45 | >60.0000 |
| 680 | 0.385 | 4994 | 9.8 | 25 |
| 681 | 0.135 | 1455 | >60.00000 | >60.0000 |
| 682 | 0.145 | 1050 | 7 | 60 |
| 683 | 0.1125 | 1000 | >60.00000 | >60.0000 |
| 684 | 0.00335 | 2502 | | |
| 685 | 0.00265 | 1924 | 0.2 | 0.58 |
| 686 | 0.0022 | 1013 | 0.15 | 0.5 |
| 687 | 0.00155 | 1137 | 0.25 | 0.48 |
| 688 | 0.00135 | 778 | 0.25 | 0.75 |
| 689 | 0.0044 | 989 | 0.58 | 1.6 |
| 690 | 0.00165 | 888 | 0.35 | 0.9 |
| 691 | 0.007 | 999 | 0.6 | 1.2 |
| 692 | 0.0125 | 725 | 0.9 | 1.7 |
| 693 | 0.00295 | 800 | 0.5 | 1.2 |
| 694 | 0.0175 | 750 | 1.2 | 2.6 |
| 695 | 0.0365 | 388 | 2 | 9 |
| 696 | 0.0135 | 1831 | 0.6 | 2.2 |
| 697 | 0.0135 | 1198 | 0.9 | 6 |
| 698 | 0.01025 | 619 | 0.74 | 2 |
| 699 | 0.0195 | 786 | 2.5 | 5.2 |
| 700 | 0.013 | 1431 | 1.3 | 3.2 |
| 701 | 0.0135 | 585 | 0.5 | 1.8 |
| 702 | 0.017 | 1980 | 0.93 | 1.2 |
| 703 | 0.0385 | 1068 | 1.7 | 3 |
| 704 | 0.155 | 4032 | 4.9 | 14 |
| 705 | 0.00385 | 887 | 1.9 | 1.5 |
| 706 | 0.0029 | 1615 | 0.9 | 0.9 |
| 707 | 0.00255 | 1727 | 1.2 | 1.2 |
| 708 | 0.375 | 3623 | 5.2 | 22 |
| 709 | 0.00515 | 1543 | 1.05 | 2.7 |
| 710 | 0.01025 | 3106 | 1.05 | 3.3 |
| 711 | 0.00695 | 2985 | 1.2 | 2.2 |
| 712 | 0.0085 | 3564 | 1 | 2.2 |
| 713 | 0.0021 | 173 | 0.17 | 0.6 |
| 714 | 0.0092 | 186 | 0.056 | 0.32 |
| 715 | 0.054 | 2880 | 4 | 7.8 |
| 716 | 0.0021 | 224 | 0.8 | 1.2 |
| 717 | 0.00106 | 209 | 0.2 | 0.48 |
| 718 | 0.00225 | 287 | 1.01 | 1.4 |
| 719 | 0.00135 | 148 | 0.15 | 0.25 |
| 720 | 0.000355 | 100 | 0.027 | 0.095 |
| 721 | 0.00032 | | 0.038 | 0.08 |
| 722 | 0.002 | 148 | 2.2 | 1.8 |
| 723 | 0.65 | 9500 | 9 | 22 |
| 724 | 0.015 | 138 | 0.13 | 1 |
| 725 | 0.00495 | 42 | 0.8 | 1.15 |
| 726 | 0.025 | 98 | 2.7 | 8 |
| 727 | 0.21 | 11000 | 55 | >60.0000 |
| 728 | 0.0085 | 40 | 0.24 | 0.745 |
| 729 | 0.001 | 194 | 0.225 | 0.6 |
| 730 | 0.0089 | 364 | 0.42 | 1.8 |
| 731 | 0.000315 | 490 | <0.02700 | 0.07 |
| 732 | 0.000605 | 951 | 0.0274 | 0.09 |
| 733 | 0.00049 | 402 | 0.8 | 0.58 |
| 734 | 0.00045 | 660 | 0.04 | 0.15 |
| 735 | 0.00034 | 16 | 0.0272 | 0.08 |
| 736 | 0.000558 | 1148 | 0.565 | 17 |
| 737 | 0.000195 | 34 | <0.02700 | <0.0270 |
| 738 | 0.0003 | 18 | <0.02700 | <0.0270 |
| 739 | 0.00375 | 1893 | 0.4 | 1.75 |
| 740 | 0.00575 | | 0.31 | 1.3 |
| 741 | 0.082 | 6115 | 3.4 | 12 |
| 742 | 0.00051 | 153 | 0.05 | 0.22 |
| 743 | 0.015 | 212 | 1.8 | 2.2 |
| 744 | 0.089 | 1050 | 3.5 | 10.2 |
| 745 | 0.0091 | 550 | 1.3 | 2.9 |
| 746 | 0.0089 | 370 | 29 | 30 |
| 747 | 0.014 | 782 | 3.3 | 6.66 |
| 748 | 0.00345 | 53 | 0.08 | 0.39 |
| 749 | 0.745 | 4230 | 12 | 30 |
| 750 | 0.000185 | 221 | 0.033 | 0.08 |
| 751 | 0.000345 | 106 | 0.034 | 0.11 |
| 752 | 0.00022 | 188 | 0.028 | 0.053 |
| 753 | 0.000315 | 100 | 0.047 | 0.11 |
| 754 | 0.000405 | 72 | 0.13 | 0.27 |
| 755 | 0.00021 | 16 | 0.06 | 0.16 |
| 756 | 0.000635 | 174 | 0.5 | 0.5 |
| 757 | 0.00155 | 124 | 2 | 1.1 |
| 758 | 0.00104 | 98 | 0.37 | 0.3 |
| 759 | 0.00091 | 78 | 0.4 | 0.48 |
| 760 | 0.000245 | 42 | 0.17 | 0.05 |
| 761 | 0.000535 | 72 | 0.05 | 0.12 |
| 762 | 0.00055 | 87 | 0.05 | 0.17 |
| 763 | 0.0016 | 154 | 0.27 | 0.5 |
| 764 | 0.0005 | 738 | 0.115 | 0.37 |
| 765 | 0.000495 | 786 | 0.11 | 0.36 |
| 766 | 0.000295 | 364 | 0.18 | 0.41 |
| 767 | 0.00064 | 898 | 0.16 | 0.7 |
| 768 | 0.0014 | 74 | 0.205 | 0.71 |
| 769 | 0.0028 | 2191 | 3.5 | 7 |
| 770 | 0.000375 | 56 | 0.105 | 0.26 |
| 771 | 0.00047 | 59 | 0.07 | 0.19 |
| 772 | 0.0073 | 5028 | 1.3 | 5.5 |
| 773 | 0.00255 | 306 | 0.62 | 0.39 |
| 774 | 0.0051 | 436 | 2.3 | 1.05 |
| 775 | 0.00265 | 170 | 26 | 12 |
| 776 | 0.00345 | 442 | 1.1 | 1.1 |
| 777 | 0.00084 | 19 | 0.13 | 0.14 |
| 778 | 0.00125 | 154 | 1.3 | 1.6 |
| 779 | 0.000625 | 26 | 2.8 | 0.9 |
| 780 | 0.00053 | 16 | 2.2 | 1.5 |
| 781 | 0.013 | 1740 | 1.8 | 7.9 |
| 782 | 0.00094 | 766 | 0.06 | 0.3 |
| 783 | 0.000765 | 1117 | 0.06 | 0.39 |
| 784 | 0.00109 | 380 | 0.1 | 1.05 |
| 785 | 0.00235 | 890 | 3.8 | 12 |
| 786 | 0.000565 | 48 | 0.08 | 1.1 |
| 787 | 0.00034 | 40 | 0.027 | 0.14 |
| 788 | 0.00145 | 734 | 0.12 | 1.6 |
| 789 | 0.00069 | 18 | 0.1 | 0.9 |
| 790 | 0.0006 | 314 | 0.06 | 0.15 |
| 791 | 0.000665 | 136 | 0.22 | 0.26 |
| 792 | 0.00023 | 10 | <0.02700 | <0.0270 |
| 793 | 0.00087 | 76 | 1.01 | 1.2 |
| 794 | 0.000755 | 126 | 0.14 | 0.5 |
| 795 | 0.00066 | 158 | 0.102 | 0.42 |
| 796 | 0.24 | 1564 | 22 | 30.05 |
| 797 | 0.00103 | 155 | 0.103 | 0.3 |
| 798 | 0.0049 | 438 | >60.00000 | >60.0000 |
| 799 | 0.00375 | 72 | 0.14 | 0.51 |
| 800 | 0.000715 | 119 | 0.14 | 0.26 |
| 801 | 0.00046 | 50 | 0.031 | 0.12 |
| 802 | 0.0019 | 181 | 10 | 5.5 |
| 803 | 0.00205 | 173 | 1 | 1 |
| 804 | 0.00057 | 140 | 0.7 | 0.8 |
| 805 | 0.0046 | 2672 | 0.51 | 0.8 |
| 806 | 0.0047 | 4554 | 0.7 | 0.9 |
| 807 | 0.014 | 4451 | 1.4 | 8 |
| 808 | 0.0064 | 2624 | 0.43 | 0.69 |
| 809 | 0.0205 | 2104 | 2.3 | 4.5 |
| 810 | 0.0135 | 2231 | 1.3 | 3 |
| 811 | 0.037 | 1346 | 1.9 | 4.6 |
| 812 | 0.00825 | 688 | 2.4 | 3 |
| 813 | 0.019 | 1961 | 2.25 | 4.4 |
| 814 | 0.00035 | 34 | <0.02700 | <0.0270 |
| 815 | 0.00067 | 3812 | 1.5 | 5 |
| 816 | 0.00019 | 60 | <0.02700 | <0.0270 |
| 817 | 0.000175 | 40 | <0.02700 | <0.0270 |
| 818 | 0.000205 | 445 | 0.32 | 0.25 |
| 819 | 0.000205 | 794 | <0.02700 | 0.07 |
| 820 | 0.000175 | 704 | <0.02700 | 0.13 |
| 821 | 0.000485 | 1119 | <0.02700 | 0.14 |
| 822 | 0.00085 | 1225 | 0.12 | 0.5 |
| 823 | 0.001095 | 571 | 3.5 | 0.32 |
| 824 | 0.0017 | 2032 | 0.11 | 0.7 |
| 825 | 0.00125 | 1675 | 0.15 | 0.62 |
| 826 | 0.000545 | 29 | 0.03 | 0.11 |
| 827 | 0.00105 | 2258 | 0.6 | 16 |
| 828 | 0.000225 | 50 | 0.12 | 0.33 |
| 829 | 0.000315 | 38 | 0.025 | 0.029 |
| 830 | 0.0485 | 12000 | 1.8 | 16 |
| 831 | 0.000595 | 236 | 0.05 | 0.39 |
| 832 | 0.016 | 8000 | 2 | 3.7 |
| 833 | 0.00032 | 1207 | 0.04 | 0.25 |
| 834 | 0.00034 | 450 | 0.22 | 0.18 |
| 835 | 0.00049 | 1782 | 0.05 | 0.32 |
| 836 | 0.00069 | 56 | 0.058 | 0.17 |
| 837 | 0.00165 | 12000 | 2.5 | 4.6 |
| 838 | 0.00029 | 74 | 0.028 | 0.15 |
| 839 | 0.0003 | 37 | 0.027 | 0.068 |
| 840 | 0.041 | 3551 | 3.2 | 10 |
| 841 | 0.0019 | 268 | 0.15 | 0.5 |
| 842 | 0.00245 | 721 | 0.07 | 1 |
| 843 | 0.0024 | 1382 | 0.08 | 0.9 |
| 844 | 0.00235 | 896 | 0.059 | 0.7 |
| 845 | 0.0026 | 402 | 1.1 | 1.6 |
| 846 | 0.000625 | 81 | 0.058 | 0.5 |
| 847 | 0.00365 | 8319 | 13 | 15 |
| 848 | 0.0021 | 60 | 0.062 | 0.72 |
| 849 | 0.00155 | 24 | 0.095 | 0.3 |
| 850 | 0.000365 | 38 | <0.02700 | 0.12 |
| 851 | 0.000375 | 22 | <0.02700 | 0.11 |
| 852 | 0.00057 | 577 | 0.08 | 0.51 |
| 853 | 0.00175 | 824 | 0.13 | 1 |
| 854 | 0.000565 | 600 | 0.027 | 0.33 |
| 855 | 0.0003 | 186 | 0.43 | 0.33 |
| 856 | 0.000535 | 770 | 0.09 | 0.51 |
| 857 | 0.00515 | 2771 | 1.6 | 5 |
| 858 | 0.0048 | 2150 | 0.8 | 3.2 |
| 859 | 0.000285 | 28 | 0.027 | 0.078 |
| 860 | 0.0011 | 38 | 0.12 | 0.39 |
| 861 | 0.00145 | 250 | 0.11 | 0.58 |
| 862 | 0.00053 | 107 | <0.02300 | <0.0885 |
| 863 | 0.000665 | 266 | 0.17 | 0.68 |
| 864 | 0.000255 | 116 | 0.0274 | 0.098 |
| 865 | 0.00365 | 2946 | 0.68 | 4.1 |
| 866 | 0.0455 | 12000 | 12 | 30 |
| 867 | 0.00031 | 47 | <0.01850 | <0.0685 |
| 868 | 0.000815 | 80 | 1.2 | 0.72 |
| 869 | 0.001025 | 74 | 0.54 | 0.59 |
| 870 | 0.49 | 549 | 7.6 | 26 |
| 871 | 0.665 | 4302 | 7.5 | 2.3 |
| 872 | 0.00405 | 169 | 0.27 | 1 |
| 873 | 0.0043 | 74 | 0.41 | 0.73 |
| 874 | 0.01185 | 294 | 0.4 | 1.1 |
| 875 | 0.01145 | 46 | 1.1 | 1.25 |
| 876 | 0.001035 | 87 | 0.11 | 0.35 |
| 877 | 0.0013 | 32 | 0.12 | 0.4 |
| 878 | 0.000335 | 54 | 0.04 | 0.12 |
| 879 | 0.000104 | 12 | 0.0039 | 0.034 |
| 880 | 0.00058 | 20 | 0.03 | 0.22 |
| 881 | 0.00155 | 7 | 0.06 | 0.3 |
| 882 | 0.000595 | 113 | 0.07 | 0.49 |
| 883 | 0.0021 | 160 | 0.27 | 1.3 |
| 884 | 0.0016 | 264 | 0.26 | 1.6 |
| 885 | 0.0029 | 716 | 7.3 | 24 |
| 886 | 0.0145 | 1234 | 2.6 | 6.7 |
| 887 | 0.00505 | 168 | 1.2 | 2.3 |
| 888 | 0.00104 | 1985 | 0.01 | 0.22 |
| 889 | 0.000875 | 4362 | 0.019 | 0.6 |
| 890 | 0.00124 | 4546 | 0.012 | 0.5 |
| 891 | 0.00105 | 590 | 0.02 | 0.21 |
| 892 | 0.000155 | 453 | 0.0008 | 0.07 |
| 893 | 0.00028 | 517 | 0.005 | 0.18 |
| 894 | 0.0018 | 553 | 0.1 | 0.96 |
| 895 | 0.0018 | 521 | 0.1 | 0.95 |
| 896 | 0.0025 | 19 | 0.15 | 0.8 |
| 897 | 0.00056 | 28 | 0.04 | 0.32 |
| 898 | 0.00058 | 18 | 0.04 | 0.38 |
| 899 | 0.00055 | 30 | 0.0068 | 0.15 |
| 900 | 0.0155 | 6719 | 0.76 | >60.0000 |
| 901 | 0.0014 | 557 | 0.03 | 0.28 |
| 902 | 0.42 | 3050 | 3.3 | >60.0000 |
| 903 | 0.0255 | 132 | 3.2 | 8 |
| 904 | 0.56 | 645 | 2.3 | 7.8 |
| 905 | 0.315 | 250 | 1.4 | 4.5 |
| 906 | 0.63 | 2043 | 5.2 | 23 |
| 907 | >0.800000 | 1846 | 5.5 | 7 |
| 908 | 0.02525 | >10000 | 1.3 | 17 |
| 909 | 0.01255 | 7034 | 1.2 | >60.0000 |
| 910 | 0.000335 | 14 | <0.00080 | 0.0008 |
| 911 | 0.0003 | 118 | 0.0008 | 0.01 |
| 912 | 0.00008 | 6 | <0.00080 | <0.0008 |
| 913 | 0.000115 | 10 | <0.00080 | 0.0013 |
| 914 | 0.00068 | 34 | <0.00080 | 0.02 |
| 915 | 0.000605 | 26 | 0.00077 | 0.025 |
| 916 | 0.000705 | 20 | 0.0013 | 0.11 |
| 917 | 0.00076 | 20 | 0.0014 | 0.06 |
| 918 | 0.00059 | 40 | 0.005 | 0.18 |
| 919 | 0.000455 | 8 | 0.031 | 0.42 |
| 920 | 0.000495 | 4 | 0.0008 | 0.3 |
| 921 | 0.00038 | 12 | 0.001 | 0.12 |
| 922 | 0.00059 | 30 | <0.00080 | 0.09 |
| 923 | 0.0037 | 737 | 0.11 | 1.2 |
| 924 | 0.0015 | 126 | 0.0013 | 0.11 |
| 925 | 0.0745 | 1646 | 2.5 | 10.5 |
| 926 | 0.05 | 442 | 3.7 | 8.5 |
| 927 | 0.00365 | 104 | 1.3 | 1.2 |
| 928 | 0.0575 | 2420 | 4.2 | 1 |
| 929 | 0.0985 | 939 | 3.5 | 9 |
| 930 | 0.0375 | 36 | | |
| 931 | 2.275 | 2003 | | |
| 932 | 0.01755 | | | |
| 933 | 0.0012 | | | |
| 934 | 0.335 | 4915 | 11 | 40 |
| 935 | 0.00445 | 118 | 0.28 | 1 |
| 936 | 0.1235 | 1264 | 33 | 40 |
| 937 | 5.95 | 466 | >60.00000 | >60.0000 |
| 938 | 0.0645 | 4207 | 4 | 11 |
| 939 | 0.00092 | 44 | 0.14 | 0.5 |
| 940 | 0.0605 | 401 | 1.95 | 6 |
| 941 | 0.084 | 3876 | 2 | 9 |
| 942 | 0.02 | 1389 | 2.8 | 10.2 |
| 943 | 0.0315 | 276 | 1.9 | 7.5 |
| 944 | 0.025 | 2656 | 0.9 | 2.1 |
| 945 | 0.19 | >10000 | 5 | 20 |
| 946 | 0.048 | 13000 | 1.6 | 7 |
| 947 | 0.0109 | 1596 | 0.98 | 4.8 |
| 948 | 0.00465 | 8827 | 0.22 | 0.7 |
| 949 | 0.056 | 1886 | 9.8 | 25 |
| 950 | 0.0355 | 9009 | 4.9 | 17 |
| 951 | 0.0845 | 3423 | 4 | 7.8 |
| 952 | 0.0013 | 130 | 0.06 | 0.75 |
| 953 | 0.000335 | 7 | 0.0008 | 0.014 |
| 954 | 0.00033 | 10 | 0.009 | 0.1 |
| 955 | 0.004 | 25 | 180 | 500 |
| 956 | 0.0004 | 1.75 | 47 | 90 |
| 957 | 0.034 | 480 | | |
| 958 | 1.4 | 2270 | | |
| 959 | 0.018 | 750 | | |
| 960 | 0.018 | ND | | |
| 961 | 0.0012 | ND | | |
| 962 | 0.065 | 1090 | 6 | 12 |
| 963 | 3.3 | 1625 | | |
| 964 | 0.042 | 70 | | |

Throughout this application, various publications are referenced. The disclosures of these publications in their entireties are hereby incorporated by reference into this application in order to more fully describe the state of the art as known to those skilled therein as of the date of the invention described and claimed herein.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A compound of the Formula (Ia): 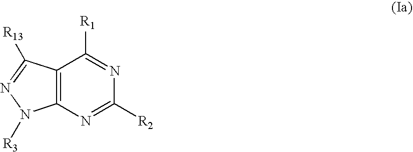
or a pharmaceutically acceptable salt or tautomer thereof,
wherein:
R₁ is 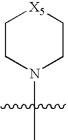
X₅ is —O—, —CH₂—O—, or —S(O)ₙ—, and any one or more of the ring hydrogen atoms of R₁ can independently be replaced with C₁-C₆alkyl, C₂-C₆alkenyl, C₂-C₆alkynyl, C₁-C₃alkoxy, C₁C₃acyl, C₁-C₃alkoxycarbonyl, amino(C₁-C₆alkyl), hydroxyl, fluorine, or —CN, where any two hydrogen atoms attached to the same carbon atom of R₁ can be replaced by an oxygen atom, the oxygen atom taken together with the carbon to which it is attached, forming a carbonyl (C═O), and wherein n is an integer from 0 to 2;
R₂ is
a) C₆-C₁₄aryl optionally substituted with from 1 to 3 substituents independently selected from:
(i) halogen,
(ii) C₁-C₆alkyl optionally substituted with from 1 to 3 substituents independently selected from halogen, heterocycle, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl,
(iii) C₁-C₆alkoxy optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl,
(iv) C₁-C₆alkoxycarbonyl,
(v) C₂-C₆alkenyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl,
(vi) C₂-C₆alkynyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(c₁-C₆alkyl), —C(O)(C₁-C₆alkyl), C₆-C₁₄aryl, C₁-C₉heteroaryl, and C₃-C₈cycloalkyl,
(vii) C₃-C₈cycloalkyl optionally substituted with from 1 to 3 substituents independently selected from C₁-C₆alkyl, halo, halo(C₁-C₆alkyl)-, hydroxyl, —O—C₁-C₆alkyl, —NH₂, amino(C₁-C₆alkyl)-, (C₁-C₆alkyl)amino-, di(C₁-C₆alkyl)amino-, —COOH, —C(O)O—(C₁-C₆alkyl), —OC(O)—(C₁-C₆alkyl), (C₁-C₆alkyl)carboxyamido-, —C(O)NH₂, carboxyamidoalkyl- and —NO₂,
(viii) C₆-C₁₄aryl optionally substituted with from 1 to 3 substituents independently selected from C₁-C₆alkyl, halo, halo(C₁-C₆alkyl)-, hydroxyl, C₁-C₆hydroxylalkyl, —NH₂, amino(C₁-C₆alkyl)-, (C₁-C₆alkyl)amino-, di(C₁-C₆alkyl)amino-, —COOH, —C(O)O—(C₁-C₆alkyl), —OC(O)—(C₁-C₆alkyl), (C₁-C₆alkyl)carboxyamido-, —C(O)NH₂, (C₁-C₆alkyl)N-alkylamido-, and —NO₂,
(ix) C₁-C₉heteroaryl optionally substituted with from 1 to 3 substituents independently selected from C₁-C₆alkyl, halo, halo(C₁-C₆alkyl)-, hydroxyl, C₁-C₆hydroxylalkyl, —NH₂, amino(C₁-C₆alkyl)-, (C₁-C₆alkyl)amino-, di(C₁-C₆alkyl)amino-, —COOH, —C(O)O—(C₁-C₆alkyl), —OC(O)—(C₁-C₆alkyl), (C₁-C₆alkyl)carboxyamido-, —C(O)NH₂, (C₁-C₆alkyl)N-alkylamido-, and —NO₂,
(x) C₁-C₆perfluoroalkyl-,
(xi) hydroxyl,
(xii) NR¹⁶R¹⁷,
(xiii) NO₂,
(xiv) CN,
(xv) CO₂H,
(xvi) CF₃,
(xvii) CF₃O,
(xviii) C₁-C₆alkylthio,
(xix) —SO₂NR¹⁶R¹⁷,
(xx) —O—C(O)NR¹⁶R¹⁷,
(xxi) —C(O)NR¹⁶R¹⁷,
(xxii) NR¹⁷C(O)R¹⁶,
(xxiii) N(C₁-C₆alkyl)C(O)R¹⁶,
(xxiv) —NHC(O)NR¹⁶R¹⁷,
(xxv) —NHC(O)NHNR¹⁶R¹⁷,
(xxvi) —NHC(O)OR¹⁸,
(xxvii) —NHC(O)NHOR¹⁶,
(xxviii) —NH(SO₂)NH—(C₁-C₆alkyl),
(xxix) —NH(SO₂)—(C₁-C₆alkyl),
(xxx) —NH(SO₂)NH—C₆-C₁₄aryl,
(xxxi) —NHC(S)—NH—C₁-C₆alkyl,
(xxxii) —N═C(S—C₁-C₆alkyl)(NH—C₁-C₆alkyl),
(xxxiii) —S(O)ₚ—C₆-C₁₄aryl,
(xxxiv) —S(O)ₚ—C₁-C₉heteroaryl,
(xxxv) —N(H)—C(═N—(CN))—(NR₁₆R₁₇), and
(xxxvi) —N(H)—C(═N—(CN))—(O—R¹⁶);
b) C₁-C₉heteroaryl optionally substituted with from 1 to 3 substituents independently selected from:
(i) halogen,
(ii) C₁-C₆alkyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl,
(iii) C₁-C₆alkoxy optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl,
(iv) C₁-C₆alkoxycarbonyl,
(v) C₂-C₆alkenyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl,
(vi) C₂-C₆alkynyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl,
(vii) C₃-C₈cycloalkyl optionally substituted with from 1 to 3 substituents independently selected from C₁-C₆alkyl, halo, halo(C₁-C₆alkyl)-, hydroxyl, —O—C₁-C₆alkyl, —NH₂, amino(C₁-C₆alkyl)-, (C₁-C₆alkyl)amino-, di(C₁-C₆alkyl)amino-, —COOH, —C(O)O—(C₁-C₆alkyl), —OC(O)—(C₁-C₆alkyl), (C₁-C₆alkyl)carboxyamido-, —C(O)NH₂, carboxyamidoalkyl- and —NO₂,
(viii) C₆-C₁₄aryl optionally substituted with from 1 to 3 substituents independently selected from C₁-C₆alkyl, halo, halo(C₁-C₆alkyl)-, hydroxyl, C₁-C₆hydroxylalkyl, —NH₂, amino(C₁-C₆alkyl)-, (C₁-C₆alkyl)amino-, di(C₁-C₆alkyl)amino-, —COOH, —C(O)O—(C₁-C₆alkyl), —OC(O)—(C₁-C₆alkyl), (C₁-C₆alkyl)carboxyamido-, —C(O)NH₂, (C₁-C₆alkyl)N-alkylamido-, and —NO₂,
(ix) C₁-C₉heteroaryl optionally substituted with from 1 to 3 substituents independently selected from C₁-C₆alkyl, halo, halo(C₁-C₆alkyl)-, hydroxyl, C₁-C₆hydroxylalkyl, —NH₂, amino(C₁-C₆alkyl)-, (C₁-C₆alkyl)amino-, di(C₁-C₆alkyl)amino-, —COOH, —C(O)O—(C₁-C₆alkyl), —OC(O)—(C₁-C₆alkyl), (C₁-C₆alkyl)carboxyamido-, —C(O)NH₂, (C₁-C₆alkyl)N-alkylamido-, and —NO₂,
(x) C₁-C₆perfluoroalkyl-,
(xi) hydroxyl,
(xii) NR¹⁶R¹⁷
(xiii) NO₂,
(xiv) CN,
(xv) CO₂H,
(xvi) CF₃,
(xvii) CF₃O,
(xviii) C₁-C₆alkylthio,
(xix) —SO₂NR¹⁶R¹⁷,
(xx) —C(O)NR¹⁶R¹⁷,
(xxi) NR¹⁷C(O)R¹⁶,
(xxii) —NHC(O)NR¹⁶R¹⁷,
(xxiii) —NHC(O)NHNR¹⁶R¹⁷,
(xxiv) —NHC(O)OR¹⁸,
(xxv) —NHC(O)NHOR¹⁶,
(xxvi) —NH(SO₂)NH—C₁-C₆alkyl,
(xxvii) —NH(SO₂)NH—C₆-C₁₄aryl,
(xxviii) —NHC(S)—NH—C₁-C₆alkyl,
(xxix) —N═C(S—C₁-C₆alkyl)(NH—C₁-C₆alkyl),
(xxx) —S(O)ₚ—C₆-C₁₄aryl,
(xxxi) —S(O)ₚ—C₁-C₉heteroaryl,
(xxxii) —N(H)—C(═N—(CN))—(NR¹⁶R¹⁷),
(xxxiii) —N(H)—C(═N—(CN))—(O—R¹⁶), and
(xxxiv) —N(H)—C(═N—(CN))—(O—C₆-C₁₄aryl);
c) —HC═CH—C₆-C₁₄aryl;
d) heterocycle attached by a ring carbon atom of the heterocycle;
e) or —HC═CH—C₁-C₉heteroaryl;
R¹⁶ and R¹⁷ are each independently
a) H;
b) C₁-C₆alkoxy;
c) C₁-C₆perfluoroalkyl;
d) C₆-C₁₄aryl optionally substituted with from 1 to 3 substituents independently selected from:
(i) C₁-C₆alkyl wherein the C₁-C₆alkyl is optionally substituted with one or more substituents independently selected from:
A) heterocycle optionally substituted with C₁-C₆alkyl,
B) NH₂—,
C) (C₁-C₆alkyl)amino-, and
D) di(C₁-C₆alkyl)amino-,
(ii) C₁-C₆alkoxy,
(iii) halo,
(iv) halo(C₁-C₆alkyl)-,
(v) hydroxyl,
(vi) C₁-C₆hydroxylalkyl,
(vii) heterocycle optionally substituted by C₁-C₆alkyl,
(viii) NH₂—,
(ix) amino(C₁-C₆alkyl)-,
(x) (C₁-C₆alkyl)amino-,
(xi) di(C₁-C₆alkyl)amino-,
(xii) C₁-C₆alkoxy-C₁-C₆alkylene-NH—C₁-C₆alkylene-,
(xiii) C₁-C₆hydroxylalkyl-NH—C₁-C₆alkylene-,
(xiv) amino(C₁-C₆alkyl)-NH—C₁-C₆alkylene-,
(xv) di(C₁-C₆alkyl)amino-C₁-C₆alkylene-NH—C₁-C₆alkylene-,
(xvi) C₁-C₆hydroxylalkyl-NH—,
(xvii) amino(C₁-C₆alkyl)-NH—,
(xviii) —COOH,
(xix) —C(O)O—(C₁-C₆alkyl),
(xx) —OC(O)—(C₁-C₆alkyl),
(xxi) (C₁-C₆alkyl)carboxyamido-,
(xxii) —C(O)NH₂,
(xxiii) (C₁-C₆alkyl)N-alkylamido-,
(xxiv) C₁-C₆alkoxy, and
(xxv) —NO₂;
e) C₁-C₉heteroaryl optionally substituted with from 1 to 3 substituents independently selected from:
(i) C₁-C₆alkyl,
(ii) C₁-C₆alkoxy,
(iii) halo,
(iv) halo(C₁-C₆alkyl)-,
(v) hydroxyl,
(vi) C₁-C₆hydroxylalkyl,
(vii) NH₂—,
(viii) amino(C₁-C₆alkyl)-,
(ix) (C₁-C₆alkyl)amino-,
(x) di(C₁-C₆alkyl)amino-,
(xi) —COOH,
(xii) —C(O)O—(C₁-C₆alkyl),
(xiii) —OC(O)—(C₁-C₆alkyl),
(xiv) (C₁-C₆alkyl)carboxyamido-,
(xv) —C(O)NH₂,
(xvi) (C₁-C₆alkyl)N-alkylamido-, and
(xvii) —NO₂;
f) C₃-C₈cycloalkyl optionally substituted with from 1 to 3 substituents independently selected from:
(i) C₁-C₆alkyl,
(ii) C₁-C₆alkoxy,
(iii) halo,
(iv) halo(C₁-C₆alkyl)-,
(v) hydroxyl,
(vi) —O—C₁-C₆alkyl,
(vii) —NH₂,
(viii) -amino(C₁-C₆alkyl),
(ix) (C₁-C₆alkyl)amino-,
(x) di(C₁-C₆alkyl)amino-,
(xi) —COOH,
(xii) —C(O)O—(C₁-C₆alkyl),
(xiii) —OC(O)—(C₁-C₆alkyl),
(xiv) (C₁-C₆alkyl)carboxyamido-,
(xv) —C(O)NH₂,
(xvi) carboxyamidoalkyl-, and
(xvii) —NO₂;
g) C₁-C₆alkyl optionally substituted with from 1 to 3 substituents independently selected from:
(i) halogen,
(ii) —NH₂,
(iii) —NH(C₁-C₆alkyl),
(iv) —N(C₁-C₆alkyl)(C₁-C₆alkyl),
(v) —CO₂H,
(vi) hydroxyl,
(vii) heterocycle,
(viii) C₁-C₆alkoxy,
(ix) C₆-C₁₄aryl wherein the C₆-C₁₄aryl is optionally substituted with di(C₁-C₆alkyl)amino-,
(x) C₁-C₉heteroaryl,
(xi) and C₃-C₈cycloalkyl;
h) C₂-C₆alkenyl optionally substituted with from 1 to 3 substituents independently selected from:
(i) halogen,
(ii) —NH₂,
(iii) —NH(C₁-C₆alkyl),
(iv) —N(C₁-C₆alkyl)(C₁-C₆alkyl),
(v) hydroxyl,
(vi) C₁-C₆alkoxy,
(vii) C₁-C₆alkyl,
(viii) C₆-C₁₄aryl,
(ix) C₁-C₉heteroaryl,
(x) and C₃-C₈cycloalkyl;
i) C₃-C₆alkynyl optionally substituted with from 1 to 3 substituents independently selected from:
(i) halogen,
(ii) —NH₂,
(iii) —NH(C₁-C₆alkyl),
(iv) —N(C₁-C₆alkyl)(C₁-C₆alkyl),
(v) hydroxyl,
(vi) C₁-C₆alkoxy,
(vii) C₁-C₆alkyl,
(viii) C₆-C₁₄aryl,
(ix) C₁-C₉heteroaryl,
(x) and C₃-C₈cycloalkyl;
j) heterocycle optionally substituted with from 1 to 3 substituents independently selected from:
(i) C₁-C₆alkyl,
(ii) C₆-C₁₄aryl,
(iii) and C₁-C₉heteroaryl;
or R¹⁶ and R¹⁷ when taken together with the nitrogen to which they are attached can form a 3- to 7-membered nitrogen containing heterocycle wherein up to two of the carbon atoms of the heterocycle can be replaced with —N(H)—, —N(C₁-C₆alkyl), —O—, or —S(O)ₚ—;
R¹⁸ is
a) C₁-C₆alkyl optionally substituted with from 1 to 3 substituents independently selected from:
(i) halogen,
(ii) —NH₂,
(iii) —NH(C₁-C₆alkyl),
(iv) —N(C₁-C₆alkyl)(C₁-C₆alkyl),
(v) —N(C₁-C₃alkyl)C(O)(C₁-C₆alkyl),
(vi) —NHC(O)(C₁-C₆alkyl),
(vii) —NHC(O)H,
(viii) —C(O)NH₂,
(ix) —C(O)NH(C₁-C₆alkyl),
(x) —C(O)N(C₁-C₆alkyl)(C₁-C₆alkyl),
(xi) —CN,
(xii) hydroxyl,
(xiii) C₁-C₆alkoxy,
(xiv) C₁-C₆alkyl,
(xv) —C(O)OH,
(xvi) —C(O)O(C₁-C₆alkyl),
(xvii) —C(O)(C₁-C₆alkyl),
(xviii) C₆-C₁₄aryl,
(xix) heterocycle optionally substituted by C₁-C₆alkyl,
(xx) C₁-C₉heteroaryl,
(xxi) and C₃-C₈cycloalkyl;
b) monocyclic C₁-C₆heterocycle optionally substituted with from 1 to 3 substituents independently selected from:
(i) C₁-C₈acyl,
(ii) C₁-C₆alkyl,
(iii) heteroaryl(C₁-C₆alkyl),
(iv) heterocyclyl(C₁-C₆alkyl),
(v) (C₆-C₁₄aryl)alkyl,
(vi) and (C₁-C₆alkoxy)carbonyl;
c) or C₆-C₁₄aryl optionally substituted with from 1 to 3 substituents independently selected from:
(i) C₁-C₆alkyl,
(ii) halo,
(iii) halo(C₁-C₆alkyl)-,
(iv) hydroxyl,
(v) C₁-C₆hydroxylalkyl,
(vi) —NH₂,
(vii) -amino(C₁-C₆alkyl),
(viii) (C₁-C₆alkyl)amino-,
(ix) di(C₁-C₆alkyl)amino-,
(x) —COOH,
(xi) —C(O)O—(C₁-C₆alkyl),
(xii) —OC(O)—(C₁-C₆alkyl),
(xiii) (C₁-C₆alkyl)carboxyamido-,
(xiv) —C(O)NH₂,
(xv) (C₁-C₆alkyl)N-alkylamido-, and
(xvi) —NO₂;
each p is independently 1 or 2;
R₃ is
a) hydrogen;
b) C₁-C₁₀alkyl optionally substituted with from 1 to 3 substituents independently selected from:
(i) C₁-C₆alkoxy,
(ii) NH₂,
(iii) (C₁-C₆alkyl)amino,
(iv) di(C₁-C₆alkyl)amino,
(v) heterocycle,
(vi) the group 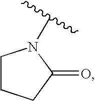
(vii) C(O)NH₂,
(viii) CO₂H,
(ix) and (C₁-C₆alkoxy)carbonyl;
c) C₂-C₁₀alkenyl;
d) C₂-C₁₀alkynyl;
e) C₁-C₈acyl;
f) C₆-C₁₄aryl;
g) C₁-C₉heteroaryl;
h) C₁-C₆hydroxylalkyl;
i) C₁-C₆alkylcarboxy;
j) C₁-C₆perfluoroalkyl;
k) —S(O)_{q}—(C₁-C₆alkyl);
l) —S(O)_{q}-aryl;
m) C₃-C₈carbocycle optionally substituted with from 1 to 3 substituents independently selected from:
(i) C₁-C₆alkoxy,
(ii) hydroxyl,
(iii) heterocycle,
(iv) (C₁-C₆alkoxy)-(C₆-C₁₄aryl)-NH—,
(v) NH₂,
(vi) (C₁-C₆alkyl)amino,
(vii) di(C₁-C₆alkyl)amino,
(viii) CO₂H,
(ix) and (C₁-C₆alkoxy)carbonyl;
or two hydrogen atoms on the same carbon atom of the C₃-C₈carbocycle can be replaced by an oxygen atom, the oxygen atom taken together with the carbon to which it is attached, forming a carbonyl (C═O) group, or two hydrogen atoms on the same carbon atom of the C₃-C₈carbocycle can be replaced by an alkylenedioxy group so that the alkylenedioxy group, when taken together with the carbon atom to which it is attached, form a 5- to 7-membered heterocycle containing two oxygen atoms;
n) 6- to 10-membered bicyclic carbocycle;
o) monocyclic C₁-C₆heterocycle optionally substituted with from 1 to 3 substituents independently selected from:
(i) C₁-C₈acyl, wherein the C₁-C₈acyl is optionally substituted with from 1 to 3 substituents independently selected from:
A) hydroxyl,
B) CN,
C) C₁-C₆alkoxy,
D) C₁-C₆alkyl,
E) C₁-C₈acyl,
F) NH₂,
G) (C₁-C₆alkyl)amino,
H) di(C₁-C₆alkyl)amino,
I) CO₂H,
J) (C₁-C₆alkoxy)carbonyl,
K) C₁-C₆perfluoroalkyl,
L) and halogen,
(ii) C₁-C₆alkyl optionally substituted with from 1 to 3 substituents independently selected from:
A) C₃-C₈cycloalkyl,
B) C₁-C₆alkoxy,
C) C₁-C₈acyl,
D) CN,
E) (C₁-C₆alkoxy)carbonyl,
F) CO₂H,
G) hydroxyl,
H) C₁-C₉heterocycle, and
I) H₂NC(O)—,
(iii) C₁-C₆perfluoroalkyl,
(iv) C₂-C₆alkenyl,
(v) heteroaryl(C₁-C₆alkyl) wherein the ring portion of the heteroaryl(C₁-C₆alkyl) group is optionally substituted with from 1 to 3 substituents independently selected from:
A) C₁-C₆alkylC(O)NH—,
B) C₁-C₆alkoxy,
C) halogen,
D) NH₂,
E) and C₁-C₆alkyl,
(vi) (C₆-C₁₄aryl)alkyl, wherein the ring portion of the (C₆-C₁₄aryl)alkyl group is optionally substituted by 1 to 3 substituents independently selected from:
A) halogen,
B) C₁-C₆alkyl,
C) NH₂,
D) (C₁-C₆alkyl)amino,
E) di(C₁-C₆alkyl)amino,
F) hydroxyl,
G) C₁-C₆alkoxy,
H) C₁-C₈acyl,
I) and C₁-C₉heteroaryl,
(vii) HC(O)—,
(viii) C₁-C₆perfluoroalkyl,
(ix) —S(O)_{q}—(C₁-C₆alkyl),
(x) —S(O)_{q}-aryl,
(xi) R¹⁹R²⁰NC(O),
(xii) (C₁-C₉heteroaryl)-NH—C(S)—,
(xiii) (C₁-C₆alkyl)-NH—C(S)—,
(xiv) (C₁-C₆alkyl)-S—C(O)—,
(xv) (C₆-C₁₄aryloxy)carbonyl,
(xvi) (C₂-C₆alkenyloxy)carbonyl,
(xvii) (C₂-C₆alkynyloxy)carbonyl,
(xviii) and (C₁-C₆alkoxy)carbonyl optionally substituted with from 1 to 3 substituents independently selected from:
A) C₁-C₆alkoxy,
B) halogen,
C) C₆-C₁₄aryl,
D) NH₂,
E) (C₁-C₆alkyl)amino-,
F) di(C₁-C₆alkyl)amino-,
G) and C₁-C₆alkyl;
p) or bicyclic C₁-C₉heterocycle;
R¹⁹ and R²⁰ are each independently:
a) H;
b) C₁-C₆alkyl optionally substituted with a substituent selected from:
(i) C₁-C₆alkylC(O)NH—,
(ii) NH₂,
(iii) (C₁-C₆alkyl)amino, and
(iv) di(C₁-C₆alkyl)amino;
c) C₃-C₈cycloalkyl;
d) C₆-C₁₄aryl optionally substituted with a substituent selected from:
(i) halogen,
(ii) and monocyclic C₁-C₆heterocycle wherein the monocyclic C₁-C₆heterocycle is optionally substituted with (C₁-C₆alkoxy)carbonyl;
e) C₁-C₉heteroaryl;
f) heteroaryl(C₁-C₆alkyl);
g) heterocyclyl(C₁-C₆alkyl);
h) (C₆-C₁₄aryl)alkyl, wherein the chain portion of the (C₆-C₁₄aryl)alkyl group is optionally substituted by a hydroxyl;
i) or monocyclic C₁-C₆heterocycle optionally substituted with a (C₁-C₆alkoxy)carbonyl;
or R¹⁹ and R²⁰ when taken together with the nitrogen to which they are attached optionally form a 3- to 7-membered nitrogen-containing heterocycle wherein up to two of the carbon atoms of the heterocycle are optionally replaced with —N(H)—, —N(C₁-C₆alkyl)-, —N(C₆-C₁₄aryl)-, or —O—, and wherein the nitrogen-containing heterocycle is optionally substituted by a C₁-C₆alkyl; C₆-C₁₄aryl, (C₁-C₆alkoxy)C(O)NH—, or C₁-C₉heterocycle;
R₁₃ is hydrogen, halogen, C₁-C₆alkyl, C₂-C₆alkenyl, C₂-C₆alkynyl, C₆-C₁₄aryl, or C₁-C₉heteroaryl; and where each C₁-C₆alkyl, C₂-C₆alkenyl, C₂-C₆alkynyl, C₆-C₁₄aryl, or C₁-C₉heteroaryl is optionally substituted by a C₁-C₆hydroxylalkyl, NH₂, (C₁-C₆alkyl)amino, or di(C₁-C₆alkyl)amino;
q is 1 or 2;
provided that the compound is not 4-(4-morpholinyl)-1-phenyl-6-[3-(trifluoromethyl)phenyl]-1H-pyrazolo[3,4-d]pyrimidine.

2. The compound of claim 1, wherein X₅ is —O—.

3. The compound of claim 1, wherein R₂ is C₆-C₁₄aryl optionally independently substituted with from 1 to 3 substituents as specified in claim 1.

4. The compound of claim 1, wherein R₂ is C₆-C₁₄aryl substituted by —NHC(O)NHNR¹⁶R¹⁷.

5. The compound of claim 1, wherein R₂ is C₆-C h₄aryl substituted by —NHC(O)OR¹⁸.

6. The compound of claim 1, wherein R₃ is hydrogen.

7. The compound of claim 1, wherein R₃ is C₆-C₁₄aryl.

8. The compound of claim 1, wherein R₃ is monocyclic C₁-C₆heterocycle optionally independently substituted with from 1 to 3 substituents as specified in claim 1.

9. The compound of claim 8, wherein the monocyclic C₁-C₆heterocycle is a piperidine.

10. The compound of claim 9, wherein the C4 of the piperidine ring is directly bonded to N-1 of the 1H-pyrazolo[3,4-d]pyrimidine ring.

11. The compound of claim 9 wherein the piperidine nitrogen is further substituted with a substituent selected from:
(a) C₁-C₈acyl, wherein the C₁-C₈acyl is optionally substituted with from 1 to 3 substituents independently selected from:
(i) hydroxyl,
(ii) CN,
(iii) C₁-C₆alkoxy,
(iv) C₁-C₆alkyl,
(v) C₁-C₈acyl,
(vi) NH₂,
(vii) (C₁-C₆alkyl)amino,
(viii) di(C₁-C₆alkyl)amino,
(ix) CO₂H,
(x) (C₁-C₆alkoxy)carbonyl,
(xi) C₁-C₆perfluoroalkyl,
(xii) and halogen;
(b) C₁-C₆alkyl optionally substituted with from 1 to 3 substituents independently selected from:
(i) C₃-C₈cycloalkyl,
(ii) C₁-C₆alkoxy,
(iii) C₁-C₈acyl,
(iv) CN,
(v) (C₁-C₆alkoxy)carbonyl,
(vi) CO₂H,
(vii) hydroxyl,
(viii) C₁-C₉heterocycle, and
(ix) H₂NC(O)—;
(c) C₁-C₆perfluoroalkyl;
(d) C₂-C₆alkenyl;
(e) heteroaryl(C₁-C₆alkyl) wherein the ring portion of the heteroaryl(C₁-C₆alkyl) group is optionally substituted with from 1 to 3 substituents independently selected from:
(i) C₁-C₆alkylC(O)NH—,
(ii) C₁-C₆alkoxy,
(iii) halogen,
(iv) NH₂,
(v) and C₁-C₆alkyl;
(f) (C₆-C₁₄aryl)alkyl, wherein the ring portion of the (C₆-C₁₄aryl)alkyl group is optionally substituted by 1 to 3 substituents independently selected from:
(i) halogen,
(ii) C₁-C₆alkyl,
(iii) NH₂,
(iv) (C₁-C₆alkyl)amino,
(v) di(C₁-C₆alkyl)amino,
(vi) hydroxyl,
(vii) C₁-C₆alkoxy,
(viii) C₁-C₈acyl,
(ix) and C₁-C₉heteroaryl;
(g) HC(O)—;
(h) C₁-C₆perfluoroalkyl;
(i) —S(O)_{q}—(C₁-C₆alkyl);
(j) —S(O)_{q}-aryl;
(k) R¹⁹R²⁰NC(O);
(l) (C₁-C₉heteroaryl)-NH—C(S)—;
(m) (C₁-C₆alkyl)-NH—C(S)—;
(n) (C₁-C₆alkyl)-S—C(O)—;
(o) (C₆-C₁₄aryloxy)carbonyl;
(p) (C₂-C₆alkenyloxy)carbonyl;
(q) (C₂-C₆alkynyloxy)carbonyl;
(r) and (C₁-C₆alkoxy)carbonyl optionally substituted with from 1 to 3 substituents independently selected from:
(i) C₁-C₆alkoxy,
(ii) halogen,
(iii) C₆-C₁₄aryl,
(iv) NH₂,
(v) (C₁-C₆alkyl)amino-, and
(vi) di(C₁-C₆alkyl)amino-; and
(s) C₁-C₆alkyl.

12. The compound of claim 1, wherein R₃ is monocyclic C₁-C₆heterocycle optionally substituted with from 1 to 3 substituents as specified in claim 1 and X₅ is —O—.

13. The compound of claim 1, wherein R₂ is C₆-C₁₄aryl optionally independently substituted with from 1 to 3 substituents as specified in claim 1 and R₃ is monocyclic C₁-C₆heterocycle optionally independently substituted with from 1 to 3 substituents as specified in claim 1.

14. The compound of claim 1, wherein R₂ is C₆-C₁₄aryl substituted by —NHC(O)NHNR¹⁶R¹⁷ and R₃ is monocyclic C₁-C₆heterocycle optionally substituted with from 1 to 3 substituents as specified in claim 1.

15. The compound of claim 1, wherein R₂ is C₆-C₁₄aryl substituted by —NHC(O)OR¹⁸ and R₃ is monocyclic C₁-C₆heterocycle optionally substituted with from 1 to 3 substituents as specified in claim 1.

16. The compound of claim 1, wherein R₂ is C₁-C₉heteroaryl optionally independently substituted with from 1 to 3 substituents as specified in claim 1 and R₃ is monocyclic C₁-C₆heterocycle optionally substituted with from 1 to 3 substituents as specified in claim 1.

17. A compound of Formula IIIb: 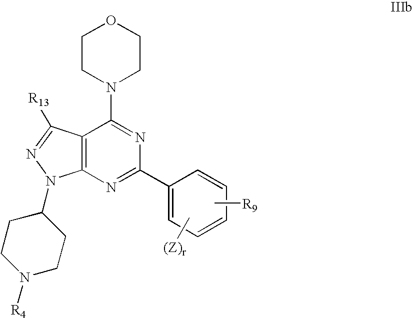
or a pharmaceutically acceptable salt or tautomer thereof,
wherein
R₄ is selected from:
a) hydrogen;
b) C₁-C₈acyl, wherein the C₁-C₈acyl is optionally substituted with from 1 to 3 substituents independently selected from:
(i) hydroxyl,
(ii) CN,
(iii) C₁-C₆alkoxy,
(iv) C₁-C₆alkyl,
(v) C₁-C₈acyl,
(vi) NH₂,
(vii) (C₁-C₆alkyl)amino,
(viii) di(C₁-C₆alkyl)amino,
(ix) CO₂H,
(x) (C₁-C₆alkoxy)carbonyl,
(xi) C₁-C₆perfluoroalkyl,
(xii) and halogen;
c) C₁-C₆alkyl optionally substituted with from 1 to 3 substituents independently selected from:
(i) C₃-C₈cycloalkyl,
(ii) C₁-C₆alkoxy,
(iii) C₁-C₈acyl,
(iv) CN,
(v) (C₁-C₆alkoxy)carbonyl,
(vi) CO₂H,
(vii) hydroxyl,
(viii) C₁-C₉heterocycle, and
(ix) H₂NC(O)—;
d) C₁-C₆perfluoroalkyl;
e) C₂-C₆alkenyl;
f) heteroaryl(C₁-C₆alkyl) wherein the ring portion of the heteroaryl(C₁-C₆alkyl) group is optionally substituted with from 1 to 3 substituents independently selected from:
(i) C₁-C₆alkylC(O)NH—,
(ii) C₁-C₆alkoxy,
(iii) halogen,
(iv) NH₂,
(v) and C₁-C₆alkyl;
g) (C₆-C₁₄aryl)alkyl, wherein the ring portion of the (C₆-C₁₄aryl)alkyl group is optionally substituted by 1 to 3 substituents independently selected from:
(i) halogen,
(ii) C₁-C₆alkyl,
(iii) NH₂,
(iv) (C₁-C₆alkyl)amino,
(v) di(C₁-C₆alkyl)amino,
(vi) hydroxyl,
(vii) C₁-C₆alkoxy,
(viii) C₁-C₈acyl,
(ix) and C₁-C₉heteroaryl;
h) HC(O)—;
i) C₁-C₆perfluoroalkyl;
j) —S(O)_{q}—(C₁-C₆alkyl);
k) —S(O)_{q}-aryl;
l) R¹⁹R²⁰NC(O);
m) (C₁-C₉heteroaryl)-NH—C(S)—;
n) (C₁-C₆alkyl)-NH—C(S)—;
o) (C₁-C₆alkyl)-S—C(O)—;
p) (C₆-C₁₄aryloxy)carbonyl;
q) (C₂-C₆alkenyloxy)carbonyl;
r) (C2-C6alkynyloxy)carbonyl;
s) and (C₁-C₆alkoxy)carbonyl optionally substituted with from 1 to 3 substituents independently selected from:
(i) C₁-C₆alkoxy,
(ii) halogen,
(iii) C₆-C₁₄aryl,
(iv) NH₂,
(v) (C₁-C₆alkyl)amino-,
(vi) di(C₁-C₆alkyl)amino-,
(vii) and C₁-C₆alkyl;
R₉ is —OH, —NHC(O)NR₁₀R₁₁, —NHC(O)OR₁₂, —NH(SO₂)NHalkyl, —NH(SO₂)NHaryl, —NHC(S)—NHalkyl, —N═C(Salkyl)(NHalkyl), or —N(H)—C(═N—(CN))—(Oaryl);
R₁₀ and R₁₁ are each independently —H, —OH, C₁-C₆alkoxy, C₆-C₁₄aryl, C₁-C₉heteroaryl, C₃-C₈carbocycle, or C₁-C₆alkyl; or R₁₀ and R₁₁ when taken together with the nitrogen to which they are attached to form a 3- to 7-membered nitrogen containing heterocycle wherein up to two of the carbon atoms of the heterocycle may be substituted with —N(R₁₅)—, —O—, or —S(O)ₙ;
R₁₂ is C₁-C₆alkyl, C₁-C₆hydroxylalkyl, or C₆-C₁₄aryl;
R₁₃ is hydrogen, halogen, C₁-C₆alkyl, C₂-C₆alkenyl, C₂-C₆alkynyl, C₆-C₁₄aryl, or C₁-C₉heteroaryl, where each C₁-C₆alkyl, C₂-C₆alkenyl, C₂-C₆alkynyl, C₆-C₁₄aryl, or C₁-C₉heteroaryl is optionally substituted by a C₁-C₆hydroxylalkyl, NH₂, (C₁-C₆alkyl)amino, or di(C₁-C₆alkyl)amino;
R₁₅ is hydrogen, C₁-C₆alkyl, C₃-C₈ carbocycle, C₆-C₁₄aryl, C₁-C₉heteroaryl, (C₁-C₆alkyl)amino, or (C₆-C₁₄aryl)amino;
n is 0, 1, or 2;
q is 1 or 2;
R¹⁹ and R²⁰ are each independently:
a) H;
b) C₁-C₆alkyl optionally substituted with a substituent selected from:
(i) C₁-C₆alkylC(O)NH—,
(ii) NH₂,
(iii) (C₁-C₆alkyl)amino, and
(iv) di(C₁-C₆alkyl)amino;
c) C₃-C₈cycloalkyl;
d) C₆-C₁₄aryl optionally substituted with a substituent selected from:
(i) halogen,
(ii) and monocyclic C₁-C₆heterocycle wherein the monocyclic C₁-C₆heterocycle is optionally substituted with (C₁-C₆alkoxy)carbonyl;
e) C₁-C₉heteroaryl;
f) heteroaryl(C₁-C₆alkyl);
g) heterocyclyl(C₁-C₆alkyl);
h) (C₆-C₁₄aryl)alkyl, wherein the chain portion of the (C₆-C₁₄aryl)alkyl group is optionally substituted by a hydroxyl;
i) or monocyclic C₁-C₆heterocycle optionally substituted with a (C₁-C₆alkoxy)carbonyl;
or R¹⁹ and R²⁰ when taken together with the nitrogen to which they are attached optionally form a 3- to 7-membered nitrogen-containing heterocycle wherein up to two of the carbon atoms of the heterocycle are optionally replaced with —N(H)—, —N(C₁-C₆alkyl)-, —N(C₆-C₁₄aryl)-, or —O—, and wherein the nitrogen-containing heterocycle is optionally substituted by a C₁-C₆alkyl; C₆-C₁₄aryl, (C₁-C₆alkoxy)C(O)NH—, or C₁-C₉heterocycle;
r is 0 or 1; and
Z is halogen, C₁-C₆alkyl, or C₁-C₆alkoxy.

18. The compound of claim 17, wherein R₄ is C₁-C₈acyl, wherein the C₁-C₈acyl is optionally independently substituted with from 1 to 3 substituents as specified in claim 17, heteroaryl(C₁-C₆alkyl) wherein the ring portion of the heteroaryl(C₁-C₆alkyl) group is optionally independently substituted with from 1 to 3 substituents as specified in claim 17, (C₆-C₁₄aryl)alkyl wherein the ring portion of the (C₆-C₁₄aryl)alkyl group is optionally independently substituted with from 1 to 3 substituents as specified in claim 17, or (C₁-C₆alkoxy)carbonyl wherein the (C₁-C₆alkoxy)carbonyl is optionally independently substituted with from 1 to 3 substituents as specified in claim 17.

19. The compound of claim 18, wherein R₄ is C₁-C₈acyl, wherein the C₁-C₈acyl is optionally independently substituted with from 1 to 3 substituents as specified in claim 17.

20. The compound of claim 18, wherein R₄ is heteroaryl(C₁-C₆alkyl) wherein the ring portion of the heteroaryl(C₁-C₆alkyl) group is optionally independently substituted with from 1 to 3 substituents as specified in claim 17, or (C₆-C₁₄aryl)alkyl wherein the ring portion of the (C₆-C₁₄aryl)alkyl group is optionally independently substituted with from 1 to 3 substituents as specified in claim 17.

21. The compound of claim 17, wherein R₉ is —NHC(O)NR₁₀R₁₁ or —NHC(O))R₁₂.

22. The compound of claim 21, wherein R₁₀ is hydrogen and R₁₁ is selected from the group consisting of C₆-C₁₄aryl, C₁-C₉heteroaryl, C₃-C₈carbocycle, and C₁-C₆alkyl.

23. The compound of claim 22, wherein R₁₁ is ethyl or 4-pyridyl.

24. The compound of claim 21, wherein R₁₂ is C₁-C₆hydroxylalkyl.

25. A compound selected from the group consisting of
1-(4-{4-morpholin-4-yl-1-[1-(pyridin-3-ylcarbonyl)piperidin-4-yl]-1H-pyrazolo[3,4-d]pyrimidin-6-yl}phenyl)-3-pyridin-3-ylurea;
1-(4-{4-morpholin-4-yl-1-[1-(pyridin-3-ylcarbonyl)piperidin-4-yl]-1H-pyrazolo[3,4-d]pyrimidin-6-yl}phenyl)-3-(3-thienyl)urea
1-(4-{4-morpholin-4-yl-1-[1-(pyridin-3-ylcarbonyl)piperidin-4-yl]-1H-pyrazolo[3,4-d]pyrimidin-6-yl}phenyl)-3-pyridin-4-ylurea
1-(4-{4-morpholin-4-yl-1-[1-(pyridin-3-ylcarbonyl)piperidin-4-yl]-1H-pyrazolo[3,4-d]pyrimidin-6-yl}phenyl)-3-(2-thienyl)urea;
1-{4-[1-(1-benzoylpiperidin-4-yl)-4-morpholin-4-yl-1H-pyrazolo[3,4-d]pyrimidin-6-yl]phenyl}-3-ethylurea;
1-{4-[1-(1-benzoylpiperidin-4-yl)-4-morpholin-4-yl-1H-pyrazolo[3,4-d]pyrimidin-6-yl]phenyl}-3-methylurea 1-(4-{4-morpholin-4-yl-1-[1-(pyridin-3-ylcarbonyl)piperidin-4-yl]-1H-pyrazolo[3,4-d]pyrimidin-6-yl}phenyl)-3-phenylurea
1-{4-[1-(1-benzoylpiperidin-4-yl)-4-morpholin-4-yl-1H-pyrazolo[3,4-d]pyrimidin-6-yl]phenyl}-3-(2-fluoroethyl)urea
1-methyl-3-(4-{1-[1-(2-methylbenzoyl)piperidin-4-yl]-4-morpholin-4-yl-1H-pyrazolo[3,4-d]pyrimidin-6-yl}phenyl)urea;
1-(4-{1-[1-(2-chlorobenzoyl)piperidin-4-yl]-4-morpholin-4-yl-1H-pyrazolo[3,4-d]pyrimidin-6-yl}phenyl)-3-methylurea;
1-{4-[1-(1-benzoylpiperidin-4-yl)-4-morpholin-4-yl-1H-pyrazolo[3,4-d]pyrimidin-6-yl]phenyl}-3-pyridin-3-ylurea
1-{4-[1-(1-benzoylpiperidin-4-yl)-4-morpholin-4-yl-1H-pyrazolo[3,4-d]pyrimidin-6-yl]phenyl}-3-phenylurea
1-{4-[1-(1-benzoylpiperidin-4-yl)-4-morpholin-4-yl-1H-pyrazolo[3,4-d]pyrimidin-6-yl]phenyl}-3-(2-hydroxyethyl)urea
1-methyl-3-(4-{4-morpholin-4-yl-1-[1-(pyridin-2-ylcarbonyl)piperidin-4-yl]-1H-pyrazolo[3,4-d]pyrimidin-6-yl}phenyl)urea
and 1-(4-{1-[1-(2-fluorobenzoyl)piperidin-4-yl]-4-morpholin-4-yl-1H-pyrazolo[3,4-d]pyrimidin-6-yl}phenyl)-3-methylurea.
1-(4-{4-morpholin-4-yl-1-[1-(pyridin-3-ylmethyl)piperidin-4-yl]-1H-pyrazolo[3,4-d]pyrimidin-6-yl}phenyl)-3-pyridin-4-ylurea
1-(4-{1-[1-(3-fluorobenzoyl)piperidin-4-yl]-4-morpholin-4-yl-1H-pyrazolo[3,4-d]pyrimidin-6-yl}phenyl)-3-methylurea
1-{4-[1-(1-benzoylpiperidin-4-yl)-4-morpholin-4-yl-1H-pyrazolo[3,4-d]pyrimidin-6-yl]phenyl}-3-pyridin-4-ylurea
1-(4-{4-morpholin-4-yl-1-[1-(pyridin-3-ylmethyl)piperidin-4-yl]-1H-pyrazolo[3,4-d]pyrimidin-6-yl}phenyl)-3-pyridin-3-ylurea
1-(2-fluoroethyl)-3-(4-{4-morpholin-4-yl-1-[1-(pyridin-3-ylcarbonyl)piperidin-4-yl]-1H-pyrazolo[3,4-d]pyrimidin-6-yl}phenyl)urea.
1-(4-{4-morpholin-4-yl-1-[1-(pyridin-3-ylmethyl)piperidin-4-yl]-1H-pyrazolo[3,4-d]pyrimidin-6-yl}phenyl)-3-(2-thienyl)urea;
1-ethyl-3-(4-{4-morpholin-4-yl-1-[1-(pyridin-3-ylcarbonyl)piperidin-4-yl]-1H-pyrazolo[3,4-d]pyrimidin-6-yl}phenyl)urea;
1-{4-[1-(1-benzylpiperidin-4-yl)-4-morpholin-4-yl-1H-pyrazolo[3,4-d]pyrimidin-6-yl]phenyl}-3-(2-hydroxyethyl)urea;
1-{4-[1-(1,4-dioxaspiro[4.5]dec-8-yl)-4-morpholin-4-yl-1H-pyrazolo[3,4-d]pyrimidin-6-yl]phenyl}-3-methylurea;
1-[4-(1-{1-[(2-chloropyridin-3-yl)methyl]piperidin-4-yl}-4-morpholin-4-yl-1H-pyrazolo[3,4-d]pyrimidin-6-yl)phenyl]-3-methylurea;
1-(2-hydroxyethyl)-3-(4-{4-morpholin-4-yl-1-[1-(pyridin-3-ylcarbonyl)piperidin-4-yl]-1H-pyrazolo[3,4-d]pyrimidin-6-yl}phenyl)urea;
1-cyclopropyl-3-(4-{4-morpholin-4-yl-1-[1-(pyridin-3-ylcarbonyl)piperidin-4-yl]-1H-pyrazolo[3,4-d]pyrimidin-6-yl}phenyl)urea;
1-{4-[1-(1-benzoylpiperidin-4-yl)-4-morpholin-4-yl-1H-pyrazolo[3,4-d]pyrimidin-6-yl]phenyl}urea;
1-(4-{1-[1-(3-chlorobenzoyl)piperidin-4-yl]-4-morpholin-4-yl-1H-pyrazolo[3,4-d]pyrimidin-6-yl}phenyl)-3-methylurea;
1-(4-{4-morpholin-4-yl-1-[1-(pyridin-3-ylcarbonyl)piperidin-4-yl]-1H-pyrazolo[3,4-d]pyrimidin-6-yl}phenyl)-3-pyridin-2-ylurea;
1-[4-(1-{1-[(6-bromopyridin-3-yl)methyl]piperidin-4-yl}-4-morpholin-4-yl-1H-pyrazolo[3,4-d]pyrimidin-6-yl)phenyl]-3-methylurea;
1-methyl-3-(4-{4-morpholin-4-yl-1-[1-(pyridin-3-ylmethyl)piperidin-4-yl]-1H-pyrazolo[3,4-d]pyrimidin-6-yl}phenyl)urea;
1-methyl-3-[4-(1-{1-[(2-methylpyridin-3-yl)carbonyl]piperidin-4-yl}-4-morpholin-4-yl-1H-pyrazolo[3,4-d]pyrimidin-6-yl)phenyl]urea;
1-methyl-3-[4-(1-{1-[(4-methylpyridin-3-yl)carbonyl]piperidin-4-yl}-4-morpholin-4-yl-1H-pyrazolo[3,4-d]pyrimidin-6-yl)phenyl]urea;
1-methyl-3-(4-{4-morpholin-4-yl-1-[1-(pyridin-2-ylmethyl)piperidin-4-yl]-1H-pyrazolo[3,4-d]pyrimidin-6-yl}phenyl)urea;
1-(4-{1-[1-(2-methoxybenzoyl)piperidin-4-yl]-4-morpholin-4-yl-1H-pyrazolo[3,4-d]pyrimidin-6-yl}phenyl)-3-methylurea;
1-(4-{1-[1-(3-acetylbenzoyl)piperidin-4-yl]-4-morpholin-4-yl-1H-pyrazolo[3,4-d]pyrimidin-6-yl}phenyl)-3-methylurea;
1-(2-fluoro-4-{4-morpholin-4-yl-1-[1-(pyridin-3-ylmethyl)piperidin-4-yl]-1H-pyrazolo[3,4-d]pyrimidin-6-yl}phenyl)-3-pyridin-3-ylurea;
2-[4-(6-{4-[(methylcarbamoyl)amino]phenyl}-4-morpholin-4-yl-1H-pyrazolo[3,4-d]pyrimidin-1-yl)piperidin-1-yl]acetamide;
methyl 4-(6-{4-[(methylcarbamoyl)amino]phenyl}-4-morpholin-4-yl-1H-pyrazolo[3,4-d]pyrimidin-1-yl)piperidine-1-carboxylate;
1-methyl-3-{4-[4-morpholin-4-yl-1-(4-oxocyclohexyl)-1H-pyrazolo[3,4-d]pyrimidin-6-yl]phenyl}urea
1-(4-{1-[1-(2-chlorobenzyl)piperidin-4-yl]-4-morpholin-4-yl-1H-pyrazolo[3,4-d]pyrimidin-6-yl}phenyl)-3-methylurea
1-methyl-3-(4-{1-[1-(3-methylbenzoyl)piperidin-4-yl]-4-morpholin-4-yl-1H-[3,4-d]pyrimidin-6-yl}phenyl)urea
1-[4-(1-{1-[(6-chloropyridin-3-yl)methyl]piperidin-4-yl}-4-morpholin-4-yl-1H-pyrazolo[3,4-d]pyrimidin-6-yl)phenyl]-3-methylurea
1-methyl-3-[4-(4-morpholin-4-yl-1-{1-[3-(trifluoromethyl)benzoyl]piperidin-4-yl}-1H-pyrazolo[3,4-d]pyrimidin-6-yl)phenyl]urea
1-(4-{4-morpholin-4-yl-1-[1-(pyridin-3-ylmethyl)piperidin-4-yl]-1H-pyrazolo[3,4-d]pyrimidin-6-yl}phenyl)-3-phenylurea
1-methyl-3-[4-(1-{1-[(6-methylpyridin-3-yl)carbonyl]piperidin-4-yl}-4-morpholin-4-yl-1H-pyrazolo[3,4-d]pyrimidin-6-yl)phenyl]urea
3-(4-morpholin-4-yl-1H-pyrazolo[3,4-d]pyrimidin-6-yl)phenol
1-methyl-3-(4-{4-morpholin-4-yl-1-[1-(2-oxo-2-phenylethyl)piperidin-4-yl]-1H-pyrazolo[3,4-d]pyrimidin-6-yl}phenyl)urea
N-{4-[1-(1-benzylpiperidin-4-yl)-4-morpholin-4-yl-1H-pyrazolo[3,4-d]pyrimidin-6-yl]phenyl}-N′-methylurea;
1-(4-{1-[1-(3-methoxybenzoyl)piperidin-4-yl]-4-morpholin-4-yl-1H-pyrazolo[3,4-d]pyrimidin-6-yl}phenyl)-3-methylurea;
1-ethyl-3-(4-{4-morpholin-4-yl-1-[1-(pyridin-3-ylmethyl)piperidin-4-yl]-1H-pyrazolo[3,4-d]pyrimidin-6-yl}phenyl)urea;
1-[4-(1-{1-[(5-bromopyridin-3-yl)methyl]piperidin-4-yl}-4-morpholin-4-yl-1H-pyrazolo[3,4-d]pyrimidin-6-yl)phenyl]-3-methylurea;
1-(4-{4-morpholin-4-yl-1-[1-(pyridin-3-ylcarbonyl)piperidin-4-yl]-1H-pyrazolo[3,4-d]pyrimidin-6-yl}phenyl)urea;
1-{4-[1-(4-hydroxycyclohexyl)-4-morpholin-4-yl-1H-pyrazolo[3,4-d]pyrimidin-6-yl]phenyl}-3-methylurea;
1-{4-[1-(1-isonicotinoylpiperidin-4-yl)-4-morpholin-4-yl-1H-pyrazolo[3,4-d]pyrimidin-6-yl]phenyl}-3-methylurea;
1-[4-(1-{1-[(6-fluoropyridin-3-yl)carbonyl]piperidin-4-yl}-4-morpholin-4-yl-1H-pyrazolo[3,4-d]pyrimidin-6-yl)phenyl]-3-methylurea;
1-(2-fluoro-4-{4-morpholin-4-yl-1-[1-(pyridin-3-ylmethyl)piperidin-4-yl]-1H-pyrazolo[3,4-d]pyrimidin-6-yl}phenyl)-3-phenylurea;
1-[4-(1-{1-[(6-fluoropyridin-3-yl)methyl]piperidin-4-yl}-4-morpholin-4-yl-1H-pyrazolo[3,4-d]pyrimidin-6-yl)phenyl]-3-methylurea;
1-[4-(1-{1-[(6-methoxypyridin-3-yl)methyl]piperidin-4-yl}-4-morpholin-4-yl-1H-pyrazolo[3,4-d]pyrimidin-6-yl)phenyl]-3-methylurea;
1-[4-(1-{1-[(4-methoxypyridin-3-yl)methyl]piperidin-4-yl}-4-morpholin-4-yl-1H-pyrazolo[3,4-d]pyrimidin-6-yl)phenyl]-3-methylurea
1-methyl-3-(4-{1-[1-(4-methylbenzoyl)piperidin-4-yl]-4-morpholin-4-yl-1H-pyrazolo[3,4-d]pyrimidin-6-yl}phenyl)urea
Tert-butyl 4-(6-{4-[(methylcarbamoyl)amino]phenyl}-4-morpholin-4-yl-1H-pyrazolo[3,4-d]pyrimidin-1-yl)piperidine-1-carboxylate
1-[4-(1-{1-[(5-fluoropyridin-3-yl)methyl]piperidin-4-yl}-4-morpholin-4-yl-1H-pyrazolo[3,4-d]pyrimidin-6-yl)phenyl]-3-methylurea
1-(2-hydroxyethyl)-3-(4-{4-morpholin-4-yl-1-[1-(pyridin-3-ylmethyl)piperidin-4-yl-1H-pyrazolo[3,4-d]pyrimidin-6-yl}phenyl)urea
1-methyl-3-(4-{4-morpholin-4-yl-1-[1-(pyrazin-2-ylcarbonyl)piperidin-4-yl]-1H-pyrazolo[3,4-d]pyrimidin-6-yl}phenyl)urea
1-[4-(1-{1-[(6-chloropyridin-3-yl)carbonyl]piperidin-4-yl}-4-morpholin-4-yl-1H-pyrazolo[3,4-d]pyrimidin-6-yl)phenyl]-3-methylurea
1-{4-[1-(1-benzylpiperidin-4-yl)-4-morpholin-4-yl-1H-pyrazolo[3,4-d]pyrimidin-6-yl]phenyl}-3-(1H-imidazol-2-yl)urea
1-(4-{1-[1-(4-methoxybenzoyl)piperidin-4-yl]-4-morpholin-4-yl-1H-pyrazolo[3,4-d]pyrimidin-6-yl}phenyl)-3-methylurea
1-methyl-3-[4-(4-morpholin-4-yl-1-{1-[(1-oxidopyridin-3-yl)carbonyl]piperidin-4-yl}-1H-pyrazolo[3,4-d]pyrimidin-6-yl)phenyl]urea;
1-(4-{1-[1-(4-fluorobenzoyl)piperidin-4-yl]-4-morpholin-4-yl-1H-pyrazolo[3,4-d]pyrimidin-6-yl}phenyl)-3-methylurea
1-{4-[1-(1-benzylpiperidin-4-yl)-4-morpholin-4-yl-1H-pyrazolo[3,4-d]pyrimidin-6-yl]phenyl}-3-ethylurea
1-methyl-3-{4-[4-morpholin-4-yl-1-(tetrahydro-2H-pyran-4-yl)-1H-pyrazolo[3,4-d]pyrimidin-6-yl]phenyl}urea
1-(4-{1-[1-(3-chlorobenzyl)piperidin-4-yl]-4-morpholin-4-yl-1H-pyrazolo[3,4-d]pyrimidin-6-yl}phenyl)-3-methylurea
1-cyclopropyl-3-(4-{4-morpholin-4-yl-1-[1-(pyridin-3-ylmethyl)piperidin-4-yl]-1H-pyrazolo[3,4-d]pyrimidin-6-yl}phenyl)urea
1-{4-[1-(1-benzylpiperidin-4-yl)-4-morpholin-4-yl-1H-pyrazolo[3,4-d]pyrimidin-6-yl]phenyl}-3-pyridin-3-ylurea
1-(2-fluoroethyl)-3-(4-{4-morpholin-4-yl-1-[1-(pyridin-3-ylmethyl)piperidin-4-yl]-1H-pyrazolo[3,4-d]pyrimidin-6-yl}phenyl)urea
1-methyl-3-(4-{4-morpholin-4-yl-1-[1-(pyridin-3-ylcarbonyl)piperidin-4-yl]-1H-pyrazolo[3,4-d]pyrimidin-6-yl}phenyl)urea
1-(2-fluoroethyl)-3-[4-(4-morpholin-4-yl-1-phenyl-1H-pyrazolo[3,4-d]pyrimidin-6-yl)phenyl]urea
and 1-(4-{1-[1-(2-hydroxybenzyl)piperidin-4-yl]-4-morpholin-4-yl-1H-pyrazolo[3,4-d]pyrimidin-6-yl}phenyl)-3-methylurea.

26. A compound selected from the group consisting of
1-{1-[(2-chloropyridin-3-yl)carbonyl]piperidin-4-yl}-6-(1H-indol-5-yl)-4-yl-1H-pyrazolo[3,4-d]pyrimidine
1-(4-{4-morpholin-4-yl-1-[1-(pyridin-3-ylmethyl)piperidin-4-yl]-1H-pyrazolo[3,4-d]pyrimidin-6-yl}phenyl)-3-pyridin-2-ylurea
1-(4-{1-[1-(3-methoxybenzyl)piperidin-4-yl]-4-morpholin-4-yl-1H-pyrazolo[3,4-d]pyrimidin-6-yl}phenyl)-3-methylurea
1-butyl-3-(4-{4-morpholin-4-yl-1-[1-(pyridin-3-ylcarbonyl)piperidin-4-yl]-1H-pyrazolo[3,4-d]pyrimidin-6-yl}phenyl)urea
1-(4-{1-[1-(4-bromobenzoyl)piperidin-4-yl]-4-morpholin-4-yl-1H-pyrazolo[3,4-d]pyrimidin-6-yl}phenyl)-3-methylurea
1-(2-fluoro-4-{4-morpholin-4-yl-1-[1-(pyridin-3-ylmethyl)piperidin-4-yl]-1H-pyrazolo[3,4-d]pyrimidin-6-yl}phenyl)-3-methylurea
1-(4-{1-[1-(4-chlorobenzyl)piperidin-4-yl]-4-morpholin-4-yl-1H-pyrazolo[3,4-d]pyrimidin-6-yl}phenyl)-3-methylurea
N,N-dimethyl-4-(6-{4-[(methylcarbamoyl)amino]phenyl}-4-morpholin-4-yl-1H-pyrazolo[3,4-d]pyrimidin-1-yl)piperidine-1-carboxamide
1-(4-{1-[1-(4-chlorobenzoyl)piperidin-4-yl]-4-morpholin-4-yl-1H-pyrazolo[3,4-d]pyrimidin-6-yl}phenyl)-3-methylurea
1-{4-[1-(1-benzoylpiperidin-4-yl)-4-morpholin-4-yl-1H-pyrazolo[3,4-d]pyrimidin-6-yl]phenyl}-3-[2-(methylamino)ethyl]urea;
1-methyl-3-[4-(4-morpholin-4-yl-1-piperidin-4-yl-1H-pyrazolo[3,4-d]pyrimidin-6-yl)phenyl]urea
methyl (4-{4-morpholin-4-yl-1-[1-(pyridin-3-ylcarbonyl)piperidin-4-yl]-1H-pyrazolo [3,4-d]pyrimidin-6-yl}phenyl)carbamate
1-{4-[1-(1-benzylpiperidin-4-yl)-4-morpholin-4-yl-1H-pyrazolo[3,4-d]pyrimidin-6-yl]phenyl}-3-(cyclopropylmethyl)urea
methyl (4-{4-morpholin-4-yl-1-[1-(pyridin-3-ylmethyl)piperidin-4-yl]-1H-pyrazolo[3,4-d]pyrimidin-6-yl}phenyl)carbamate
and methyl (4-{4-morpholin-4-yl-1-[1-(pyridin-3-ylmethyl)piperidin-4-yl]-1H-pyrazolo[3,4-d]pyrimidin-6-yl}phenyl)carbamate.

27. A compound of the Formula IIc: 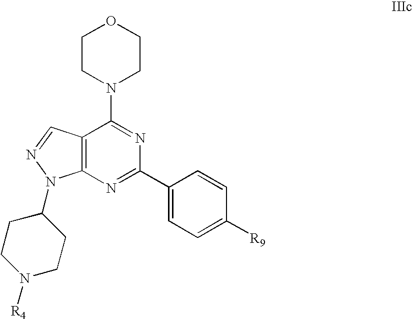
or a pharmaceutically acceptable salt or tautomer thereof, wherein
R₄ is selected from:
a) hydrogen;
b) C₁-C₈acyl, wherein the C₁-C₈acyl is optionally substituted with from 1 to 3 substituents independently selected from:
(i) hydroxyl,
(ii) CN,
(iii) C₁-C₆alkoxy,
(iv) C₁-C₆alkyl,
(v) C₁-C₈acyl,
(vi) NH₂,
(vii) (C₁-C₆alkyl)amino,
(viii) di(C₁-C₆alkyl)amino,
(ix) CO₂H,
(x) (C₁-C₆alkoxy)carbonyl,
(xi) C₁-C₆perfluoroalkyl,
(xii) and halogen;
c) C₁-C₆alkyl optionally substituted with from 1 to 3 substituents independently selected from:
(i) C₃-C₈cycloalkyl,
(ii) C₁-C₆alkoxy,
(iii) C₁-C₈acyl,
(iv) CN,
(v) (C₁-C₆alkoxy)carbonyl,
(vi) CO₂H,
(vii) hydroxyl,
(viii) C₁-C₉heterocycle,
(ix) and H₂NC(O)—;
d) C₁-C₆perfluoroalkyl;
e) C₂-C₆alkenyl;
f) heteroaryl(C₁-C₆alkyl) wherein the ring portion of the heteroaryl(C₁-C₆alkyl) group is optionally substituted with from 1 to 3 substituents independently selected from:
(i) C₁-C₆alkylC(O)NH—,
(ii) C₁-C₆alkoxy,
(iii) halogen,
(iv) NH₂,
(v) and C₁-C₆alkyl;
g) (C₆-C₁₄aryl)alkyl, wherein the ring portion of the (C₆-C₁₄aryl)alkyl group is optionally substituted by 1 to 3 substituents independently selected from:
(i) halogen,
(ii) C₁-C₆alkyl,
(iii) NH₂,
(iv) (C₁-C₆alkyl)amino,
(v) di(C₁-C₆alkyl)amino,
(vi) hydroxyl,
(vii) C₁-C₆alkoxy,
(viii) C₁-C₈acyl,
(ix) and C₁-C₉heteroaryl;
h) HC(O)—;
i) C₁-C₆perfluoroalkyl;
j) —S(O)_{q}—(C₁-C₆alkyl);
k) —S(O)_{q}-aryl;
l) R¹⁹R²⁰NC(O);
m) (C₁-C₉heteroaryl)-NH—C(S)—o;
n) (C₁-C₆alkyl)-NH—C(S)-;
o) (C₁-C₆alkyl)-S—C(O)—;
p) (C₆-C₁₄aryloxy)carbonyl;
q) (C₂-C₆alkenyloxy)carbonyl;
r) (C₂-C₆alkynyloxy)carbonyl;
s) and (C₁-C₆alkoxy)carbonyl optionally substituted with from 1 to 3 substituents independently selected from:
(i) C₁-C₆alkoxy,
(ii) halogen,
(iii) C₆-C₁₄aryl,
(iv) NH₂,
(v) (C₁-C₆alkyl)amino-,
(vi) di(C₁-C₆alkyl)amino-,
(vii) and C₁-C₆alkyl; 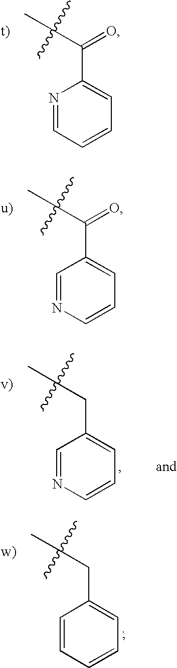
R₉ is —NHC(O)NR₁₀R₁₁, or —NHC(O)OR₁₂;
R₁₀ and R₁₁ are each independently —H, —OH, C₁-C₆alkoxy, C₆-C₁₄aryl, C₁-C₉heteroaryl, —C₃-C₈carbocycle, or —C₁-C₆alkyl; or R₁₀ and R₁₁ when taken together with the nitrogen to which they are attached to form a 3- to 7-membered nitrogen containing heterocycle wherein up to two of the carbon atoms of the heterocycle may be substituted with —N(R₁₅)—, —O—, or —S(O)ₙ;
R₁₂ is C₁-C₆alkyl, C₁-C₆hydroxylalkyl, or C₆-C₁₄aryl; and
R₁₅ is hydrogen, C₁-C₆alkyl, C₃-C₈carbocycle, C₆-C₁₄aryl, C₁-C₉heteroaryl, (C₁-C₆alkyl)amino, or substituted (C₆-C₁₄aryl)amino;
n is 0, 1, or 2;
q is 1 or 2;
R¹⁹ and R²⁰ are each independently:
a) H;
b) C₁-C₆alkyl optionally substituted with a substituent selected from:
(i) C₁-C₆alkylC(O)NH—,
(ii) NH₂,
(iii) (C₁-C₆alkyl)amino, and
(iv) di(C₁-C₆alkyl)amino;
c) C₃-C₈cycloalkyl;
d) C₆-C₁₄aryl optionally substituted with a substituent selected from:
(i) halogen,
(ii) and monocyclic C₁-C₆heterocycle wherein the monocyclic C₁-C₆heterocycle is optionally substituted with (C₁-C₆alkoxy)carbonyl;
e) C₁-C₉heteroaryl;
f) heteroaryl(C₁-C₆alkyl);
g) heterocyclyl(C₁-C₆alkyl);
h) (C₆-C₁₄aryl)alkyl, wherein the chain portion of the (C₆-C₁₄aryl)alkyl group is optionally substituted by a hydroxyl;
i) or monocyclic C₁-C₆heterocycle optionally substituted with a (C₁-C₆alkoxy)carbonyl;
or R¹⁹ and R²⁰ when taken together with the nitrogen to which they are attached optionally form a 3- to 7-membered nitrogen-containing heterocycle wherein up to two of the carbon atoms of the heterocycle are optionally replaced with —N(H)—, —N(C₁-C₆alkyl)-, —N(C₆-C₁₄aryl)-, or —O—, and wherein the nitrogen-containing heterocycle is optionally substituted by a C₁-C₆alkyl; C₆-C₁₄aryl, (C₁-C₆alkoxy)C(O)NH—, or C₁-C₉heterocycle.

28. The compound of claim 27, wherein R₄ is (C₁-C₆alkoxy)carbonyl optionally independently substituted with from 1 to 3 substituents as specified in claim 37.

29. The compound of claim 28, wherein R₄ is (C₁-C₆alkoxy)carbonyl.

30. The compound of claim 29, wherein R₄ is ethoxycarbonyl.

31. The compound of claim 27, wherein R₄ is 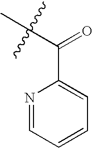
or a pharmaceutically acceptable salt thereof.

32. The compound of claim 27, wherein, R₄ is 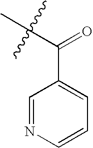
or a pharmaceutically acceptable salt thereof.

33. The compound of claim 27, wherein R₄ is 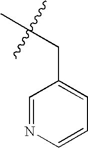
or a pharmaceutically acceptable salt thereof.

34. The compound of claim 37, wherein R₄ is 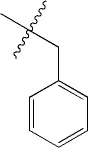
or a pharmaceutically acceptable salt thereof.

35. The compound of claim 27, wherein R₁₉ is hydrogen.

36. The compound of claim 27, wherein R₂₀ is C₁-C₆alkyl.

37. The compound of claim 27, wherein R₂₀ is C₆-C₁₄aryl.

38. The compound of claim 27, wherein R¹⁹ and R²⁰ when taken together with the nitrogen to which they are attached optionally form a 3- to 7-membered nitrogen-containing heterocycle wherein up to two of the carbon atoms of the heterocycle are optionally replaced with —N(H)—, —N(C₁-C₆alkyl)-, —N(C₆-C₁₄aryl)-, or —O—, and wherein the nitrogen-containing heterocycle is optionally substituted by a C₁-C₆alkyl; C₆-C₁₄aryl, (C₁-C₆alkoxy)C(O)NH—, or C₁-C₉heterocycle.

39. The compound of claim 27, wherein R₉ is —NHC(O)NR₁₀R₁₁.

40. The compound of claim 39, wherein R₁₀ is hydrogen.

41. The compound of claim 39, wherein R₁₁ is C₁-C₉heteroaryl or C₁-C₆alkyl.

42. The compound of claim 41, wherein R₁₁ is C₁-C₆alkyl.

43. The compound of claim 42, wherein R₁₁ is ethyl.

44. The compound of claim 41, wherein R₁₁ is C₁-C₉heteroaryl.

45. The compound of claim 44, wherein R₁₁ is pyridyl.

46. The compound of claim 45, wherein R₁₁ is 4-pyridyl.

47. The compound of claim 27, wherein R₉ is —NHC(O)OR₁₂.

48. The compound of claim 47, wherein R₁₂ is C₁-C₆alkyl or C₁-C₆hydroxylalkyl.

49. The compound of claim 48, wherein R₁₂ is C₁-C₆hydroxylalkyl.

50. The compound of claim 49, wherein R₁₂ is hydroxylethyl.

51. The compound of claim 48, wherein R₁₂ is propyl.

52. A compound selected from the group consisting of:
methyl{3-[1-(1-benzylpiperidin-4-yl)-4-morpholin-4-yl-1H-pyrazolo[3,4-d]pyrimidin-6-yl]phenyl}carbamate;
N-{3-[1-(1-benzylpiperidin-4-yl)-4-morpholin-4-yl-1H-pyrazolo[3,4-d]pyrimidin-6-yl]phenyl}-N′-methylurea;
N-{3-[1-(1-benzylpiperidin-4-yl)-4-morpholin-4-yl-1H-pyrazolo[3,4-d]pyrimidin-6-yl]phenyl}urea;
3-[1-(1-benzylpiperidin-4-yl)-4-morpholin-4-yl-1H-pyrazolo[3,4-d]pyrimidin-6-yl]quinoline;
N-{4-[1-(1-benzylpiperidin-4-yl)-4-morpholin-4-yl-1H-pyrazolo[3,4-d]pyrimidin-6-yl]phenyl}formamide;
4-[1-(1-benzylpiperidin-4-yl)-4-morpholin-4-yl-1H-pyrazolo[3,4-d]pyrimidin-6-yl]phenol;
4-{4-[6-(1H-indol-5-yl)-4-morpholin-4-yl-1H-pyrazolo[3,4-d]pyrimidin-1-yl]piperidin-1-yl}-N,N-dimethyl-4-oxobut-2-en-1-amine;
6-(1H-indol-5-yl)-1-(1-isopropylpiperidin-4-yl)-4-morpholin-4-yl-1H-pyrazolo[3,4-d]pyrimidine;
6-(1H-indol-5-yl)-4-morpholin-4-yl-1-[1-(2-phenylethyl)piperidin-4-yl]-1H-pyrazolo[3,4-d]pyrimidine;
6-(1H-indol-5-yl)-4-morpholin-4-yl-1-[1-(1-phenylethyl)piperidin-4-yl]-1H-pyrazolo[3,4-d]pyrimidine;
6-(1H-indol-5-yl) 1-[1-(2-methoxyethyl)piperidin-4-yl]-4-morpholin-4-yl-1H-pyrazolo[3,4-d]pyrimidine;
6-(1H-indol-5-yl)-4-morpholin-4-yl-1-[1-(phenylacetyl)piperidin-4-yl]-1H-pyrazolo[3,4-d]pyrimidine;
phenyl 4-[6-(1H-indol-5-yl)-4-morpholin-4-yl-1H-pyrazolo[3,4-d]pyrimidin-1-yl]piperidine-1-carboxylate;
methyl 4-[6-(1H-indol-5-yl)-4-morpholin-4-yl-1H-pyrazolo[3,4-d]pyrimidin-1-yl]piperidine-1-carboxylate;
2-{4-[6-(1H-indol-5-yl)-4-morpholin-4-yl-1H-pyrazolo[3,4-d]pyrimidin-1-yl]piperidin-1-yl}acetamide;
2-{4-[6-(1H-indol-5-yl)-4-morpholin-4-yl-1H-pyrazolo[3,4-d]pyrimidin-1-yl]piperidin-1yl}ethanol;
3-{4-[6-(1H-indol-5-yl)-4-morpholin-4-yl-1H-pyrazolo[3,4-d]pyrimidin-1-yl]piperidin-1-yl}propan-1-ol;
1-{4-[1-(1-benzylpiperidin-4-yl)-4-morpholin-4-yl-1H-pyrazolo[3,4-d]pyrimidin-6-yl]phenyl}urea;
1-{4-[1-(1-benzylpiperidin-4-yl)-4-morpholin-4-yl-1H-pyrazolo[3,4-d]pyrimidin-6-yl]phenyl}-3-ethylurea;
1-{4-[1-(1-benzylpiperidin-4-yl)-4-morpholin-4-yl-1H-pyrazolo[3,4-d]pyrimidin-6-yl]phenyl}-3-propylurea;
propyl{4-[1-(1-benzylpiperidin-4-yl)-4-morpholin-4-yl-1H-pyrazolo[3,4-d]pyrimidin-6-yl]phenyl}carbamate;
1-{4-[1-(1-benzylpiperidin-4-yl)-4-morpholin-4-yl-1H-pyrazolo[3,4-d]pyrimidin-6-yl]phenyl}-3-isopropylurea;
1-{4-[1-(1-benzylpiperidin-4-yl)-4-morpholin-4-yl-1H-pyrazolo[3,4-d]pyrimidin-6-yl]phenyl}-3-phenylurea;
1-benzyl-3-{4-[1-(1-benzylpiperidin-4-yl)-4-morpholin-4-yl-1H-pyrazolo[3,4-d]pyrimidin-6-yl]phenyl}urea;
1-{4-[1-(1-benzylpiperidin-4-yl)-4-morpholin-4-yl-1H-pyrazolo[3,4-d]pyrimidin-6-yl]phenyl}-3-(2-phenylethyl)urea;
1-{4-[1-(1-benzylpiperidin-4-yl)-4-morpholin-4-yl-1H-pyrazolo[3,4-d]pyrimidin-6-yl]phenyl}-3-(3-phenylpropyl)urea;
1-{4-[1-(1-benzylpiperidin-4-yl)-4-morpholin-4-yl-1H-pyrazolo[3,4-d]pyrimidin-6-yl]phenyl}-3-pyridin-3-ylurea;
1-{4-[1-(1-benzylpiperidin-4-yl)-4-morpholin-4-yl-1H-pyrazolo[3,4-d]pyrimidin-6-yl]phenyl}-3-(cyclopropylmethyl)urea;
1-allyl-3-{4-[1-(1-benzylpiperidin-4-yl)-4-morpholin-4-yl-1H-pyrazolo[3,4-d]pyrimidin-6-yl]phenyl}urea;
1-{4-[1-(1-benzylpiperidin-4-yl)-4-morpholin-4-yl-1H-pyrazolo[3,4-d]pyrimidin-6-yl]phenyl}-3-(2-hydroxyethyl)urea;
1-{4-[1-(1-benzylpiperidin-4-yl)-4-morpholin-4-yl-1H-pyrazolo[3,4-d]pyrimidin-6-yl]phenyl}-3-(2-methoxyethyl)urea;
1-(2-aminoethyl)-3-{4-[1-(1-benzylpiperidin-4-yl)-4-morpholin-4-yl-1H-pyrazolo[3,4-d]pyrimidin-6-yl]phenyl}urea;
1-{4-[1-(1-benzylpiperidin-4-yl)-4-morpholin-4-yl-1H-pyrazolo[3,4-d]pyrimidin-6-yl]phenyl}-3-[2-(dimethylamino)ethyl]urea;
1-{4-[1-(1-benzylpiperidin-4-yl)-4-morpholin-4-yl-1H-pyrazolo[3,4-d]pyrimidin-6-yl]phenyl}-3-(3-hydroxypropyl)urea;
1-{4-[1-(1-benzylpiperidin-4-yl)-4-morpholin-4-yl-1H-pyrazolo[3,4-d]pyrimidin-6-yl]phenyl}-3-(3-methoxypropyl)urea;
1-{4-[1-(1-benzylpiperidin-4-yl)-4-morpholin-4-yl-1H-pyrazolo[3,4-d]pyrimidin-6-yl]phenyl}-3-[3-(dimethylamino)propyl]urea;
1-{4-[1-(1-benzylpiperidin-4-yl)-4-morpholin-4-yl-1H-pyrazolo[3,4-d]pyrimidin-6-yl]phenyl}-3-(1-methylpiperidin-4-yl)urea;
4-[6-(1H-indol-5-yl)-4-morpholin-4-yl-1H-pyrazolo[3,4-d]pyrimidin-1-yl]piperidine carbaldehyde;
3-methoxy-N-{4-[4-morpholin-4-yl-1-(tetrahydro-2H-pyran-2-yl)-1H-pyrazolo[3,4-d]pyrimidin-6-yl]phenyl}benzamide;
methyl{4-[4-morpholin-4-yl-1-(tetrahydro-2H-pyran-2-yl)-1H-pyrazolo[3,4-d]pyrimidin-6-yl]phenyl}carbamate;
N2,N2-dimethyl-N-{4-[4-morpholin-4-yl-1-(tetrahydro-2H-pyran-2-yl)-1H-pyrazolo[3,4-d]pyrimidin-6-yl]phenyl}glycinamide;
2-[4-(dimethylamino)phenyl]-N-{4-[4-morpholin-4-yl-1-(tetrahydro-2H-pyran-2-yl)-1H-pyrazolo[3,4-d]pyrimidin-6-yl]phenyl}acetamide;
3-methoxy-N-[4-(4-morpholin-4-yl-1H-pyrazolo[3,4-d]pyrimidin-6-yl)phenyl]benzamide;
N-[4-(4-morpholin-4-yl-1H-pyrazolo[3,4-d]pyrimidin-6-yl)phenyl]nicotinamide;
methyl[4-(4-morpholin-4-yl-1H-pyrazolo[3,4-d]pyrimidin-6-yl)phenyl]carbamate;
N-[4-(4-morpholin-4-yl-1H-pyrazolo[3,4-d]pyrimidin-6-yl)phenyl]acrylamide;
N2,N2-dimethyl-N-[4-(4-morpholin-4-yl-1H-pyrazolo[3,4-d]pyrimidin-6-yl)phenyl]glycinamide;
N-[4-(4-morpholin-4-yl-1H-pyrazolo[3,4-d]pyrimidin-6-yl)phenyl]glycinamide;
N-[4-(4-morpholin-4-yl-1H-pyrazolo[3,4-d]pyrimidin-6-yl)phenyl]-b-alaninamide;
1-methyl-N-[4-(4-morpholin-4-yl-1H-pyrazolo[3,4-d]pyrimidin-6-yl)phenyl]piperidine-4-carboxamide;
4-(4-morpholin-4-yl-1H-pyrazolo[3,4-d]pyrimidin-6-yl)aniline;
1-{4-[1-(1-benzylpiperidin-4-yl)-4-morpholin-4-yl-1H-pyrazolo[3,4-d]pyrimidin-6-yl]phenyl}-3-methoxyurea;
1-{4-[1-(1-benzylpiperidin-4-yl)-4-morpholin-4-yl-1H-pyrazolo[3,4-d]pyrimidin-6-yl]phenyl}-3-ethoxyurea;
1-{4-[1-(1-benzylpiperidin-4-yl)-4-morpholin-4-yl-1H-pyrazolo[3,4-d]pyrimidin-6-yl]phenyl}-3-(2-fluoroethyl)urea;
1-{4-[1-(1-benzylpiperidin-4-yl)-4-morpholin-4-yl-1H-pyrazolo[3,4-d]pyrimidin-6-yl]phenyl}-3-(2,2,2-trifluoroethyl)urea;
N-{4-[1-(1-benzylpiperidin-4-yl)-4-morpholin-4-yl-1H-pyrazolo[3,4-d]pyrimidin-6-yl]phenyl}-2,2-dimethylhydrazinecarboxamide;
1-{4-[1-(1-benzylpiperidin-4-yl)-4-morpholin-4-yl-1H-pyrazolo[3,4-d]pyrimidin-6-yl]phenyl}-3-pyrrolidin-1-ylurea;
N-[4-(4-morpholin-4-yl-1-phenyl-1H-pyrazolo[3,4-d]pyrimidin-6-yl)phenyl]hydrazinecarboxamide;
1-hydroxy-3-[4-(4-morpholin-4-yl-1-phenyl-1H-pyrazolo[3,4-d]pyrimidin-6-yl)phenyl]urea;
1-(2-fluoroethyl)-3-[4-(4-morpholin-4-yl-1-phenyl-1H-pyrazolo[3,4-d]pyrimidin-6-yl)phenyl]urea;
1-methoxy-3-[4-(4-morpholin-4-yl-1-phenyl-1H-pyrazolo[3,4-d]pyrimidin-6-yl)phenyl]urea;
1-(allyloxy)-3-[4-(4-morpholin-4-yl-1-phenyl-1H-pyrazolo[3,4-d]pyrimidin-6-yl)phenyl]urea;
1-methyl-3-[4-(4-morpholin-4-yl-1-phenyl-1H-pyrazolo[3,4-d]pyrimidin-6-yl)phenyl]urea;
N-{4-[1-(1-benzylpiperidin-4-yl)-4-morpholin-4-yl-1H-pyrazolo[3,4-d]pyrimidin-6-yl]phenyl}-2,2,2-trifluoroacetamide;
1-{4-[1-(1-benzylpiperidin-4-yl)-4-morpholin-4-yl-1H-pyrazolo[3,4-d]pyrimidin-6-yl]phenyl}-3-[2-(methylamino)ethyl]urea;
1-(4-{4-morpholin-4-yl-1-[1-(pyridin-3-ylcarbonyl)piperidin-4-yl]-1H-pyrazolo[3,4-d]pyrimidin-6-yl}phenyl)urea;
1-(4-{4-morpholin-4-yl-1-[1-(pyridin-3-ylcarbonyl)piperidin-4-yl]-1H-pyrazolo[3,4-d]pyrimidin-6-yl}phenyl)-3-pyridin-3-ylurea;
1-(2-fluoroethyl)-3-(4-{4-morpholin-4-yl-1-[1-(pyridin-3-ylcarbonyl)piperidin-4-yl]-1H-pyrazolo[3,4-d]pyrimidin-6-yl}phenyl)urea;
1-(2-hydroxyethyl)-3-(4-{4-morpholin-4-yl-1-[1-(pyridin-3-ylcarbonyl)piperidin-4-yl]-1H-pyrazolo[3,4-d]pyrimidin-6-yl}phenyl)urea;
1-hydroxy-3-(4-{4-morpholin-4-yl-1-[1-(pyridin-3-ylcarbonyl)piperidin-4-yl]-1H-pyrazolo[3,4-d]pyrimidin-6-yl}phenyl)urea;
N-(4-{4-morpholin-4-yl-1-[1-(pyridin-3-ylcarbonyl)piperidin-4-yl]-1H-pyrazolo[3,4-d]pyrimidin-6-yl}phenyl)hydrazinecarboxamide;
1-ethyl-3-(4-{4-morpholin-4-yl-1-[1-(pyridin-3-ylcarbonyl)piperidin-4-yl]-1H-pyrazolo[3,4-d]pyrimidin-6-yl}phenyl)urea;
1-methoxy-3-(4-{4-morpholin-4-yl-1-[1-(pyridin-3-ylcarbonyl)piperidin-4-yl]-1H-pyrazolo[3,4-d]pyrimidin-6-yl}phenyl)urea;
N-{4-[1-(1-benzylpiperidin-4-yl)-4-morpholin-4-yl-1H-pyrazolo[3,4-d]pyrimidin-6-yl]phenyl}hydrazinecarboxamide;
1-{4-[1-(1-benzylpiperidin-4-yl)-4-morpholin-4-yl-1H-pyrazolo[3,4-d]pyrimidin-6-yl]phenyl}-3-hydroxyurea;
1-{4-[1-(1-benzylpiperidin-4-yl)-4-morpholin-4-yl-1H-pyrazolo[3,4-d]pyrimidin-6-yl]phenyl}-3-cyclopropylurea;
1-{4-[1-(1-benzylpiperidin-4-yl)-4-morpholin-4-yl-1H-pyrazolo[3,4-d]pyrimidin-6-yl]phenyl}-3-prop-2-yn-1-ylurea;
1-(4-{4-morpholin-4-yl-1-[1-(pyridin-3-ylmethyl)piperidin-4-yl]-1H-pyrazolo[3,4-d]pyrimidin-6-yl}phenyl)urea;
1-(4-{4-morpholin-4-yl-1-[1-(pyridin-3-ylmethyl)piperidin-4-yl]-1H-pyrazolo[3,4-d]pyrimidin-6-yl}phenyl)-3-pyridin-3-ylurea;
1-(2-fluoroethyl)-3-(4-{4-morpholin-4-yl-1-[1-(pyridin-3-ylmethyl)piperidin-4-yl]-1H-pyrazolo[3,4-d]pyrimidin-6-yl}phenyl)urea;
1-(2-hydroxyethyl)-3-(4-{4-morpholin-4-yl-1-[1-(pyridin-3-ylmethyl)piperidin-4-yl]-1H-pyrazolo[3,4-d]pyrimidin-6-yl}phenyl)urea;
1-hydroxy-3-(4-{4-morpholin-4-yl-1-[1-(pyridin-3-ylmethyl)piperidin-4-yl]-1H-pyrazolo[3,4-d]pyrimidin-6-yl}phenyl)urea;
1-ethyl-3-(4-{4-morpholin-4-yl-1-[1-(pyridin-3-ylmethyl)piperidin-4-yl]-1H-pyrazolo[3,4-d]pyrimidin-6-yl}phenyl)urea;
1-methoxy-3-(4-{4-morpholin-4-yl-1-[1-(pyridin-3-ylmethyl)piperidin-4-yl]-1H-pyrazolo[3,4-d]pyrimidin-6-yl}phenyl)urea;
4-[1-(1,4-dioxaspiro[4.5]dec-8-yl)-4-morpholin-4-yl-1H-pyrazolo[3,4-d]pyrimidin-6-yl]aniline;
1-{4-[1-(1,4-dioxaspiro[4.5]dec-8-yl)-4-morpholin-4-yl-1H-pyrazolo[3,4-d]pyrimidin-6-yl]phenyl}-3-methylurea;
1-methyl-3-{4-[4-morpholin-4-yl-1-(4-oxocyclohexyl)-1H-pyrazolo[3,4-d]pyrimidin-6-yl]phenyl}urea;
1-{4-[1-(4-hydroxycyclohexyl)-4-morpholin-4-yl-1H-pyrazolo[3,4-d]pyrimidin-6-yl]phenyl}-3-methylurea;
1-{4-[1-(1-benzylpiperidin-4-yl)-4-morpholin-4-yl-1H-pyrazolo[3,4-d]pyrimidin-6-yl]phenyl}-3-methylthiourea;
1-{4-[1-(1-benzylpiperidin-4-yl)-4-morpholin-4-yl-1H-pyrazolo[3,4-d]pyrimidin-6-yl]phenyl}-3-(1H-imidazol-2-yl)urea;
5-[1-(1-benzylpiperidin-4-yl)-4-morpholin-4-yl-1H-pyrazolo[3,4-d]pyrimidin-6-yl]-1,3-dihydro-2H-benzimidazol-2-one;
1-methyl-3-[4-(4-morpholin-4-yl-1-{1-[(1-oxidopyridin-3-yl)carbonyl]piperidin-4-yl}-1H-pyrazolo[3,4-d]pyrimidin-6-yl)phenyl]urea;
1-methyl-3-[4-(1-{1-[(2-methylpyridin-3-yl)carbonyl]piperidin-4-yl}-4-morpholin-4-yl-1H-pyrazolo[3,4-d]pyrimidin-6-yl)phenyl]urea;
1-[4-(1-{1-[(6-methoxypyridin-3-yl)methyl]piperidin-4-yl}-4-morpholin-4-yl-1H-pyrazolo[3,4-d]pyrimidin-6-yl)phenyl]-3-methylurea;
1-methyl-3-(4-{4-morpholin-4-yl-1-[1-(pyridin-2-ylmethyl)piperidin-4-yl]-1H-pyrazolo[3,4-d]pyrimidin-6-yl}phenyl)urea;
2-[4-(6-{4-[(methylcarbamoyl)amino]phenyl}-4-morpholin-4-yl-1H-pyrazolo[3,4-d]pyrimidin-1-yl)piperidin-1-yl]acetamide;
1-methyl-3-(4-{4-morpholin-4-yl-1-[1-(2-oxo-2-phenylethyl)piperidin-4-yl]-1H-pyrazolo[3,4-d]pyrimidin-6-yl}phenyl)urea;
1-methyl-3-[4-(1-{1-[(4-methylpiperazin-1-yl)carbonyl]piperidin-4-yl}-4-morpholin-4-yl-1H-pyrazolo[3,4-d]pyrimidin-6-yl)phenyl]urea;
4-(6-{4-[(methylcarbamoyl)amino]phenyl}-4-morpholin-4-yl-1H-pyrazolo[3,4-d]pyrimidin-1-yl)-N-pyridin-3-ylpiperidine-1-carboxamide;
1-{4-[1-(1-benzoylpiperidin-4-yl)-4-morpholin-4-yl-1H-pyrazolo[3,4-d]pyrimidin-6-yl]phenyl}-3-methylurea;
1-{4-[1-(1-benzoylpiperidin-4-yl)-4-morpholin-4-yl-1H-pyrazolo[3,4-d]pyrimidin-6-yl]phenyl}-3-methylurea;
tert-butyl 4-(6-{4-[(methylcarbamoyl)amino]phenyl}-4-morpholin-4-yl-1H-pyrazolo[3,4-d]pyrimidin-1-yl)piperidine-1-carboxylate;
tert-butyl 4-(6-{4-[(methylcarbamoyl)amino]phenyl}-4-morpholin-4-yl-1H-pyrazolo[3,4-d]pyrimidin-1-yl)piperidine-1-carboxylate;
1-methyl-3-[4-(4-morpholin-4-yl-1-piperidin-4-yl-1H-pyrazolo[3,4-d]pyrimidin-6-yl)phenyl]urea;
1-methyl-3-[4-(4-morpholin-4-yl-1-piperidin-4-yl-1H-pyrazolo[3,4-d]pyrimidin-6-yl)phenyl]urea;
1-(4-{4-morpholin-4-yl-1-[1-(pyridin-3-ylmethyl)piperidin-4-yl]-1H-pyrazolo[3,4-d]pyrimidin-6-yl}phenyl)-3-phenylurea;
1-(4-{4-morpholin-4-yl-1-[1-(pyridin-3-ylmethyl)piperidin-4-yl]-1H-pyrazolo[3,4-d]pyrimidin-6-yl}phenyl)-3-pyridin-4-ylurea;
1-(4-{4-morpholin-4-yl-1-[1-(pyridin-3-ylmethyl)piperidin-4-yl]-1H-pyrazolo[3,4-d]pyrimidin-6-yl}phenyl)-3-pyridin-2-ylurea;
1-[2-(methylamino)ethyl]-3-(4-{4-morpholin-4-yl-1-[1-(pyridin-3-ylmethyl)piperidin-4-yl]-1H-pyrazolo[3,4-d]pyrimidin-6-yl}phenyl)urea;
1-(4-{4-morpholin-4-yl-1-[1-(pyridin-3-ylcarbonyl)piperidin-4-yl]-1H-pyrazolo[3,4-d]pyrimidin-6-yl}phenyl)-3-phenylurea;
1-(4-{4-morpholin-4-yl-1-[1-(pyridin-3-ylcarbonyl)piperidin-4-yl]-1H-pyrazolo[3,4-d]pyrimidin-6-yl}phenyl)-3-pyridin-4-ylurea;
1-(4-{4-morpholin-4-yl-1-[1-(pyridin-3-ylcarbonyl)piperidin-4-yl]-1H-pyrazolo[3,4-d]pyrimidin-6-yl}phenyl)-3-pyridin-2-ylurea;
1-[2-(methylamino)ethyl]-3-(4-{4-morpholin-4-yl-1-[1-(pyridin-3-ylcarbonyl)piperidin-4-yl]-1H-pyrazolo[3,4-d]pyrimidin-6-yl}phenyl)urea;
4-[1-(1-benzylpiperidin-4-yl)-4-morpholin-4-yl-1H-pyrazolo[3,4-d]pyrimidin-6-yl]-2-fluoroaniline;
1-{4-[1-(1-benzylpiperidin-4-yl)-4-morpholin-4-yl-1H-pyrazolo[3,4-d]pyrimidin-6-yl]-2-fluorophenyl}-3-methylurea;
1-{4-[1-(1-benzylpiperidin-4-yl)-4-morpholin-4-yl-1H-pyrazolo[3,4-d]pyrimidin-6-yl]-2-fluorophenyl}-3-ethylurea;
1-(2-fluoro-4-{4-morpholin-4-yl-1-[1-(pyridin-3-ylmethyl)piperidin-4-yl]-1H-pyrazolo[3,4-d]pyrimidin-6-yl}phenyl)-3-methylurea;
1-(2-fluoro-4-{4-morpholin-4-yl-1-[1-(pyridin-3-ylmethyl)piperidin-4-yl]-1H-pyrazolo[3,4-d]pyrimidin-6-yl}phenyl)-3-phenylurea;
1-(2-fluoro-4-{4-morpholin-4-yl-1-[1-(pyridin-3-ylmethyl)piperidin-4-yl]-1H-pyrazolo[3,4-d]pyrimidin-6-yl}phenyl)-3-pyridin-4-ylurea;
1-(4-{1-[1-(2-fluorobenzoyl)piperidin-4-yl]-4-morpholin-4-yl-1H-pyrazolo[3,4-d]pyrimidin-6-yl}phenyl)-3-methylurea;
1-(4-{1-[1-(2-chlorobenzoyl)piperidin-4-yl]-4-morpholin-4-yl-1H-pyrazolo[3,4-d]pyrimidin-6-yl}phenyl)-3-methylurea;
1-methyl-3-(4-{1-[1-(2-methylbenzoyl)piperidin-4-yl]-4-morpholin-4-yl-1H-pyrazolo[3,4-d]pyrimidin-6-yl}phenyl)urea;
1-(4-{1-[1-(3-fluorobenzoyl)piperidin-4-yl]-4-morpholin-4-yl-1H-pyrazolo[3,4-d]pyrimidin-6-yl}phenyl)-3-methylurea;
1-(4-{1-[1-(3-chlorobenzoyl)piperidin-4-yl]-4-morpholin-4-yl-1H-pyrazolo[3,4-d]pyrimidin-6-yl}phenyl)-3-methylurea;
1-methyl-3-[4-(4-morpholin-4-yl-1-{1-[3-(trifluoromethyl)benzoyl]piperidin-4-yl}-1H-pyrazolo[3,4-d]pyrimidin-6-yl)phenyl]urea;
1-(4-{1-[1-(4-bromobenzoyl)piperidin-4-yl]-4-morpholin-4-yl-1H-pyrazolo[3,4-d]pyrimidin-6-yl}phenyl)-3-methylurea;
1-(4-{1-[1-(4-fluorobenzoyl)piperidin-4-yl]-4-morpholin-4-yl-1H-pyrazolo[3,4-d]pyrimidin-6-yl}phenyl)-3-methylurea;
1-(4-{1-[1-(4-chlorobenzoyl)piperidin-4-yl]-4-morpholin-4-yl-1H-pyrazolo[3,4-d]pyrimidin-6-yl}phenyl)-3-methylurea;
1-(4-{1-[1-(4-methoxybenzoyl)piperidin-4-yl]-4-morpholin-4-yl-1H-pyrazolo[3,4-d]pyrimidin-6-yl}phenyl)-3-methylurea;
1-(4-{1-[1-(3-methoxybenzoyl)piperidin-4-yl]-4-morpholin-4-yl-1H-pyrazolo[3,4-d]pyrimidin-6-yl}phenyl)-3-methylurea;
1-(4-{1-[1-(2-methoxybenzoyl)piperidin-4-yl]-4-morpholin-4-yl-1H-pyrazolo[3,4-d]pyrimidin-6-yl}phenyl)-3-methylurea;
1-methyl-3-(4-{1-[1-(3-methylbenzoyl)piperidin-4-yl]-4-morpholin-4-yl-1H-pyrazolo[3,4-d]pyrimidin-6-yl}phenyl)urea;
1-methyl-3-(4-{1-[1-(4-methylbenzoyl)piperidin-4-yl]-4-morpholin-4-yl-1H-pyrazolo[3,4-d]pyrimidin-6-yl}phenyl)urea;
1-(4-{1-[1-(4-cyanobenzoyl)piperidin-4-yl]-4-morpholin-4-yl-1H-pyrazolo[3,4-d]pyrimidin-6-yl}phenyl)-3-methylurea;
2-{4-[1-(1-benzylpiperidin-4-yl)-4-morpholin-4-yl-1H-pyrazolo[3,4-d]pyrimidin-6-yl]phenyl}acetamide;
2-{4-[1-(1-benzylpiperidin-4-yl)-4-morpholin-4-yl-1H-pyrazolo[3,4-d]pyrimidin-6-yl]phenyl}-N-methylacetamide;
1-ethyl-3-(2-fluoro-4-{4-morpholin-4-yl-1-[1-(pyridin-3-ylmethyl)piperidin-4-yl]-1H-pyrazolo[3,4-d]pyrimidin-6-yl}phenyl)urea;
1-(2-fluoro-4-{4-morpholin-4-yl-1-[1-(pyridin-3-ylmethyl)piperidin-4-yl]-1H-pyrazolo[3,4-d]pyrimidin-6-yl}phenyl)-3-pyridin-3-ylurea;
1-(2-fluoroethyl)-3-(2-fluoro-4-{4-morpholin-4-yl-1-[1-(pyridin-3-ylmethyl)piperidin-4-yl]-1H-pyrazolo[3,4-d]pyrimidin-6-yl}phenyl)urea;
1-(4-{4-morpholin-4-yl-1-[1-(pyridin-3-ylmethyl)piperidin-4-yl]-1H-pyrazolo[3,4-d]pyrimidin-6-yl}phenyl)-3-(3-thienyl)urea;
1-(2-furylmethyl)-3-(4-{4-morpholin-4-yl-1-[1-(pyridin-3-ylmethyl)piperidin-4-yl]-1H-pyrazolo[3,4-d]pyrimidin-6-yl}phenyl)urea;
1-methyl-3-(4-{4-morpholin-4-yl-1-[1-(pyridin-3-ylmethyl)piperidin-4-yl]-1H-pyrazolo[3,4-d]pyrimidin-6-yl}phenyl)thiourea;
1-{4-[1-(1-benzoylpiperidin-4-yl)-4-morpholin-4-yl-1H-pyrazolo[3,4-d]pyrimidin-6-yl]phenyl}-3-phenylurea;
1-{4-[1-(1-benzoylpiperidin-4-yl)-4-morpholin-4-yl-1H-pyrazolo[3,4-d]pyrimidin-6-yl]phenyl}-3-pyridin-3-ylurea;
1-{4-[1-(1-benzoylpiperidin-4-yl)-4-morpholin-4-yl-1H-pyrazolo[3,4-d]pyrimidin-6-yl]phenyl}-3-(2-fluoroethyl)urea;
1-{4-[1-(1-benzoylpiperidin-4-yl)-4-morpholin-4-yl-1H-pyrazolo[3,4-d]pyrimidin-6-yl]phenyl}-3-ethylurea;
1-{4-[1-(1-benzoylpiperidin-4-yl)-4-morpholin-4-yl-1H-pyrazolo[3,4-d]pyrimidin-6-yl]phenyl}urea;
ethyl{4-[1-(1-benzoylpiperidin-4-yl)-4-morpholin-4-yl-1H-pyrazolo[3,4-d]pyrimidin-6-yl]phenyl}carbamate;
1-{4-[1-(1-benzoylpiperidin-4-yl)-4-morpholin-4-yl-1H-pyrazolo[3,4-d]pyrimidin-6-yl]phenyl}-3-pyridin-4-ylurea;
1-{4-[1-(1-benzoylpiperidin-4-yl)-4-morpholin-4-yl-1H-pyrazolo[3,4-d]pyrimidin-6-yl]phenyl}-3-(2-hydroxyethyl)urea;
1-{4-[1-(1-benzoylpiperidin-4-yl)-4-morpholin-4-yl-1H-pyrazolo[3,4-d]pyrimidin-6-yl]phenyl}-3-[2-(methylamino)ethyl]urea;
1-[4-(1-{1-[(6-fluoropyridin-3-yl)methyl]piperidin-4-yl}-4-morpholin-4-yl-1H-pyrazolo[3,4-d]pyrimidin-6-yl)phenyl]-3-methylurea;
1-[4-(1-{1-[(6-chloropyridin-3-yl)methyl]piperidin-4-yl}-4-morpholin-4-yl-1H-pyrazolo[3,4-d]pyrimidin-6-yl)phenyl]-3-methylurea;
1-[4-(1-{1-[(6-bromopyridin-3-yl)methyl]piperidin-4-yl}-4-morpholin-4-yl-1H-pyrazolo[3,4-d]pyrimidin-6-yl)phenyl]-3-methylurea;
1-[4-(1-{1-[(2-chloropyridin-3-yl)methyl]piperidin-4-yl}-4-morpholin-4-yl-1H-pyrazolo[3,4-d]pyrimidin-6-yl)phenyl]-3-methylurea;
1-[4-(1-{1-[(4-methoxypyridin-3-yl)methyl]piperidin-4-yl}-4-morpholin-4-yl-1H-pyrazolo[3,4-d]pyrimidin-6-yl)phenyl]-3-methylurea;
1-[4-(1-{1-[(5-fluoropyridin-3-yl)methyl]piperidin-4-yl}-4-morpholin-4-yl-1H-pyrazolo[3,4-d]pyrimidin-6-yl)phenyl]-3-methylurea;
1-[4-(1-{1-[(5-bromopyridin-3-yl)methyl]piperidin-4-yl}-4-morpholin-4-yl-1H-pyrazolo[3,4-d]pyrimidin-6-yl)phenyl]-3-methylurea;
1-(4-{1-[1-(4-chlorobenzyl)piperidin-4-yl]-4-morpholin-4-yl-1H-pyrazolo[3,4-d]pyrimidin-6-yl}phenyl)-3-methylurea;
1-(4-{1-[1-(3-chlorobenzyl)piperidin-4-yl]-4-morpholin-4-yl-1H-pyrazolo[3,4-d]pyrimidin-6-yl}phenyl)-3-methylurea;
1-(4-{1-[1-(2-chlorobenzyl)piperidin-4-yl]-4-morpholin-4-yl-1H-pyrazolo[3,4-d]pyrimidin-6-yl}phenyl)-3-methylurea;
1-(4-{1-[1-(3-hydroxybenzyl)piperidin-4-yl]-4-morpholin-4-yl-1H-pyrazolo[3,4-d]pyrimidin-6-yl}phenyl)-3-methylurea;
1-(4-{1-[1-(3-hydroxy-4-methoxybenzyl)piperidin-4-yl]-4-morpholin-4-yl-1H-pyrazolo[3,4-d]pyrimidin-6-yl}phenyl)-3-methylurea;
1-(4-{1-[1-(2-hydroxybenzyl)piperidin-4-yl]-4-morpholin-4-yl-1H-pyrazolo[3,4-d]pyrimidin-6-yl}phenyl)-3-methylurea;
1-(4-{1-[1-(3-methoxybenzyl)piperidin-4-yl]-4-morpholin-4-yl-1H-pyrazolo[3,4-d]pyrimidin-6-yl}phenyl)-3-methylurea;
1-methyl-3-{4-[1-(1-methylpiperidin-4-yl)-4-morpholin-4-yl-1H-pyrazolo[3,4-d]pyrimidin-6-yl]phenyl}urea;
1-(4-{1-[1-(2-furylmethyl)piperidin-4-yl]-4-morpholin-4-yl-1H-pyrazolo[3,4-d]pyrimidin-6-yl}phenyl)-3-methylurea;
1-(4-{4-morpholin-4-yl-1-[1-(pyridin-3-ylmethyl)piperidin-4-yl]-1H-pyrazolo[3,4-d]pyrimidin-6-yl}phenyl)-3-(2-thienyl)urea;
1-cyclopropyl-3-(4-{4-morpholin-4-yl-1-[1-(pyridin-3-ylmethyl)piperidin-4-yl]-1H-pyrazolo[3,4-d]pyrimidin-6-yl}phenyl)urea;
2-cyano-1-(4-{4-morpholin-4-yl-1-[1-(pyridin-3-ylmethyl)piperidin-4-yl]-1H-pyrazolo[3,4-d]pyrimidin-6-yl}phenyl)guanidine;
2-cyano-1-methyl-3-(4-{4-morpholin-4-yl-1-[1-(pyridin-3-ylmethyl)piperidin-4-yl]-1H-pyrazolo[3,4-d]pyrimidin-6-yl}phenyl)guanidine;
2-cyano-1-ethyl-3-(4-{4-morpholin-4-yl-1-[1-(pyridin-3-ylmethyl)piperidin-4-yl]-1H-pyrazolo[3,4-d]pyrimidin-6-yl}phenyl)guanidine;
N′-cyano-N-(4-{4-morpholin-4-yl-1-[1-(pyridin-3-ylmethyl)piperidin-4-yl]-1H-pyrazolo[3,4-d]pyrimidin-6-yl}phenyl)carbamimidic acid;
methyl N′-cyano-N-(4-{4-morpholin-4-yl-1-[1-(pyridin-3-ylmethyl)piperidin-4-yl]-1H-pyrazolo[3,4-d]pyrimidin-6-yl}phenyl)imidocarbamate;
2-cyano-1-(4-{4-morpholin-4-yl-1-[1-(pyridin-3-ylcarbonyl)piperidin-4-yl]-1H-pyrazolo[3,4-d]pyrimidin-6-yl}phenyl)guanidine;
2-cyano-1-methyl-3-(4-{4-morpholin-4-yl-1-[1-(pyridin-3-ylcarbonyl)piperidin-4-yl]-1H-pyrazolo[3,4-d]pyrimidin-6-yl}phenyl)guanidine;
1-(4-{4-morpholin-4-yl-1-[1-(pyridin-3-ylcarbonyl)piperidin-4-yl]-1H-pyrazolo[3,4-d]pyrimidin-6-yl}phenyl)-3-(2-thienyl)urea;
1-(4-{4-morpholin-4-yl-1-[1-(pyridin-3-ylcarbonyl)piperidin-4-yl]-1H-pyrazolo[3,4-d]pyrimidin-6-yl}phenyl)-3-(3-thienyl)urea;
1-cyclopropyl-3-(4-{4-morpholin-4-yl-1-[1-(pyridin-3-ylcarbonyl)piperidin-4-yl]-1H-pyrazolo[3,4-d]pyrimidin-6-yl}phenyl)urea;
6-(2,3-dihydro-1H-indol-5-yl)-4-morpholin-4-yl-1-[1-(pyridin-3-ylmethyl)piperidin-4-yl]-1H-pyrazolo[3,4-d]pyrimidine;
1-{4-[1-(1-isonicotinoylpiperidin-4-yl)-4-morpholin-4-yl-1H-pyrazolo[3,4-d]pyrimidin-6-yl]phenyl}-3-methylurea;
1-methyl-3-(4-{4-morpholin-4-yl-1-[1-(pyridin-2-ylcarbonyl)piperidin-4-yl]-1H-pyrazolo[3,4-d]pyrimidin-6-yl}phenyl)urea;
1-methyl-3-[4-(1-{1-[(4-methylpyridin-3-yl)carbonyl]piperidin-4-yl}-4-morpholin-4-yl-1H-pyrazolo[3,4-d]pyrimidin-6-yl)phenyl]urea;
1-methyl-3-[4-(1-{1-[(6-methylpyridin-3-yl)carbonyl]piperidin-4-yl}-4-morpholin-4-yl-1H-pyrazolo[3,4-d]pyrimidin-6-yl)phenyl]urea;
1-[4-(1-{1-[(6-fluoropyridin-3-yl)carbonyl]piperidin-4-yl}-4-morpholin-4-yl-1H-pyrazolo[3,4-d]pyrimidin-6-yl)phenyl]-3-methylurea;
1-methyl-3-(4-{4-morpholin-4-yl-1-[1-(pyrazin-2-ylcarbonyl)piperidin-4-yl]-1H-pyrazolo[3,4-d]pyrimidin-6-yl}phenyl)urea;
1-(4-{1-[1-(3-acetylbenzoyl)piperidin-4-yl]-4-morpholin-4-yl-1H-pyrazolo[3,4-d]pyrimidin-6-yl}phenyl)-3-methylurea;
1-[4-(1-{1-[(6-chloropyridin-3-yl)carbonyl]piperidin-4-yl}-4-morpholin-4-yl-1H-pyrazolo[3,4-d]pyrimidin-6-yl)phenyl]-3-methylurea;
1-[4-(1-{1-[(2-chloropyridin-3-yl)carbonyl]piperidin-4-yl}-4-morpholin-4-yl-1H-pyrazolo[3,4-d]pyrimidin-6-yl)phenyl]-3-methylurea;
1-methyl-3-(4-{4-morpholin-4-yl-1-[1-(pyridin-4-ylmethyl)piperidin-4-yl]-1H-pyrazolo[3,4-d]pyrimidin-6-yl}phenyl)urea;
1-(4-{1-[1-(4-fluorobenzyl)piperidin-4-yl]-4-morpholin-4-yl-1H-pyrazolo[3,4-d]pyrimidin-6-yl}phenyl)-3-methylurea;
1-(4-{1-[1-(3-fluorobenzyl)piperidin-4-yl]-4-morpholin-4-yl-1H-pyrazolo[3,4-d]pyrimidin-6-yl}phenyl)-3-methylurea;
1-methyl-3-(4-{1-[1-(2-methylbenzyl)piperidin-4-yl]-4-morpholin-4-yl-1H-pyrazolo[3,4-d]pyrimidin-6-yl}phenyl)urea;
1-methyl-3-(4-{1-[1-(3-methylbenzyl)piperidin-4-yl]-4-morpholin-4-yl-1H-pyrazolo[3,4-d]pyrimidin-6-yl}phenyl)urea;
1-methyl-3-(4-{1-[1-(4-methylbenzyl)piperidin-4-yl]-4-morpholin-4-yl-1H-pyrazolo[3,4-d]pyrimidin-6-yl}phenyl)urea;
6-(1H-indazol-5-yl)-4-morpholin-4-yl-1-phenyl-1H-pyrazolo[3,4-d]pyrimidine;
5-(4-morpholin-4-yl-1-phenyl-1H-pyrazolo[3,4-d]pyrimidin-6-yl)-1,3-dihydro-2H-indol-2-one;
2-{4-morpholin-4-yl-1-[1-(pyridin-3-ylcarbonyl)piperidin-4-yl]-1H-pyrazolo[3,4-d]pyrimidin-6-yl}pyridin-4-amine;
6-{4-morpholin-4-yl-1-[1-(pyridin-3-ylcarbonyl)piperidin-4-yl]-1H-pyrazolo[3,4-d]pyrimidin-6-yl}pyridin-3-amine;
6-{4-morpholin-4-yl-1-[1-(pyridin-3-ylcarbonyl)piperidin-4-yl]-1H-pyrazolo[3,4-d]pyrimidin-6-yl}pyridin-2-amine;
2-(4-morpholin-4-yl-1-phenyl-1H-pyrazolo[3,4-d]pyrimidin-6-yl)pyridin-4-amine;
6-(4-morpholin-4-yl-1-phenyl-1H-pyrazolo[3,4-d]pyrimidin-6-yl)pyridin-3-amine;
6-(4-morpholin-4-yl-1-phenyl-1H-pyrazolo[3,4-d]pyrimidin-6-yl)pyridin-2-amine;
methyl[4-(1-{1-[(4-methylpyridin-3-yl)carbonyl]piperidin-4-yl}-4-morpholin-4-yl-1H-pyrazolo[3,4-d]pyrimidin-6-yl)phenyl]carbamate;
methyl(4-{4-morpholin-4-yl-1-[1-(pyridin-2-ylcarbonyl)piperidin-4-yl]-1H-pyrazolo[3,4-d]pyrimidin-6-yl}phenyl)carbamate;
methyl{4-[1-(1-benzoylpiperidin-4-yl)-4-morpholin-4-yl-1H-pyrazolo[3,4-d]pyrimidin-6-yl]phenyl}carbamate;
methyl(4-{1-[1-(2-fluorobenzoyl)piperidin-4-yl]-4-morpholin-4-yl-1H-pyrazolo[3,4-d]pyrimidin-6-yl}phenyl)carbamate;
methyl(4-{1-[1-(2-chlorobenzoyl)piperidin-4-yl]-4-morpholin-4-yl-1H-pyrazolo[3,4-d]pyrimidin-6-yl}phenyl)carbamate;
methyl(4-{1-[1-(4-fluorobenzoyl)piperidin-4-yl]-4-morpholin-4-yl-1H-pyrazolo[3,4-d]pyrimidin-6-yl}phenyl)carbamate;
methyl(4-{1-[1-(2-methoxybenzoyl)piperidin-4-yl]-4-morpholin-4-yl-1H-pyrazolo[3,4-d]pyrimidin-6-yl}phenyl)carbamate;
methyl(4-{1-[1-(4-cyanobenzoyl)piperidin-4-yl]-4-morpholin-4-yl-1H-pyrazolo[3,4-d]pyrimidin-6-yl}phenyl)carbamate;
methyl[4-(1-{1-[(4-methylpiperazin-1-yl)carbonyl]piperidin-4-yl}-4-morpholin-4-yl-1H-pyrazolo[3,4-d]pyrimidin-6-yl)phenyl]carbamate;
methyl(4-{1-[1-(2,4-difluorobenzoyl)piperidin-4-yl]-4-morpholin-4-yl-1H-pyrazolo[3,4-d]pyrimidin-6-yl}phenyl)carbamate;
methyl(4-{1-[1-(4-fluorobenzyl)piperidin-4-yl]-4-morpholin-4-yl-1H-pyrazolo[3,4-d]pyrimidin-6-yl}phenyl)carbamate;
methyl(4-{1-[1-(4-chlorobenzyl)piperidin-4-yl]-4-morpholin-4-yl-1H-pyrazolo[3,4-d]pyrimidin-6-yl}phenyl)carbamate;
methyl[4-(1-{1-[(2-methoxypyridin-3-yl)methyl]piperidin-4-yl}-4-morpholin-4-yl-1H-pyrazolo[3,4-d]pyrimidin-6-yl)phenyl]carbamate;
methyl[4-(1-{1-[(6-fluoropyridin-3-yl)methyl]piperidin-4-yl}-4-morpholin-4-yl-1H-pyrazolo[3,4-d]pyrimidin-6-yl)phenyl]carbamate;
methyl[4-(1-{1-[(6-chloropyridin-3-yl)methyl]piperidin-4-yl}-4-morpholin-4-yl-1H-pyrazolo[3,4-d]pyrimidin-6-yl)phenyl]carbamate;
methyl(4-{1-[1-(2-chlorobenzyl)piperidin-4-yl]-4-morpholin-4-yl-1H-pyrazolo[3,4-d]pyrimidin-6-yl}phenyl)carbamate;
methyl(4-{1-[1-(2-amino-2-oxoethyl)piperidin-4-yl]-4-morpholin-4-yl-1H-pyrazolo[3,4-d]pyrimidin-6-yl}phenyl)carbamate;
methyl(4-{4-morpholin-4-yl-1-[1-(2-oxo-2-phenylethyl)piperidin-4-yl]-1H-pyrazolo-[3,4-d]pyrimidin-6-yl}phenyl)carbamate;
methyl{4-[1-(1-acryloylpiperidin-4-yl)-4-morpholin-4-yl-1H-pyrazolo[3,4-d]pyrimidin-6-yl]phenyl}carbamate;
methyl[4-(4-morpholin-4-yl-1-piperidin-4-yl-1H-pyrazolo[3,4-d]pyrimidin-6-yl)phenyl]carbamate;
3-fluoro-4-{4-morpholin-4-yl-1-[1-(pyridin-3-ylcarbonyl)piperidin-4-yl]-1H-pyrazolo[3,4-d]pyrimidin-6-yl}aniline;
1-(3-fluoro-4-{4-morpholin-4-yl-1-[1-(pyridin-3-ylcarbonyl)piperidin-4-yl]-1H-pyrazolo[3,4-d]pyrimidin-6-yl}phenyl)-3-methylurea;
1-ethyl-3-(3-fluoro-4-{4-morpholin-4-yl-1-[1-(pyridin-3-ylcarbonyl)piperidin-4-yl]-1H-pyrazolo[3,4-d]pyrimidin-6-yl}phenyl)urea;
1-(2-fluoroethyl)-3-(3-fluoro-4-{4-morpholin-4-yl-1-[1-(pyridin-3-ylcarbonyl)piperidin-4-yl]-1H-pyrazolo[3,4-d]pyrimidin-6-yl}phenyl)urea;
2,5-difluoro-4-{4-morpholin-4-yl-1-[1-(pyridin-3-ylcarbonyl)piperidin-4-yl]-1H-pyrazolo[3,4-d]pyrimidin-6-yl}aniline;
1-(2,5-difluoro-4-{4-morpholin-4-yl-1-[1-(pyridin-3-ylcarbonyl)piperidin-4-yl]-1H-pyrazolo[3,4-d]pyrimidin-6-yl}phenyl)-3-methylurea;
1-(2,5-difluoro-4-{4-morpholin-4-yl-1-[1-(pyridin-3-ylcarbonyl)piperidin-4-yl]-1H-pyrazolo[3,4-d]pyrimidin-6-yl}phenyl)-3-ethylurea;
1-(2,5-difluoro-4-{4-morpholin-4-yl-1-[1-(pyridin-3-ylcarbonyl)piperidin-4-yl]-1H-pyrazolo[3,4-d]pyrimidin-6-yl}phenyl)-3-(2-fluoroethyl)urea;
1-cyclopropyl-3-(2,5-difluoro-4-{4-morpholin-4-yl-1-[1-(pyridin-3-ylcarbonyl)piperidin-4-yl]-1H-pyrazolo[3,4-d]pyrimidin-6-yl}phenyl)urea;
1-(2,5-difluoro-4-{4-morpholin-4-yl-1-[1-(pyridin-3-ylcarbonyl)piperidin-4-yl]-1H-pyrazolo[3,4-d]pyrimidin-6-yl}phenyl)-3-phenylurea;
1-methyl-3-(4-{4-morpholin-4-yl-1-[1-(2,2,2-trifluoroethyl)piperidin-4-yl]-1H-pyrazolo[3,4-d]pyrimidin-6-yl}phenyl)urea;
6-(1H-indol-5-yl)-4-morpholin-4-yl-1-[1-(2,2,2-trifluoroethyl)piperidin-4-yl]-1H-pyrazolo[3,4-d]pyrimidine;
1-{4-[1-(1-acetylpiperidin-4-yl)-4-morpholin-4-yl-1H-pyrazolo[3,4-d]pyrimidin-6-yl]phenyl}-3-methylurea;
N,N-dimethyl-4-(6-{4-[(methylcarbamoyl)amino]phenyl}-4-morpholin-4-yl-1H-pyrazolo[3,4-d]pyrimidin-1-yl)piperidine-1-carboxamide;
1-(4-{1-[1-(methoxyacetyl)piperidin-4-yl]-4-morpholin-4-yl-1H-pyrazolo[3,4-d]pyrimidin-6-yl}phenyl)-3-methylurea;
1-{4-[1-(1-isobutyrylpiperidin-4-yl)-4-morpholin-4-yl-1H-pyrazolo[3,4-d]pyrimidin-6-yl]phenyl}-3-methylurea;
methyl 4-(6-{4-[(methylcarbamoyl)amino]phenyl}-4-morpholin-4-yl-1H-pyrazolo[3,4-d]pyrimidin-1-yl)piperidine-1-carboxylate;
methyl 4-(6-{4-[(methylcarbamoyl)amino]phenyl}-4-morpholin-4-yl-1H-pyrazolo[3,4-d]pyrimidin-1-yl)piperidine-1-carboxylate;
N-methyl-4-(6-{4-[(methylcarbamoyl)amino]phenyl}-4-morpholin-4-yl-1H-pyrazolo[3,4-d]pyrimidin-1-yl)piperidine-1-carboxamide;
3-{4-[(2R,6S)-2,6-dimethylmorpholin-4-yl]-1-phenyl-1H-pyrazolo[3,4-d]pyrimidin-6-yl}phenol;
1-ethyl-3-(5-{4-morpholin-4-yl-1-[1-(pyridin-3-ylmethyl)piperidin-4-yl]-1H-pyrazolo[3,4-d]pyrimidin-6-yl}pyridin-2-yl)urea;
1-methyl-3-(5-{4-morpholin-4-yl-1-[1-(pyridin-3-ylcarbonyl)piperidin-4-yl]-1H-pyrazolo[3,4-d]pyrimidin-6-yl}pyridin-2-yl)urea;
3-{4-[(3S)-3-methylmorpholin-4-yl]-1-phenyl-1H-pyrazolo[3,4-d]pyrimidin-6-yl}phenol;
4-[6-(3-hydroxyphenyl)-1-phenyl-1H-pyrazolo[3,4-d]pyrimidin-4-yl]morpholin-3-one;
3-[4-morpholin-4-yl-1-(2,2,2-trifluoroethyl)-1H-pyrazolo[3,4-d]pyrimidin-6-yl]phenol;
1-methyl-3-{4-[4-morpholin-4-yl-1-(2,2,2-trifluoroethyl)-1H-pyrazolo[3,4-d]pyrimidin-6-yl]phenyl}urea;
1-(2-chloro-4-{4-morpholin-4-yl-1-[1-(pyridin-3-ylmethyl)piperidin-4-yl]-1H-pyrazolo[3,4-d]pyrimidin-6-yl}phenyl)-3-methylurea;
methyl 4-(6-{4-[(ethylcarbamoyl)amino]phenyl}-4-morpholin-4-yl-1H-pyrazolo[3,4-d]pyrimidin-1-yl)piperidine-1-carboxylate;
methyl 4-[6-(4-{[(2-fluoroethyl)carbamoyl]amino}phenyl)-4-morpholin-4-yl-1H-pyrazolo[3,4-d]pyrimidin-1-yl]piperidine-1-carboxylate;
methyl 4-[6-(4-{[(2-hydroxyethyl)carbamoyl]amino}phenyl)-4-morpholin-4-yl-1H-pyrazolo[3,4-d]pyrimidin-1-yl]piperidine-1-carboxylate;
methyl 4-(6-{4-[(cyclopropylcarbamoyl)amino]phenyl}-4-morpholin-4-yl-1H-pyrazolo[3,4-d]pyrimidin-1-yl)piperidine-1-carboxylate;
methyl 4-(6-{4-[(anilinocarbonyl)amino]phenyl}-4-morpholin-4-yl-1H-pyrazolo[3,4-d]pyrimidin-1-yl)piperidine-1-carboxylate;
methyl 4-(4-morpholin-4-yl-6-{4-[(pyridin-2-ylcarbamoyl)amino]phenyl}-1H-pyrazolo[3,4-d]pyrimidin-1-yl)piperidine-1-carboxylate;
methyl 4-(4-morpholin-4-yl-6-{4-[(pyridin-3-ylcarbamoyl)amino]phenyl}-1H-pyrazolo[3,4-d]pyrimidin-1-yl)piperidine-1-carboxylate;
methyl 4-(4-morpholin-4-yl-6-{4-[(pyridin-4-ylcarbamoyl)amino]phenyl}-1H-pyrazolo[3,4-d]pyrimidin-1-yl)piperidine-1-carboxylate;
methyl 4-(6-{4-[(methoxycarbonyl)amino]phenyl}-4-morpholin-4-yl-1H-pyrazolo[3,4-d]pyrimidin-1-yl)piperidine-1-carboxylate;
methyl 4-(6-{4-[(methoxycarbonyl)amino]phenyl}-4-morpholin-4-yl-1H-pyrazolo[3,4-d]pyrimidin-1-yl)piperidine-1-carboxylate;
methyl 4-[6-(4-aminophenyl)-4-morpholin-4-yl-1H-pyrazolo[3,4-d]pyrimidin-1-yl]piperidine-1-carboxylate;
methyl 4-[6-(4-{[(methylamino)carbonothioyl]amino}phenyl)-4-morpholin-4-yl-1H-pyrazolo[3,4-d]pyrimidin-1-yl]piperidine-1-carboxylate;
1-(4-{1-[1-(4-hydroxybutyl)piperidin-4-yl]-4-morpholin-4-yl-1H-pyrazolo[3,4-d]pyrimidin-6-yl}phenyl)-3-methylurea;
methyl (4-{1-[1-(4-hydroxybutyl)piperidin-4-yl]-4-morpholin-4-yl-1H-pyrazolo[3,4-d]pyrimidin-6-yl}phenyl)carbamate;
1-ethyl-3-(4-{1-[1-(4-hydroxybutyl)piperidin-4-yl]-4-morpholin-4-yl-1H-pyrazolo[3,4-d]pyrimidin-6-yl}phenyl)urea;
1-(4-{1-[1-(4-hydroxybutyl)piperidin-4-yl]-4-morpholin-4-yl-1H-pyrazolo[3,4-d]pyrimidin-6-yl}phenyl)-3-(2-hydroxyethyl)urea;
1-(2-fluoroethyl)-3-(4-{1-[1-(4-hydroxybutyl)piperidin-4-yl]-4-morpholin-4-yl-1H-pyrazolo[3,4-d]pyrimidin-6-yl}phenyl)urea;
1-(4-{1-[1-(4-hydroxybutyl)piperidin-4-yl]-4-morpholin-4-yl-1H-pyrazolo[3,4-d]pyrimidin-6-yl}phenyl)-3-phenylurea;
4-{4-[6-(4-aminophenyl)-4-morpholin-4-yl-1H-pyrazolo[3,4-d]pyrimidin-1-yl]piperidin-1-yl}butan-1-ol;
ethyl 4-(6-{4-[(methylcarbamoyl)amino]phenyl}-4-morpholin-4-yl-1H-pyrazolo[3,4-d]pyrimidin-1-yl)piperidine-1-carboxylate;
propyl 4-(6-{4-[(methylcarbamoyl)amino]phenyl}-4-morpholin-4-yl-1H-pyrazolo[3,4-d]pyrimidin-1-yl)piperidine-1-carboxylate;
isopropyl 4-(6-{4-[(methylcarbamoyl)amino]phenyl}-4-morpholin-4-yl-1H-pyrazolo[3,4-d]pyrimidin-1-yl)piperidine-1-carboxylate;
vinyl 4-(6-{4-[(methylcarbamoyl)amino]phenyl}-4-morpholin-4-yl-1H-pyrazolo[3,4-d]pyrimidin-1-yl)piperidine-1-carboxylate;
isobutyl 4-(6-{4-[(methylcarbamoyl)amino]phenyl}-4-morpholin-4-yl-1H-pyrazolo[3,4-d]pyrimidin-1-yl)piperidine-1-carboxylate;
phenyl 4-(6-{4-[(methylcarbamoyl)amino]phenyl}-4-morpholin-4-yl-1H-pyrazolo[3,4-d]pyrimidin-1-yl)piperidine-1-carboxylate;
1-[4-(1-{1-[(2E)-but-2-enoyl]piperidin-4-yl}-4-morpholin-4-yl-1H-pyrazolo[3,4-d]pyrimidin-6-yl)phenyl]-3-methylurea;
methyl 3-[4-(6-{4-[(methylcarbamoyl)amino]phenyl}-4-morpholin-4-yl-1H-pyrazolo[3,4-d]pyrimidin-1-yl)piperidin-1-yl]-3-oxopropanoate;
1-{4-[1-(1-acryloylpiperidin-4-yl)-4-morpholin-4-yl-1H-pyrazolo[3,4-d]pyrimidin-6-yl]phenyl}-3-methylurea;
1-methyl-3-(4-{1-[1-(methylsulfonyl)piperidin-4-yl]-4-morpholin-4-yl-1H-pyrazolo[3,4-d]pyrimidin-6-yl}phenyl)urea;
N-methyl-4-(6-{4-[(methylcarbamoyl)amino]phenyl}-4-morpholin-4-yl-1H-pyrazolo[3,4-d]pyrimidin-1-yl)piperidine-1-carbothioamide;
S-methyl 4-(6-{4-[(methylcarbamoyl)amino]phenyl}-4-morpholin-4-yl-1H-pyrazolo[3,4-d]pyrimidin-1-yl)piperidine-1-carbothioate;
S-ethyl 4-(6-{4-[(methylcarbamoyl)amino]phenyl}-4-morpholin-4-yl-1H-pyrazolo[3,4-d]pyrimidin-1-yl)piperidine-1-carbothioate;
1-methyl-3-(4-{4-morpholin-4-yl-1-[1-(trifluoroacetyl)piperidin-4-yl]-1H-pyrazolo[3,4-d]pyrimidin-6-yl}phenyl)urea;
tert-butyl 4-(6-{4-[(ethylcarbamoyl)amino]phenyl}-4-morpholin-4-yl-1H-pyrazolo[3,4-d]pyrimidin-1-yl)piperidine-1-carboxylate;
tert-butyl 4-[6-(4-{[(2-fluoroethyl)carbamoyl]amino}phenyl)-4-morpholin-4-yl-1H-pyrazolo[3,4-d]pyrimidin-1-yl]piperidine-1-carboxylate;
tert-butyl 4-[6-(4-{[(2-hydroxyethyl)carbamoyl]amino}phenyl)-4-morpholin-4-yl-1H-pyrazolo[3,4-d]pyrimidin-1-yl]piperidine-1-carboxylate;
tert-butyl 4-(6-{4-[(cyclopropylcarbamoyl)amino]phenyl}-4-morpholin-4-yl-1H-pyrazolo[3,4-d]pyrimidin-1-yl)piperidine-1-carboxylate;
tert-butyl 4-(6-{4-[(anilinocarbonyl)amino]phenyl}-4-morpholin-4-yl-1H-pyrazolo[3,4-d]pyrimidin-1-yl)piperidine-1-carboxylate;
tert-butyl 4-(4-morpholin-4-yl-6-{4-[(pyridin-2-ylcarbamoyl)amino]phenyl}-1H-pyrazolo[3,4-d]pyrimidin-1-yl)piperidine-1-carboxylate;
tert-butyl 4-(4-morpholin-4-yl-6-{4-[(pyridin-3-ylcarbamoyl)amino]phenyl}-1H-pyrazolo[3,4-d]pyrimidin-1-yl)piperidine-1-carboxylate;
tert-butyl 4-(4-morpholin-4-yl-6-{4-[(pyridin-4-ylcarbamoyl)amino]phenyl}-1H-pyrazolo[3,4-d]pyrimidin-1-yl)piperidine-1-carboxylate;
tert-butyl 4-(6-{4-[(methoxycarbonyl)amino]phenyl}-4-morpholin-4-yl-1H-pyrazolo[3,4-d]pyrimidin-1-yl)piperidine-1-carboxylate;
1-ethyl-3-[4-(4-morpholin-4-yl-1-piperidin-4-yl-1H-pyrazolo[3,4-d]pyrimidin-6-yl)phenyl]urea;
1-(2-fluoroethyl)-3-[4-(4-morpholin-4-yl-1-piperidin-4-yl-1H-pyrazolo[3,4-d]pyrimidin-6-yl)phenyl]urea;
1-(2-hydroxyethyl)-3-[4-(4-morpholin-4-yl-1-piperidin-4-yl-1H-pyrazolo[3,4-d]pyrimidin-6-yl)phenyl]urea;
1-cyclopropyl-3-[4-(4-morpholin-4-yl-1-piperidin-4-yl-1H-pyrazolo[3,4-d]pyrimidin-6-yl)phenyl]urea;
1-[4-(4-morpholin-4-yl-1-piperidin-4-yl-1H-pyrazolo[3,4-d]pyrimidin-6-yl)phenyl]-3-phenylurea;
1-[4-(4-morpholin-4-yl-1-piperidin-4-yl-1H-pyrazolo[3,4-d]pyrimidin-6-yl)phenyl]-3-pyridin-2-ylurea;
1-[4-(4-morpholin-4-yl-1-piperidin-4-yl-1H-pyrazolo[3,4-d]pyrimidin-6-yl)phenyl]-3-pyridin-3-ylurea;
1-[4-(4-morpholin-4-yl-1-piperidin-4-yl-1H-pyrazolo[3,4-d]pyrimidin-6-yl)phenyl]-3-pyridin-4-ylurea;
methyl(4-{4-morpholin-4-yl-1-[1-(2,2,2-trifluoroethyl)piperidin-4-yl]-1H-pyrazolo[3,4-d]pyrimidin-6-yl}phenyl)carbamate;
1-ethyl-3-(4-{4-morpholin-4-yl-1-[1-(2,2,2-trifluoroethyl)piperidin-4-yl]-1H-pyrazolo[3,4-d]pyrimidin-6-yl}phenyl)urea;
1-(2-fluoroethyl)-3-(4-{4-morpholin-4-yl-1-[1-(2,2,2-trifluoroethyl)piperidin-4-yl]-1H-pyrazolo[3,4-d]pyrimidin-6-yl}phenyl)urea;
1-(2-hydroxyethyl)-3-(4-{4-morpholin-4-yl-1-[1-(2,2,2-trifluoroethyl)piperidin-4-yl]-1H-pyrazolo[3,4-d]pyrimidin-6-yl}phenyl)urea;
1-(4-{4-morpholin-4-yl-1-[1-(2,2,2-trifluoroethyl)piperidin-4-yl]-1H-pyrazolo[3,4-d]pyrimidin-6-yl}phenyl)-3-pyridin-2-ylurea;
1-(4-{4-morpholin-4-yl-1-[1-(2,2,2-trifluoroethyl)piperidin-4-yl]-1H-pyrazolo[3,4-d]pyrimidin-6-yl}phenyl)-3-pyridin-4-ylurea;
1-(4-{4-morpholin-4-yl-1-[1-(2,2,2-trifluoroethyl)piperidin-4-yl]-1H-pyrazolo[3,4-d]pyrimidin-6-yl}phenyl)-3-pyridin-3-ylurea;
1-cyclopropyl-3-(4-{4-morpholin-4-yl-1-[1-(2,2,2-trifluoroethyl)piperidin-4-yl]-1H-pyrazolo[3,4-d]pyrimidin-6-yl}phenyl)urea;
1-(4-{4-morpholin-4-yl-1-[1-(2,2,2-trifluoroethyl)piperidin-4-yl]-1H-pyrazolo[3,4-d]pyrimidin-6-yl}phenyl)-3-phenylurea;
1-(2-fluoroethyl)-3-{4-[4-morpholin-4-yl-1-(2,2,2-trifluoroethyl)-1H-pyrazolo[3,4-d]pyrimidin-6-yl]phenyl}urea;
1-(2-hydroxyethyl)-3-{4-[4-morpholin-4-yl-1-(2,2,2-trifluoroethyl)-1H-pyrazolo[3,4-d]pyrimidin-6-yl]phenyl}urea;
1-{4-[4-morpholin-4-yl-1-(2,2,2-trifluoroethyl)-1H-pyrazolo[3,4-d]pyrimidin-6-yl]phenyl}-3-pyridin-3-ylurea;
1-(4-{1-[1-(cyanomethyl)piperidin-4-yl]-4-morpholin-4-yl-1H-pyrazolo[3,4-d]pyrimidin-6-yl}phenyl)-3-methylurea;
methyl[4-(6-{4-[(methylcarbamoyl)amino]phenyl}-4-morpholin-4-yl-1H-pyrazolo[3,4-d]pyrimidin-1-yl)piperidin-1-yl]acetate;
ethyl[4-(6-{4-[(methylcarbamoyl)amino]phenyl}-4-morpholin-4-yl-1H-pyrazolo[3,4-d]pyrimidin-1-yl)piperidin-1-yl]acetate;
1-(4-{1-[1-(methoxymethyl)piperidin-4-yl]-4-morpholin-4-yl-1H-pyrazolo[3,4-d]pyrimidin-6-yl}phenyl)-3-methylurea;
1-(4-{1-[1-(1,3-dioxolan-2-ylmethyl)piperidin-4-yl]-4-morpholin-4-yl-1H-pyrazolo[3,4-d]pyrimidin-6-yl}phenyl)-3-methylurea;
[4-(6-{4-[(methylcarbamoyl)amino]phenyl}-4-morpholin-4-yl-1H-pyrazolo[3,4-d]pyrimidin-1-yl)piperidin-1-yl]acetic acid;
1-{4-[1-(1-allylpiperidin-4-yl)-4-morpholin-4-yl-1H-pyrazolo[3,4-d]pyrimidin-6-yl]phenyl}-3-methylurea;
2-methoxyethyl 4-(6-{4-[(methylcarbamoyl)amino]phenyl}-4-morpholin-4-yl-1H-pyrazolo[3,4-d]pyrimidin-1-yl)piperidine-1-carboxylate;
but-2-yn-1-yl 4-(6-{4-[(methylcarbamoyl)amino]phenyl}-4-morpholin-4-yl-1H-pyrazolo[3,4-d]pyrimidin-1-yl)piperidine-1-carboxylate;
2-(methylamino)ethyl 4-(6-{4-[(methylcarbamoyl)amino]phenyl}-4-morpholin-4-yl-1H-pyrazolo[3,4-d]pyrimidin-1-yl)piperidine-1-carboxylate;
2-(dimethylamino)ethyl 4-(6-{4-[(methylcarbamoyl)amino]phenyl}-4-morpholin-4-yl-1H-pyrazolo[3,4-d]pyrimidin-1-yl)piperidine-1-carboxylate;
2-bromoethyl 4-(6-{4-[(methylcarbamoyl)amino]phenyl}-4-morpholin-4-yl-1H-pyrazolo[3,4-d]pyrimidin-1-yl)piperidine-1-carboxylate;
ethyl 4-(6-{4-[(methoxycarbonyl)amino]phenyl}-4-morpholin-4-yl-1H-pyrazolo[3,4-d]pyrimidin-1-yl)piperidine-1-carboxylate;
isopropyl 4-(6-{4-[(methoxycarbonyl)amino]phenyl}-4-morpholin-4-yl-1H-pyrazolo[3,4-d]pyrimidin-1-yl)piperidine-1-carboxylate;
S-ethyl 4-(6-{4-[(methoxycarbonyl)amino]phenyl}-4-morpholin-4-yl-1H-pyrazolo[3,4-d]pyrimidin-1-yl)piperidine-1-carbothioate;
methyl(4-{1-[1-(dimethylcarbamoyl)piperidin-4-yl]-4-morpholin-4-yl-1H-pyrazolo[3,4-d]pyrimidin-6-yl}phenyl)carbamate;
methyl{4-[1-(1-acetylpiperidin-4-yl)-4-morpholin-4-yl-1H-pyrazolo[3,4-d]pyrimidin-6-yl]phenyl}carbamate;
methyl{4-[1-(1-isobutyrylpiperidin-4-yl)-4-morpholin-4-yl-1H-pyrazolo[3,4-d]pyrimidin-6-yl]phenyl}carbamate;
methyl(4-{4-morpholin-4-yl-1-[1-(trifluoroacetyl)piperidin-4-yl]-1H-pyrazolo[3,4-d]pyrimidin-6-yl}phenyl)carbamate;
methyl[4-(1-{1-[(ethylamino)carbonothioyl]piperidin-4-yl}-4-morpholin-4-yl-1H-pyrazolo[3,4-d]pyrimidin-6-yl)phenyl]carbamate;
methyl(4-{1-[1-(methylcarbamoyl)piperidin-4-yl]-4-morpholin-4-yl-1H-pyrazolo[3,4-d]pyrimidin-6-yl}phenyl)carbamate;
ethyl 4-(6-{4-[(anilinocarbonyl)amino]phenyl}-4-morpholin-4-yl-1H-pyrazolo[3,4-d]pyrimidin-1-yl)piperidine-1-carboxylate;
ethyl 4-(4-morpholin-4-yl-6-{4-[(pyridin-2-ylcarbamoyl)amino]phenyl}-1H-pyrazolo[3,4-d]pyrimidin-1-yl)piperidine-1-carboxylate;
ethyl 4-(4-morpholin-4-yl-6-{4-[(pyridin-3-ylcarbamoyl)amino]phenyl}-1H-pyrazolo[3,4-d]pyrimidin-1-yl)piperidine-1-carboxylate;
ethyl 4-(4-morpholin-4-yl-6-{4-[(pyridin-4-ylcarbamoyl)amino]phenyl}-1H-pyrazolo[3,4-d]pyrimidin-1-yl)piperidine-1-carboxylate;
ethyl 4-[6-(4-{[(2-hydroxyethyl)carbamoyl]amino}phenyl)-4-morpholin-4-yl-1H-pyrazolo[3,4-d]pyrimidin-1-yl]piperidine-1-carboxylate;
ethyl 4-[6-(4-{[(2-fluoroethyl)carbamoyl]amino}phenyl)-4-morpholin-4-yl-1H-pyrazolo[3,4-d]pyrimidin-1-yl]piperidine-1-carboxylate;
ethyl 4-(6-{4-[(ethylcarbamoyl)amino]phenyl}-4-morpholin-4-yl-1H-pyrazolo[3,4-d]pyrimidin-1-yl)piperidine-1-carboxylate;
ethyl 4-(6-{4-[(cyclopropylcarbamoyl)amino]phenyl}-4-morpholin-4-yl-1H-pyrazolo[3,4-d]pyrimidin-1-yl)piperidine-1-carboxylate;
1-ethyl-3-{4-[1-(1-isobutyrylpiperidin-4-yl)-4-morpholin-4-yl-1H-pyrazolo[3,4-d]pyrimidin-6-yl]phenyl}urea;
1-(2-hydroxyethyl)-3-{4-[1-(1-isobutyrylpiperidin-4-yl)-4-morpholin-4-yl-1H-pyrazolo[3,4-d]pyrimidin-6-yl]phenyl}urea;
1-cyclopropyl-3-{4-[1-(1-isobutyrylpiperidin-4-yl)-4-morpholin-4-yl-1H-pyrazolo[3,4-d]pyrimidin-6-yl]phenyl}urea;
1-(2-fluoroethyl)-3-{4-[1-(1-isobutyrylpiperidin-4-yl)-4-morpholin-4-yl-1H-pyrazolo[3,4-d]pyrimidin-6-yl]phenyl}urea;
1-{4-[1-(1-isobutyrylpiperidin-4-yl)-4-morpholin-4-yl-1H-pyrazolo[3,4-d]pyrimidin-6-yl]phenyl}-3-phenylurea;
1-{4-[1-(1-isobutyrylpiperidin-4-yl)-4-morpholin-4-yl-1H-pyrazolo[3,4-d]pyrimidin-6-yl]phenyl}-3-pyridin-2-ylurea;
1-{4-[1-(1-isobutyrylpiperidin-4-yl)-4-morpholin-4-yl-1H-pyrazolo[3,4-d]pyrimidin-6-yl]phenyl}-3-pyridin-3-ylurea;
1-{4-[1-(1-isobutyrylpiperidin-4-yl)-4-morpholin-4-yl-1H-pyrazolo[3,4-d]pyrimidin-6-yl]phenyl}-3-pyridin-4-ylurea;
isopropyl 4-[6-(4-aminophenyl)-4-morpholin-4-yl-1H-pyrazolo[3,4-d]pyrimidin-1-yl]piperidine-1-carboxylate;
isopropyl 4-[6-(4-{[(methylamino)carbonothioyl]amino}phenyl)-4-morpholin-4-yl-1H-pyrazolo[3,4-d]pyrimidin-1-yl]piperidine-1-carboxylate;
isopropyl 4-[6-(4-{[(1E)-(methylamino)(methylthio)methylene]amino}phenyl)-4-morpholin-4-yl-1H-pyrazolo[3,4-d]pyrimidin-1-yl]piperidine-1-carboxylate;
isopropyl 4-[6-(4-{[(2-fluoroethyl)carbamoyl]amino}phenyl)-4-morpholin-4-yl-1H-pyrazolo[3,4-d]pyrimidin-1-yl]piperidine-1-carboxylate;
isopropyl 4-[6-(4-{[(2-hydroxyethyl)carbamoyl]amino}phenyl)-4-morpholin-4-yl-1H-pyrazolo[3,4-d]pyrimidin-1-yl]piperidine-1-carboxylate;
isopropyl 4-(6-{4-[(cyclopropylcarbamoyl)amino]phenyl}-4-morpholin-4-yl-1H-pyrazolo[3,4-d]pyrimidin-1-yl)piperidine-1-carboxylate;
isopropyl 4-(6-{4-[(anilinocarbonyl)amino]phenyl}-4-morpholin-4-yl-1H-pyrazolo[3,4-d]pyrimidin-1-yl)piperidine-1-carboxylate;
isopropyl 4-(4-morpholin-4-yl-6-{4-[(pyridin-2-ylcarbamoyl)amino]phenyl}-1H-pyrazolo[3,4-d]pyrimidin-1-yl)piperidine-1-carboxylate;
isopropyl 4-(4-morpholin-4-yl-6-{4-[(pyridin-3-ylcarbamoyl)amino]phenyl}-1H-pyrazolo[3,4-d]pyrimidin-1-yl)piperidine-1-carboxylate;
isopropyl 4-(4-morpholin-4-yl-6-{4-[(pyridin-4-ylcarbamoyl)amino]phenyl}-1H-pyrazolo[3,4-d]pyrimidin-1-yl)piperidine-1-carboxylate;
methyl 4-[6-{4-[(methoxycarbonyl)amino]phenyl}-4-(2-methylmorpholin-4-yl)-1H-pyrazolo[3,4-d]pyrimidin-1-yl]piperidine-1-carboxylate;
methyl 4-[6-{4-[(methylcarbamoyl)amino]phenyl}-4-(2-methylmorpholin-4-yl)-1H-pyrazolo[3,4-d]pyrimidin-1-yl]piperidine-1-carboxylate;
methyl 4-[6-{4-[(ethylcarbamoyl)amino]phenyl}-4-(2-methylmorpholin-4-yl)-1H-pyrazolo[3,4-d]pyrimidin-1-yl]piperidine-1-carboxylate;
methyl 4-[6-{4-[(cyclopropylcarbamoyl)amino]phenyl}-4-(2-methylmorpholin-4-yl)-1H-pyrazolo[3,4-d]pyrimidin-1-yl]piperidine-1-carboxylate;
methyl 4-[6-(4-{[(2-fluoroethyl)carbamoyl]amino}phenyl)-4-(2-methylmorpholin-4-yl)-1H-pyrazolo[3,4-d]pyrimidin-1-yl]piperidine-1-carboxylate;
methyl 4-[6-(4-{[(2-hydroxyethyl)carbamoyl]amino}phenyl)-4-(2-methylmorpholin-4-yl)-1H-pyrazolo[3,4-d]pyrimidin-1-yl]piperidine-1-carboxylate;
methyl 4-[4-(2-methylmorpholin-4-yl)-6-{4-[(pyridin-3-ylcarbamoyl)amino]phenyl}-1H-pyrazolo[3,4-d]pyrimidin-1-yl]piperidine-1-carboxylate;
methyl 4-[4-(2-methylmorpholin-4-yl)-6-{4-[(pyridin-2-ylcarbamoyl)amino]phenyl}-1H-pyrazolo[3,4-d]pyrimidin-1-yl]piperidine-1-carboxylate;
methyl 4-[6-{4-[(anilinocarbonyl)amino]phenyl}-4-(2-methylmorpholin-4-yl)-1H-pyrazolo[3,4-d]pyrimidin-1-yl]piperidine-1-carboxylate;
propyl 4-(4-morpholin-4-yl-6-{4-[(phenylcarbamoyl)amino]phenyl}-1H-pyrazolo[3,4-d]pyrimidin-1-yl)piperidine-1-carboxylate;
propyl 4-(4-morpholin-4-yl-6-{4-[(pyridin-3-ylcarbamoyl)amino]phenyl}-1H-pyrazolo[3,4-d]pyrimidin-1-yl)piperidine-1-carboxylate;
propyl 4-(4-morpholin-4-yl-6-{4-[(pyridin-4-ylcarbamoyl)amino]phenyl}-1H-pyrazolo[3,4-d]pyrimidin-1-yl)piperidine-1-carboxylate;
propyl 4-(6-{4-[(ethylcarbamoyl)amino]phenyl}-4-morpholin-4-yl-1H-pyrazolo[3,4-d]pyrimidin-1-yl)piperidine-1-carboxylate;
propyl 4-(4-morpholin-4-yl-6-{4-[(propylcarbamoyl)amino]phenyl}-1H-pyrazolo[3,4-d]pyrimidin-1-yl)piperidine-1-carboxylate;
propyl 4-[6-(4-{[(2-fluoroethyl)carbamoyl]amino}phenyl)-4-morpholin-4-yl-1H-pyrazolo[3,4-d]pyrimidin-1-yl]piperidine-1-carboxylate;
propyl 4-[6-(4-{[(2-hydroxyethyl)carbamoyl]amino}phenyl)-4-morpholin-4-yl-1H-pyrazolo[3,4-d]pyrimidin-1-yl]piperidine-1-carboxylate;
propyl 4-(6-{4-[(cyclopropylcarbamoyl)amino]phenyl}-4-morpholin-4-yl-1H-pyrazolo[3,4-d]pyrimidin-1-yl)piperidine-1-carboxylate;
propyl 4-(6-{4-[(methoxycarbonyl)amino]phenyl}-4-morpholin-4-yl-1H-pyrazolo[3,4-d]pyrimidin-1-yl)piperidine-1-carboxylate;
propyl 4-[6-(4-{[(cyclopropylmethyl)carbamoyl]amino}phenyl)-4-morpholin-4-yl-1H-pyrazolo[3,4-d]pyrimidin-1-yl]piperidine-1-carboxylate;
methyl 4-[6-(4-{[(4-fluorophenyl)carbamoyl]amino}phenyl)-4-morpholin-4-yl-1H-pyrazolo[3,4-d]pyrimidin-1-yl]piperidine-1-carboxylate;
methyl 4-[6-(4-{[(2-fluorophenyl)carbamoyl]amino}phenyl)-4-morpholin-4-yl-1H-pyrazolo[3,4-d]pyrimidin-1-yl]piperidine-1-carboxylate;
methyl 4-[6-(4-{[(2,4-difluorophenyl)carbamoyl]amino}phenyl)-4-morpholin-4-yl-1H-pyrazolo[3,4-d]pyrimidin-1-yl]piperidine-1-carboxylate;
methyl 4-[6-(4-{[(6-fluoropyridin-3-yl)carbamoyl]amino}phenyl)-4-morpholin-4-yl-1H-pyrazolo[3,4-d]pyrimidin-1-yl]piperidine-1-carboxylate;
methyl 4-[6-(4-{[(2-fluoropyridin-3-yl)carbamoyl]amino}phenyl)-4-morpholin-4-yl-1H-pyrazolo[3,4-d]pyrimidin-1-yl]piperidine-1-carboxylate;
methyl 4-[6-(4-{[(3-fluoropyridin-4-yl)carbamoyl]amino}phenyl)-4-morpholin-4-yl-1H-pyrazolo[3,4-d]pyrimidin-1-yl]piperidine-1-carboxylate;
methyl 4-[6-(4-{[(2-fluoroethoxy)carbonyl]amino}phenyl)-4-morpholin-4-yl-1H-pyrazolo[3,4-d]pyrimidin-1-yl]piperidine-1-carboxylate;
methyl 4-[6-(4-{[(2-fluorophenoxy)carbonyl]amino}phenyl)-4-morpholin-4-yl-1H-pyrazolo[3,4-d]pyrimidin-1-yl]piperidine-1-carboxylate;
1-methyl-3-{4-[4-morpholin-4-yl-1-(tetrahydro-2H-thiopyran-4-yl)-1H-pyrazolo[3,4-d]pyrimidin-6-yl]phenyl}urea;
1-methyl-3-{4-[4-morpholin-4-yl-1-(1-oxidotetrahydro-2H-thiopyran-4-yl)-1H-pyrazolo[3,4-d]pyrimidin-6-yl]phenyl}urea;
1-{4-[1-(1,1-dioxidotetrahydro-2H-thiopyran-4-yl)-4-morpholin-4-yl-1H-pyrazolo[3,4-d]pyrimidin-6-yl]phenyl}-3-methylurea;
tert-butyl(3S)-3-[6-(4-aminophenyl)-4-morpholin-4-yl-1H-pyrazolo[3,4-d]pyrimidin-1-yl]piperidine-1-carboxylate;
tert-butyl(3R)-3-[6-(4-aminophenyl)-4-morpholin-4-yl-1H-pyrazolo[3,4-d]pyrimidin-1-yl]piperidine-1-carboxylate;
tert-butyl(3S)-3-(6-{4-[(methylcarbamoyl)amino]phenyl}-4-morpholin-4-yl-1H-pyrazolo[3,4-d]pyrimidin-1-yl)piperidine-1-carboxylate;
1-methyl-3-(4-{4-morpholin-4-yl-1-[(3S)-piperidin-3-yl]-1H-pyrazolo[3,4-d]pyrimidin-6-yl}phenyl)urea;
tert-butyl(3R)-3-(6-{4-[(methylcarbamoyl)amino]phenyl}-4-morpholin-4-yl-1H-pyrazolo[3,4-d]pyrimidin-1-yl)piperidine-1-carboxylate;
1-methyl-3-(4-{4-morpholin-4-yl-1-[(3R)-piperidin-3-yl]-1H-pyrazolo[3,4-d]pyrimidin-6-yl}phenyl)urea;
2,2-dimethylpropyl 4-(6-{4-[(methylcarbamoyl)amino]phenyl}-4-morpholin-4-yl-1H-pyrazolo[3,4-d]pyrimidin-1-yl)piperidine-1-carboxylate;
2,2-dimethylpropyl 4-(4-morpholin-4-yl-6-{4-[(pyridin-3-ylcarbamoyl)amino]phenyl}-1H-pyrazolo[3,4-d]pyrimidin-1-yl)piperidine-1-carboxylate;
2-fluoroethyl 4-(6-{4-[(methylcarbamoyl)amino]phenyl}-4-morpholin-4-yl-1H-pyrazolo[3,4-d]pyrimidin-1-yl)piperidine-1-carboxylate;
2-fluoroethyl 4-(4-morpholin-4-yl-6-{4-[(pyridin-3-ylcarbamoyl)amino]phenyl}-1H-pyrazolo[3,4-d]pyrimidin-1-yl)piperidine-1-carboxylate;
benzyl 4-(6-{4-[(methylcarbamoyl)amino]phenyl}-4-morpholin-4-yl-1H-pyrazolo[3,4-d]pyrimidin-1-yl)piperidine-1-carboxylate;
benzyl 4-(4-morpholin-4-yl-6-{4-[(pyridin-3-ylcarbamoyl)amino]phenyl}-1H-pyrazolo[3,4-d]pyrimidin-1-yl)piperidine-1-carboxylate;
tert-butyl 4-(6-{4-[(isoxazol-3-ylcarbamoyl)amino]phenyl}-4-morpholin-4-yl-1H-pyrazolo[3,4-d]pyrimidin-1-yl)piperidine-1-carboxylate;
tert-butyl 4-[6-(4-{[(3-methylisoxazol-5-yl)carbamoyl]amino}phenyl)-4-morpholin-4-yl-1H-pyrazolo[3,4-d]pyrimidin-1-yl]piperidine-1-carboxylate;
tert-butyl 4-(4-morpholin-4-yl-6-{4-[(1,3-thiazol-2-ylcarbamoyl)amino]phenyl}-1H-pyrazolo[3,4-d]pyrimidin-1-yl)piperidine-1-carboxylate;
tert-butyl 4-(4-morpholin-4-yl-6-{4-[(pyrazin-2-ylcarbamoyl)amino]phenyl}-1H-pyrazolo[3,4-d]pyrimidin-1-yl)piperidine-1-carboxylate;
tert-butyl 4-(4-morpholin-4-yl-6-{4-[(pyrimidin-2-ylcarbamoyl)amino]phenyl}-1H-pyrazolo[3,4-d]pyrimidin-1-yl)piperidine-1-carboxylate;
ethyl 4-{6-[4-(1H-imidazol-2-ylamino)phenyl]-4-morpholin-4-yl-1H-pyrazolo[3,4-d]pyrimidin-1-yl}piperidine-1-carboxylate;
ethyl 4-(6-{4-[(methylcarbamoyl)amino]phenyl}-4-[(3R)-3-methylmorpholin-4-yl]-1H-pyrazolo[3,4-d]pyrimidin-1-yl)piperidine-1-carboxylate;
ethyl 4-(6-{4-[(ethylcarbamoyl)amino]phenyl}-4-[(3R)-3-methylmorpholin-4-yl]-1H-pyrazolo[3,4-d]pyrimidin-1-yl)piperidine-1-carboxylate;
ethyl 4-{6-(4-{[(2-fluoroethyl)carbamoyl]amino}phenyl)-4-[(3R)-3-methylmorpholin-4-yl]-1H-pyrazolo[3,4-d]pyrimidin-1-yl}piperidine-1-carboxylate;
ethyl 4-(4-[(3R)-3-methylmorpholin-4-yl]-6-{4-[(phenylcarbamoyl)amino]phenyl}-1H-pyrazolo[3,4-d]pyrimidin-1-yl)piperidine-1-carboxylate;
ethyl 4-(4-[(3R)-3-methylmorpholin-4-yl]-6-{4-[(pyridin-3-ylcarbamoyl)amino]phenyl}-1H-pyrazolo[3,4-d]pyrimidin-1-yl)piperidine-1-carboxylate;
ethyl 4-(4-[(3R)-3-methylmorpholin-4-yl]-6-{4-[(pyridin-4-ylcarbamoyl)amino]phenyl}-1H-pyrazolo[3,4-d]pyrimidin-1-yl)piperidine-1-carboxylate;
ethyl 4-(6-{4-[(ethylcarbamoyl)amino]phenyl}-4-[(3S)-3-methylmorpholin-4-yl]-1H-pyrazolo[3,4-d]pyrimidin-1-yl)piperidine-1-carboxylate;
ethyl 4-{6-(4-{[(2-fluoroethyl)carbamoyl]amino}phenyl)-4-[(3S)-3-methylmorpholin-4-yl]-1H-pyrazolo[3,4-d]pyrimidin-1-yl}piperidine-1-carboxylate;
ethyl 4-(4-[(3S)-3-methylmorpholin-4-yl]-6-{4-[(phenylcarbamoyl)amino]phenyl}-1H-pyrazolo[3,4-d]pyrimidin-1-yl)piperidine-1-carboxylate;
ethyl 4-(4-[(3S)-3-methylmorpholin-4-yl]-6-{4-[(pyridin-3-ylcarbamoyl)amino]phenyl}-1H-pyrazolo[3,4-d]pyrimidin-1-yl)piperidine-1-carboxylate;
ethyl 4-(4-[(3S)-3-methylmorpholin-4-yl]-6-{4-[(pyridin-4-ylcarbamoyl)amino]phenyl}-1H-pyrazolo[3,4-d]pyrimidin-1-yl)piperidine-1-carboxylate;
ethyl 4-{4-[(3S)-3-methylmorpholin-4-yl]-6-(4-{[(4-morpholin-4-ylphenyl)carbamoyl]amino}phenyl)-1H-pyrazolo[3,4-d]pyrimidin-1-yl}piperidine-1-carboxylate;
methyl 4-(6-{4-[(ethoxycarbonyl)amino]phenyl}-4-morpholin-4-yl-1H-pyrazolo[3,4-d]pyrimidin-1-yl)piperidine-1-carboxylate;
methyl 4-[6-(4-{[(2-hydroxyethoxy)carbonyl]amino}phenyl)-4-morpholin-4-yl-1H-pyrazolo[3,4-d]pyrimidin-1-yl]piperidine-1-carboxylate;
methyl 4-[6-(4-{[(2-methoxyethoxy)carbonyl]amino}phenyl)-4-morpholin-4-yl-1H-pyrazolo[3,4-d]pyrimidin-1-yl]piperidine-1-carboxylate;
methyl 4-[6-(4-{[(2-aminoethoxy)carbonyl]amino}phenyl)-4-morpholin-4-yl-1H-pyrazolo[3,4-d]pyrimidin-1-yl]piperidine-1-carboxylate;
methyl 4-{6-[4-({[2-(dimethylamino)ethoxy]carbonyl}amino)phenyl]-4-morpholin-4-yl-1H-pyrazolo[3,4-d]pyrimidin-1-yl}piperidine-1-carboxylate;
methyl 4-[4-morpholin-4-yl-6-(4-{[(2-pyrrolidin-1-ylethoxy)carbonyl]amino}phenyl)-1H-pyrazolo[3,4-d]pyrimidin-1-yl]piperidine-1-carboxylate;
methyl 4-[4-morpholin-4-yl-6-(4-{[(2-morpholin-4-ylethoxy)carbonyl]amino}phenyl)-1H-pyrazolo[3,4-d]pyrimidin-1-yl]piperidine-1-carboxylate;
methyl 4-{6-[4-({[2-(4-methylpiperazin-1-yl)ethoxy]carbonyl}amino)phenyl]-4-morpholin-4-yl-1H-pyrazolo[3,4-d]pyrimidin-1-yl}piperidine-1-carboxylate;
methyl 4-[4-morpholin-4-yl-6-(4-{[(2,2,2-trifluoroethoxy)carbonyl]amino}phenyl)-1H-pyrazolo[3,4-d]pyrimidin-1-yl]piperidine-1-carboxylate;
methyl 4-[6-(4-{[(3-hydroxypropoxy)carbonyl]amino}phenyl)-4-morpholin-4-yl-1H-pyrazolo[3,4-d]pyrimidin-1-yl]piperidine-1-carboxylate;
methyl 4-{6-[4-({[4-(4-methylpiperazin-1-yl)phenyl]carbamoyl}amino)phenyl]-4-morpholin-4-yl-1H-pyrazolo[3,4-d]pyrimidin-1-yl}piperidine-1-carboxylate;
methyl 4-[4-morpholin-4-yl-6-(4-{[(6-morpholin-4-ylpyridin-3-yl)carbamoyl]amino}phenyl)-1H-pyrazolo[3,4-d]pyrimidin-1-yl]piperidine-1-carboxylate;
methyl 4-{6-[4-({[4-(hydroxymethyl)phenyl]carbamoyl}amino)phenyl]-4-morpholin-4-yl-1H-pyrazolo[3,4-d]pyrimidin-1-yl}piperidine-1-carboxylate;
methyl 4-{6-[4-({[4-(2-hydroxyethyl)phenyl]carbamoyl}amino)phenyl]-4-morpholin-4-yl-1H-pyrazolo[3,4-d]pyrimidin-1-yl}piperidine-1-carboxylate;
methyl 4-{4-morpholin-4-yl-6-[4-({[4-(2-pyrrolidin-1-ylethyl)phenyl]carbamoyl}amino)phenyl]-1H-pyrazolo[3,4-d]pyrimidin-1-yl}piperidine-1-carboxylate;
methyl 4-{4-morpholin-4-yl-6-[4-({[4-(2-piperidin-1-ylethyl)phenyl]carbamoyl}amino)phenyl]-1H-pyrazolo[3,4-d]pyrimidin-1-yl}piperidine-1-carboxylate;
methyl 4-{4-morpholin-4-yl-6-[4-({[4-(2-piperazin-1-ylethyl)phenyl]carbamoyl}amino)phenyl]-1H-pyrazolo[3,4-d]pyrimidin-1-yl}piperidine-1-carboxylate;
methyl 4-(6-{4-[({4-[2-(4-methylpiperazin-1-yl)ethyl]phenyl}carbamoyl)amino]phenyl}-4-morpholin-4-yl-1H-pyrazolo[3,4-d]pyrimidin-1-yl)piperidine-1-carboxylate;
methyl 4-{4-morpholin-4-yl-6-[4-({[4-(2-morpholin-4-ylethyl)phenyl]carbamoyl}amino)phenyl]-1H-pyrazolo[3,4-d]pyrimidin-1-yl}piperidine-1-carboxylate;
methyl 4-{6-[4-({[4-(2-{[2-(dimethylamino)ethyl]amino}ethyl)phenyl]carbamoyl}amino)phenyl]-4-morpholin-4-yl-1H-pyrazolo[3,4-d]pyrimidin-1-yl}piperidine-1-carboxylate;
methyl 4-[6-(4-{[(4-{2-[(2-aminoethyl)amino]ethyl}phenyl)carbamoyl]amino}phenyl)-4-morpholin-4-yl-1H-pyrazolo[3,4-d]pyrimidin-1-yl]piperidine-1-carboxylate;
methyl 4-[6-(4-{[(4-{2-[(2-hydroxyethyl)amino]ethyl}phenyl)carbamoyl]amino}phenyl)-4-morpholin-4-yl-1H-pyrazolo[3,4-d]pyrimidin-1-yl]piperidine-1-carboxylate;
methyl 4-[6-(4-{[(4-{2-[(2-methoxyethyl)amino]ethyl}phenyl)carbamoyl]amino}phenyl)-4-morpholin-4-yl-1H-pyrazolo[3,4-d]pyrimidin-1-yl]piperidine-1-carboxylate;
2-hydroxyethyl(4-{4-morpholin-4-yl-1-[1-(2,2,2-trifluoroethyl)piperidin-4-yl]-1H-pyrazolo[3,4-d]pyrimidin-6-yl}phenyl)carbamate;
2-hydroxyethyl(4-{4-morpholin-4-yl-1-[1-(pyridin-3-ylmethyl)piperidin-4-yl]-1H-pyrazolo[3,4-d]pyrimidin-6-yl}phenyl)carbamate;
N-{4-[4-morpholin-4-yl-1-(tetrahydro-2H-pyran-2-yl)-1H-pyrazolo[3,4-d]pyrimidin-6-yl]phenyl}acetamide;
N-[4-(4-morpholin-4-yl-1H-pyrazolo[3,4-d]pyrimidin-6-yl)phenyl]acetamide;
1-methyl-3-[4-(4-morpholin-4-yl-1H-pyrazolo[3,4-d]pyrimidin-6-yl)phenyl]urea;
6-(1H-indol-5-yl)-4-morpholin-4-yl-1-(tetrahydro-2H-pyran-2-yl)-1H-pyrazolo[3,4-d]pyrimidine;
6-(1H-indol-5-yl)-4-morpholin-4-yl-1H-pyrazolo[3,4-d]pyrimidine;
5-[1-(1-benzylpiperidin-4-yl)-4-morpholin-4-yl-1H-pyrazolo[3,4-d]pyrimidin-6-yl]pyridin-3-ol;
1-(1-benzylpiperidin-4-yl)-6-[5-(methoxymethoxy)pyridin-3-yl]-4-morpholin-4-yl-1H-pyrazolo[3,4-d]pyrimidine;
N-(4-{4-(2-hydroxymorpholin-4-yl)-1-[1-(pyridin-3-ylmethyl)piperidin-4-yl]-1H-pyrazolo[3,4-d]pyrimidin-6-yl}phenyl)acetamide;
1-(4-{4-(2-hydroxymorpholin-4-yl)-1-[1-(pyridin-3-ylmethyl)piperidin-4-yl]-1H-pyrazolo[3,4-d]pyrimidin-6-yl}phenyl)-3-methylurea;
4-[4-morpholin-4-yl-1-(tetrahydro-2H-pyran-2-yl)-1H-pyrazolo[3,4-d]pyrimidin-6-yl]aniline;
1-methyl-3-{4-[4-morpholin-4-yl-1-(tetrahydro-2H-pyran-2-yl)-1H-pyrazolo[3,4-d]pyrimidin-6-yl]phenyl}urea;
{4-[1-(1-benzylpiperidin-4-yl)-4-morpholin-4-yl-1H-pyrazolo[3,4-d]pyrimidin-6-yl]phenyl}acetic acid;
6-[1-(1-benzylpiperidin-4-yl)-4-morpholin-4-yl-1H-pyrazolo[3,4-d]pyrimidin-6-yl]quinoline;
tert-butyl 4-[6-(4-aminophenyl)-4-morpholin-4-yl-1H-pyrazolo[3,4-d]pyrimidin-1-yl]piperidine-1-carboxylate;
1-methyl-3-{4-[4-morpholin-4-yl-1-(tetrahydro-2H-pyran-4-yl)-1H-pyrazolo[3,4-d]pyrimidin-6-yl]phenyl}urea;
1-[2-chloro-4-(4-morpholin-4-yl-1H-pyrazolo[3,4-d]pyrimidin-6-yl)phenyl]-3-methylurea;
1-(4-{4-[(2R,6S)-2,6-dimethylmorpholin-4-yl]-1-[1-(pyridin-3-ylmethyl)piperidin-4-yl]-1H-pyrazolo[3,4-d]pyrimidin-6-yl}phenyl)-3-methylurea;
3-{4-[(3R)-3-methylmorpholin-4-yl]-1-phenyl-1H-pyrazolo[3,4-d]pyrimidin-6-yl}phenol;
3-[4-(2-methylmorpholin-4-yl)-1-phenyl-1H-pyrazolo[3,4-d]pyrimidin-6-yl]phenol;
methyl 4-[6-(4-hydroxyphenyl)-4-morpholin-4-yl-1H-pyrazolo[3,4-d]pyrimidin-1-yl]piperidine-1-carboxylate;
methyl 4-(4-morpholin-4-yl-6-{4-[(phenoxycarbonyl)amino]phenyl}-1H-pyrazolo[3,4-d]pyrimidin-1-yl)piperidine-1-carboxylate;
methyl 4-(6-{4-[(methylcarbamoyl)oxy]phenyl}-4-morpholin-4-yl-1H-pyrazolo[3,4-d]pyrimidin-1-yl)piperidine-1-carboxylate;
N-{4-[-(1-isobutyrylpiperidin-4-yl)-4-morpholin-4-yl-1H-pyrazolo[3,4-d]pyrimidin-6-yl]phenyl}acrylamide;
methyl 4-[6-(4-{[(4-fluorophenoxy)carbonyl]amino}phenyl)-4-morpholin-4-yl-1H-pyrazolo[3,4-d]pyrimidin-1-yl]piperidine-1-carboxylate;
methyl 4-[6-(4-{[(Z)-(cyanoimino)(phenoxy)methyl]amino}phenyl)-4-morpholin-4-yl-1H-pyrazolo[3,4-d]pyrimidin-1-yl]piperidine-1-carboxylate;
methyl 4-[6-(4-{[(4-chlorophenoxy)carbonyl]amino}phenyl)-4-morpholin-4-yl-1H-pyrazolo[3,4-d]pyrimidin-1-yl]piperidine-1-carboxylate;
tert-butyl 4-(6-{4-[(methylsulfamoyl)amino]phenyl}-4-morpholin-4-yl-1H-pyrazolo[3,4-d]pyrimidin-1-yl)piperidine-1-carboxylate;
tert-butyl 4-[6-(4-{[(6-fluoropyridin-3-yl)carbamoyl]amino}phenyl)-4-morpholin-4-yl-1H-pyrazolo[3,4-d]pyrimidin-1-yl]piperidine-1-carboxylate;
tert-butyl 4-{6-[4-({[4-(hydroxymethyl)phenyl]carbamoyl}amino)phenyl]-4-morpholin-4-yl-1H-pyrazolo[3,4-d]pyrimidin-1-yl}piperidine-1-carboxylate;
tert-butyl 4-{6-[4-({[4-(2-hydroxyethyl)phenyl]carbamoyl}amino)phenyl]-4-morpholin-4-yl-1H-pyrazolo[3,4-d]pyrimidin-1-yl}piperidine-1-carboxylate;
1-(4-{3-[3-(dimethylamino)prop-1-yn-1-yl]-1-ethyl-4-morpholin-4-yl-1Hpyrazolo[3,4-d]pyrimidin-6-yl}phenyl)-3-methylurea;
1-{4-[1-ethyl-3-(3-hydroxyprop-1-yn-1-yl)-4-morpholin-4-yl-1H-pyrazolo[3,4-d]pyrimidin-6-yl]phenyl}-3-pyridin-3-ylurea;
4-{4-[6-(1H-indol-5-yl)-4-morpholin-4-yl-1H-pyrazolo[3,4-d]pyrimidin-1-yl]piperidin-1-yl}-N,N-dimethyl-4-oxobut-2-en-1-amine;
N2,N2-dimethyl-N-[4-(4-morpholin-4-yl-1H-pyrazolo[3,4-d]pyrimidin-6-yl)phenyl]glycinamide;
methyl(4-{1-[1-(4-fluorobenzyl)piperidin-4-yl]-4-morpholin-4-yl-1Hpyrazolo[3,4-d]pyrimidin-6-yl}phenyl)carbamate;
N-(4-{4-(2-hydroxymorpholin-4-yl)-1-[1-(pyridin-3-ylmethyl)piperidin-4-yl]-1Hpyrazolo[3,4-d]pyrimidin-6-yl}phenyl)acetamide;
1-(4-{4-(2-hydroxymorpholin-4-yl)-1-[1-(pyridin-3-ylmethyl)piperidin-4-yl]-1Hpyrazolo[3,4-d]pyrimidin-6-yl}phenyl)-3-methylurea;
3-[4-(1,4-oxazepan-4-yl)-1-phenyl-1H-pyrazolo[3,4-d]pyrimidin-6-yl]phenol;
3-(1-phenyl-4-thiomorpholin-4-yl-1H-pyrazolo[3,4-d]pyrimidin-6-yl)phenol;
and 3-(3-fluoro-4-morpholin-4-yl-1-phenyl-1H-pyrazolo[3,4-d]pyrimidin-6-yl)phenol.

53. A compound selected from the group consisting of 3-[4-(1,4-oxazepan-4-yl)-1-phenyl-1H-pyrazolo[3,4-d]pyrimidin-6-yl]phenol and 3-(1-phenyl-4-thiomorpholin-4-yl-1H-pyrazolo[3,4-d]pyrimidin-6-yl)phenol.

54. A composition comprising a compound of claim 1 and a pharmaceutically acceptable carrier.

55. The composition of claim 64, wherein the pharmaceutically acceptable carrier is suitable for oral administration and the composition comprises an oral dosage form.

56. A composition comprising a compound of claim 17 and a pharmaceutically acceptable carrier.

57. The composition of claim 66, wherein the pharmaceutically acceptable carrier is suitable for oral administration and the composition comprises an oral dosage form.

58. A composition comprising a compound of claim 37 and a pharmaceutically acceptable carrier.

59. The composition of claim 68, wherein the pharmaceutically acceptable carrier is suitable for oral administration and the composition comprises an oral dosage form.

60. A composition comprising a compound of claim 1; a second compound selected from the group consisting of a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, and lavendustin A; and a pharmaceutically acceptable carrier.

61. The composition of claim 70, wherein the second compound is Avastin.

62. A composition comprising a compound of claim 17; a second compound selected from the group consisting of a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, and lavendustin A; and a pharmaceutically acceptable carrier.

63. The composition of claim 62, wherein the second compound is Avasfin.

64. A composition comprising a compound of claim 27; a second compound selected from the group consisting of a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, and lavendustin A; and a pharmaceutically acceptable carrier.

65. The composition of claim 64, wherein the second compound is Avasfin.

66. A method of treating a mTOR-related disorder, comprising administering to a mammal in need thereof a compound of claim 1 in an amount effective to treat a mTOR-related disorder.

67. The method of claim 66, wherein the mTOR-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

68. The method of claim 67, wherein the mTOR-related disorder is cancer.

69. The method of claim 68, wherein the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

70. A method of treating a mTOR-related disorder, comprising administering to a mammal in need thereof a compound of claim 17 in an amount effective to treat a mTOR-related disorder.

71. The method of claim 70, wherein the mTOR-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

72. The method of claim 71, wherein the mTOR-related disorder is cancer.

73. The method of claim 72, wherein the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

74. A method of treating a mTOR-related disorder, comprising administering to a mammal in need thereof a compound of claim 27 in an amount effective to treat a mTOR-related disorder.

75. The method of claim 74, wherein the mTOR-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

76. The method of claim 75, wherein the mTOR-related disorder is cancer.

77. The method of claim 76, wherein the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

78. A method of treating a PI3K-related disorder, comprising administering to a mammal in need thereof a compound of claim 1 in an amount effective to treat a PI3K-related disorder.

79. The method of claim 78, wherein the PI3K-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

80. The method of claim 79, wherein the PI3K-related disorder is cancer.

81. The method of claim 80, wherein the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

82. A method of treating a PI3K-related disorder, comprising administering to a mammal in need thereof a compound of claim 17 in an amount effective to treat a PI3K-related disorder.

83. The method of claim 82, wherein the PI3K-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

84. The method of claim 83, wherein the PI3K-related disorder is cancer.

85. The method of claim 84, wherein the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

86. A method of treating a PI3K-related disorder, comprising administering to a mammal in need thereof a compound of claim 27 in an amount effective to treat a PI3K-related disorder.

87. The method of claim 86, wherein the PI3K-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

88. The method of claim 87, wherein the PI3K-related disorder is cancer.

89. The method of claim 88, wherein the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

90. A method of treating a cancer selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer comprising administering to a mammal in need thereof the composition of claim 60 in an amount effective to treat the cancer.

91. A method of treating a cancer selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer comprising administering to a mammal in need thereof the composition of claim 62 in an amount effective to treat the cancer.

92. A method of treating a cancer selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer comprising administering to a mammal in need thereof the composition of claim 64 in an amount effective to treat the cancer.

93. A method of inhibiting mTOR in a subject, comprising administering to a subject in need thereof a compound of claims 1 in an amount effective to inhibit mTOR.

94. A method of inhibiting PI3K in a subject, comprising administering to a subject in need thereof a compound of claim 1 in an amount effective to inhibit PI3K.

95. A method of inhibiting mTOR in a subject, comprising administering to a subject in need thereof a compound of claim 17 in an amount effective to inhibit mTOR.

96. A method of inhibiting PI3K in a subject, comprising administering to a subject in need thereof a compound of claim 17 in an amount effective to inhibit PI3K.

97. A method of inhibiting mTOR in a subject, comprising administering to a subject in need thereof a compound of claim 27 in an amount effective to inhibit mTOR.

98. A method of inhibiting PI3K in a subject, comprising administering to a subject in need thereof a compound of claim 27 in an amount effective to inhibit PI3K.

99. A method of synthesizing a compound of claim 1 comprising:
a) reacting a compound of the formula (IV): 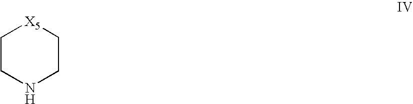
wherein X₅ is —O—, —CH₂—O—, or —S(O)ₙ— wherein any one or more of the ring carbons of the compound of formula (IV) can independently be substituted with C₁-C₃alkyl, C₂-C₆alkenyl, C₂-C₆alkynyl, C₁-C₃alkoxy, C₁-C₃acyl, C₁-C₃alkoxycarbonyl, amino(C₁-C₆alkyl), hydroxyl, fluorine, or —CN, where any two hydrogen atoms attached to the same carbon atom can be replaced by an oxygen atom, the oxygen atom taken together with the carbon to which it is attached, forming a carbonyl (C═O),
and wherein n is an integer from 0 to 2;
with a compound of the formula V: 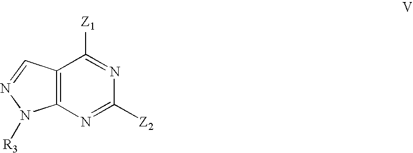
wherein Z₁ and Z₂ are each independently a halogen;
R₃ is as defined in claim 1;
thereby providing a compound having the formula VI: 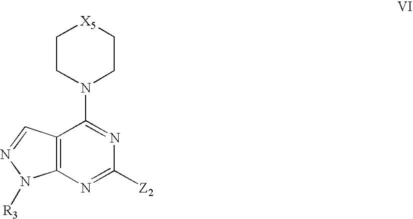
wherein any one or more of the ring hydrogen atoms of the group 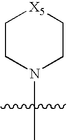
of Formula VI can independently be replaced with C₁-C₆alkyl, C₂-C₆alkenyl, C₂-C₆alkynyl, C₁-C₃alkoxy, C₁-C₃acyl, C₁-C₃alkoxycarbonyl, amino(C₁-C₆alkyl), hydroxyl, fluorine, or —CN, where any two hydrogen atoms attached to the same carbon atom can be replaced by an oxygen atom, the oxygen atom taken together with the carbon to which it is attached, forming a carbonyl (C═O),
b) reacting the compound of formula VI with a boronic acid of the structure:
R₂B(OH)₂
wherein R₂ is as defined in claim 1, thereby providing a compound of the formula VII: 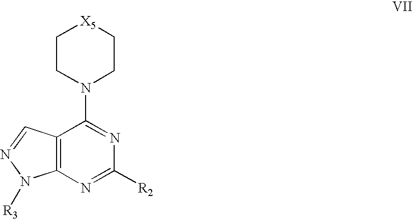
wherein any one or more of the ring hydrogen atoms of the group 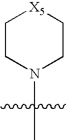
in Formula VII can independently be replaced with C₁-C₆alkyl, C₂-C₆alkenyl, C₂-C₆alkynyl, C₁-C₃alkoxy, C₁-C₃acyl, C₁-C₃alkoxycarbonyl, amino(C₁-C₆alkyl), hydroxyl, fluorine, or —CN, where any two hydrogen atoms attached to the same carbon atom can be replaced by an oxygen atom, the oxygen atom taken together with the carbon to which it is attached, forming a carbonyl (C═O).
